(19) United States
(12) Patent Application Publication
(10) Pub. No.: US 2026/0297105 A1
(43) Pub. Date: Oct. 1, 2026

(54) RAF-MEK PROTEIN COMPLEX MODULATORS AND METHODS OF USE THEREOF

(71) Applicant: GANDEEVA THERAPEUTICS, INC., Seattle, WA (US)

(72) Inventors: Jason B. CRAWFORD, Seattle, WA (US); Ravi Shashi Nayana MUNUGANTI, Seattle, WA (US); Mahesh THAKKAR, Seattle, WA (US); Athisayamani Jeyaraj DURAISWAMY, Seattle, WA (US); Jegadeesan SAKTHIVEL, Seattle, WA (US)

(73) Assignee: GANDEEVA THERAPEUTICS, INC., Seattle, WA (US)

(21) Appl. No.: 19/476,685

(22) PCT Filed: Apr. 18, 2024
(86) PCT No.: PCT/US2024/025259

§ 371 Date: Oct. 18, 2025

Related U.S. Application Data
(60) Provisional application No. 63/497,149, filed on Apr. 19, 2023.

(51) Int. Cl. C07D 491/107; A61K 31/536; A61K 31/5365; A61K 31/537; A61K 31/5377; C07B 59/00; C07D 265/18; C07D 413/04; C07D 413/06; C07D 413/10; C07D 413/12; C07D 413/14; C07D 498/04; C07D 498/10

(52) U.S. Cl. CPC C07D 491/107; A61K 31/536; A61K 31/5365; A61K 31/537; A61K 31/5377; C07B 59/002; C07D 265/18; C07D 413/04; C07D 413/06; C07D 413/10; C07D 413/12; C07D 413/14; C07D 498/04; C07D 498/10; C07B 2200/05

(57) ABSTRACT

Described herein are compounds and compositions useful for preventing and/or treating a disease or condition, e.g., a disease or condition associated with MAPK signaling pathway, e.g., cancer. In one aspect, the present disclosure provides a compound of formula (I): (I), or a pharmaceutically acceptable salt thereof, wherein the variables are as defined herein.

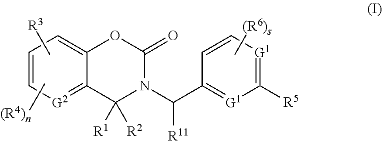

### CROSS-REFERENCE TO RELATED APPLICATIONS

This application claims priority to and the benefit of U.S. Provisional Patent Application No. 63/497,149, filed on Apr. 19, 2023, the entire contents of which are incorporated herein by reference.

### FIELD OF THE INVENTION

The invention relates generally to compounds and compositions useful for preventing and/or treating a disease, disorder or condition associated with a MAPK signaling pathway, e.g., cancer.

### BACKGROUND

The mitogen-activated protein kinase (MAPK) pathway mediates the activity of a number of effector molecules (e.g., other cell cycle regulatory proteins) that coordinate the control of cellular proliferation, survival, differentiation, and migration (McCubrey J. A. et al. (2007) BIOCHEM. BIOPHYS. ACTA Aug; 1773(8)). To activate the pathway, a cell signal, such as a growth factor, cytokine, or hormone, binds to a cell surface receptor, such as Rat sarcoma virus protein (RAS). Once bound, the RAS then binds a Guanosine-5′-triphosphate (GTP) molecule and activates rapidly accelerated fibrosarcoma protein (RAF). Once activated, RAF can directly phosphorylate mitogen/extracellular-signal-regulated kinase kinase (MEK, also known as Map Kinase Kinase (MKK)), and then MEK can phosphorylate the extracellular signal-related kinase (ERK). Activated ERK then phosphorylates a wide array of effector molecules, e.g., kinases, phosphatases, transcription factors, and cytoskeletal proteins, thereby effecting cell cycle changes (Bafico A. et al. (2003) Signaling Pathways of Tyrosine Kinase Receptors, Holland-Frei CANCER MEDICINE 6th edition. Hamilton (ON): BC Decker). As a result, the RAS-RAF-MEK-ERK signaling pathway can transmit signals from cell surface receptors to the nucleus, and is essential, e.g., in cell proliferation and survival (Barbosa R. et al. (2021) MOL. CANCER RES. 19(3):361-374. doi: 10.1158/1541-7786.MCR-20-0687). This signaling cascade is further regulated by the multiple isoforms of RAS (including KRAF, NRAS and HRAS), RAF (ARAF, BRAF, CRAF/RAF-1), MEK (MEK-1 and MEK-2) and ERK (ERK-1 and ERK-2).

Research continues to show this pathway regulates key cellular activities including proliferation, differentiation, survival, and angiogenesis (Zheng B. et al. (2003) BLOOD 102 (3): 1019-1027). Inappropriate activation of proteins in this pathway can lead to increased cell proliferation, a key characteristic of many cancers, such as melanoma, non-small cell lung, colorectal, and thyroid cancers. The MAPK/ERK signaling pathway plays an important role in tumor invasion and metastasis (McCubrey J. A. et al. (2007) supra).

Approximately 30% of all human cancers have been found to have a constitutively activated MAPK/ERK pathway, the most common mechanisms being a mutated KRAS or BRAF, and a constitutively activated MEKI, which results in uncontrolled cell growth. Despite the advances made to date in the treatment of cancers and other diseases and conditions mediated by MAPK/ERK pathway, there is an ongoing need for additional therapies for treating such cancers, diseases and conditions.

### SUMMARY

Described herein are compounds and compositions useful for preventing and/or treating a disease or condition associated with MAPK signaling pathway, e.g., cancer. The compounds described herein may act as a molecular glue and modulate the interaction of certain protein complexes within the MAPK signaling pathway. For example, the compounds disclosed herein may bind to the interface region between a RAF (rapidly accelerated fibrosarcoma) protein, e.g., BRAF protein, and a MEK (mitogen-activated protein kinase kinase) protein and modulate the activity and down signaling mediated by the RAF-MEK protein complex, e.g., a BRAF-MEK protein complex.

In one aspect, the present disclosure features a compound of Formula (I):

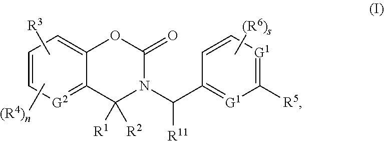

or a pharmaceutically acceptable salt thereof, wherein the variables are as defined herein.

In some embodiments, the compound is a compound of formula (I-1):

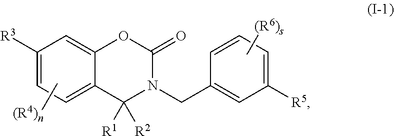

or a pharmaceutically acceptable salt thereof, wherein the variables are as defined herein.

In certain embodiments, the compound is a compound of formula (IA):

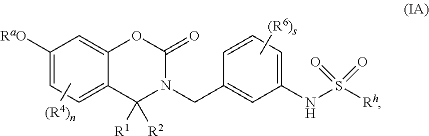

or a pharmaceutically acceptable salt thereof, wherein the variables are as defined herein.

In other embodiments, the compound is a compound of formula (IA-1):

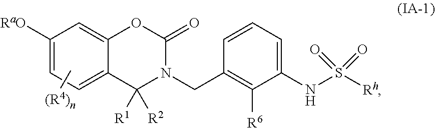

or a pharmaceutically acceptable salt thereof, wherein the variables are as defined herein.

In some embodiments, the compound is a compound of formula (IB):

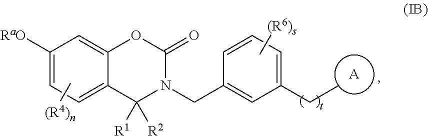

or a pharmaceutically acceptable salt thereof, wherein the variables are as defined herein.

In certain embodiments, the compound is a compound of formula (IB-1):

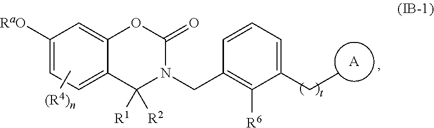

or a pharmaceutically acceptable salt thereof, wherein the variables are as defined herein.

In some embodiments, the compound is a compound of Formula (IC):

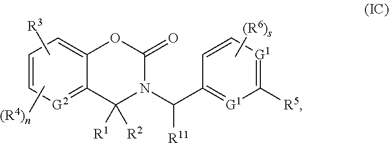

or a pharmaceutically acceptable salt thereof, wherein:
R¹ and R² are taken together with the carbon atom to which they are attached to form C₃₋₆ cycloalkyl or 3-6 membered heterocyclyl ring, wherein the C₃₋₆ cycloalkyl or 3-6 membered heterocyclyl ring may be optionally substituted; and
the remaining variables are as defined herein.

In some embodiments, the compound is a compound of Formula (IC-1):

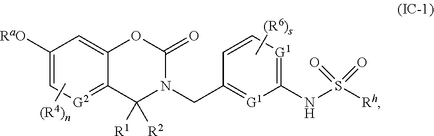

or a pharmaceutically acceptable salt thereof, wherein:
R¹ and R² are taken together with the carbon atom to which they are attached to form C₃₋₆ cycloalkyl or 3-6 membered heterocyclyl ring, wherein the C₃₋₆ cycloalkyl or 3-6 membered heterocyclyl ring may be optionally substituted; and
the remaining variables are as defined herein.

In some embodiments, the compound is a compound of Formula (IC-2):

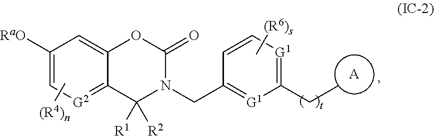

or a pharmaceutically acceptable salt thereof, wherein:
R¹ and R² are taken together with the carbon atom to which they are attached to form C₃₋₆ cycloalkyl or 3-6 membered heterocyclyl ring, wherein the C₃₋₆ cycloalkyl or 3-6 membered heterocyclyl ring may be optionally substituted; and
the remaining variables are as defined herein.

In certain embodiments, the compound is a compound of Formula (ID):

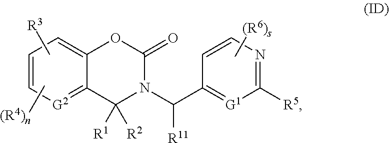

or a pharmaceutically acceptable salt thereof, wherein the variables are as defined herein.

In some embodiments, the compound is a compound of Formula (IE):

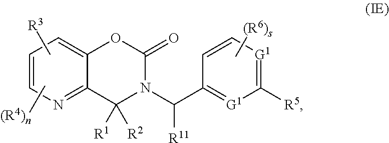

or a pharmaceutically acceptable salt thereof, wherein the variables are as defined herein.

In certain embodiments, the compound is a compound of Formula (IF):

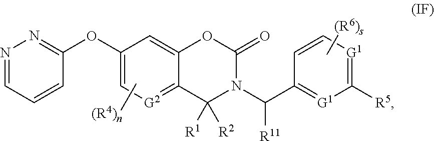

or a pharmaceutically acceptable salt thereof, wherein the variables are as defined herein.

In some embodiments, the compound is a compound of Formula (IF-1):

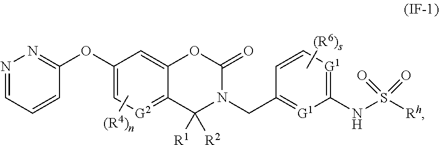

or a pharmaceutically acceptable salt thereof, wherein the variables are as defined herein.

In some embodiments, the compound is a compound of Formula (IF-2):

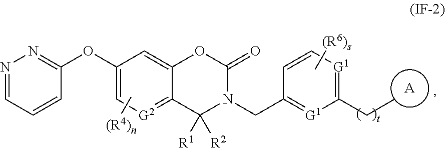

or a pharmaceutically acceptable salt thereof, wherein the variables are as defined herein.

In some embodiments, the compound is a compound of Formula (IG):

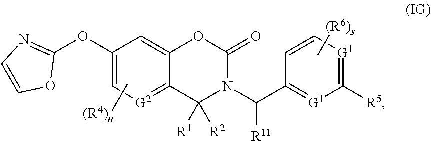

or a pharmaceutically acceptable salt thereof, wherein the variables are as defined herein.

In some embodiments, the compound is a compound of Formula (IG-1):

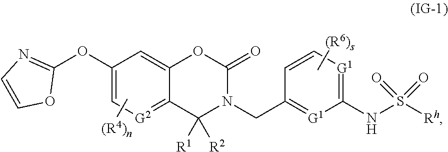

or a pharmaceutically acceptable salt thereof, wherein the variables are as defined herein.

In some embodiments, the compound is a compound of Formula (IG-2):

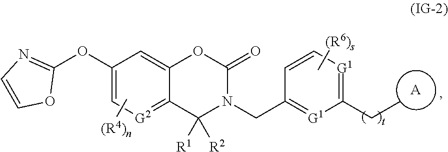

or a pharmaceutically acceptable salt thereof, wherein the variables are as defined herein.

Also provided herein is a pharmaceutical composition comprising a compound described herein or a pharmaceutically acceptable salt thereof, and a pharmaceutically acceptable excipient.

In another aspect, the disclosure provides a method of modulating the interaction between RAF and MEK in a RAF-MEK complex, e.g., BRAF and MEK in a BRAF-MEK complex. The method comprises contacting BRAF, MEK or a combination of BRAF and MEK with an effective amount of a compound disclosed therein or a pharmaceutically acceptable salt thereof, or a pharmaceutical composition disclosed herein thereby to modulate the interaction between RAF (e.g., BRAF) and MEK. The modulation can involve stabilizing or destabilizing the interaction between RAF and MEK in a RAF-MEK complex, e.g., BRAF and MEK in a BRAF-MEK complex.

In another aspect, the present disclosure provides a method of treating a disease or condition associated with MAPK signaling pathway (e.g., cancer) in a subject in need thereof. The method comprises administering to the subject a therapeutically effective amount of a compound disclosed herein or a pharmaceutically acceptable salt thereof, or a pharmaceutical composition disclosed herein.

Other objects and advantages will become apparent to those skilled in the art from a consideration of the ensuing Detailed Description, Examples, and Claims.

### DETAILED DESCRIPTION

As generally described herein, the present disclosure provides compounds and compositions useful for preventing and/or treating a disease or condition described herein, e.g., a disease, disorder, or condition associated with MAPK signaling pathway, e.g., cancer.

### I. Definitions

### Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{d} Edition, Cambridge University Press, Cambridge, 1987.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, IN 1972). The invention additionally encompasses compounds described herein as individual isomers substantially free of other isomers, and alternatively, as mixtures of various isomers.

As used herein a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises more than 75% by weight, more than 80% by weight, more than 85% by weight, more than 90% by weight, more than 91% by weight, more than 92% by weight, more than 93% by weight, more than 94% by weight, more than 95% by weight, more than 96% by weight, more than 97% by weight, more than 98% by weight, more than 98.5% by weight, more than 99% by weight, more than 99.2% by weight, more than 99.5% by weight, more than 99.6% by weight, more than 99.7% by weight, more than 99.8% by weight or more than 99.9% by weight, of the enantiomer. In certain embodiments, the weights are based upon total weight of all enantiomers or stereoisomers of the compound.

In the compositions provided herein, an enantiomerically pure compound can be present with other active or inactive ingredients. For example, a pharmaceutical composition comprising enantiomerically pure R-compound can comprise, for example, about 90% excipient and about 10% enantiomerically pure R-compound. In certain embodiments, the enantiomerically pure R-compound in such compositions can, for example, comprise, at least about 95% by weight R-compound and at most about 5% by weight S-compound, by total weight of the compound. For example, a pharmaceutical composition comprising enantiomerically pure S-compound can comprise, for example, about 90% excipient and about 10% enantiomerically pure S-compound. In certain embodiments, the enantiomerically pure S-compound in such compositions can, for example, comprise, at least about 95% by weight S-compound and at most about 5% by weight R-compound, by total weight of the compound. In certain embodiments, the active ingredient can be formulated with little or no excipient or carrier.

Compound described herein may also comprise one or more isotopic substitutions. For example, H may be in any isotopic form, including ¹H, ²H (D or deuterium), and ³H (T or tritium); C may be in any isotopic form, including ¹²C, ¹³C₃₋₆, and ¹⁴C; O may be in any isotopic form, including ¹⁶O and 180; F may be in any isotopic form, including ¹⁸F and ¹⁹F; and the like.

The following terms are intended to have the meanings presented therewith below and are useful in understanding the description and intended scope of the present invention. When describing the invention, which may include compounds and pharmaceutically acceptable salts thereof, pharmaceutical compositions containing such compounds and methods of using such compounds and compositions, the following terms, if present, have the following meanings unless otherwise indicated. It should also be understood that when described herein any of the moieties defined forth below may be substituted with a variety of substituents, and that the respective definitions are intended to include such substituted moieties within their scope as set out below. Unless otherwise stated, the term “substituted” is to be defined as set out below. It should be further understood that the terms “groups” and “radicals” can be considered interchangeable when used herein. The articles “a” and “an” may be used herein to refer to one or to more than one (i.e. at least one) of the grammatical objects of the article. By way of example “an analogue” means one analogue or more than one analogue.

When a range of values is listed, it is intended to encompass each value and sub-range within the range. For example, “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl.

As used herein, “alkyl” refers to a radical of a straight-chain or branched saturated hydrocarbon group, e.g., having 1 to 20 carbon atoms (“C₁₋₂₀ alkyl”). In some embodiments, an alkyl group has 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). Examples of C₁₋₆ alkyl groups include methyl, ethyl, propyl, isopropyl, butyl, isobutyl, pentyl, hexyl, and the like.

As used herein, “alkenyl” refers to a radical of a straight-chain or branched hydrocarbon group having from 2 to 20 carbon atoms, one or more carbon-carbon double bonds (e.g., 1, 2, 3, or 4 carbon-carbon double bonds), and optionally one or more carbon-carbon triple bonds (e.g., 1, 2, 3, or 4 carbon-carbon triple bonds) (“C₂₋₂₀ alkenyl”).

As used herein, “alkynyl” refers to a radical of a straight-chain or branched hydrocarbon group having from 2 to 20 carbon atoms, one or more carbon-carbon triple bonds (e.g., 1, 2, 3, or 4 carbon-carbon triple bonds), and optionally one or more carbon-carbon double bonds (e.g., 1, 2, 3, or 4 carbon-carbon double bonds) (“C_{2_20} alkynyl”).

As used herein, “alkylene,” refers to a divalent radical of an alkyl. When a range or number of carbons is provided for a particular “alkylene” group, it is understood that the range or number refers to the range or number of carbons in the linear carbon divalent chain. “Alkylene” group may be substituted or unsubstituted with one or more substituents as described herein.

As used herein, “aryl” refers to a radical of a monocyclic or polycyclic (e.g., bicyclic or tricyclic) 4n+2 aromatic ring system (e.g., having 6, 10, or 14π electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₋₄ aryl”; e.g., anthracyl).

As used herein, “heteroaryl” refers to a radical of a 5-10 membered monocyclic or bicyclic 4n+2 aromatic ring system (e.g., having 6 or 10 electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur.

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl.

Examples of representative heteroaryls include the following:

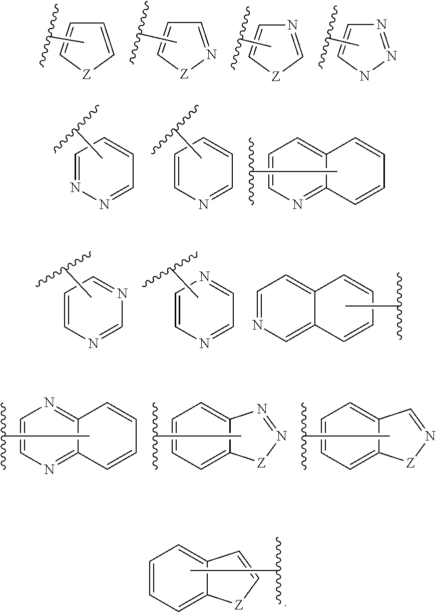

wherein each Z is selected from carbonyl, N, NR⁶⁵, O, and S; and R⁶⁵ is independently hydrogen, C₁-C₅ alkyl, C₃-C₁₀ carbocyclyl, 4-10 membered heterocyclyl, C₆-C₁₀ aryl, and 5-10 membered heteroaryl.

As used herein, “carbocyclyl” or “carbocyclic” refers to a radical of a non-aromatic cyclic hydrocarbon group having from 3 to 10 ring carbon atoms (“C₃₋₁₀ carbocyclyl”) and zero heteroatoms in the non-aromatic ring system. In some embodiments, a carbocyclyl group has 3 to 8 ring carbon atoms (“C₃₋₈ carbocyclyl”).

The term “cycloalkyl” refers to a monovalent saturated cyclic, bicyclic, or bridged cyclic (e.g., adamantyl) hydrocarbon group of 3-12, 3-8, 4-8, or 4-6 carbons, referred to herein, e.g., as “C₄₋₈cycloalkyl,” derived from a cycloalkane. Exemplary cycloalkyl groups include, but are not limited to, cyclohexanes, cyclopentanes, cyclobutanes and cyclopropanes. Unless specified otherwise, cycloalkyl groups are optionally substituted at one or more ring positions with, for example, alkanoyl, alkoxy, alkyl, haloalkyl, alkenyl, alkynyl, amido, amidino, amino, aryl, arylalkyl, azido, carbamate, carbonate, carboxy, cyano, cycloalkyl, ester, ether, formyl, halogen, haloalkyl, heteroaryl, heterocyclyl, hydroxyl, imino, ketone, nitro, phosphate, phosphonato, phosphinato, sulfate, sulfide, sulfonamido, sulfonyl or thiocarbonyl. Cycloalkyl groups can be fused to other cycloalkyl, aryl, or heterocyclyl groups. In certain embodiments, the cycloalkyl group is not substituted, i.e., it is unsubstituted.

As used herein, “heterocyclyl” or “heterocyclic” refers to a radical of a 3- to 10-membered non-aromatic ring system having ring carbon atoms and 1 to 4 ring heteroatoms, wherein each heteroatom is independently selected from nitrogen, oxygen, sulfur, boron, phosphorus, and silicon (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more carbocyclyl or cycloalkyl groups wherein the point of attachment is either on the carbocyclyl, cycloalkyl, or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system.

In some embodiments, a heterocyclyl group is a 5-10 membered non-aromatic ring system having ring carbon atoms and 1-4 ring heteroatoms, wherein each heteroatom is independently selected from nitrogen, oxygen, sulfur, boron, phosphorus, and silicon (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has one ring heteroatom selected from nitrogen, oxygen, and sulfur.

Exemplary 3-membered heterocyclyl groups containing one heteroatom include, without limitation, azirdinyl, oxiranyl, thiorenyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

“Hetero” when used to describe a compound or a group present on a compound means that one or more carbon atoms in the compound or group have been replaced by a nitrogen, oxygen, or sulfur heteroatom. Hetero may be applied to any of the hydrocarbyl groups described above such as alkyl, e.g., heteroalkyl; carbocyclyl, e.g., heterocyclyl; aryl, e.g,. heteroaryl; and the like having from 1 to 5, and particularly from 1 to 3 heteroatoms.

As used herein, “halo” or “halogen” refers to fluoro (F), chloro (Cl), bromo (Br) and iodo (I). In certain embodiments, the halo group is either fluoro or chloro.

As used herein, “haloalkyl” refers to an alkyl group substituted with one or more halogen atoms.

As used herein, “oxo” refers to —C═O.

In general, the term “substituted,” whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position.

Unless otherwise indicated, an “optionally substituted” group may have a suitable substituent at each substitutable position of the group, and when more than one position in any given structure may be substituted with more than one substituent selected from a specified group, the substituent may be either the same or different at each position. Combinations of substituents envisioned under this invention are preferably those that result in the formation of stable or chemically feasible compounds.

Suitable monovalent substituents on a substitutable carbon atom of an “optionally substituted” group (such as an alkyl, alkenyl, alkynyl, alkylene, alkenylene, alkynylene or the carbon atom of a carbocyclyl, aryl, heterocyclyl or heteroaryl) are independently, for example, halogen; —(CH₂)₀₋₄R^{o}; —(CH₂)₀₋₄OR^{o}; —O—(CH₂)₀₋₄C(O)OR^{o}; —(CH₂)₀₋₄CH(OR^{o})₂; —(CH₂)₀₋₄SR^{o}; —(CH₂)₀₋₄Ph, which may be substituted with R^{o}; —(CH₂)₀₋₄O(CH₂)₀₋₁Ph, which may be substituted with R^{o}; —CH═CHPh, which may be substituted with —NO₂; —CN; —N₃; —(CH₂)₀₋₄N(R^{o})₂; —(CH₂)₀₋₄N(R^{o})C(O)R^{o}; —N(R^{o})C(S)R^{o}; —(CH₂)₀₋₄N(R^{o}) C(O)NR^{o}₂; —N(R^{o})C(S)NR^{o}2; —(CH₂)₀₋₄N(R^{o})C(O)OR^{o}; —N(R^{o})N(R^{o})C(O)R^{o}; —N(R^{o})N(R^{o})C(O)NR^{o}₂; —N(R^{o})N(R^{o})C(O)OR^{o}; —(CH₂)O₄C(O)R^{o}; —C(S)R^{o}; —(CH₂)₀₋₄C(O)OR^{o}; —(CH₂)₀₋₄C(O)SR^{o}; —(CH₂)₀₋₄C(O)OSiR^{o}₃; —(CH₂)₀₋₄OC(O)R^{o}; —OC(O)(CH₂)₀₋₄SR^{o}—, SC(S)SR^{o}; —(CH₂)₀₋₄SC(O)R^{o}; —(CH₂)₀₋₄C(O)NR^{o}₂; —C(S)NR^{o}₂; —C(S)SR^{o}; —(CH₂)₀₋₄OC(O)NR^{o}₂; —C(O)N(OR^{o})R^{o}; —C(O)C(O)R^{o}; —C(O)CH₂C(O)R^{o}; —C(NOR^{o})R^{o}; —(CH₂)₀₋₄SSR^{o}; —(CH₂)O₄S(O)₂R^{o}; —(CH₂)₀₋₄S(O)₂OR^{o}; —(CH₂)₀₋₄₀S(O)₂R^{o}; —S(O)₂NR^{o}₂; —(CH₂)₀₋₄S(O)R^{o}; —N(R^{o})S(O)₂NR^{o}₂; —N(R^{o})S(O)₂R^{o}; —N(OR^{o})R^{o}; —C(NH)NR^{o}2; —P(O)₂R^{o}; —P(O)R^{o}₂; —OP(O)R^{o}2; —OP(O)(OR^{o})₂; —SiR₃; —(C₁₋₄ straight or branched alkylene)O—N(R^{o})₂; or —(C₁₋₄ straight or branched alkylene)C(O)O—N(R^{o})₂, where each R^{o} may be substituted as defined below and is independently hydrogen, C₁₋₆ alkyl, C₁₋₆ alkenyl, C₁₋₆ alkynyl, —CH₂Ph, —O(CH₂)₀₋₁Ph, or a 5-6-membered saturated, partially unsaturated, or aryl ring having 0-4 heteroatoms independently selected from nitrogen, oxygen, or sulfur, or, notwithstanding the definition above, two independent occurrences of R^{o}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{o} (or the ring formed by taking two independent occurrences of R^{o} together with their intervening atoms), are independently, for example, deuterium, halogen, —(CH₂)₀₋₂R^{•}, -(haloR^{•}), —(CH₂)₀₋₂OH, —(CH₂)₀₋₂OR^{•}, —(CH₂)₀₋₂CH(OR^{•})₂; —O(haloR^{•}), —CN, —N₃, —(CH₂)₀₋₂C(O)R^{•}, —(CH₂)₀₋₂C(O)OH, —(CH₂)₀₋₂C(O)OR^{•}, —(CH₂)₀₋₂SR^{•}, —(CH₂)₀₋₂SH, —(CH₂)₀₋₂NH₂, —(CH₂)₀₋₂NHR^{•}, —(CH₂)₀₋₂NR^{•}₂, —NO₂, —SiR^{•}3, —OSiR^{•}3, —C(O)SR^{•}, —(C₁₋₄ straight or branched alkylene)C(O)OR^{•}, or —SSR^{•}, where each R^{•} is unsubstituted or where preceded by “halo” is substituted only with one or more halogens, and is independently selected from C₁₋₄ alkyl, C₁₋₄ alkenyl, C₁₋₄ alkynyl, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{o} include ═O and =S.

Suitable divalent substituents on a saturated carbon atom of an “optionally substituted” group include the following: ═O, ═S, ═NNR^{•}₂, ═NNHC(O)R^{•}, ═NNHC(O)OR^{•}, ═NNHS(O)₂R^{•}, ═NR^{•}, ═NOR^{•}, —O(C(R^{•}₂))₂₋₃O—, or —S(C(R^{•}₂))₂₋₃S—, where each independent occurrence of R^{•} is selected from hydrogen, C₁₋₆ alkyl, C₁₋₆ alkenyl, C₁₋₆ alkynyl, and carbocyclyl, which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(C(R^{•})₂)₂₋₃O—, where each independent occurrence of R^{•} is selected from hydrogen, C₁₋₆ alkyl, C₁₋₆ alkenyl, C₁₋₆ alkynyl, and carbocyclyl, which may be substituted as defined below, or an unsubstituted 5-6-membered saturated, partially unsaturated, or aryl ring having 0-4 heteroatoms independently selected from nitrogen, oxygen, or sulfur.

Suitable substituents on the alkyl, alkenyl, alkynyl, or carbocyclyl group of R^{•} include halogen, —R^{•}, -(haloR^{•}), —OH, —OR^{•}, —O(haloR^{•}), —CN, —C(O)OH, —C(O)OR^{•}, —NH2, —NHR^{•}, —NR^{•}₂, or —NO2, where each R^{•} is unsubstituted or where preceded by “halo” is substituted only with one or more halogens, and is independently C₁₋₆ alkyl, C₁-s alkenyl, C₁₋₆ alkynyl, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; where each R^{†} is independently hydrogen, C₁₋₆ alkyl, C₁₋₆ alkenyl, C₁₋₆ alkynyl, which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur. In certain embodiments, it should be understood that a substitutable nitrogen can be substituted with four substituents (or have four bonds to less than four substituents) such that the nitrogen atom will be positively charged or its cation, i.e., N⁺. Such substitution of a substitutable nitrogen can be of a ring nitrogen, for example, a ring nitrogen of a heterocyclic group containing nitrogen as a ring atom. In particular embodiments, where one of the substituents of a cationic nitrogen atom is a hydroxyl group, the hydroxyl group can be deprotonated and represented by a negatively charged oxygen atom, i.e., O⁻. In such cases, a general formula for the substitution can be represented by RN⁺(O⁻)(R′)R″, where R, R′ and R″ represent carbon or other atoms, groups and/or moieties to which the nitrogen atom is bound.

Suitable substituents on the alkyl, alkenyl, alkynyl, or carbocyclyl group of R^{†} are independently, for example, halogen, —R^{•}, -(haloR^{•}), —OH, —OR^{•}, —O(haloR^{•}), —CN, —C(O)OH, —C(O)OR^{•}, —NH₂, —NHR^{•}, —NR^{•}₂, or —NO2, where each R^{•} is unsubstituted or where preceded by “halo” is substituted only with one or more halogens, and is independently C₁₋₆ alkyl, C₁₋₆ alkenyl, C₁₋₆ alkynyl, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

These and other exemplary substituents are described in more detail in the Detailed Description, Examples, and Claims. The invention is not intended to be limited in any manner by the above exemplary listing of substituents.

### Other Definitions

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. Also contemplated are embodiments in which exactly one member of the group is present in, employed in, or otherwise relevant to a given product or process. Also contemplated are embodiments in which more than one, or all of the group members are present in, employed in, or otherwise relevant to a given product or process.

Where the use of the term “about” is before a quantitative value, the present invention also includes the specific quantitative value itself, unless specifically stated otherwise. As used herein, the term “about” refers to a ±10% variation from the nominal value unless otherwise indicated or inferred.

As a general matter, compositions specifying a percentage are by weight unless otherwise specified. Further, if a variable is not accompanied by a definition, then the previous definition of the variable controls.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The terms “pharmaceutically acceptable carrier” and “pharmaceutically acceptable excipient” are used interchangeably herein and refer to buffers, carriers, and excipients suitable for use in contact with the tissues of human beings and animals without excessive toxicity, irritation, allergic response, or other problem or complication, commensurate with a reasonable benefit/risk ratio. Pharmaceutically acceptable carriers include any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers, and adjuvants, see e.g., Adeboye Adejare, Remington: The Science and Practice of Pharmacy (23d ed. 2020).

The term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describes pharmaceutically acceptable salts in detail in J. Pharmaceutical Sciences (1977) 66:1-19, and Gould, Salt selection for basic drugs, International Journal of Pharmaceutics, 33 (1986) 201-217. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Pharmaceutically acceptable salts derived from appropriate bases include alkali metal, alkaline earth metal, ammonium and N+(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

As used herein, a “subject” to which administration is contemplated includes, but is not limited to, humans (e.g., a male or female of any age group, e.g., a pediatric subject (e.g., infant, child, adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal. The terms “human,” “patient,” and “subject” are used interchangeably herein.

Disease, disorder, and condition are used interchangeably herein.

As used herein, “treat”, “treating”, and “treatment” refer to the treatment of a disease, disorder, or symptom or manifestation of such in a subject, e.g., in a human. This includes: (a) preventing a disease or disorder, (b) inhibiting the disease, disorder, etc., i.e., slowing or arresting its progress or development; and (b) relieving the disease, disorder, etc., i.e., causing regression of the disease state. As used herein, “prevent”, “preventing” and “prevention” refer to causing a disease, disorder, or symptom or manifestation of such not to occur for at least a period of time in at least some subjects.

The term “effective amount” as used herein refers to the amount of a compound, e.g., a compound or composition described herein, sufficient to elicit a beneficial or desired result or biological effect. As will be appreciated by those of ordinary skill in this art, the effective amount of a compound or composition described herein may vary depending on such factors as the desired biological endpoint, the pharmacokinetics of the compound, the disease being treated, the mode of administration, and the age, weight, health, and condition of the subject. An effective amount can be administered in one or more administrations, applications, or dosages and is not intended to be limited to a particular formulation or administration route. For example, an effective amount may be an amount of a compound or composition sufficient to achieve one or more of the following: (i) modulate an activity of MAPK signaling pathway, (ii) modulate an activity of a RAF protein (e.g., BRAF protein), (iii) modulate an activity of MEK, (iii) modulate an interaction of RAF and MEK protein complexes, or (iv) treat or prevent a disease or condition associated with MAPK signaling pathway, e.g., cancer, or a symptom or manifestation thereof.

As used herein, and unless otherwise specified, a “therapeutically effective amount” of a compound or composition is an amount sufficient to provide a therapeutic benefit in the treatment of a disease or condition, or to delay or minimize one or more symptoms associated with the disease, disorder or condition. As will be appreciated by those of ordinary skill in this art, a therapeutically effective amount of a compound or composition described herein may vary depending on such factors as the desired biological endpoint, the pharmacokinetics of the compound, the disease being treated, the mode of administration, and the age, weight, health, and condition of the subject. A therapeutically effective amount of a compound or composition can be administered in one or more administrations, applications, or dosages and is not intended to be limited to a particular formulation or administration route. A therapeutically effective amount of a compound or composition also includes an amount of therapeutic agent, alone or in combination with other therapies, which provides a therapeutic benefit in the treatment of the disease, disorder or condition. In addition, a therapeutically effective amount can encompass an amount of a compound or composition that improves overall therapy, reduces or avoids symptoms or causes of disease or condition, or enhances the therapeutic efficacy of another therapeutic agent.

At various places in the present specification, variable or parameters are disclosed in groups or in ranges. It is specifically intended that the description include each and every individual subcombination of the members of such groups and ranges. For example, an integer in the range of 0 to 40 is specifically intended to individually disclose 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, and 40, and an integer in the range of 1 to 20 is specifically intended to individually disclose 1, 2, 3, 4, 5, 6, 7, 8, 9, 10, 11, 12, 13, 14, 15, 16, 17, 18, 19, and 20.

Throughout the description, where compounds or compositions are described as having, including, or comprising specific components, or where processes and methods are described as having, including, or comprising specific steps, it is contemplated that, additionally, there are compounds or compositions of the present invention that consist essentially of, or consist of, the recited components, and that there are processes and methods according to the present invention that consist essentially of, or consist of, the recited processing steps. Similarly, throughout the description, where compounds or compositions are described as consisting essentially of specific components, or where processes and methods are described as consisting essentially of specific steps, it is contemplated that, additionally, there are compounds or compositions of the present invention that consist of the recited components, and that there are processes and methods according to the present invention that consist of the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

It should be understood that the order of steps or order for performing certain actions is immaterial so long as the present invention remain operable. Moreover, two or more steps or actions may be conducted simultaneously.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

The claims encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group.

### II. COMPOUNDS AND COMPOSITIONS

In one aspect, the present disclosure provides a compound of Formula (I):

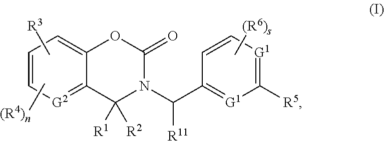

or a pharmaceutically acceptable salt thereof, wherein:
   each G¹ is independently N or CH, wherein the H of CH may be substituted with R⁶;
   G² is N or CH, wherein the H of CH may be substituted with R³ or R⁴;
   R¹ and R² are each independently selected from the group consisting of hydrogen, deuterium, halogen, and C₁₋₆ alkyl, wherein the C₁₋₆ alkyl is optionally substituted, or
   R¹ and R² may be taken together with the carbon atom to which they are attached to form C₃₋₆ cycloalkyl or 3-6 membered heterocyclyl ring, wherein the C₃₋₆ cycloalkyl or 3-6 membered heterocyclyl ring may be optionally substituted;
   R³ is selected from the group consisting of —OR^{•}, —NR^{b}R^{c}, —C(O)N(R^{c})₂, C₁₋₆ alkyl optionally substituted with one or more R⁷, 3-10 membered heterocyclyl, 5-10 membered heteroaryl, C₃₋₁₀ cycloalkyl, and phenyl, wherein the 3-10 membered heterocyclyl, 5-10 membered heteroaryl, C₃₋₁₀ cycloalkyl, and phenyl are optionally substituted;
   each R⁴ is independently selected from the group consisting of hydrogen, halogen, C₁₋₆ alkyl, C₁₋₆ haloalkyl, C₁₋₆ haloalkoxy, —CN, —N(R^{c})₂, NO₂, and C₁₋₆ alkoxy;
   R^{a}, R^{b}, R^{c} and R^{d} are each independently selected from the group consisting of hydrogen, C₁₋₆ alkyl optionally substituted with one or more R⁷, —C(O)-(3-10 membered heterocyclyl or 5-10 membered heteroaryl), —C(O)N(R^{c})₂, —C(O)—(C₁₋₆ alkyl), —C(O)—(C₃₋₁₀ cycloalkyl), 5-10 membered heteroaryl, phenyl, 3-10 membered heterocyclyl, and C₃₋₁₀ cycloalkyl, wherein the 5-10 membered heteroaryl, phenyl, 3-10 membered heterocyclyl, and C₃₋₁₀ cycloalkyl are optionally substituted;
   R⁵ is selected from the group consisting of —(C₁₋₆alkylene)-S(O)ₚR^{h}, —NR^{c}S(O)ₚR^{h}, —NR^{c}C(O)R^{h}, —N(R^{c})₂, NO₂, —OR^{k}, —S(O)_{g}R^{h}, 3-10 membered heterocyclyl, 5-10 membered heteroaryl, (C₁₋₆alkylene)-(3-10 membered heterocyclyl), and (C₁₋₆alkylene)-5-10 membered heteroaryl, wherein the 3-10 membered heterocyclyl and 5-10 membered heteroaryl are optionally substituted;
   each R⁶ is independently selected from the group consisting of C₁₋₆ alkyl, halogen, C₁₋₆ haloalkyl, C₁₋₆ haloalkoxy, —CN, —N(R^{c})₂, NO₂, and C₁₋₆ alkoxy;
   each R⁷ is selected from the group consisting of halogen, —C(O)N(R^{c})₂, —C(O)OR^{p}, 3-10 membered heterocyclyl, and C₃₋₁₀ cycloalkyl, wherein the 3-10 membered heterocyclyl and C₃₋₁₀ cycloalkyl are optionally substituted;
   each R^{e} is independently hydrogen or C₁₋₆ alkyl;
      each R^{h} is independently selected from the group consisting of —NRⁿR^{o}, C₁₋₆ alkyl optionally substituted with one or more R⁸, C₁₋₆ alkoxy, C₃₋₁₀ cycloalkyl, 3-10 membered heterocyclyl, phenyl, and 5-6 membered heteroaryl, wherein the C₃₋₁₀ cycloalkyl, 3-10 membered heterocyclyl, phenyl, and 5-6 membered heteroaryl are optionally substituted;
   R^{k} is selected from the group consisting of C₁₋₆ alkyl optionally substituted with one or more R⁸, C₃₋₁₀ cycloalkyl, 3-10 membered heterocyclyl, phenyl, and 5-6 membered heteroaryl, wherein the C₃₋₁₀ cycloalkyl, 3-10 membered heterocyclyl, phenyl, and 5-6 membered heteroaryl are optionally substituted;
   R⁸ is selected from the group consisting of —N(R^{c})₂, halogen, and C₁₋₆ alkoxy;
   each of Rⁿ and R^{o} is independently selected from hydrogen, and C₁₋₆ alkyl optionally substituted with one or more halogen or C₁₋₆ alkoxy;
   R¹¹ is hydrogen or C₁₋₆ alkyl;
   each R^{p} is independently hydrogen or C₁₋₆ alkyl;
n is selected from 0, 1, 2, and 3;
s is selected from 0, 1, 2, and 3; and
   m, p, and q are each independently selected from 0, 1, and 2; wherein
   when R¹ and R² are each independently hydrogen or optionally substituted C₁₋₆ alkyl, and R⁵ is —NR^{c}S(O)ₚR^{h}, then R^{a} is selected from the group consisting of hydrogen, C₁₋₆ alkyl optionally substituted with one or more R⁷, —C(O)-(3-10 membered heterocyclyl or 5-10 membered heteroaryl), —C(O)—(C₁₋₆ alkyl), —C(O)—(C₃₋₁₀ cycloalkyl), 5-6 membered heteroaryl selected from the group consisting of pyridazinyl, oxazolyl, triazinyl, and pyrazinyl, phenyl, 3-10 membered heterocyclyl, and C₃₋₁₀ cycloalkyl, wherein the 5-6 membered heteroaryl, phenyl, 3-10 membered heterocyclyl, and C₃₋₁₀ cycloalkyl are optionally substituted.

In some embodiments, G¹ is CH. In other embodiments, G¹ is N. In certain embodiments, both G's are CH. In other embodiments, G² is CH. In some embodiments, G² is N.

In certain embodiments, R″ is H. In other embodiments, R¹ is methyl.

In some embodiments, the compound is a compound of formula (I-1):

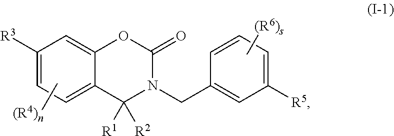

or a pharmaceutically acceptable salt thereof, wherein the variables are as defined herein.

In certain embodiments, the compound is a compound of formula (IA):

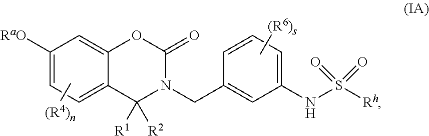

or a pharmaceutically acceptable salt thereof, wherein the variables are as defined herein.

In other embodiments, the compound is a compound of formula (IA-1):

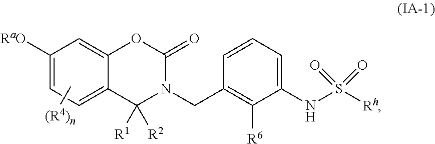

or a pharmaceutically acceptable salt thereof, wherein the variables are as defined herein.

In some embodiments, the compound is a compound of formula (IB):

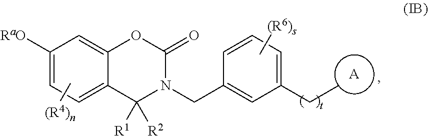

or a pharmaceutically acceptable salt thereof, wherein:
   t is 0 or 1;

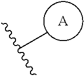

is a 3-10 membered heterocyclyl or 5-10 membered heteroaryl, wherein the 3-10 membered heterocyclyl and 5-10 membered heteroaryl are optionally substituted; and
wherein the remaining variables are as defined herein.

In certain embodiments, the compound is a compound of formula (IB-1):

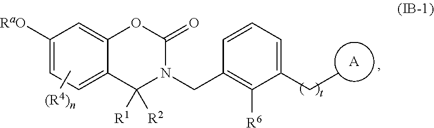

or a pharmaceutically acceptable salt thereof, wherein the variables are as defined herein.

In some embodiments, the compound is a compound of Formula (IC):

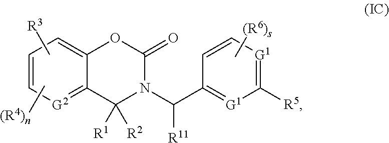

or a pharmaceutically acceptable salt thereof, wherein:
R¹ and R² are taken together with the carbon atom to which they are attached to form C₃₋₆ cycloalkyl or 3-6 membered heterocyclyl ring, wherein the C₃₋₆ cycloalkyl or 3-6 membered heterocyclyl ring may be optionally substituted; and
the remaining variables are as defined herein.

In some embodiments, the compound is a compound of Formula (IC-1):

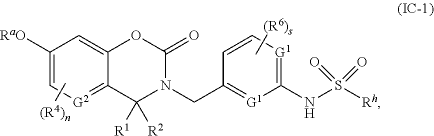

or a pharmaceutically acceptable salt thereof, wherein:
R¹ and R² are taken together with the carbon atom to which they are attached to form C₃₋₆ cycloalkyl or 3-6 membered heterocyclyl ring, wherein the C₃₋₆ cycloalkyl or 3-6 membered heterocyclyl ring may be optionally substituted; and
the remaining variables are as defined herein.

In some embodiments, the compound is a compound of Formula (IC-2):

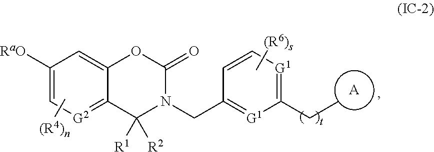

or a pharmaceutically acceptable salt thereof, wherein:
R¹ and R² are taken together with the carbon atom to which they are attached to form C₃ 6 cycloalkyl or 3-6 membered heterocyclyl ring, wherein the C₃₋₆ cycloalkyl or 3-6 membered heterocyclyl ring may be optionally substituted; and
the remaining variables are as defined herein.

In certain embodiments, the compound is a compound of Formula (ID):

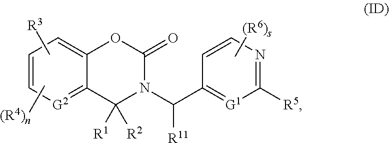

or a pharmaceutically acceptable salt thereof, wherein the variables are as defined herein.

In some embodiments, the compound is a compound of Formula (IE):

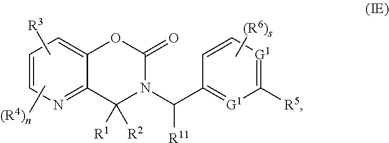

or a pharmaceutically acceptable salt thereof, wherein the variables are as defined herein.

In certain embodiments, the compound is a compound of Formula (IF):

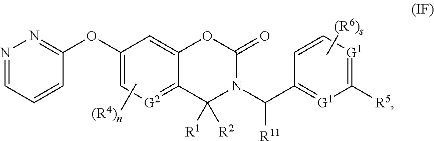

or a pharmaceutically acceptable salt thereof, wherein the variables are as defined herein.

In some embodiments, the compound is a compound of Formula (IF-1):

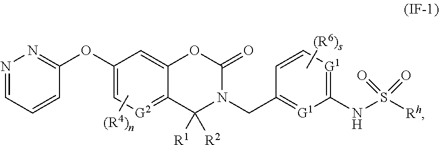

or a pharmaceutically acceptable salt thereof, wherein the variables are as defined herein.

In some embodiments, the compound is a compound of Formula (IF-2):

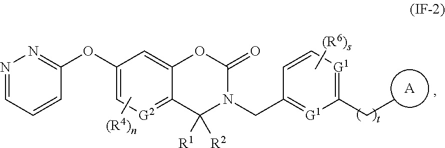

or a pharmaceutically acceptable salt thereof, wherein the variables are as defined herein.

In some embodiments, the compound is a compound of Formula (IG):

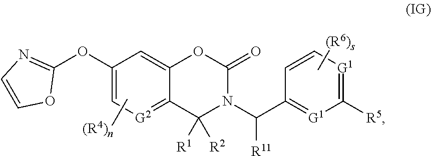

or a pharmaceutically acceptable salt thereof, wherein the variables are as defined herein.

In some embodiments, the compound is a compound of Formula (IG-1):

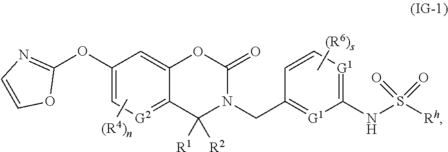

or a pharmaceutically acceptable salt thereof, wherein the variables are as defined herein.

In some embodiments, the compound is a compound of Formula (IG-2):

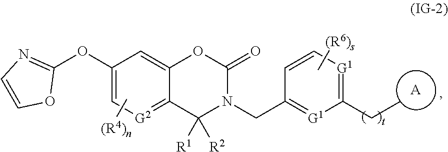

or a pharmaceutically acceptable salt thereof, wherein the variables are as defined herein.

In some embodiments, each G¹ is CH, wherein the H of CH may be substituted with R⁶. In some embodiments, one G¹ is N and the other G¹ is CH, wherein the H of CH may be substituted with R⁶. In some embodiments, G¹ is CH, wherein the H of CH may be substituted with R⁶. In some embodiments, G¹ is N.

In some embodiments, G² is N. In some embodiments, G² is CH, wherein the H of CH may be substituted with R³ or R⁴.

In some embodiments, R¹ and R² are each independently selected from the group consisting of hydrogen, deuterium, halogen, and C₁₋₆ alkyl, wherein the C₁₋₆ alkyl is optionally substituted with one, two, or three halogen(s).

In certain embodiments, R¹ and R² may be taken together with the carbon atom to which they are attached to form C₃₋₆ cycloalkyl or 3-6 membered heterocyclyl ring, wherein the C₃₋₆ cycloalkyl or 3-6 membered heterocyclyl ring may be optionally substituted with C₁₋₆ alkyl.

In some embodiments, R¹ and R² are each independently selected from the group consisting of hydrogen, deuterium, halogen, methyl, and —CF₃.

In certain embodiments, R¹ and R² are taken together with the carbon atom to which they are attached to form cyclobutyl ring, oxetanyl ring, or azetidinyl ring, wherein the cyclobutyl ring, oxetanyl ring, or azetidinyl ring is optionally substituted with C₁₋₆ alkyl. In some embodiments, R¹ and R² are taken together with the carbon atom to which they are attached to form cyclopropyl ring, cyclobutyl ring, oxetanyl ring, or azetidinyl ring, wherein the cyclopropyl ring, cyclobutyl ring, oxetanyl ring, or azetidinyl ring is optionally substituted with C₁₋₆ alkyl.

In other embodiments, R³ is selected from the group consisting of —OR^{•}, —NR^{b}R^{c}, —S(O)ₘR^{d}, —C(O)N(R^{c})₂, C₁₋₆ alkyl optionally substituted with one or more R⁷, 3-10 membered heterocyclyl, 5-10 membered heteroaryl, C₃₋₁₀ cycloalkyl, and phenyl, wherein the 3-10 membered heterocyclyl and C₃₋₁₀ cycloalkyl are optionally substituted with one or two oxo.

In some embodiments, R³ is selected from the group consisting of —OR^{a}, C₁₋₆ alkylene optionally substituted with one or more R⁷, —NR^{b}R^{c}, —C(O)N(R^{c})₂, 5-10 membered heteroaryl, and 3-10 membered heterocyclyl that is optionally substituted with one or two oxo.

In other embodiments, R^{b} is selected from —C(O)-(3-10 membered heterocyclyl or 5-10 membered heteroaryl) and —C(O)N(R^{c})₂, and R^{o} is selected from hydrogen and C₁₋₆ alkyl.

In other embodiments, R³ is —OR^{a} or C₁₋₆ alkylene optionally substituted with one or more R⁷.

In some embodiments, R³ is —OR^{a}.

In other embodiments, each of R^{a}, R^{b}, R^{c} and R^{d} is selected from the group consisting of hydrogen, C₁₋₆ alkyl optionally substituted with one or more R⁷, —C(O)-(3-10 membered heterocyclyl or 5-10 membered heteroaryl), —C(O)N(R^{c})₂, —C(O)—(C₁₋₆ alkyl), —C(O)—(C₃₋₁₀ cycloalkyl), 5-10 membered heteroaryl, phenyl, 3-10 membered heterocyclyl, and C₃₋₁₀ cycloalkyl, wherein the −10 membered heteroaryl, phenyl, 3-10 membered heterocyclyl, and C₃₋₁₀ cycloalkyl are optionally substituted with one, two, or three C₁₋₆ alkyl.

In some embodiments, R^{a} is selected from the group consisting of C(O)N(R^{c})₂, 5-10 membered heteroaryl, C₁₋₆ alkyl optionally substituted with one or more R⁷, C₃₋₁₀ cycloalkyl, —C(O)-(3-10 membered heterocyclyl), —C(O)—(C₁₋₆ alkyl), and —C(O)—(C₃₋₁₀ cycloalkyl).

In some embodiments, R^{a} is selected from the group consisting of C(O)N(R^{o})₂, 5-6 membered heteroaryl optionally substituted with C₁₋₆ alkyl, C₁₋₆ alkyl optionally substituted with one or more R⁷, C₃₋₁₀ cycloalkyl, —C(O)-(3-10 membered heterocyclyl), —C(O)—(C₁₋₆ alkyl), and —C(O)—(C₃₋₁₀ cycloalkyl), wherein the 5-6 membered heteroaryl is selected from the group consisting of pyridazinyl, oxazolyl, triazinyl, and pyrazinyl.

In other embodiments, R^{a} is 5-6 membered heteroaryl selected from the group consisting of pyridazinyl, oxazolyl, triazinyl, and pyrazinyl, wherein the 5-6 membered heteroaryl is optionally substituted with C₁₋₆ alkyl.

In certain embodiments, R^{a} is C(O)N(R^{c})₂ or 5-10 membered heteroaryl.

In other embodiments, R^{a} is 5-6 membered heteroaryl.

In some embodiments, R^{a} is a 6-membered heteroaryl having two or three nitrogen.

In certain embodiments, R^{a} is selected from the group consisting of:

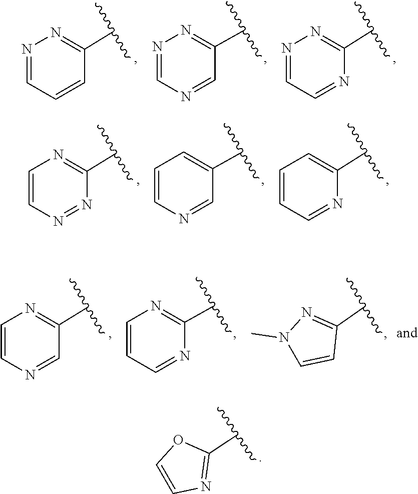

In some embodiments, R^{a} is selected from the group consisting of:

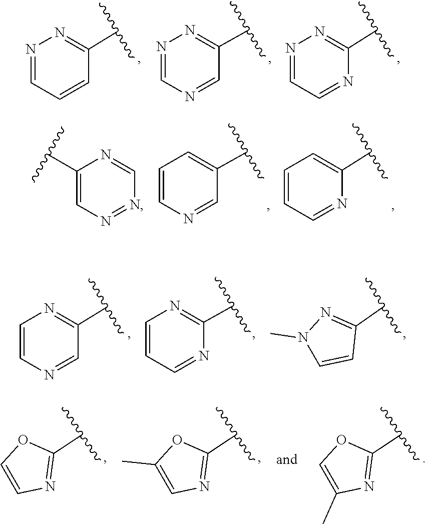

In other embodiments, R^{a} is selected from the group consisting of:

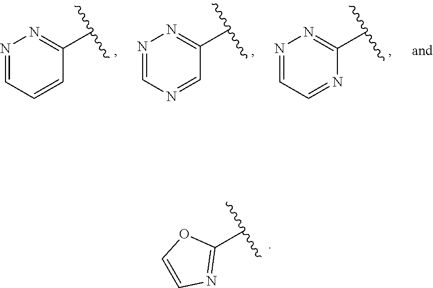 [SMILES: *c1cccnn1 ; *c1cncnn1 ; *c1nccnn1 ; *c1ncco1]

In some embodiments, R^{a} is selected from the group consisting of:

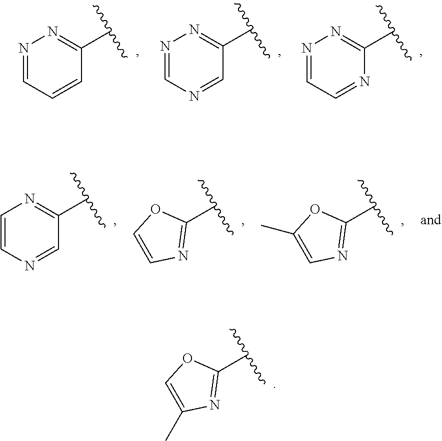

In some embodiments, R^{a} is selected from:

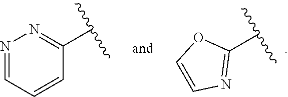 [SMILES: *c1cccnn1 ; *c1ncco1]

In some embodiments, R^{a} is

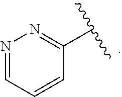 [SMILES: *c1cccnn1]

In other embodiments, R^{a} is:

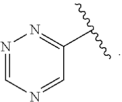 [SMILES: *c1cncnn1]

In certain embodiments, R^{a} is:

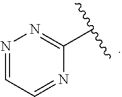 [SMILES: *c1nccnn1]

In some embodiments, R^{a} is:

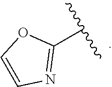 [SMILES: *c1ncco1]

In some embodiments, R^{a} is:

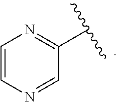 [SMILES: *c1cnccn1]

In some embodiments, R^{a} is:

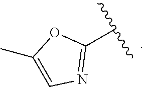 [SMILES: *c1ncc(C)o1]

In some embodiments, R″ is:

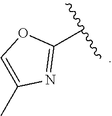 [SMILES: *c1nc(C)co1]

In some embodiments, each R⁷ is independently selected from the group consisting of halogen, —C(O)N(R^{c})₂, —C(O)OR^{p}, 3-10 membered heterocyclyl, and C₃₋₁₀ cycloalkyl, wherein the 3-10 membered heterocyclyl and C₃₋₁₀ cycloalkyl are optionally substituted with one or two oxo.

In certain embodiments, each R⁷ is independently selected from the group consisting of halogen, —C(O)N(R^{c})₂, 3-10 membered heterocyclyl, and C₃₋₁₀ cycloalkyl.

In other embodiments, each R⁷ is independently halogen or —C(O)N(R^{c})₂.

In certain embodiments, each R^{•} is hydrogen or C₁₋₆ alkyl.

In some embodiments, each R^{e} is C₁₋₆ alkyl.

In certain embodiments, each R⁴ is selected from the group consisting of halogen, C₁₋₆ alkyl, and C₁₋₆ haloalkyl.

In certain embodiments, each R⁴ is halogen.

In some embodiments, R⁵ is selected from the group consisting of —(C₁₋₆ alkylene)-S(O)ₚR^{h}, —NR^{c}S(O)ₚR^{h}, —NR^{c}C(O)R^{h}, —N(R^{c})₂, NO₂, —OR^{k}, —S(O)_{q}R^{h}, 3-10 membered heterocyclyl, 5-10 membered heteroaryl, (C₁₋₆ alkylene)-(3-10 membered heterocyclyl), and (C₁₋₆ alkylene)-5-10 membered heteroaryl, wherein the 3-10 membered heterocyclyl is optionally substituted with one or two oxo.

In other embodiments, R⁵ is selected from the group consisting of NR^{c}S(O)ₚR^{h}, NR^{c}C(O)R^{h}, and —(C₁₋₆ alkylene)-S(O)ₚR^{h}.

In other embodiments, R⁵ is NR^{c}S(O)ₚR^{h}, for example, NHS(O)₂R^{h}.

In certain embodiments, each R^{h} is independently selected from the group consisting of —NRⁿR^{o}, C₁₋₆ alkoxy, C₁₋₆ alkyl optionally substituted with one or more R⁸, C₁₋₆ alkoxy, C₃₋₁₀ cycloalkyl, 3-10 membered heterocyclyl, phenyl, and 5-6 membered heteroaryl, wherein the C₃₋₁₀ cycloalkyl, 3-10 membered heterocyclyl, phenyl, and 5-6 membered heteroaryl are optionally substituted with one or more substituents (e.g., one, two, three, or four substituents) each independently selected from the group consisting of halogen, C₁₋₆ alkyl, C₁₋₆ haloalkyl, C₁₋₆ haloalkoxy, C₁₋₆ alkoxy, and C(O)NR^{p}R^{q}, wherein R^{p} and R are independently hydrogen or C₁₋₆ alkyl.

In some embodiments, R^{h} is selected from the group consisting of —NRⁿR^{o}, C₃₋₁₀ cycloalkyl, and 3-10 membered heterocyclyl, wherein the C₃₋₁₀ cycloalkyl, 3-10 membered heterocyclyl, and 5-6 membered heteroaryl are optionally substituted with one or more substituents (e.g., one, two, three, or four substituents) each independently selected from the group consisting of halogen, C₁₋₆ alkyl, C₁₋₆ haloalkyl, C₁₋₆ haloalkoxy, C₁₋₆ alkoxy, and C(O)NR^{p}Rⁿ.

In certain embodiments, R^{h} is —NRⁿR^{o}. In some embodiments, R^{h} is —NHCH₃.

In some embodiments, each R″ and R^{o} are independently selected from hydrogen, methy, and —C₁₋₆alkylene-C₁₋₆alkoxy.

In other embodiments, each R″ and R^{o} are independently selected from hydrogen and methyl.

In some embodiments, R^{h} is 3-10 membered heterocyclyl optionally substituted with one or more substituents independently selected from the group consisting of halogen, C₁₋₆ alkyl, C₁₋₆ haloalkyl, C₁₋₆ haloalkoxy, C₁₋₆ alkoxy, and C(O)NR^{p}R^{q}.

In other embodiments, R^{k} is selected from the group consisting of —NRⁿR^{o}, C₁₋₆ alkyl optionally substituted with one or more R⁸, C₃₋₁₀ cycloalkyl, 3-10 membered heterocyclyl, phenyl, and 5-6 membered heteroaryl, wherein the C₃₋₁₀ cycloalkyl, 3-10 membered heterocyclyl, phenyl, and 5-6 membered heteroaryl are optionally substituted with one, two, or three substituents each independently selected from halogen and C₁₋₆ alkyl.

In certain embodiments, R⁵ is selected from the group consisting of 3-10 membered heterocyclyl, 5-10 membered heteroaryl, (C₁₋₆ alkylene)-3-10 membered heterocyclyl, and (C₁₋₆ alkylene)-5-10 membered heteroaryl wherein the 3-10 membered heterocyclyl is optionally substituted with one or two oxo.

In other embodiments, R⁵ is selected from the group consisting of −3-10 membered heterocyclyl and (C₁₋₆ alkylene)-3-10 membered heterocyclyl, wherein the 3-10 membered heterocyclyl is optionally substituted with oxo.

In some embodiments, each R⁶ is halogen. In some embodiments, R⁶ is chloro. In some embodiments, R⁶ is fluoro. In some embodiments, R⁶ is vicinal to the two substituents on the aryl group.

In certain embodiments, n is 0. In other embodiments, n is 1.

In some embodiments, s is 0. In certain embodiments, s is 1. In other embodiments, s is 2.

In other embodiments, t is 0. In some embodiments, t is 1.

In certain embodiments,

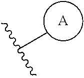

is a 3-10 membered heterocyclyl wherein the 3-10 membered heterocyclyl is optionally substituted.

In some embodiments,

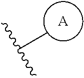

is selected from the group consisting of

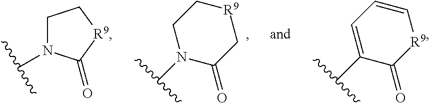

and wherein R⁹ is selected from the group consisting of O, NH, N—C₁₋₆ alkyl, N—C₁₋₆ alkoxy, and N—(C₁₋₆ alkylene)-C(O)N(R^{c})₂.

In some embodiments,

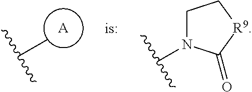

In some embodiments,

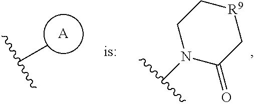

wherein R⁹ is O.

In some embodiments,

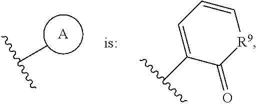

wherein R⁹ is NH.

In certain embodiments, the compound is selected from the compound of Table 1 or a pharmaceutically acceptable salt thereof.

TABLE 1
List of exemplified compounds

| Compound No. | Structure |
|---|---|
| 1 | 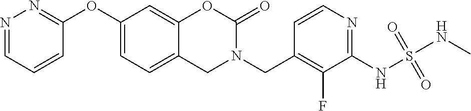 [SMILES: CNS(=O)(=O)Nc1nccc(CN2Cc3ccc(Oc4cccnn4)cc3OC2=O)c1F] |
| 2 | 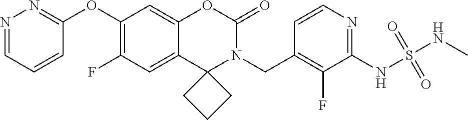 [SMILES: CNS(=O)(=O)Nc1nccc(CN2C(=O)Oc3cc(Oc4cccnn4)c(F)cc3C23CCC3)c1F] |
| 3 | 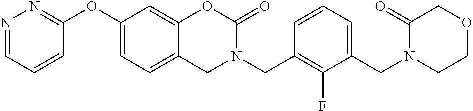 [SMILES: O=C1COCCN1Cc1cccc(CN2Cc3ccc(Oc4cccnn4)cc3OC2=O)c1F] |
| 4 | 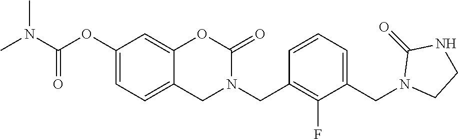 [SMILES: CN(C)C(=O)Oc1ccc2c(c1)OC(=O)N(Cc1cccc(CN3CCNC3=O)c1F)C2] |
| 5 | 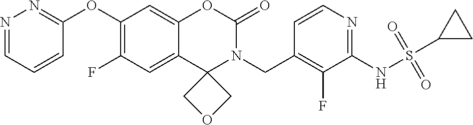 [SMILES: O=C1Oc2cc(Oc3cccnn3)c(F)cc2C2(COC2)N1Cc1ccnc(NS(=O)(=O)C2CC2)c1F] |
| 6 | 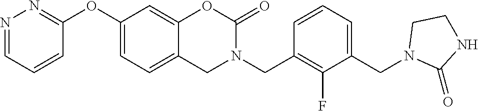 [SMILES: O=C1NCCN1Cc1cccc(CN2Cc3ccc(Oc4cccnn4)cc3OC2=O)c1F] |
| 7 | 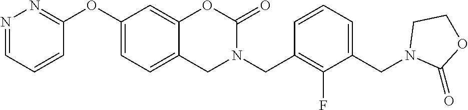 [SMILES: O=C1OCCN1Cc1cccc(CN2Cc3ccc(Oc4cccnn4)cc3OC2=O)c1F] |
| 8 | 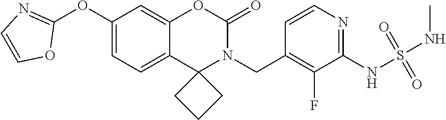 [SMILES: CNS(=O)(=O)Nc1nccc(CN2C(=O)Oc3cc(Oc4ncco4)ccc3C23CCC3)c1F] |
| 9 | 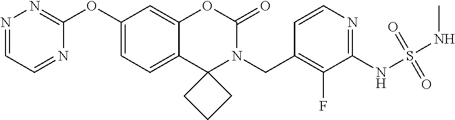 [SMILES: CNS(=O)(=O)Nc1nccc(CN2C(=O)Oc3cc(Oc4nccnn4)ccc3C23CCC3)c1F] |
| 10 | 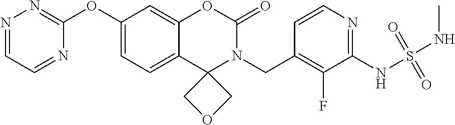 [SMILES: CNS(=O)(=O)Nc1nccc(CN2C(=O)Oc3cc(Oc4nccnn4)ccc3C23COC3)c1F] |
| 11 | 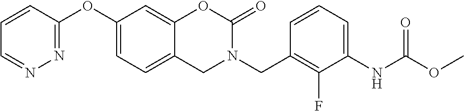 [SMILES: COC(=O)Nc1cccc(CN2Cc3ccc(Oc4cccnn4)cc3OC2=O)c1F] |
| 12 | 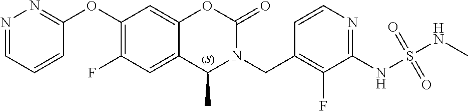 |
| | or |
| | 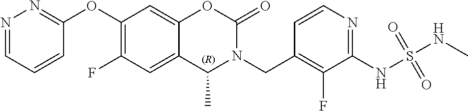 |
| 13 | 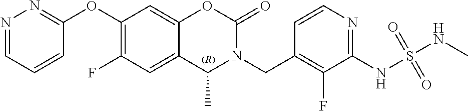 |
| | or |
| | 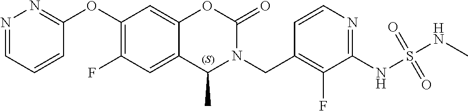 |
| 14 | 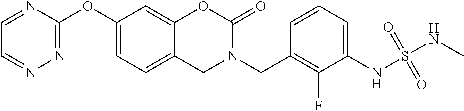 [SMILES: CNS(=O)(=O)Nc1cccc(CN2Cc3ccc(Oc4nccnn4)cc3OC2=O)c1F] |
| 15 | 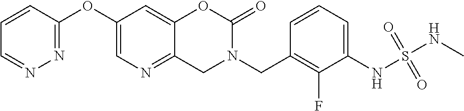 [SMILES: CNS(=O)(=O)Nc1cccc(CN2Cc3ncc(Oc4cccnn4)cc3OC2=O)c1F] |
| 16 | 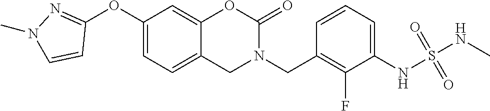 |
| 17 | 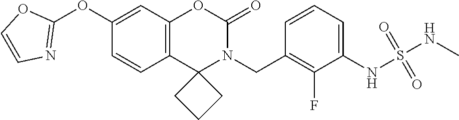 [SMILES: CNS(=O)(=O)Nc1cccc(CN2C(=O)Oc3cc(Oc4ncco4)ccc3C23CCC3)c1F] |
| 18 | 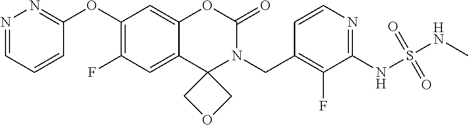 [SMILES: CNS(=O)(=O)Nc1nccc(CN2C(=O)Oc3cc(Oc4cccnn4)c(F)cc3C23COC3)c1F] |
| 19 | 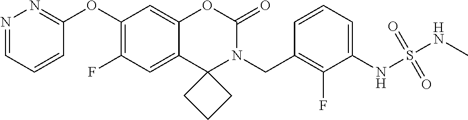 [SMILES: CNS(=O)(=O)Nc1cccc(CN2C(=O)Oc3cc(Oc4cccnn4)c(F)cc3C23CCC3)c1F] |
| 20 | 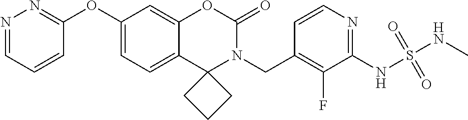 [SMILES: CNS(=O)(=O)Nc1nccc(CN2C(=O)Oc3cc(Oc4cccnn4)ccc3C23CCC3)c1F] |
| 21 | 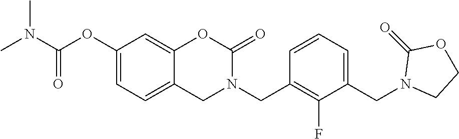 [SMILES: CN(C)C(=O)Oc1ccc2c(c1)OC(=O)N(Cc1cccc(CN3CCOC3=O)c1F)C2] |
| 22 | 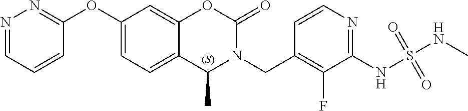 |
| | or |
| | 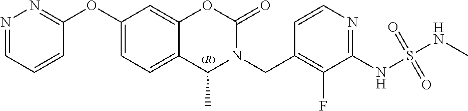 |
| 23 | 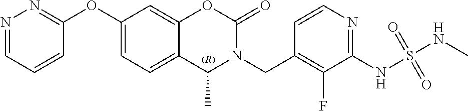 |
| | or |
| | 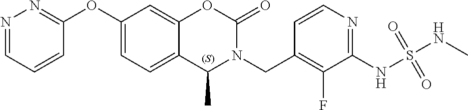 |
| 24 | 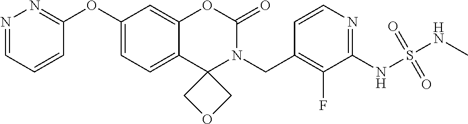 [SMILES: CNS(=O)(=O)Nc1nccc(CN2C(=O)Oc3cc(Oc4cccnn4)ccc3C23COC3)c1F] |
| 25 | 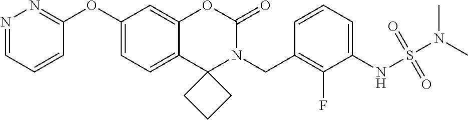 [SMILES: CN(C)S(=O)(=O)Nc1cccc(CN2C(=O)Oc3cc(Oc4cccnn4)ccc3C23CCC3)c1F] |
| 26 | 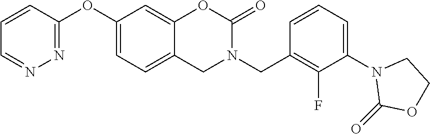 [SMILES: O=C1Oc2cc(Oc3cccnn3)ccc2CN1Cc1cccc(N2CCOC2=O)c1F] |
| 27 | 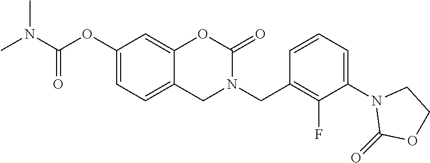 [SMILES: CN(C)C(=O)Oc1ccc2c(c1)OC(=O)N(Cc1cccc(N3CCOC3=O)c1F)C2] |
| 28 | 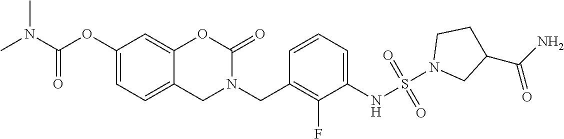 [SMILES: CN(C)C(=O)Oc1ccc2c(c1)OC(=O)N(Cc1cccc(NS(=O)(=O)N3CCC(C(N)=O)C3)c1F)C2] |
| 29 | 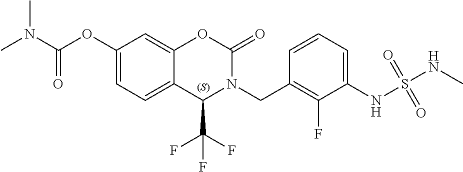 |
| | or |
| | 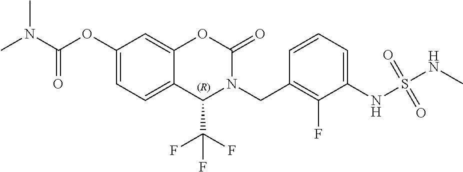 |
| 30 | 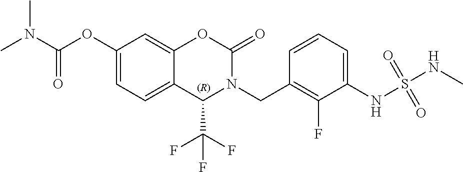 |
| | or |
| | 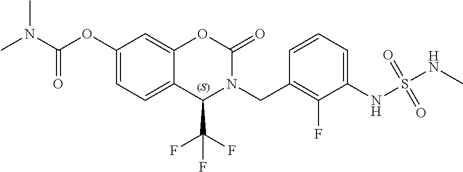 |
| 31 | 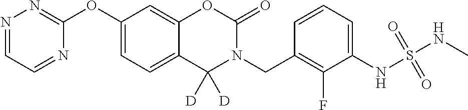 [SMILES: [2H]C1([2H])c2ccc(Oc3nccnn3)cc2OC(=O)N1Cc1cccc(NS(=O)(=O)NC)c1F] |
| 32 | 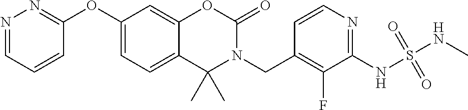 [SMILES: CNS(=O)(=O)Nc1nccc(CN2C(=O)Oc3cc(Oc4cccnn4)ccc3C2(C)C)c1F] |
| 33 | 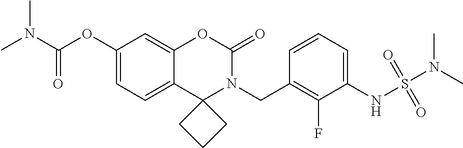 |
| 34 | 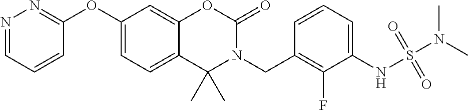 [SMILES: CN(C)S(=O)(=O)Nc1cccc(CN2C(=O)Oc3cc(Oc4cccnn4)ccc3C2(C)C)c1F] |
| 35 | 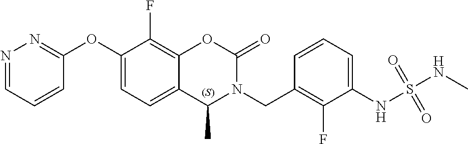 |
| | or |
| | 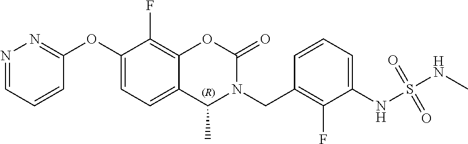 |
| 36 | 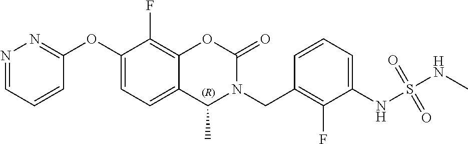 |
| | or |
| | 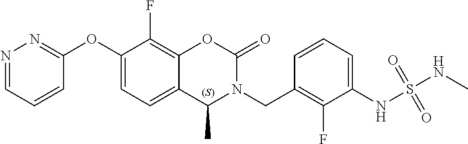 |
| 37 | 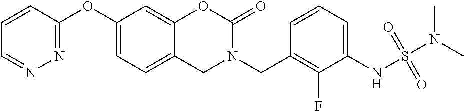 [SMILES: CN(C)S(=O)(=O)Nc1cccc(CN2Cc3ccc(Oc4cccnn4)cc3OC2=O)c1F] |
| 38 | 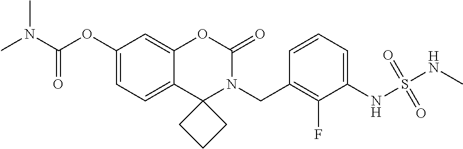 |
| 39 | 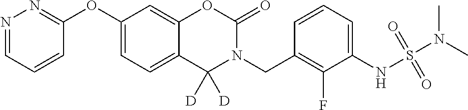 [SMILES: [2H]C1([2H])c2ccc(Oc3cccnn3)cc2OC(=O)N1Cc1cccc(NS(=O)(=O)N(C)C)c1F] |
| 40 | 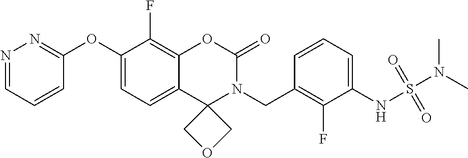 [SMILES: CN(C)S(=O)(=O)Nc1cccc(CN2C(=O)Oc3c(ccc(Oc4cccnn4)c3F)C23COC3)c1F] |
| 41 | 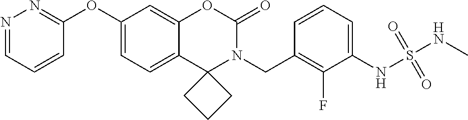 [SMILES: CNS(=O)(=O)Nc1cccc(CN2C(=O)Oc3cc(Oc4cccnn4)ccc3C23CCC3)c1F] |
| 42 | 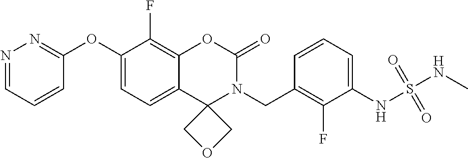 [SMILES: CNS(=O)(=O)Nc1cccc(CN2C(=O)Oc3c(ccc(Oc4cccnn4)c3F)C23COC3)c1F] |
| 43 | 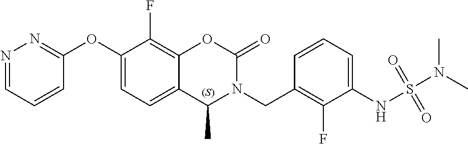 |
| | or |
| | 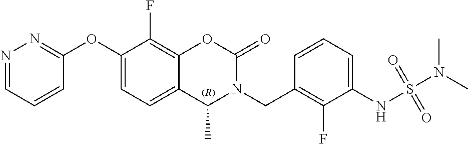 |
| 44 | 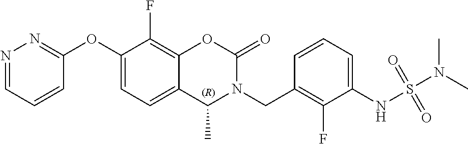 |
| | or |
| | 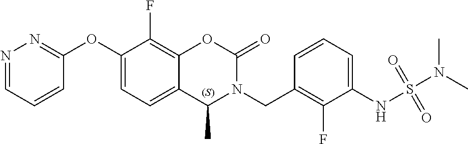 |
| 45 | 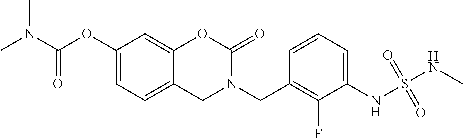 |
| 46 | 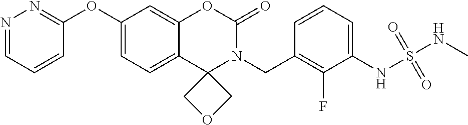 [SMILES: CNS(=O)(=O)Nc1cccc(CN2C(=O)Oc3cc(Oc4cccnn4)ccc3C23COC3)c1F] |
| 47 | 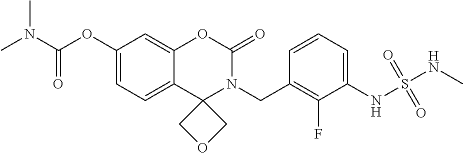 |
| 48 | 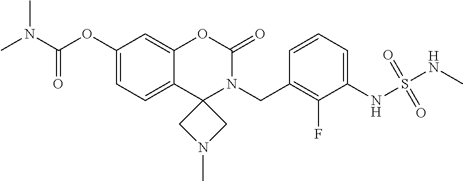 |
| 49 | 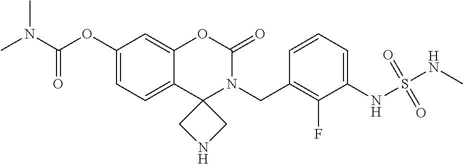 |
| 50 | 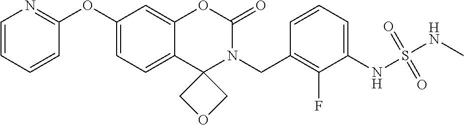 [SMILES: CNS(=O)(=O)Nc1cccc(CN2C(=O)Oc3cc(Oc4ccccn4)ccc3C23COC3)c1F] |
| 51 | 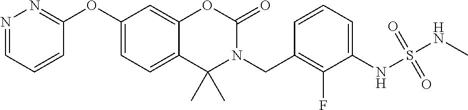 [SMILES: CNS(=O)(=O)Nc1cccc(CN2C(=O)Oc3cc(Oc4cccnn4)ccc3C2(C)C)c1F] |
| 52 | 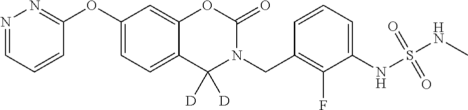 [SMILES: [2H]C1([2H])c2ccc(Oc3cccnn3)cc2OC(=O)N1Cc1cccc(NS(=O)(=O)NC)c1F] |
| 53 | 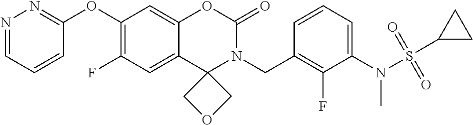 [SMILES: CN(c1cccc(CN2C(=O)Oc3cc(Oc4cccnn4)c(F)cc3C23COC3)c1F)S(=O)(=O)C1CC1] |
| 54 | 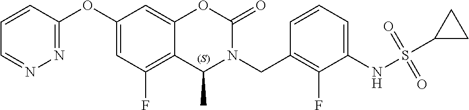 |
| | or |
| | 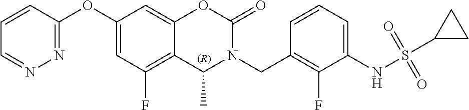 |
| 55 | 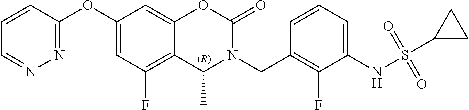 |
| | or |
| | 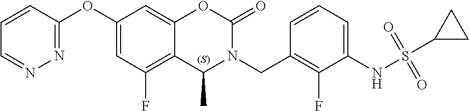 |
| 56 | 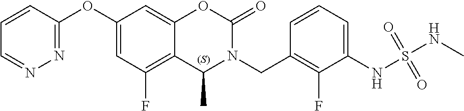 |
| | or |
| | 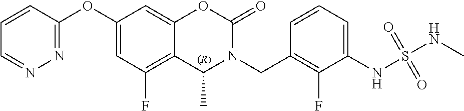 |
| 57 | 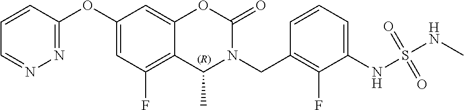 |
| | or |
| | 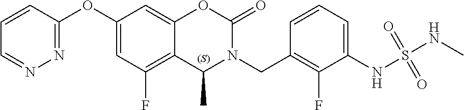 |
| 58 | 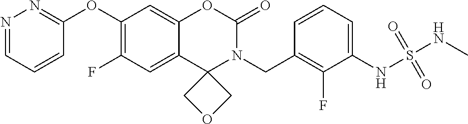 [SMILES: CNS(=O)(=O)Nc1cccc(CN2C(=O)Oc3cc(Oc4cccnn4)c(F)cc3C23COC3)c1F] |
| 59 | 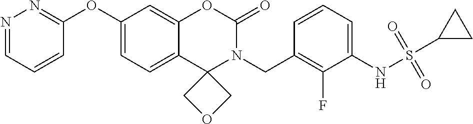 [SMILES: O=C1Oc2cc(Oc3cccnn3)ccc2C2(COC2)N1Cc1cccc(NS(=O)(=O)C2CC2)c1F] |
| 60 | 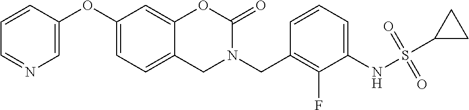 [SMILES: O=C1Oc2cc(Oc3cccnc3)ccc2CN1Cc1cccc(NS(=O)(=O)C2CC2)c1F] |
| 61 | 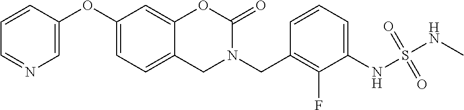 [SMILES: CNS(=O)(=O)Nc1cccc(CN2Cc3ccc(Oc4cccnc4)cc3OC2=O)c1F] |
| 62 | 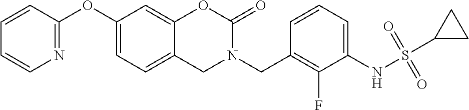 [SMILES: O=C1Oc2cc(Oc3ccccn3)ccc2CN1Cc1cccc(NS(=O)(=O)C2CC2)c1F] |
| 63 | 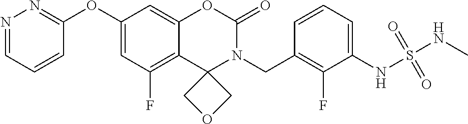 [SMILES: CNS(=O)(=O)Nc1cccc(CN2C(=O)Oc3cc(Oc4cccnn4)cc(F)c3C23COC3)c1F] |
| 64 | 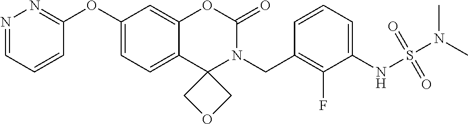 [SMILES: CN(C)S(=O)(=O)Nc1cccc(CN2C(=O)Oc3cc(Oc4cccnn4)ccc3C23COC3)c1F] |
| 65 | 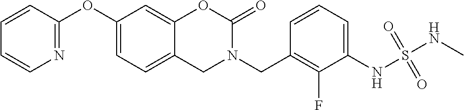 [SMILES: CNS(=O)(=O)Nc1cccc(CN2Cc3ccc(Oc4ccccn4)cc3OC2=O)c1F] |
| 66 | 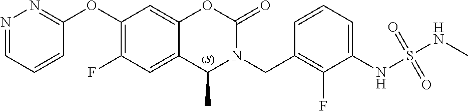 |
| | or |
| | 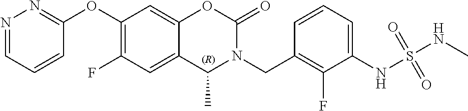 |
| 67 | 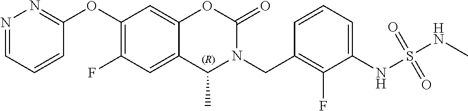 |
| | or |
| | 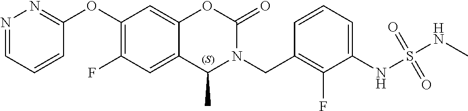 |
| 68 | 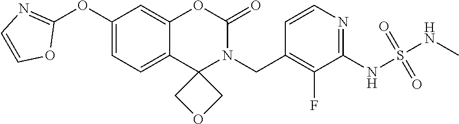 [SMILES: CNS(=O)(=O)Nc1nccc(CN2C(=O)Oc3cc(Oc4ncco4)ccc3C23COC3)c1F] |
| 69 | 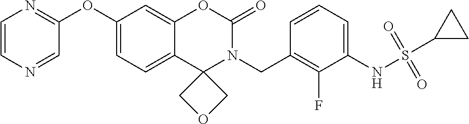 [SMILES: O=C1Oc2cc(Oc3cnccn3)ccc2C2(COC2)N1Cc1cccc(NS(=O)(=O)C2CC2)c1F] |
| 70 | 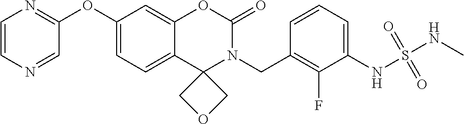 [SMILES: CNS(=O)(=O)Nc1cccc(CN2C(=O)Oc3cc(Oc4cnccn4)ccc3C23COC3)c1F] |
| 71 | 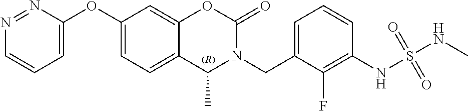 |
| | or |
| | 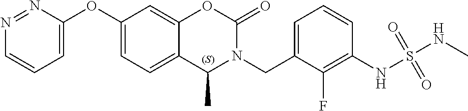 |
| 72 | 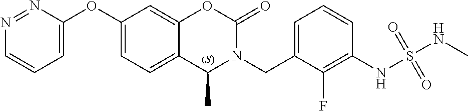 |
| | or |
| | 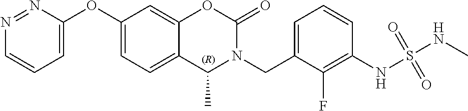 |
| 73 | 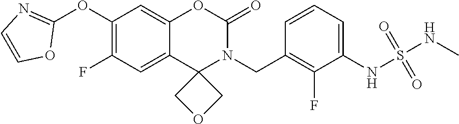 [SMILES: CNS(=O)(=O)Nc1cccc(CN2C(=O)Oc3cc(Oc4ncco4)c(F)cc3C23COC3)c1F] |
| 74 | 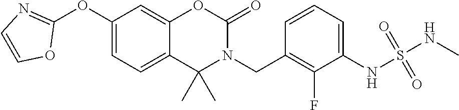 [SMILES: CNS(=O)(=O)Nc1cccc(CN2C(=O)Oc3cc(Oc4ncco4)ccc3C2(C)C)c1F] |
| 75 | 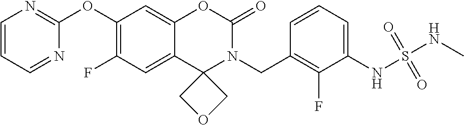 [SMILES: CNS(=O)(=O)Nc1cccc(CN2C(=O)Oc3cc(Oc4ncccn4)c(F)cc3C23COC3)c1F] |
| 76 | 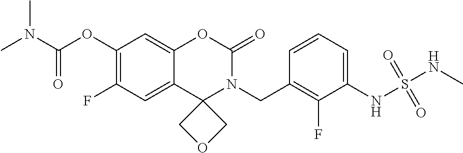 |
| 77 | 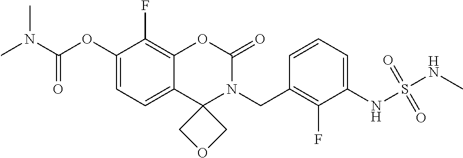 |
| 78 | 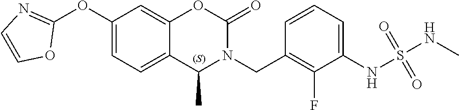 |
| | or |
| | 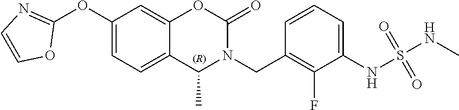 |
| 79 | 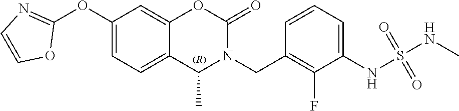 |
| | or |
| | 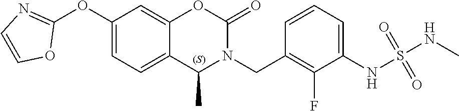 |
| 80 | 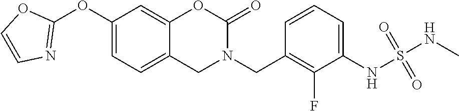 [SMILES: CNS(=O)(=O)Nc1cccc(CN2Cc3ccc(Oc4ncco4)cc3OC2=O)c1F] |
| 81 | 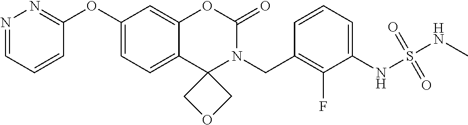 [SMILES: CNS(=O)(=O)Nc1cccc(CN2C(=O)Oc3cc(Oc4cccnn4)ccc3C23COC3)c1F] |
| 82 | 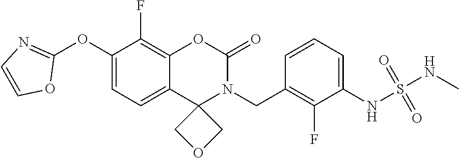 [SMILES: CNS(=O)(=O)Nc1cccc(CN2C(=O)Oc3c(ccc(Oc4ncco4)c3F)C23COC3)c1F] |
| 83 | 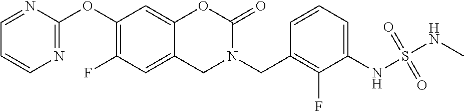 [SMILES: CNS(=O)(=O)Nc1cccc(CN2Cc3cc(F)c(Oc4ncccn4)cc3OC2=O)c1F] |
| 84 | 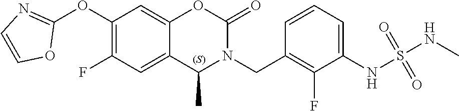 |
| | or |
| | 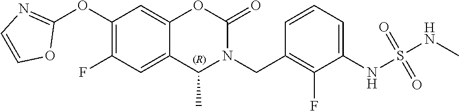 |
| 85 | 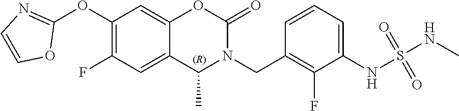 |
| | or |
| | 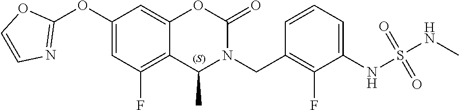 |
| 86 | 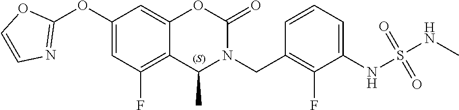 |
| | or |
| | 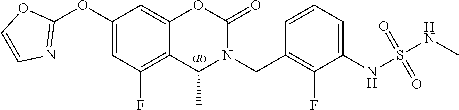 |
| 87 | 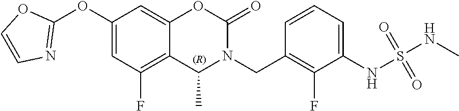 |
| | or |
| | 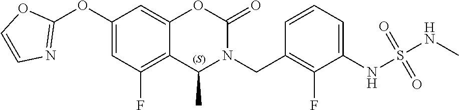 |
| 88 | 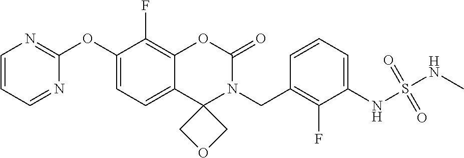 [SMILES: CNS(=O)(=O)Nc1cccc(CN2C(=O)Oc3c(ccc(Oc4ncccn4)c3F)C23COC3)c1F] |
| 89 | 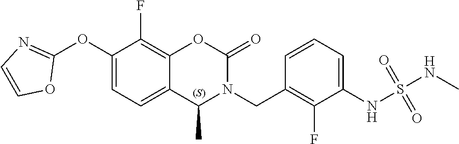 |
| | or |
| | 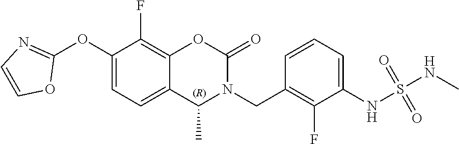 |
| 90 | 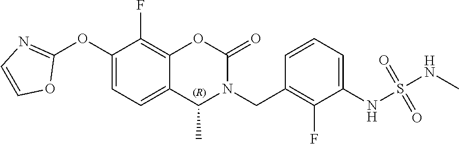 |
| | or |
| | 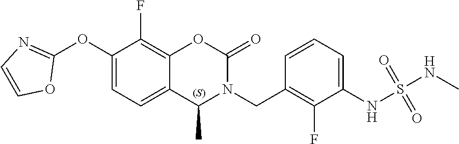 |
| 91 | 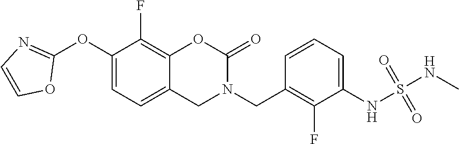 [SMILES: CNS(=O)(=O)Nc1cccc(CN2Cc3ccc(Oc4ncco4)c(F)c3OC2=O)c1F] |
| 92 | 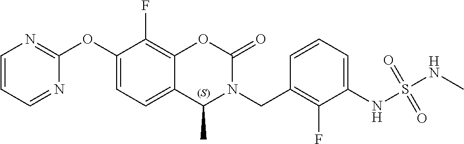 |
| | or |
| | 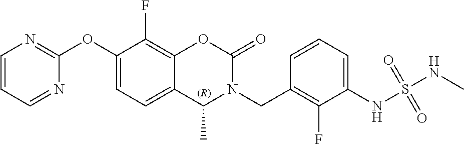 |
| 93 | 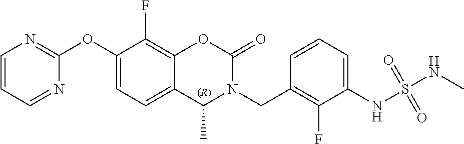 |
| | or |
| | 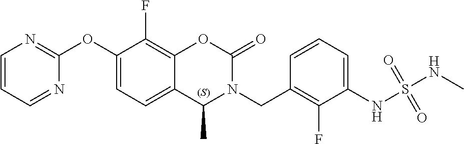 |
| 94 | 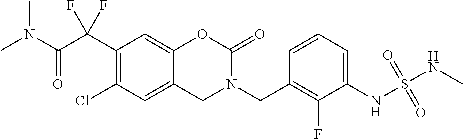 |
| 95 | 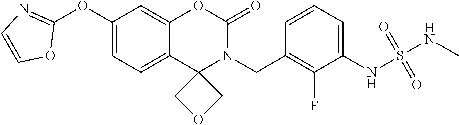 [SMILES: CNS(=O)(=O)Nc1cccc(CN2C(=O)Oc3cc(Oc4ncco4)ccc3C23COC3)c1F] |
| 96 | 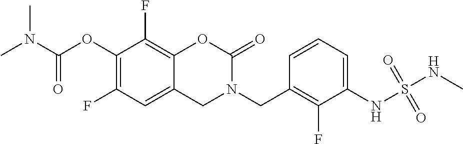 |
| 97 | 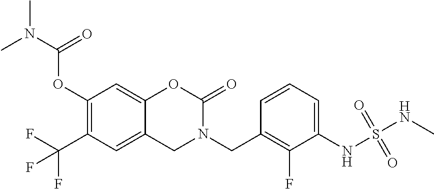 |
| 98 | 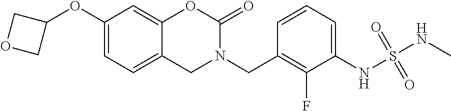 [SMILES: CNS(=O)(=O)Nc1cccc(CN2Cc3ccc(OC4COC4)cc3OC2=O)c1F] |
| 99 | 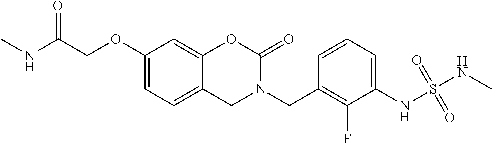 |
| 100 | 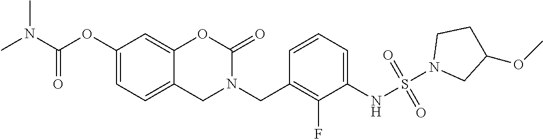 |
| 101 | 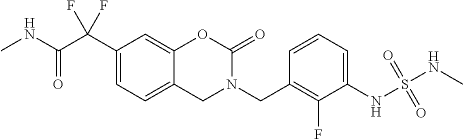 |
| 102 | 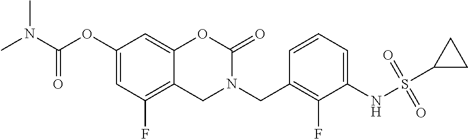 [SMILES: CN(C)C(=O)Oc1cc(F)c2c(c1)OC(=O)N(Cc1cccc(NS(=O)(=O)C3CC3)c1F)C2] |
| 103 | 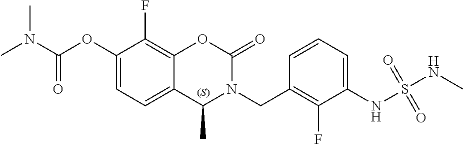 |
| | or |
| | 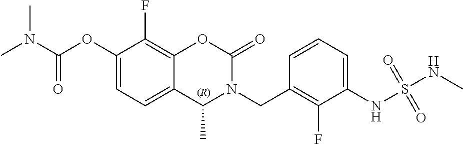 |
| 104 | 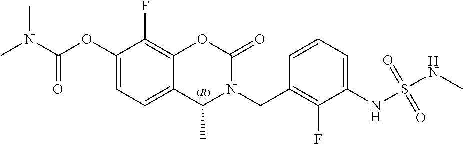 |
| | or |
| | 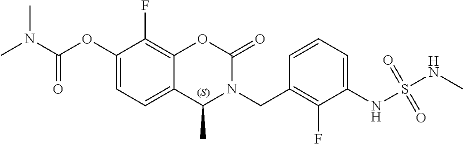 |
| 105 | 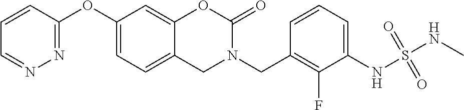 [SMILES: CNS(=O)(=O)Nc1cccc(CN2Cc3ccc(Oc4cccnn4)cc3OC2=O)c1F] |
| 106 | 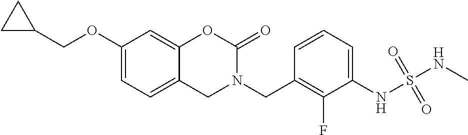 [SMILES: CNS(=O)(=O)Nc1cccc(CN2Cc3ccc(OCC4CC4)cc3OC2=O)c1F] |
| 107 | 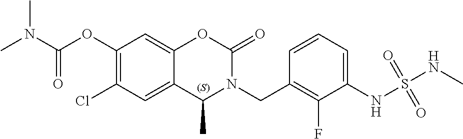 |
| | or |
| | 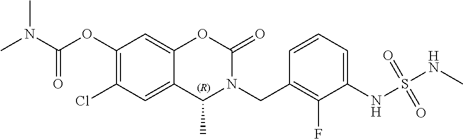 |
| 108 | 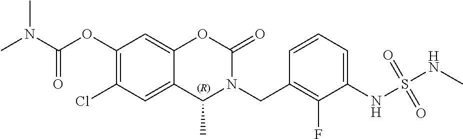 |
| | or |
| | 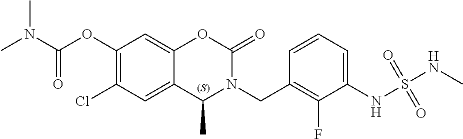 |
| 109 | 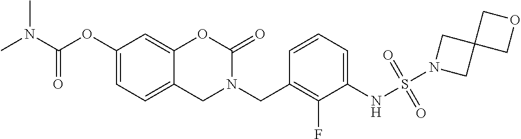 [SMILES: CN(C)C(=O)Oc1ccc2c(c1)OC(=O)N(Cc1cccc(NS(=O)(=O)N3CC4(COC4)C3)c1F)C2] |
| 110 | 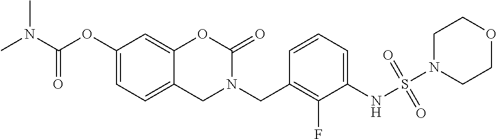 [SMILES: CN(C)C(=O)Oc1ccc2c(c1)OC(=O)N(Cc1cccc(NS(=O)(=O)N3CCOCC3)c1F)C2] |
| 111 | 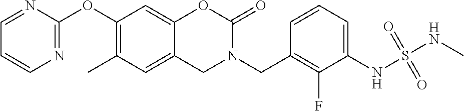 [SMILES: CNS(=O)(=O)Nc1cccc(CN2Cc3cc(C)c(Oc4ncccn4)cc3OC2=O)c1F] |
| 112 | 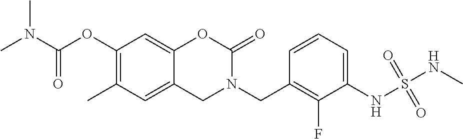 |
| 113 | 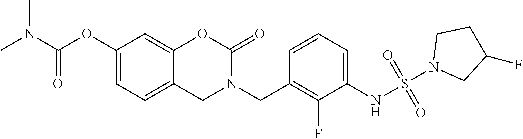 [SMILES: CN(C)C(=O)Oc1ccc2c(c1)OC(=O)N(Cc1cccc(NS(=O)(=O)N3CCC(F)C3)c1F)C2] |
| 114 | 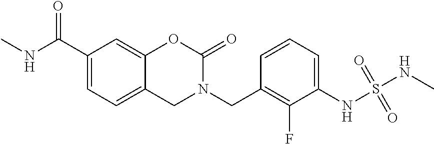 |
| 115 | 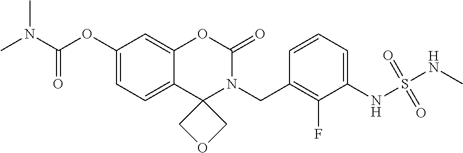 |
| 116 | 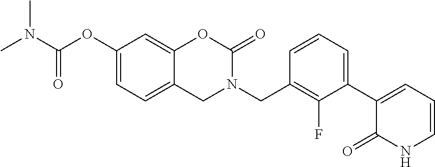 [SMILES: CN(C)C(=O)Oc1ccc2c(c1)OC(=O)N(Cc1cccc(-c3ccc[nH]c3=O)c1F)C2] |
| 117 | 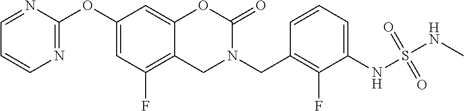 [SMILES: CNS(=O)(=O)Nc1cccc(CN2Cc3c(F)cc(Oc4ncccn4)cc3OC2=O)c1F] |
| 118 | 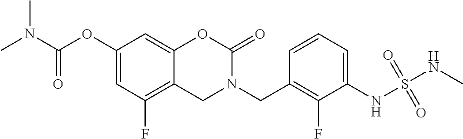 |
| 119 | 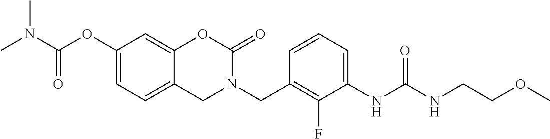 |
| 120 | 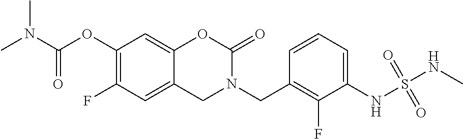 |
| 121 | 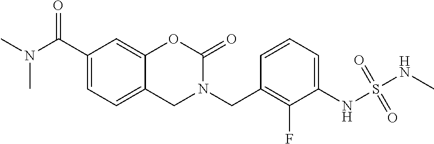 |
| 122 | 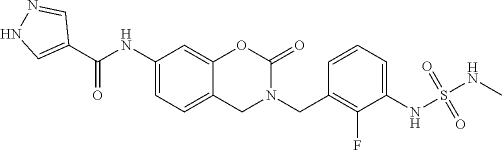 [SMILES: CNS(=O)(=O)Nc1cccc(CN2Cc3ccc(NC(=O)c4cn[nH]c4)cc3OC2=O)c1F] |
| 123 | 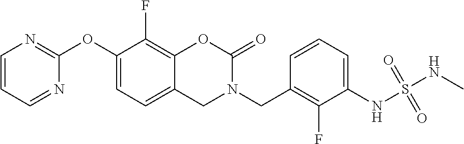 [SMILES: CNS(=O)(=O)Nc1cccc(CN2Cc3ccc(Oc4ncccn4)c(F)c3OC2=O)c1F] |
| 124 | 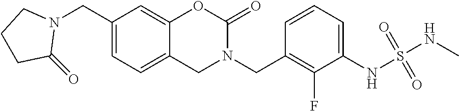 [SMILES: CNS(=O)(=O)Nc1cccc(CN2Cc3ccc(CN4CCCC4=O)cc3OC2=O)c1F] |
| 125 | 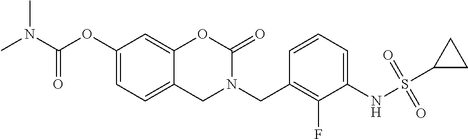 [SMILES: CN(C)C(=O)Oc1ccc2c(c1)OC(=O)N(Cc1cccc(NS(=O)(=O)C3CC3)c1F)C2] |
| 126 | 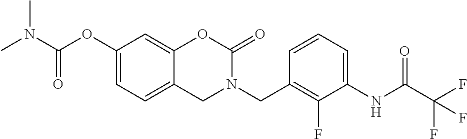 [SMILES: CN(C)C(=O)Oc1ccc2c(c1)OC(=O)N(Cc1cccc(NC(=O)C(F)(F)F)c1F)C2] |
| 127 | 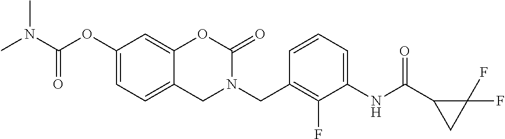 [SMILES: CN(C)C(=O)Oc1ccc2c(c1)OC(=O)N(Cc1cccc(NC(=O)C3CC3(F)F)c1F)C2] |
| 128 | 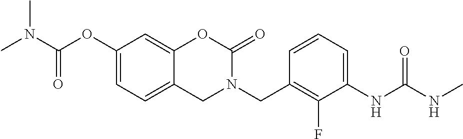 |
| 129 | 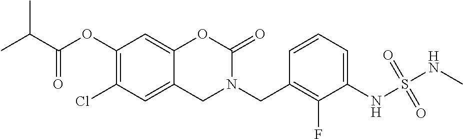 [SMILES: CNS(=O)(=O)Nc1cccc(CN2Cc3cc(Cl)c(OC(=O)C(C)C)cc3OC2=O)c1F] |
| 130 | 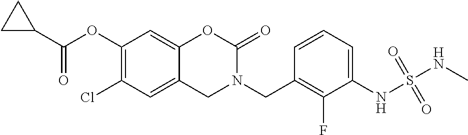 [SMILES: CNS(=O)(=O)Nc1cccc(CN2Cc3cc(Cl)c(OC(=O)C4CC4)cc3OC2=O)c1F] |
| 131 | 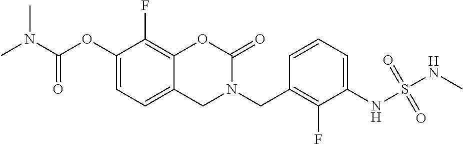 |
| 132 | 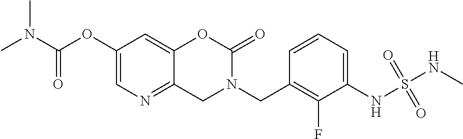 |
| 133 | 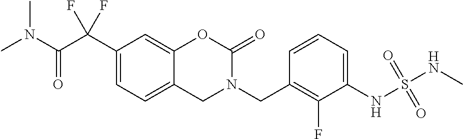 |
| 134 | 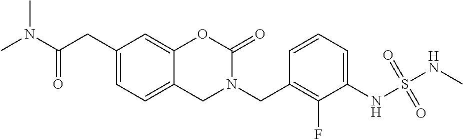 |
| 135 | 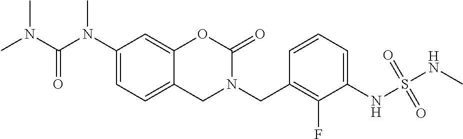 |
| 136 | 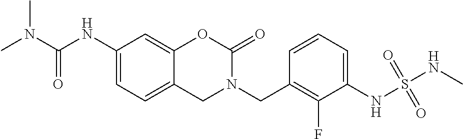 |
| 137 | 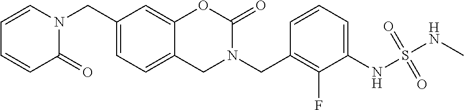 [SMILES: CNS(=O)(=O)Nc1cccc(CN2Cc3ccc(Cn4ccccc4=O)cc3OC2=O)c1F] |
| 138 | 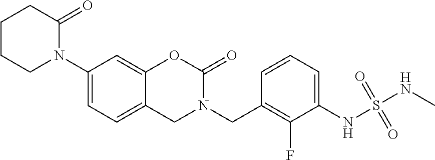 [SMILES: CNS(=O)(=O)Nc1cccc(CN2Cc3ccc(N4CCCCC4=O)cc3OC2=O)c1F] |
| 139 | 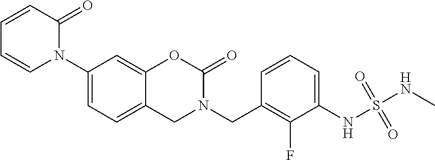 [SMILES: CNS(=O)(=O)Nc1cccc(CN2Cc3ccc(-n4ccccc4=O)cc3OC2=O)c1F] |
| 140 | 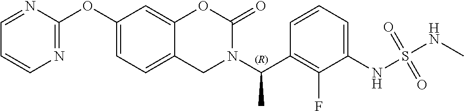 |
| | or |
| | 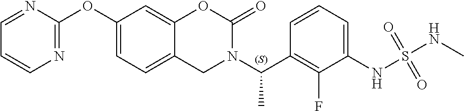 |
| 141 | 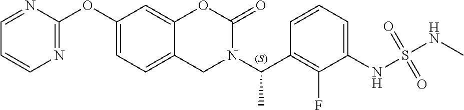 |
| | or |
| | 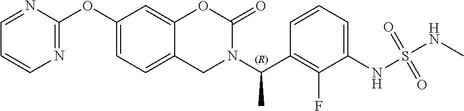 |
| 142 | 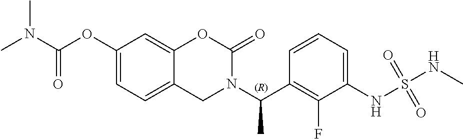 |
| | or |
| | 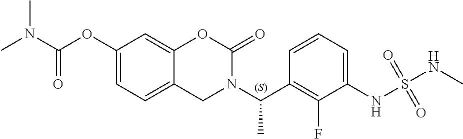 |
| 143 | 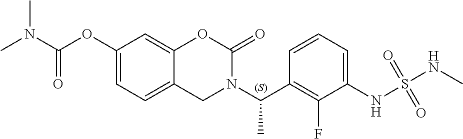 |
| | or |
| | 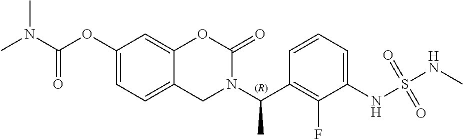 |
| 144 | 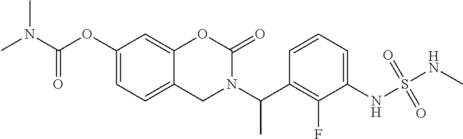 |
| 145 | 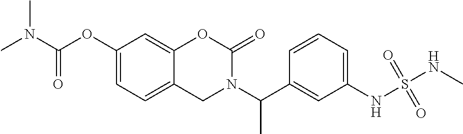 |
| 146 | 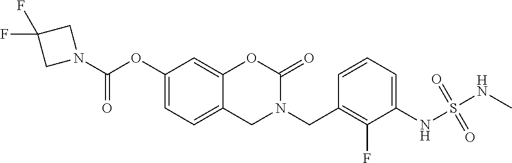 [SMILES: CNS(=O)(=O)Nc1cccc(CN2Cc3ccc(OC(=O)N4CC(F)(F)C4)cc3OC2=O)c1F] |
| 147 | 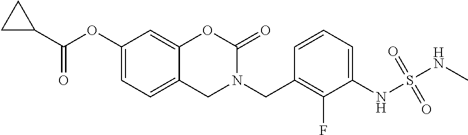 [SMILES: CNS(=O)(=O)Nc1cccc(CN2Cc3ccc(OC(=O)C4CC4)cc3OC2=O)c1F] |
| 148 | 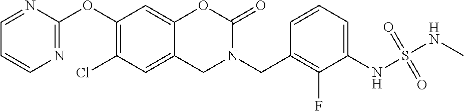 [SMILES: CNS(=O)(=O)Nc1cccc(CN2Cc3cc(Cl)c(Oc4ncccn4)cc3OC2=O)c1F] |
| 149 | 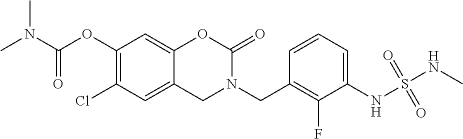 |
| 150 | 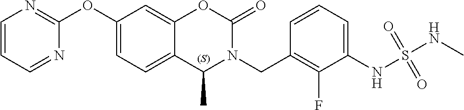 |
| | or |
| | 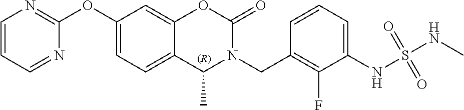 |
| 151 | 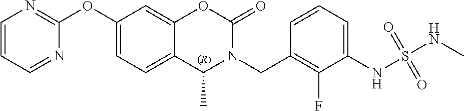 |
| | or |
| | 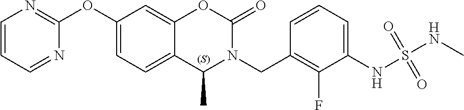 |
| 152 | 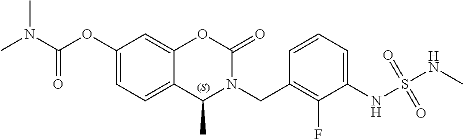 |
| | or |
| | 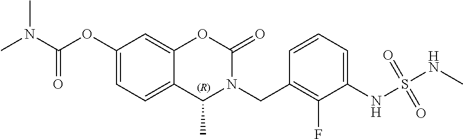 |
| 153 | 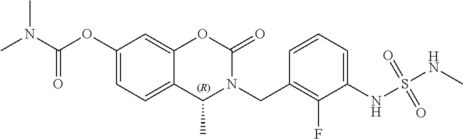 |
| | or |
| | 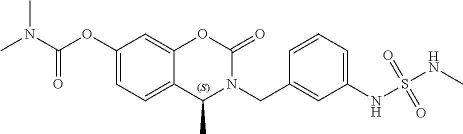 |
| 154 | 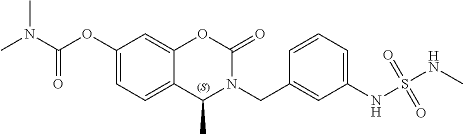 |
| | or |
| | 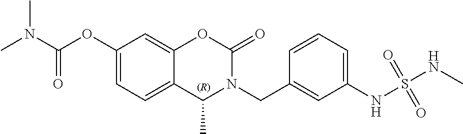 |
| 155 | 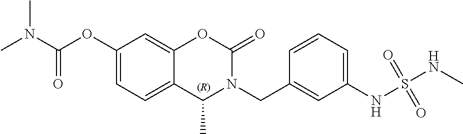 |
| | or |
| | 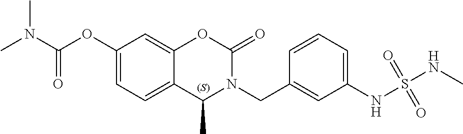 |
| 156 | 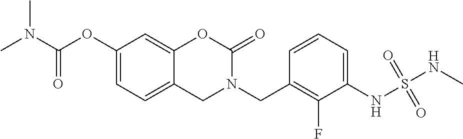 |
| 157 | 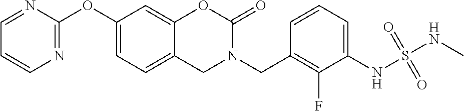 [SMILES: CNS(=O)(=O)Nc1cccc(CN2Cc3ccc(Oc4ncccn4)cc3OC2=O)c1F] |
| 158 | 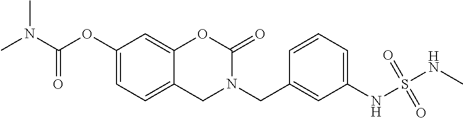 |
| 159 | 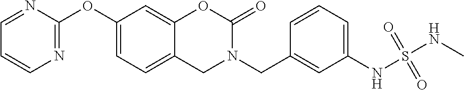 [SMILES: CNS(=O)(=O)Nc1cccc(CN2Cc3ccc(Oc4ncccn4)cc3OC2=O)c1] |
| 160 | 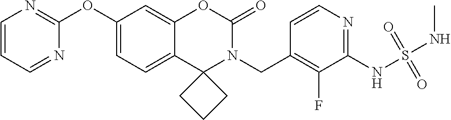 [SMILES: CNS(=O)(=O)Nc1nccc(CN2C(=O)Oc3cc(Oc4ncccn4)ccc3C23CCC3)c1F] |
| 161 | 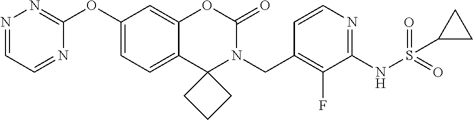 [SMILES: O=C1Oc2cc(Oc3nccnn3)ccc2C2(CCC2)N1Cc1ccnc(NS(=O)(=O)C2CC2)c1F] |
| 162 | 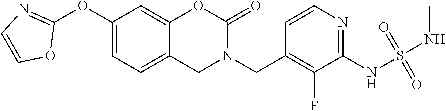 [SMILES: CNS(=O)(=O)Nc1nccc(CN2Cc3ccc(Oc4ncco4)cc3OC2=O)c1F] |
| 163 | 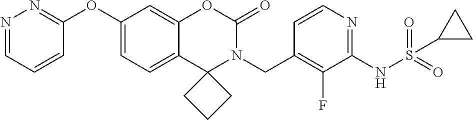 [SMILES: O=C1Oc2cc(Oc3cccnn3)ccc2C2(CCC2)N1Cc1ccnc(NS(=O)(=O)C2CC2)c1F] |
| 164 | 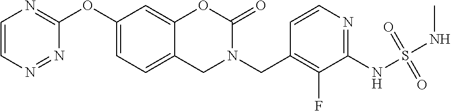 [SMILES: CNS(=O)(=O)Nc1nccc(CN2Cc3ccc(Oc4nccnn4)cc3OC2=O)c1F] |
| 165 | 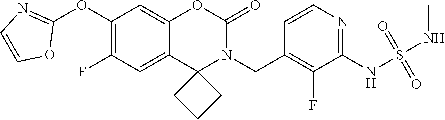 [SMILES: CNS(=O)(=O)Nc1nccc(CN2C(=O)Oc3cc(Oc4ncco4)c(F)cc3C23CCC3)c1F] |
| 166 | 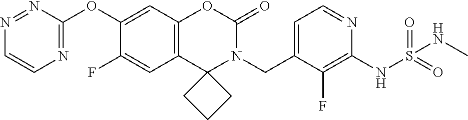 [SMILES: CNS(=O)(=O)Nc1nccc(CN2C(=O)Oc3cc(Oc4nccnn4)c(F)cc3C23CCC3)c1F] |
| 167 | 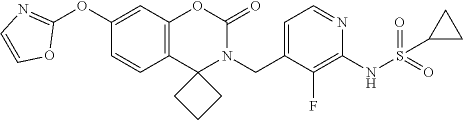 [SMILES: O=C1Oc2cc(Oc3ncco3)ccc2C2(CCC2)N1Cc1ccnc(NS(=O)(=O)C2CC2)c1F] |
| 168 | 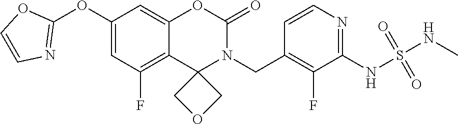 [SMILES: CNS(=O)(=O)Nc1nccc(CN2C(=O)Oc3cc(Oc4ncco4)cc(F)c3C23COC3)c1F] |
| 169 | 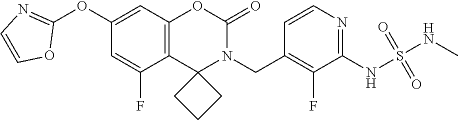 [SMILES: CNS(=O)(=O)Nc1nccc(CN2C(=O)Oc3cc(Oc4ncco4)cc(F)c3C23CCC3)c1F] |
| 170 | 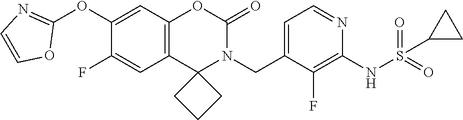 [SMILES: O=C1Oc2cc(Oc3ncco3)c(F)cc2C2(CCC2)N1Cc1ccnc(NS(=O)(=O)C2CC2)c1F] |
| 171 | 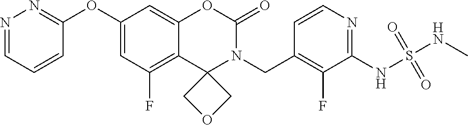 [SMILES: CNS(=O)(=O)Nc1nccc(CN2C(=O)Oc3cc(Oc4cccnn4)cc(F)c3C23COC3)c1F] |
| 172 | 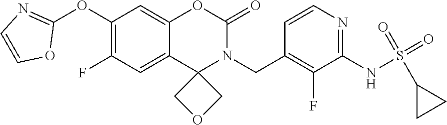 [SMILES: O=C1Oc2cc(Oc3ncco3)c(F)cc2C2(COC2)N1Cc1ccnc(NS(=O)(=O)C2CC2)c1F] |
| 173 | 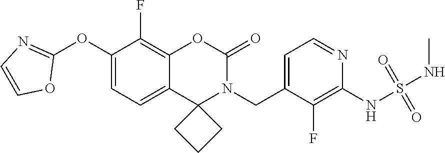 [SMILES: CNS(=O)(=O)Nc1nccc(CN2C(=O)Oc3c(ccc(Oc4ncco4)c3F)C23CCC3)c1F] |
| 174 | 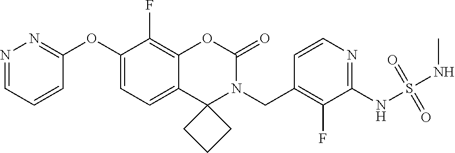 [SMILES: CNS(=O)(=O)Nc1nccc(CN2C(=O)Oc3c(ccc(Oc4cccnn4)c3F)C23CCC3)c1F] |
| 175 | 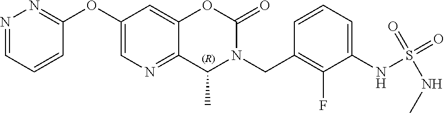 |
| | or |
| | 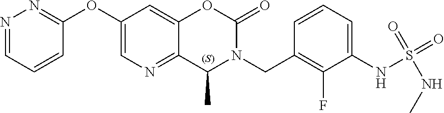 |
| 176 | 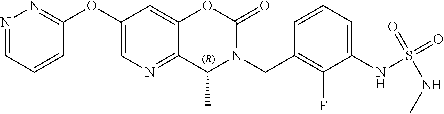 |
| | or |
| | 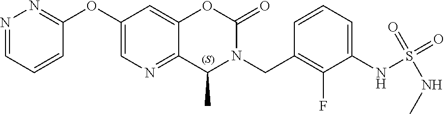 |
| 177 | 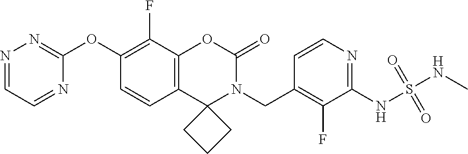 [SMILES: CNS(=O)(=O)Nc1nccc(CN2C(=O)Oc3c(ccc(Oc4nccnn4)c3F)C23CCC3)c1F] |
| 178 | 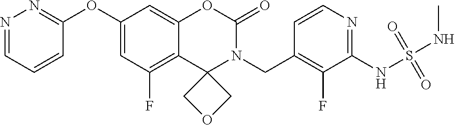 [SMILES: CNS(=O)(=O)Nc1nccc(CN2C(=O)Oc3cc(Oc4cccnn4)cc(F)c3C23COC3)c1F] |
| 179 | 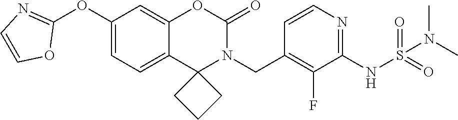 [SMILES: CN(C)S(=O)(=O)Nc1nccc(CN2C(=O)Oc3cc(Oc4ncco4)ccc3C23CCC3)c1F] |
| 180 | 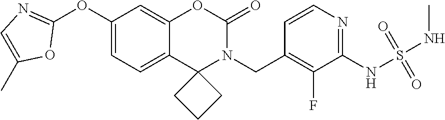 [SMILES: CNS(=O)(=O)Nc1nccc(CN2C(=O)Oc3cc(Oc4ncc(C)o4)ccc3C23CCC3)c1F] |
| 181 | 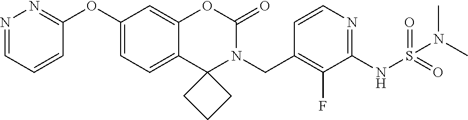 [SMILES: CN(C)S(=O)(=O)Nc1nccc(CN2C(=O)Oc3cc(Oc4cccnn4)ccc3C23CCC3)c1F] |
| 182 | 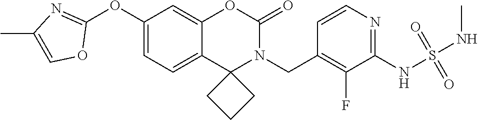 |
| 183 | 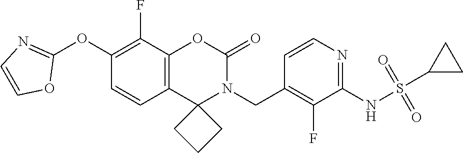 [SMILES: O=C1Oc2c(ccc(Oc3ncco3)c2F)C2(CCC2)N1Cc1ccnc(NS(=O)(=O)C2CC2)c1F] |
| 184 | 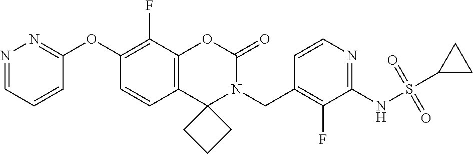 [SMILES: O=C1Oc2c(ccc(Oc3cccnn3)c2F)C2(CCC2)N1Cc1ccnc(NS(=O)(=O)C2CC2)c1F] |
| 185 | 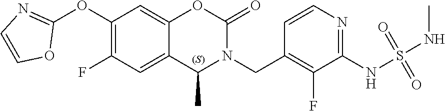 |
| | or |
| | 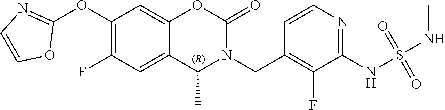 |
| 186 | 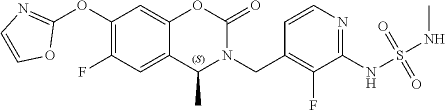 |
| | or |
| | 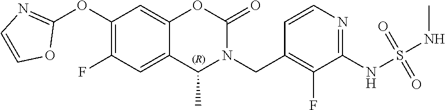 |
| 187 | 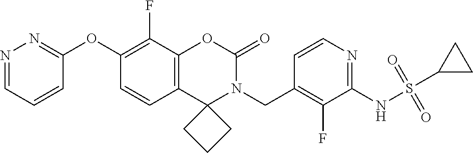 [SMILES: O=C1Oc2c(ccc(Oc3cccnn3)c2F)C2(CCC2)N1Cc1ccnc(NS(=O)(=O)C2CC2)c1F] |
| 188 | 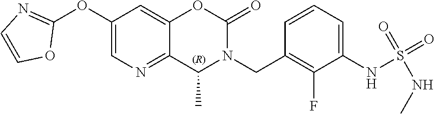 |
| | or |
| | 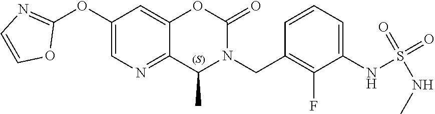 |
| 189 | 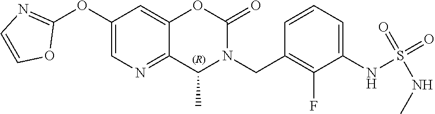 |
| | or |
| | 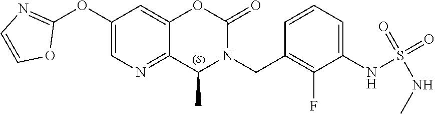 |
| 190 | 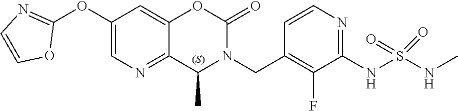 |
| | or |
| | 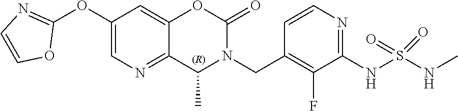 |
| 191 | 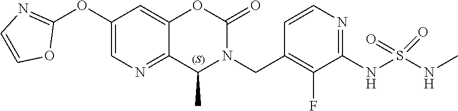 |
| | or |
| | 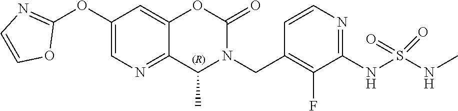 |
| 192 | 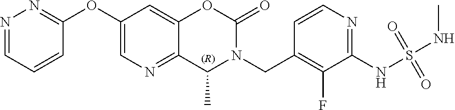 |
| | or |
| | 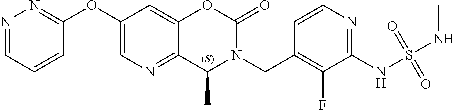 |
| 193 | 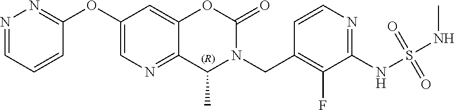 |
| | or |
| | 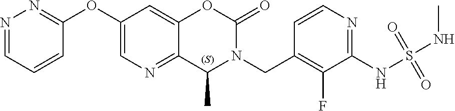 |
| 194 | 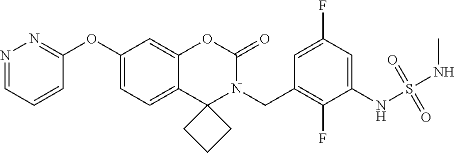 [SMILES: CNS(=O)(=O)Nc1cc(F)cc(CN2C(=O)Oc3cc(Oc4cccnn4)ccc3C23CCC3)c1F] |
| 195 | 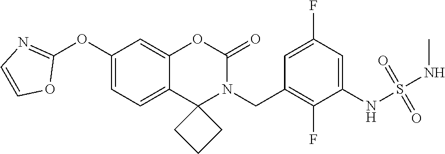 [SMILES: CNS(=O)(=O)Nc1cc(F)cc(CN2C(=O)Oc3cc(Oc4ncco4)ccc3C23CCC3)c1F] |
| 196 | 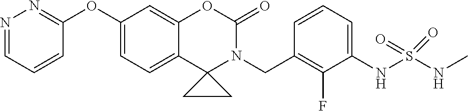 [SMILES: CNS(=O)(=O)Nc1cccc(CN2C(=O)Oc3cc(Oc4cccnn4)ccc3C23CC3)c1F] |
| 197 | 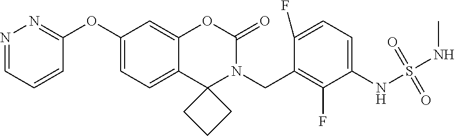 [SMILES: CNS(=O)(=O)Nc1ccc(F)c(CN2C(=O)Oc3cc(Oc4cccnn4)ccc3C23CCC3)c1F] |
| 198 | 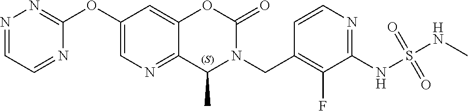 |
| | or |
| | 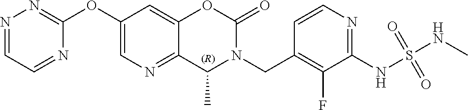 |
| 199 | 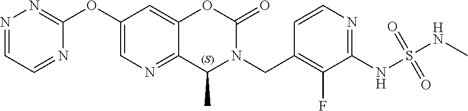 |
| | or |
| | 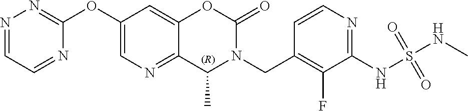 |
| 200 | 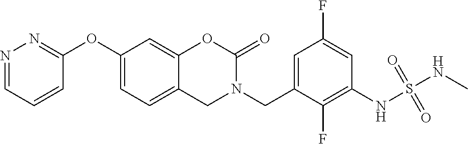 [SMILES: CNS(=O)(=O)Nc1cc(F)cc(CN2Cc3ccc(Oc4cccnn4)cc3OC2=O)c1F] |
| 201 | 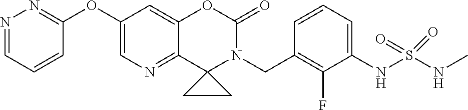 [SMILES: CNS(=O)(=O)Nc1cccc(CN2C(=O)Oc3cc(Oc4cccnn4)cnc3C23CC3)c1F] |
| 202 | 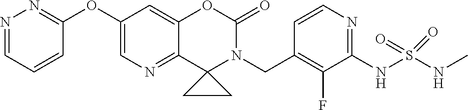 [SMILES: CNS(=O)(=O)Nc1nccc(CN2C(=O)Oc3cc(Oc4cccnn4)cnc3C23CC3)c1F] |

In certain embodiments, the compound is selected from the compound of Table 2 or a pharmaceutically acceptable salt thereof.

TABLE 2
List of exemplified compounds

| Compound | |
|---|---|
| No. | Structure |
| 1 | 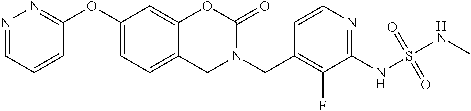 [SMILES: CNS(=O)(=O)Nc1nccc(CN2Cc3ccc(Oc4cccnn4)cc3OC2=O)c1F] |
| 2 | 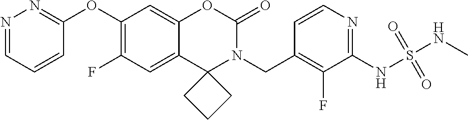 [SMILES: CNS(=O)(=O)Nc1nccc(CN2C(=O)Oc3cc(Oc4cccnn4)c(F)cc3C23CCC3)c1F] |
| 3 | 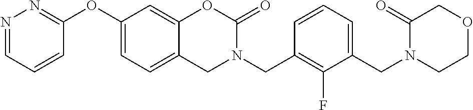 [SMILES: O=C1COCCN1Cc1cccc(CN2Cc3ccc(Oc4cccnn4)cc3OC2=O)c1F] |
| 4 | 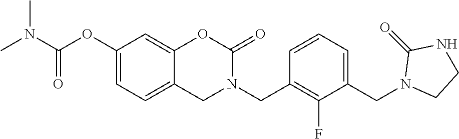 [SMILES: CN(C)C(=O)Oc1ccc2c(c1)OC(=O)N(Cc1cccc(CN3CCNC3=O)c1F)C2] |
| 5 | 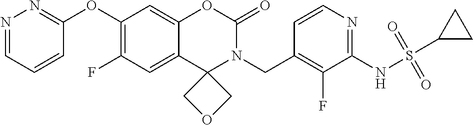 [SMILES: O=C1Oc2cc(Oc3cccnn3)c(F)cc2C2(COC2)N1Cc1ccnc(NS(=O)(=O)C2CC2)c1F] |
| 6 | 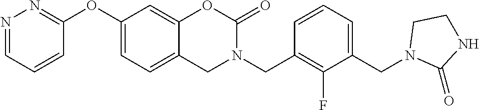 [SMILES: O=C1NCCN1Cc1cccc(CN2Cc3ccc(Oc4cccnn4)cc3OC2=O)c1F] |
| 7 | 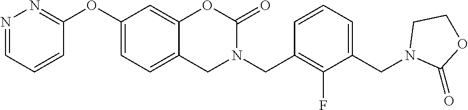 [SMILES: O=C1OCCN1Cc1cccc(CN2Cc3ccc(Oc4cccnn4)cc3OC2=O)c1F] |
| 8 | 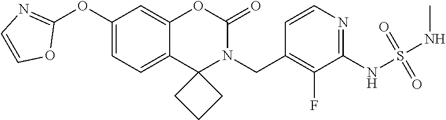 [SMILES: CNS(=O)(=O)Nc1nccc(CN2C(=O)Oc3cc(Oc4ncco4)ccc3C23CCC3)c1F] |
| 9 | 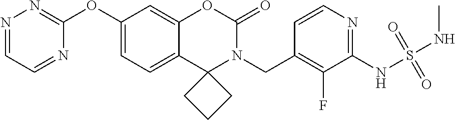 [SMILES: CNS(=O)(=O)Nc1nccc(CN2C(=O)Oc3cc(Oc4nccnn4)ccc3C23CCC3)c1F] |
| 10 | 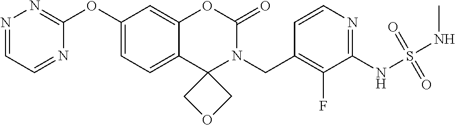 [SMILES: CNS(=O)(=O)Nc1nccc(CN2C(=O)Oc3cc(Oc4nccnn4)ccc3C23COC3)c1F] |
| 11 | 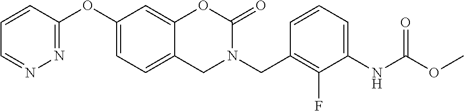 [SMILES: COC(=O)Nc1cccc(CN2Cc3ccc(Oc4cccnn4)cc3OC2=O)c1F] |
| 12 | 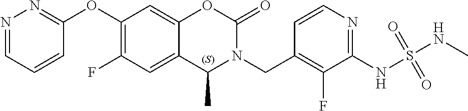 |
| | or |
| | 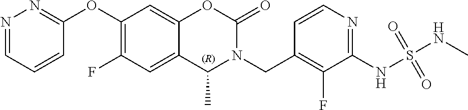 |
| 13 | 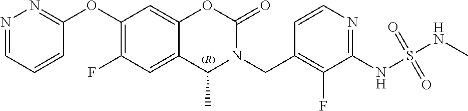 |
| | or |
| | 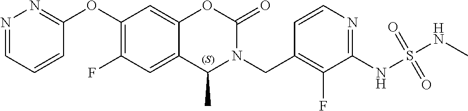 |
| 14 | 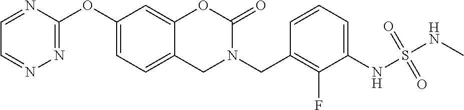 [SMILES: CNS(=O)(=O)Nc1cccc(CN2Cc3ccc(Oc4nccnn4)cc3OC2=O)c1F] |
| 15 | 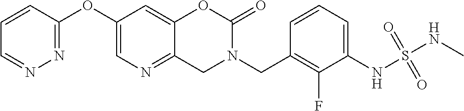 [SMILES: CNS(=O)(=O)Nc1cccc(CN2Cc3ncc(Oc4cccnn4)cc3OC2=O)c1F] |
| 17 | 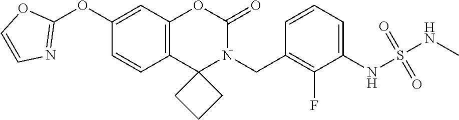 [SMILES: CNS(=O)(=O)Nc1cccc(CN2C(=O)Oc3cc(Oc4ncco4)ccc3C23CCC3)c1F] |
| 18 | 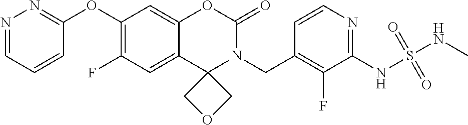 [SMILES: CNS(=O)(=O)Nc1nccc(CN2C(=O)Oc3cc(Oc4cccnn4)c(F)cc3C23COC3)c1F] |
| 19 | 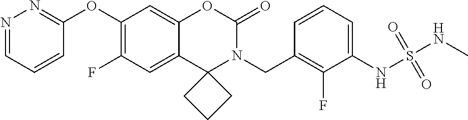 [SMILES: CNS(=O)(=O)Nc1cccc(CN2C(=O)Oc3cc(Oc4cccnn4)c(F)cc3C23CCC3)c1F] |
| 20 | 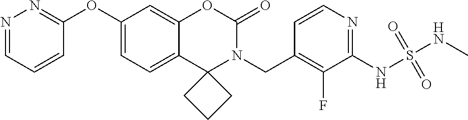 [SMILES: CNS(=O)(=O)Nc1nccc(CN2C(=O)Oc3cc(Oc4cccnn4)ccc3C23CCC3)c1F] |
| 21 | 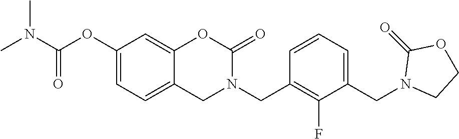 [SMILES: CN(C)C(=O)Oc1ccc2c(c1)OC(=O)N(Cc1cccc(CN3CCOC3=O)c1F)C2] |
| 22 | 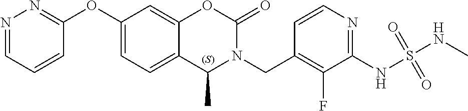 |
| | or |
| | 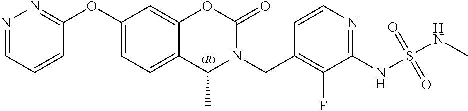 |
| 23 | 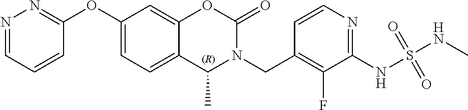 |
| | or |
| | 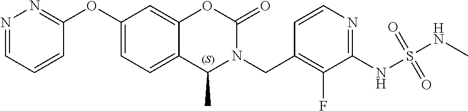 |
| 24 | 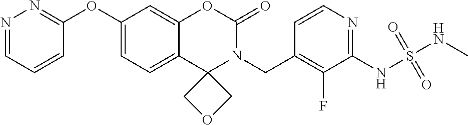 [SMILES: CNS(=O)(=O)Nc1nccc(CN2C(=O)Oc3cc(Oc4cccnn4)ccc3C23COC3)c1F] |
| 25 | 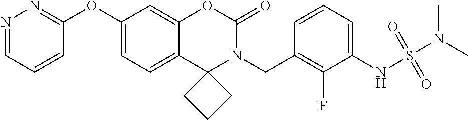 [SMILES: CN(C)S(=O)(=O)Nc1cccc(CN2C(=O)Oc3cc(Oc4cccnn4)ccc3C23CCC3)c1F] |
| 26 | 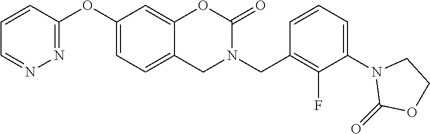 [SMILES: O=C1Oc2cc(Oc3cccnn3)ccc2CN1Cc1cccc(N2CCOC2=O)c1F] |
| 27 | 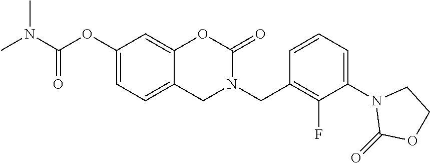 [SMILES: CN(C)C(=O)Oc1ccc2c(c1)OC(=O)N(Cc1cccc(N3CCOC3=O)c1F)C2] |
| 31 | 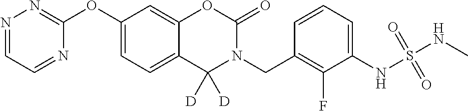 [SMILES: [2H]C1([2H])c2ccc(Oc3nccnn3)cc2OC(=O)N1Cc1cccc(NS(=O)(=O)NC)c1F] |
| 32 | 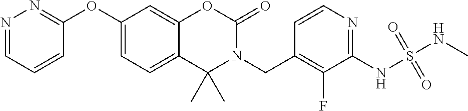 [SMILES: CNS(=O)(=O)Nc1nccc(CN2C(=O)Oc3cc(Oc4cccnn4)ccc3C2(C)C)c1F] |
| 33 | 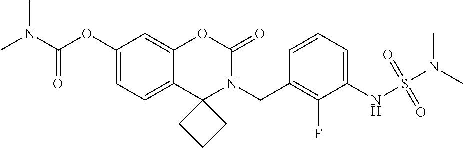 |
| 34 | 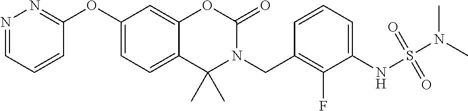 [SMILES: CN(C)S(=O)(=O)Nc1cccc(CN2C(=O)Oc3cc(Oc4cccnn4)ccc3C2(C)C)c1F] |
| 35 | 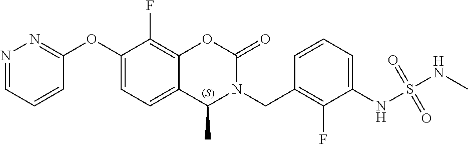 |
| | or |
| | 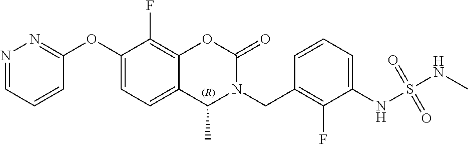 |
| 36 | 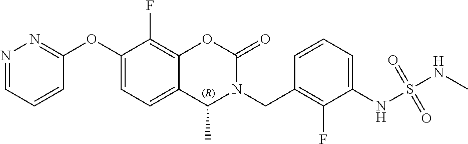 |
| | or |
| | 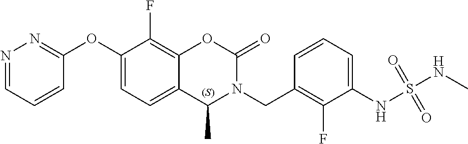 |
| 37 | 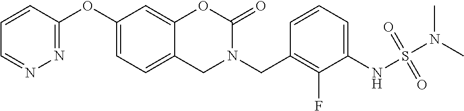 [SMILES: CN(C)S(=O)(=O)Nc1cccc(CN2Cc3ccc(Oc4cccnn4)cc3OC2=O)c1F] |
| 38 | 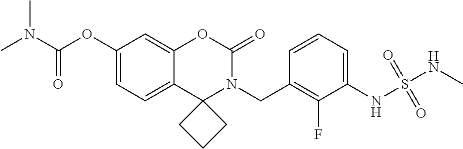 |
| 39 | 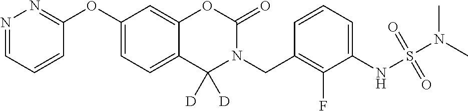 [SMILES: [2H]C1([2H])c2ccc(Oc3cccnn3)cc2OC(=O)N1Cc1cccc(NS(=O)(=O)N(C)C)c1F] |
| 40 | 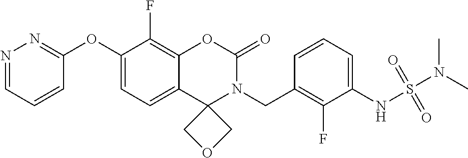 [SMILES: CN(C)S(=O)(=O)Nc1cccc(CN2C(=O)Oc3c(ccc(Oc4cccnn4)c3F)C23COC3)c1F] |
| 41 | 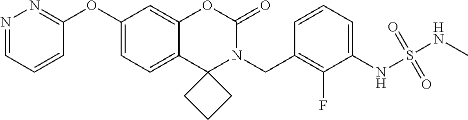 [SMILES: CNS(=O)(=O)Nc1cccc(CN2C(=O)Oc3cc(Oc4cccnn4)ccc3C23CCC3)c1F] |
| 42 | 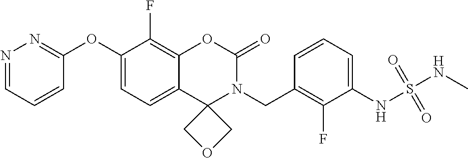 [SMILES: CNS(=O)(=O)Nc1cccc(CN2C(=O)Oc3c(ccc(Oc4cccnn4)c3F)C23COC3)c1F] |
| 43 | 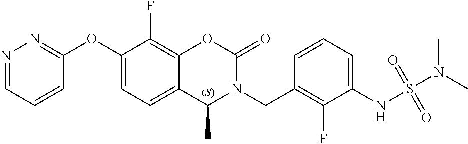 |
| | or |
| | 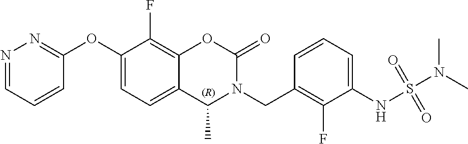 |
| 44 | 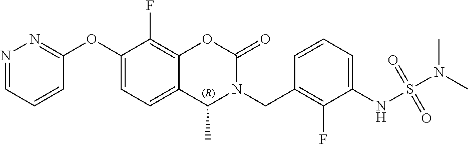 |
| | or |
| | 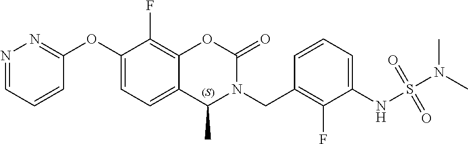 |
| 45 | 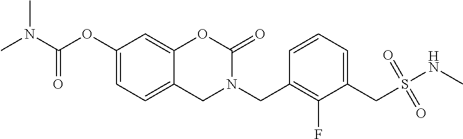 |
| 46 | 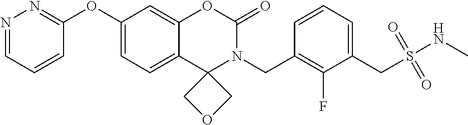 [SMILES: CNS(=O)(=O)Cc1cccc(CN2C(=O)Oc3cc(Oc4cccnn4)ccc3C23COC3)c1F] |
| 47 | 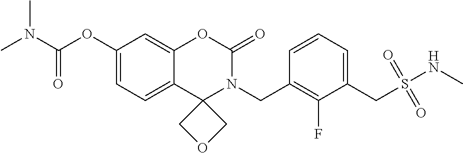 |
| 48 | 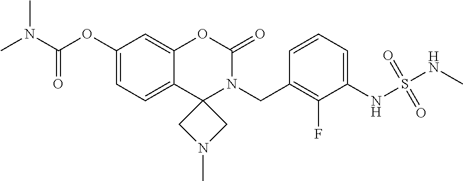 |
| 49 | 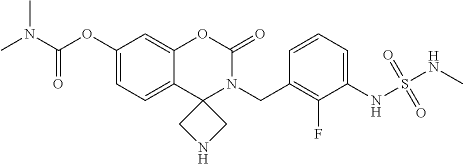 |
| 50 | 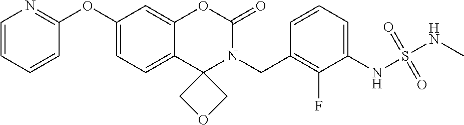 [SMILES: CNS(=O)(=O)Nc1cccc(CN2C(=O)Oc3cc(Oc4ccccn4)ccc3C23COC3)c1F] |
| 51 | 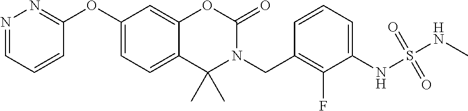 [SMILES: CNS(=O)(=O)Nc1cccc(CN2C(=O)Oc3cc(Oc4cccnn4)ccc3C2(C)C)c1F] |
| 52 | 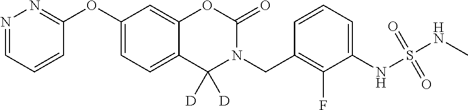 [SMILES: [2H]C1([2H])c2ccc(Oc3cccnn3)cc2OC(=O)N1Cc1cccc(NS(=O)(=O)NC)c1F] |
| 53 | 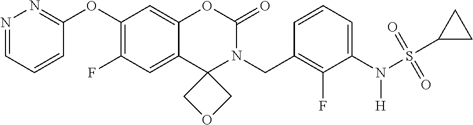 [SMILES: O=C1Oc2cc(Oc3cccnn3)c(F)cc2C2(COC2)N1Cc1cccc(NS(=O)(=O)C2CC2)c1F] |
| 54 | 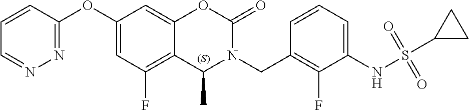 |
| | or |
| | 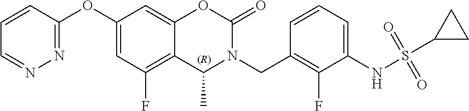 |
| 55 | 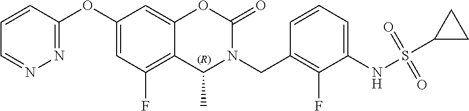 |
| | or |
| | 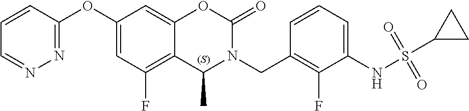 |
| 56 | 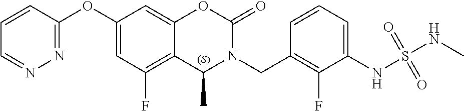 |
| | or |
| | 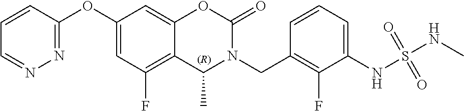 |
| 57 | 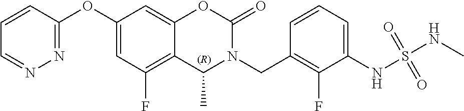 |
| | or |
| | 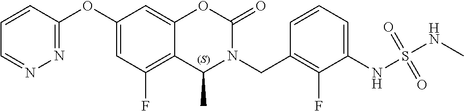 |
| 58 | 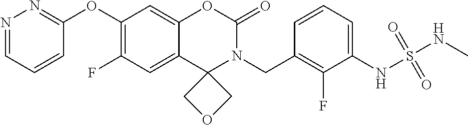 [SMILES: CNS(=O)(=O)Nc1cccc(CN2C(=O)Oc3cc(Oc4cccnn4)c(F)cc3C23COC3)c1F] |
| 59 | 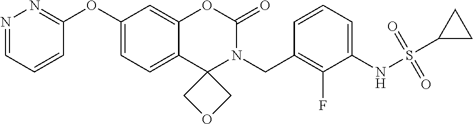 [SMILES: O=C1Oc2cc(Oc3cccnn3)ccc2C2(COC2)N1Cc1cccc(NS(=O)(=O)C2CC2)c1F] |
| 63 | 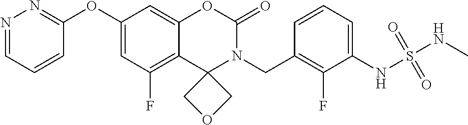 [SMILES: CNS(=O)(=O)Nc1cccc(CN2C(=O)Oc3cc(Oc4cccnn4)cc(F)c3C23COC3)c1F] |
| 64 | 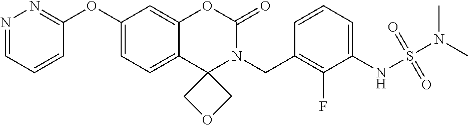 [SMILES: CN(C)S(=O)(=O)Nc1cccc(CN2C(=O)Oc3cc(Oc4cccnn4)ccc3C23COC3)c1F] |
| 66 | 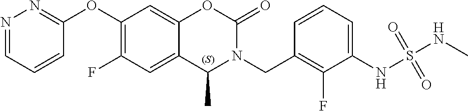 |
| | or |
| | 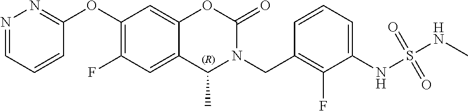 |
| 67 | 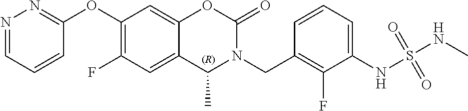 |
| | or |
| | 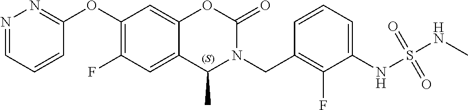 |
| 68 | 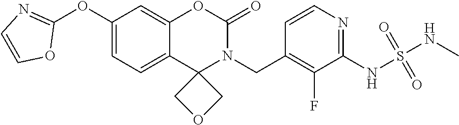 [SMILES: CNS(=O)(=O)Nc1nccc(CN2C(=O)Oc3cc(Oc4ncco4)ccc3C23COC3)c1F] |
| 69 | 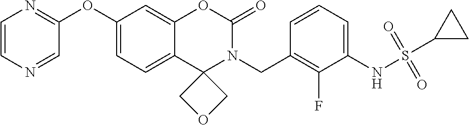 [SMILES: O=C1Oc2cc(Oc3cnccn3)ccc2C2(COC2)N1Cc1cccc(NS(=O)(=O)C2CC2)c1F] |
| 70 | 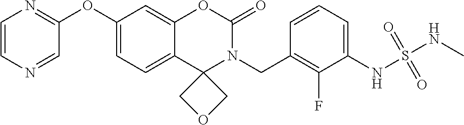 [SMILES: CNS(=O)(=O)Nc1cccc(CN2C(=O)Oc3cc(Oc4cnccn4)ccc3C23COC3)c1F] |
| 71 | 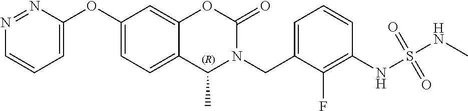 |
| | or |
| | 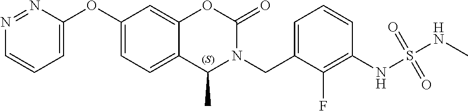 |
| 72 | 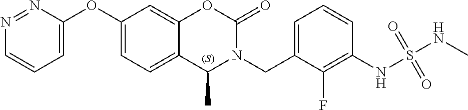 |
| | or |
| | 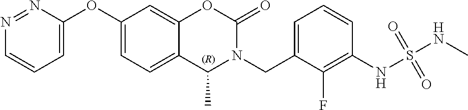 |
| 73 | 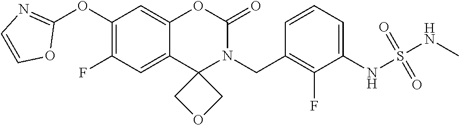 [SMILES: CNS(=O)(=O)Nc1cccc(CN2C(=O)Oc3cc(Oc4ncco4)c(F)cc3C23COC3)c1F] |
| 74 | 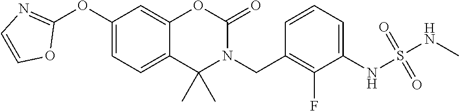 [SMILES: CNS(=O)(=O)Nc1cccc(CN2C(=O)Oc3cc(Oc4ncco4)ccc3C2(C)C)c1F] |
| 75 | 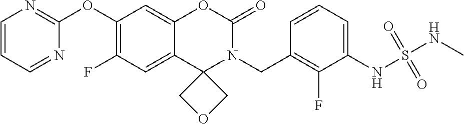 [SMILES: CNS(=O)(=O)Nc1cccc(CN2C(=O)Oc3cc(Oc4ncccn4)c(F)cc3C23COC3)c1F] |
| 76 | 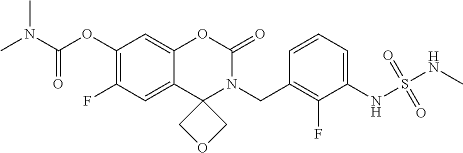 |
| 77 | 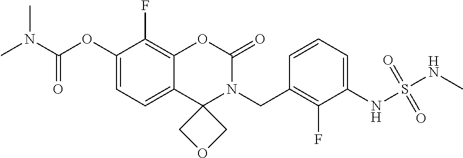 |
| 78 | 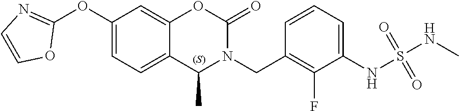 |
| | or |
| | 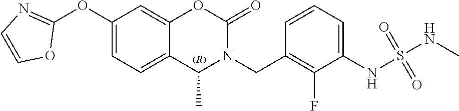 |
| 79 | 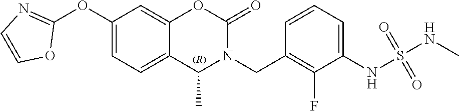 |
| | or |
| | 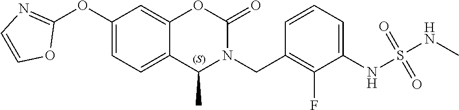 |
| 80 | 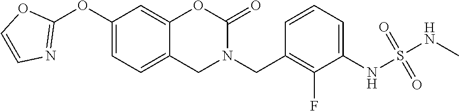 [SMILES: CNS(=O)(=O)Nc1cccc(CN2Cc3ccc(Oc4ncco4)cc3OC2=O)c1F] |
| 81 | 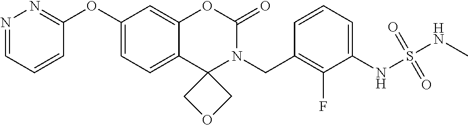 [SMILES: CNS(=O)(=O)Nc1cccc(CN2C(=O)Oc3cc(Oc4cccnn4)ccc3C23COC3)c1F] |
| 82 | 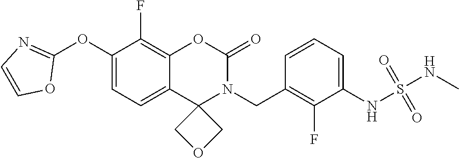 [SMILES: CNS(=O)(=O)Nc1cccc(CN2C(=O)Oc3c(ccc(Oc4ncco4)c3F)C23COC3)c1F] |
| 84 | 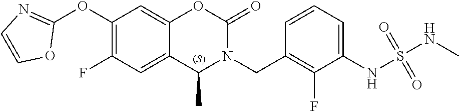 |
| | or |
| | 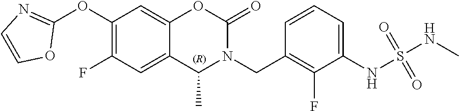 |
| 85 | 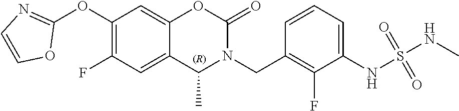 |
| | or |
| | 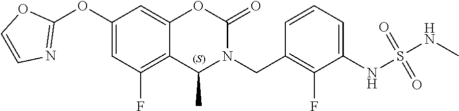 |
| 86 | 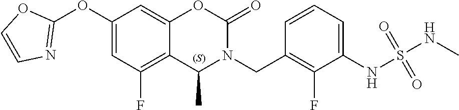 |
| | or |
| | 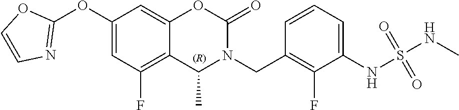 |
| 87 | 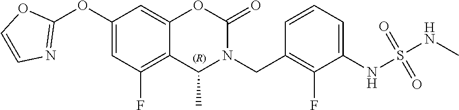 |
| | or |
| | 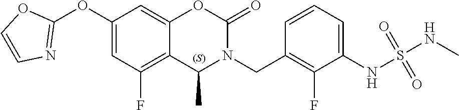 |
| 88 | 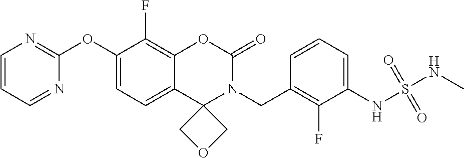 [SMILES: CNS(=O)(=O)Nc1cccc(CN2C(=O)Oc3c(ccc(Oc4ncccn4)c3F)C23COC3)c1F] |
| 89 | 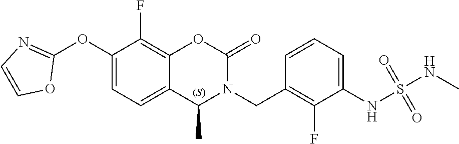 |
| | or |
| | 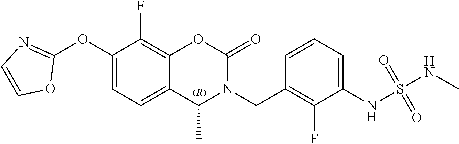 |
| 90 | 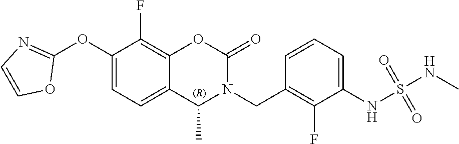 |
| | or |
| | 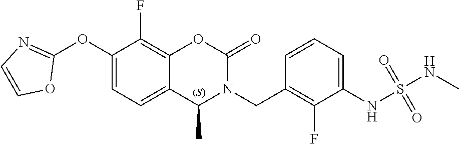 |
| 91 | 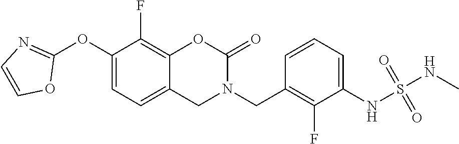 [SMILES: CNS(=O)(=O)Nc1cccc(CN2Cc3ccc(Oc4ncco4)c(F)c3OC2=O)c1F] |
| 94 | 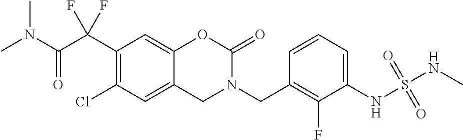 |
| 95 | 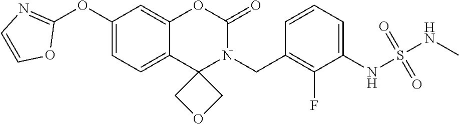 [SMILES: CNS(=O)(=O)Nc1cccc(CN2C(=O)Oc3cc(Oc4ncco4)ccc3C23COC3)c1F] |
| 98 | 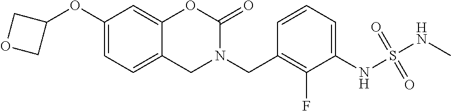 [SMILES: CNS(=O)(=O)Nc1cccc(CN2Cc3ccc(OC4COC4)cc3OC2=O)c1F] |
| 99 | 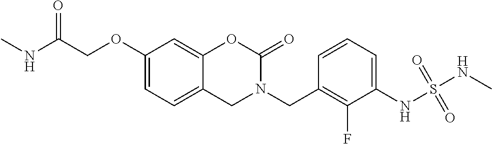 |
| 101 | 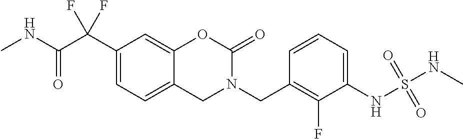 |
| 104 | 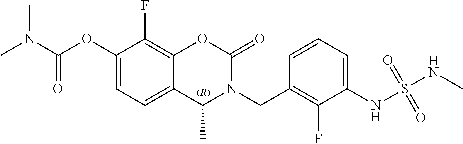 |
| | or |
| | 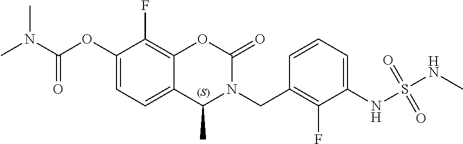 |
| 105 | 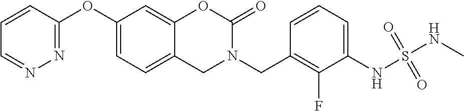 [SMILES: CNS(=O)(=O)Nc1cccc(CN2Cc3ccc(Oc4cccnn4)cc3OC2=O)c1F] |
| 106 | 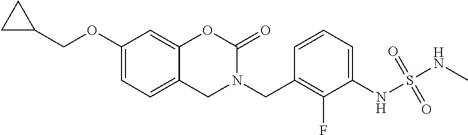 [SMILES: CNS(=O)(=O)Nc1cccc(CN2Cc3ccc(OCC4CC4)cc3OC2=O)c1F] |
| 114 | 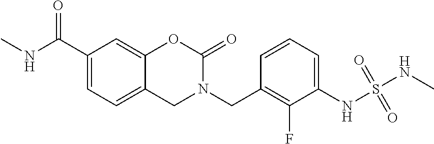 |
| 115 | 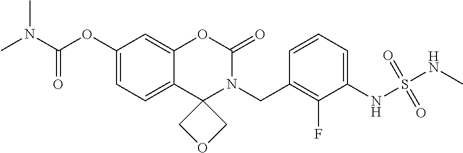 |
| 116 | 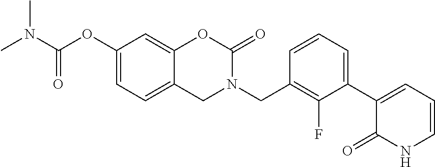 [SMILES: CN(C)C(=O)Oc1ccc2c(c1)OC(=O)N(Cc1cccc(-c3ccc[nH]c3=O)c1F)C2] |
| 119 | 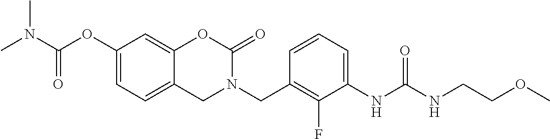 |
| 121 | 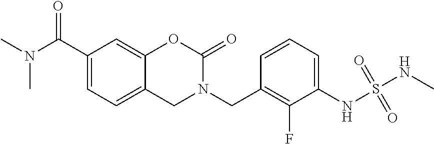 |
| 122 | 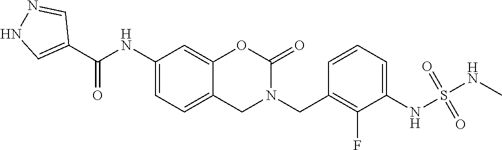 [SMILES: CNS(=O)(=O)Nc1cccc(CN2Cc3ccc(NC(=O)c4cn[nH]c4)cc3OC2=O)c1F] |
| 124 | 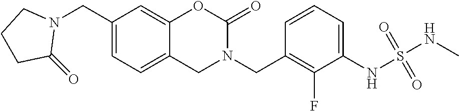 [SMILES: CNS(=O)(=O)Nc1cccc(CN2Cc3ccc(CN4CCCC4=O)cc3OC2=O)c1F] |
| 126 | 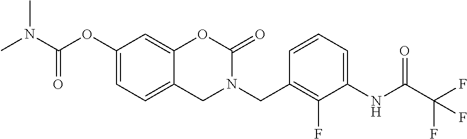 [SMILES: CN(C)C(=O)Oc1ccc2c(c1)OC(=O)N(Cc1cccc(NC(=O)C(F)(F)F)c1F)C2] |
| 127 | 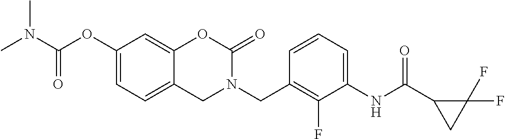 [SMILES: CN(C)C(=O)Oc1ccc2c(c1)OC(=O)N(Cc1cccc(NC(=O)C3CC3(F)F)c1F)C2] |
| 128 | 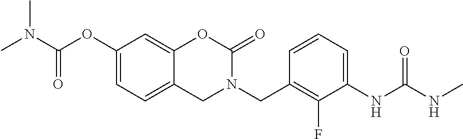 |
| 129 | 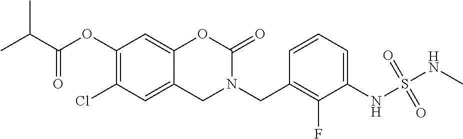 [SMILES: CNS(=O)(=O)Nc1cccc(CN2Cc3cc(Cl)c(OC(=O)C(C)C)cc3OC2=O)c1F] |
| 130 | 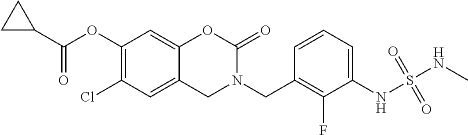 [SMILES: CNS(=O)(=O)Nc1cccc(CN2Cc3cc(Cl)c(OC(=O)C4CC4)cc3OC2=O)c1F] |
| 133 | 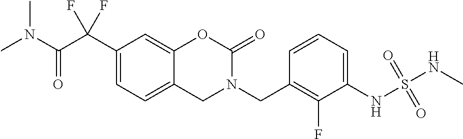 |
| 134 | 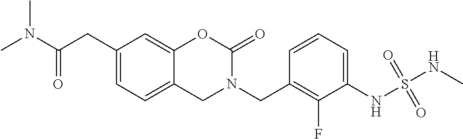 |
| 135 | 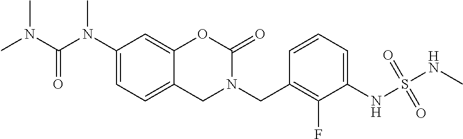 |
| 136 | 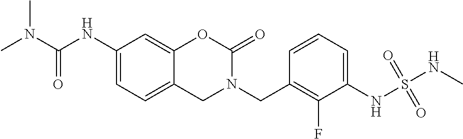 |
| 137 | 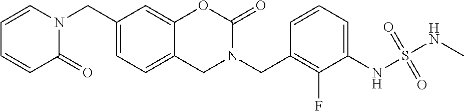 [SMILES: CNS(=O)(=O)Nc1cccc(CN2Cc3ccc(Cn4ccccc4=O)cc3OC2=O)c1F] |
| 138 | 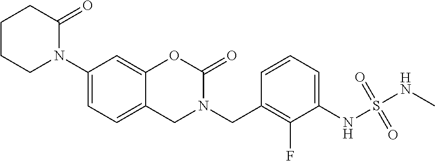 [SMILES: CNS(=O)(=O)Nc1cccc(CN2Cc3ccc(N4CCCCC4=O)cc3OC2=O)c1F] |
| 139 | 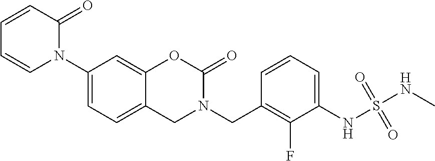 [SMILES: CNS(=O)(=O)Nc1cccc(CN2Cc3ccc(-n4ccccc4=O)cc3OC2=O)c1F] |
| 147 | 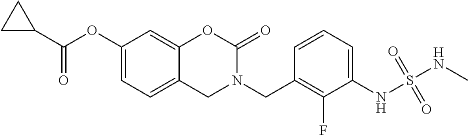 [SMILES: CNS(=O)(=O)Nc1cccc(CN2Cc3ccc(OC(=O)C4CC4)cc3OC2=O)c1F] |
| 160 | 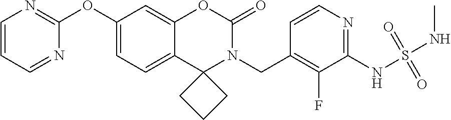 [SMILES: CNS(=O)(=O)Nc1nccc(CN2C(=O)Oc3cc(Oc4ncccn4)ccc3C23CCC3)c1F] |
| 161 | 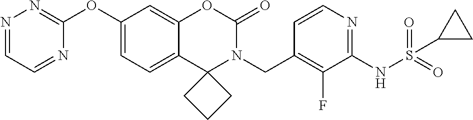 [SMILES: O=C1Oc2cc(Oc3nccnn3)ccc2C2(CCC2)N1Cc1ccnc(NS(=O)(=O)C2CC2)c1F] |
| 162 | 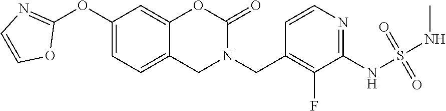 [SMILES: CNS(=O)(=O)Nc1nccc(CN2Cc3ccc(Oc4ncco4)cc3OC2=O)c1F] |
| 163 | 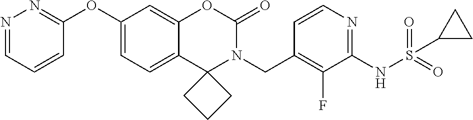 [SMILES: O=C1Oc2cc(Oc3cccnn3)ccc2C2(CCC2)N1Cc1ccnc(NS(=O)(=O)C2CC2)c1F] |
| 164 | 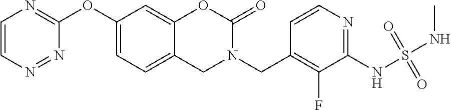 [SMILES: CNS(=O)(=O)Nc1nccc(CN2Cc3ccc(Oc4nccnn4)cc3OC2=O)c1F] |
| 165 | 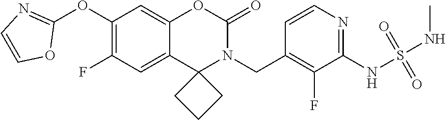 [SMILES: CNS(=O)(=O)Nc1nccc(CN2C(=O)Oc3cc(Oc4ncco4)c(F)cc3C23CCC3)c1F] |
| 166 | 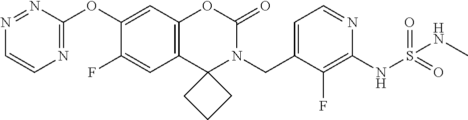 [SMILES: CNS(=O)(=O)Nc1nccc(CN2C(=O)Oc3cc(Oc4nccnn4)c(F)cc3C23CCC3)c1F] |
| 167 | 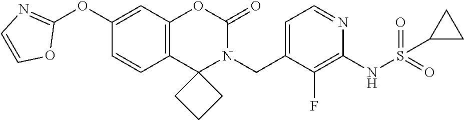 [SMILES: O=C1Oc2cc(Oc3ncco3)ccc2C2(CCC2)N1Cc1ccnc(NS(=O)(=O)C2CC2)c1F] |
| 168 | 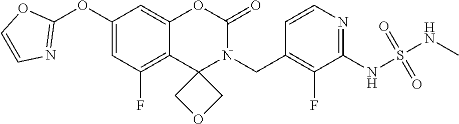 [SMILES: CNS(=O)(=O)Nc1nccc(CN2C(=O)Oc3cc(Oc4ncco4)cc(F)c3C23COC3)c1F] |
| 169 | 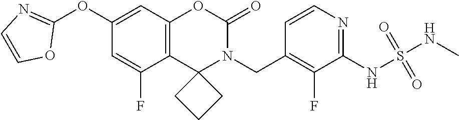 [SMILES: CNS(=O)(=O)Nc1nccc(CN2C(=O)Oc3cc(Oc4ncco4)cc(F)c3C23CCC3)c1F] |
| 170 | 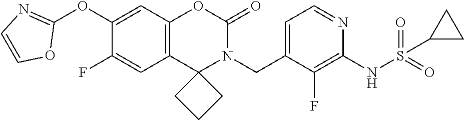 [SMILES: O=C1Oc2cc(Oc3ncco3)c(F)cc2C2(CCC2)N1Cc1ccnc(NS(=O)(=O)C2CC2)c1F] |
| 171 | 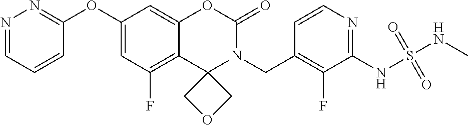 [SMILES: CNS(=O)(=O)Nc1nccc(CN2C(=O)Oc3cc(Oc4cccnn4)cc(F)c3C23COC3)c1F] |
| 172 | 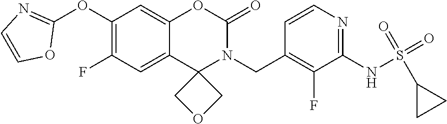 [SMILES: O=C1Oc2cc(Oc3ncco3)c(F)cc2C2(COC2)N1Cc1ccnc(NS(=O)(=O)C2CC2)c1F] |
| 173 | 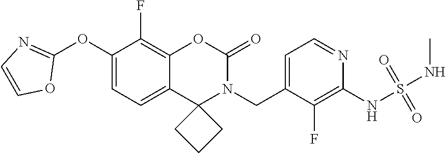 [SMILES: CNS(=O)(=O)Nc1nccc(CN2C(=O)Oc3c(ccc(Oc4ncco4)c3F)C23CCC3)c1F] |
| 174 | 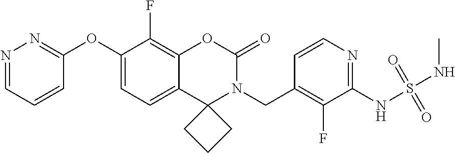 [SMILES: CNS(=O)(=O)Nc1nccc(CN2C(=O)Oc3c(ccc(Oc4cccnn4)c3F)C23CCC3)c1F] |
| 175 | 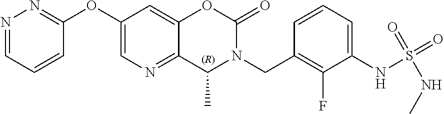 |
| | or |
| | 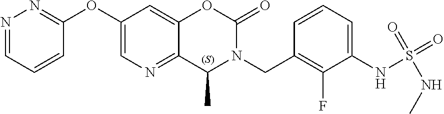 |
| 176 | 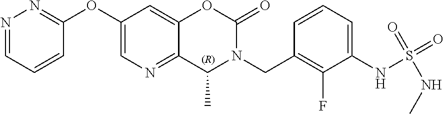 |
| | or |
| | 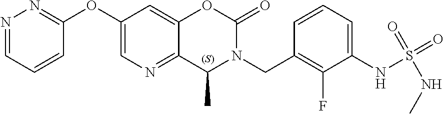 |
| 177 | 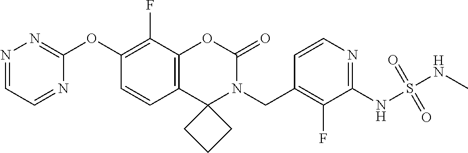 [SMILES: CNS(=O)(=O)Nc1nccc(CN2C(=O)Oc3c(ccc(Oc4nccnn4)c3F)C23CCC3)c1F] |
| 178 | 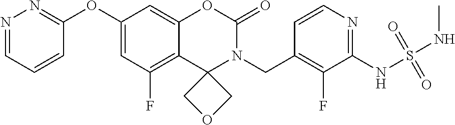 [SMILES: CNS(=O)(=O)Nc1nccc(CN2C(=O)Oc3cc(Oc4cccnn4)cc(F)c3C23COC3)c1F] |
| 179 | 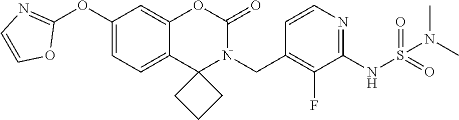 [SMILES: CN(C)S(=O)(=O)Nc1nccc(CN2C(=O)Oc3cc(Oc4ncco4)ccc3C23CCC3)c1F] |
| 180 | 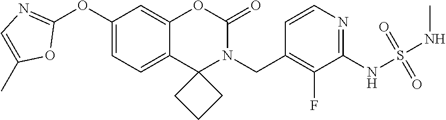 [SMILES: CNS(=O)(=O)Nc1nccc(CN2C(=O)Oc3cc(Oc4ncc(C)o4)ccc3C23CCC3)c1F] |
| 181 | 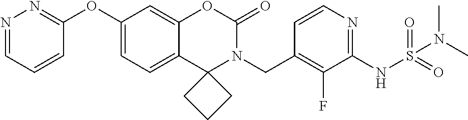 [SMILES: CN(C)S(=O)(=O)Nc1nccc(CN2C(=O)Oc3cc(Oc4cccnn4)ccc3C23CCC3)c1F] |
| 182 | 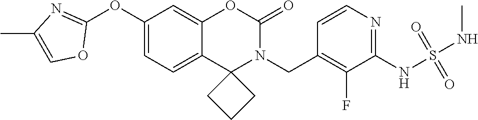 |
| 183 | 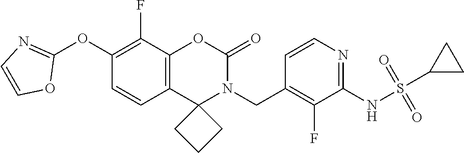 [SMILES: O=C1Oc2c(ccc(Oc3ncco3)c2F)C2(CCC2)N1Cc1ccnc(NS(=O)(=O)C2CC2)c1F] |
| 184 | 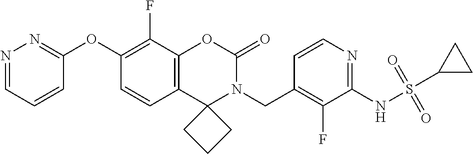 [SMILES: O=C1Oc2c(ccc(Oc3cccnn3)c2F)C2(CCC2)N1Cc1ccnc(NS(=O)(=O)C2CC2)c1F] |
| 185 | 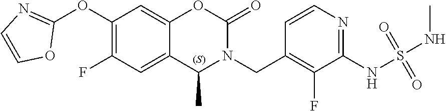 |
| | or |
| | 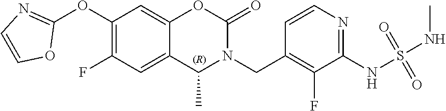 |
| 186 | 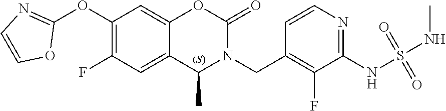 |
| | or |
| | 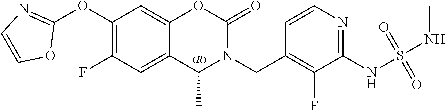 |
| 187 | 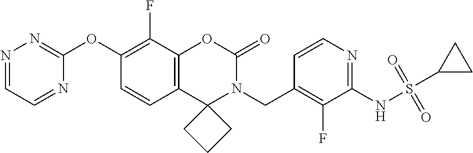 [SMILES: O=C1Oc2c(ccc(Oc3nccnn3)c2F)C2(CCC2)N1Cc1ccnc(NS(=O)(=O)C2CC2)c1F] |
| 188 | 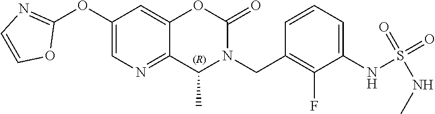 |
| | or |
| | 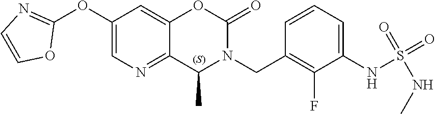 |
| 189 | 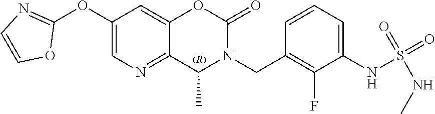 |
| | or |
| | 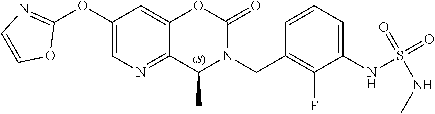 |
| 190 | 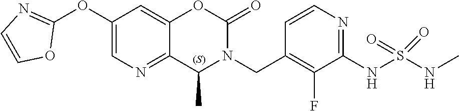 |
| | or |
| | 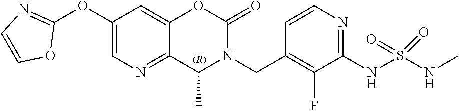 |
| 191 | 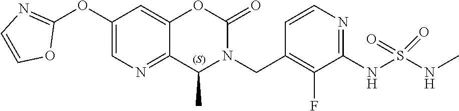 |
| | or |
| | 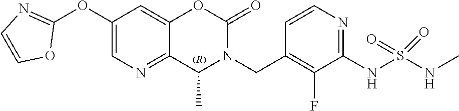 |
| 192 | 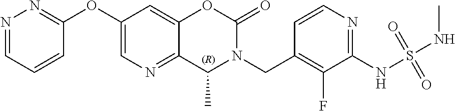 |
| | or |
| | 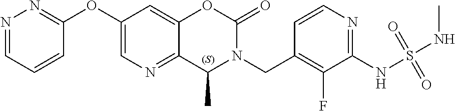 |
| 193 | 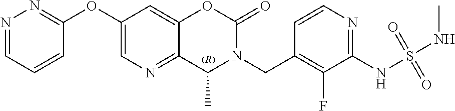 |
| | or |
| | 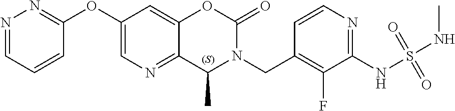 |
| 194 | 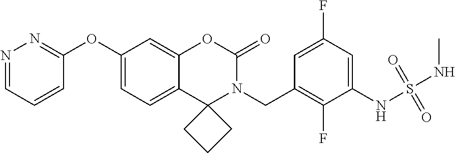 [SMILES: CNS(=O)(=O)Nc1cc(F)cc(CN2C(=O)Oc3cc(Oc4cccnn4)ccc3C23CCC3)c1F] |
| 195 | 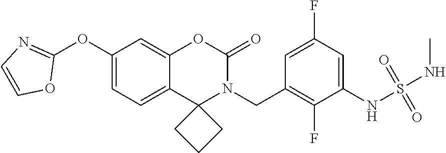 [SMILES: CNS(=O)(=O)Nc1cc(F)cc(CN2C(=O)Oc3cc(Oc4ncco4)ccc3C23CCC3)c1F] |
| 196 | 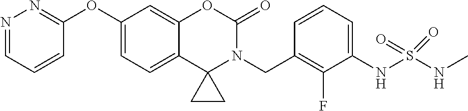 [SMILES: CNS(=O)(=O)Nc1cccc(CN2C(=O)Oc3cc(Oc4cccnn4)ccc3C23CC3)c1F] |
| 197 | 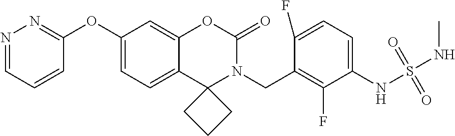 [SMILES: CNS(=O)(=O)Nc1ccc(F)c(CN2C(=O)Oc3cc(Oc4cccnn4)ccc3C23CCC3)c1F] |
| 198 | 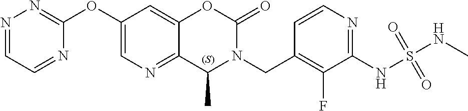 |
| | or |
| | 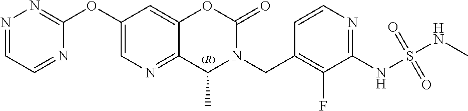 |
| 199 | 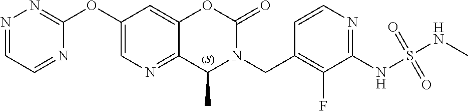 |
| | or |
| | 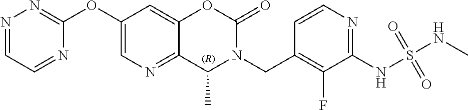 |
| 200 | 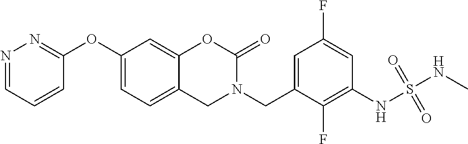 [SMILES: CNS(=O)(=O)Nc1cc(F)cc(CN2Cc3ccc(Oc4cccnn4)cc3OC2=O)c1F] |
| 201 | 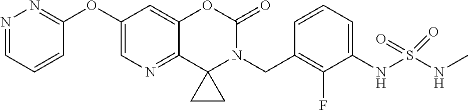 [SMILES: CNS(=O)(=O)Nc1cccc(CN2C(=O)Oc3cc(Oc4cccnn4)cnc3C23CC3)c1F] |
| 202 | 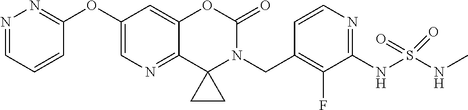 [SMILES: CNS(=O)(=O)Nc1nccc(CN2C(=O)Oc3cc(Oc4cccnn4)cnc3C23CC3)c1F] |

### III. Compound Synthesis

### General Synthetic Schemes

Exemplary methods for preparing compounds described herein are illustrated in the following synthetic schemes. These schemes are given for the purpose of illustrating the scope of the disclosure, and should not be regarded in any manner as limiting the scope or the spirit of the claimed invention.

The synthetic route illustrated in Scheme 1 depicts an exemplary procedure for preparing a compound of Formula (IA), where R¹ and R² are both hydrogen.

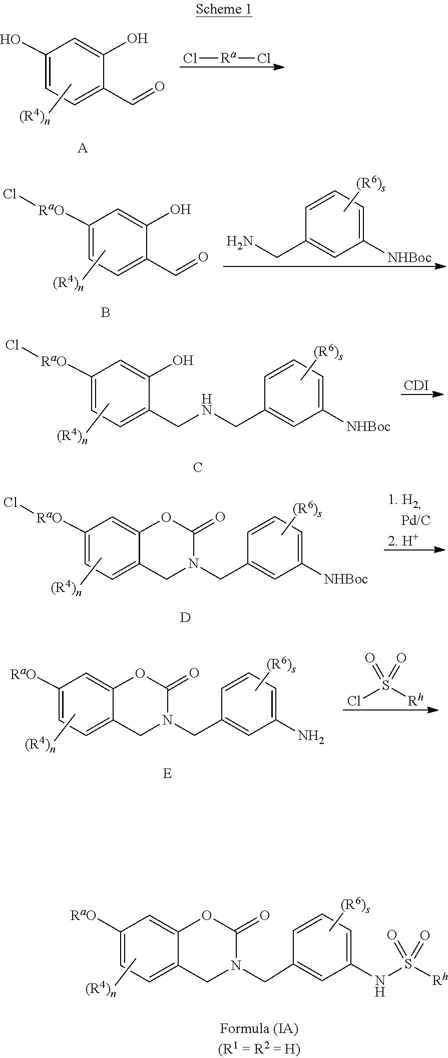

In the first step, optionally substituted 2,4-dihydroxybenzaldehyde A is reacted regioselectively with di-chloro agent Cl—R^{a}—Cl to give ether B. Alternatively, mono-chloro agent R^{a}—Cl may be used. This step may be modified depending on the particular R^{a} group. In the second step, ether B is reacted under standard reductive amination conditions with a substituted amine (generally available commercially) to give compound C₃₋₆. In the third step, carbamate D is formed through reaction with carbonyl di-imdazole. In the fourth step, the chloride on R^{a} group is removed via hydrogenolysis (if mono-chloro agent R^{a}—Cl is used in the first step, this step is omitted), and the Boc protecting group is removed under acidic conditions to provide compound E. In the last step, a reaction with a chlorosulfonamide agent yields a compound of Formula (IA).

The synthetic route illustrated in Scheme 1A depicts an exemplary procedure for preparing a compound of Formula (IA), where at least one of R¹ and R² is not hydrogen.

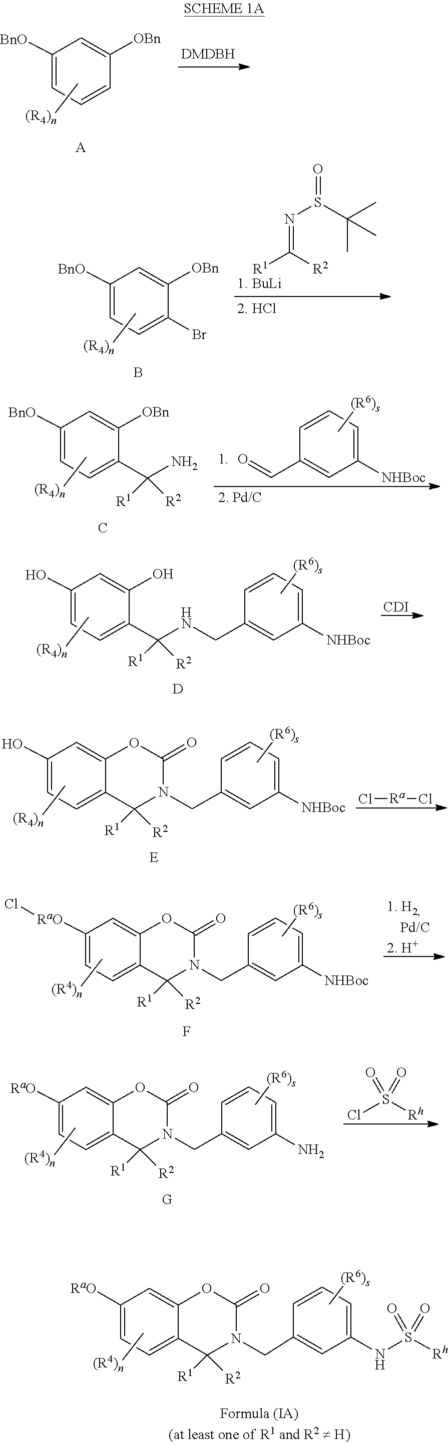

In the first step, an optionally substituted 2,4-dibenzyloxybenzene A is regioselectively reacted with dimethylbromohydantoin (DMDBH) to give the brominated compound B. In the second step, treatment of B with a n-butyllithium (or suitable lithiation reagent) provides the intermediate organolithium reagent, which is reacted with an appropriately substituted sulfenamide and then treated with aqueous HCl to provide the amine C₃₋₆. In the third step, standard reductive amination conditions with an appropriately substituted benzaldehyde provides D. In the fourth step, compound D is treated with carbonyl di-imadazole to form the carbamate E. In the fifth step, compound E is reacted with R^{a}—Cl or Cl—R^{a}—Cl agent to provide compound F. This step may be modified depending on the particular R^{a} group. In the sixth step, if Cl—R^{a}—Cl agent is used in the previous step, the remaining chloride is removed via hydrogenolysis, and the Boc protecting group is removed under acidic conditions to provide compound G. In the last step, a reaction with a chlorosulfonamide agent yields a compound of Formula (IA).

The synthetic route illustrated in Scheme 2 depicts an exemplary procedure for preparing a compound of Formula (IB), where R¹ and R² are both hydrogen and t is 1.

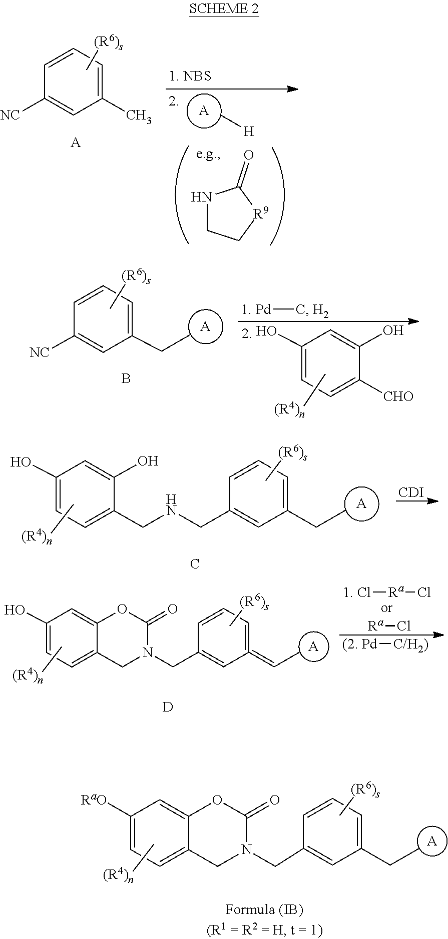

In the first step, optionally substituted 3-methylbenzonitrile A is reacted with N-bromosuccinimide to provide an intermediate bromide, which is reacted with ring A (which contains an amide, urea, or carbamate) to form compound B. This step may be modified if ring A does not have an amide, urea, or carbamate. In the second step, the nitrile moiety of compound B is reduced under standard hydrogenation conditions, and the resultant amine is reacted via reductive amination with 2,4-dihydroxybenzaldehyde derivative to yield amine C₃₋₆. In the third step, carbamate D is formed through reaction of the amine and phenol with carbonyl di-imdazole. In the last step, the remaining phenolic group is reacted with R^{a}—Cl or Cl—R^{a}—Cl agent. This step may be modified depending on the particular R^{a} group. If Cl—R^{a}—Cl agent is used, the remaining chloride is removed via hydrogenolysis to give a compound of Formula (IB). Ring A may be replaced with an acyclic amide, urea, or carbamate to prepare an acyclic analog.

The synthetic route illustrated in Scheme 2A depicts an exemplary procedure for preparing a compound of Formula (IB), where R¹ and R² are both hydrogen and t is 0.

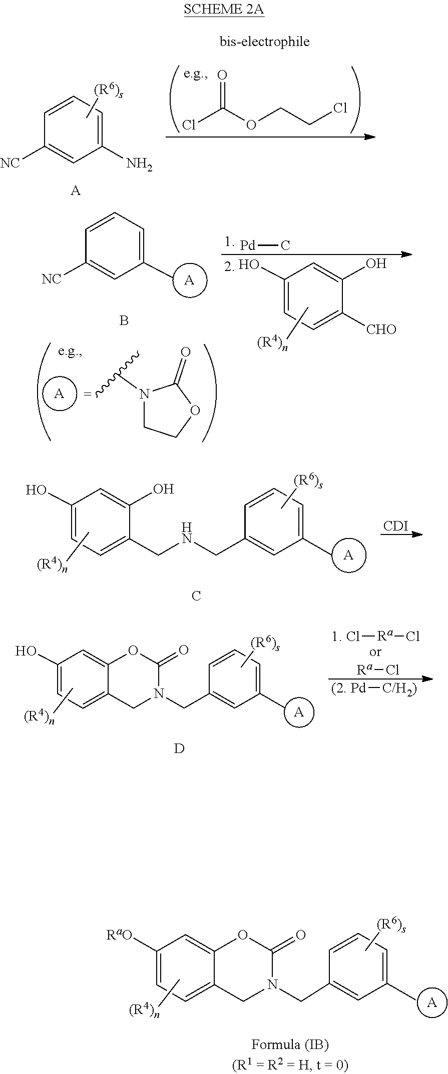

In the first step, optionally substituted 3-aminobenzonitrile A is reacted with a bis-electrophile that forms ring A with the amine to provide compound B. This step may be modified based on the structure of ring A. In the second step, the nitrile moiety of compound B is reduced under standard hydrogenation conditions, and the resultant amine is reacted via reductive amination with 2,4-dihydroxybenzaldehyde derivative to yield amine C. In the third step, carbamate D is formed through reaction of the amine and phenol with carbonyl di-imdazole. In the last step, the remaining phenolic group is reacted with R^{a}—Cl or Cl—R^{a}—Cl agent. This step may be modified depending on the particular R^{a} group. If Cl—R^{a}—Cl agent is used, the remaining chloride is removed via hydrogenolysis to give a compound of Formula (IB).

The synthetic route illustrated in Scheme 2B depicts an exemplary procedure for preparing a compound of Formula (IB), where at least one of R¹ and R² are both hydrogen and t is 1.

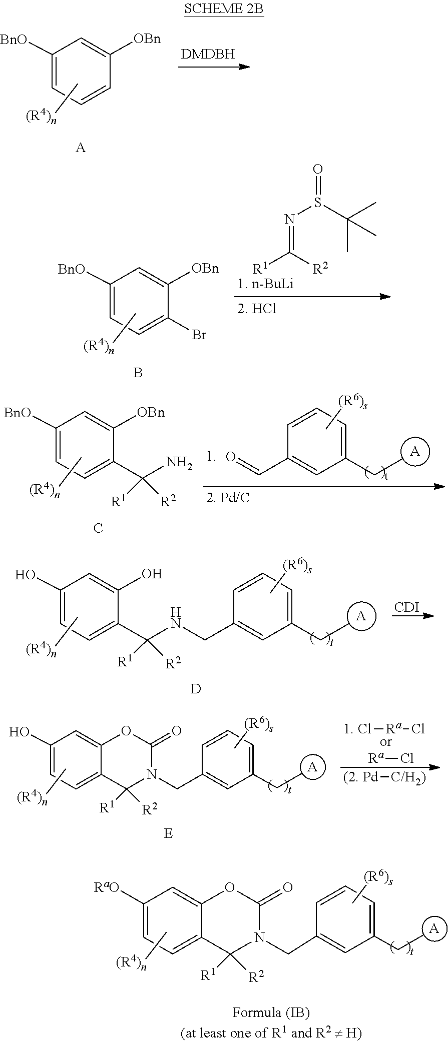

The preparation of compound C is as described in Scheme 1B. Standard reductive amination of compound C with an appropriately substituted benzaldehyde (prepared separately) provides compound D. Carbamate E is formed through reaction of the amine and phenol with carbonyl di-imdazole. Then carbamate E is reacted with R^{a}—Cl or C₁—R″—C₁ agent. This step may be modified depending on the particular R^{a} group. Finally, if Cl—R^{a}—Cl agent is used in the previous step, the remaining chloride is removed via hydrogenolysis to yield a compound of Formula (IB).

In addition to the general synthetic schemes, specific syntheses of the compounds disclosed herein are set forth in the various Examples.

### IV. Pharmaceutical Compositions and Routes of Administration

Compounds provided in accordance with the present disclosure, e.g., a compound of Formula (I), (e.g., (I-1), (IA), (IA-1), (IB), (IB3-1), (IC), (IC-1), (IC-2), (ID), (IE), (IF), (IF-1), (IF-2), (IG), (IG-1), or (IG-2)) are usually administered in the form of pharmaceutical compositions. Provided herein are pharmaceutical compositions that contain, as the active ingredient, one or more of the compounds described, or a pharmaceutically acceptable salt or ester thereof, and one or more pharmaceutically acceptable excipients, carriers, including inert solid diluents and fillers, diluents, including sterile aqueous solution and various organic solvents, permeation enhancers, solubilizers and adjuvants. The pharmaceutical compositions may be administered alone or in combination with other therapeutic agents. Such compositions are prepared in a manner well known in the pharmaceutical art (see, e.g., Adeboye Adejare, Remington: The Science and Practice of Pharmacy (23d ed. 2020)).

In certain embodiments, a pharmaceutical composition may contain formulation materials for modifying, maintaining or preserving, for example, the pH, osmolarity, viscosity, clarity, color, isotonicity, odor, sterility, stability, rate of dissolution or release, adsorption or penetration of the composition. In such embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; and/or excipients (see Adeboye Adejare (2020) supra).

The pharmaceutical compositions may be administered in either single or multiple doses by any of the accepted modes of administration of agents having similar utilities, including, orally, topically, by rectal, buccal, intranasal or transdermal routes, intravenously, intraperitoneally, parenterally, intramuscularly, subcutaneously, or as an inhalant, or via an impregnated or coated device, e.g., a stent.

One mode for administration is parenteral, particularly by injection. The forms in which the novel compounds or compositions described herein may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles. Aqueous solutions in saline are also conventionally used for injection, but less preferred in the context of the present invention. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Sterile injectable solutions are prepared by incorporating a compound according to the present invention in the required amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Oral administration is another route for administration of compounds in accordance with the invention. Administration may be via capsule or enteric coated tablets, or the like. In making the pharmaceutical compositions that include at least one compound described herein, the active ingredient is usually diluted by an excipient and/or enclosed within such a carrier that can be in the form of a capsule, sachet, paper or other container. When the excipient serves as a diluent, it can be in the form of a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include: lubricating agents such as talc, magnesium stearate, and mineral oil; wetting agents; emulsifying and suspending agents; preserving agents such as methyl and propylhydroxy-benzoates; sweetening agents; and flavoring agents.

The compounds or compositions described herein can be formulated so as to provide quick, sustained or delayed release of the active ingredient after administration to the patient by employing procedures known in the art. Controlled release drug delivery systems for oral administration include osmotic pump systems and dissolutional systems containing polymer-coated reservoirs or drug-polymer matrix formulations. Examples of controlled release systems are given in U.S. Pat. Nos. 3,845,770; 4,326,525; 4,902,514; and 5,616,345. Similarly, the compounds or compositions described herein can be delivered by transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art (see, e.g., U.S. Pat. Nos. 5,023,252, 4,992,445 and 5,001,139). Such patches may be constructed for continuous, pulsatile, or on demand delivery of pharmaceutical agents.

The compositions are preferably formulated in a unit dosage form. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient (e.g., a tablet, capsule, ampoule). The compounds are generally administered in a pharmaceutically effective amount. For oral administration, each dosage unit may contain from 1 mg to 2 g (e.g., 1 mg to 1 g, 1 mg to 500 mg, 1 mg to 100 mg) of a compound described herein, and for parenteral administration, from 0.1 to 700 mg (e.g., from 0.1 mg to 300 mg, from 0.1 mg to 100 mg, from 0.1 mg to 10 mg) of a compound described herein. The reference to compound includes the compound either alone or in combination with a pharmaceutically acceptable salt. It will be understood, however, that the amount of the compound actually administered usually will be determined by a physician, in the light of the relevant circumstances, including the condition to be treated, the chosen route of administration, the actual compound administered and its relative activity, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a compound of the present invention. When referring to these preformulation compositions as homogeneous, it is meant that the active ingredient is dispersed evenly throughout the composition so that the composition may be readily subdivided into equally effective unit dosage forms such as tablets, pills and capsules.

The tablets or pills of the present invention may be coated or otherwise compounded to provide a dosage form affording the advantage of prolonged action, or to protect from the acid conditions of the stomach. For example, the tablet or pill can comprise an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Preferably, the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a facemask tent, or intermittent positive pressure breathing machine. Solution, suspension, or powder compositions may be administered, preferably orally or nasally, from devices that deliver the formulation in an appropriate manner.

In some embodiments, a pharmaceutical composition comprising a disclosed compound, or pharmaceutically acceptable salt thereof, and a pharmaceutically acceptable carrier.

### V. Methods of Treatment

The compounds and compositions herein can be used in a method of modulating the interaction between RAF and MEK in a RAF-MEK complex (e.g., BRAF and MEK in a BRAF-MEK complex). The method comprises contacting BRAF, MEK or a combination of BRAF and MEK with an effective amount of a compound disclosed therein or a pharmaceutically acceptable salt thereof, or a pharmaceutical composition disclosed herein thereby to modulate the interaction between BRAF and MEK. The modulation can involve stabilizing or destabilizing the interaction between RAF and MEK in a RAF-MEK complex, e.g., BRAF and MEK in a BRAF-MEK complex.

The compounds and compositions described herein can be used to treat a disease or condition associated with MAPK signaling pathway in a subject in need thereof. The method comprises administering to the subject a therapeutically effective amount of a compound disclosed herein or a pharmaceutically acceptable salt thereof, or a pharmaceutical composition disclosed herein. In certain embodiments the disease or condition is cancer.

The compounds and compositions described above and herein can be used to treat an abnormal cell growth in a subject in need thereof. Abnormal cell growth, as used herein and unless otherwise indicated, refers to cell growth that is independent of normal regulatory mechanisms (e.g., loss of contact inhibition). This includes the abnormal growth of: (1) tumor cells (tumors) whose growth is mediated, e.g., by aberrant MAPK Pathway Signaling; (2) benign and malignant cells of other proliferative diseases, e.g., in which aberrant RAF-MEK complex, e.g., BRAF-MEK, complex mediated signaling occurs; (3) any tumors that proliferate, e.g., by RAF-MEK complex, e.g., BRAF-MEK complex, signaling; (4) tumor proliferation, e.g., by aberrant upstream dysregulation of MAPK pathway including EGFR or RAS proteins; and (5) neoplastic disorders e.g., in which aberrant RAF-MEK complex, e.g., BRAF-MEK complex, signaling occurs.

Abnormal cell growth can refer to cell growth of epithelial cells (e.g., carcinomas, adenocarcinomas), mesenchymal cells (e.g., sarcomas (e.g., Ewing's sarcoma)), hematopoetic cells (e.g., lymphomas, leukemias, myelodysplasias) or other cells (e.g., melanoma, mesothelioma, and other tumors of unknown origin). A “neoplastic disorder” is a disease or disorder characterized by cells that have the capacity for autonomous growth or replication, e.g., an abnormal state or condition characterized by proliferative cell growth. An abnormal mass of tissue as a result of abnormal cell growth or division, or a neoplasm, can be benign, pre-malignant or malignant. Exemplary neoplastic disorders include: carcinoma, sarcoma, metastatic disorders (e.g., tumors arising from prostate, colon, lung, breast and liver origin), hematopoietic neoplastic disorders, e.g., leukemias, metastatic tumors. Treatment with the compound may be in an amount effective to ameliorate at least one symptom of the neoplastic disorder, e.g., reduced cell proliferation, reduced tumor mass, etc.

The methods and compositions described herein may be useful in the prevention and treatment of a cancer, including, e.g., solid tumors, soft tissue tumors, and metastases thereof. The disclosed methods are also useful in treating non-solid cancers. Exemplary solid tumors include malignancies (e.g., sarcomas, adenocarcinomas, and carcinomas) of the various organ systems, such as those of lung, breast, lymphoid, gastrointestinal (e.g., colon, rectum, or colorectum), brain, pancreas, genitourinary tracts (e.g., renal, urothelial, or testicular tumors), pharynx, prostate, and ovary. Exemplary adenocarcinomas include colorectal cancers, renal-cell carcinoma, liver cancer (e.g., hepatocellular carcinoma), lung cancer (e.g., non-small cell lung cancer (NSCLC)), pancreatic (e.g., metastatic pancreatic adenocarcinoma) and cancer of the small intestine. In some embodiments, the cancer can be colorectal cancer, NSCLC, or pancreatic cancer. In certain embodiments, the cancer may include brain metastasis.

The cancer can include mesothelioma; neurofibromatosis; e.g., neurofibromatosis type 2, neurofibromatosis type 1, renal cancer; lung cancer, non-small cell lung cancer; liver cancer; thyroid cancer; ovarian; breast cancer; a nervous system tumor; schwannoma; meningioma; schwannomatosis; neuroma acoustic; adenoid cystic carcinoma; ependymoma; ependymal tumors, or any other tumor which exhibits decreased merlin expression and/or mutation, and/or deletion and/or promotor hypermethylation of the NF-2 gene. In some embodiments, the cancer is renal cancer.

The cancer can include cancers characterized as comprising cancer stem cells, cancer associated mesenchymal cells, or tumor initiating cancer cells. The cancer can include cancers that have been characterized as being enriched with cancer stem cells, cancer associated mesenchymal cells, or tumor initiating cancer cells (e.g., a tumor enriched with cells that have undergone an epithelial-to-mesenchymal transition or a metastatic tumor).

The cancer can be a primary tumor, i.e., located at the anatomical site of tumor growth initiation. The cancer can also be metastatic, i.e., appearing at least a second anatomical site other than the anatomical site of tumor growth initiation. The cancer can be a recurrent cancer, i.e., cancer that returns following treatment, and after a period of time in which the cancer was undetectable. The recurrent cancer can be anatomically located locally to the original tumor, e.g., anatomically near the original tumor; regionally to the original tumor, e.g., in a lymph node located near the original tumor; or distantly to the original tumor, e.g., anatomically in a region remote from the original tumor. In some embodiments, the cancer can be brain or cerebrospinal fluid (CSF)-related metastasis.

The cancer can also include for example, but is not limited to, epithelial cancers, breast, lung, pancreatic, colorectal (e.g., metastatic colorectal, e.g., metastatic KRAS mutated), prostate, head and neck, melanoma (e.g., NRAS mutated locally advanced or metastatic malignant cutaneous melanoma), acute myelogenous leukemia, and glioblastoma. Exemplary breast cancers include triple negative breast cancer, basal-like breast cancer, claudin-low breast cancer, invasive, inflammatory, metaplastic, and advanced HER-2 positive or ER-positive cancers resistant to therapy.

In some embodiments, the cancer can also include a cancer characterized as having a RAF mutation. In some embodiments, the cancer can also include a cancer characterized as having a KRAS mutation, an NRAS mutation, or a HRAS mutation. In some embodiments, the cancer may also be characterized as having EGFR overexpression.

The cancer can also include lung adenocarcinoma, colorectal cancer (CRC), uveal melanoma, ovarian cancer, uterine endometrioid carcinoma, bladder urothelial carcinoma, breast invasive lobular carcinoma, cervical squamous cell carcinoma, cutaneous melanoma, endocervical adenocarcinoma, hepatocellular carcinoma, pancreatic adenocarcinoma, biphasic type pleural mesothelioma, renal clear cell carcinoma, renal clear cell carcinoma, stomach adenocarcinoma, tubular stomach adenocarcinoma, head and neck squamous cell carcinoma, uterine carcinosarcoma, or uterine malignant mixed Mullerian tumor.

In some embodiments, the cancer is unresectable or metastatic melanoma, melanoma with lymph node involvement or metastatic disease who have undergone complete resection, metastatic non-small cell lung cancer and progression on or after platinum-based chemotherapy, metastatic small cell lung cancer with progression after platinum-based chemotherapy and at least one other line of therapy, advanced renal cell carcinoma who have received prior anti angiogenic therapy, advanced renal cell carcinoma, classical Hodgkin lymphoma, recurrent or metastatic squamous cell carcinoma of the head and neck with disease progression on or after a platinum-based therapy, locally advanced or metastatic urothelial carcinoma, microsatellite instability-high (MSI-H) or mismatch repair deficient (dMMR) metastatic colorectal cancer, or hepatocellular carcinoma.

In some embodiments, the cancer is melanoma, non-small cell lung cancer, small cell lung cancer, head and neck squamous cell cancer, classical Hodgkin lymphoma, primary mediastinal large B-cell lymphoma, urothelial carcinoma, microsatellite instability-high cancer, gastric cancer, esophageal cancer, cervical cancer, hepatocellular carcinoma, merkel cell carcinoma, renal cell carcinoma, or endometrial carcinoma.

Other cancers include but are not limited to, uveal melanoma, brain, abdominal, esophagus, gastrointestinal, glioma, liver, tongue, neuroblastoma, osteosarcoma, ovarian, retinoblastoma, Wilm's tumor, multiple myeloma, skin, lymphoma, blood and bone marrow cancers (e.g., advanced hematological malignancies, leukemia, e.g., acute myeloid leukemia (e.g., primary' or secondary), acute lymphoblastic leukemia, acute lymphocytic leukemia, T cell leukemia, hematological malignancies, advanced myeloproliferative disorders, myelodysplastic syndrome, relapsed or refractory' multiple myeloma, advanced myeloproliferative disorders), retinal, bladder, cervical, kidney, endometrial, meningioma, lymphoma, skin, uterine, lung, non small cell lung, nasopharyngeal carcinoma, neuroblastoma, solid tumor, hematologic malignancy, squamous cell carcinoma, testicular, thyroid, mesothelioma, brain vulval, sarcoma, intestine, oral, endocrine, salivary, spermatocyte seminoma, sporadic medullary thyroid carcinoma, non-proliferating testes cells, cancers related to malignant mast cells, non-Hodgkin's lymphoma, and diffuse large B cell lymphoma.

In some embodiments, the tumor is a solid tumor. In some embodiments, the solid tumor is locally advanced or metastatic. In some embodiments, the solid tumor is refractory (e.g., resistant) after standard therapy.

Methods described herein can reduce, ameliorate or altogether eliminate the disorder, and/or its associated symptoms, to keep it from becoming worse, to slow the rate of progression, or to minimize the rate of recurrence of the disorder once it has been initially eliminated (e.g., to avoid a relapse). A suitable dose and therapeutic regimen may vary depending upon the specific compounds, combinations, and/or pharmaceutical compositions used and the mode of delivery of the compounds, combinations, and/or pharmaceutical compositions. In some embodiments, the method increases the average length of survival, increases the average length of progression-free survival, and/or reduces the rate of recurrence, of subjects treated with the combinations described herein in a statistically significant manner.

In some embodiments, the cancer is lung cancer (e.g., non-small cell lung cancer CNSCLC), e.g., KRAS mutant NSCLC, metastatic cancer), bone cancer, pancreatic cancer, skin cancer, cancer of the head or neck, cutaneous or intraocular melanoma, uterine cancer, ovarian cancer (e.g., unresectable low-grade ovarian, advanced or metastatic ovarian cancer), rectal cancer, cancer of the anal region, stomach cancer, colon cancer, breast cancer (e.g., triple-negative breast cancer (e.g., breast cancer which does not express the genes for the estrogen receptor, progesterone receptor, and Her2/neu)), uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary' adenoma, mesothelioma (e.g., malignant pleural mesothelioma, e.g., surgical resectable malignant pleural mesothelioma) or a combination of one or more of the foregoing cancers.

### Examples

In order that the invention described herein may be more fully understood, the following examples are set forth. The synthetic and biological examples described in this application are offered to illustrate the compounds, pharmaceutical compositions and methods provided herein and are not to be construed in any way as limiting their scope.

The compounds provided herein can be prepared from readily available starting materials using the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimal reaction conditions may vary with the particular reactants or solvent used, but such conditions can be determined by one skilled in the art by routine optimization.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in T. W. Greene and P. G. M. Wuts, Protecting Groups in Organic Synthesis, Second Edition, Wiley, New York, 1991, and references cited therein.

The compounds provided herein may be isolated and purified by known standard procedures. Such procedures include recrystallization, filtration, flash chromatography, trituration, high pressure liquid chromatography (HPLC), or supercritical fluid chromatography (SFC). Note that flash chromatography may either be performed manually or via an automated system. The compounds provided herein may be characterized by known standard procedures, such as nuclear magnetic resonance spectroscopy (NMR) or liquid chromatography mass spectrometry (LCMS). NMR chemical shifts are reported in part per million (ppm) and are generated using methods well known to those of skill in the art.

### LIST OF ABBREVIATIONS

ACN Acetonitrile
AcOH Acetic acid
ADP Adenosine diphosphate
AIBN Azobisisobutyronitrile
aq. Aqueous
ATP Adenosine triphosphate
BBr₃ Boron tribromide
BF₃AcOH Boron trifluoride acetic acid complex
BF₃·Et₂O Boron trifluoride etherate
BnBr Benzyl bromide
BnOH Benzyl alcohol
B₂Pin₂ Bis(pinacolato)diboron
Boc-NH₂ tert-Butyl carbamate
(Boc)₂O Di-tert-butyl dicarbonate
BRAF B-Raf proto-oncogene
CBr₄ Carbon tetrabromide
CBz-C₁ Benzyl chloroformate
CCl₄ Carbon tetrachloride
CICOOBu Isobutyl chloroformate
CDI 1,1′-Carbonyldiimidazole
CHAPS 3-{Dimethyl[3-(3α,7α,12α-trihydroxy-5β-cholan-24-amido)propyl]azaniumyl}propane-1-sulfonate
CO₂ Carbon dioxide
(COCI)₂ Oxalyl chloride
Cs₂CO₃ Cesium carbonate
C-term C-terminus
Cu Copper
CuCl Copper(I) chloride
CuI Copper(I) iodide
d Day(s)
DBDMH 1,3-Dibromo-5,5-dimethylhydantoin (1,3-Dibromo-5,5-dimethylimidazolidine-2,4-dione)
DMEDA N,N′-Dimethylethylenediamine
DCE Dichloroethane
DCM Dichloromethane
DEA Diethylamine
DIAD Diisopropyl azodicarboxylate
DIBAL (or DIBAL-Diisobutylaluminum hydride H)
DIPEA Diisopropylethylamine
DMAP 4-Dimethylaminopyridine
DMEDA 1,2-Dimethylethylenediamine
DMF N,N′-dimethylformamide
DMF-d₇ Deuterated N,N′-dimethylformamide
DMP Dess-Martin periodinane
DMSO Dimethyl sulfoxide
DMSO-d₆ Deuterated dimethyl sulfoxide
DPPA Diphenylphosphoryl azide
DTT Dithiothreitol
EC₅₀ Half maximal effective concentration
ERK Extracellular signal-regulated kinase
ES Electrospray ionization
EtMgBr Ethylmagnesium bromide
EtOAc Ethyl acetate
Ex/Em Excitation/emission ratio
FeCl₃ Iron(III) chloride
FRET Fluorescence energy transfer
GST Glutathione S-transferase
h Hour; Hours
H₂ Hydrogen
H₂O Water
HATU Hexafluorophosphate azabenzotriazole tetramethyl uronium
HCHO Formaldehyde
HCl Hydrogen chloride
HClO₄ Perchloric acid
HEPES N-2-hydroxyethylpiperazine-N′-2-ethanesulfonic acid
His Histidine
HPLC High-performance liquid chromatography
IC₅₀ Half-maximal inhibitory concentration
ICI Iodine chloride
Im Imidazole
K₂CO₃ Potassium carbonate
K₃PO₄ Tripotassium phosphate
KI Potassium iodide
KOAc Potassium acetate
LAH Lithium aluminum hydride
LDA Lithium diisopropylamide
LiAIH₄ Lithium aluminum hydride
LiCl Lithium chloride
LiOH Lithium hydroxide
m/z Mass-to-charge ratio
Mel Methyl iodide
MEK Mitogen-activated protein kinase kinase
MeMgBr Methyl magnesium bromide
MeNH₂ Methylamine
MeOH Methanol
MgCl₂ Magnesium chloride
min Minutes
MOMCl Chloromethyl methyl ether
N Normal (molarity)
NaN₃ Sodium azide
NaBH₄ Sodium borohydride
NaBH₃CN Sodium cyanoborohydride
NaCl Sodium chloride
NaBD₄ Sodium borodeuteride
NaH Sodium hydride
NaHCO₃ Sodium bicarbonate
NaIO₄ Sodium periodate
NaOMe Sodium methoxide
Na₂SO₄ Sodium sulfate
Na2S203 Sodium thiosulfate
NBS N-bromosuccinimide
n-BuLi n-butyllithium
NH₃ Ammonia
NH₄C₁ Ammonium chloride
NH(Me)₂ Dimethylamine
NMP N-methyl-2-pyrrolidone
N-term N-terminus
OSO₄ Osmium tetroxide
Pd/C Palladium on carbon
PdCl₂(PPh₃)₂ Bis(triphenylphosphine)palladium(II) dichloride
Pd₂(dba)₃ Tris(dibenzylideneacetone)dipalladium(O)
Pd(dppf)Cl₂ [1, 1′-Bis(diphenylphosphino)ferrocene]dichloropalladium(IT)
Pd(dppf)Cl₂DCM [1,1′-Bis(diphenylphosphino)ferrocene]dichloropalladium(II) complex with dichloromethane
Pd(dppf)Cl₂—CH₂Cl₂ [1,1′-Bis(diphenylphosphino)ferrocene]dichloropalladium(II) complex with dichloromethane
Pd(OAc)₂ Palladium acetate
PMA Phorbol myristate acetate
PMB-C₁ para-Methoxybenzyl chloride
POC13 Phosphoryl trichloride
PPh₃ Triphenylphosphine
PPTS Pyridinium p-toluenesulfonate
Py Pyridine
rt Room temperature
SnCl₂·2H₂O Stannous chloride dihydrate
SOC12 Thionyl chloride
STAB Sodium triacetoxyborohydride
TBAAc Tetrabutylammonium acetate
TBAF Tetrabutylammonium fluouride
TBDMS-C₁ tert-Butyldimethylsilyl chloride
TC Tissue culture
TEA Triethylamine
TFA Trifluoroacetic acid
THE Tetrahydrofuran
Ti(OⁱPr)₄ Titanium(IV) isopropoxide
TLC Thin-layer chromatography
TMSCF₃ Trimethyl(trifluoromethyl)silane
TMSN₃ Trimethylsilyl azide
T₃P Propanephosphonic acid anhydride
TR-FRET Time-resolved fluorescence energy transfer
Tris Tris(hydroxymethyl)aminomethane

### INTERMEDIATE PREPARATION

### Intermediate 01: Preparation of 2-hydroxy-4-((4-methoxybenzyl)oxy)benzaldehyde (I-01)

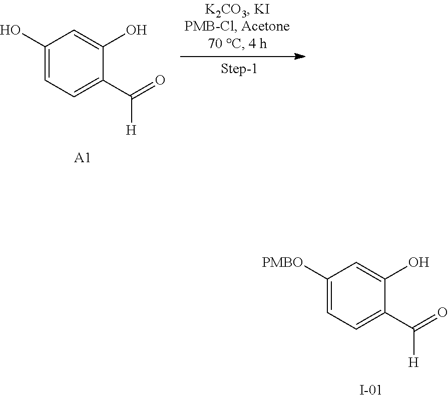

Step-1: Synthesis of 2-hydroxy-4-((4-methoxybenzyl)oxy)benzaldehyde (I-01): To a stirred solution of 2,4-dihydroxybenzaldehyde (A1) (3 g, 21.7 mmol) in acetone (75 mL) was added K₂CO₃ (3.3 g, 23.9 mmol) and potassium iodide (3.61 g, 21.7 mmol) followed by 1-(chloromethyl)-4-methoxybenzene (3.4 g, 21.7 mmol) at room temperature. The reaction mixture was stirred at 70° C. and the reaction progress was monitored by TLC/LCMS. After complete consumption of starting material (2 h), it was cooled to room temperature and water (30 mL) was added to it. Extraction was carried out using EtOAc (30 mL×3); the combined organic extracts were washed with water (50 mL), brine (50 mL), dried over anhydrous Na₂SO₄, filtered and concentrated under reduced pressure. The residue was purified by Combi-Flash (using gradient elution of 0-10% of EtOAc in heptane) to afford desired compound I-01 (4.1 g) as an off white solid. LCMS (ES) m/z: 259.1 [M+H]⁺.

### Intermediate 02: Preparation of 2-(benzyloxy)-4-hydroxybenzaldehyde (I-02)

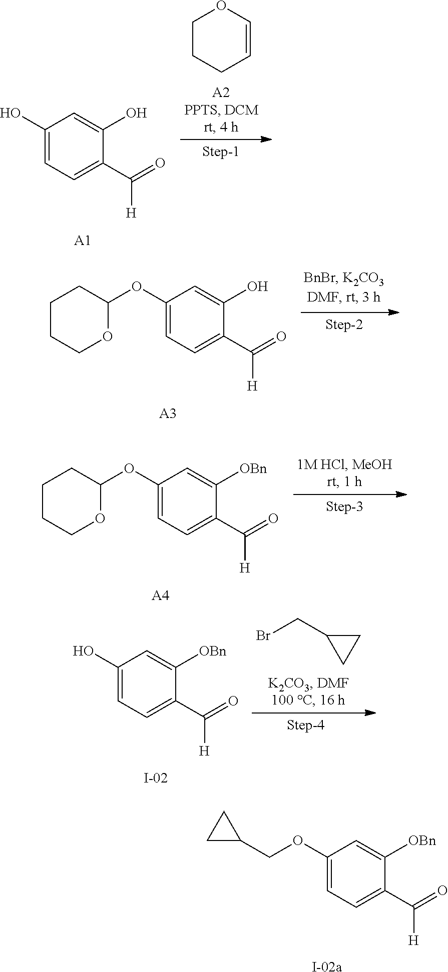

Step-1: Synthesis of 2-hydroxy-4-(oxan-2-yloxy) benzaldehyde (A3): To a stirred solution of 2,4-dihydroxybenzaldehyde (A1) (10 g, 72.4 mmol) in DCM (100 mL) was added PPTS (728 mg, 2.9 mmol) and 3,4-dihydro-2H-pyran (A2) (13.4 g, 159 mmol) at 0° C. The reaction mixture was stirred at room temperature and the reaction progress was monitored by TLC/LCMS. After complete consumption of starting material (4 h), volatiles were removed under reduced pressure and the residue was purified by Combi-Flash (using gradient elution of 0-50% of EtOAc in heptane) to afford the desired compound A3 (15 g) as a colorless liquid. LCMS (ES) m/z: 221.2 [M−H]⁺.

Step-2: Synthesis of 2-(benzyloxy)-4-(oxan-2-yloxy) benzaldehyde (A4): To a stirred solution of 2-hydroxy-4-(oxan-2-yloxy) benzaldehyde (A3) (15 g, 67.5 mmol) in DMF (150 mL) was added K₂CO₃ (18.7 g, 135 mmol) and benzyl bromide (13.9 g, 81 mmol) at 0° C. The reaction mixture was stirred at room temperature and the reaction progress was monitored by TLC/LCMS. After complete consumption of starting material (3 h), water (200 mL) was added to it and extraction was carried out using EtOAc (3×100 mL). The combined organic extracts were washed with cold water (2×200 mL), brine solution (2×200 mL), dried over anhydrous Na₂SO₄, filtered and concentrated under reduced pressure. The residue was purified by Combi-Flash (using gradient elution of 0-30% of EtOAc in heptane) to afford the desired compound A4 (16 g) as a viscous liquid. LCMS (ES) m/z: 313.3 [M+H]⁺.

Step-3: Synthesis of 2-(benzyloxy)-4-hydroxybenzaldehyde (I-02): To a stirred solution of 2-(benzyloxy)-4-(oxan-2-yloxy)benzaldehyde (16 g, 51.2 mmol) in MeOH (211 mL) was added 1 M aqueous solution of HCl (2 mL) at 0° C. The reaction mixture was stirred at room temperature and the reaction progress was monitored by TLC/LCMS. After complete consumption of starting material (1 h), water (50 mL) was added to it and extraction was carried out using EtOAc (3×70 mL). The combined organic extracts were washed with brine solution (2×70 mL), dried over anhydrous Na₂SO₄, filtered and concentrated under reduced pressure to afford the desired compound I-02 (12 g) as a pale yellow solid. LCMS (ES) m/z: 229.1 [M+H]⁺.

Step-4: Synthesis of 2-(benzyloxy)-4-(cyclopropylmethoxy)benzaldehyde (I-02a): To a stirred solution of 2-(benzyloxy)-4-hydroxybenzaldehyde (I-02) (0.6 g, 2.63 mmol) in DMF (15 mL) was added K₂CO₃ (545 mg, 3.94 mmol) and (bromomethyl)cyclopropane (461 mg, 3.42 mmol) at room temperature. The reaction mixture was stirred at 100° C. and the reaction progress was monitored by TLC/LCMS analysis. After completion of the reaction (16 h), the reaction mixture was cooled to room temperature and ice-cold water (20 mL) was added to it. Extraction was carried out with EtOAc (3×50 mL); the combined organic extracts were washed with brine solution (2×25 mL), dried over anhydrous Na₂SO₄, filtered and concentrated under reduced pressure. The residue was purified by Combi-Flash (using gradient elution of 0-30% of EtOAc in heptane) to afford desired compound I-02a (580 mg) as a pale-yellow solid. LCMS (ES) m/z: 283.2 [M+H]⁺.

### Intermediate 02b: Preparation of 2-(benzyloxy)-4-(oxetan-3-yloxy)benzaldehyde (I-02b)

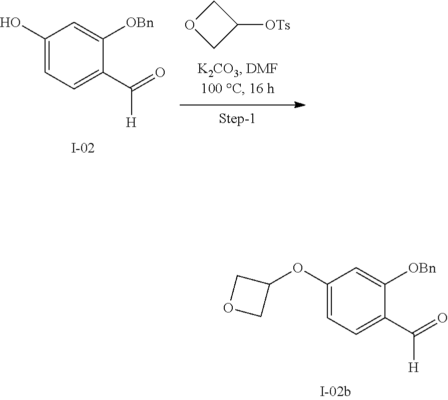 [SMILES: O=Cc1ccc(O)cc1OCc1ccccc1 ; Cc1ccc(S(=O)(=O)OC2COC2)cc1 ; O=Cc1ccc(OC2COC2)cc1OCc1ccccc1]

Step-1: Synthesis of 2-(benzyloxy)-4-(oxetan-3-yloxy)benzaldehyde (I-02b): To a stirred solution of 2-(benzyloxy)-4-hydroxybenzaldehyde (I-02) (0.8 g, 3.5 mmol) in DMF (13.3 mL) was added K₂CO₃ (969 mg, 7.01 mmol) and oxetan-3-yl 4-methylbenzene-1-sulfonate (1.6 g, 7.01 mmol) in room temperature. The reaction mixture was stirred at 100° C. for 16 h. After completion of the reaction, the reaction mixture was cooled to room temperature and quenched with addition of cold water (20 mL). Extraction was carried out using EtOAc (30 mL×3); the combined organic extracts were washed with water (50 mL), brine (50 mL), dried over anhydrous Na₂SO₄, filtered and concentrated under reduced pressure. The residue was purified by Combi-Flash (using gradient elution of 0-10% of EtOAc in heptane) to afford desired compound I-02b (0.8 g) as an off-white solid. LCMS (ES) m/z: 285.2 [M+H]⁺.

### Intermediate 03: Preparation of 3,5-difluoro-2,4-dihydroxybenzaldehyde (1-03)

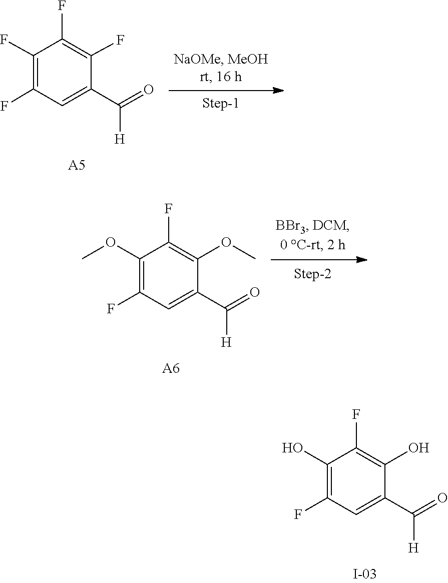

Step-1: Synthesis of 3,5-difluoro-2,4-dimethoxybenzaldehyde (A6): To a stirred solution of 2,3,4,5-tetrafluorobenzaldehyde (A5) (2 g, 11.2 mmol) in MeOH (8 mL) was added 25% of NaOMe in MeOH (8.0 mL, 34.81 mmol) at 0° C. and the reaction mixture was stirred at room temperature. The reaction progress was monitored by TLC/LCMS. After complete consumption of starting material (16 h), the reaction mixture was quenched with addition of water (30 mL) and extraction was carried out using DCM (2×50 mL). The combined organic extracts were washed with brine solution (30 mL), dried over anhydrous Na₂SO₄, filtered and concentrated under reduced pressure. The residue was purified by Combi-Flash (using gradient elution of 0-50% of EtOAc in heptane) to afford the desired compound A6 as an off white solid (1.4 g). LCMS (ES) m/z: 202.9 [M+H]⁺.

Step-2: Synthesis of 3,5-difluoro-2,4-dihydroxybenzaldehyde (1-03): To a stirred solution of 3,5-difluoro-2,4-dimethoxybenzaldehyde (A6) (1.4 g, 6.93 mmol) in anhydrous DCM (10 mL) was added BBr₃ (1.33 mL, 13.9 mmol) at 0° C. and the reaction mixture was allowed to stir at room temperature, while monitoring reaction progress by TLC/LCMS. After complete consumption of starting material (2 h), it was quenched by addition of ice water (20 mL) and extraction was carried out using EtOAc (2×40 mL), The combined organic extracts were washed with brine solution (40 mL), dried over anhydrous Na₂SO₄, filtered and concentrated under reduced pressure. The residue was purified by Combi-Flash (using gradient elution of 0-50% of EtOAc in heptane) to afford desired 1-03 as an off white solid (1 g). LCMS (ES) m/z: 175.0 [M+H]⁺.

### Intermediate 04: Preparation of 3-fluoro-2-hydroxy-4-((4-methoxybenzyl)oxy) benzaldehyde (1-04)

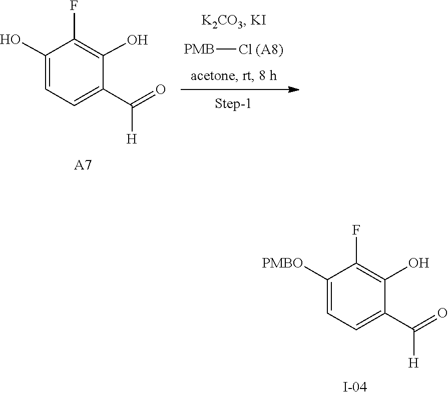

Step-1: Synthesis of 3-fluoro-2-hydroxy-4-[(4-methoxyphenyl)methoxy]benzaldehyde (I-04): Intermediate I-04 (0.5 g) was synthesized by following procedure as described for the synthesis of I-01 (step-1) using 2-fluoro-4,6-dihydroxybenzaldehyde (A7) (0.7 g, 4.48 mmol) as the starting material. LCMS (ES) m/z: 275.0 [M−H]⁺.

### Intermediate 05: Preparation of 5-fluoro-2,4-dihydroxybenzaldehyde (I-05)

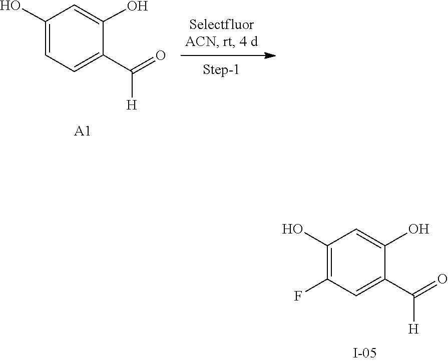 [SMILES: O=Cc1ccc(O)cc1O ; "A1" ; O=Cc1cc(F)c(O)cc1O]

Step-1: Synthesis of 5-fluoro-2,4-dihydroxybenzaldehyde (I-05): To a solution of Selectfluor (32.3 g, 91.2 mmol) in ACN (120 mL) was added 2,4-dihydroxybenzaldehyde (A1) (9.0 g, 65.2 mmol) portion wise and the reaction mixture was stirred at room temperature for 4 days. The reaction progress was monitored by TLC/LCMS. Water (300 mL) was then added to it and extraction was carried out using EtOAc (90 mL×3). The combined organic extracts were washed with brine solution (100 mL), dried over anhydrous Na₂SO₄, filtered and concentrated under reduced pressure. The residue was purified by Combi-Flash (using gradient elution of 0-40% of EtOAc in heptane) followed by reverse phase preparative HPLC purification of the residue obtained [using XSelect CSH C-18 (250 mm×4.6 mm, 5 μm particle size) column, 0.1% formic acid in water and acetonitrile as mobile phase with 1.0 mL/min flow rate] to afford desired I-05 (0.6 g) as a yellow solid. LCMS (ES) m/z: 155.1 (M−H)⁺.

### Intermediate 6: Preparation of 2-fluoro-6-hydroxy-4-((4-methoxybenzyl)oxy)benzaldehyde (1-06)

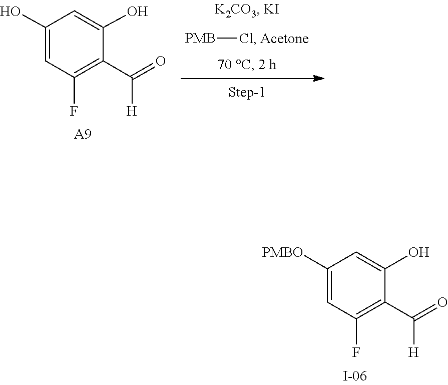

Step-1: Synthesis of 2-fluoro-6-hydroxy-4-((4-methoxybenzyl)oxy)benzaldehyde (1-06): Intermediate 1-06 (0.650 g) was synthesized by following procedure as described for the synthesis of I-01 (step-1) using 2-fluoro-4,6-dihydroxybenzaldehyde (A9) (0.5 g, 3.2 mmol) as the starting material. LCMS (ES) m/z: 277.1 [M+H]⁺.

### Intermediate 7: Preparation of 5-chloro-2-hydroxy-4-((4-methoxybenzyl)oxy) benzaldehyde (I-07)

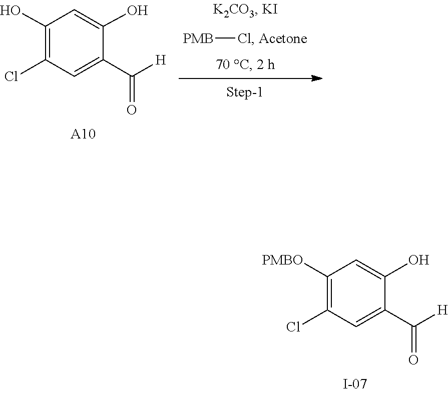

Step-1: Synthesis of 5-chloro-2-hydroxy-4-((4-methoxybenzyl)oxy)benzaldehyde (I-07): Intermediate 1-07 (1.3 g) was synthesized by following procedure as described for the synthesis of 1-01 (step-1) using 5-chloro-2,4-dihydroxybenzaldehyde (A10) (2 g, 11.6 mmol) as the starting material. LCMS (ES) m/z: 291.0 [M−H]⁺.

### Intermediate 8: Preparation of 2-hydroxy-4-((4-methoxybenzyl)oxy)-5-methyl benzaldehyde (I-08)

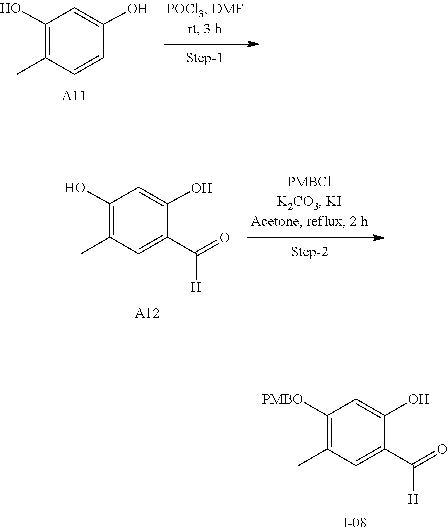

Step-1: Synthesis of 2,4-dihydroxy-5-methylbenzaldehyde (A12): To the stirred solution of 4-methylbenzene-1,3-diol (All) (2 g, 16.1 mmol) and DMF (2.49 mL, 32.2 mmol) in acetonitrile (28 mL) was added POC13 (1.81 mL, 19.3 mmol) dropwise over a period of 15 min at 0° C. The resulting yellow colored solution was further stirred for 3 h at room temperature. The yellow solid obtained was then filtered and washed with cold acetonitrile (3×15 mL). It was then dissolved in hot water (20 mL) and stirred at 50° C. for 30 min. The red colored liquid was then kept for overnight at room temperature and the precipitate obtained was then filtered, washed with water (5 mL×2) and dried to afford desired compound A12 (1 g) as an off-white solid. LCMS (ES) m/z: 153.1 [M+H]⁺.

Step-2: Synthesis of 2-hydroxy-4-[(4-methoxyphenyl)methoxy]-5-methylbenzaldehyde (I-08): Intermediate I-08 (1.2 g) was synthesized by following procedure as described for the synthesis of I-01 (step-1) using 2,4-dihydroxy-5-methylbenzaldehyde (A12) (1.2 g, 7.89 mmol) as the starting material. LCMS (ES) m/z: 273.1[M+H]⁺.

### Intermediate 9: Preparation of 2,4-dihydroxy-5-iodobenzaldehyde (I-09)

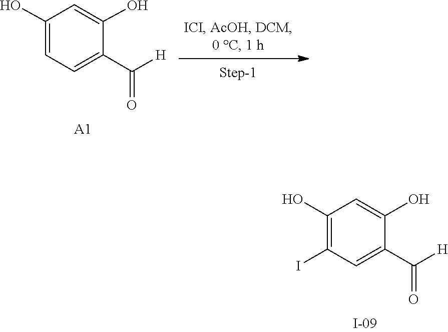 [SMILES: O=Cc1ccc(O)cc1O ; "A1" ; O=Cc1cc(I)c(O)cc1O]

Step-1: Synthesis of 2,4-dihydroxy-5-iodobenzaldehyde (I-09): A solution of ICI (3.22 mL, 61.5 mmol, in 35 mL DCM) was added dropwise to a stirred solution of 2,4-dihydroxybenzaldehyde (A1) (5 g, 36.2 mmol) in acetic acid (25 mL, 437 mmol) at 0° C. The reaction mixture was then allowed to warm to room temperature and the reaction progress was monitored by TLC/LCMS. After complete consumption of starting material (1 h), it was quenched with addition of an aqueous solution of Na₂S₂O₃ (50 mL) and stirred for 15 min. The resulting precipitates were filtered, washed with cold water (10 mL×2) and dried to afford desired compound I-09 (3 g) as a grey solid. LCMS (ES) m/z: 262.87 [M−H]⁺.

### Intermediate 10: Preparation of 2-hydroxy-4-(pyrimidin-2-yloxy)benzaldehyde (I-10)

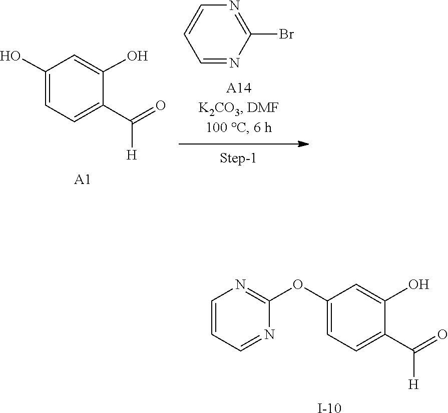

Step-1: Synthesis of 2-hydroxy-4-(pyrimidin-2-yloxy)benzaldehyde (I-10): To a stirred solution of 2,4-dihydroxybenzaldehyde (A1) (1 g, 7.24 mmol) in DMF (30 mL) was added K₂CO₃ (1.1 g, 7.96 mmol) and 2-bromopyrimidine (A14) (1.04 g, 6.52 mmol) under nitrogen atmosphere at room temperature. The reaction mixture was then stirred at 100° C. and the reaction progress was monitored by TLC. After complete consumption of starting material (6 h), it was cooled to room temperature and ice cold water (30 mL) was added to it. Extraction was carried out using EtOAc (2×30 mL); the combined organic extracts were washed with brine solution (2×30 mL), dried over anhydrous Na₂SO₄, filtered and concentrated under reduced pressure. The residue was purified by Combi-Flash (using gradient elution of 0-30% of EtOAc in heptane) to afford the desired compound I-10 (0.4 g) as a pale yellow viscous liquid. LCMS m/z: 217.1 [M+H]⁺.

### Intermediate 11: Preparation of 3-fluoro-2-hydroxy-4-(pyrimidin-2-yloxy)benzaldehyde (I-11)

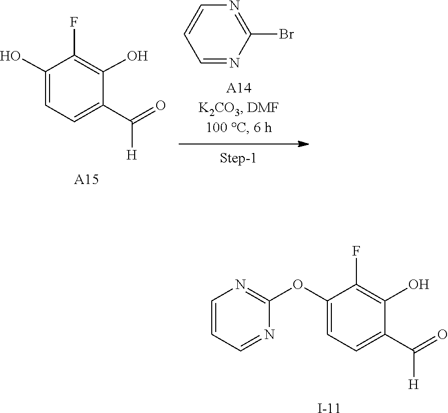

Step-1: Synthesis of 3-fluoro-2-hydroxy-4-(pyrimidin-2-yloxy)benzaldehyde (I-11): Intermediate I-11 (0.130 g) was synthesized by following procedure as described for the synthesis of I-10 (step-1) using 3-fluoro-2,4-dihydroxybenzaldehyde (A15) (650 mg, 4.16 mmol) as the starting material. LCMS (ES) m/z: 235.2 [M+H]⁺.

### Intermediate 12: Preparation of 2-(benzyloxy)-4-(2-oxopyridin-1(2H)-yl)benzaldehyde (1-12)

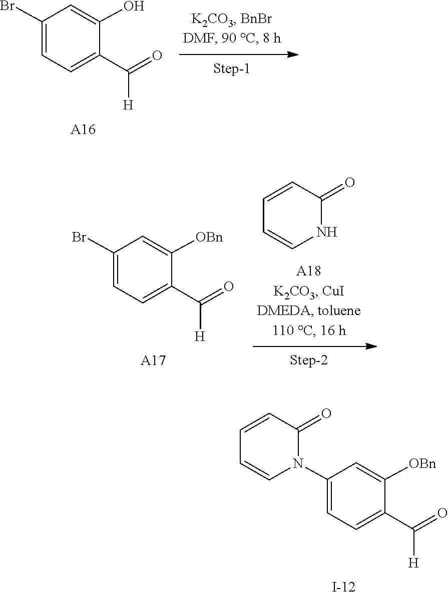

Step-1: Synthesis of 2-(benzyloxy)-4-bromobenzaldehyde (A17): To a stirred solution of 4-bromo-2-hydroxybenzaldehyde (A16) (3 g, 14.9 mmol) in acetonitrile (40 mL) added K₂CO₃ (3.09 g, 22.4 mmol) and benzyl bromide (2.66 mL, 22.4 mmol) at 0° C. The reaction mixture was then stirred at 90° C. while monitoring reaction progress by TLC. After complete consumption of starting material (8 h), the reaction mixture was cooled to room temperature and diluted with water (60 mL). Extraction was carried out using EtOAc (4×50 ml); the combined organic extracts were washed with brine solution (25 mL), dried over anhydrous Na₂SO₄, filtered and concentrated under reduced pressure. The residue was purified by Combi-Flash (using gradient elution of 0-20% of EtOAc in heptane) to afford desired compound A17 (3.1 g) as a colorless solid. LCMS (ES) m/z: 291.0 [M+H]⁺.

Step-2: Synthesis of 2-(benzyloxy)-4-(2-oxo-1,2-dihydropyridin-1-yl)benzaldehyde (I-12): Argon gas was purged through a stirred suspension of 2-(benzyloxy)-4-bromobenzaldehyde (A17) (0.64 g, 2.2 mmol); 1,2-dihydropyridin-2-one (A18) (0.41 g, 4.4 mmol) and K₂CO₃ (0.6 g, 4.4 mmol) in toluene (6 mL) at room temperature for 10 min before addition of DMEDA (0.096 g, 1.1 mmol) and CuI (0.34 g, 1.1 mmol). It was then stirred at 110° C. in a sealed tube. After complete consumption of starting material (16 h), the reaction mixture was cooled to room temperature and water (15 mL) was added to it. Extraction was carried out using EtOAc (2×25 mL); the combined organic extracts were washed with brine solution (2×30 mL), dried over anhydrous Na₂SO₄, filtered and concentrated under reduced pressure The residue was purified by Combi-Flash (using gradient elution of 0-60% of EtOAc in heptane) to afford the desired compound I-12 (0.4 g) as an off-white solid. LCMS (ES) m/z: 306.1 [M+H]⁺.

### Intermediate 13: Preparation of 2-hydroxy-4-(2-oxopyridin-1(2H)-yl)benzaldehyde (I-13)

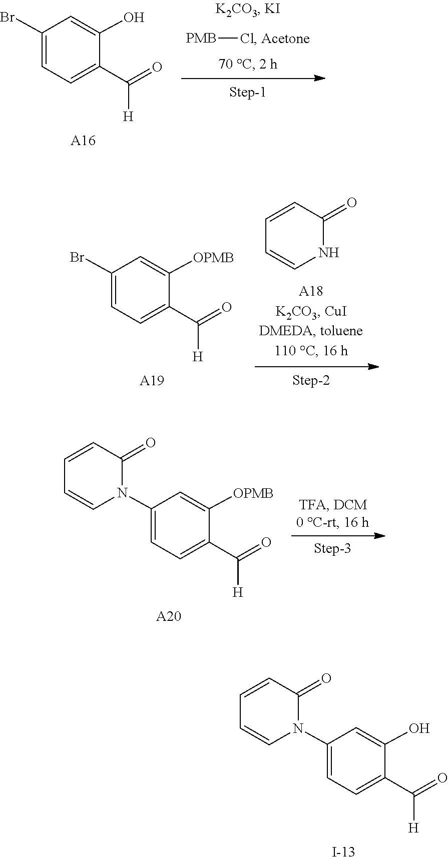

Step-1: Synthesis of 4-bromo-2-[(4-methoxyphenyl) methoxy]benzaldehyde (A19): Compound A19 (1.5 g) was synthesized by following procedure as described for the synthesis of I-11 (step-1) using 4-bromo-2-hydroxybenzaldehyde (A16) (1 g, 4.97 mmol) as the starting material. LCMS (ES) m/z: 321.2 [M+H]⁺.

Step-2: Synthesis of 2-[(4-methoxyphenyl)methoxy]-4-(2-oxo-1,2-dihydropyridin-1-yl)benzaldehyde (A20): Compound A20 (0.6 g) was synthesized by following procedure as described for the synthesis of I-12 (step-2) using 4-bromo-2-[(4-methoxyphenyl)methoxy]benzaldehyde (A19) (0.5 g, 1.56 mmol) as the starting material. LCMS (ES) m/z: 336.2 [M+H]⁺.

Step-3: Synthesis of 2-hydroxy-4-(2-oxo-1,2-dihydropyridin-1-yl)benzaldehyde (I-13): To a stirred solution of 2-[(4-methoxyphenyl)methoxy]-4-(2-oxo-1,2-dihydropyridin-1-yl)benzaldehyde (A20) (0.3 g, 895 μmol) in anhydrous DCM (3 mL) was added trifluoroacetic acid (1.5 mL, 2.68 mmol) at 0° C. and the reaction mixture was then allowed to warm to room temperature. The reaction progress was monitored by TLC/LCMS. After complete consumption of starting material (16 h), volatiles were removed under reduced pressure and water (15 mL) was added to the residue. Extraction was carried out using EtOAc (2×25 mL); the combined organic extracts were washed with saturated NaHCO₃ solution (25 mL), brine solution (20 mL), dried over anhydrous Na₂SO₄, filtered and concentrated under reduced pressure. The residue was purified by Combi-Flash (using gradient elution of 0-10% MeOH in DCM) a to afford desired compound I-13 (0.3 g) as a pale yellow thick oil. LCMS (ES) m/z: 216.1 [M+H]⁺.

### Intermediate 14: Preparation of 2-hydroxy-4-((2-oxopyridin-1(2H)-yl)methyl)benzaldehyde (I-14)

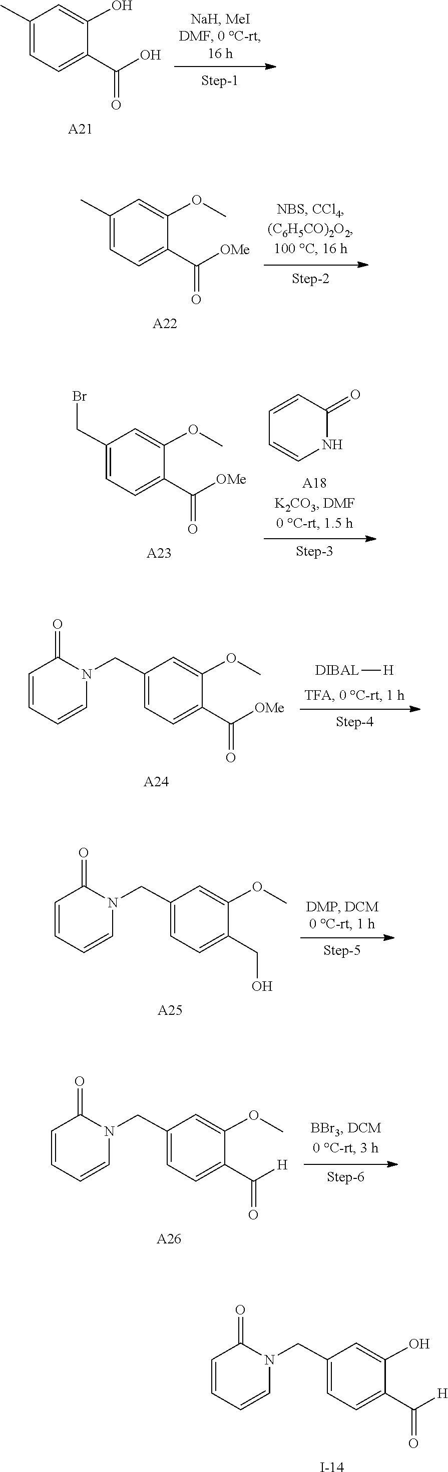

Step-1: Synthesis of methyl 2-methoxy-4-methylbenzoate (A22): To a stirred solution of 2-hydroxy-4-methylbenzoic acid (A21) (5 g, 32.9 mmol) in DMF (50 mL) was added methyl iodide (6.14 mL, 98.6 mmol) at 0° C. followed by portion wise addition of NaH (60% dispersion in mineral oil) (3.94 g, 98.6 mmol). The reaction mixture was then stirred at room temperature for 16 h. The reaction mixture was then quenched with addition of cold water (50 mL) and extraction was carried out using EtOAc (4×50 mL). The combined organic extracts were washed with brine solution (2×50 mL), dried over anhydrous Na₂SO₄, filtered and concentrate under reduced pressure. The residue was purified by Combi-Flash (using gradient elution of 0-30% of EtOAc in heptane) to afford the desired compound A22 (5 g) as a liquid. LCMS m/z: 181.0 [M+H]⁺.

Step-2: Synthesis of methyl 4-(bromomethyl)-2-methoxybenzoate (A23): To a stirred solution of methyl 2-methoxy-4-methylbenzoate (A22) (5 g, 27.7 mmol) in CC14 (129 mL) was added NBS (4.94 g, 27.7 mmol) and benzoyl peroxide (33.6 mg, 139 mol). The reaction mixture was then stirred at 100° C. for 16 h. It was then cooled to room temperature and quenched water (50 mL). Extraction was carried out using DCM (2×50 mL); the combined organic extracts were washed with brine solution (50 mL), dried over Na₂SO₄, filtered and concentrated under reduced pressure. The residue was purified by Combi-Flash (using gradient elution of 0-50% of EtOAc in heptane) to afford desired compound A23 (6 g). LCMS m/z: 259.0 [M+H]⁺.

Step-3: Synthesis of methyl 2-methoxy-4-[(2-oxo-1,2-dihydropyridin-1-yl)methyl]benzoate (A24): To a stirred solution of 1,2-dihydropyridin-2-one (A18) (1.1 g, 11.6 mmol) and methyl 4-(bromomethyl)-2-methoxybenzoate (A23) (3 g, 11.6 mmol) in DMF (32.4 mL) was added K₂CO₃ (4.8 g, 34.7 mmol) at 0° C. and it was then stirred at room temperature. The reaction progress was monitored by TLC/LCMS. After complete consumption of starting material (1.5 h), cold water (20 mL) was added to it and extraction was carried out using EtOAc (2×30 mL). The combined organic extracts were washed with brine solution (50 mL), dried over anhydrous Na₂SO₄. filtered and concentrated under reduced pressure. The residue was purified by Combi-Flash (using gradient elution of 0-60% of EtOAc in heptane) to afford desired compound A24 (1.5 g). LCMS m/z: 273.9 [M+H]⁺.

Step-4: Synthesis of 1-{[4-(hydroxymethyl)-3-methoxyphenyl]methyl}-1,2-dihydropyridin-2-one (A25): To a stirred solution of methyl 2-methoxy-4-[(2-oxo-1,2-dihydropyridin-1-yl)methyl]benzoate (A24) (1.5 g, 5.49 mmol) in THE (15 mL) was added 1 M solution of DIBAL in THE (13.7 mL, 13.7 mmol) at 0° C. and the reaction mixture was allowed to warm to room temperature. The reaction progress was monitored by TLC/LCMS. After complete consumption of starting material (1 h), it was quenched with addition of saturated NH₄Cl solution (20 mL) and extraction was carried out using EtOAc (2×30 mL). The combined organic extracts were washed with brine solution (50 mL), dried over anhydrous Na₂SO₄, filtered and concentrated under reduced pressure. The residue was purified by Combi-Flash (using gradient elution of 0-90% of EtOAc in heptane) to afford desired compound A25 (1.1 g) as an off-white semi-solid. LCMS m/z: 246.1 [M+H]⁺.

Step-5: Synthesis of 2-methoxy-4-[(2-oxo-1,2-dihydropyridin-1-yl)methyl]benzaldehyde (A26): To a stirred solution of 1-f[4-(hydroxymethyl)-3-methoxyphenyl]methyl}-1,2-dihydropyridin-2-one (A25) (1.1 g, 4.48 mmol) in DCM (10 mL) was added DMP (3.8 g, 8.97 mmol) at 0° C., and the reaction mixture was then allowed to warm to room temperature. The reaction progress was monitored by TLC/LCMS. After completion of the reaction (1 h), ice water (30 mL) was added to it and extraction was carried out using EtOAc (2×30 mL). The combined organic extracts were washed with brine solution (2×20 mL), dried over anhydrous Na₂SO₄, filtered and concentrated under reduced pressure. The residue was purified by Combi-Flash (using gradient elution of 0-40% of EtOAc in heptane) to provide desired compound A26 (0.9 g) as an off-white solid. LCMS m/z: 244.1 [M+H]⁺.

Step-6: Synthesis of 2-hydroxy-4-[(2-oxo-1,2-dihydropyridin-1-yl)methyl]benzaldehyde (I-14): To a stirred solution of 2-methoxy-4-[(2-oxo-1,2-dihydropyridin-1-yl)methyl]benzaldehyde (A26) (0.8 g, 3.29 mmol) in anhydrous DCM (8 mL) was added BBr₃ (624 μL, 6.58 mmol) at 0° C. and the reaction mixture was then allowed to warm to room temperature over 1 h. After complete consumption of starting material (1 h), the reaction mixture was quenched with addition of cold water (15 mL) and extraction was carried out using EtOAc (2×30 mL). The combined organic extracts were washed with brine solution, dried over anhydrous Na₂SO₄, filtered and concentrated under reduced pressure. The residue was purified by Combi-Flash (using gradient elution of 0-90% of EtOAc in heptane) to afford the desired I-14 (0.5 g, 2.18 mmol) as a white solid. LCMS m/z: 230.1 [M+H]⁺.

### Intermediate 15: Preparation of 2-hydroxy-4-((2-oxopyrrolidin-1-yl)methyl)benzaldehyde (I-15)

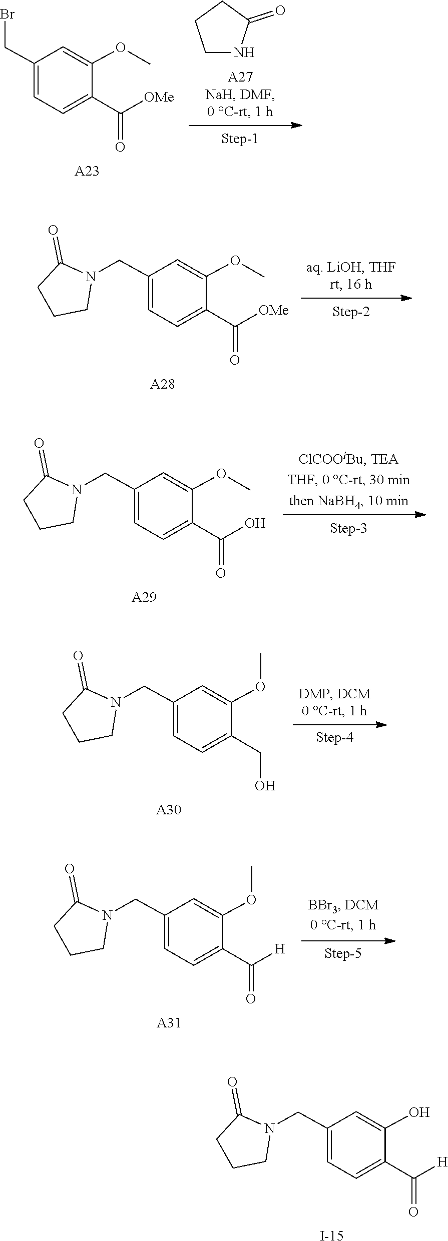

Step-1: Synthesis of methyl 2-methoxy-4-[(2-oxopyrrolidin-1-yl)methyl]benzoate (A28): To a stirred solution of A23 (6 g, 23.2 mmol) in DMF (60 mL, 775 mmol) was added pyrrolidin-2-one (A27) (1.97 g, 23.2 mmol) followed by portion wise addition of NaH (60% dispersion in mineral oil) (1.85 g, 46.3 mmol) at 0° C. The reaction mixture was then allowed to warm to room temperature over 1 h. After complete consumption of starting material (1 h), it was quenched with addition of saturated NH₄Cl solution (50 mL) and extraction was carried out using EtOAc (3×50 mL). The combined organic extracts were washed with brine solution (50 mL×2), dried over anhydrous Na₂SO₄, filtered and concentrated under reduced pressure. The residue was purified by Combi-Flash (using gradient elution of 0-80% of EtOAc in heptane) to afford desired compound A28 (2.9 g) as an off-white solid. LCMS m/z: 264.1 [M+H]⁺.

Step-2: Synthesis of 2-methoxy-4-[(2-oxopyrrolidin-1-yl)methyl]benzoic acid (A29): To a stirred solution of methyl 2-methoxy-4-[(2-oxopyrrolidin-1-yl)methyl]benzoate (A28) (2.6 g, 9.87 mmol) in THE (20 mL) was added an aqueous solution of LiGH (0.71 g, 29.6 mmol, in 6 mL water) and the reaction mixture was stirred at room temperature. After complete consumption of starting material (16 h), it was acidified to pH 6 using 1 N aqueous solution of HCl and extraction was carried out using EtOAc (2×40 mL). The combined organic extracts were washed with brine solution (30 mL), dried over anhydrous Na₂SO₄, filtered and concentrated under reduced pressure to afford desired compound A29 (1.8 g) as an off white solid. LCMS m/z: 250.1 [M+H]⁺.

Step-3: Synthesis of 1-{[4-(hydroxymethyl)-3-methoxyphenyl]methyl}pyrrolidin-2-one (A30): To a stirred solution of 2-methoxy-4-[(2-oxopyrrolidin-1-yl)methyl]benzoic acid (A29) (1.8 g, 7.22 mmol) in THE (18 mL) was added TEA (2.03 mL, 14.4 mmol) and isobutyl chloroformate (1.88 mL, 14.4 mmol) at 0° C. The reaction mixture was allowed to stir at room temperature for 30 min. It was then filtered through a pad of celite and washed with THE (3×10 mL). The filtrate was then cooled to 0° C. and NaBH₄ (1.37 g, 36.1 mmol) in water (0.1 mL) was added to it. The reaction progress was monitored by TLC/LCMS. After complete consumption of starting material (30 min), water (50 mL) was added to it and extraction was carried out using EtOAc (2×40 mL). The combined organic extracts were washed with saturated NaHCO₃ (20 mL), brine solution (20 mL), dried over anhydrous Na₂SO₄, filtered and concentrated under reduced pressure. The residue was purified by Combi-Flash (using gradient elution of 0-30% of EtOAc in heptane) to afford desired compound A30 (1 g) as a yellow gum. LCMS m/z: 236.1 [M+H]⁺.

Step-4: Synthesis of 2-methoxy-4-[(2-oxopyrrolidin-1-yl)methyl]benzaldehyde (A31): Compound A31 (0.9 g) was synthesized by following procedure as described for the synthesis of I-14 (step-5) using 1-{[4-(hydroxymethyl)-3-methoxyphenyl]methyl}pyrrolidin-2-one (A30) (1 g, 4.25 mmol) as the starting material. LCMS (ES) m/z: 234.1 [M+H]⁺.

Step-5: Synthesis of 2-hydroxy-4-[(2-oxopyrrolidin-1-yl)methyl]benzaldehyde (I-15): Intermediate I-15 (0.62 g) was synthesized by following procedure as described for the synthesis of I-14 (step-6) using 2-methoxy-4-[(2-oxopyrrolidin-1-yl)methyl]benzaldehyde (A31) (0.9 g, 3.86 mmol) as the starting material. LCMS (ES) m/z: 220.2 [M+H]⁺.

### Intermediate 16: Preparation of (2,4-dihydroxyphenyl)(²H)formaldehyde (I-16)

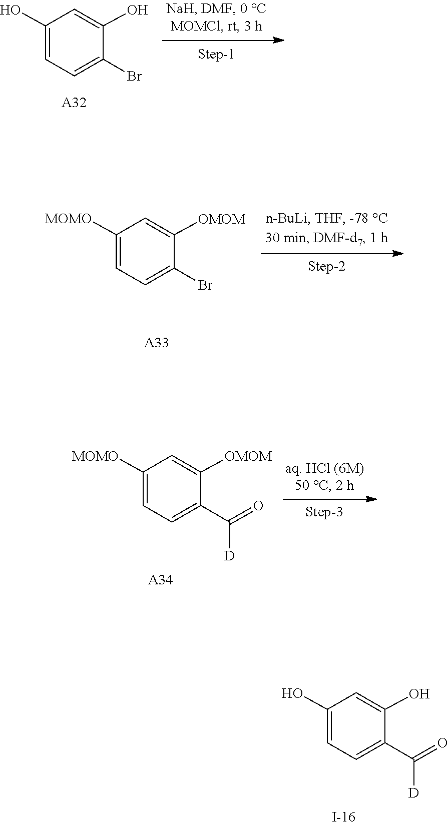

Step-1: Synthesis of 1-bromo-2,4-bis(methoxymethoxy)benzene (A33): To a stirred solution of NaH (381 mg, 15.9 mmol) in DMF (25 mL) was added 4-bromobenzene-1,3-diol (A32) (2.5 g, 13.2 mmol) in DMF at 0° C. and stirred for 15 min, before dropwise addition of MOM-C₁ (5.32 g, 66.1 mmol). After complete consumption of starting material (12 h), the reaction mixture was quenched with addition of cold water (50 mL) and extraction was carried out using EtOAc (2×50 mL). The combined organic extracts were washed with brine solution (2×50 mL), dried over anhydrous Na₂SO₄, filtered and concentrated under reduced pressure. The residue was purified by Combi-Flash (using gradient elution of 0-30% of EtOAc in heptane) to afford the desired compound A33 (1.8 g) as a pale yellow liquid. ¹H NMR (400 MHz, CDCl₃): δ 7.41 (d, J=8.8 Hz, 1H); 6.86 (d, J=2.8 Hz, 1H); 6.64-6.61 (m, 1H); 5.22 (s, 2H); 5.14 (s, 2H); 3.52 (s, 3H); 3.47 (s, 3H).

Step-2: Synthesis of [2,4-bis(methoxymethoxy)phenyl](²H)formaldehyde (A34): To a stirred solution of 1-bromo-2,4-bis(methoxymethoxy)benzene (A33) (5 g, 18 mmol) in anhydrous THE (50 mL) was added n-BuLi (2.5 M in heptane) (10.8 mL, 27.1 mmol) dropwise at −78° C. and the reaction mixture was stirred at 30 min at the same temperature. To this was then added DMF-d₇ (2.81 mL, 36.1 mmol) and the reaction mixture was allowed to warm to room temperature. The reaction progress was monitored by TLC/LCMS. After complete consumption of starting material (1 h), the reaction mixture was quenched with addition of saturated NH₄Cl solution (50 mL) and extraction was carried out using EtOAc (2×50 mL). The combined organic extracts were washed with brine solution (2×25 mL), dried over anhydrous Na₂SO₄, filtered and concentrated under reduced pressure. The residue was purified by Combi-Flash (using gradient elution of 0-35% of EtOAc in heptane) to afford the desired compound A34 (2.4 g, 10.6 mmol) as a pale yellow solid. LCMS m/z: 228.1 [M+H]⁺.

Step-3: Synthesis of (2,4-dihydroxyphenyl)(2H)formaldehyde (I-16): To a stirred solution of [2,4-bis(methoxymethoxy)phenyl](²H)formaldehyde (A34) (2.1 g, 9.24 mmol) in THE (20 mL) was added 6 N HCl solution (10 mL) at 0° C. The reaction mixture was stirred at 50° C. while monitoring reaction progress by TLC. After complete consumption of starting material (2 h), the reaction mixture was cooled to room temperature and diluted with water (50 mL). Extraction was carried out using EtOAc (2×50 mL); the combined organic extracts were washed with brine solution (2×25 mL), dried over anhydrous Na₂SO₄, filtered and concentrated under reduced pressure. The residue was purified by Combi-Flash (using gradient elution of 0-40% of EtOAc in heptane) to afford the desired compound I-16 (0.9 g, 6.47 mmol) as a pale yellow viscous oil. LCMS m/z: 139.9 [M+H]⁺.

### Intermediate 17: Preparation of 3-(4-formyl-3-hydroxyphenyl)-1,1-dimethylurea (1-17)

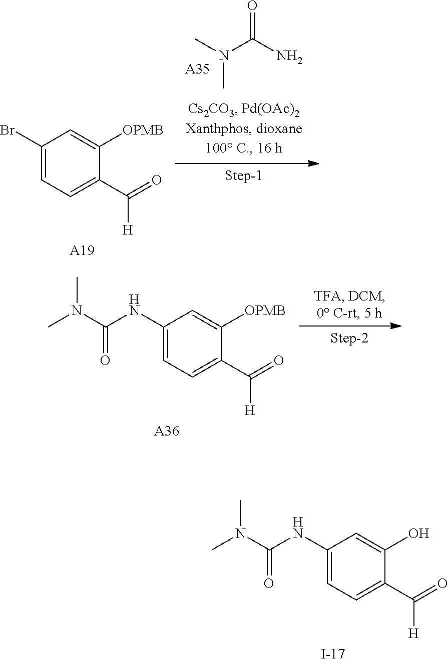

Step-1: Synthesis of 1-{4-formyl-3-[(4-methoxyphenyl)methoxy]phenyl}-3,3-dimethylurea (A36): Argon gas was purged through a stirred suspension of 4-bromo-2-{[4-(methylamino)phenyl]methoxy}benzaldehyde (A19) (1.3 g, 4.06 mmol), Cs₂CO₃ (3.97 g, 12.2 mmol) and 1,1-dimethylurea (A35) (429 mg, 4.87 mmol) in 1,4-dioxane (26 mL) for 10 min, before addition of Pd(OAc)₂ (91.2 mg, 406 μmol) and Xantphos (235 mg, 406 μmol). The reaction mixture was stirred at 100° C. for 16 h. It was then cooled to room temperature and water (30 mL) was added to it. Extraction was carried out using EtOAc (30 mL×3); the combined organic extracts were washed with brine (30 mL), dried over anhydrous Na₂SO₄, filtered and concentrated under reduced pressure. The residue was purified by Combi-Flash (using gradient elution of 0-25% of EtOAc in heptane) to afford the desired compound A36 (950 mg) as an off-white solid. LCMS m/z: 329.2 [M+H]⁺.

Step-2: Synthesis of 1-(4-formyl-3-hydroxyphenyl)-3,3-dimethylurea (I-17): To a stirred solution of 1-{4-formyl-3-[(4-methoxyphenyl)methoxy]phenyl}-3,3-dimethylurea (A36) (0.8 g, 2.44 mmol) in DCM (12 mL) was added TFA (932 μL, 12.2 mmol) at 0° C. under nitrogen atmosphere and the reaction mixture was allowed to warm to room temperature. The reaction progress was monitored by TLC/LCMS. After complete consumption of starting material (5 h), volatiles were removed under reduced pressure and the residue was dissolved in DCM (50 mL). The organic layer was washed with water (2×15 mL), saturated NaHCO₃ solution (2×15 mL), brine solution (2×10 mL), dried over anhydrous Na₂SO₄, filtered and evaporated under reduced pressure. The residue was purified by Combi-Flash (using gradient elution of 0-50% of EtOAc in heptane) to afford the desired compound I-17 (420 mg) as an off-white solid. LCMS m/z: 209.0 [M+H]⁺.

### Intermediate 18: Preparation of 1-(4-formyl-3-hydroxyphenyl)-1,3,3-trimethylurea (I-18)

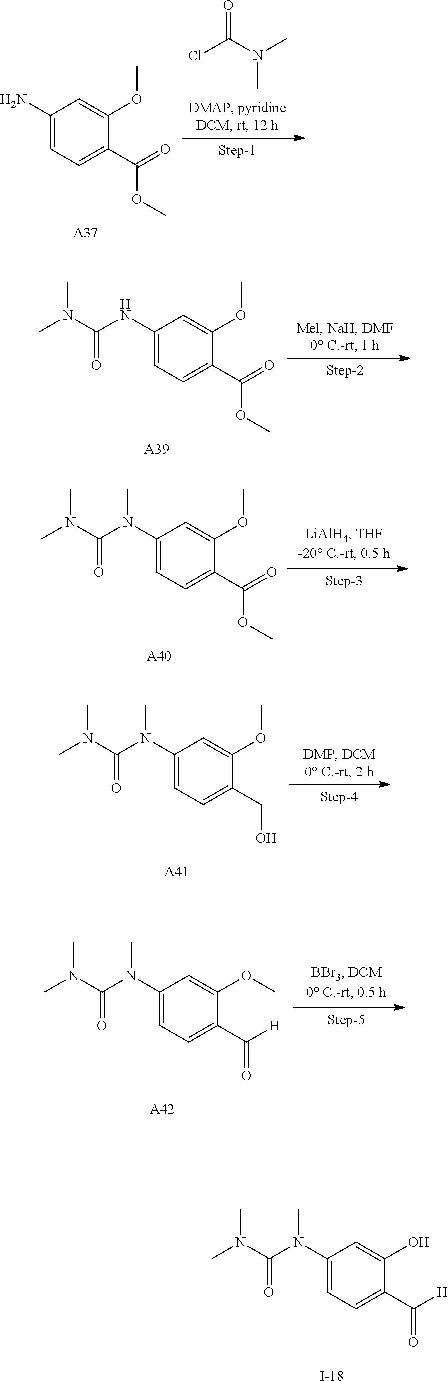

Step-1: Synthesis of methyl 4-[(dimethylcarbamoyl)amino]-2-methoxybenzoate (A39): To a stirred solution of methyl 4-amino-2-methoxybenzoate (A37) (5.0 g, 27.6 mmol) in DCM (20 mL) was added DMAP (3.4 g, 27.6 mmol), pyridine (8.91 mL, 110 mmol) and N,N-dimethylcarbamoyl chloride (5.12 mL, 55.2 mmol). The reaction mixture was stirred at room temperature for 16 h. After completion of the reaction, water (20 mL) was added to it and extraction was carried out with EtOAc (3×50 mL). The combined organic extracts were washed with brine (2×20 mL), dried over anhydrous Na₂SO₄, filtered and concentrated under reduced pressure. The residue was purified by Combi-Flash (using gradient elution of 0-40% of EtOAc in heptane) to afford the desired compound A39 (5.5 g) as an off white solid. LCMS (ES) m/z: 253.37 [M+H]⁺.

Step-2: Synthesis of methyl 4-[(dimethylcarbamoyl)(methyl)amino]-2-methoxybenzoate (A40): To a stirred solution of NaH (60% dispersion in mineral oil) (547 mg, 23.8 mmol) in DMF (30 mL) was added methyl 4-[(dimethylcarbamoyl)amino]-2-methoxybenzoate (A39) (3 g, 11.9 mmol) at 0° C. and stirred for 30 min. To this was then added iodomethane (2.22 mL, 35.7 mmol) and the reaction mixture was stirred at the same temperature, while monitoring reaction progress by TLC. After completion the reaction (0.5 h), the reaction mixture was quenched with water (15 mL) and extraction was carried out with EtOAc (3×40 mL). The combined organic extracts were washed with brine (2×20 mL), dried over anhydrous Na₂SO₄, filtered and concentrated under reduced pressure. The residue was purified by Combi-Flash (using gradient elution of 0-5% of MeOH in DCM) to afford the desired compound A40 (2.8 g) as an off white solid. LCMS (ES) m/z: 267.37 [M+H]⁺.

Step-3: Synthesis of 1-[4-(hydroxymethyl)-3-methoxyphenyl]-1,3,3-trimethylurea (A41): To a stirred solution of methyl 4-[(dimethylcarbamoyl)(methyl)amino]-2-methoxybenzoate (A40) (2.2 g, 8.26 mmol) in anhydrous THE (30 mL) was added lithium aluminum hydride (470 mg, 12.4 mmol) portion wise at −20° C. The reaction was stirred for 15 min at −20° C. and then allowed to warm to room temperature. After completion of the reaction (0.5 h), the reaction mixture was quenched with ice-cold water (10 mL) and extraction was carried out with EtOAc (3×40 mL). The combined organic extracts were washed with brine (2×20 mL), dried over anhydrous Na₂SO₄, filtered and concentrated under reduced pressure to afford desired compound A41 (1.3 g) as a white solid. LCMS (ES) m/z: 239.34 [M+H]⁺.

Step-4: Synthesis of 1-(4-formyl-3-methoxyphenyl)-1,3,3-trimethylurea (A42): To a stirred solution of 1-[4-(hydroxymethyl)-3-methoxyphenyl]-1,3,3-trimethylurea (A41) (1.2 g, 5.04 mmol) in DCM (35 mL) was added DMP (3.84 g, 9.05 mmol) portion wise at 0° C. and the reaction mixture was stirred at room temperature. After completion of the reaction (2 h), ice-cold water (15 mL) was added to it and extraction was carried out using DCM (3×35 mL). The combined organic extracts were washed with brine (2×20 mL), dried over anhydrous Na₂SO₄, filtered and concentrated under reduced pressure. The residue was purified by Combi-Flash (using gradient elution of 0-70% of EtOAc in heptane) to afford the desired compound A42 (0.75 g) as a dark red thick oil. LCMS (ES) m/z: 237.15 [M+H]⁺.

Step-5: Synthesis of 1-(4-formyl-3-hydroxyphenyl)-1,3,3-trimethylurea (I-18): To a stirred solution of 1-(4-formyl-3-methoxyphenyl)-1,3,3-trimethylurea (A42) (650 mg, 2.75 mmol) in DCM (13 mL) was added BBr₃ (0.52 mL, 5.5 mmol) at 0° C. and the reaction mixture was allowed to warm to room temperature. The reaction progress was monitored by TLC/LCMS analysis. After completion of the reaction (0.5 h), the reaction mixture was quenched with ice-cold water (15 mL) and extraction was carried out with EtOAc (3×30 mL). The combined organic extracts were washed with brine (2×20 mL), dried over anhydrous Na₂SO₄, filtered and concentrated under reduced pressure. The residue was purified by Combi-Flash (using gradient elution of 0-30% of EtOAc in heptane) to afford the desired I-18 (0.35 g) as white solid. LCMS (ES) m/z: 223.24 [M+H]⁺.

### Intermediate 19: Preparation of ethyl 2-(4-formyl-3-hydroxyphenyl)acetate (I-19)

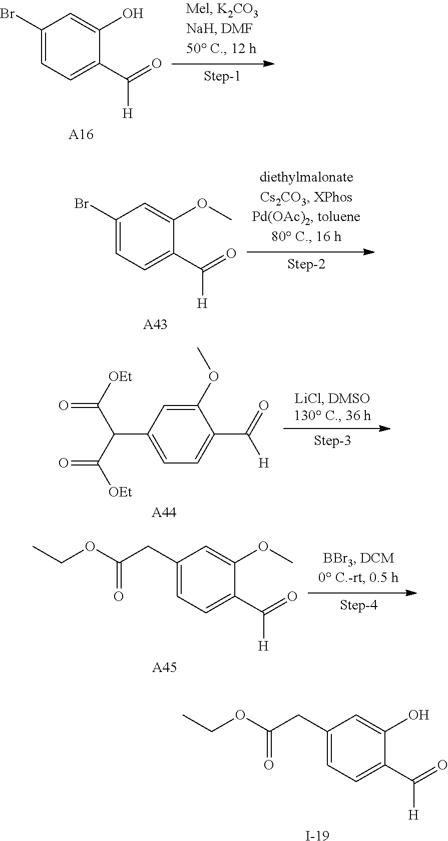

Step-1: Synthesis of 4-bromo-2-methoxybenzaldehyde (A43): To the stirred solution of 4-bromo-2-hydroxybenzaldehyde (A16) (10.0 g, 49.7 mmol) in DMF (90 mL) and was added K₂CO₃ (20.6 g 149 mmol) and iodomethane (9.29 mL, 149 mmol) at room temperature. The reaction mixture was stirred at 50° C., while monitoring reaction progress by TLC. After completion of the reaction (12 h), the reaction mixture was quenched with water (50 mL) and extraction was carried out with EtOAc (3×50 mL). The combined organic extracts were washed with brine (2×20 mL), dried over anhydrous Na₂SO₄, filtered and concentrated under reduced pressure to afford desired compound A43 (10.0 g) as yellow solid. LCMS (ES) m/z: 215 [M+H]⁺.

Step-2: Synthesis of 1,3-diethyl 2-(4-formyl-3-methoxyphenyl)propanedioate (A44): To the stirred solution of 4-bromo-2-methoxybenzaldehyde (A43) (10.0 g, 46.5 mmol) in toluene (200 mL) was added Cs₂CO₃ (30.3 g, 93 mmol) and diethylmalonate (7.09 mL, 46.5 mmol). The reaction mixture was purged with argon for 10 min, before addition of Pd(OAc)₂ (1.04 g, 4.65 mmol) and XPhos (1.11 g, 2.33 mmol). The reaction mixture was then stirred at 80° C. for 16 h. The reaction mixture was then cooled to room temperature and water (100 mL) was added to it. Extraction was carried out using EtOAc (3×70 mL); the combined organic extracts were washed with brine (2×50 mL), dried over anhydrous Na₂SO₄, filtered and concentrated under reduced pressure. The residue was purified by Combi-Flash (using gradient elution of 0-15% of EtOAc in heptane) to afford the desired compound A44 (9 g) as a white solid. LCMS (ES) m/z: 293.30 [M−H]⁺.

Step-3: Synthesis of ethyl 2-(4-formyl-3-methoxyphenyl)acetate (A45): To a stirred solution of 1,3-diethyl 2-(4-formyl-3-methoxyphenyl)propanedioate (A44) (8.5 g, 28.9 mmol) in DMSO (35 mL) was added lithium chloride (3.67 g, 86.6 mmol) and the reaction mixture was stirred at 130° C., while monitoring reaction progress by TLC. After completion of the reaction (36 h), it was cooled to room temperature and water (50 mL) was added to it. Extraction was carried out using EtOAc (3×60 mL); the combined organic extracts were washed with brine (2×50 mL), dried over anhydrous Na₂SO₄, filtered and concentrated under reduced pressure to afford desired compound A45 (1.2 g) as a white solid. LCMS (ES) m/z: 221.11 [M−H]⁺.

Step-4: Synthesis of ethyl 2-(4-formyl-3-hydroxyphenyl)acetate (I-19): Intermediate I-19 (1.65 g) was synthesized by following procedure as described for the synthesis of 1-18 (step-5) using ethyl 2-(4-formyl-3-methoxyphenyl)acetate (A45) (2.5 g, 11.2 mmol) as the starting material. LCMS (ES) m/z: 209.26 [M+H]⁺.

### Intermediate 20: Preparation of 5-chloro-2-hydroxy-4-iodobenzaldehyde (I-20)

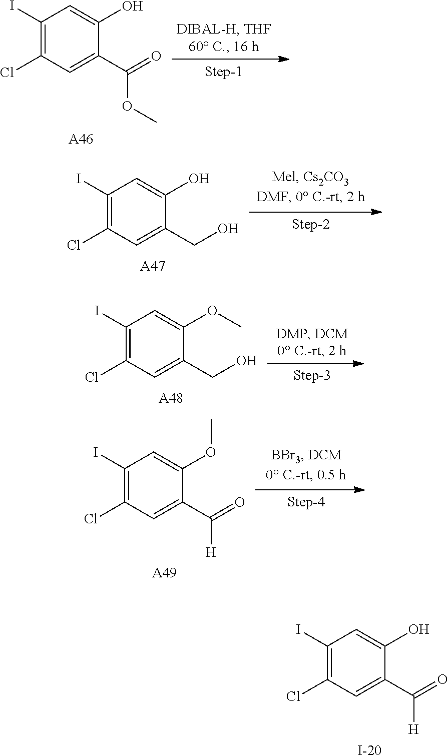

Step-1: Synthesis of 4-chloro-2-(hydroxymethyl)-5-iodophenol (A47): To stirred the solutions of methyl 5-chloro-2-hydroxy-4-iodobenzoate (A46) (6.0 g, 19.2 mmol) in THE (60 mL) was added a 1 M solution of DIBAL (in toluene) (57.6 mL, 57.6 mmol) at 0° C. The reaction mixture was then stirred at 60° C., while monitoring reaction progress by TLC/LCMS. After completion of the reaction (16 h), the reaction mixture was cooled to 0° C. and quenched with slow addition of aqueous 1 N HCl solution (20 mL). Extraction was carried out using EtOAc (3×70 mL); the combined organic extracts were washed with brine (2×30 mL), dried over anhydrous Na₂SO₄, filtered and concentrated under reduced pressure. The residue was purified by Combi-Flash (using gradient elution of 0-60% of EtOAc in heptane) to afford desired compound A47 (5.8 g) as an off white solid. LCMS (ES) m/z: 284.90 [M−H]⁺.

Step-2: Synthesis of (5-chloro-4-iodo-2-methoxyphenyl)methanol (A48): To a stirred solution of 4-chloro-2-(hydroxymethyl)-5-iodophenol (A47) (5.8 g, 20.4 mmol) in DMF (25 mL) were added Cs₂CO₃ (6.64 g, 20.4 mmol) and iodomethane (1.27 mL, 20.4 mmol) and the reaction mixture was stirred at room temperature for 2 h. After completion, water (30 mL) was added to it and the precipitated solid was filtered, washed with ice cold water (3×40 mL) and dried to afford desired compound A48 (5.7 g) as a white solid. ¹H NMR (400 MHz, DMSO-d₆): δ 7.44 (s, 1H); 7.40 (s, 1H); 5.23 (t, J=5.6 Hz, 1H); 4.40 (d, J=5.6 Hz, 2H); 3.78 (s, 3H).

Step-3: Synthesis of 5-chloro-4-iodo-2-methoxybenzaldehyde (A49): Compound A49 (4.1 g) was synthesized by following procedure as described for the synthesis of I-18 (step-4) using (5-chloro-4-iodo-2-methoxyphenyl)methanol (A48) (5.7 g, 8.38 mmol) as the starting material. ¹H NMR (400 MHz, DMSO-d₆): δ 10.22 (s, 1H); 7.81 (s, 1H); 7.68 (s, 1H); 3.32 (s, 3H).

Step-4: Synthesis of 5-chloro-2-hydroxy-4-iodobenzaldehyde (I-20): Intermediate (I-20) (3.6 g) was synthesized by following procedure as described for the synthesis of I-18 (step-5) using 5-chloro-4-iodo-2-methoxybenzaldehyde A49 (4.1 g, 13.8 mmol) as the starting material. ¹H NMR (400 MHz, DMSO-d₆): δ 11.14 (s, 1H); 10.18 (s, 1H); 7.66 (s, 1H); 7.59 (s, 1H).

### Intermediate 21: Preparation of (2,4-bis(benzyloxy)phenyl)methanamine (I-21)

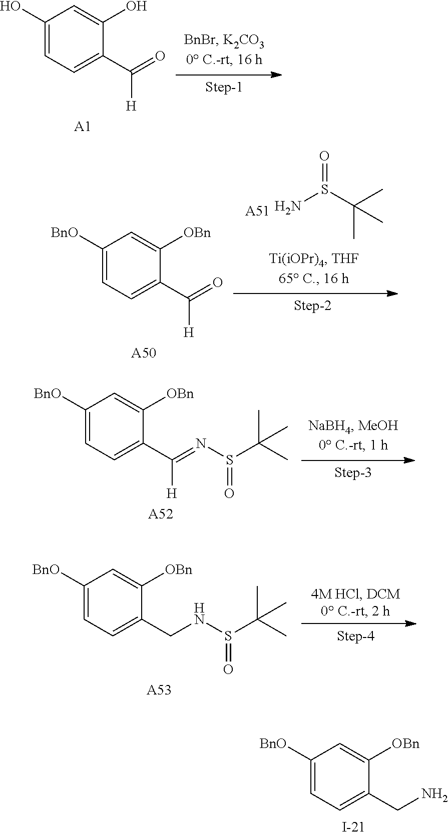

Step-1: Synthesis of 2,4-bis(benzyloxy)benzaldehyde (A50): To a stirred solution of 2,4-dihydroxybenzaldehyde (A1) (3 g, 21.7 mmol) in DMF (30 mL) was added K₂CO₃ (6 g, 43.4 mmol) and benzyl bromide (5.16 mL, 43.4 mmol); and the reaction mixture was stirred at room temperature for 16 h. Ice water (150 mL) was then added to it and extraction was carried out using EtOAc (3×50 mL). The combined organic extracts were washed with saturated sodium bicarbonate solution (30 mL), brine solution (30 mL), dried over anhydrous Na₂SO₄, filtered and concentrated under reduced pressure. The residue was purified by Combi-Flash (using gradient elution of 0-30% of EtOAc in heptane) to afford the desired compound A50 (6.5 g) as an off white solid. LCMS m/z: 319.1 [M+H]⁺.

Step-2: Synthesis of (E)-N-(2,4-bis(benzyloxy)benzylidene)-2-methylpropane-2-sulfinamide (A52): To a stirred solution of 2,4-bis(benzyloxy)benzaldehyde (A50) (4.0 g, 12.6 mmol) in THE (40 mL) was added 2-methylpropane-2-sulfinamide (A51) (1.52 g, 12.6 mmol) and Ti(OⁱPr)₄ (4.46 mL, 15.1 mmol) at room temperature. The reaction mixture was allowed to stir at 65° C. and the reaction progress was monitored by TLC/LCMS. After complete consumption of starting material (16 h), it was cooled to room temperature and saturated NaHCO₃ solution (30 mL) was added to it. Extraction was carried out using EtOAc (2×40 mL); the combined organic extracts were washed with brine solution (30 mL), dried over anhydrous Na₂SO₄, filtered and concentrated under reduced pressure. The residue was purified by Combi-Flash (using gradient elution of 0-35% of EtOAc in heptane) to afford the desired compound A52 (4.4 g) as an off-white solid. LCMS m/z found 422.1 [M+H]⁺.

Step-3: Synthesis of N-(2,4-bis(benzyloxy)benzyl)-2-methylpropane-2-sulfinamide (A53): To a stirred suspension of A52 (4.4 g, 10.4 mmol) in MeOH (20 mL) was added NaBH₄ (0.79 g, 20.9 mmol) at 0° C. and the reaction mixture was allowed to stir for 30 min at room temperature. The reaction progress was monitored by TLC/LCMS. After complete consumption of starting material (30 min), the reaction mixture was quenched with addition of water (50 mL) and extraction was carried out using EtOAc (2×40 mL). The combined organic extracts were washed with brine solution (30 mL), dried over anhydrous Na₂SO₄, filtered and concentrated under reduced pressure. The residue was purified by Combi-Flash (using gradient elution of 0-50% of EtOAc in heptane) to afford the desired compound A53 (4.3 g) as an off white solid. LCMS m/z: 424.1 [M+H]⁺.

Step-4: Synthesis of (2,4-bis(benzyloxy)phenyl)methanamine (I-21): To a stirred solution of N-{[2,4-bis(benzyloxy)phenyl]methyl}-2-methylpropane-2-sulfinamide (A53) (4.3 g, 10.2 mmol) in DCM (4 mL) was added a 4 M solution of HCl in dioxane (5.08 mL, 20.3 mmol) at 0° C. and the reaction mixture was allowed to stir at room temperature. The reaction progress was monitored by TLC/LCMS. After complete consumption of starting material (2 h), volatiles were removed under reduced pressure and saturated NaHCO₃ solution (20 mL) was added to the residue. Extraction was carried out using DCM (30 mL×3); the combined organic extracts were washed with brine (30 mL), dried over anhydrous Na₂SO₄, filtered and concentrated under reduced pressure. The crude was washed with diethyl ether (2×10 mL) and dried to afford the desired compound I-21 (2.2 g) as an off white solid. ¹H NMR (400 MHz, DMSO-d₆): δ 7.45-7.30 (m, 12H); 7.19 (d, J=8.0 Hz, 1H); 6.70 (d, J=2.0 Hz, 1H); 6.56-6.54 (m, 1H); 5.08 (d, J=15.2 Hz, 4H); 3.63 (s, 2H).

### Intermediate 22: Preparation of 1-{2-hydroxy-4-[(4-methoxyphenyl)methoxy]phenyl}ethan-1-one (I-22)

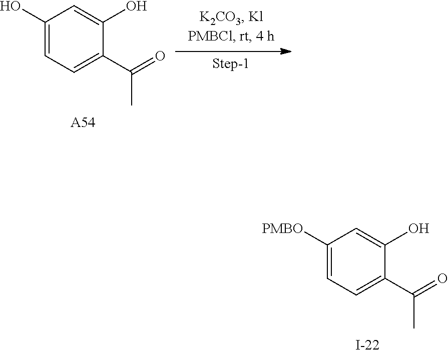

Step-1: Synthesis of 1-{2-hydroxy-4-[(4-methoxyphenyl)methoxy]phenyl}ethan-1-one (I-22): Intermediate I-22 (4.0 g) was synthesized by following procedure as described for the synthesis of I-01 (step-1) using 1-(2,4-dihydroxyphenyl)ethan-1-one (A54) (2.5 g, 16.4 mmol) as the starting material. LCMS (ES) m/z: 273.1 [M+H]⁺.

### Intermediate 23: Preparation of 1-(3-fluoro-2-hydroxy-4-((4-methoxybenzyl)oxy)phenyl)ethan-1-one (I-23)

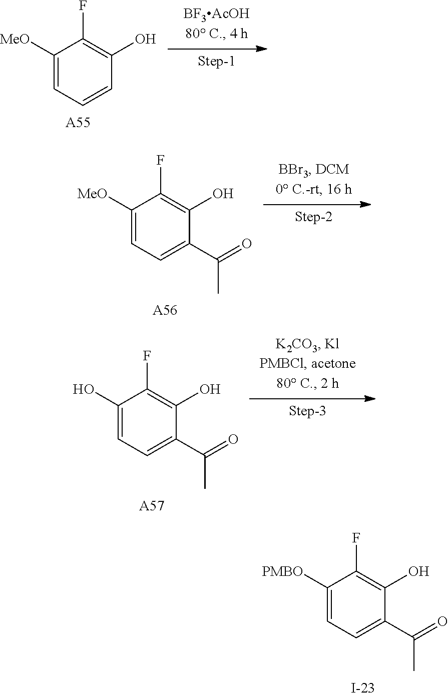

Step-1: Synthesis of 1-(3-fluoro-2-hydroxy-4-methoxyphenyl)ethan-1-one (A56): A stirred solution of 2-fluoro-3-methoxyphenol (A55) (3 g, 21.1 mmol) in BF3AcOH (12 mL) was slowly heated to 80° C. and the reaction progress was monitored by TLC/LCMS. After complete consumption of starting material (4 h), the reaction mixture was cooled to room temperature and quenched with addition of water (50 mL). Extraction was carried out using EtOAc (2×50 mL); the combined organic extracts were washed with brine solution (50 mL), dried over anhydrous Na₂SO₄, filtered and concentrated under reduced pressure The residue was purified by Combi-Flash (using gradient elution of 0-50% of EtOAc in heptane) to afford the desired compound A56 (3 g) as an off-white solid. LCMS (ES) m/z: 185.0 [M+H]⁺.

Step-2: Synthesis of 1-(3-fluoro-2,4-dihydroxyphenyl)ethan-1-one (A57): To a stirred solution of 1-(3-fluoro-2-hydroxy-4-methoxyphenyl)ethan-1-one (A56) (3 g, 16.3 mmol) in DCM (30 mL) was added BBr₃ (3.14 mL, 32.6 mmol) dropwise at 0° C. and the reaction mixture was allowed to warm to room temperature over 2 h. The reaction progress was monitored by TLC/LCMS. After complete consumption of starting material (16 h), the reaction mixture was quenched with addition of cold water (50 mL) and extraction was carried out using EtOAc (3×50 mL). The combined organic extracts were washed with brine solution (50 mL), dried over anhydrous Na₂SO₄, filtered and concentrated under reduced pressure. The residue was purified by Combi-Flash (using gradient elution of 0-50% of EtOAc in heptane) to afford the desired compound A57 (2 g) as a colorless solid. LCMS (ES) m/z: 171.1 [M+H]⁺.

Step-3: Synthesis of 1-{3-fluoro-2-hydroxy-4-[(4-methoxyphenyl)methoxy]phenyl}ethan-1-one (I-23): Intermediate I-23 (2.0 g) was synthesized by following procedure as described for the synthesis of I-01 (step-1) using 1-(3-fluoro-2,4-dihydroxyphenyl)ethan-1-one (A57) (2 g, 11.8 mmol) as the starting material. LCMS (ES) m/z: 291.0 [M+H]⁺.

### Intermediate 24: Preparation of 1-(5-fluoro-2-hydroxy-4-((4-methoxybenzyl)oxy)phenyl)ethan-1-one (I-24)

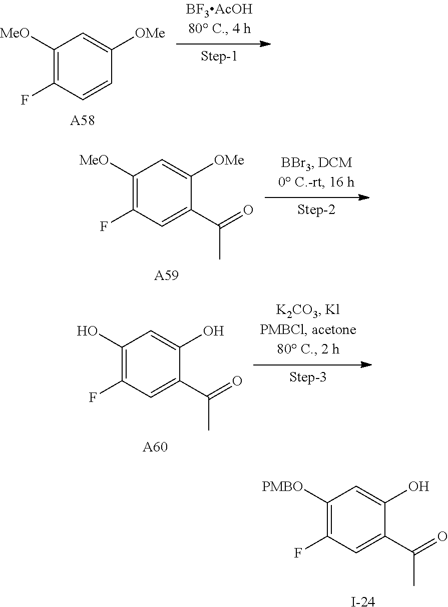

Step-1: Synthesis of 1-(5-fluoro-2,4-dimethoxyphenyl)ethan-1-one (A59): Compound A59 (3.8 g) was synthesized by following procedure as described for the synthesis of I-23 (step-1) using 1-fluoro-2,4-dimethoxybenzene (A58) (2 g, 12.8 mmol) as the starting material. LCMS (ES) m/z: 199.0 [M+H]⁺.

Step 2: Synthesis of 1-(5-fluoro-2,4-dihydroxyphenyl)ethan-1-one (A60): Compound A60 (3.2 g) was synthesized by following procedure as described for the synthesis of I-23 (step-2) using 1-(5-fluoro-2,4-dimethoxyphenyl)ethan-1-one (A59) (3.8 g, 19.2 mmol) as the starting material. LCMS (ES) m/z: 171 [M+H]⁺.

Step 3: Synthesis of 1-f5-fluoro-2-hydroxy-4-[(4-methoxyphenyl)methoxy]phenyl}ethan-1-one (I-24): Intermediate I-24 (1.5 g) was synthesized by following procedure as described for the synthesis of I-01 (step-1) using 1-(2-fluoro-4,6-dihydroxyphenyl)ethan-1-one (A60) (1.5 g, 8.82 mmol) as the starting material. LCMS (ES) m/z: 290.9 [M+H]⁺.

### Intermediate 25: Preparation of 1-(2-fluoro-6-hydroxy-4-((4-methoxybenzyl)oxy)phenyl)ethan-1-one (1-25)

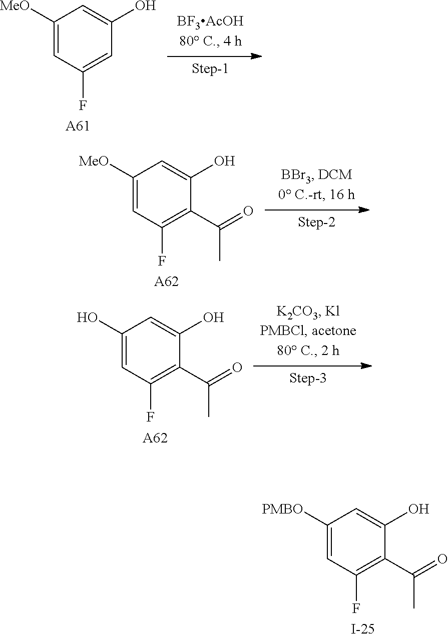

Step 1: Synthesis of 1-(2-fluoro-6-hydroxy-4-methoxyphenyl)ethan-1-one (A62): Compound A62 (1.8 g) was synthesized by following procedure as described for the synthesis of 1-23 (step-1) using 1-fluoro-3,5-dimethoxybenzene (A61) (2 g, 12.8 mmol) as the starting material. LCMS (ES) m/z: 184.9 [M+H]⁺.

Step 2: Synthesis of 1-(2-fluoro-4,6-dihydroxyphenyl)ethan-1-one (A63): Compound A63 (1.5 g) was synthesized by following procedure as described for the synthesis of I-23 (step-2) using 1-(2-fluoro-6-hydroxy-4-methoxyphenyl)ethan-1-one (A62) (1.8 g, 9.77 mmol) as the starting material. LCMS (ES) m/z: 171.1 [M+H]⁺.

Step 3: Synthesis of 1-{2-fluoro-6-hydroxy-4-[(4-methoxyphenyl)methoxy]phenyl}ethan-1-one (I-25): Intermediate 1-25 (2.3 g) was synthesized by following procedure as described for the synthesis of 1-01 (step-1) using 1-(2-fluoro-4,6-dihydroxyphenyl)ethan-1-one (A63) (1.5 g, 8.82 mmol) as the starting material. LCMS (ES) m/z: 290.9 [M+H]⁺.

### Intermediate 26: Preparation of 3-(2,4-bis(benzyloxy)phenyl)oxetan-3-amine (I-26)

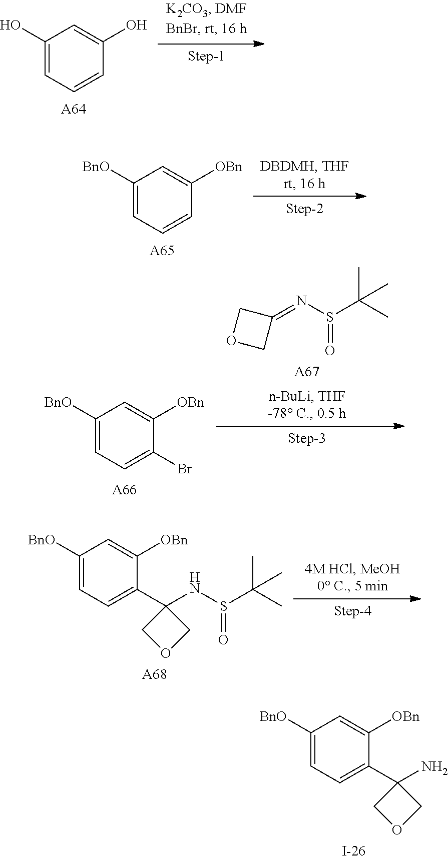

Step-1: Synthesis of 1,3-bis(benzyloxy)benzene (A65): To a stirred solution of benzene-1,3-diol (A64) (10 g, 90.8 mmol) in DMF (100 mL) was added K₂CO₃ (37.7 g, 272 mmol) and (bromomethyl)benzene (27.5 mL, 227 mmol); and the reaction mixture was stirred room temperature for 16 h. Water (100 mL) was then added to it and extraction was carried out using EtOAc (3×100 mL). The combined organic extracts were washed with brine solution (100 mL×3), dried over anhydrous Na₂SO₄, filtered and concentrated under reduced pressure. The residue was purified by Combi-Flash (using gradient elution of 0-30% of EtOAc in heptane) to afford the desired compound A65 (18 g) as a colorless solid. LCMS m/z: 291.2 [M+H]⁺.

Step-2: Synthesis of 2,4-bis(benzyloxy)-1-bromobenzene (A66): To a stirred solution of 1,3-bis(benzyloxy)benzene (A65) (19.3 g, 66.5 mmol) in THE (400 mL) was added 1,3-dibromo-5,5-dimethylimidazolidine-2,4-dione (9.5 g, 33.2 mmol) at 0° C. and the reaction mixture as allowed to warm to room temperature over 30 min. The reaction progress was monitored by TLC/LCMS. After complete consumption of starting material (16 h), the reaction mixture was quenched with water (100 mL) and extraction was carried out using EtOAc (3×200 mL). The combined organic extracts were washed with brine solution (3×100 mL), dried over anhydrous Na₂SO₄, filtered and concentrated under reduced pressure. The residue was purified by Combi-Flash (using gradient elution of 0-30% of EtOAc in heptane) to afford the desired A66 (24 g) as a pale yellow solid. LCMS m/z: 369.1 [M+H]⁺.

Step-3: Synthesis of N-{3-[2,4-bis(benzyloxy)phenyl]oxetan-3-yl}-2-methylpropane-2-sulfinamide (A68): To a stirred solution of 2,4-bis(benzyloxy)-1-bromobenzene (A66) (12 g, 32.5 mmol) in anhydrous THE (120 mL) was added n-BuLi (2.5 M in hexane) (14.3 mL, 35.7 mmol) at −78° C. and the reaction was stirred for 10 min. To this was then added 2-methyl-N-(oxetan-3-ylidene)propane-2-sulfinamide (A67) (5.7 g, 32.5 mmol, in 10 mL THF) and stirred at −78° C. for additional 30 min. The reaction mixture was quenched with addition of saturated NH₄Cl solution (100 mL) and extraction was carried out using EtOAc (3×70 mL). The combined organic extracts were washed with brine solution (3×70 mL), dried over Na₂SO₄ and evaporated under reduced pressure. The residue was purified by Combi-Flash (using gradient elution of 0-50% of EtOAc in heptane) to afford the desired compound A68 (4 g) as a pale yellow solid. LCMS m/z: 466.2 |M+H|⁺.

Step-4: Synthesis of 3-[2,4-bis(benzyloxy)phenyl]oxetan-3-amine (I-26): To a stirred solution of N-{3-[2,4-bis(benzyloxy)phenyl]oxetan-3-yl}-2-methylpropane-2-sulfinamide (A68) (2 g, 4.3 mmol) in MeOH (20 mL) was added a 4 M solution of HCl in 1,4-dioxane (5.3 mL, 21.5 mmol) at 0° C. The reaction progress was monitored by TLC/LCMS. After complete consumption of starting material (5 min), the reaction mixture was quenched with addition of a saturated NaHCO₃ solution (20 mL). Extraction was carried out using EtOAc (3×40 mL); the combined organic extracts were washed with brine solution (3×20 mL), dried over anhydrous Na₂SO₄, filtered and concentrated under reduced pressure. The residue was purified by Combi-Flash (using gradient elution of 0-50% of EtOAc in heptane) to afford the desired compound I-26 (1.5 g) as a pale yellow solid. LCMS m/z: 360.1 [M−H]⁺.

### Intermediate 27: Preparation of 3-(2,4-bis(benzyloxy)-3-fluorophenyl)oxetan-3-amine (I-27)

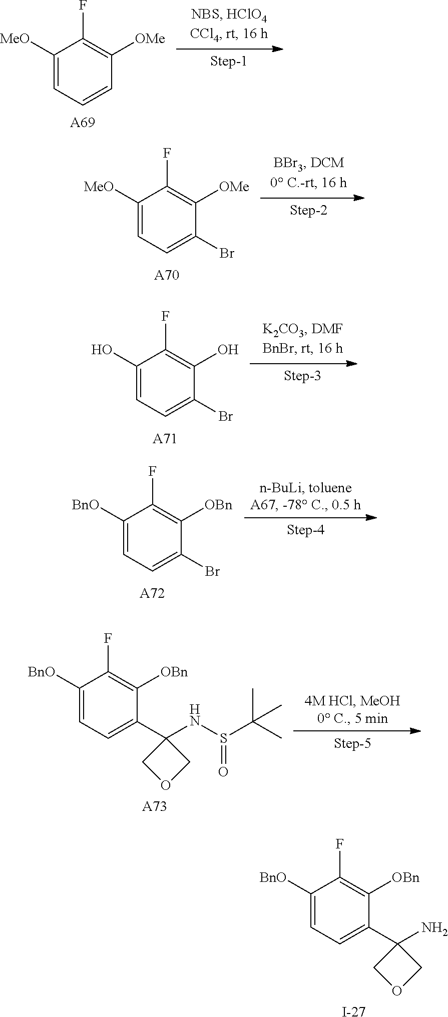

Step-1: Synthesis of 1-bromo-3-fluoro-2,4-dimethoxybenzene (A70): To a stirred solution of 2-fluoro-1,3-dimethoxybenzene (A69) (5 g, 32 mmol) in CCl₄ (25 mL) was added NBS (6.27 g, 35.2 mmol) and 60% of HClO₄ (134 mg, 0.8 mmol) at 0° C. The reaction mixture was stirred at room temperature, while monitoring reaction progress by TLC/LCMS. After complete consumption of starting material (3 h), the reaction mixture was filtered and the filtrate was concentrated under reduced pressure. The residue was purified by Combi-Flash (using gradient elution of 0-10% of EtOAc in heptane) to afford desired compound A70 (7.0 g) as a pale yellow oil. ¹H NMR (400 MHz, DMSO-d₆): δ 7.34 (dd, J=9.2 Hz, 2.0 Hz, 1H); 6.89 (t, J=8.8 Hz, 1H); 3.83 (d, J=7.6 Hz, 6H).

Step-2: Synthesis of 4-bromo-2-fluorobenzene-1,3-diol (A71): Compound A71 (7.6 g) was synthesized by following procedure as described for the synthesis of I-03 (step-2) using 1-bromo-3-fluoro-2,4-dimethoxybenzene (A70) (10.0 g, 42.5 mmol) as the starting material. LCMS (ES) m/z: 207.0 [M+H]⁺.

Step-3: Synthesis of 1,3-bis(benzyloxy)-4-bromo-2-fluorobenzene (A72): Compound A72 (18 g) was synthesized by following procedure as described for the synthesis of I-26 (step-1) using 4-bromo-2-fluorobenzene-1,3-diol (A71) (11.8 g, 57 mmol) as the starting material. LCMS (ES) m/z: 386.9 [M+H]⁺.

Step-4: Synthesis of N-{3-[2,4-bis(benzyloxy)-3-fluorophenyl]oxetan-3-yl}-2-methylpropane-2-sulfinamide (A73): Compound A73 (1.4 g) was synthesized by following procedure as described for the synthesis of I-26 (step-3) using 1,3-bis(benzyloxy)-4-bromo-2-fluorobenzene (A72) (5.59 g, 14.4 mmol) as the starting material (toluene was used as the solvent). LCMS (ES) m/z: 484.2 [M+H]⁺.

Step-5: Synthesis of 3-[2,4-bis(benzyloxy)-3-fluorophenyl]oxetan-3-amine (I-27): Intermediate I-27 (1.7 g) was synthesized by following procedure as described for the synthesis of I-26 (step-4) using N-{3-[2,4-bis(benzyloxy)-3-fluorophenyl]oxetan-3-yl1-2-methyl propane-2-sulfinamide (A73) (2.9 g, 6 mmol) as the starting material. ¹H NMR (400 MHz, DMSO-d₆): δ 7.47-7.33 (m, 10H); 6.96-6.88 (m, 2H); 5.18 (d, J=11.6 Hz, 4H); 4.85 (d, J=6 Hz, 2H); 4.40 (d, J=6 Hz, 2H).

### Intermediate 28: Preparation of 3-(2,4-bis(benzyloxy)-5-fluorophenyl)oxetan-3-amine (I-28)

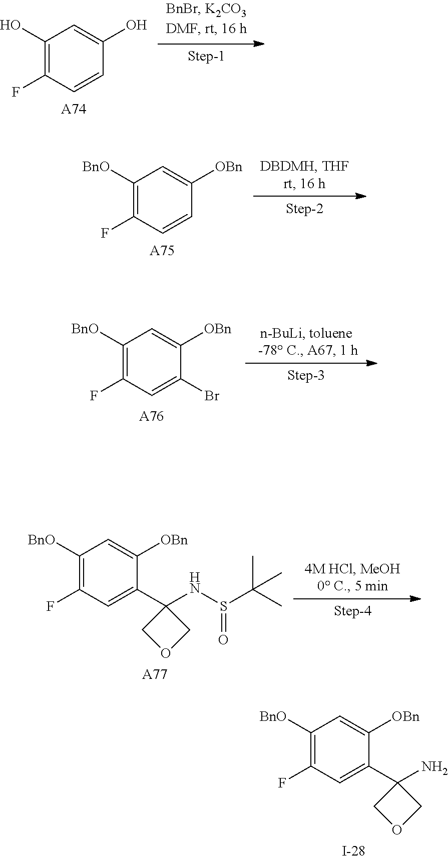

Step-1: Synthesis of 2,4-bis(benzyloxy)-1-fluorobenzene (A75): Compound A75 (18 g) was synthesized by following procedure as described for the synthesis of I-26 (step-1) using 4-fluorobenzene-1,3-diol (A74) (4 g, 31.2 mmol) as the starting material. LCMS (ES) m/z: 309.1 [M+H]⁺.

Step-2: Synthesis of 1,5-bis(benzyloxy)-2-bromo-4-fluorobenzene (A76): Compound A76 (1.8 g) was synthesized by following procedure as described for the synthesis of I-26 (step-2) using 2,4-bis(benzyloxy)-1-fluorobenzene (A75) (2 g, 6.49 mmol) as the starting material. LCMS (ES) m/z: 387.0 [M+H]⁺.

Step-3: Synthesis of N-{3-[2,4-bis(benzyloxy)-5-fluorophenyl]oxetan-3-yl}-2-methylpropane-2-sulfinamide (A77): Compound A77 (1.1 g) was synthesized by following procedure as described for the synthesis of I-26 (step-3) using 1,5-bis(benzyloxy)-2-bromo-4-fluorobenzene (A76) (7.29 g, 18.8 mmol) as the starting material (toluene was used as the solvent). LCMS (ES) m/z: 484.2 [M+H]⁺.

Step-4: Synthesis of 3-[2,4-bis(benzyloxy)-5-fluorophenyl]oxetan-3-amine (I-28): Intermediate I-28 (960 mg) was synthesized by following procedure as described for the synthesis of I-26 (step-4) using N-{3-[2,4-bis(benzyloxy)-5-fluorophenyl]oxetan-3-yl}-2-methylpropane-2-sulfinamide (A77) (1.6 g, 3.31 mmol) as the starting material. LCMS m/z: 363.1 [M−18H]⁺.

### Intermediate 29: Preparation of 3-(2,4-bis(benzyloxy)-6-fluorophenyl)oxetan-3-amine (I-29)

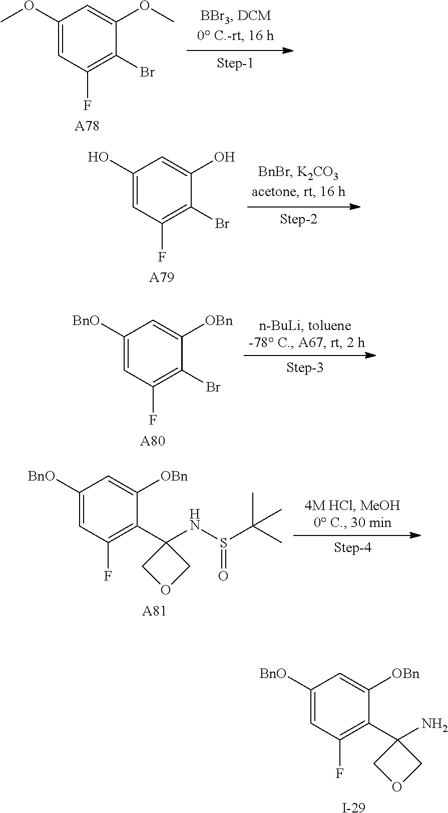

Step-1: Synthesis of 4-bromo-5-fluorobenzene-1,3-diol (A79): Compound A79 (3.5 g) was synthesized by following procedure as described for the synthesis of I-03 (step-2) using 2-bromo-1-fluoro-3,5-dimethoxybenzene (A78) (4.5 g, 19.1 mmol) as the starting material. LCMS (ES) m/z: 206.9 [M+H]⁺.

Step-2: Synthesis of 1,5-bis(benzyloxy)-2-bromo-3-fluorobenzene (A80): Compound A80 (6.8 g) was synthesized by following procedure as described for the synthesis of I-26 (step-1) using 4-bromo-5-fluorobenzene-1,3-diol (A79) (4 g, 19.3 mmol) as the starting material. ¹H NMR (400 MHz, CDCl₃): δ 7.47-7.41 (m, 2H); 7.39-7.30 (m, 10H); 5.16 (s, 2H); 5.07 (s, 2H).

Step-3: Synthesis of N-{3-[2,4-bis(benzyloxy)-6-fluorophenyl]oxetan-3-yl}-2-methylpropane-2-sulfinamide (A81): Compound A81 (1.5 g) was synthesized by following procedure as described for the synthesis of I-26 (step-3) using 1,5-bis(benzyloxy)-2-bromo-3-fluorobenzene (A80) (7.29 g, 18.8 mmol) as the starting material (toluene was used as the solvent). LCMS (ES) m/z: 484.1 [M+H]⁺.

Step-4: Synthesis of 3-[2,4-bis(benzyloxy)-6-fluorophenyl]oxetan-3-amine (I-29): Intermediate I-29 (1.8 g) was synthesized by following procedure as described for the synthesis of I-26 (step-4) using N-{3-[2,4-bis(benzyloxy)-6-fluorophenyl]oxetan-3-yl}-2-methylpropane-2-sulfinamide (A81) (3 g, 6.2 mmol) as the starting material. LCMS m/z: 363.0 [(M-NH₂)+H]⁺.

### Intermediate 30: Preparation of 1-(2,4-bis(methoxymethoxy)phenyl)cyclobutan-1-amine (I-30)

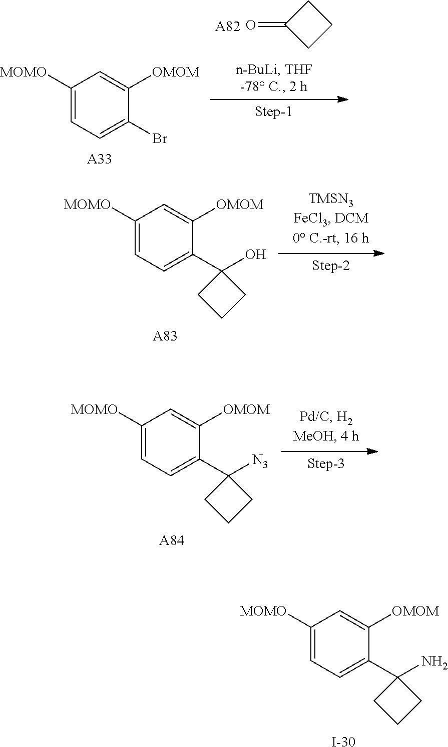

Step-1: Synthesis of 1-(2,4-bis(methoxymethoxy)phenyl)cyclobutan-1-ol (A83): A 2.5 M solution of n-BuLi (12.1 mL, 30.3 mmol) in heptane was added to a stirred solution of 1-bromo-2,4-bis(methoxymethoxy)benzene (A33) (7 g, 25.3 mmol) in anhydrous THE (70 mL) at −78° C. It was stirred for 5 min before addition of a solution of cyclobutanone (A82) (1.77 g, 25.3 mmol) in THE (10 mL). The reaction mixture was then allowed to warm to room temperature, while monitoring reaction progress by TLC. After completion of the reaction (2 h), the reaction mixture was quenched by addition of saturated NH₄Cl solution (50 mL) and extraction was carried out using EtOAc (3×70 mL). The combined organic extracts were washed with brine solution (3×30 mL), dried over anhydrous Na₂SO₄, filtered and concentrated under reduced pressure. The residue was purified by Combi-Flash (using gradient elution of 0-50% of EtOAc in heptane) to afford the desired compound A83 (4 g) as a pale yellow thick oil. ¹H NMR, (400 MHz, DMSO-d₆): δ 7.18 (d, J=8.4 Hz, 1H); 6.69 (d, J=2.4 Hz, 1H); 6.61-6.58 (m, 1H); 5.20 (s, 2H); 5.14 (s, 2H); 4.87 (s, 1H); 3.42 (s, 3H); 3.40 (s, 3H); 2.22-2.14 (m, 2H); 2.01-1.90 (m, 2H); 1.63-1.55 (m, 2H).

Step-2: Synthesis of 1-(1-azidocyclobutyl)-2,4-bis(methoxymethoxy)benzene (A84): To a stirred solution of 1-[2,4-bis(methoxymethoxy)phenyl]cyclobutan-1-ol (A83) (4 g, 14.9 mmol) in DCM (80 mL) was added azidotrimethylsilane (2.37 mL, 17.9 mmol) and FeCl₃ (242 mg, 1.49 mmol) at 0° C. and the reaction was allowed to warm to room temperature. After complete consumption of starting material (16 h), the reaction mixture was quenched with addition of saturated NH₄Cl solution (50 mL) and extraction was carried out using EtOAc (3×50 mL). The combined organic extracts were washed with brine solution (3×30 mL), dried over anhydrous Na₂SO₄, filtered and concentrated under reduced pressure. The residue was purified by Combi-Flash (using gradient elution of 0-100% of EtOAc in heptane) to afford desired compound A84 (3 g) as a pale yellow liquid. ¹H NMR, (400 MHz, CDCl₃): δ 7.07 (d, J=8.4 Hz 1H); 6.83 (d, J=2 Hz, 1H); 6.68-6.66 (m, 1H); 5.23-5.16 (m, 4H); 3.65-3.44 (m, 6H); 2.64-2.57 (m, 2H); 2.51-2.44 (m, 2H); 2.26-2.15 (m, 1H); 1.85-1.79 (m, 1H).

Step-3: Synthesis of 1-(2,4-bis(methoxymethoxy)phenyl)cyclobutan-1-amine (I-30): To a stirred solution of 1-(1-azidocyclobutyl)-2,4-bis(methoxymethoxy)benzene (A84) (3 g, 10.2 mmol) in MeOH (30 mL) was added 10% Pd/C (1.5 g) and the reaction mixture was stirred under H₂ atmosphere, while monitoring reaction progress by TLC. After completion of the reaction (4 h), the catalyst was filtered off through a celite pad and washed with MeOH (3×15 mL). The filtrate was concentrated under reduced pressure and the residue was triturated with diethyl ether (3×10 mL) to afford desired intermediate 1-30 (2 g).

¹H NMR, (400 MHz, DMSO-d₆): δ 7.02 (d, J=8.4 Hz, 1H); 6.66 (s, 1H); 6.58-6.56 (m, 1H); 5.17 (s, 2H); 5.11 (s, 2H); 3.37 (s, 3H); 3.34 (s, 3H); 2.34-2.32 (m, 2H); 2.07-2.00 (m, 4H).

### Intermediate 31: Preparation of tert-butyl (3-(aminomethyl)-2-fluorophenyl)carbamate (I-31)

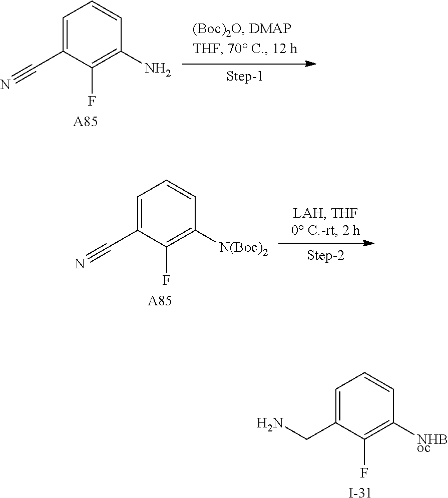

Step-1: Synthesis of tert-butyl N-[(tert-butoxy) carbonyl]-N-(3-cyano-2-fluorophenyl) carbamate (A86): To a stirred solution of 3-amino-2-fluorobenzonitrile (A85) (10 g, 73.5 mmol) in THE (150 mL, 1.84 mol) was added (Boc)₂O (42.2 mL, 184 mmol) and DMAP (2.26 g, 18.4 mmol) at room temperature. The reaction mixture was stirred at 70° C. for 16 h. The reaction mixture was then cooled to room temperature and water (50 mL) was added to it. Extraction was carried out using EtOAc (3×70 mL); the combined organic extracts were washed with brine solution (3×50 mL), dried over anhydrous Na₂SO₄, filtered and concentrated under reduced pressure. The residue was purified by Combi-Flash (using gradient elution of 0-16% of EtOAc in n-heptane) to afford desired compound A86 (20 g) as an off white solid. LCMS (ES) m/z: 237.0 [(M-Boc)+H]⁺.

Step-2: Synthesis of tert-butyl N-[3-(amino methyl)-2-fluorophenyl]carbamate (I-31): To a stirred solution of tert-butyl N-[(tert-butoxy)carbonyl]-N-(3-cyano-2-fluorophenyl)carbamate (A86) (10 g, 29.7 mmol) in anhydrous THE (0.2 L) was added a 2.0 M LAH solution in THE (2.26 g, 59.5 mmol) at 0° C. The reaction mixture was then allowed to warm to room temperature while monitoring reaction progress by TLC. After complete consumption of starting material (2 h), the reaction mixture was quenched with addition of hydrated Na₂SO₄. The reaction mixture was then filtered through a pad of celite and washed with EtOAc (3×100 mL). The filtrate was concentrated under reduced pressure and the residue was purified by Combi-flash (using gradient elution of 0-80% of EtOAc in n-heptane) to afford desired compound I-31 (4.7 g) as a white solid. LCMS (ES) m/z: 241.0 [M+H]⁺.

### Intermediate 32: Preparation of tert-butyl (2-fluoro-3-formylphenyl)carbamate (I-32)

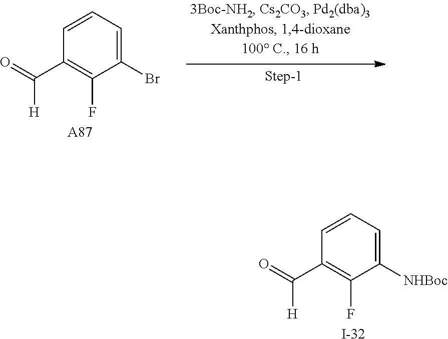

Step-1: Synthesis of tert-butyl N-(2-fluoro-3-formylphenyl) carbamate (I-32): Argon gas was purged through a stirred suspension of 3-bromo-2-fluorobenzaldehyde (A87) (5 g, 24.6 mmol), tert-butyl carbamate (3.46 g, 29.6 mmol) and Cs₂CO₃ (9.63 g, 29.6 mmol) in 1,4-dioxane (25 mL) for 10 min, before addition of Pd₂(dba)₃ (564 mg, 616 μmol) and Xantphos (855 mg, 1.48 mmol). The reaction mixture was then stirred at 100° C. for 16 h. After complete consumption of starting material (16 h), the reaction mixture was cooled to room temperature and water (50 mL) was added to it. Extraction was carried out using EtOAc (2×40 mL); the combined organic extracts were washed with brine solution (40 mL), dried over anhydrous Na₂SO₄, filtered and concentrated under reduced pressure. The residue was purified by Combi-Flash (using gradient elution of 0-40% of EtOAc in heptane) to afford desired compound I-32 (1.75 g) as a white solid. LCMS (ES) m/z: 140.0 [(M-Boc)+H]⁺.

### Intermediate 33: Preparation of 1-(2-fluoro-3-formylphenyl)-N-methylmethanesulfonamide (I-33)

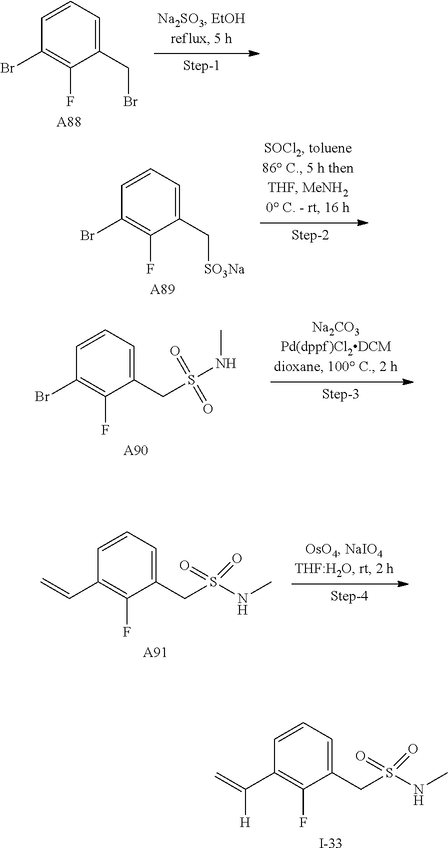

Step-1: Synthesis of sodium (3-bromo-2-fluorophenyl)methanesulfonate (A89): To a stirred solution of 1-bromo-3-(bromomethyl)-2-fluorobenzene (A88) (6 g, 22.4 mmol) in EtOH (80 mL) and water (80 mL) was added sodium sulfite (2.77 g, 26.9 mmol); and the reaction mixture was allowed to stir at 90° C., while monitoring reaction progress by TLC. After completion of reaction (5 h), it was concentrated under reduced pressure and dried to afford the desired compound A89 (6.7 g) as a colorless solid, which was carried forward for the next step. LCMS (ES) m/z: 268.7 [M+H]⁺ (the corresponding acid mass).

Step-2: Synthesis of 1-(3-bromo-2-fluorophenyl)-N-methylmethanesulfonamide (A90): To a stirred suspension of sodium (3-bromo-2-fluorophenyl)methanesulfonate (A89) (7.5 g, 25.8 mmol) in toluene (50 mL) was added SOCl₂ (8 mL) and the reaction mixture was allowed to stir at 85° C. for 5 h. It was then cooled to room temperature and concentrated under reduced pressure. The residue was dissolved in THE (15 mL) and added to a solution of methylamine (8.6 mL, 17.2 mmol) in THE (10 mL) at 0° C. The reaction mixture was allowed to stir at room temperature for 16 h. Water (50 mL) was added to it and extraction was carried out using EtOAc (2×40 mL). The combined organic extracts were washed with brine solution (2×20 mL), dried over anhydrous Na₂SO₄, filtered and concentrated under reduced pressure. The residue was purified by Combi-Flash (using gradient elution of 0-35% of EtOAc in heptane) to afford the desired compound A90 (4.5 g) as a white solid. ¹H NMR (400 MHz, DMSO-d₆): δ 7.72-7.68 (m, 1H); 7.46-7.42 (m, 1H); 7.21-7.13 (m, 2H); 4.42 (s, 2H); 2.61 (d, J=4.8 Hz, 3H).

Step-3: Synthesis of 1-(3-ethenyl-2-fluorophenyl)-N-methylmethanesulfonamide (A91): Argon gas was purged through a stirred suspension of 1-(3-bromo-2-fluorophenyl)-N-methylmethanesulfonamide (A90) (3.5 g, 12.4 mmol), aqueous solution of Na₂CO₃ (3.94 g, 37.2 mmol, in 7 mL water) and potassium ethenyltrifluoroboranuide (2.16 g, 16.1 mmol) in 1,4-dioxane (28 mL) for 10 min before addition of Pd(dppf)Cl₂— CH₂Cl₂ (1.01 g, 1.24 mmol). The reaction mixture was then stirred at 100° C., while monitoring reaction progress by TLC. After complete consumption of starting material (2 h), the reaction mixture was cooled to room temperature, filtered through celite pad and washed with EtOAc (3×20 mL). The filtrate was diluted with water (30 mL) and extraction was carried out using EtOAc (2×30 mL). The combined organic extracts were washed with brine solution (2×20 mL), dried over anhydrous Na₂SO₄, filtered and concentrated under reduced pressure. The residue was purified by Combi-Flash (using gradient elution of 0-40% of EtOAc in heptane) to afford desired compound A91 (2.8 g) as an off-white solid. LCMS (ES) m/z: 228.0 [M−H]⁺.

Step-4: Synthesis of 1-(3-ethenyl-2-fluorophenyl)-N-methylmethanesulfonamide (I-33): To a stirred solution of 1-(3-ethenyl-2-fluorophenyl)-N-methylmethanesulfonamide (A91) (3.7 g, 16.1 mmol) in THE (37 mL) and water (18 mL) mixture was added OsO₄ (4% in water) (5 mL, 0.80 mmol) and the reaction mixture was allowed to stir at room temperature for 30 min. To this was then added NaIO₄ (6.9 g, 32.3 mmol) and the reaction mixture was allowed to stir at room temperature, while monitoring reaction progress by TLC/LCMS. After complete consumption of starting material (2 h), water (50 mL) was added to it and extraction was carried out using EtOAc (2×30 mL). The combined organic extracts were washed with brine solution (30 mL), dried over anhydrous Na₂SO₄, filtered and concentrated under reduced pressure. The crude was purified by Combi-flash (using gradient elution of 0-40% of EtOAc in n-heptane) to afford desired compound I-33 (1.7 g) as an off white solid. LCMS (ES) m/z: 230.1 [M−H]⁺.

### Intermediate 34: Preparation of benzyl (2-fluoro-3-formylphenyl)carbamate (I-34)

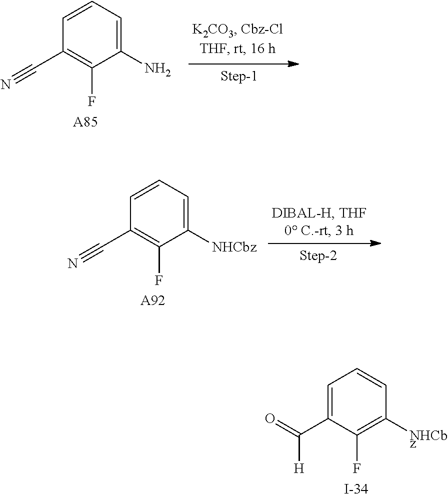

Step-1: Synthesis of benzyl (3-cyano-2-fluorophenyl)carbamate (A92): To a stirred solution of 3-amino-2-fluorobenzonitrile (A85) (5 g, 36.7 mmol) in THF (50 mL) under nitrogen atmosphere was added K₂CO₃ (15.2 g, 110 mmol) and benzyl chloroformate (15.8 mL, 110 mmol) at 0° C. The reaction mixture was stirred at room temperature, while monitoring reaction progress by TLC/LCMS. After completion of the reaction (16 h), water (20 mL) was added to it and extraction was carried out using EtOAc (3×50 mL). The combined organic extracts were washed with saturated sodium bicarbonate solution (2×30 mL), brine solution (2×30 mL), dried over anhydrous Na₂SO₄, filtered and concentrated under reduced pressure. The residue was purified by Combi-Flash column (using gradient elution of 0-20% of EtOAc in heptane) to afford desired compound A92 (9.3 g) as an off-white solid. LCMS (ES) m/z: 271.1 [M+H]⁺.

Step-2: Synthesis of benzyl (2-fluoro-3-formylphenyl)carbamate (I-34): To a stirred solution of benzyl N-(3-cyano-2-fluorophenyl)carbamate (A92) (1 g, 3.7 mmol) in anhydrous THF (20 mL) under nitrogen atmosphere was added 1 M DIBAL-H in THF (18.5 mL, 18.5 mmol) at 0° C. and the reaction mixture was allowed to warm to room temperature. The reaction was monitored by TLC/LCMS. After complete consumption of starting material (3 h), reaction mixture was quenched with addition of saturated NH₄Cl solution (10 mL) and extraction was carried out using EtOAc (3×30 mL). the combined organic extracts were washed with water (2×30 mL), saturated NaHCO₃ solution (2×20 mL), brine solution (2×30 mL), dried over anhydrous Na₂SO₄, filtered and concentrated under reduced pressure. The residue was triturated using DCM-n-heptane (1:4) followed by diethyl ether (2×10 mL) to afford desired compound I-34 (230 mg) as a white solid. LCMS (ES) m/z: 274.1 |M+H|⁺.

### Intermediate 35: Preparation of benzyl (2-fluoro-3-formylphenyl)carbamate (I-35)

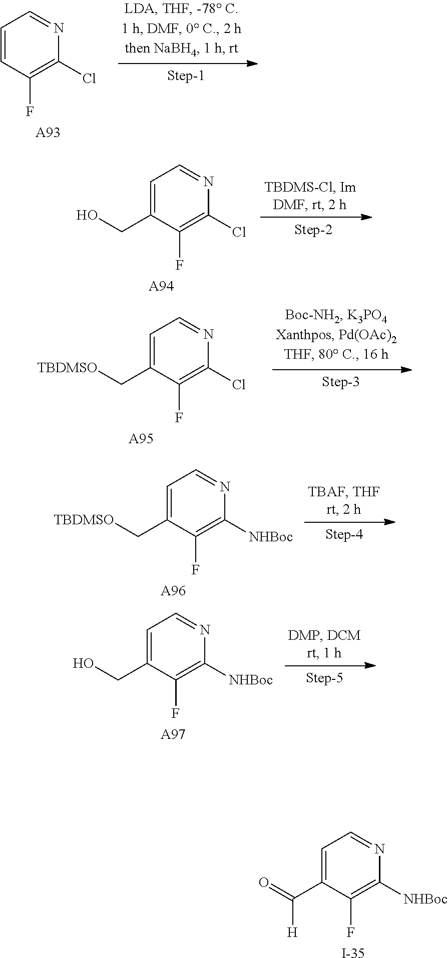

Step-1: Synthesis of (2-chloro-3-fluoropyridin-4-yl)methanol (A94): To a stirred solution of 2-chloro-3-fluoropyridine (A93) (8 g, 60.8 mmol) in THF (20 mL) was added LDA (2.0 M in THF) (6.52 g, 30.4 mL, 60.8 mmol) at −78° C. and stirred for 2 h. To this was then DMF (44.5 g, 608 mmol) at −78° C. and the reaction mixture was allowed to warm to room temperature over 2 h. To this was then added NaBH₄ (2.99 g, 79.1 mmol) at 0° C. and stirred for 1 h. The reaction mixture was then quenched with water (50 mL) and extraction was carried out using EtOAc (3×70 mL). The combined organic extracts were washed with aqueous 1 M HCl solution (2×20 mL), saturated NaHCO₃ solution (3×30 mL), brine solution (2×25 mL), dried over anhydrous Na₂SO₄, filtered and concentrated under reduced pressure. The residue was purified by Combi-Flash (using gradient elution of 0-6% of MeOH in DCM) to afford the desired compound A94 (7.8 g) as a pale yellow solid. LCMS (ES) m/z: 162.1 [M+H]⁺.

Step-2: Synthesis of 4-{[(tert-butyldimethylsilyl)oxy]methyl}-2-chloro-3-fluoropyridine (A95): To a stirred solution of (2-chloro-3-fluoropyridin-4-yl)methanol (A94) (8 g, 49.5 mmol) in DMF (80 mL) was added imidazole (10.1 g, 149 mmol) and TBDMS-CI (14.9 g, 99 mmol) at 0° C. The reaction mixture was stirred at room temperature, while monitoring reaction progress by TLC/LCMS analysis. After complete consumption of starting material (2 h), the reaction mixture was quenched with addition of water (50 mL) and extraction was carried out using DCM (3×50 mL). The combined organic extracts were washed with cold water (3×50 mL), brine solution (2×50 mL), dried over anhydrous Na₂SO₄, filtered and concentrated under reduced pressure. The residue was purified by Combi-Flash (using gradient elution of 0-2% of EtOAc in DCM) to afford the desired compound A95 (10 g) as an off-white solid. LCMS (ES) m/z: 276.2 [M+H]⁺.

Step-3: Synthesis of tert-butyl-4-{[(tert-butyl)bis(methyl)siloxy]methyl}-3-fluoro-2-pyridylaminoformylate (A96): Argon gas was purged through a stirred suspension of 4-{[(tert-butyldimethylsilyl)oxy]methyl}-2-chloro-3-fluoropyridine (A95) (5 g, 18.1 mmol), Boc-NH₂ (3.4 g, 29 mmol) and K₃PO₄ (12.3 g, 58 mmol) in THF (50 mL) for 10 min before addition of Xantphos (1.57 g, 2.72 mmol) and Pd(OAc)₂ (610 mg, 2.72 mmol). The reaction mixture was then stirred at 80° C. for 16 h. It was then cooled to room temperature and water (25 mL) was added to it. Extraction was carried out using EtOAc (3×50 mL); the combined organic extracts were washed with brine solution (2×30 mL), dried over anhydrous Na₂SO₄, filtered and concentrated under reduced pressure. The residue was purified by Combi-Flash (using gradient elution of 0-70% of EtOAc in hexane) to afford the desired compound A96 (2.2 g, 6.16 mmol) as a pale yellow solid. LCMS (ES) m/z: 357.2 [M+H]⁺.

Step-4: Synthesis of tert-butyl N-[3-fluoro-4-(hydroxymethyl)pyridin-2-yl]carbamate (A97): To a stirred solution of tert-butyl N-(4-{[(tert-butyldimethylsilyl)oxy]methyl}-3-fluoropyridin-2-yl)carbamate (A96) (8.5 g, 23.8 mmol) in THF (85 ml) was added 1.0 M TBAF in THE (9.35 g, 35.8 mmol) at 0° C.; and the reaction mixture was stirred at room temperature, while monitoring reaction progress by TLC/LCMS. After complete consumption of starting material (2 h), the reaction mixture was quenched with ice water (50 mL) and extraction was carried out using EtOAc (3×50 mL). The combined organic extracts were washed with brine solution (2×30 mL), dried over anhydrous Na₂SO₄, filtered and concentrated under reduced pressure. The residue was purified by Combi-Flash (using gradient elution of 0-80% of EtOAc in heptane) to afford desired compound A97 (4.5 g, 18.6 mmol) as a pale yellow viscous oil. LCMS (ES) m/z: 243.1 [M+H]⁺.

Step-5: Synthesis of tert-butyl N-(3-fluoro-4-formylpyridin-2-yl)carbamate (I-35): To a stirred solution of tert-butyl N-[3-fluoro-4-(hydroxymethyl)pyridin-2-yl]carbamate (A97) (4.5 g, 18.6 mmol) in DCM (150 mL) was added DMP (11.8 g, 27.9 mmol) at 0° C. and the reaction mixture was stirred at room temperature. The reaction progress was monitored by TLC/LCMS analysis. After complete consumption of starting material (2 h), cold water (50 mL) was added to it and extraction was carried out using DCM (3×70 mL). The combined organic extracts were washed with brine solution (2×50 mL), dried over anhydrous Na₂SO₄, filtered and concentrated under reduced pressure. The residue was purified by Combi-Flash (using gradient elution of 0-55% of EtOAc in hexane) to afford the desired compound I-35 (3.3 g) as an off-white solid. LCMS (ES) m/z: 185.1 |(M-^{t}Bu)+H|⁺.

### Intermediate 36: Preparation of 2-(cyclopropylsulfonylamino)-3-fluoroisonicotinaldehyde (I-36)

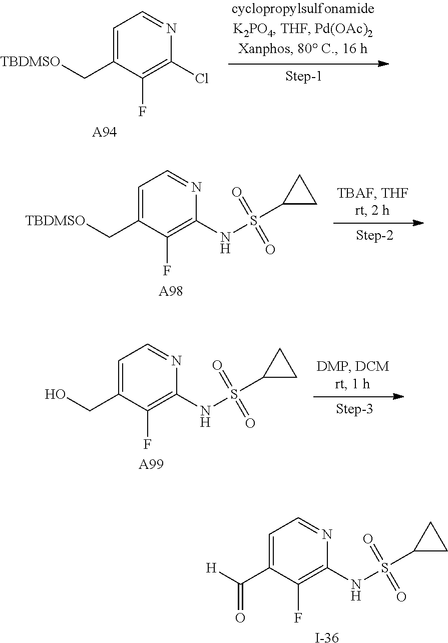

Step-1: Synthesis of (cyclopropylsulfonyl)(4-{[(tert-butyl)bis(methyl)siloxy]methylI-3-fluoro-2-pyridyl)amine (A98): Compound A98 (2.1 g) was synthesized by following procedure as described for the synthesis of I-35 (step-3) using (tert-butyl)[(2-chloro-3-fluoro-4-pyridyl)methoxy]bis(methyl)silane (A94) (10 g, 36.3 mmol) and cyclopropanesulfonamide (6.6 g, 54.4 mmol) as the starting materials. LCMS (ES) m/z: 361.1 [M+H]⁺.

Step-2: Synthesis of [2-(cyclopropylsulfonylamino)-3-fluoro-4-pyridyl]methanol (A99): Compound A99 (2.0 g) was synthesized by following procedure as described for the synthesis of I-35 (step-4) using (cyclopropylsulfonyl)(4-{[(tert-butyl)bis(methyl)siloxy]methyl}-3-fluoro-2-pyridyl)amine (A98) (4.1 g, 11.4 mmol) as the starting material. LCMS (ES) m/z: 247.1 [M+H]⁺.

Step-3: Synthesis of 2-(cyclopropylsulfonylamino)-3-fluoroisonicotinaldehyde (I-36): Intermediate I-36 (1.4 g) was synthesized by following procedure as described for the synthesis of I-35 (step-5) using [2-(cyclopropylsulfonylamino)-3-fluoro-4-pyridyl]methanol (A99) as the starting material (2.0 g, 8.12 mmol). LCMS (ES) m/z: 245.1 [M+H]⁺.

### Intermediate 37: Preparation of 2-fluoro-3-[(2-oxo-1-imidazolidinyl)methyl]benzaldehyde (I-37)

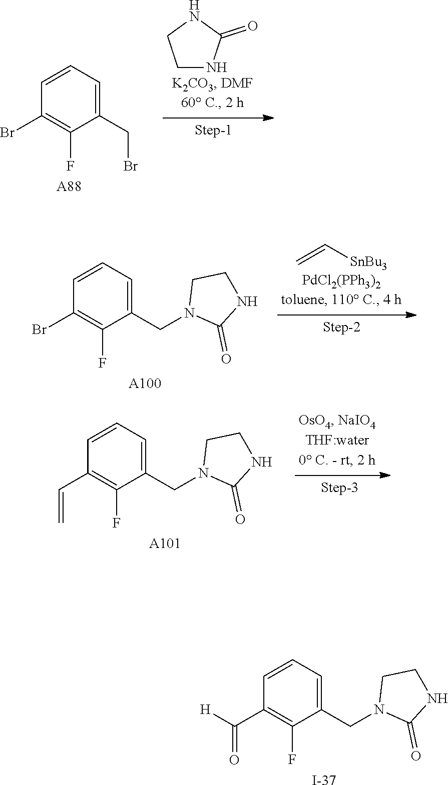

Step-1: Synthesis of 1-[(3-bromo-2-fluorophenyl)methyl]-2-imidazolidinone (A100): To a stirred solution of 2-imidazolidinone (5 g, 58.1 mmol) in DMF (125 mL) was added NaH (60% dispersion in mineral oil) (2.32 g, 58.1 mmol) at 0° C. and stirred for 10 min before addition of 1-bromo-3-(bromomethyl)-2-fluorobenzene (A88) (7.78 g, 29 mmol). The reaction mixture was allowed to warm to room temperature and the reaction progress was monitored by TLC/LCMS analysis. After completion of the reaction (3 h), it was quenched with water (20 mL) and extraction was carried out using DCM (3×50 mL). The combined organic extracts were washed with brine solution (2×25 mL), dried over anhydrous Na₂SO₄, filtered and concentrated under reduced pressure. The residue was purified by Combi-Flash (using gradient elution of 0-80% of EtOAc in heptane) to afford the desired compound A100 (4.5 g) as a colorless thick oil. LCMS (ES) m/z: 273.6 [M+H]⁺.

Step-2: Synthesis of 1-(2-fluoro-3-vinylbenzyl)imidazolidin-2-one (A101): Argon gas was purged through a stirred solution of 1-[(3-bromo-2-fluorophenyl)methyl]-2-imidazolidinone (A100) (2.7 g, 9.89 mmol) and tributyl(vinyl)stannane (3.13 g, 9.89 mmol) in toluene (50.5 mL) for 5 min before addition of PdCl₂(PPh₃)₂ (27.7 mg, 39.5 μmol). The reaction mixture was then stirred at 100° C., while monitoring reaction progress by TLC. After completion of the reaction (4 h), it was cooled to room temperature and volatiles were removed under reduced pressure. The residue was purified by Combi-Flash (using gradient elution of 0-50% of EtOAc in heptane) to afford desired compound A101 (2.4 g) as a pale yellow liquid. LCMS (ES) m/z: 221.6 [M+H]⁺.

Step-3: Synthesis of 2-fluoro-3-[(2-oxo-1-imidazolidinyl)methyl]benzaldehyde (I-37): To a stirred solution of 1-(2-fluoro-3-vinylbenzyl)imidazolidin-2-one (A101) (2.4 g, 10.9 mmol) in THE (30 mL) and water (10 mL) was added OsO₄ (4% in water) (3.5 mL, 545 μmol). To this was then added NaIO₄ (4.66 g, 21.8 mmol) and the reaction mixture was stirred at room temperature, while monitoring reaction progress by TLC. After completion of the reaction (2 h), water (30 mL) was added to it and extraction was carried out with EtOAc (2×30 mL). The combined organic extracts were washed with brine solution (2×30 mL), dried over anhydrous Na₂SO₄, filtered and concentrated under reduced pressure. The residue was purified by Combi-flash (using gradient elution of 0-40% of EtOAc in heptane) to afford the desired compound I-37 (2 g) as a pale yellow thick oil. LCMS (ES) m/z: 223.3 [M+H]⁺.

### Intermediate 38: Preparation of tert-butyl (4-(aminomethyl)-3-fluoropyridin-2-yl)carbamate (I-38)

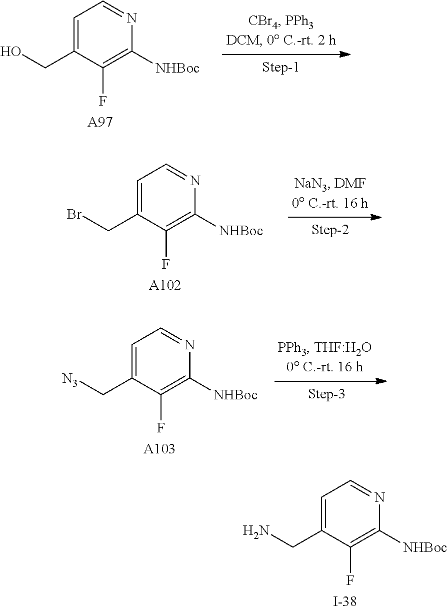

Step-1: Synthesis of 4-(bromomethyl)-3-fluoro-2-pyridylamino-tert-butylformylate (A102): To a stirred solution of tert-butyl-3-fluoro-4-(hydroxymethyl)-2-pyridylaminoformylate (A97) (2.4 g, 9.91 mmol) in DCM (100 mL) was added PPh₃ (3.9 g, 14.9 mmol) and CBr₄ (4.6 g, 13.9 mmol) at 0° C. The reaction mixture was stirred at room temperature and the reaction progress was monitored by TLC/LCMS analysis. After complete consumption of starting material (2 h), water (50 mL) was added to it and extraction was carried out by using DCM (2×50 mL). The combined organic extracts were washed with brine solution (2×50 mL), dried over anhydrous Na₂SO₄, filtered and concentrated under reduced pressure. The residue was purified by Combi-Flash (using gradient elution of 0-40% of EtOAc in hexane) to afford the desired compound A102 (2.6 g) as a pale yellow viscous liquid. LCMS (ES) m/z: 305.0 [M+H]⁺.

Step-2: Synthesis of 4-(azidomethyl)-3-fluoro-2-pyridylamino-tert-butylformylate (A103): To a stirred solution of 4-(bromomethyl)-3-fluoro-2-pyridylamino-tert-butylformylate (A102) (2.6 g, 8.52 mmol) in DMF (26 mL) was added sodium azide (665 mg, 10.2 mmol) at 0° C. and the reaction mixture was allowed to warm to room temperature over 16 h. After complete consumption of starting material (16 h), water (50 mL) was added to it and extraction was carried out with EtOAc (2×50 mL). The combined organic extracts were washed with brine solution (2×25 mL), dried over anhydrous Na₂SO₄, filtered and concentrated under reduced pressure to afford desired compound A103 (2.3 g) as a pale yellow viscous liquid. LCMS m/z: 268.0 [M+H]⁺.

Step-3: Synthesis of tert-butyl (4-(aminomethyl)-3-fluoropyridin-2-yl) carbamate (I-38): To a stirred solution of 4-(azidomethyl)-3-fluoro-2-pyridylamino-tert-butylformylate (A103) (2.3 g, 8.61 mmol) in THE (24 mL) and water (6.1 mL) was added PPh₃ (3.39 g, 12.9 mmol) at 0° C. The reaction mixture was stirred at room temperature for 16 h. After complete consumption of starting material, volatiles were removed under reduced pressure and the residue was purified by Combi-Flash (using gradient elution of 0-5% MeOH in DCM) to afford desired compound I-38 (1.5 g) as an off-white solid. LCMS m/z: 242.1 [M+H]⁺.

### Intermediate 39: Preparation of 4-(1-aminocyclobutyl)benzene-1,3-diol (I-39)

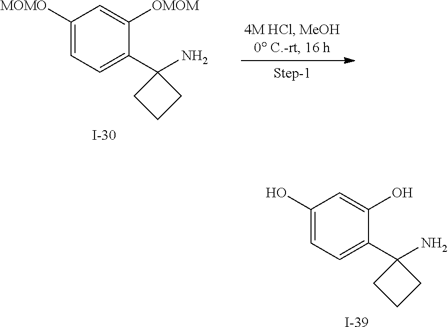

Step-1: Synthesis of 4-(1-aminocyclobutyl)benzene-1,3-diol (I-39): To a stirred solution of 1-[2,4-bis(methoxymethoxy)phenyl]cyclobutylamine (I-30) (2.8 g, 10.5 mmol) in MeOH (20 mL) was added a 4 M solution of HCl in 1,4-dioxane (20 mL) at 0° C. The reaction mixture was then stirred at room temperature for 16 h. After completion of the reaction, volatiles were removed under reduced pressure and the residue was washed with diethyl ether (3×10 mL) to afford desired intermediate I-39 (1.8 g) as an off-white solid. ¹H NMR (400 MHz, DMSO-d₆) δ 10.12 (s, 1H); 9.49 (s, 1H); 8.08 (br s, 3H); 7.06 (d, J=8.4 Hz, 1H); 6.45 (s, 1H); 6.27-6.25 (m, 1H); 3.63-3.56 (m, 2H); 2.09-1.98 (m, 2H); 1.70-1.68 (m, 2H).

### Intermediates 40 and 41: Preparations of 1-(5-fluoro-2,4-bis(methoxymethoxy)phenyl)cyclobutan-1-amine (I-40) and 4-(1-aminocyclobutyl)-6-fluorobenzene-1,3-diol (I-41)

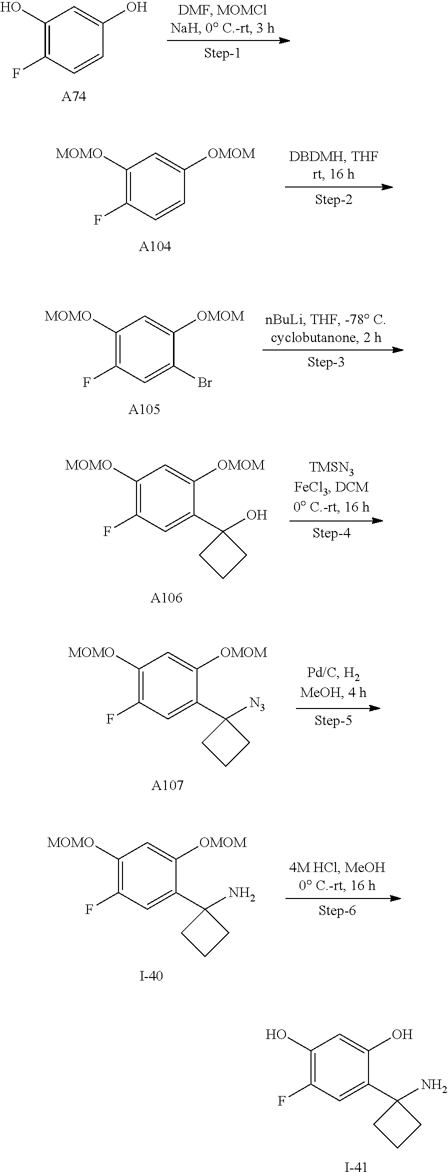

Step-1: Synthesis of 1-fluoro-2,4-bis(methoxymethoxy)benzene (A104): Compound A104 (10 g) was synthesized by following procedure as described for the synthesis of I-16 (step-1) using 4-fluorobenzene-1,3-diol (A74) (10 g, 78.1 mmol) as the starting material. ¹H NMR (400 MHz, CDCl₃): δ 7.03-6.98 (m, 1H); 6.93-6.90 (m, 1H); 6.68-6.64 (m, 1H); 5.21 (s, 2H); 5.13 (s, 2H); 3.54 (s, 3H); 3.49 (s, 3H).

Step-2: Synthesis of 1-bromo-5-fluoro-2,4-bis(methoxymethoxy)benzene (A105): Compound A105 (10 g) was synthesized by following procedure as described for the synthesis of I-26 (step-2) using 1-fluoro-2,4-bis(methoxymethoxy)benzene (A104) (10 g, 46.3 mmol) as the starting material.

Step-3: Synthesis of 1-(5-fluoro-2,4-bis(methoxymethoxy)phenyl)cyclobutan-1-ol (A106): Compound A106 (1.75 g) was synthesized by following procedure as described for the synthesis of I-30 (step-1) using 1-bromo-5-fluoro-2,4-bis(methoxymethoxy)benzene (A105) (5 g, 16.9 mmol) as the starting material. ¹H NMR (400 MHz, CDCl₃) δ 7.11 (d, J=12.4 Hz, 1H); 6.94 (d, J=7.6 Hz, 1H); 5.22-5.16 (m, 4H); 5.09 (s, 1H); 3.42-3.41 (m, 6H); 2.55-2.51 (m, 2H); 2.20-2.14 (m, 2H); 1.97-1.91 (m, 2H).

Step-4: Synthesis of 1-(1-azidocyclobutyl)-5-fluoro-2,4-bis(methoxymethoxy)benzene (A107): Compound A107 (2.75 g) was synthesized by following procedure as described for the synthesis of 1-30 (step-2) using 1-[5-fluoro-2,4-bis(methoxymethoxy)phenyl]cyclobutanol (A106) (3.5 g, 12.2 mmol) as the starting material.

¹H NMR (400 MHz, CDCl₃) δ 7.11 (d, J=8.8 Hz, 1H); 6.73 (d, J=2.8 Hz, 1H); 6.65-6.62 (m, 1H); 5.17-5.15 (m, 4H); 3.37-3.35 (m, 6H); 2.64-2.54 (m, 2H); 2.40-2.34 (m, 2H); 2.11-2.04 (m, 1H); 1.76-1.69 (m, 1H).

Step-5: Synthesis of 1-(5-fluoro-2,4-bis(methoxymethoxy)phenyl)cyclobutan-1-amine (I-40): Intermediate I-40 (4.5 g) was synthesized by following procedure as described for the synthesis of 1-30 (step-3) using 1-(1-azidocyclobutyl)-5-fluoro-2,4-bis(methoxymethoxy)benzene (A107) (5.5 g, 17.7 mmol) as the starting material. ¹H NMR (400 MHz, DMSO-d₆) δ 6.98-6.91 (m, 2H); 5.22-5.17 (m, 4H); 3.48-3.35 (m, 6H); 2.50-2.40 (m, 3H); 2.22-2.01 (m, 3H).

Step-6: Synthesis of 4-(1-aminocyclobutyl)-6-fluorobenzene-1,3-diol (1-41): Intermediate I-41 (2.8 g) was synthesized by following procedure as described for the synthesis of 1-39 (step-1) using 1-[5-fluoro-2,4-bis(methoxymethoxy)phenyl]cyclobutylamine (I-40) (4 g, 14 mmol) as the starting material.

¹H NMR (400 MHz, DMSO-d₆) δ 10.07 (s, 1H); 9.96 (s, 1H); 8.12 (br s, 2H); 7.04 (d, J=12.4 Hz, 1H); 6.65 (d, J=8.0 Hz, 1H); 3.56 (s, 2H); 2.46-2.40 (m, 2H); 2.10-2.06 (m, 1H); 1.74-1.68 (m, 1H).

### Intermediate 42: Preparation of 4-(2-aminopropan-2-yl)benzene-1,3-diol (I-42)

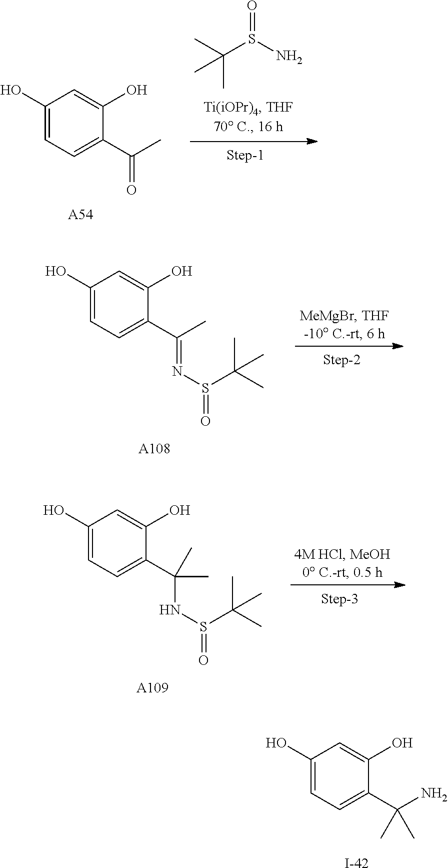

Step-1: Synthesis of N-[(1Z)-1-(2,4-dihydroxyphenyl)ethylidene]-2-methylpropane-2-sulfinamide (A108): To a stirred solution of 1-(2,4-dihydroxyphenyl)ethan-1-one (A54) (10 g, 65.7 mmol) and 2-methylpropane-2-sulfinamide (23.9 g, 197 mmol) in THF (120 mL) was added titanium (IV) isopropoxide (117 mL, 394 mmol) at room temperature and the reaction mixture was stirred at 70° C. for 16 h. After completion of the reaction, it was cooled to room temperature and quenched with addition of ice-cold water (50 mL). It was then filtered through celite and washed with EtOAc (50 mL). The filtrate was extracted using EtOAc (3×50 mL); the combined organic extracts were washed with brine solution (2×50 mL), dried over anhydrous Na₂SO₄, filtered and concentrated under reduced pressure. The residue was purified by Combi-Flash (using gradient elution of 0-40% of EtOAc in heptane) to afford the desired compound A108 (4.51 g) as a grey solid. LCMS (ES) m/z: 254.15 [M−H]⁺.

Step-2: Synthesis of N-[2-(2,4-dihydroxyphenyl)propan-2-yl]-2-methylpropane-2-sulfinamide (A109): To a stirred solution of N-[(1Z)-1-(2,4-dihydroxyphenyl)ethylidene]-2-methylpropane-2-sulfinamide (A108) (4.5 g, 17.6 mmol) in THF (110 mL) was added MeMgBr (16.8 g, 141 mmol) at −10° C. and the reaction mixture was allowed to warm to room temperature. After completion of the reaction (6 h), the reaction mixture was quenched with addition of saturated ammonium chloride solution (30 mL) and extraction was carried out using EtOAc (3×40 mL). The combined organic extracts were washed with brine solution (2×30 mL), dried over anhydrous Na₂SO₄, filtered and concentrated under reduced pressure. The residue was purified by Combi-Flash (using gradient elution of 0-30% of EtOAc in heptane) to afford the desired compound A109 (1.2 g) as a brown solid. LCMS (ES) m/z: 270.16 [M−H]⁺.

Step-3: Synthesis of 4-(2-aminopropan-2-yl)benzene-1,3-diol (I-42): To a stirred solution of N-[2-(2,4-dihydroxyphenyl)propan-2-yl]-2-methylpropane-2-sulfinamide (A109) (1.2 g, 4.42 mmol) in MeOH (15 mL) was added a 4 M solution of HCl in 1,4-dioxane (5.2 mL, 22.1 mmol) at 0° C. The reaction mixture was stirred at room temperature and the reaction progress was monitored by TLC/LCMS analysis. After completion of the reaction (0.5 h), the reaction mixture was concentrated under reduced pressure and the residue was washed with diethyl ether (3×10 mL) to afford desired compound I-42 (0.37 g) as a yellow solid. LCMS (ES) m/z: 166.15 [M−H]⁺.

### Intermediate 43: Preparation of 4-(2-aminopropan-2-yl)benzene-1,3-diol (I-43)

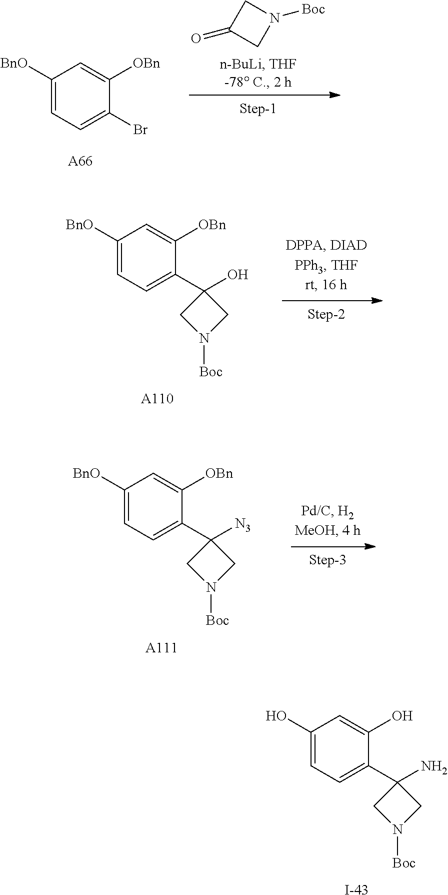

Step-1: Synthesis of tert-butyl 3-[2,4-bis(benzyloxy)phenyl]-3-hydroxyazetidine-1-carboxylate (A110): To a stirred solution of 2,4-bis(benzyloxy)-1-bromobenzene (A66) (10 g, 27.1 mmol) in anhydrous THE (150 mL) was added a solution of n-BuLi (2.2 M in hexane, 14.8 mL, 32.5 mmol) at −78° C. under nitrogen atmosphere. The reaction was stirred for 60 min before addition of a solution of tert-butyl 3-oxoazetidine-1-carboxylate (6.95 g, 40.6 mmol) in THE (21 mL). The reaction mixture was then allowed to warm to room temperature, while monitoring reaction progress by TLC. After completion of the reaction (2 h), it was quenched by addition of saturated ammonium chloride (50 mL) solution. Extraction was carried out using EtOAc (3×70 mL); the combined organic extracts were washed with brine solution (2×50 mL), dried over anhydrous Na₂SO₄, filtered and concentrated under reduced pressure. The residue was purified by Combi-Flash (using gradient elution of 0-30% of EtOAc in heptane) to afford desired compound A110 (3.2 g) as pale yellow solid. LCMS (ES) m/z: 462.20 [M+H]⁺.

Step-2: Synthesis of tert-butyl 3-azido-3-[2,4-bis(benzyloxy)phenyl]azetidine-1-carboxylate (A111): The solution of tert-butyl 3-[2,4-bis(benzyloxy)phenyl]-3-hydroxyazetidine-1-carboxylate (A110) (7 g, 15.2 mmol) in THE (140 mL) was added PPh₃ (7.16 g, 27.3 mmol), DIAD (4.47 mL, 22.7 mmol) and diphenylphosphinoyl azide (5.19 mL, 27.3 mmol). The reaction mixture was stirred at room temperature for 16 h. After completion of the reaction, ice water (50 mL) was added to it and extraction was carried out with EtOAc (3×50 mL). The combined organic extracts were washed with brine solution (2×30 mL), dried over anhydrous Na₂SO₄, filtered and concentrated under reduced pressure. The residue was purified by Combi-Flash (using gradient elution of 0-20% of EtOAc in heptane) to afford the desired compound A111 (4 g) as colorless sticky oil. LCMS (ES) m/z: 487.12 [M+H]⁺.

Step-3: Synthesis of tert-butyl 3-amino-3-(2,4-dihydroxyphenyl)azetidine-1-carboxylate (I-43): The a solution of tert-butyl 3-azido-3-[2,4-bis(benzyloxy)phenyl]azetidine-1-carboxylate (A111) (4.1 g, 8.43 mmol) in MeOH (50 mL) was added 5% Pd/C (1.79 g) and the reaction mixture was allowed to stir at room temperature under hydrogen atmosphere. The reaction progress was monitored by TLC/LCMS. After complete consumption of starting material (16 h), the catalyst was filtered off through a celite pad and washed with MeOH (2×5 mL). MeOH was then evaporated under reduced pressure and the residue was purified by Combi-Flash (using gradient elution of 0-80% of EtOAc in heptane) to afford the desired 1-43 (1.8 g) as an off-white solid. LCMS (ES) m/z: 281.13 |M+H|⁺.

### Intermediate 44: Preparation of 2-(benzyloxy)-4-((1-methyl-1H-pyrazol-3-yl)oxy)benzaldehyde (I-44)

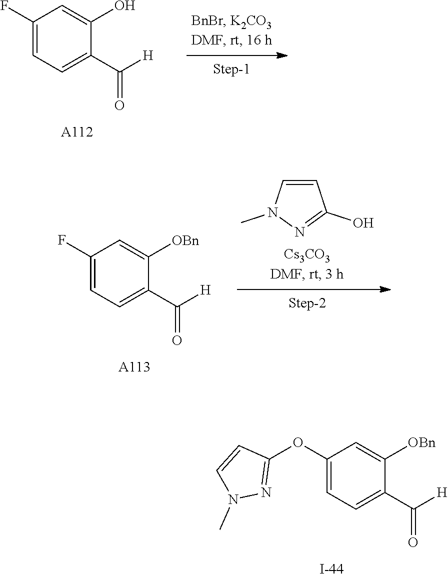

Step-1: Synthesis of 2-(benzyloxy)-4-fluorobenzaldehyde (A113): To a stirred solution of 4-fluoro-2-hydroxybenzaldehyde (A112) (10.0 g, 71.4 mmol) in DMF (40 mL) was added K₂CO₃ (29.6 g, 214 mmol) and (bromomethyl)benzene (12.7 mL, 107 mmol) at room temperature. The reaction progress was monitored by TLC/LCMS analysis. After complete consumption of starting material (16 h), cold water (50 mL) was added to it and extraction was carried out using EtOAc (3×70 mL). The combined organic extracts were washed with brine solution (2×50 mL), dried over anhydrous Na₂SO₄, filtered and concentrated under reduced pressure. The residue was purified by Combi-Flash (using gradient elution of 0-50% of EtOAc in heptane) to afford desired compound A113 (13.0 g) as a colorless liquid. ¹H NMR (400 MHz, DMSO-d₆): δ 10.32 (s, 1H); 7.79 (t, J=7.6 Hz, 1H); 7.53-7.52 (m, 2H); 7.43 (t, J=7.6 Hz, 1H); 7.38-7.35 (m, 2H); 7.26 (d, J=10.0 Hz, 1H); 6.94 (t, J=8.4 Hz, 1H); 5.31 (s, 2H).

Step-2: Synthesis of 2-(benzyloxy)-4-(1-methyl-3-pyrazolyloxy)benzaldehyde (I-44): To a stirred solution of 1-methyl-3-pyrazolol (1.94 g, 19.8 mmol) and 2-(benzyloxy)-4-fluorobenzaldehyde (A113) (5.01 g, 21.8 mmol) in DMF (35 mL) was added Cs₂CO₃ (12.9 g, 39.5 mmol) and the reaction mixture was stirred at 50° C., while monitoring reaction progress by TLC. After complete consumption of starting material (3 h), the reaction mixture was cooled to room temperature and ice-cold water (30 mL) was added to it. Extraction was carried out using EtOAc (3×70 mL); the combined organic extracts were washed with brine solution (2×50 mL), dried over anhydrous Na₂SO₄, filtered and concentrated under reduced pressure. The residue was purified by Combi-Flash (using gradient elution of 0-50% of EtOAc in heptane) to afford desired compound 1-44 (3.3 g) as a pale-yellow liquid. ¹H NMR (400 MHz, DMSO-d₆): δ 10.28 (s, 1H); 7.72-7.69 (m, 2H); 7.49-7.47 (m, 2H); 7.41 (t, J=7.2 Hz, 2H); 7.36-7.33 (m, 1H); 6.97 (d, J=2.0 Hz, 1H); 6.66 (dd, J₁=2.0 Hz, J₂=8.4 Hz, 1H); 5.94 (d, J=2.0 Hz, 1H); 5.25 (s, 2H); 3.78 (s, 3H).

### Intermediate 45: Preparation of 1-(2,4-bis(benzyloxy)phenyl)ethan-1-amine (I-45)

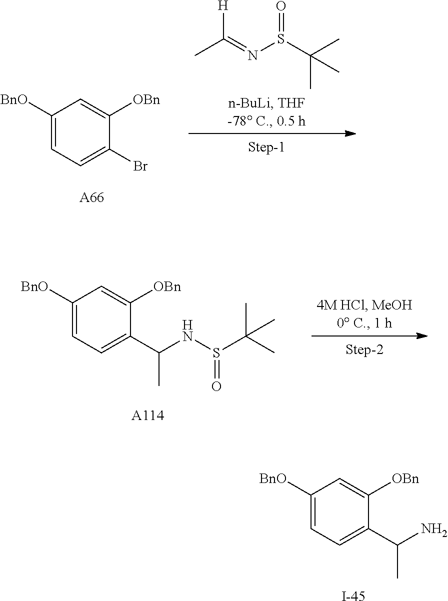

Step-1: Synthesis of N-{1-[2,4-bis(benzyloxy)phenyl]ethyl}-2-methylpropane-2-sulfinamide (A114): To a stirred solution of 2,4-bis(benzyloxy)-1-bromobenzene (A66) (10.0 g, 27.1 mmol) in THE (120 mL) was added n-BuLi (2.08 g, 32.5 mmol) at −78° C. and stirred for 30 min, before addition of a solution of N-[(E)-ethylidene]-2-methylpropane-2-sulfinamide (5.98 g, 40.6 mmol) in THE (10 mL). The reaction mixture was stirred at the same temperature, while reaction progress was monitored by TLC/LCMS analysis. After complete consumption of starting material (1 h), the reaction mixture was quenched with addition of saturated NH₄Cl solution (30 mL). Extraction was carried out using EtOAc (3×70 mL); the combined organic extracts were washed with brine solution (2×50 mL), dried over anhydrous Na₂SO₄, filtered and concentrated under reduced pressure. The residue was purified by Combi-Flash (using gradient elution of 0-50% of EtOAc in heptane) to afford desired compound A114 (8.00 g) as a thick oil. LCMS (ES) m/z: 438.2 [M+H]⁺.

Step-2: Synthesis of 1-[2,4-bis(benzyloxy)phenyl]ethan-1-amine (I-45): To a stirred solution of N-{1-[2,4-bis(benzyloxy)phenyl]ethyl}-2-methylpropane-2-sulfinamide (A114) (4.0 g, 9.14 mmol) in MeOH (40 mL) was added a 4 M solution of HCl in 1,4-dioxane (20 mL) at 0° C. The reaction mixture was stirred at 0° C., while monitoring reaction progress by TLC. After complete consumption of starting material (1 h), volatiles were removed under reduced pressure and saturated NaHCO₃ solution (30 mL) was added to the residue. Extraction was carried out using EtOAc (30 mL×2); the combined organic extracts were washed with brine (30 mL), dried over anhydrous Na₂SO₄, filtered and concentrated under reduced pressure to afford desired compound I-45 (1.75 g) as an off-white solid. LCMS (ES) m/z: 317.2 [(M-NH₃)+H]⁺.

### Intermediate 46: Preparation of 1-(N-(3-(aminomethyl)-2-fluorophenyl)sulfamoyl)pyrrolidine-3-carboxamide (I-46)

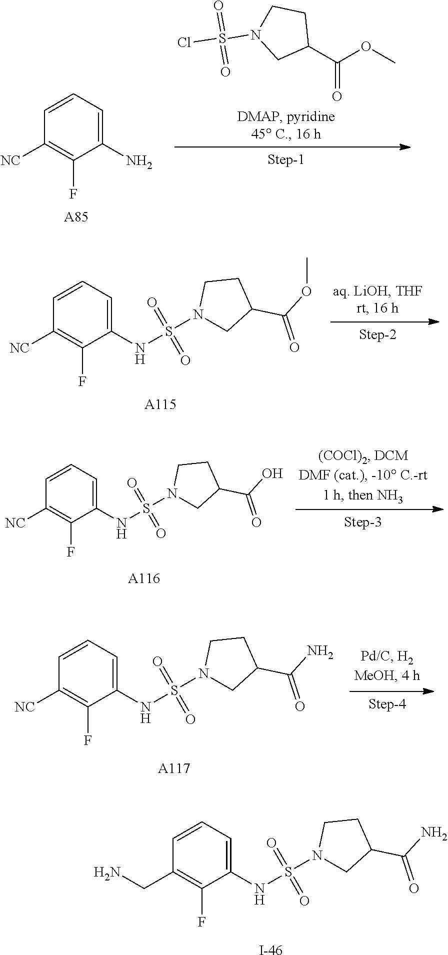

Step-1: Synthesis of methyl 1-[(3-cyano-2-fluorophenyl)sulfamoyl]pyrrolidine-3-carboxylate (A115): To a stirred solution of 3-amino-2-fluorobenzonitrile (A85) (2.69 g, 19.8 mmol) in pyridine (2 mL) was added DMAP (0.487 g, 3.95 mmol) and methyl 1-(chlorosulfonyl)pyrrolidine-3-carboxylate (4.5 g, 19.8 mmol) at room temperature. The reaction mixture was stirred at 45° C. and the reaction progress was monitored by TLC and LCMS analysis. After completion of the reaction (16 h), the reaction mixture was cooled to room temperature and then quenched with addition of water (30 mL). Extraction was carried out using EtOAc (3×70 mL); the combined organic extracts were washed with brine solution (2×50 mL), dried over anhydrous Na₂SO₄, filtered and concentrated under reduced pressure. The residue was purified by Combi-Flash (using gradient elution of 0-80% of EtOAc in heptane) to afford desired compound A115 (4.2 g) as brown solid. LCMS (ES) m/z: 328.1 [M+H]⁺.

Step-2: Synthesis of 1-[(3-cyano-2-fluorophenyl)sulfamoyl]pyrrolidine-3-carboxylic acid (A116): To a stirred solution of methyl 1-[(3-cyano-2-fluorophenyl)sulfamoyl]pyrrolidine-3-carboxylate (A115) (4.2 g, 12.8 mmol) in THE (88 mL) was added an aqueous solution of lithium hydroxide (1.03 g, 25.7 mmol, in 18 mL water) at room temperature. The reaction progress was monitored by TLC/LCMS analysis. After complete consumption of starting material (4 h), the reaction mixture was acidified using 1 M HCL (15 mL) and extraction was carried out using DCM (3×70 mL). The combined organic extracts were washed with brine solution (2×50 mL), dried over anhydrous Na₂SO₄, filtered and concentrated under reduced pressure. The residue was purified by Combi-Flash (using gradient elution of 0-5% of MeOH in DCM) to afford desired compound A116 (2.7 g) as a brown solid. LCMS (ES) m/z: 312.1 [M−H]⁺.

Step-3: Synthesis of 1-[(3-cyano-2-fluorophenyl)sulfamoyl]pyrrolidine-3-carboxamide (A117): To a stirred solution of 1-[(3-cyano-2-fluorophenyl)sulfamoyl]pyrrolidine-3-carboxylic acid (A116) (2.7 g, 8.62 mmol) in DCM (50 mL) was added oxalylchloride (1.9 mL, 21.6 mmol) drop-wise under N2 atmosphere followed by addition of 5 drops of DMF at room temperature. After stirring for 1 h, the reaction mixture was cooled to −10° C. and ammonia gas was purged through it for 20 min. The reaction mixture was then stirred for additional 1 h. Water (15 mL) was then added to the reaction mixture and extraction was carried out using DCM (3×70 mL). The combined organic extracts were washed with brine solution (2×50 mL), dried over anhydrous Na₂SO₄, filtered and concentrated under reduced pressure. The residue was purified by Combi-Flash (using gradient elution of 0-10% of MeOH in DCM) to afford desired compound A117 (2.23 g) as an off-white solid. LCMS (ES) m/z: 313.0 [M+H]⁺.

Step-4: Synthesis of 1-{[3-(aminomethyl)-2-fluorophenyl]sulfamoyl}pyrrolidine-3-carboxamide (I-46): To a stirred solution of 1-[(3-cyano-2-fluorophenyl)sulfamoyl]pyrrolidine-3-carboxamide (A117) (2.23 g, 7.14 mmol) in MeOH (60 mL) was added 10% Pd/C (3.04 g) and the reaction mixture was stirred under hydrogen atmosphere (20 psi pressure) for 6 h at 40° C. The reaction progress was monitored by TLC/LCMS analysis. After complete consumption of starting material (6 h), the catalyst was filtered off through a celite pad and washed with MeOH (5 mL×2). MeOH was then evaporated under reduced pressure to afford the desired compound I-46 (1.20 g) as a pale yellow semi-solid. LCMS (ES) m/z: 315.2 [M−H]⁺.

### Intermediate 47: Preparation of 3-(3-(aminomethyl)-2-fluorophenyl)oxazolidin-2-one (I-47)

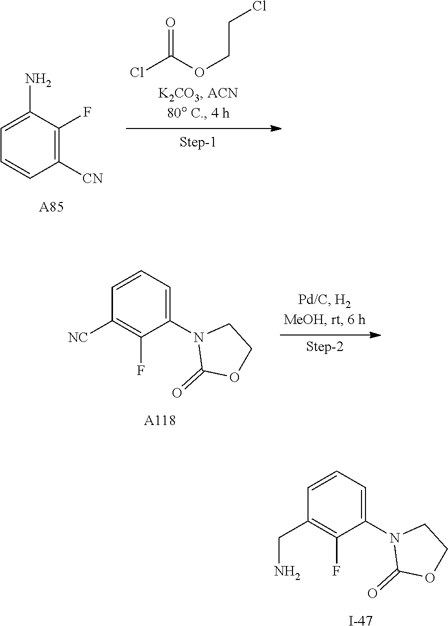

Step-1: Synthesis of 2-fluoro-3-(2-oxo-1,3-oxazolidin-3-yl)benzonitrile (A118): To a stirred solution of 3-amino-2-fluorobenzonitrile (A85) (8 g, 58.8 mmol) in acetonitrile (80 mL) was added K₂CO₃ (24.4 g, 176 mmol) and 2-chloroethyl carbonochloridate (6.07 mL, 58.8 mmol) at room temperature. The reaction mixture was stirred at 80° C. and the reaction progress was monitored by TLC/LCMS analysis. After complete consumption of starting material (4 h), the reaction mixture was cooled to room temperature and then quenched with water (30 mL). Extraction was carried out using EtOAc (3×70 mL); the combined organic extracts were washed with brine solution (2×50 mL), dried over anhydrous Na₂SO₄, filtered and concentrated under reduced pressure. The residue was purified by Combi-Flash (using gradient elution of 0-50% of EtOAc in heptane) to afford desired compound A118 (6 g) as an off-white solid. ¹H NMR (400 MHz, DMSO-d₆): δ 7.95 (t, J=7.6 Hz, 1H); 7.84 (t, J=7.6 Hz, 1H); 7.64 (t, J=8.0 Hz, 1H); 4.50 (t, J=7.2 Hz, 2H); 4.07 (t, J=8.0 Hz, 2H).

Step-2: Synthesis of 3-[3-(aminomethyl)-2-fluorophenyl]-1,3-oxazolidin-2-one (I-47): Intermediate 1-47 (3.45 g) was synthesized by following procedure as described for the synthesis of I-46 (step-4) using 2-fluoro-3-(2-oxo-1,3-oxazolidin-3-yl)benzonitrile (A118) (4 g, 19.4 mmol) as the starting material. LCMS (ES) m/z: 211.2 [M+H]⁺.

### Intermediate 48: Preparation of 3-(3-(aminomethyl)-2-fluorobenzyl)oxazolidin-2-one (I-48)

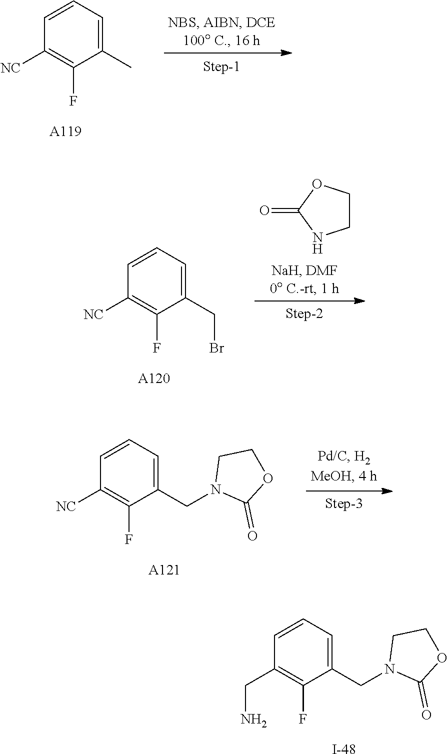

Step-1: Synthesis of 3-(bromomethyl)-2-fluorobenzonitrile (A120): To a stirred solution of 2-fluoro-3-toluonitrile (A119) (5.0 g, 37 mmol) in DCE (30 mL) was added AIBN (0.608 g, 3.7 mmol) and NBS (6.59 g, 37 mmol) at room temperature. The reaction mixture was stirred at 80° C., while monitoring reaction progress by TLC/LCMS analysis. After completion of the reaction (16 h), the reaction mixture was cooled to room temperature and quenched with addition of ice-cold water (50 mL). Extraction was carried out using EtOAc (3×70 mL); the combined organic extracts were washed with brine solution (2×50 mL), dried over anhydrous Na₂SO₄, filtered and concentrated under reduced pressure. The residue was purified by Combi-Flash (using gradient elution of 0-20% of EtOAc in heptane) to afford desired compound A120 (4.2 g) as a colorless liquid. ¹H NMR (400 MHz, DMSO-d₆): 37.95-7.92 (m, 2H); 7.45-7.42 (m, 1H); 4.76 (s, 2H).

Step-2: Synthesis of 2-fluoro-3-[(2-oxo-1,3-oxazolidin-3-yl)methyl]benzonitrile (A121): To a stirred solution of 1,3-oxazolidin-2-one (3.16 g, 36.3 mmol) in THE (60 mL) was added sodium hydride (60% dispersion in mineral oil) (1.52 g, 38.1 mmol) portion wise at 0° C. and stirred at 0° C. for 15 min. To this was then added a solution of 3-(bromomethyl)-2-fluorobenzonitrile (A120) (3.7 g, 17.3 mmol, in 30 mL THF) dropwise. The reaction mixture was then allowed to stir at room temperature and the reaction progress was monitored by TLC/LCMS analysis. After completion of the reaction (1 h), the reaction mixture was quenched with addition of ice-cold water (20 ml) at 0° C. and extraction was carried out using EtOAc (3×70 mL). The combined organic extracts were washed with brine solution (2×50 mL), dried over anhydrous Na₂SO₄, filtered and concentrated under reduced pressure to afford desired compound A121 (2.50 g) as a white solid, which is carried forward without any purification. LCMS (ES) m/z: 221.2 [M+H]⁺.

Step-3: Synthesis of ({2-fluoro-3-[(2-oxo-1,3-oxazolidin-3-yl)methyl]phenyl}methyl)amine (I-48): Intermediate 1-48 (2.2 g) was synthesized by following procedure as described for the synthesis of 1-46 (step-4) using 2-fluoro-3-[(2-oxo-1,3-oxazolidin-3-yl)methyl]benzonitrile (A121) (2.5 g, 11.4 mmol) as the starting material. LCMS (ES) m/z: 225.1 [M+H]⁺.

### Intermediate 49: Preparation of 1-(3-(aminomethyl)-2-fluorobenzyl)imidazolidin-2-one (1-49)

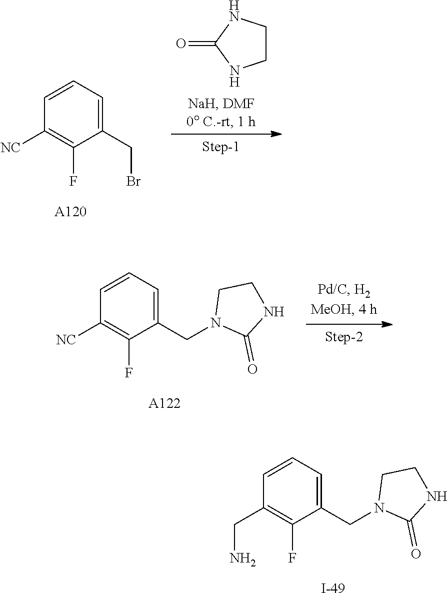

Step-1: Synthesis of2-fluoro-3-[(2-oxo-1-imidazolidinyl)methyl]benzonitrile (A122): Compound A122 (1.0 g) was synthesized by following procedure as described for the synthesis of I-48 (step-2) using 2-imidazolidinone (4.83 g, 56.1 mmol) and 3-(bromomethyl)-2-fluorobenzonitrile (A120) (3 g, 14 mmol) as the starting materials. LCMS (ES) m/z: 220.3 [M+H]⁺.

Step-2: Synthesis of ({2-fluoro-3-[(2-oxo-1-imidazolidinyl)methyl]phenyl}methyl)amine (I-49): Intermediate I-49 (0.83 g) was synthesized by following procedure as described for the synthesis of I-46 (step-4) using 2-fluoro-3-[(2-oxo-1-imidazolidinyl)methyl]benzonitrile (A122) (1 g, 4.56 mmol) as the starting material. LCMS (ES) m/z: 224.1 [M+H]⁺.

### Intermediate 50: Preparation of 4-(3-(aminomethyl)-2-fluorobenzyl)morpholin-3-one (I-50)

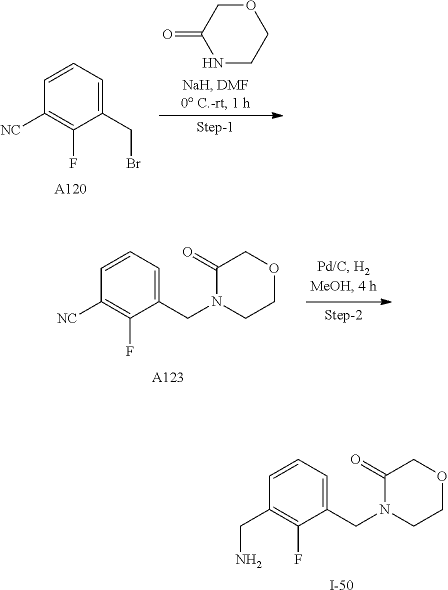

Step-1: Synthesis of 2-fluoro-3-[(3-oxo-4-morpholinyl)methyl]benzonitrile (A123): Compound A123 (9.5 g) was synthesized by following procedure as described for the synthesis of 1-48 (step-2) using 3-(bromomethyl)-2-fluorobenzonitrile (A120) (11 g, 51.4 mmol) and 3-morpholinone (10.9 g, 108 mmol) as the starting materials. LCMS (ES) m/z: 235.3 [M+H]⁺.

Step-2: Synthesis of 4-{[3-(aminomethyl)-2-fluorophenyl]methyl}-3-morpholinone (1-50): Intermediate 1-50 (3.5 g) was synthesized by following procedure as described for the synthesis of 1-46 (step-4) using 2-fluoro-3-[(2-oxo-1,3-oxazolidin-3-yl)methyl]benzonitrile (A123) (8.5 g, 36.3 mmol) as the starting material. LCMS (ES) m/z: 239.2 [M+H]⁺.

### Intermediate 51: Preparation of 2-hydroxy-4-(pyridin-3-yloxy)benzaldehyde (1-51)

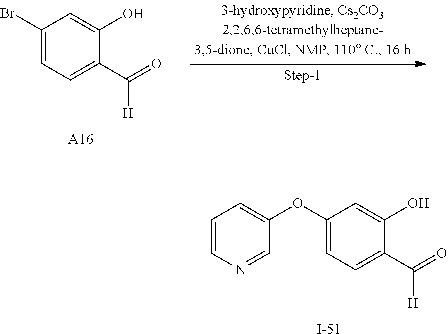

Step-1: Synthesis of 2-hydroxy-4-(pyridin-3-yloxy)benzaldehyde (I-51): To a stirred solution of 4-bromo-2-hydroxybenzaldehyde (A16) (1 g, 4.97 mmol) in NMP (10 mL) was added Cs₂CO₃ (3.24 g, 9.95 mmol), pyridin-3-ol (946 mg, 9.95 mmol), CuCl (246 mg, 2.49 mmol) and 2,2,6,6-tetramethylheptane-3,5-dione (229 mg, 1.24 mmol) at room temperature. The reaction mixture was stirred at 110° C. for 16 h. After completion of starting material, the reaction mixture was cooled to room temperature and then ice cold water (30 mL) was added to it. Extraction was carried out using EtOAc (30 mL×3); the combined organic extracts were washed with water (50 mL), brine (50 mL), dried over anhydrous Na₂SO₄, filtered and concentrated under reduced pressure. The residue was purified by Combi-Flash (using gradient elution of 0-10% of EtOAc in heptane) to afford desired compound I-51 (0.2 g) as a pale yellow solid. LCMS (ES) m/z: 216.1 [M+H]⁺.

### Intermediate 52: Preparation of 4-formyl-3-hydroxy-N,N-dimethylbenzamide (I-52)

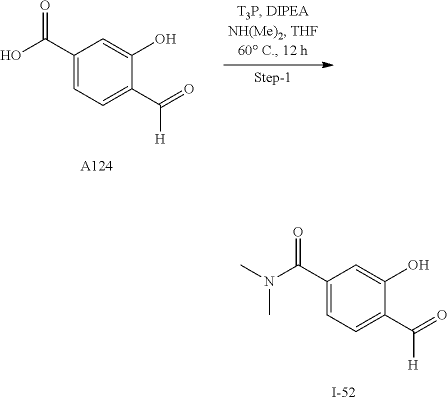

Step-1: Synthesis of 4-formyl-3-hydroxy-N-(propan-2-yl)benzamide (I-52): To a stirred solution of 4-formyl-3-hydroxybenzoic acid (A124) (1 g, 6.02 mmol) and dimethylamine (0.271 g, 6.02 mmol) in THE (10 mL) was added DIPEA (3.1 mL, 18 mmol) and a 50% solution of T₃P in EtOAc (7.66 mL, 12 mmol). The reaction mixture was stirred at 60° C. in a sealed tube for 12 h. After completion of the reaction, the reaction mixture was cooled to room temperature and quenched with addition of ice water (20 mL). Extraction was carried out using EtOAc (30 mL×3); the combined organic extracts were washed with water (50 mL), brine (50 mL), dried over anhydrous Na₂SO₄, filtered and concentrated under reduced pressure. The residue was purified by Combi-Flash (using gradient elution of 0-5% of MeOH in DCM) to afford desired compound I-52 (0.8 g) as a colorless semi-solid. ¹H NMR (400 MHz, DMSO-d₆): δ 10.93 (s, 1H); 10.29 (s, 1H); 7.68 (d, J=8.0 Hz, 1H); 6.95-6.77 (m, 2H); 2.97 (s, 3H); 2.87 (s, 3H).

### Intermediate 53: Preparation of 1-(2,4-bis(benzyloxy)-5-fluorophenyl)ethan-1-amine (I-53)

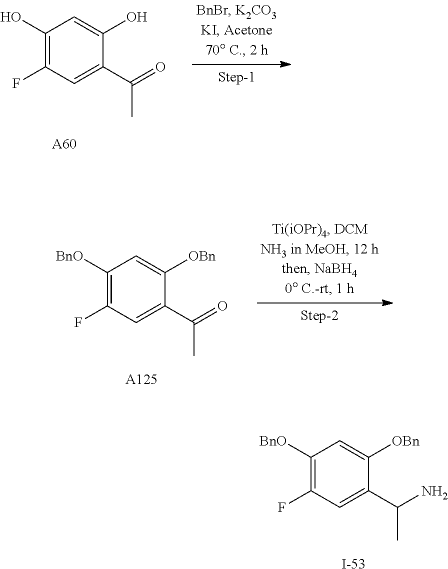

Step-1: Synthesis of 1-(2,4-bis(benzyloxy)-5-fluorophenyl)ethan-1-one (A125): To a stirred solution of 1-(5-fluoro-2,4-dihydroxyphenyl)ethan-1-one (A60) (15 g, 88.2 mmol) in acetone (150 mL) was added K₂CO₃ (36.6 g, 264 mmol), KI (29.3 g, 176 mmol) and benzyl bromide (26.2 mL, 220 mmol) at room temperature. The reaction mixture was stirred at 70° C. and the reaction progress was monitored by TLC/LCMS analysis. After completion of the reaction (2 h), the reaction mixture was cooled to room temperature and then water (100 mL) was added to it. Extraction was carried out using EtOAc (50 mL×3); the combined organic extracts were washed with water (50 mL), brine (50 mL), dried over anhydrous Na₂SO₄, filtered and concentrated under reduced pressure. The residue was purified by Combi-Flash (using gradient elution of 0-50% of EtOAc in heptane) to afford desired compound A125 (20 g) as an off-white solid. LCMS (ES) m/z: 351.2 [M+H]⁺.

Step 2: Synthesis of 1-(2,4-bis(benzyloxy)-5-fluorophenyl)ethan-1-amine (I-53): To a stirred suspension of 1-[2,4-bis(benzyloxy)-5-fluorophenyl]-1-ethanone (A125) (15 g, 42.8 mmol) in DCM (50 mL) was added a solution of ammonia in MeOH (6 mL) and titanium (IV) isopropoxide (24.3 g, 85.6 mmol). The reaction mixture was allowed to stir at room temperature for 12 h. The reaction mixture was then cooled to 0° C. and NaBH₄ (2.43 g, 64.2 mmol) was added to it. The reaction was slowly brought to room temperature and the reaction progress was monitored by TLC/LCMS analysis. After completion of the reaction (1 h), the reaction mixture was concentrated under reduced pressure and quenched with addition of water (50 mL). Extraction was carried out using EtOAc (50 mL×3); the combined organic extracts were washed with water (50 mL), brine (50 mL), dried over anhydrous Na₂SO₄, filtered and concentrated under reduced pressure. The residue was purified by Combi-Flash (using gradient elution of 0-50% of EtOAc in heptane) to afford desired compound I-53 (10 g) as an off-white solid. LCMS (ES) m/z: 352.1 [M+H]⁺.

Intermediate-55: Preparation of 4-(1-aminocyclobutyl)-5-fluorobenzene-1,3-diol (I-55)

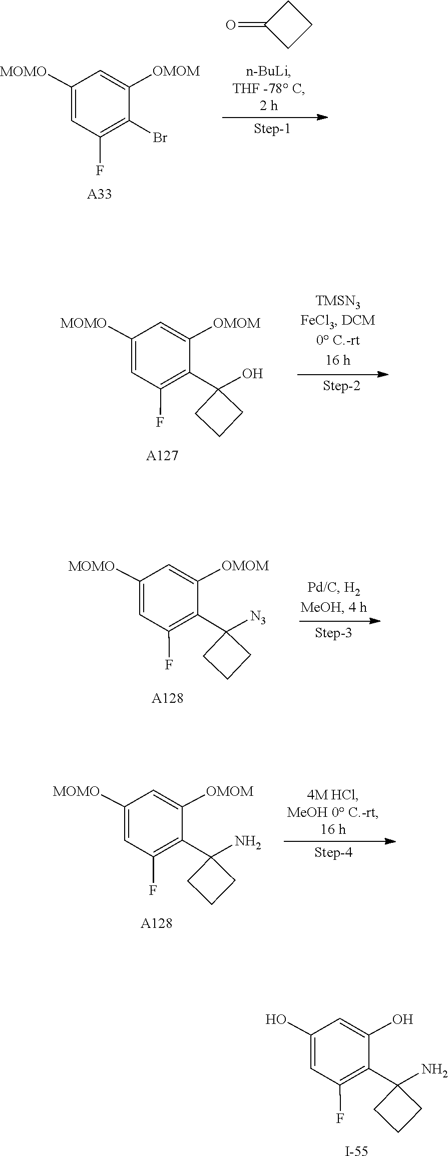

Step-1: Synthesis of 1-(2-fluoro-4,6-bis(methoxymethoxy)phenyl)cyclobutan-1-ol (127): Compound A127 (6.2 g) was synthesized by following procedure as described for the synthesis of I-30 (step-1) using A33 (10 g, 33.8 mmol) and cyclobutanone (7.13 g, 102 mmol) as starting materials. LCMS (ES) m/z: 269.1 [(M-OH)+H]⁺.

Step-2: Synthesis of 2-(1-azidocyclobutyl)-1-fluoro-3,5-bis(methoxymethoxy)benzene (A128): Compound A128 (6.5 g) was synthesized by following procedure as described for the synthesis of I-30 (step-2) using 1-(2-fluoro-4,6-bis(methoxymethoxy)phenyl)cyclobutan-1-ol (A127) (9.0 g, 31.4 mmol) as starting material. LCMS (ES) m/z: 269.1 [(M-N₃)+H]⁺.

Step-3: Synthesis of 1-(2-fluoro-4,6-bis(methoxymethoxy)phenyl)cyclobutan-1-amine (129): Compound A129 (5.2 g) was synthesized by following procedure as described for the synthesis of I-30 (step-3) using 2-(1-azidocyclobutyl)-1-fluoro-3,5-bis(methoxymethoxy)benzene (A128) (6.5 g) as starting material. LCMS (ES) m/z: 269.1 [(M-NH2)+H]⁺.

Step-4: Synthesis of 4-(1-aminocyclobutyl)-5-fluorobenzene-1,3-diol (I-55): Intermediate (I-55) (4.0 g) was synthesized by following procedure as described for the synthesis of I-39 (step-1) using 1-(2-fluoro-4,6-bis(methoxymethoxy)phenyl)cyclobutan-1-amine (A129) (5.2 g, 18.2 mmol) as starting material. LCMS (ES) m/z: 181.1 [(M-NH₂)+H]⁺.

### Intermediate-56: Preparation of 4-(1-aminocyclobutyl)-2-fluorobenzene-1,3-diol (I-56)

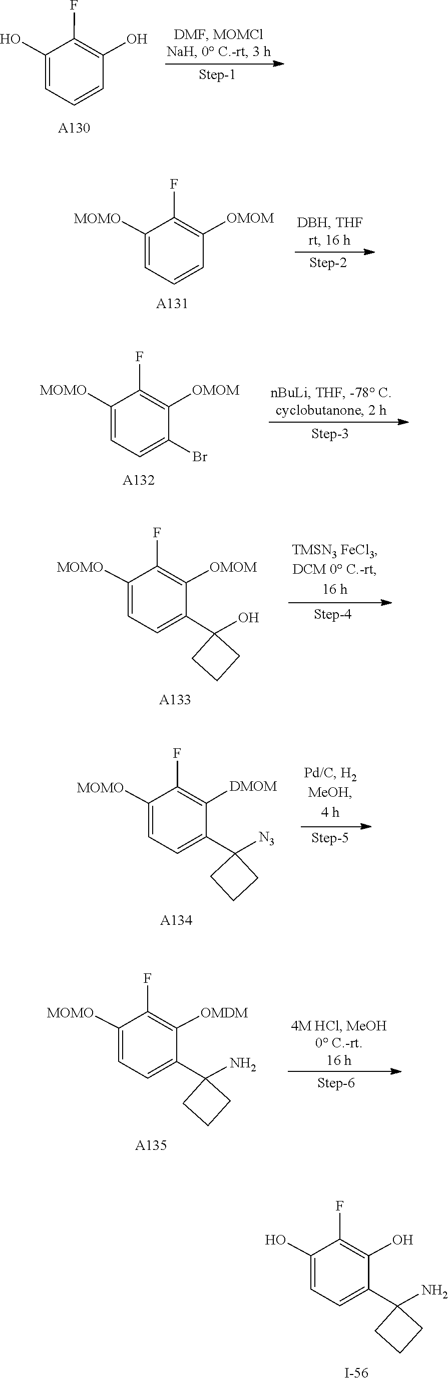

Step-1: Synthesis of 2-fluoro-1,3-bis(methoxymethoxy)benzene (A131): Compound A131 (31 g) was synthesized by following procedure as described for the synthesis of I-16 (step-1) using 2-fluorobenzene-1,3-diol (A130) (20 g, 156 mmol) as starting material. ¹H NMR (400 MHz, DMSO): δ 7.04-7.0 (m, 1H); 6.89 (t, J=7.6 Hz, 2H); 5.22 (s, 4H); 3.43 (s, 6H).

Step-2: Synthesis of 1-bromo-3-fluoro-2,4-bis(methoxymethoxy)benzene (A132): Compound A132 (25 g) was synthesized by following procedure as described for the synthesis of 1-26 (step-2) using 2-fluoro-1,3-bis(methoxymethoxy)benzene (A131) (31 g, 143.5 mmol) as starting material. ¹H NMR (400 MHz, DMSO): δ 7.40 (d, J=2.4 Hz, 1H); 7.03 (d, J=2.0 Hz, 1H); 5.25 (s, 2H); 5.15 (s, 2H); 3.79 (s, 3H); 3.29 (s, 3H).

Step-3: Synthesis of 1-(3-fluoro-2,4-bis(methoxymethoxy)phenyl)cyclobutan-1-ol (133): Compound A133 (15 g) was synthesized by following procedure as described for the synthesis of 1-30 (step-1) using 1-bromo-3-fluoro-2,4-bis(methoxymethoxy)benzene (A132) (25 g, 85.3 mmol) as starting material. ¹H NMR (400 MHz, DMSO): h 7.02-6.93 (m, 2H); 5.29 (s, 1H); 5.21-5.13 (m, 4H); 3.50 (s, 3H); 3.40 (s, 3H); 2.26-2.12 (m, 2H); 2.03-1.89 (m, 4H).

Step-4: Synthesis of 1-(1-azidocyclobutyl)-3-fluoro-2,4-bis(methoxymethoxy)benzene (A134): Compound A134 (10 g) was synthesized by following procedure as described for the synthesis of 1-30 (step-2) using 1-(3-fluoro-2,4-bis(methoxymethoxy)phenyl)cyclobutan-1-ol (A133) (15 g, 52.4 mmol) as starting material.

¹H NMR (400 MHz, CDCl₃): δ 6.86-6.77 (m, 2H); 5.15-5.13 (m, 4H); 3.56 (s, 3H); 3.50 (s, 3H); 2.64-2.45 (m, 2H); 2.44-2.39 (m, 2H); 2.23-2.11 (m, 1H); 1.80-1.73 (m, 1H).

Step-5: Synthesis of 1-(3-fluoro-2,4-bis(methoxymethoxy)phenyl)cyclobutan-1-amine (A135): Intermediate A135 (7 g) was synthesized by following procedure as described for the synthesis of 1-30 (step-3) using 1-(1-azidocyclobutyl)-3-fluoro-2,4-bis(methoxymethoxy)benzene (A134) (10 g, 32.1 mmol) as the starting material. ¹H NMR (400 MHz, DMSO): δ 6.92-6.87 (m, 2H); 5.20-5.17 (m, 4H); 3.51 (s, 2H); 3.40 (s, 6H); 2.49-2.37 (m, 2H); 2.10-2.06 (m, 2H); 1.91-1.90 (m, 2H).

Step-6: Synthesis of 4-(1-aminocyclobutyl)-2-fluorobenzene-1,3-diol (I-56): Intermediate I-56 (4.5 g) was synthesized by following procedure as described for the synthesis of 1-39 (step-1) using 1-(3-fluoro-2,4-bis(methoxymethoxy)phenyl)cyclobutan-1-amine (A135) (7 g, 24.5 mmol) as starting material. ¹H NMR (400 MHz, DMSO): δ 10.13 (s, 1H); 9.97 (s, 1H); 8.17 (s, 2H); 6.80 (d, J=8.8 Hz, 1H); 6.42-6.38 (m, 1H); 2.51-2.42 (m, 2H); 2.11-2.00 (m, 2H); 1.68-1.60 (m, 2H).

### Intermediate-57: Preparation of 2-(dimethylaminosulfonylamino)-3-fluoroisonicotinaldehyde (1-57)

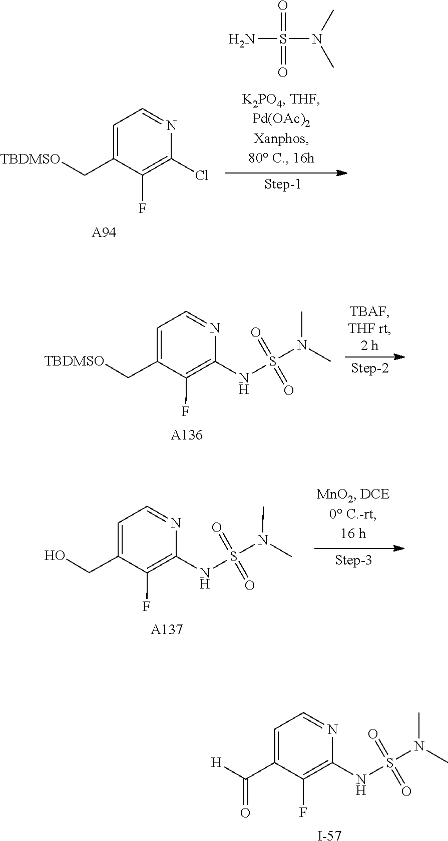

Step-1: Synthesis of 4-{[(tert-butyl)bis(methyl)siloxy]methyl}-2-(dimethylaminosulfonylamino)-3-fluoropyridine (A136): Compound A136 (4.2 g) was synthesized by following procedure as described for the synthesis of 1-35 (step-3) using (tert-butyl)[(2-chloro-3-fluoro-4-pyridyl)methoxy]bis(methyl)silane (A94) (10 g, 36.3 mmol) and N,N-dimethylaminosulfonylamine (18 g, 145 mmol) as starting materials. LCMS (ES) m/z: 364.1 [M+1]⁺.

Step-2: Synthesis of [2-(dimethylaminosulfonylamino)-3-fluoro-4-pyridyl]methanol (A137): Compound A137 (3.7 g, crude) was synthesized by following procedure as described for the synthesis of 1-35 (step-4) using 4-{[(tert-butyl)bis(methyl)siloxy]methyl}-2-(dimethylaminosulfonylamino)-3-fluoropyridine (A136) (4.2 g, 11.6 mmol) as starting material. LCMS (ES) m/z: 250.1 [M+H]⁺.

Step-3: Synthesis of 2-(dimethylaminosulfonylamino)-3-fluoroisonicotinaldehyde (1-57): Intermediate 1-57 (0.95 g) was synthesized by following procedure as described for the synthesis of 1-35 (step-5) using [2-(dimethylaminosulfonylamino)-3-fluoro-4-pyridyl]methanol (A137) as starting material (3.1 g, 12.4 mmol). LCMS (ES) m/z: 248.1 [M+1]⁺.

### Intermediate-58: Preparation of 1-(2,4-bis(benzyloxy)-5-fluorophenyl)ethan-1-amine (I-58)

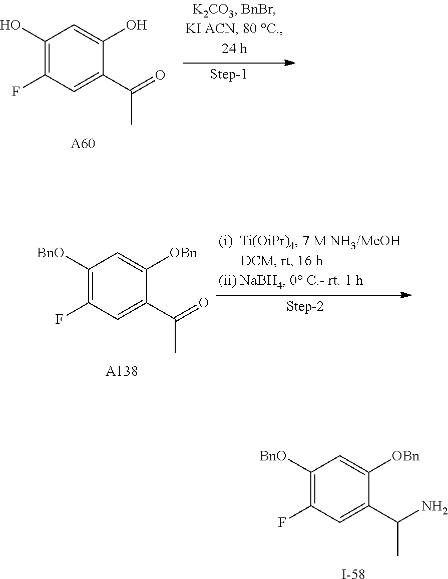

Step-1: Synthesis of 1-(2,4-bis(benzyloxy)-5-fluorophenyl)ethan-1-amine (A136): Compound A138 (27 g) was synthesized by following procedure as described for the synthesis of 1-26 (step-1) using 1-(5-fluoro-2,4-dihydroxyphenyl)ethan-1-one (A60) (20 g, 118 mmol) as starting material. LCMS (ESI) m/z: 351.10 [M+H]⁺.

Step-2: Synthesis of 1-(2,4-bis(benzyloxy)-5-fluorophenyl)ethan-1-amine (I-58): To a stirred solution of 1-(2,4-bis(benzyloxy)-5-fluorophenyl)ethan-1-one (A138) (13.0 g, 37.1 mmol) in DCM (100 mL) was added a 7N solution of ammonia in MeOH (8 mL) followed by titanium (IV) isopropoxide (22.5 mL, 74.2 mmol). The reaction mixture was allowed to stir at room temperature for 16 h. Then, the reaction mixture was cooled to 0° C. and NaBH₄ (2.11 g, 55.7 mmol) was added to it. The reaction mixture was stirred for 1 h at room temperature. The reaction progress was monitored by TLC/LCMS. After completion of the reaction (1 h), the reaction mixture was quenched with addition of ice water (30 mL) and extraction was carried out using EtOAc (3×30 mL). The combined organic extracts were washed with brine solution (30 mL), dried over anhydrous Na₂SO₄, filtered and evaporated under reduced pressure. The residue was purified by Combi-Flash (using gradient elution of 0-10% of MeOH in DCM) to afford the desired intermediate 1-58 (8.0 g) as a brown oil. LCMS (ESI) m/z: 335.15 [M-NH₂]⁺.

### Intermediate-59: Preparation of 1-(3,5-bis(benzyloxy)pyridin-2-yl)ethan-1-amine (I-59)

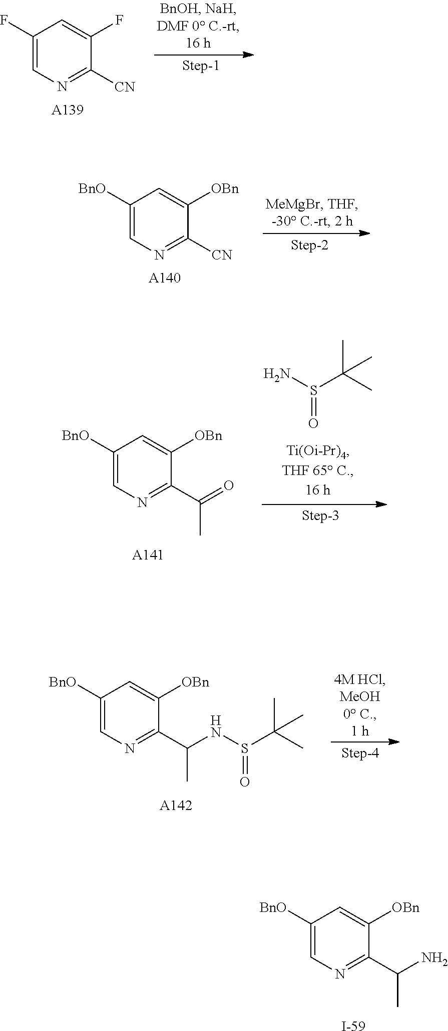

Step-1: Synthesis of 3,5-bis(benzyloxy)picolinonitrile (A140): To a stirred solution of benzyl alcohol (74.2 mL, 714 mmol) in DMF (0.5 L, 6.46 mol) was added sodium hydride (60% in mineral oil, 17.1 g, 714 mmol) at 0° C. and the reaction mixture was stirred at room temperature for 1 h. It was then cooled to 0° C. and 3,5-difluoro-2-pyridinecarbonitrile (A139) (50 g, 357 mmol) was added to it. After stirring at room temperature for 16 h, the reaction was quenched with addition of water (400 mL). The precipitate obtained was collected by vacuum filtration to afford the desired compound A140 (45.0 g) as a white solid. ¹H NMR (400 MHz, DMSO-d₆): δ 8.13 (d, J=2 Hz, 1H); 7.56 (d, J=2 Hz, 1H); 7.48-7.30 (m, 10H); 5.35 (s, 2H); 5.30 (s, 2H).

Step-2: Synthesis of 1-(3,5-bis(benzyloxy)pyridin-2-yl)ethan-1-one (A141): To a stirred solution of 3,5-bis(benzyloxy)picolinonitrile (A140) (22.0 g, 69.5 mmol) in anhydrous THE (161 mL) was added a solution of MeMgBr (3.0 M in diethyl ether, 46.4 mL, 139 mmol) dropwise at −30° C. The reaction mixture was then stirred at room temperature for 2 h under nitrogen atmosphere. The reaction progress was monitored by TLC. After complete consumption of starting material (2 h), the reaction mixture was quenched with addition of 2N HCl and then diluted with water (10 mL). Extraction was carried out using EtOAc (3×100 mL). The combined organic extracts were washed with brine (100 mL), dried over Na₂SO₄, filtered and concentrated under reduced pressure. The residue was purified by Combi-Flash (using gradient elution of 0-30% EtOAc in heptane) to afford the desired compound (A141) (15.0 g) as a white solid. ¹H NMR (400 MHz, DMSO-d₆): δ 8.04 (d, J=2.4 Hz, 1H); 7.45 (m, 2H), 7.40-7.30 (m, 8H); 6.90 (d, J=2.0 Hz, 1H); 5.19 (s, 2H), 5.12 (s, 2H); 2.64 (s, 3H).

Step-3: Synthesis of N-(1-(3,5-bis(benzyloxy)pyridin-2-yl)ethyl)-2-methylpropane-2-sulfinamide (A142): To a stirred solution of 1-(3,5-bis(benzyloxy)pyridin-2-yl)ethan-1-one (A141) (15 g, 45 mmol) in THE (0.3 L, 3.69 mol) was added 2-methylpropane-2-sulfinamide (5.45 g, 45 mmol) and Ti(OⁱPr)₄ (20 mL, 67.5 mmol) at room temperature. The reaction mixture was allowed to stir at 65° C. for 16 h. The reaction progress was monitored by TLC. After complete consumption of starting material, the reaction mixture was cooled to 0° C. and sodium borohydride (1.7 g, 45 mmol) was added portion wise. The reaction mixture was stirred at room temperature, while monitoring reaction by TLC. After complete consumption of starting material, the reaction mixture was quenched with water (50 mL) and filtered through celite. The filtrate was concentrated under reduced pressure and the residue was purified by Combi-Flash (using gradient elution of 0-35% of EtOAc in heptane) to afford the desired compound A142 (5.2 g). LCMS (ES) nm/z: 439.20 [M+1]⁺.

Step-4: Synthesis of 1-(3,5-bis(benzyloxy)pyridin-2-yl)ethan-1-amine (I-59): To a stirred solution of N-(1-(3,5-bis(benzyloxy)pyridin-2-yl)ethyl)-2-methylpropane-2-sulfinamide (A142) (5.2 g, 11.9 mmol) in MeOH (52 mL) was added a 4 M solution of HCl in 1,4-dioxane (7.4 mL, 29.6 mmol) at 0° C. and stirred at the same temperature for 1 h. Reaction was monitored by TLC and LCMS. After completion of the reaction, volatiles were removed under reduced pressure and saturated NaHCO₃ solution (30 mL) was added to the residue. Extraction was carried out using EtOAc (50 mL×3), the combined organic extracts were washed with brine solution, dried over Na₂SO₄, filtered and concentrated under reduced pressure to afford desired compound (I-59) (3.0 g) as light brown semi solid. LCMS (ES) m/z: 335.30 [M+H]⁺.

### Intermediate-60: Preparation of tert-butyl (3-(bromomethyl)-2-fluorophenyl)carbamate (I-60)

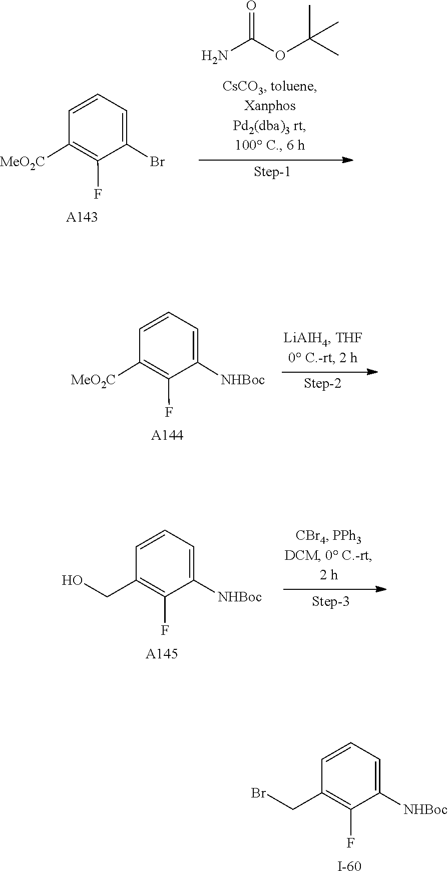

Step-1: Synthesis of methyl 3-((tert-butoxycarbonyl)amino)-2-fluorobenzoate (A144): Argon gas was purged through a stirred suspension of methyl 3-bromo-2-fluorobenzoate (A143) (50 g, 215 mmol), caesium carbonate (83.9 g, 257 mmol) and tert-butyl carbamate (30.2 g, 257 mmol) in toluene (0.5 L) for 10 min, before addition of Xantphos (7.45 g, 12.9 mmol) and Pd₂(dba)₃ (4.91 g, 5.36 mmol). The reaction mixture was allowed to stir at 100° C. for 6 h. The reaction progress was monitored by TLC/LCMS. After completion of the reaction (6 h), the reaction mixture was cooled to room temperature and water (100 mL) was added to it. Extraction was carried out using EtOAc (3×3000 mL), the combined organic extracts were washed with brine solution (100 mL), dried over anhydrous Na₂SO₄, filtered and concentrated under reduced pressure. The crude was purified by Combi-Flash (using gradient elution of 0-40% EtOAc in heptane) to afford the desired compound A144 (42.0 g) as a yellow solid. LCMS (ES) m/z: 268.15 [M−H]⁺.

Step-2: Synthesis of tert-butyl (2-fluoro-3-(hydroxymethyl)phenyl)carbamate (A145): To a stirred solution of methyl 3-((tert-butoxycarbonyl)amino)-2-fluorobenzoate (A144) (42 g, 156 mmol) in anhydrous THF (420 mL) was added a 2 M solution of LAH in THF (156 mL, 312 mmol) slowly at 0° C. Then the reaction mixture was stirred at room temperature for 2 h, while monitoring reaction progress by TLC/LCMS. After completion, the reaction mixture was quenched with addition of saturated NH₄Cl solution (50 mL) and extracted with EtOAc (3×100 mL). The combined organic extracts were washed with brine solution (100 mL), dried over anhydrous Na₂SO₄, filtered and evaporated the solvent under reduced pressure. The residue was purified by Combi-Flash (using gradient elution of 0-50% of EtOAc in heptane) to afford desired compound A145 (32.0 g) as an off-white solid. ¹H NMR (400 MHz, DMSO-d₆): δ 8.89 (brs, 1H); 7.48-7.44 (m, 1H); 7.17-7.06 (m, 2H); 5.25 (t, J=5.6 Hz, 1H); 4.52 (d, J=5.6 Hz, 3H); 1.45 (s, 9H).

Step-3: Synthesis of tert-butyl (3-(bromomethyl)-2-fluorophenyl)carbamate (I-60): To a stirred solution of tert-butyl (2-fluoro-3-(hydroxymethyl)phenyl)carbamate (A145) (32.0 g, 133 mmol) in DCM (380 mL) was added PPh₃ (52.2 g, 199 mmol) and CBr₄ (66 g, 199 mmol) at 0° C. The reaction mixture was then allowed to stir at room temperature for 2 h, while monitoring reaction progress by TLC/LCMS. After completion of the reaction, ice water (100 mL) was added to it and extracted with DCM (2×500 mL). The combined organic extracts were washed with brine solution (100 mL), dried over anhydrous Na₂SO₄, filtered and concentrated under reduced pressure. The residue was purified by Combi-Flash (using gradient elution of 0-30% of EtOAc in heptane) to afford the desired compound I-60 (15.0 g) as a white solid. LCMS (ES) m/z: 302.10 |M−H|⁺. ¹H NMR (400 MHz, DMSO-d₆): δ 9.16 (s, 1H); 7.69 (t, J=7.6 Hz, 1H); 7.31-7.27 (m, 1H); 7.10 7.69 (t, J=8.0 Hz, 1H); 4.68 (s, 2H); 1.45 (s, 9H).

### Intermediate-61: Preparation of tert-butyl (tert-butoxycarbonyl)(2,5-difluoro-3-formylphenyl)carbamate (I-61)

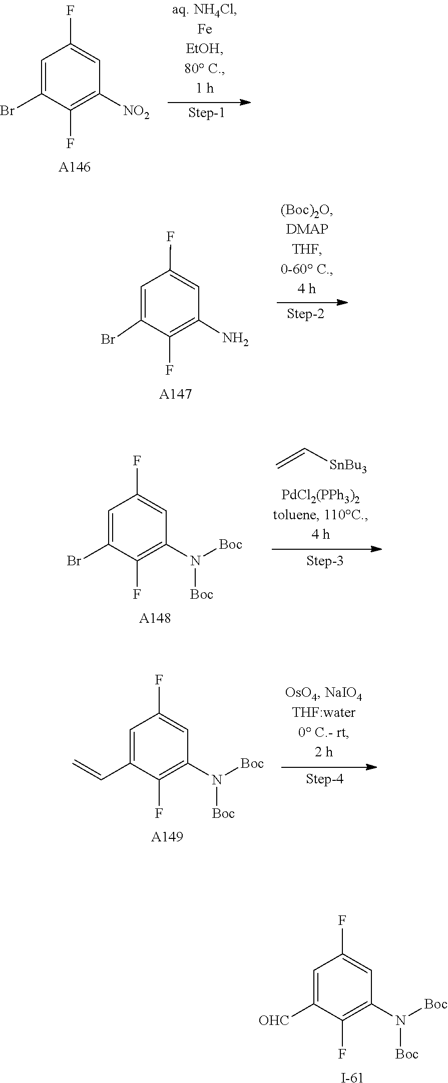

Step-1: Synthesis of 3-bromo-2,5-difluoroaniline (A147): To a stirred solution of 1-bromo-2,5-difluoro-3-nitrobenzene (A146) (15.0 g, 63 mmol) in EtOH (60 mL) was added iron (16.5 g, 295 mmol) and an aqueous solution of ammonium chloride (22.5 g, 421 mmol, in 20 mL water); and reaction mixture was stirred at 80° C. for 1 h. After complete consumption of starting material (1 h), the reaction mixture was cooled to room temperature and water (20 mL) was added to it. Extraction was carried out using EtOAc (2×30 mL); the combined organic extracts were dried over anhydrous sodium sulphate, filtered and concentrated under reduced pressure to afford desired compound A147 (11.0 g) as a brown sticky liquid. ¹H NMR (400 MHz, DMSO-d₆): δ 6.64-6.60 (m, 1H); 6.56-6.51 (m, 1H); 5.79 (s, 2H).

Step-2: Synthesis of tert-butyl (3-bromo-2,5-difluorophenyl) (tert-butoxycarbonyl)carbamate (A148): To a stirred solution of 3-bromo-2,5-difluoroaniline (A147) (10 g, 48.1 mmol) in THE (100 mL) was added DMAP (0.587 g, 4.81 mmol) and (Boc)₂₀ (33.1 mL, 144 mmol) at 0° C.; and then the reaction mixture was allowed to stir at 60° C. for 4 h. The reaction progress was monitored by TLC. After completion of the reaction, the reaction mixture was cooled to room temperature and water (40 mL) was added to it. Extraction was carries out using EtOAc (2×45 mL); the combined organic extracts were washed with brine solution (40 mL), dried over anhydrous Na₂SO₄, filtered and concentrated under reduced pressure. The residue was purified by Combi-Flash (using gradient elution of 0-20% of EtOAc in heptane) to afford desired compound A148 (16.0 g) as a brown solid. ¹H NMR (400 MHz, DMSO-d₆): δ 7.81-7.77 (m, 1H); 7.61-7.56 (m, 1H); 1.38 (s, 18H).

Step-3: Synthesis of tert-butyl (tert-butoxycarbonyl)(2,5-difluoro-3-vinylphenyl)carbamate (A149): Argon gas was purged through a stirred solution of tert-butyl (3-bromo-2,5-difluorophenyl)(tert-butoxycarbonyl)carbamate (A148) (16.0 g, 39.2 mmol) and tributyl(vinyl)stannane (12.4 g, 39.2 mmol) in toluene (0.1 L) for 10 min before addition of PdCl₂(PPh₃)₂ (2.75 g, 3.92 mmol). The reaction mixture was stirred at 110° C. for 4 h, before it was cooled to room temperature. Water (40 mL) was then added to it and extraction was carried out using EtOAc (3×40 mL). The combined organic extracts were washed with brine (40 mL), dried over anhydrous sodium sulfate, filtered and concentrated under reduced pressure. The residue was purified by Combi-Flash (using gradient elution of 0-10% of MeOH in DCM) to afford desired compound A149 (12.5 g) as a yellow solid. ¹H NMR (400 MHz, DMSO-d₆): δ 7.60-7.56 (m, 1H); 7.38-7.34 (m, 1H); 6.81 (dd, J=17.6, 11.2 Hz, 1H); 6.04 (d, J=17.6 Hz, 1H); 5.53 (d, J=11.2 Hz, 1H); 1.38 (s, 18H).

Step-4: Synthesis of tert-butyl (tert-butoxycarbonyl)(2,5-difluoro-3-formylphenyl)carbamate (I-61): To a stirred solution of tert-butyl (tert-butoxycarbonyl)(2,5-difluoro-3-vinylphenyl)carbamate (A149) (5.0 g, 14.1 mmol) in tetrahydrofuran (40 mL) and water (20 mL) was added OSO₄ (1.79 g, 7.03 mmol) at 0° C. and the reaction mixture was allowed to stir at room temperature for 10 min. Then sodium periodate (7.52 g, 35.2 mmol) was added to it at the same temperature and the reaction mixture was stirred at room temperature while monitoring reaction progress. After completion of the reaction (2 h), water (20 mL) was added to it and extraction was carried out using EtOAc (2×35 mL). The combined organic extracts were washed with brine solution (40 mL), dried over anhydrous Na₂SO₄, filtered and concentrated under reduced pressure. The residue was purified by Combi-flash (using gradient elution of 0-40% of EtOAc in n-heptane) to afford desired compound I-61 (3.0 g) as off-white solid. ¹H NMR (400 MHz, DMSO-d₆): δ 10.17 (d, J=2.0 Hz, 1H); 7.93-7.89 (m, 1H); 7.69-7.67 (m, 1H); 1.39 (s, 18H).

### Intermediate-62: Preparation of tert-butyl (tert-butoxycarbonyl)(2,4-difluoro-3-formylphenyl)carbamate (I-62)

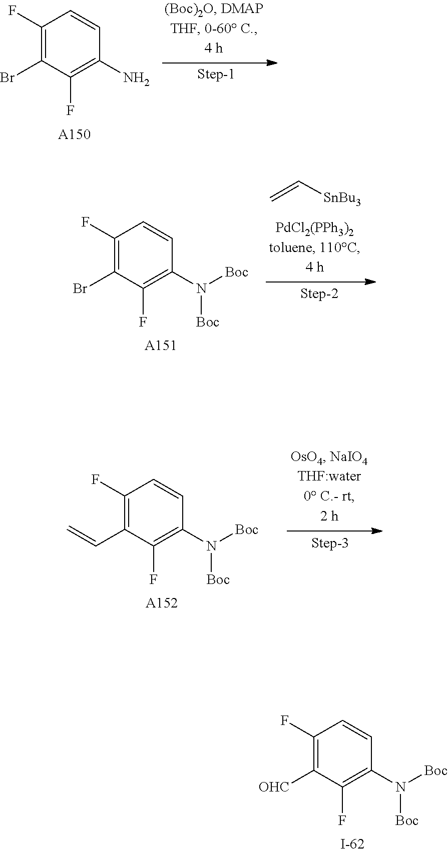

Step-1: Synthesis of tert-butyl (3-bromo-2,4-difluorophenyl)(tert-butoxycarbonyl)carbamate (A151): Compound A151 (16.8 g) was synthesized by following procedure as described for the synthesis of I-61 (step-2) using 3-bromo-2,4-difluoroaniline (A150) (10.0 g, 48.1 mmol) and di-tert-butyl dicarbonate (22.1 mL, 96.2 mmol) as starting materials. ¹H NMR (400 MHz, DMSO-cd₆): δ 7.52-7.59 (m, 1H); 7.34-7.42 (m, 1H); 1.40 (s, 9H); 1.48 (s, 9H).

Step-2: Synthesis of tert-butyl (tert-butoxycarbonyl)(2,4-difluoro-3-vinylphenyl)carbamate (A152): Compound A152 (4.5 g) was synthesized by following procedure as described for the synthesis of I-61 (step-3) using tert-butyl (3-bromo-2,4-difluorophenyl)(tert-butoxycarbonyl)carbamate (A151) (10.0 g, 24.5 mmol) and tributyl(vinyl)stannane (7.16 mL, 24.5 mmol) as starting materials. ¹H NMR (400 MHz, DMSO-d₅): δ 7.34-7.43 (m, 1H); 7.15-7.25 (m, 1H); 6.70 (dd, J=18.0, 11.6 Hz, 1H); 6.0 (d, J=18.0 Hz, 1H); 5.69-5.76 (m, 1H); 1.39 (s, 18H).

Step-3: Synthesis of tert-butyl (tert-butoxycarbonyl)(2,4-difluoro-3-formylphenyl)carbamate (1-62): Intermediate 1-62 (4.5 g) was synthesized by following procedure as described for the synthesis of I-61 (step-4) using tert-butyl (tert-butoxycarbonyl)(2,4-difluoro-3-vinylphenyl)carbamate (A152) (4.50 g, 12.7 mmol) as starting material. ¹H NMR (400 MHz, DMSO-d₆): δ 10.21 (s, 1H); 7.85 (dt, J=5.6, 2.8 Hz, 1H); 7.33 (t, J=6.0 Hz, 1H); 1.39 (s, 18H).

### Intermediate-64: Preparation of 1-[3,5-bis(benzyloxy)-2-pyridyl]cyclopropylamine (I-64)

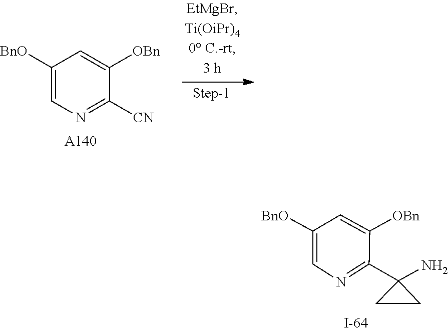

Step-1: Synthesis of 1-[3,5-bis(benzyloxy)-2-pyridyl]cyclopropylamine (1-64): To a stirred solution of 3,5-bis(benzyloxy)-2-pyridinecarbonitrile (A140) (10 g, 31.6 mmol) in anhydrous THE (100 mL) under Argon atmosphere was added Ti(OⁱPr)₄ (10.8 g, 37.9 mmol) at ambient temperature. After stirring the reaction mixture for 15 min, EtMgBr (1.0 M, in THF) (79 mL, 79 mmol) was added slowly via syringe at room temperature, (during addition of EtMgBr, reaction mass becomes black). The reaction mixture was stirred for 1 h, before addition of BF₃.Et₂O (45%) (24.9 g, 79 mmol) at 0° C. The reaction mixture was then allowed to warm to room temperature over 1 h. It was then quenched with addition of water (200 mL) and the reaction mass was filtered through Celite. The filtrate was then made alkaline using 10% NaOH solution (pH=9) and extraction was carried out using DCM (3×150 mL). The combined organic extracts were washed with brine solution (100 mL), dried over anhydrous sodium sulfate, filtered and concentrated under reduced pressure to afford crude product 1-[3,5-bis(benzyloxy)-2-pyridyl]cyclopropylamine (I-64) (11 g), which was used in the next step without further purification. LCMS (ES) m/z: 347.2 [M+H]⁺.

### Example 1: Preparation of 3-(2-fluoro-3-((N-methylsulfamoyl)amino)benzyl)-2-oxo-3,4-dihydro-2H-benzo[e][1,3]oxazin-7-yl dimethylcarbamate (E-01; Compound 156)

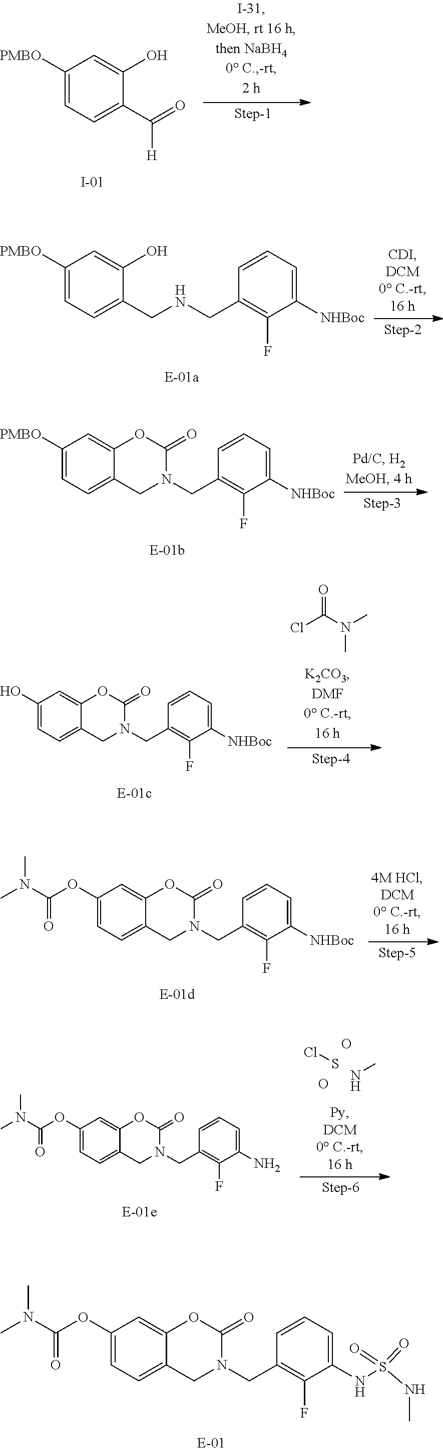

Step-1: Synthesis of tert-butyl (2-fluoro-3-(((2-hydroxy-4-((4-methoxybenzyl)oxy) benzyl)amino)methyl)phenyl)carbamate (E-01a): To a stirred solution of 2-hydroxy-4-[(4-methoxyphenyl)methoxy]benzaldehyde (I-01) (1 g, 3.87 mmol) in MeOH (30 mL) was added tert-butyl (3-(aminomethyl)-2-fluorophenyl)carbamate (1-31) (930 mg, 3.87 mmol) and the reaction mixture was stirred at room temperature for 16 h. To this was then added NaBH₄ (293 mg, 7.74 mmol) at 0° C. and the reaction mixture was allowed to warm to room temperature over 2 h. After complete consumption of starting material, it was quenched with water (30 mL) and MeOH was then removed under reduced pressure. Extraction was carried out using EtOAc (2×50 mL); the combined organic extracts were washed with brine solution (2×50 mL), dried over anhydrous Na₂SO₄, filtered and concentrated under reduced pressure. The residue was purified by Combi-Flash (using gradient elution of 0-40% of EtOAc in heptane) to afford the desired compound E-01a (1.1 g) as an off-white solid. LCMS m/z found 483.3 [M+H]⁺.

Step-2: Synthesis of tert-butyl (2-fluoro-3-((7-((4-methoxybenzyl)oxy)-2-oxo-2H-benzo[e][1,3]oxazin-3(4H)-yl)methyl)phenyl)carbamate (E-01b): To a stirred solution of tert-butyl N-(2-fluoro-3-{[({2-hydroxy-4-[(4-methoxyphenyl) methoxy]phenyl}methyl)amino]methyl}phenyl) carbamate (E-01a) (550 mg, 1.14 mmol) in DCM (12 mL) was added CDI (370 mg, 2.28 mmol) at room temperature and the reaction mixture was stirred at room temperature for 16 h. Volatiles were then evaporated under reduced pressure and the residue was purified by Combi-Flash (using gradient elution of 0-5% of MeOH in DCM) to afford the desired compound E-01b (450 mg) as a pale yellow thick oil. LCMS (ES) m/z: 453.1 [(M-^{t}Bu)+H]⁺.

Step-3: Synthesis of tert-butyl (2-fluoro-3-((7-hydroxy-2-oxo-2H-benzo[e][1,3]oxazin-3(4H)-yl)methyl)phenyl)carbamate (E-01c): To a stirred solution of tert-butyl N-[2-fluoro-3-({7-[(4-methoxyphenyl)methoxy]-2-oxo-3,4-dihydro-2H-1,3-benzoxazin-3-yl}methyl)phenyl]carbamate (E-01b) (450 mg, 885 μmol) in MeOH (10 mL) was added 10% Pd/C (150 mg, 1.41 mmol) and the reaction mixture was stirred under hydrogen atmosphere. The reaction progress was monitored by TLC/LCMS. After complete consumption of starting material (2 h), the catalyst was filtered off through a celite pad and washed with MeOH (5 mL×2). MeOH was then evaporated under reduced pressure and the residue was purified by Combi-Flash (using gradient elution of 0-70% of EtOAc in heptane) to afford the desired compound E-01c (310 mg) as an off-white solid. LCMS (ES) m/z: 387.1 [M−H]⁺.

Step-4: Synthesis of 3-(3-((tert-butoxycarbonyl)amino)-2-fluorobenzyl)-2-oxo-3,4-dihydro-2H-benzo[e][1,3]oxazin-7-yl dimethylcarbamate (E-Old): To a stirred solution of tert-butyl N-{2-fluoro-3-[(7-hydroxy-2-oxo-3,4-dihydro-2H-1,3-benzoxazin-3-yl)methyl]phenyl}carbamate (E-01c) (1 g, 2.57 mmol) in acetonitrile (20 mL, 383 mmol) was added K₂CO₃ (712 mg, 5.15 mmol) and N,N-dimethylcarbamoyl chloride (356 μL, 3.86 mmol) at 0° C. The reaction mixture was then stirred at 80° C. The reaction progress was monitored by TLC/LCMS analysis. After completion of the reaction (4 h), it was cooled to room temperature and water (30 mL) was added to it. Extraction was carried out using EtOAc (2×30 mL); the combined organic extracts were washed with brine solution (30 mL), dried over anhydrous Na₂SO₄, filtered and concentrate under reduced pressure. The residue was purified by Combi-Flash (using gradient elution of 0-80% of EtOAc in heptane) to afford desired compound E-Old (1 g) as an off-white solid. LCMS (ES) m/z: 404.1 1 (M-rBu)+HJ+.

Step-5: Synthesis of 3-(3-amino-2-fluorobenzyl)-2-oxo-3,4-dihydro-2H-benzo[e][1,3]oxazin-7-yl dimethylcarbamate (E-Ole): To a stirred solution of 3-[(3-{I[(tert-butoxy)carbonyl]amino}-2-fluorophenyl)methyl]-2-oxo-3,4-dihydro-2H-1,3-benzoxazin-7-yl N,N-dimethylcarbamate (E-Old) (1 g, 2.18 mmol) in 1,4-dioxane (5 mL) was added a 4 M solution of HCl in 1,4-dioxane (1 mL) at 0° C. The reaction mixture was stirred at room temperature, while monitoring reaction progress by TLC. After complete consumption of starting material (2 h), volatiles were removed under reduced pressure and saturated NaHCO₃ solution (10 mL) was added to the residue. Extraction was carried out using EtOAc (20 mL×3); the combined organic extracts were washed with brine (30 mL), dried over anhydrous Na₂SO₄, filtered and concentrated under reduced pressure. The residue was triturated with diethyl ether (10 mL), filtered and dried to provide desired compound E-Ole (750 mg) as an off-white solid. LCMS (ES) m/z: 360.1 [M+H]⁺.

Step-6: Synthesis of 3-(2-fluoro-3-((N-methylsulfamoyl)amino)benzyl)-2-oxo-3,4-dihydro-2H-benzo[e][1,3]oxazin-7-yl dimethylcarbamate (E-01): To a stirred solution of 3-[(3-amino-2-fluorophenyl)methyl]-2-oxo-3,4-dihydro-2H-1,3-benzoxazin-7-yl N,N-dimethylcarbamate (E-01e) (172 mg, 479 μmol) in DCM (5 mL) was added pyridine (193 μL, 2.39 mmol) and N-methylsulfamoyl chloride (82.6 μL, 957 μmol) at 0° C. The reaction mixture was then allowed to warm to room temperature. The reaction progress was monitored by TLC/LCMS. After complete consumption of starting material (6 h), water (15 mL) was added to it and extraction was carried out using EtOAc (3×20 mL). The combined organic extracts were washed with brine (30 mL), dried with anhydrous Na₂SO₄, filtered and concentrated under the vacuum. The residue was purified by Combi-Flash (using gradient elution of 0-7% of MeOH in DCM) followed by reverse phase preparative HPLC [using XBridge C18 (19 mm×250 mm, 5 μm) particle size column, 0.1% ammonia in water and acetonitrile as mobile phase with 19 mL/min flow rate] to afford desired compound E-01 (22 mg) as an off-white solid. LCMS (ES) m/z: 453.2 [M+H]⁺. ¹H NMR (400 MHz, DMSO-d₆): δ 9.42 (s, 1H); 7.41-7.37 (m, 1H); 7.26-7.12 (m, 4H); 6.92-6.88 (m, 2H); 4.67 (s, 2H); 4.48 (s, 2H); 3.03 (s, 3H); 2.91 (s, 3H); 2.51 (d, J=4.4 Hz, 3H).

### Example 2: Preparation of 8-fluoro-3-(2-fluoro-3-((N-methylsulfamoyl)amino)benzyl)-2-oxo-3,4-dihydro-2H-benzo[e][1,3]oxazin-7-yl dimethylcarbamate (E-02; Compound 131)

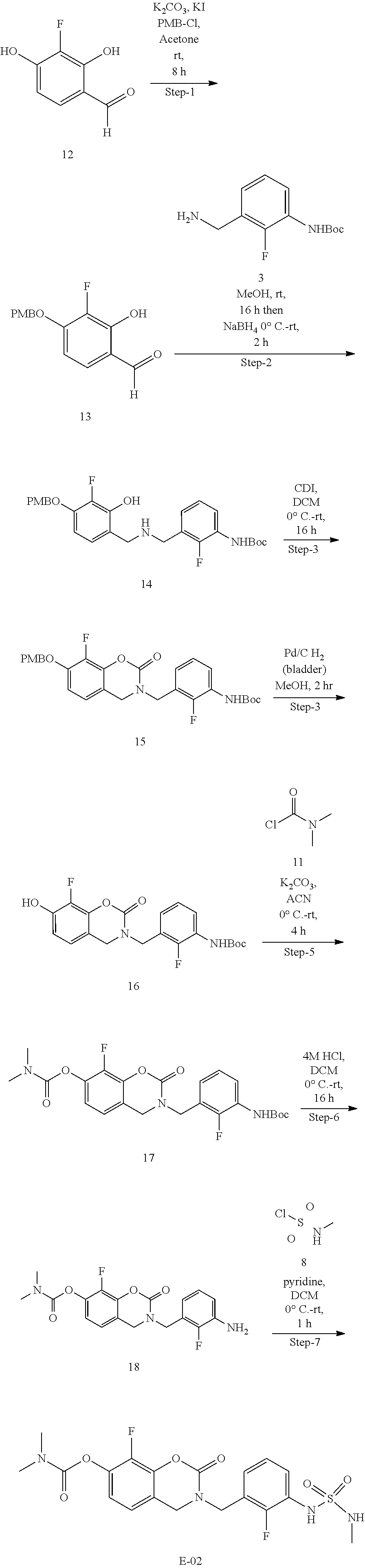

Step-1: Synthesis of 3-fluoro-2-hydroxy-4-[(4-methoxyphenyl)methoxy]benzaldehyde (13): Compound 13 (0.5 g) was synthesized by following procedure as described for the synthesis of E-01 (step-1) using 2-fluoro-4,6-dihydroxybenzaldehyde (12) (0.7 g, 4.48 mmol) as the starting material. LCMS (ES) m/z: 275.0 [M−H]+.

Step-2: Synthesis of N-(2-fluoro-3-{[({3-fluoro-2-hydroxy-4-[(4-methoxyphenyl)methoxy]phenyl}methyl)amino]methyl}phenyl)carbamate (14): Compound 14 (0.48 g) was synthesized by following procedure as described for the synthesis of E-01 (step-2) using 2-fluoro-6-hydroxy-4-[(4-methoxyphenyl)methoxy]benzaldehyde (13) (0.3 g, 1.09 mmol) and tert-butyl N-[3-(aminomethyl)-2-fluorophenyl]carbamate (3) (0.26 g, 1.09 mmol) as the starting materials. LCMS (ES) m/z: 501.2 [M+H]⁺.

Step-3: Synthesis of tert-butyl N-[2-fluoro-3-({8-fluoro-7-[(4-methoxyphenyl)methoxy]-2-oxo-3,4-dihydro-2H-1,3-benzoxazin-3-yl}methyl)phenyl]carbamate (15): Compound 15 (0.26 g) was synthesized by following procedure as described for the synthesis of E-01 (step-3) using N-(2-fluoro-3-{[(3-fluoro-2-hydroxy-4-[(4-methoxyphenyl)methoxy]phenyl}methyl)amino]methyl}phenyl)carbamate (14) (0.25 g, 499 μmol) as the starting material. LCMS (ES) m/z: 471.1 [(M-^{t}Bu)+H]⁺.

Step-4: Synthesis of tert-butyl N-{2-fluoro-3-[(8-fluoro-7-hydroxy-2-oxo-3,4-dihydro-2H-1,3-benzoxazin-3-yl) methyl]phenyl}carbamate (16): To a stirred solution of tert-butyl N-[2-fluoro-3-({8-fluoro-7-[(4-methoxyphenyl)methoxy]-2-oxo-3,4-dihydro-2H-1,3-benzoxazin-3-yl}methyl)phenyl]carbamate (15) (0.3 g, 570 μmol) in MeOH (10 mL) was added 10% palladium on carbon (0.12 g, 1.14 mmol) and the reaction mixture was allowed to stir at room temperature under hydrogen atmosphere. The reaction progress was monitored by TLC/LCMS analysis. After completion of the reaction (2 h), the catalyst was filtered off through a Celite pad and washed with MeOH (5 mL×2). The filtrate was evaporated under reduced pressure and the residue was purified by Combi-Flash (using gradient elution of 0-5% of MeOH in DCM) to afford desired compound 16 (0.23 g) as an off-white solid. LCMS (ES) m/z: 351.1 [(M-^{t}Bu)+H]⁺.

Step-5: Synthesis of 3-[(3-{[(tert-butoxy) carbonyl]amino}-2-fluorophenyl) methyl]-8-fluoro-2-oxo-3,4-dihydro-2H-1,3-benzoxazin-7-yl N,N-dimethyl carbamate (17): Compound 17 (0.22 g) was synthesized by following procedure as described for the synthesis of E-01 (step-8) using tert-butyl N-{2-fluoro-3-[(8-fluoro-7-hydroxy-2-oxo-3,4-dihydro-2H-1,3-benzoxazin-3-yl)methyl]phenyl}carbamate (16) (0.23 g, 492 μmol) as the starting material. LCMS (ES) m/z: 422.1 [(M-^{t}Bu)+H]⁺.

Step-6: Synthesis of 3-[(3-amino-2-fluorophenyl) methyl]-8-fluoro-2-oxo-3,4-dihydro-2H-1,3-benzoxazin-7-yl N,N-dimethyl carbamate (18): To a stirred solution of 3-[(3-{[(tert-butoxy)carbonyl]amino}-2-fluorophenyl)methyl]-8-fluoro-2-oxo-3,4-dihydro-2H-1,3-benzoxazin-7-yl N,N-dimethyl carbamate (17) (0.22 g, 461 mol) in 1,4-dioxane (1 mL) was added 4 M solution of HCl in 1,4-dioxane (64 μL, 1.84 mmol) at 0° C. The reaction mixture was stirred at room temperature for 16 h. After completion of the reaction (as indicated by TLC), volatiles were concentrated under reduced pressure and saturated sodium bicarbonate solution (20 mL) was added to the residue. Extraction was carried out using EtOAc (30 mL×2); the combined organic extracts were washed with brine (30 mL), dried over anhydrous Na₂SO₄, filtered and concentrated under reduced pressure. The residue was triturated with diethyl ether (3×10 mL) and dried to obtain the desired compound 18 (0.130 g) as an off-white solid. LCMS (ES) m/z: 378.1 [M+H]⁺.

Step-7: Synthesis of 3-(2-fluoro-3-((N-methylsulfamoyl)amino)benzyl)-2-oxo-3,4-dihydro-2H-benzo[e][1,3]oxazin-7-yl dimethylcarbamate (E-02): E-02 (0.05 g) was synthesized by following procedure as described for the synthesis of E-01 (step-6) using 3-(3-amino-2-fluorophenyl)methyl]-8-fluoro-2-oxo-3,4-dihydro-2H-1,3-benzoxazin-7-yl N,N-dimethyl carbamate (18) (0.13 g, 345 μmol) as the starting material. Reverse phase preparative HPLC purification method: XSelect C18 (20 mm×250 mm, 5 μm particle size) column, 0.1% ammonia in water and acetonitrile as mobile phase with 18 mL/min flow rate. LCMS (ES) m/z: 471.3 [M+H]⁺. ¹H NMR (400 MHz, DMSO-d₆): δ 9.43 (s, 1H); 7.42-7.37 (m, 1H); 7.23-7.13 (m, 3H); 7.07-7.02 (m, 2H); 4.68 (s, 2H); 4.53 (s, 2H); 3.06 (s, 3H); 2.92 (s, 3H); 2.52 (d, J=4.8 Hz, 3H).

### Example 3: Preparation of 5-fluoro-3-(2-fluoro-3-((N-methylsulfamoyl)amino)benzyl)-2-oxo-3,4-dihydro-2H-benzo[e][1,3]oxazin-7-yl dimethylcarbamate (E-03; Compound 118)

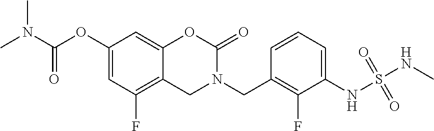

E-03 was synthesized by following synthesis sequence and experimental procedures as described for the synthesis of E-01, using key intermediates I-06 and I-31. LCMS (ES) m/z: 471.1 [M+H]⁺. ¹H NMR (400 MHz, DMSO-d₆): δ 9.41 (s, 1H); 7.42-7.38 (m, 1H); 7.22-7.12 (m, 3H); 6.94 (dd, J₁=2.0 Hz, J_{z}=10.0 Hz, 1H); 6.80 (s, 1H); 4.69 (s, 2H); 4.49 (s, 2H); 3.02 (s, 3H); 2.90 (s, 3H); 2.51 (d, J=4.4 Hz, 3H).

### Example 4: Preparation of 3-(2-fluoro-3-((N-methylsulfamoyl)amino)benzyl)-6-methyl-2-oxo-3,4-dihydro-2H-benzo[e][1,3]oxazin-7-yl dimethylcarbamate (E-04; Compound 112)

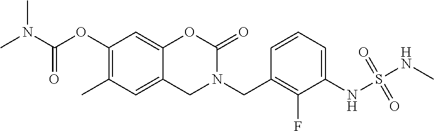

E-04 was synthesized by following synthesis sequence and procedures as described for the synthesis of E-01, using key intermediates I-08 and I-31. LCMS (ES) m/z: 467.2 [M+H]⁺. ¹H NMR (400 MHz, DMSO-d₆): δ 9.39 (s, 1H); 7.40-7.36 (m, 1H); 7.22-7.18 (m, 1H); 7.16-7.10 (m, 3H); 6.83 (s, 1H); 4.66 (s, 2H); 4.44 (s, 2H); 3.05 (s, 3H); 2.91 (s, 3H); 2.51 (d, J=4.4 Hz, 3H); 2.06 (s, 3H).

### Example 5: Preparation of 3-(3-(cyclopropanesulfonamido)-2-fluorobenzyl)-2-oxo-3,4-dihydro-2H-benzo[e][1,3]oxazin-7-yl dimethylcarbamate (E-05; Compound 125)

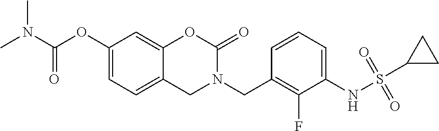 [SMILES: CN(C)C(=O)Oc1ccc2c(c1)OC(=O)N(Cc1cccc(NS(=O)(=O)C3CC3)c1F)C2]

E-05 was synthesized by following synthesis sequence and procedures as described for the synthesis of E-01, using intermediate E-Ole and cyclopropylsulfonylchloride as the starting materials. LCMS (ES) m/z: 464.1 [M+H]⁺. ¹H NMR (400 MHz, DMSO-d₆): δ 9.66 (s, 1H); 7.39-7.35 (m, 1H); 7.23-7.13 (m, 3H); 6.19-6.87 (m, 2H); 4.68 (s, 2H); 4.47 (s, 2H); 3.02 (s, 3H); 2.90 (s, 3H); 2.67-2.59 (m, 1H); 0.92-0.85 (m, 4H).

### Example 6: Preparation of 3-(3-(cyclopropanesulfonamido)-2-fluorobenzyl)-5-fluoro-2-oxo-3,4-dihydro-2H-benzo[e][1,3]oxazin-7-yl dimethylcarbamate (E-06; Compound 102)

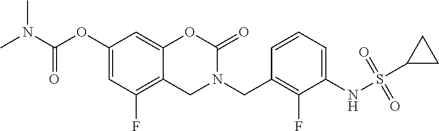 [SMILES: CN(C)C(=O)Oc1cc(F)c2c(c1)OC(=O)N(Cc1cccc(NS(=O)(=O)C3CC3)c1F)C2]

E-06 was synthesized by following synthesis sequence and experimental procedures as described for the synthesis of E-01, using key intermediates I-06 and I-31. (Note: cyclopropylsulfonyl chloride was used in final step). LCMS (ES) m/z: 482.3 [M+H]⁺.

¹H NMR (400 MHz, DMSO-d₆): δ 9.59 (s, 1H); 7.40-7.36 (m, 1H); 7.31-7.24 (m, 1H); 7.19-7.15 (m, 1H); 7.00-6.92 (m, 1H); 6.81-6.80 (m, 1H); 4.71 (s, 2H); 4.49 (s, 2H); 3.16 (s, 3H); 2.90 (s, 3H); 2.68-2.60 (m, 1H); 0.94-0.84 (m, 4H).

### Example 7: Preparation of 3-(2-fluoro-3-((3-fluoropyrrolidine)-1-sulfonamido)benzyl)-2-oxo-3,4-dihydro-2H-benzo[e][1,3]oxazin-7-yl dimethylcarbamate (E-07; Compound 113)

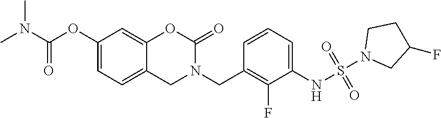 [SMILES: CN(C)C(=O)Oc1ccc2c(c1)OC(=O)N(Cc1cccc(NS(=O)(=O)N3CCC(F)C3)c1F)C2]

E-07 was synthesized by following synthesis sequence and procedures as described for the synthesis of E-01, using E-Ole and 3-fluoropyrrolidine-1-sulfonyl chloride as the starting materials. LCMS (ES) m/z: 511.2 [M+H]⁺. ¹H NMR (400 MHz, DMSO-d₆): b 9.76 (s, 1H); 7.41 (td, J₁=1.6 Hz, J₂=8.0 Hz, 1H); 7.23-7.13 (m, 3H); 6.92-6.88 (m, 2H); 5.32-5.16 (m, 1H); 4.66 (s, 2H); 4.45 (s, 2H); 3.42 (d, J=2.0 Hz, 1H); 3.38-3.35 (m, 2H); 3.28-3.23 (m, 1H); 3.02 (s, 3H); 2.90 (s, 3H); 2.06-1.90 (m, 2H).

### Example 8: E-08: Preparation of 3-(2-fluoro-3-(morpholine-4-sulfonamido)benzyl)-2-oxo-3,4-dihydro-2H-benzo[e][1,3]oxazin-7-yl dimethylcarbamate (E-08; Compound 110)

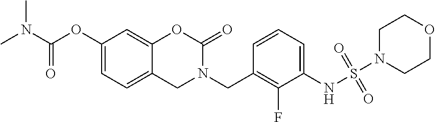 [SMILES: CN(C)C(=O)Oc1ccc2c(c1)OC(=O)N(Cc1cccc(NS(=O)(=O)N3CCOCC3)c1F)C2]

E-08 was synthesized by following synthesis sequence and procedures as described for the synthesis of E-01, using intermediate E-Ole and morpholine-4-sulfonyl chloride as the starting materials. LCMS (ES) m/z: 509.2 [M+H]⁺. ¹H NMR (400 MHz, DMSO-d₆): δ 9.82 (s, 1H); 7.41 (td, J₁=1.6 Hz, J₂=7.6 Hz, 1H); 7.23-7.15 (m, 3H); 6.91-6.88 (m, 2H); 4.68 (s, 2H); 4.47 (s, 2H); 3.54-3.52 (m, 4H); 3.05-3.02 (m, 4H); 3.02 (s, 3H); 2.90 (s, 3H).

### Example 9: Preparation of 3-(2-fluoro-3-(2-oxa-6-azaspiro[3.3]heptane-6-sulfonamido)benzyl)-2-oxo-3,4-dihydro-2H-benzo[e][1,3]oxazin-7-yl dimethylcarbamate (E-09; Compound 109)

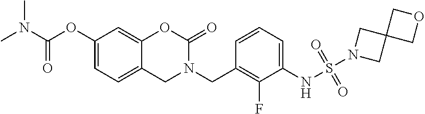 [SMILES: CN(C)C(=O)Oc1ccc2c(c1)OC(=O)N(Cc1cccc(NS(=O)(=O)N3CC4(COC4)C3)c1F)C2]

E-09 was synthesized by following synthesis sequence and procedures as described for the synthesis of E-01, using intermediate E-Ole and 2-oxa-6-azaspiro[3.3]heptane-6-sulfonyl chloride as the starting materials. LCMS (ES) m/z: 521.2 [M+H]⁺. ¹H NMR (400 MHz, DMSO-d₆): δ 9.75 (br s, 1H); 7.40-7.35 (m, 1H); 7.23 (d, J=8.4 Hz, 1H); 7.20-7.12 (m, 2H); 6.92-6.88 (m, 2H); 4.66 (s, 2H); 4.60 (s, 4H); 4.49 (s, 2H); 3.95 (s, 4H); 3.02 (s, 3H); 2.90 (s, 3H).

### Example 10: Preparation of 3-(2-fluoro-3-((3-methoxypyrrolidine)-1-sulfonamido)benzyl)-2-oxo-3,4-dihydro-2H-benzo[e][1,3]oxazin-7-yl dimethylcarbamate (E-10; Compound 100)

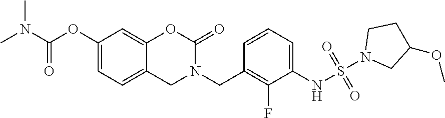

E-10 was synthesized by following synthesis sequence and procedures as described for the synthesis of E-01, using intermediate E-Ole and 3-methoxypyrrolidine-1-sulfonyl chloride as the starting materials. LCMS (ES) m/z: 523.3 [M+H]⁺. ¹H NMR (400 MHz, DMSO-d₆): δ 9.66 (s, 1H); 7.43 (td, J₁=2.0 Hz, J₂=8.0 Hz, 1H); 7.24-7.13 (m, 3H); 6.92-6.88 (m, 2H); 4.67 (s, 2H); 4.47 (s, 2H); 3.86-3.84 (m, 1H); 3.33-3.14 (m, 4H); 3.12 (s, 3H); 3.03 (s, 3H); 2.91 (s, 3H); 1.84-1.78 (m, 2H).

### Example 11: Preparation of 3-(2-fluoro-3-((N-methylsulfamoyl)amino)benzyl)-2-oxo-3,4-dihydro-2H-benzo[e][1,3]oxazin-7-yl cyclopropanecarboxylate (E-11; Compound 147)

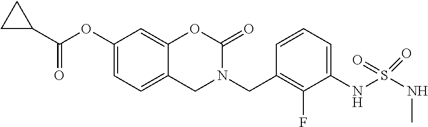 [SMILES: CNS(=O)(=O)Nc1cccc(CN2Cc3ccc(OC(=O)C4CC4)cc3OC2=O)c1F]

E-11 was synthesized by following synthesis sequence and procedures as described for the synthesis of E-01, using key intermediate E-01c and cyclopropylcarbonyl chloride as the starting materials. LCMS (ES) m/z: 448.2 [M−H]⁺. ¹H NMR (400 MHz, DMSO-d₆): δ 9.43 (s, 1H); 7.41-7.37 (m, 1H); 7.27 (d, J=8.8 Hz, 1H); 7.22 (d, J=4.8 Hz, 1H); 7.15 (t, J=3.2 Hz, 2H); 6.93 (t, J=6.8 Hz, 2H); 4.67 (s, 2H); 4.48 (s, 2H); 2.52 (d, J=4.4 Hz, 3H); 1.92-1.86 (m, 1H); 1.09-1.01 (m, 4H).

### Example 12: Preparation of 3-({2-fluoro-3-[(methylsulfamoyl)amino]phenyl}methyl)-6-methyl-7-(pyrimidin-2-yloxy)-3,4-dihydro-2H-1,3-benzoxazin-2-one (E-12; Compound 111)

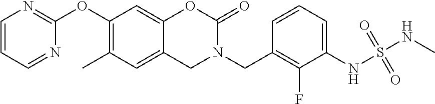 [SMILES: CNS(=O)(=O)Nc1cccc(CN2Cc3cc(C)c(Oc4ncccn4)cc3OC2=O)c1F]

E-12 was synthesized by following similar synthesis sequence and experimental procedures as described for the synthesis of E-01, using key intermediates I-08 and I-31. LCMS (ES) m/z: 474.3 [M+H]⁺. ¹H NMR (400 MHz, DMSO-d₆): δ 9.43 (s, 1H); 8.64 (d, J=4.8 Hz 2H); 7.41-7.37 (m, 1H); 7.27 (t, J=4.8 Hz, 1H); 7.24-7.22 (m, 1H); 7.18-7.13 (m, 3H); 6.93 (s, 1H); 4.87 (s, 2H); 4.47 (s, 2H); 2.52 (d, J=4.4 Hz, 3H); 1.98 (s, 3H).

### Example 13: Preparation of 5-fluoro-3-({2-fluoro-3-(methylsulfamoyl)amino]phenyl}methyl)-7-(pyrimidin-2-yloxy)-3,4-dihydro-2H-1,3-benzoxazin-2-one (E-13; Compound 117)

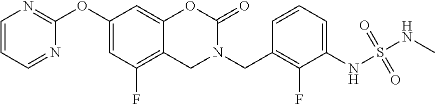 [SMILES: CNS(=O)(=O)Nc1cccc(CN2Cc3c(F)cc(Oc4ncccn4)cc3OC2=O)c1F]

E-13 was synthesized by following similar synthesis sequence and experimental procedures as described for the synthesis of E-01, using key intermediates I-06 and I-31. LCMS (ES) m/z: 478.3 [M+H]⁺. ¹H NMR (400 MHz, DMSO-d₆): δ 9.39 (s, 1H); 8.67 (d, j=4.8 Hz, 2H); 7.42-7.38 (m, 1H); 7.33-7.30 (m, 1H); 7.22-7.13 (m, 3H); 7.08-7.05 (m, 1H); 6.92 (s, 1H); 4.70 (s, 2H); 4.53 (s, 2H); 2.52 (d, J=4.8 Hz, 3H).

### Example 14: Preparation of 3-({2-fluoro-3-[(methylsulfamoyl)amino]phenyl}methyl)-7-(1,3-oxazol-2-yloxy)-3,4-dihydro-2H-1,3-benzoxazin-2-one (E-14; Compound 80)

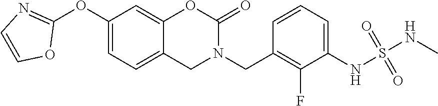 [SMILES: CNS(=O)(=O)Nc1cccc(CN2Cc3ccc(Oc4ncco4)cc3OC2=O)c1F]

E-14 was synthesized by following similar synthesis sequence and experimental procedures as described for the synthesis of E-01, using intermediate E-01c and 2-(methylsulfonyl)oxazole as the starting materials. LCMS (ES) m/z: 449.3 [M+H]⁺. ¹H NMR (400 MHz, DMSO-d₆): δ 9.41 (s, 1H); 7.83 (d, J=0.8 Hz 1H); 7.41-7.39 (m, 1H); 7.38-7.31 (m, 1H); 7.21-7.16 (m, 5H); 7.12 (s, 1H); 4.67 (s, 2H); 4.50 (s, 2H); 2.52 (d, J=4.8 Hz, 3H).

### Example 15: Preparation of 3-({2-fluoro-3-[(methylsulfamoyl)amino]phenyl}methyl)-7-(pyridin-2-yloxy)-3,4-dihydro-2H-1,3-benzoxazin-2-one (E-15; Compound 65)

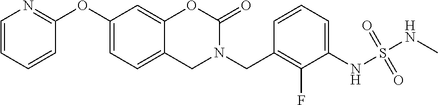 [SMILES: CNS(=O)(=O)Nc1cccc(CN2Cc3ccc(Oc4ccccn4)cc3OC2=O)c1F]

E-15 was synthesized by following similar synthesis sequence and experimental procedures as described for the synthesis of E-01, using key intermediate E-01c and 2-bromopyridine as the starting materials. LCMS (ES) m/z: 459.3 [M+H]⁺. ¹H NMR (400 MHz, DMSO-d₆): δ 9.43 (s, 1H); 8.15-8.14 (m, 1H); 7.89-7.84 (m, 1H); 7.41-7.31 (m, 1H); 7.27-7.25 (m, 1H); 7.20 (s, 1H); 7.16-7.13 (m, 3H); 7.07-7.05 (m, 1H); 6.91-6.89 (m, 2H); 4.67 (s, 2H); 4.49 (s, 2H); 2.52 (d, J=4.8 Hz, 3H).

### Example 16: Preparation of N-(2-fluoro-3-((2-oxo-7-(pyridin-2-yloxy)-2H-benzo[e][1,3]oxazin-3(4H)-yl)methyl)phenyl)cyclopropanesulfonamide (E-16; Compound 62)

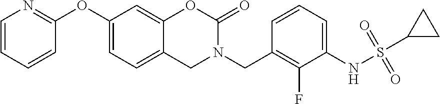 [SMILES: O=C1Oc2cc(Oc3ccccn3)ccc2CN1Cc1cccc(NS(=O)(=O)C2CC2)c1F]

E-16 was synthesized by following synthesis sequence and procedures as described for the synthesis of E-01, using key intermediate E-01c and 2-bromopyridine as the starting materials. (note: cyclopropylsulfonyl chloride was used in last step). LCMS (ES) m/z: 470.3 [M+H]⁺. ¹H NMR (400 MHz, DMSO-d₆): δ 9.64 (s, 1H); 8.16-8.14 (m, 1H); 7.89-7.85 (m, 1H); 7.41-7.37 (m, 1H); 7.27 (d, J=8.0 Hz, 2H); 7.20-7.14 (m, 2H); 7.06 (d, J=8 Hz, 1H); 6.92-6.89 (m, 2H); 4.70 (s, 2H); 4.50 (s, 2H); 2.68-2.62 (m, 1H); 0.95-0.85 (m, 4H).

### Example 17: Preparation of 3-({2-fluoro-3-[(methylsulfamoyl)amino]phenyl}methyl)-7-(pyrimidin-2-yloxy)-3,4-dihydro-2H-1,3-benzoxazin-2-one (E-17; Compound 157)

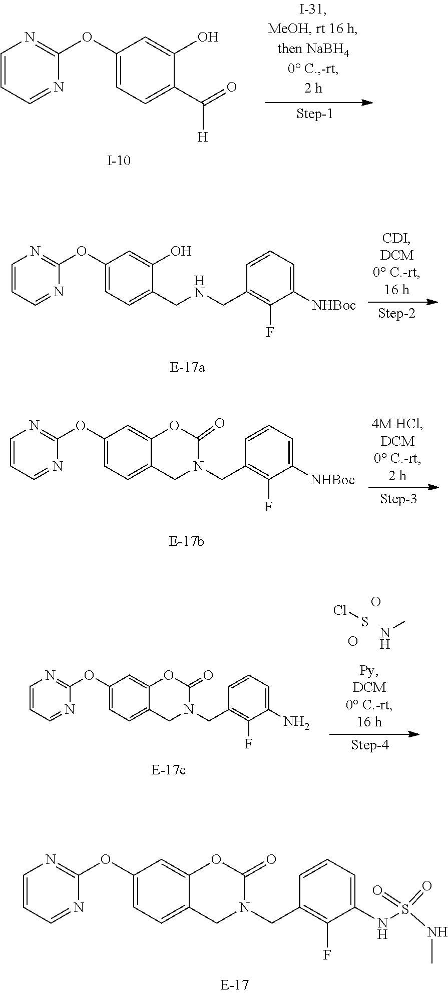

Step-1: Synthesis of tert-butyl (2-fluoro-3-(((2-hydroxy-4-(pyrimidin-2-yloxy)benzyl)amino)methyl)phenyl)carbamate (E-17a): Compound E-17a (0.3 g) was synthesized by following procedure as described for the synthesis of E-01 (step-1) using 2-hydroxy-4-(pyrimidin-2-yloxy)benzaldehyde (I-10) (0.4 g, 1.85 mmol) and I-31 (0.533 g, 2.22 mmol) as the starting materials. LCMS (ES) m/z: 441.2 [M+H]⁺.

Step-2: Synthesis of tert-butyl (2-fluoro-3-((2-oxo-7-(pyrimidin-2-yloxy)-2H-benzo[e][1,3]oxazin-3(4H)-yl)methyl)phenyl)carbamate (E-17b): Compound E-17b (0.150 g) was synthesized by following procedure as described for the synthesis of E-01 (step-2) using tert-butyl (2-fluoro-3-(((2-hydroxy-4-(pyrimidin-2-yloxy)benzyl)amino)methyl)phenyl)carbamate (E-17a) (0.250 g) as the starting material. LCMS (ES) m/z: 467.3 [M+H]⁺.

Step-3: Synthesis of 3-(3-amino-2-fluorobenzyl)-7-(pyrimidin-2-yloxy)-3,4-dihydro-2H-benzo[e][1,3]oxazin-2-one (E-17c): To a stirred solution of tert-butyl N-(2-fluoro-3-{[2-oxo-7-(pyrimidin-2-yloxy)-3,4-dihydro-2H-1,3-benzoxazin-3-yl]methyl}phenyl)carbamate (E-17b) (250 mg, 536 μmol) in 1,4-dioxane (2 mL) was added a 4 M solution of HCl in 1,4-dioxane (2.5 mL) at 0° C. and the reaction mixture was allowed to stir at room temperature. The reaction progress was monitored by TLC/LCMS. After complete consumption of starting material (2 h), volatiles were removed under reduced pressure and saturated NaHCO₃ solution (10 mL) was added to the residue. Extraction was carried out using DCM (3×50 mL); the combined organic extracts were washed with brine solution (2×20 mL), dried over anhydrous Na₂SO₄, filtered and concentrated under reduced pressure. The residue was purified by Combi-Flash (using gradient elution of 0-50% of EtOAc in heptane) to afford the desire compound E-17c (0.1 g) as an off-white solid. LCMS (ES) m/z: 367.2 [M+H]⁺.

Step-4: Synthesis of 3-({2-fluoro-3-[(methylsulfamoyl)amino]phenyl}methyl)-7-(pyrimidin-2-yloxy)-3,4-dihydro-2H-1,3-benzoxazin-2-one (E-17): Example E-17 (0.055 g) was synthesized by following procedure as described for the synthesis of E-01 (step-6) using 3-(3-amino-2-fluorobenzyl)-7-(pyrimidin-2-yloxy)-3,4-dihydro-2H-benzo[e][1,3]oxazin-2-one (E-17c) (0.2 g, 546 μmol) as the starting material. LCMS (ES) m/z: 460.2 [M+H]⁺. ¹H NMR (400 MHz, DMSO-d₆): δ 9.41 (s, 1H); 8.63 (d, J=4.8 Hz, 2H); 7.42-7.10 (m, 6H); 7.00-6.92 (m, 2H); 4.66 (s, 2H); 4.49 (s, 2H); 2.50 (d, J=4.8 Hz, 3H).

### Example 18: Preparation of 8-fluoro-3-{[2-fluoro-3-(methylaminosulfonylamino)phenyl]methyl}-7-(2-pyrimidinyloxy)-3,4-dihydro-2H-1,3-benzoxazin-2-one (E-18; Compound 123)

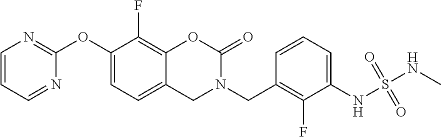 [SMILES: CNS(=O)(=O)Nc1cccc(CN2Cc3ccc(Oc4ncccn4)c(F)c3OC2=O)c1F]

E-18 was synthesized by following similar synthesis sequence and experimental procedures as described for the synthesis of E-17, using key intermediates I-11 and I-31. LCMS (ES) m/z: 478.3 [M+H]⁺. ¹H NMR (400 MHz, DMSO-d₆): δ 9.40 (s, 1H); 8.65 (d, J=4.4 Hz, 2H); 7.39-7.29 (m, 2H); 7.20-7.08 (m, 5H); 4.65 (s, 2H); 4.53 (s, 2H); 2.50 (d, J=4.8 Hz, 3H).

### Example 19: Preparation of 3-{[2-fluoro-3-(methylaminosulfonylamino)phenyl]methyl}-7-(3-pyridyloxy)-3,4-dihydro-2H-1,3-benzoxazin-2-one (E-19; Compound 61)

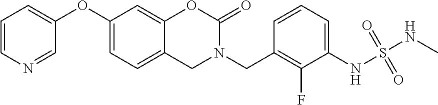 [SMILES: CNS(=O)(=O)Nc1cccc(CN2Cc3ccc(Oc4cccnc4)cc3OC2=O)c1F]

E-19 was synthesized by following similar synthesis sequence and experimental procedures as described for the synthesis of E-17, using key intermediates I-51 and I-31. LCMS (ES) m/z: 459.3 [M+H]⁺. ¹H NMR (400 MHz, DMSO-d₆): δ 9.43 (s, 1H); 8.39 (d, J=2.0 Hz, 2H); 7.48-7.37 (m, 3H); 7.27 (d, J=2 Hz, 1H); 7.21-7.19 (m, 1H); 7.17-7.13 (m, 2H); 6.84-6.80 (m, 2H); 4.66 (s, 2H); 4.47 (s, 2H); 2.50 (d, J=4.8 Hz, 3H).

### Example 20: Preparation of (cyclopropylsulfonyl)(2-fluoro-3-{[2-oxo-7-(3-pyridyloxy)-3,4-dihydro-2H-1,3-benzoxazin-3-yl]methyl}phenyl)amine (E-20; Compound 60)

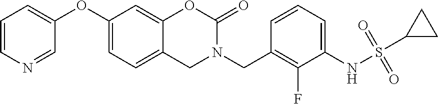 [SMILES: O=C1Oc2cc(Oc3cccnc3)ccc2CN1Cc1cccc(NS(=O)(=O)C2CC2)c1F]

E-20 was synthesized by following similar synthesis sequence and experimental procedures as described for the synthesis of E-17, using key intermediates I-51 and I-31. (note: cyclopropylsulfonyl chloride was used in the last step). LCMS (ES) m/z: 470.3 [M+H]⁺. ¹H NMR (400 MHz, DMSO-d₆): δ 9.62 (s, 1H); 8.38-8.37 (m, 2H); 7.47-7.34 (m, 3H); 7.27-7.22 (m, 2H); 7.18-7.14 (m, 1H); 6.83-6.80 (m, 2H); 4.66 (s, 2H); 4.45 (s, 2H); 2.65-2.61 (m, 1H); 0.90-0.84 (m, 4H).

### Example 21: Preparation of 7-(3,3-dimethylureido)-3-{[2-fluoro-3-(methylaminosulfonylamino) phenyl]methyl}-3,4-dihydro-2H-1,3-benzoxazin-2-one (E-21; Compound 136)

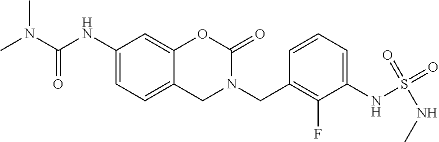 [SMILES: CNS(=O)(=O)Nc1cccc(CN2Cc3ccc(NC(=O)N(C)C)cc3OC2=O)c1F]

E-21 was synthesized by following similar synthesis sequence and experimental procedures as described for the synthesis of E-17, using key intermediates I-17 and I-31. LCMS (ES) m/z: 452.3 [M+H]⁺. ¹H NMR (400 MHz, DMSO-d₆): δ 9.42 (s, 1H); 8.40 (s, 1H); 7.41-7.36 (m, 1H); 7.27-7.21 (m, 3H); 7.16-7.10 (m, 2H); 7.06 (d, J=8.4 Hz, 1H); 4.66 (s, 2H); 4.40 (s, 2H); 2.92 (s, 6H); 2.50 (d, J=4.8 Hz, 3H).

### Example 22: Preparation of 3-{[2-fluoro-3-(methylaminosulfonylamino)phenyl]methyl}-7-(1,3,3-trimethylureido)-3,4-dihydro-2H-1,3-benzoxazin-2-one (E-22; Compound 135)

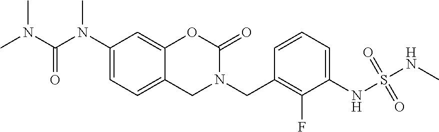

E-22 was synthesized by following similar synthesis sequence and experimental procedures as described for the synthesis of E-17, using key intermediates I-18 and I-31. LCMS (ES) m/z: 466.2 [M+H]⁺. ¹H NMR (400 MHz, DMSO-d₆): δ 9.43 (s, 1H); 7.40-7.36 (m, 1H); 7.23-7.22 (m, 1H); 7.17-7.11 (m, 3H); 6.78 (dd, J₁=2.4 Hz, J₂=8.0 Hz, 1H); 6.73 (d, J=2.4 Hz, 1H); 4.65 (s, 2H); 4.42 (s, 2H); 3.05 (s, 3H); 2.66 (s, 6H); 2.50 (d, J=4.4 Hz, 3H).

### Example 23: Preparation of N,N-dimethyl-3-{[2-fluoro-3-(methylaminosulfonylamino)phenyl]methyl}-2-oxo-3,4-dihydro-2H-1,3-benzoxazine-7-carboxamide (E-23; Compound 121)

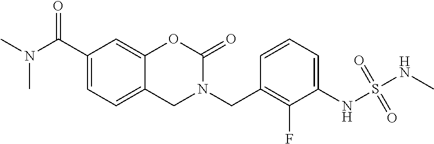

E-23 was synthesized by following similar synthesis sequence and experimental procedures as described for the synthesis of E-17, using key intermediates I-52 and I-31. LCMS (ES) m/z: 437.2 [M+H]⁺. ¹H NMR (400 MHz, DMSO-d₆): δ 9.45 (s, 1H); 7.41-7.37 (m, 1H); 7.29 (d, J=8.0 Hz, 1H); 7.24 (q, J=4.8 Hz, 1H); 7.17-7.12 (m, 3H); 7.08 (d, J=1.2 Hz, 1H); 4.67 (s, 2H); 4.52 (s, 2H); 2.97 (s, 3H); 2.89 (s, 3H); 2.50 (d, J=4.4 Hz, 3H).

### Example 24: Preparation of 3-{[2-fluoro-3-(methylaminosulfonylamino)phenyl]methyl}-7-[(2-oxo-1-pyridyl)methyl]-3,4-dihydro-2H-1,3-benzoxazin-2-one (E-24; Compound 137)

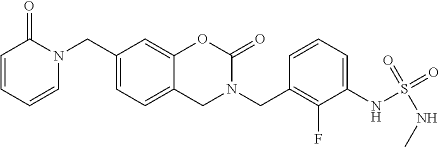 [SMILES: CNS(=O)(=O)Nc1cccc(CN2Cc3ccc(Cn4ccccc4=O)cc3OC2=O)c1F]

E-24 was synthesized by following similar synthesis sequence and experimental procedures as described for the synthesis of E-17, using key intermediates I-14 and I-31. LCMS (ES) m/z: 473.3 [M+H]⁺. ¹H NMR (400 MHz, DMSO-d₆): δ 9.40 (s, 1H); 7.80 (dd, J₁=1.6 Hz, J₂=6.8 Hz, 1H); 7.45-7.35 (m, 2H); 7.21-7.04 (m, 5H); 6.98 (s, 1H); 6.42 (d, J=8.8 Hz, 1H); 6.26 (t, J=6.8 Hz, 1H); 5.07 (s, 2H); 4.66 (s, 2H); 4.46 (s, 2H); 2.50 (d, J=4.4 Hz, 3H).

### Example 25: Preparation of 3-{[2-fluoro-3-(methylaminosulfonylamino)phenyl]methyl}-7-[(2-oxo-1-pyrrolidinyl)methyl]-3,4-dihydro-2H-1,3-benzoxazin-2-one (E-25; Compound 124)

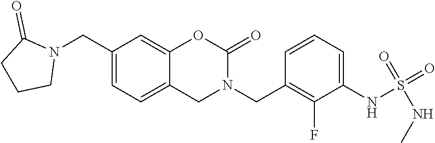 [SMILES: CNS(=O)(=O)Nc1cccc(CN2Cc3ccc(CN4CCCC4=O)cc3OC2=O)c1F]

E-25 was synthesized by following similar synthesis sequence and experimental procedures as described for the synthesis of E-17, using key intermediates I-15 and I-31. LCMS (ES) m/z: 463.3 [M+H]⁺. ¹H NMR (400 MHz, DMSO-d₆): δ 9.39 (s, 1H); 7.40-7.30 (m, 1H); 7.20-7.05 (m, 4H); 6.96 (d, J=8.0, 1H); 6.86 (s, 1H); 4.63 (s, 2H); 4.43 (s, 2H); 4.31 (s, 2H); 3.20 (t, J=6.8, 2H); 2.50 (d, J=4.4 Hz, 3H); 2.25 (t, J=7.6, 2H); 1.89 (t, J=7.2 Hz, 2H).

### Example 26: Preparation of 3-{[2-fluoro-3-(methylaminosulfonylamino)phenyl]methyl}-7-(2-oxo-1-pyridyl)-3,4-dihydro-2H-1,3-benzoxazin-2-one (E-26; Compound 139)

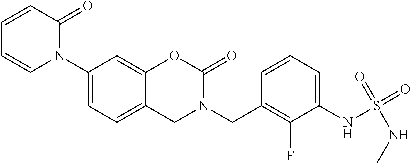 [SMILES: CNS(=O)(=O)Nc1cccc(CN2Cc3ccc(-n4ccccc4=O)cc3OC2=O)c1F]

E-26 was synthesized by following similar synthesis sequence and experimental procedures as described for the synthesis of E-17, using key intermediates I-13 and I-31. LCMS (ES) m/z: 459.3 |M+H|⁺. ¹H NMR (400 MHz, DMSO-d₆): δ 9.43 (s, 1H); 7.63 (d, J=4.0 Hz, 1H); 7.53-7.49 (m, 1H); 7.42-7.36 (m, 2H); 7.23-7.15 (m, 5H); 6.48 (d, J=8.0 Hz, 1H); 6.34-6.30 (m, 1H); 4.70 (s, 2H); 4.56 (s, 2H); 2.50 (d, J=4.4 Hz, 3H).

### Example 27: Preparation of 3-({2-fluoro-3-[(methylsulfamoyl)amino]phenyl}methyl)-7-(pyrimidin-2-yloxy)-3,4-dihydro-2H-1,3-benzoxazin-2-one (E-27; Compound 99)

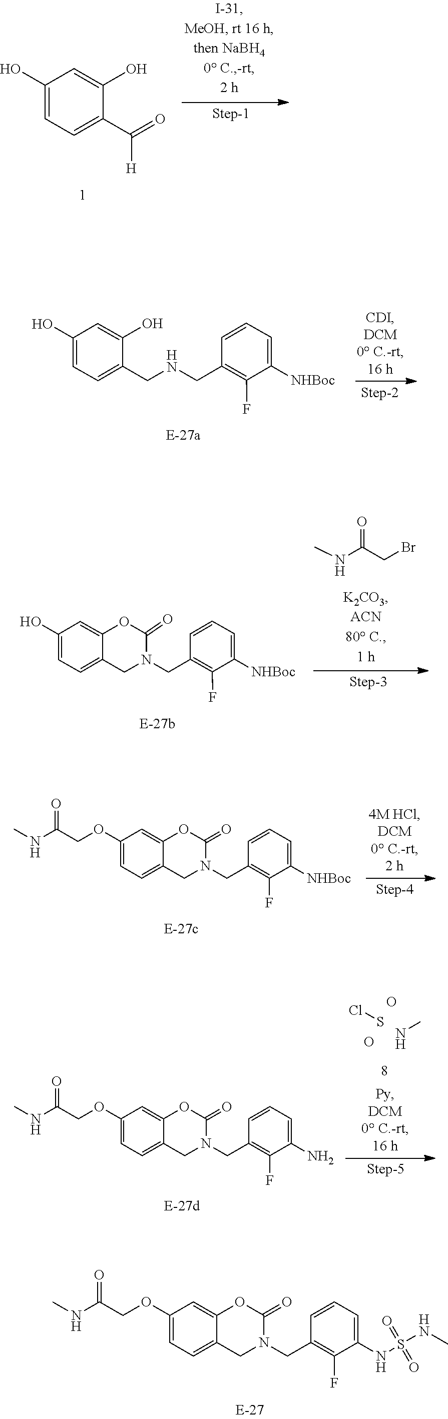

Step-1: Synthesis of tert-butyl (3-(((2,4-dihydroxybenzyl)amino)methyl)-2-fluorophenyl)carbamate (E-27a): Compound E-27a (5.1 g) was synthesized by following procedure as described for the synthesis of E-01 (step-1) using 2,4-dihydroxybenzaldehyde (1) (2.0 g, 14.5 mmol) and I-31 (3.83 g, 15.9 mmol) as the starting materials. LCMS (ES) m/z: 363.2 ┌M+H┐⁺.

Step-2: Synthesis of tert-butyl (2-NSuoro-3-((7-hydroxy-2-oxo-2H-benzo[e][1,3]oxazin-3(4H)-yl)methyl)phenyl)carbamate (E-27b): Compound E-27b (3.0 g) was synthesized by following procedure as described for the synthesis of E-01 (step-2) using tert-butyl (3-(((2,4-dihydroxybenzyl)amino)methyl)-2-fluorophenyl)carbamate (E-27a) (5.0 g, 13.8 mmol) as the starting materials. LCMS (ES) m/z: 333.1 [(M-^{t}Bu)+H]⁺.

Step-3: Synthesis of tert-butyl (2-fluoro-3-((7-(2-(methylamino)-2-oxoethoxy)-2-oxo-2H-benzo[e][1,3]oxazin-3(4H)-yl)methyl)phenyl)carbamate (E-27c): To a stirred solution of tert-butyl N-{2-fluoro-3-[(7-hydroxy-2-oxo-3,4-dihydro-2H-1,3-benzoxazin-3-yl)methyl]phenyl}carbamate (E-27b) (250 mg, 644 μmol) in ACN (5 mL) was added K₂CO₃ (222 mg, 1.61 mmol) and 2-chloro-N-methylacetamide (138 mg, 1.29 mmol) at room temperature. The reaction mixture was then stirred at 80° C., while monitoring reaction progress by TLC. After complete consumption of starting material (1 h), the reaction mixture was cooled to room temperature and filtered through celite. The filtrate was concentrated under reduced pressure and the residue was purified by Combi-Flash (using gradient eluent of 0-50% of EtOAc in heptane) to afford desired compound E-27c (0.2 g) as an off-white solid. LCMS (ES) m/z: 458.3 [M−H]⁺.

Step-4: Synthesis of 2-((3-(3-amino-2-fluorobenzyl)-2-oxo-3,4-dihydro-2H-benzo[e][1,3]oxazin-7-yl)oxy)-N-methylacetamide (E-27d): Compound E-27d (0.2 g) was synthesized by following procedure as described for the synthesis of E-17 (step-3) using tert-butyl (2-fluoro-3-((7-(2-(methylamino)-2-oxoethoxy)-2-oxo-2H-benzo[e][1,3]oxazin-3(4H)-yl)methyl)phenyl)carbamate (E-27c) (0.230 g, 501 mol) as the starting material. LCMS (ES) m/z: 360.1 [M+H]⁺.

Step-5: Synthesis of 2-((3-(2-fluoro-3-((N-methylsulfamoyl)amino)benzyl)-2-oxo-3,4-dihydro-2H-benzo[e][1,3]oxazin-7-yl)oxy)-N-methylacetamide (E-27): Example E-27 (0.060 g) was synthesized by following procedure as described for the synthesis of E-17 (step-4) using 2-({3-[(3-amino-2-fluorophenyl)methyl]-2-oxo-3,4-dihydro-2H-1,3-benzoxazin-7-yl}oxy)-N-methylacetamide (E-27d) (0.2 g, 0.557 mmol) as the starting material. LCMS (ES) m/z: 451.2 [M−H]⁺.; ¹H NMR (400 MHz, DMSO-d₆): δ 9.43 (s, 1H); 8.04 (s, 1H); 7.40-7.36 (m, 1H); 7.23 (br s, 1H); 7.16-7.13 (m, 3H); 6.75 (dd, J=8.4, 2.4 Hz, 1H); 6.66 (d, J=2.4 Hz, 1H); 4.65 (s, 2H); 4.47 (s, 2H); 4.41 (s, 2H); 2.64 (d, J=4.8 Hz, 3H); 2.50 (d, J=4.4 Hz, 3H).

### Example 28: Preparation of 6-fluoro-3-(2-fluoro-3-((N-methylsulfamoyl)amino)benzyl)-2-oxo-3,4-dihydro-2H-benzo[e][1,3]oxazin-7-yl dimethylcarbamate (E-28; Compound 120)

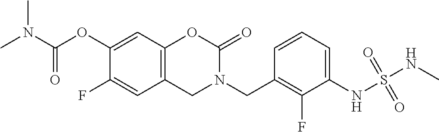

E-28 was synthesized by following similar synthesis sequence and experimental procedures as described for the synthesis of E-27, using key intermediate I-31 and I-05. LCMS (ES) m/z: 471.3 [M+H]⁺. ¹H NMR (400 MHz, DMSO-d₆): δ 9.41 (s, 1H); 7.42-7.36 (m, 1H); 7.29 (d, J=10.0 Hz, 1H); 7.22-7.08 (m, 4H); 4.66 (s, 2H); 4.46 (s, 2H); 3.04 (s, 3H); 2.91 (s, 3H); 2.53 (d, J=4.4 Hz, 3H).

### Example 29: Preparation of 6,8-difluoro-3-(2-fluoro-3-((N-methylsulfamoyl)amino)benzyl)-2-oxo-3,4-dihydro-2H-benzo[e][1,3]oxazin-7-yl dimethylcarbamate (E-29; Compound 96)

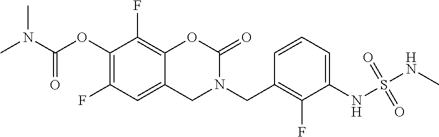

E-29 was synthesized by following similar synthesis sequence and experimental procedures as described for the synthesis of E-27, using key intermediates I-03 and I-31. LCMS (ES) m/z: 489.3 [M+H]⁺. ¹H NMR (400 MHz, DMSO-d₆): δ 9.41 (s, 1H); 7.44-7.34 (m, 1H); 7.22-7.14 (m, 4H); 4.67 (s, 2H); 4.52 (s, 2H); 3.08 (s, 3H); 2.94 (s, 3H); 2.50 (d, J=4.4 Hz, 3H).

### Example 30: Preparation of 8-fluoro-3-{[2-fluoro-3-(methylaminosulfonylamino)phenyl]methyl}-7-(1,3-oxazol-2-yloxy)-3,4-dihydro-2H-1,3-benzoxazin-2-one (E-30; Compound 91)

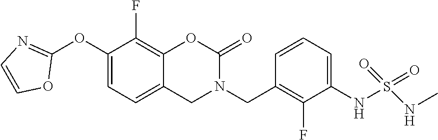 [SMILES: CNS(=O)(=O)Nc1cccc(CN2Cc3ccc(Oc4ncco4)c(F)c3OC2=O)c1F]

E-30 was synthesized by following similar synthesis sequence and experimental procedures as described for the synthesis of E-27, using key intermediates 4 and I-31. LCMS (ES) m/z: 467.3 [M+H]⁺. ¹H NMR (400 MHz, DMSO-d₆): δ 9.42 (s, 1H); 7.86 (d, J=0.8 Hz 1H); 7.41-7.33 (m, 2H); 7.24-7.13 (m, 4H); 6.99 (s, 1H); 4.68 (s, 2H); 4.56 (s, 2H); 2.52 (d, J=4.4 Hz, 3H).

### Example 31: Preparation of 6-fluoro-3-{[2-fluoro-3-(methylaminosulfonylamino)phenyl]methyl}-7-(2-pyrimidinyloxy)-3,4-dihydro-2H-1,3-benzoxazin-2-one (E-31; Compound 83)

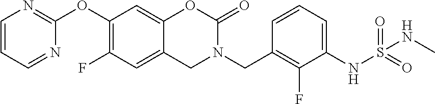 [SMILES: CNS(=O)(=O)Nc1cccc(CN2Cc3cc(F)c(Oc4ncccn4)cc3OC2=O)c1F]

E-31 was synthesized by following similar synthesis sequence and experimental procedures as described for the synthesis of E-27, using key intermediates I-05 and I-31. LCMS (ES) m/z: 478.3 [M+H]⁺. ¹H NMR (400 MHz, DMSO-d₆): δ 9.43 (s, 1H); 8.68-8.66 (m, 2H); 7.42-7.32 (m, 3H); 7.25 (d, J=6.8 Hz, 1H); 7.20-7.10 (m, 3H); 4.68 (s, 2H); 4.53 (s, 2H); 2.52 (d, J=4.4 Hz, 3H).

### Example 32: Preparation of 3-({2-fluoro-3-[(methylsulfamoyl)amino]phenyl}methyl)-7-(1,2,4-triazin-3-yloxy)-3,4-dihydro(4,4-² H₂)-2H-1,3-benzoxazin-2-one (E-32; Compound 31)

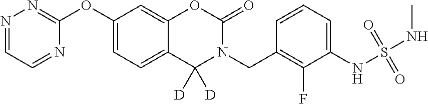 [SMILES: [2H]C1([2H])c2ccc(Oc3nccnn3)cc2OC(=O)N1Cc1cccc(NS(=O)(=O)NC)c1F]

E-32 was synthesized by following similar synthesis sequence and experimental procedures as described for the synthesis of E-27, using key intermediates I-16 and I-31 (note: NaBD₄ was used as reducing agent in step-1 while reductive amination). LCMS (ES) m/z: 463.3 [M+H]⁺. ¹H NMR (400 MHz, DMSO-d₆): δ 9.28 (s, 1H); 9.27 (s, 1H); 8.74 (d, J=2.4 Hz, 1H); 7.43-7.37 (m, 1H); 7.34 (d, J=8.4 Hz, 1H); 7.24-7.08 (m, 5H); 4.68 (s, 2H); 2.52 (d, J=4.4 Hz, 3H).

### Example 33: Preparation of 8-fluoro-3-(2-fluoro-3-((N-methylsulfamoyl)amino)benzyl)-2-oxo-3,4-dihydro-2H-benzo[e][1,3]oxazin-7-yl dimethylcarbamate (E-02; Compound 14)

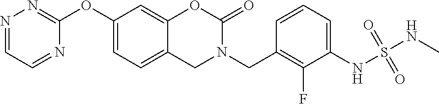 [SMILES: CNS(=O)(=O)Nc1cccc(CN2Cc3ccc(Oc4nccnn4)cc3OC2=O)c1F]

E-33 was synthesized by following similar synthesis sequence and experimental procedures as described for the synthesis of E-27, using key intermediate E-01c and 3-(methylsulfonyl)-1,2,4-triazine as the starting materials. LCMS (ES) m/z: 461.3 [M+H]⁺. ¹H NMR (400 MHz, DMSO-d₆): δ 9.43 (s, 1H); 9.27 (d, J=2.4 Hz, 1H), 8.74 (d, J=2.4 Hz, 1H); 7.42-7.36 (m, 1H); 7.34 (d, J=8.0 Hz 1H); 7.14-7.04 (m, 5H); 4.68 (s, 2H), 4.52 (s, 2H); 2.66 (d, J=4.4 Hz, 3H).

### Example 34: Preparation of 3-({2-fluoro-3-[(methylsulfamoyl)amino]phenyl}methyl)-7-(pyrimidin-2-yloxy)-3,4-dihydro-2H-1,3-benzoxazin-2-one (E-34; Compound 105)

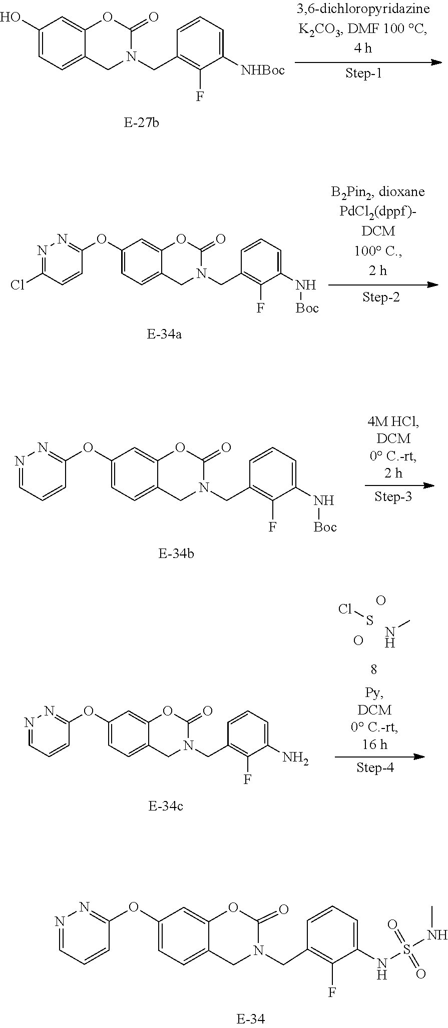

Step-1: Synthesis of tert-butyl (3-((7-((6-chloropyridazin-3-yl)oxy)-2-oxo-2H-benzo[e][1,3]oxazin-3(4H)-yl)methyl)-2-fluorophenyl)carbamate (E-34a): To a stirred solution of tert-butyl N-{2-fluoro-3-[(7-hydroxy-2-oxo-3,4-dihydro-2H-1,3-benzoxazin-3-yl)methyl]phenyl}carbamate (E-27b) (3 g, 7.72 mmol) in DMF (30 mL) was added K₂CO₃(2.14 g, 15.4 mmol) and 3,6-dichloropyridazine (2.3 g, 15.4 mmol). The reaction mixture was then stirred at 100° C., while monitoring reaction progress by TLC. After complete consumption of starting material (4 h), the reaction mixture was cooled to room temperature and cold water (30 mL) was added to it. Extraction was carried out using EtOAc (3×50 mL); the combined organic extracts were washed with brine solution (2×50 mL), dried over anhydrous Na₂SO₄, filtered and concentrated under reduced pressure. The residue was purified by Combi-Flash (using gradient elution of 0-70% of EtOAc in heptane) to afford the desired compound E-34a (2.3 g) as a pale yellow solid. LCMS (ES) m/z: 445.2 [(M-^{t}Bu)+H]⁺.

Step-2: Synthesis of tert-butyl (2-fluoro-3-((2-oxo-7-(pyridazin-3-yloxy)-2H-benzo[e][1,3]oxazin-3(4H)-yl)methyl)phenyl)carbamate (E-34b): To a stirred solution of tert-butyl N-[3-({7-[(6-chloropyridazin-3-yl)oxy]-2-oxo-3,4-dihydro-2H-1,3-benzoxazin-3-yl}methyl)-2-fluorophenyl]carbamate (E-34a) (1.5 g, 2.99 mmol) in 1,4-dioxane (20 mL) was added KOAc (882 mg, 8.98 mmol) and bis(pinacolato)diboron (836 mg, 3.29 mmol). Argon gas was then purged through it for 5 min before addition of Pd(dppf)Cl₂.DCM (245 mg, 299 mol). The reaction mixture was then stirred at 100° C., while monitoring reaction progress by TLC. After complete consumption of starting material (3 h), the reaction mixture was cooled to room temperature, filtered through celite pad and washed with EtOAc (2×30 mL). The filtrate was washed with water (2×50 mL), brine solution (2×50 mL), dried over anhydrous Na₂SO₄, filtered and concentrated under reduced pressure. The residue was purified by Combi-Flash (using gradient elution of 0-70% of EtOAc in heptane) to afford desired compound E-34b (0.8 g) as a pale yellow solid. LCMS (ES) m/z: 467.1 [M+H]⁺.

Step-3: Synthesis of 3-(3-amino-2-fluorobenzyl)-7-(pyridazin-3-yloxy)-3,4-dihydro-2H-benzo[e][1,3]oxazin-2-one (E-34c): Compound E-34c (0.76 g) was synthesized by following procedure as described for the synthesis of E-17 (step-3) using tert-butyl (2-fluoro-3-((2-oxo-7-(pyridazin-3-yloxy)-2H-benzo[e][1,3]oxazin-3(4H)-yl)methyl)phenyl)carbamate (E-34b) (1.2 g, 2.57 mmol) as the starting material. LCMS (ES) m/z: 367.3 [M+H]⁺.

Step-4: 3-({2-fluoro-3-[(methylsulfamoyl)amino]phenyl}methyl)-7-(pyridazin-3-yloxy)-3,4-dihydro-2H-1,3-benzoxazin-2-one (E-42): Example 34 (0.940 g) was synthesized by following procedure as described for the synthesis of E-17 (step-4) using 3-(3-amino-2-fluorobenzyl)-7-(pyridazin-3-yloxy)-3,4-dihydro-2H-benzo[e][1,3]oxazin-2-one (E-34c) (1.0 g, 2.73 mmol) as the starting material. The final compound was purified by reverse phase preparative HPLC purification [using Sunfire-C18 (19 mm×250 mm, 5 m particle size) column, water and acetonitrile as mobile phase with 19.0 mL/min flow rate]. LCMS (ES) m/z: 460.1 [M+H]⁺. ¹H NMR (400 MHz, DMSO d₆): δ 9.42 (s, 1H); 9.03 (d, J=1.2 Hz, 1H); 7.82-7.76 (m, 1H); 7.49 (dd, J₁=1.2 Hz, J₂=8.8 Hz, 1H); 7.44-7.36 (m, 1H); 7.31 (d, J=8.4 Hz, 1H); 7.24-7.16 (m, 3H); 7.04-6.98 (m, 2H); 4.69 (s, 2H); 4.52 (s, 1H); 2.53 (d, J=4.8 Hz, 3H).

### Example 35: Preparation of 3-{[2-fluoro-3-(methylaminosulfonylamino)phenyl]methyl}-7-(3-pyridazinyloxy)(4,4-²H₂)-3,4-dihydro-2H-1,3-benzoxazin-2-one (E-35; Compound 52)

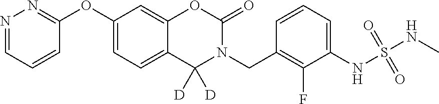 [SMILES: [2H]C1([2H])c2ccc(Oc3cccnn3)cc2OC(=O)N1Cc1cccc(NS(=O)(=O)NC)c1F]

E-35 was synthesized by following similar synthesis sequence and experimental procedures as described for the synthesis of E-34, using key intermediates I-16 and I-31. LCMS (ES) m/z: 462.3 [M+H]⁺. 1′H NMR (400 MHz, DMSO-d₆): δ 9.44 (s, 1H); 9.03-9.01 (m, 1H); 7.80-7.76 (m, 1H); 7.48 (dd, J₁=0.8 Hz, J₂=8.8 Hz, 1H); 7.41-7.37 (m, 1H); 7.31 (d, J=8.0 Hz, 1H); 7.22-7.12 (m, 3H); 7.02-6.99 (m, 2H); 4.68 (s, 2H); 2.53 (d, J=4.8 Hz, 3H).

### Example 36: Preparation of 3-{[3-(dimethylaminosulfonylamino)-2-fluorophenyl]methyl}-7-(3-pyridazinyloxy)(4,4-²H₂)-3,4-dihydro-2H-1,3-benzoxazin-2-one (E-36; Compound 39)

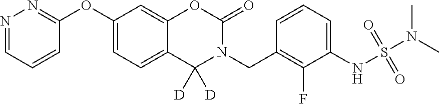 [SMILES: [2H]C1([2H])c2ccc(Oc3cccnn3)cc2OC(=O)N1Cc1cccc(NS(=O)(=O)N(C)C)c1F]

E-36 was synthesized by following similar synthesis sequence and experimental procedures as described for the synthesis of E-34, using key intermediates I-16 and I-31. (note: dimethylsulfamoyl chloride was used in the last step). LCMS (ES) m/z: 476.3 [M+H]⁺. 1H NMR (400 MHz, DMSO-d₆): δ 9.63 (s, 1H); 9.02 (dd, J₁=1.2 Hz, J₂=4.4 Hz, 1H); 7.80-7.76 (m, 1H); 7.48 (dd, J₁=1.2 Hz, J₂=9.2 Hz, 1H); 7.42-7.38 (m, 1H); 7.31 (d, J=8.0 Hz, 1H); 7.22-7.12 (m, 2H); 7.02-6.98 (m, 2H); 4.69 (s, 2H); 2.68 (s, 6H).

### Example 37: Preparation of 3-{[3-(dimethylaminosulfonylamino)-2-fluorophenyl]methyl}-7-(3-pyridazinyloxy)-3,4-dihydro-2H-1,3-benzoxazin-2-one (E-37; Compound 37)

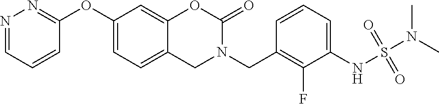 [SMILES: CN(C)S(=O)(=O)Nc1cccc(CN2Cc3ccc(Oc4cccnn4)cc3OC2=O)c1F]

E-37 was synthesized by following synthesis sequence and procedures as described for the synthesis of E-34, using intermediate E-34c and dimethylsulfamoyl chloride as the starting materials. LCMS (ES) m/z: 474.3 [M+H]⁺. ¹H NMR (400 MHz, DMSO-d₆): δ 9.62 (s, 1H); 9.01 (d, J=4.4 Hz, 1H); 7.79-7.76 (m, 1H); 7.48 (d, J=9.6 Hz, 1H); 7.40 (t, J=8.0 Hz, 1H); 7.31 (d, J=8.0 Hz, 1H); 7.23-7.14 (m, 2H); 6.99 (s, 2H); 4.51 (s, 2H); 4.72 (s, 2H); 2.72 (s, 6H).

### Example 38: Preparation of N-methoxycarbonyl(2-fluoro-3-{[2-oxo-7-(3-pyridazinyloxy)-3,4-dihydro-2H-1,3-benzoxazin-3-yl]methyl}phenyl)amine (E-38; Compound 11)

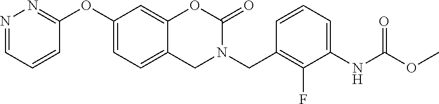 [SMILES: COC(=O)Nc1cccc(CN2Cc3ccc(Oc4cccnn4)cc3OC2=O)c1F]

E-38 was synthesized by following similar synthesis sequence and experimental procedures as described for the synthesis of E-34, using key intermediates E-34c and methylchloroformate as the starting materials. LCMS (ES) m/z: 425.3 [M+H]⁺. ¹H NMR (400 MHz, DMSO-d₆): δ 9.34 (s, 1H); 9.02 (dd, J₁=1.2 Hz, J₂=4.8 Hz, 1H); 7.80-7.76 (m, 1H); 7.65-7.60 (m, 1H); 7.48 (dd, J₁=1.2 Hz, J₂=7.6 Hz, 1H); 7.31 (d, J=8.4, 1H); 7.18-7.12 (m, 2H); 7.02-6.98 (m, 2H); 4.69 (s, 2H), 4.51 (s, 2H); 3.67 (s, 3H).

### Example 39: Preparation of 3-{[3-fluoro-2-(methylaminosulfonylamino)-4-pyridyl]methyl}-7-(3-pyridazinyloxy)-3,4-dihydro-2H-1,3-benzoxazin-2-one (E-39; Compound 1)

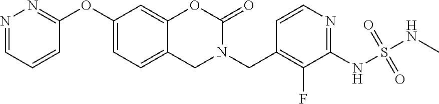 [SMILES: CNS(=O)(=O)Nc1nccc(CN2Cc3ccc(Oc4cccnn4)cc3OC2=O)c1F]

E-39 was synthesized by following similar synthesis sequence and experimental procedures as described for the synthesis of E-34, using intermediate I-38 and 2,4-dihydroxybenzaldehyde as the starting materials. LCMS (ES) m/z: 461.2 [M+H]⁺. ¹H NMR (400 MHz, DMSO-d₆): δ 10.43 (s, 1H); 9.02 (dd, J₁=1.2 Hz, J, =4.4 Hz, 1H); 8.07 (d, J=4.8 Hz, 1H); 7.81-7.77 (m, 1H); 7.50 (dd, J₁=1.2 Hz, J₂=9.2 Hz, 1H); 7.33 (d, J=8.4 Hz, 1H); 7.10-7.00 (m, 4H); 4.71 (s, 2H); 4.59 (s, 2H); 2.53 (d, J=4.8 Hz, 3H).

### Example 40: Preparation of 3-(2-fluoro-3-((N-methylsulfamoyl)amino)benzyl)-2-oxo-6-(trifluoromethyl)-3,4-dihydro-2H-benzo[e][1,3]oxazin-7-yl dimethylcarbamate (E-40; Compound 97)

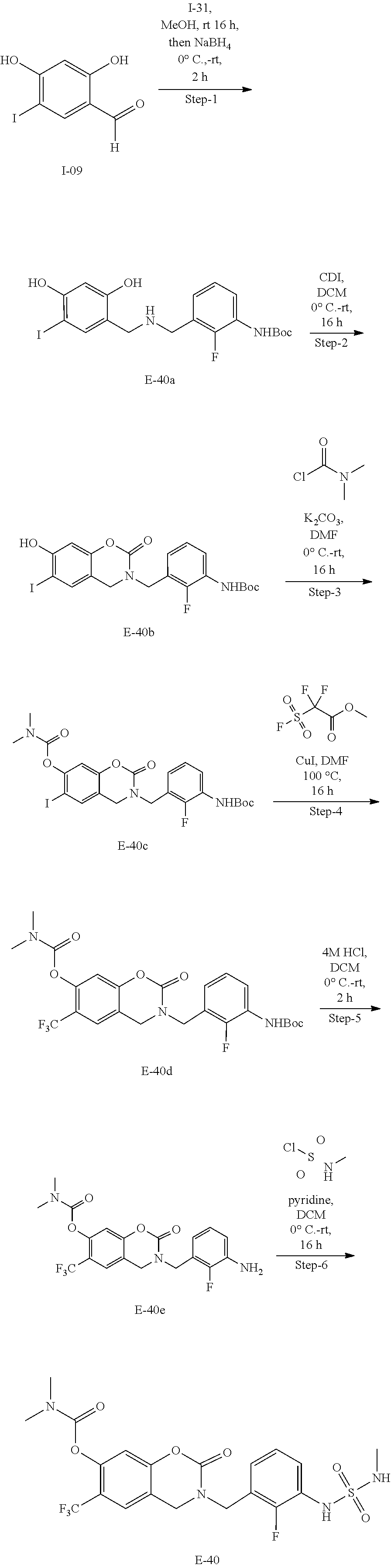

Step-1: Synthesis of tert-butyl (3-(((2,4-dihydroxy-5-iodobenzyl)amino)methyl)-2-fluorophenyl)carbamate (E-40a): Compound E-40a (0.8 g) was synthesized by following procedure as described for the synthesis of E-01 (step-1) using 2,4-dihydroxy-5-iodobenzaldehyde (I-09) (1 g, 3.79 mmol) and tert-butyl N-[3-(aminomethyl)-2-fluorophenyl]carbamate (I-31) (0.910 g, 3.79 mmol) as the starting materials. LCMS (ES) m/z: 489.1 [M+H]⁺.

Step-2: Synthesis of tert-butyl (2-fluoro-3-((7-hydroxy-6-iodo-2-oxo-2H-benzo[e][1,3]oxazin-3(4H)-yl)methyl)phenyl)carbamate (E-40b): Compound E-40b (0.2 g) was synthesized by following procedure as described for the synthesis of E-01 (step-2) using tert-butyl N-[3-({[(2,4-dihydroxy-5-iodophenyl)methyl]amino}methyl)-2-fluorophenyl]carbamate (E-40a) (0.8 g, 1.64 mmol) as the starting material. LCMS (ES) m/z: 513.1 [M−H]⁺.

Step-3: Synthesis of 3-(3-((tert-butoxycarbonyl)amino)-2-fluorobenzyl)-6-iodo-2-oxo-3,4-dihydro-2H-benzo[e][1,3]oxazin-7-yl dimethylcarbamate (E-40c): Compound E-40c (0.190 g) was synthesized by following procedure as described for the synthesis of E-01 (step-4) using tert-butyl N-{2-fluoro-3-[(7-hydroxy-6-iodo-2-oxo-3,4-dihydro-2H-1,3-benzoxazin-3-yl)methyl]phenyl}carbamate (E-40b) (0.2 g, 389 μmol) as the starting material. LCMS (ES) m/z: 584.1 [M−H]⁺.

Step-4: Synthesis of 3-(3-((tert-butoxycarbonyl)amino)-2-fluorobenzyl)-2-oxo-6-(trifluoromethyl)-3,4-dihydro-2H-benzo[e][1,3]oxazin-7-yl dimethylcarbamate (E-40d): To a stirred solution of 3-[(3-{[(tert-butoxy)carbonyl]amino]-2-fluorophenyl)methyl]-6-iodo-2-oxo-3,4-dihydro-2H-1,3-benzoxazin-7-yl N,N-dimethylcarbamate (E-40c) (150 mg, 256 μmol) in DMF (3 mL) was added methyl 2,2-difluoro-2-(fluorosulfonyl)acetate (164 μL, 1.28 mmol) and CuI (146 mg, 769 μmol). The reaction mixture was then stirred at 100° C., while monitoring reaction progress by TLC. After completion the reaction (16 h), the reaction mixture was cooled to room temperature and water (20 mL) was added to it. Extraction was carried out using EtOAc (30 mL×2); the combined organic extracts were washed with brine solution (30 mL), dried over anhydrous Na₂SO₄, filtered and concentrated under reduced pressure. The residue was purified by Combi-Flash (using gradient elution of 0-50% EtOAc in heptane) to afford desired compound E-40d (120 mg) as a white solid. LCMS (ES) m/z: 526.37 [M−H]⁺.

Step-5: Synthesis of 3-(3-amino-2-fluorobenzyl)-2-oxo-6-(trifluoromethyl)-3,4-dihydro-2H-benzo[e][1,3]oxazin-7-yl dimethylcarbamate (E-40e): Compound E-40e (0.1 g) was synthesized by following procedure as described for the synthesis of E-17 (step-3) using 3-[(3-{[(tert-butoxy)carbonyl]amino}-2-fluorophenyl)methyl]-2-oxo-6-(trifluoromethyl)-3,4-dihydro-2H-1,3-benzoxazin-7-yl N,N-dimethylcarbamate (E-40d) (120 mg, 227 μmol) as the starting material. LCMS (ES) m/z: 428.0 [M+H]⁺.

Step-6: Synthesis of 3-(2-fluoro-3-((N-methylsulfamoyl)amino)benzyl)-2-oxo-6-(trifluoromethyl)-3,4-dihydro-2H-benzo[e][1,3]oxazin-7-yl dimethylcarbamate (E-40): Example E-40 (0.007 g) was synthesized by following procedure as described for the synthesis of E-17 (step-4) using 3-[(3-amino-2-fluorophenyl)methyl]-2-oxo-6-(trifluoromethyl)-3,4-dihydro-2H-1,3-benzoxazin-7-yl N,N-dimethylcarbamate (E-40e) (0.1 g, 234 mol) as the starting material. The final compound was purified by reverse phase preparative HPLC purification [using Xbridge C₈ (19 mm×250 mm, 5 m particle size) column, ammonium bicarbonate in water and acetonitrile as mobile phase]. LCMS (ES) m/z: 519.15 [M−H]⁺. ¹H NMR (400 MHz, DMSO-d₆): δ 9.41 (br s, 1H); 7.71 (s, 1H); 7.42-7.37 (m, 1H); 7.24-7.12 (m, 4H); 4.67 (s, 2H); 4.54 (s, 2H); 3.03 (s, 3H); 2.92 (s, 3H); 2.50 (d, J=4.4 Hz, 3H).

### Example 41: Preparation of 3-({3-[(methylsulfamoyl)amino]phenyl}methyl)-7-(pyrimidin-2-yloxy)-3,4-dihydro-2H-1,3-benzoxazin-2-one (E-41; Compound 159)

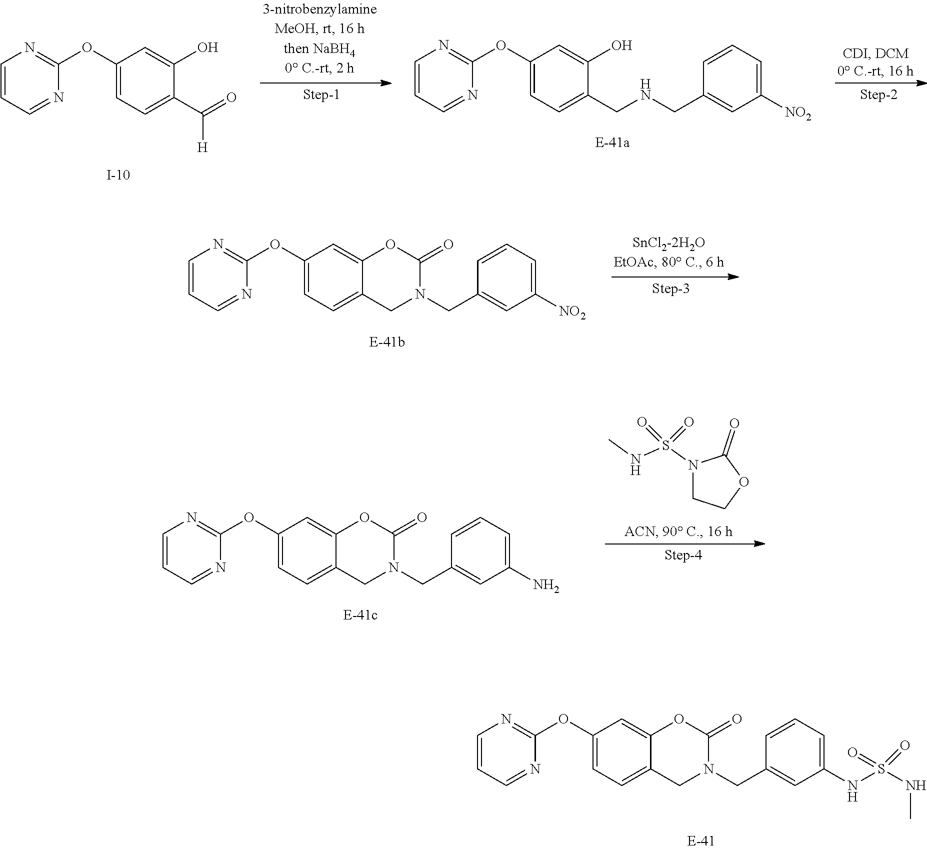

Step-1: Synthesis of 2-(((3-nitrobenzyl)amino)methyl)-5-(pyrimidin-2-yloxy)phenol (E-41a): Compound E-41a (2.6 g) was synthesized by following procedure as described for the synthesis of E-01 (step-1) using 2-hydroxy-4-(pyrimidin-2-yloxy)benzaldehyde (I-10) (0.3 g, 1.39 mmol) and 1-(3-nitrophenyl)methanamine (211 mg, 1.39 mmol) as the starting materials. LCMS (ES) m/z: 353.1 [M+H]⁺.

Step-2: Synthesis of 3-(3-nitrobenzyl)-7-(pyrimidin-2-yloxy)-3,4-dihydro-2H-benzo[e][1,3]oxazin-2-one (E-41b): Compound E-41b (0.386 g) was synthesized by following procedure as described for the synthesis of E-01 (step-2) using 2-({[(3-nitrophenyl)methyl]amino}methyl)-5-(pyrimidin-2-yloxy)phenol (E-41a) (0.4 g, 1.14 mmol) as the starting material. LCMS (ES) m/z: 379.1 [M+H]⁺.

Step-3: Synthesis of 3-(3-aminobenzyl)-7-(pyrimidin-2-yloxy)-3,4-dihydro-2H-benzo[e][1,3]oxazin-2-one (E-41c): To a stirred solution of 3-[(3-nitrophenyl)methyl]-7-(pyrimidin-2-yloxy)-3,4-dihydro-2H-1,3-benzoxazin-2-one (E-41b) (0.1 g, 264 μmol) in EtOAc (10 mL) was added SnCl₂·2H₂O (417 mg, 1.85 mmol) at room temperature and the reaction mixture was stirred at 80° C., while monitoring reaction progress by TLC/LCMS. After complete consumption of starting material (6 h), the reaction mixture was cooled to room temperature and quenched with addition of saturated NaHCO₃ solution (15 mL). Extraction was carried out using EtOAc (2×20 mL); the combined organic extracts were washed with brine solution (2×10 mL), dried over anhydrous Na₂SO₄, filtered and concentrated under reduced pressure. The residue was purified by Combi-Flash (using gradient elution of 0-10% of MeOH in DCM) to afford the desired compound E-41c (62 mg) as a pale yellow thick oil. LCMS (ES) m/z: 349.2 [M+H]⁺.

Step-4: Synthesis of 3-({3-[(methylsulfamoyl)amino]phenyl}methyl)-7-(pyrimidin-2-yloxy)-3,4-dihydro-2H-1,3-benzoxazin-2-one (E-41): To a stirred solution of 3-(3-aminophenyl)methyl]-7-(pyrazin-2-yloxy)-3,4-dihydro-2H-1,3-benzoxazin-2-one (E-41c) (180 mg, 517 μmol) in ACN (15 mL) was added TEA (212 μL, 1.55 mmol) and N-methyl-2-oxo-1,3-oxazolidine-3-sulfonamide (186 mg, 1.03 mmol). The reaction mixture was then stirred at 90° C., while monitoring reaction progress by TLC/LCMS. After complete consumption of starting material (16 h), the reaction mixture was cooled to room temperature and volatiles were removed under reduced pressure. The residue was purified by Combi-Flash (using gradient elution of 0-6% of MeOH in DCM) followed by reverse phase preparative HPLC purification [using X Terra C18 (250 mm×19 mm, 5 m particle size) column, 0.1% ammonia in water and acetonitrile as mobile phase with 19 mL/min flow rate] to afford desired compound E-41 (145 mg) as an off-white solid. LCMS (ES) m/z: 442.2 [M+H]⁺. ¹H NMR (400 MHz, DMSO-d₆): δ 9.77 (s, 1H); 8.76 (d, J=4.4 Hz, 2H); 7.42-7.37 (m, 4H); 7.26 (d, J=7.6 Hz, 2H); 7.11-7.08 (m, 3H); 4.70 (s, 2H); 4.56 (s, 2H); 2.56 (d, J=5.2 Hz, 3H).

### Example 42: Preparation of 3-(3-((N-methylsulfamoyl)amino)benzyl)-2-oxo-3,4-dihydro-2H-benzo[e][1,3]oxazin-7-yl dimethylcarbamate (E-42; Compound 158)

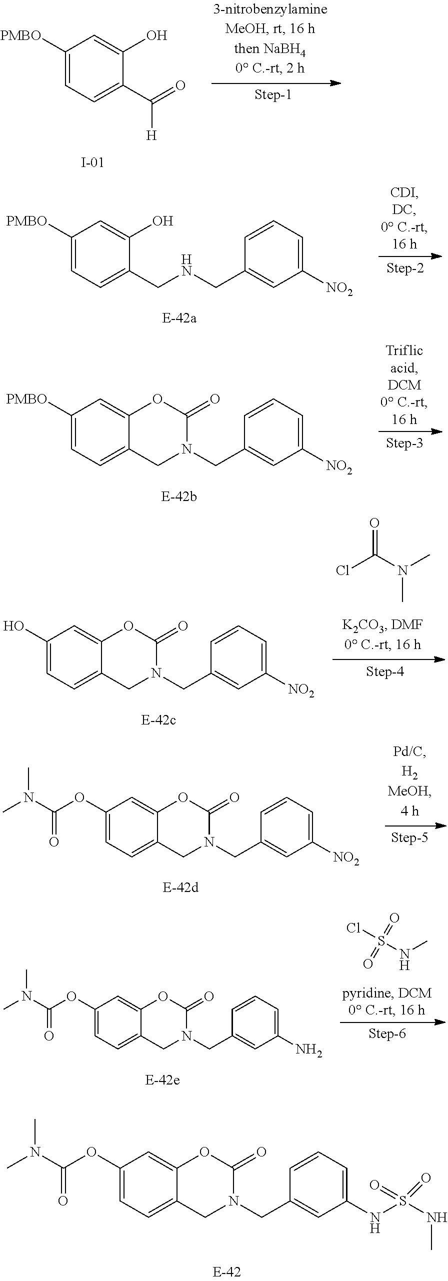

Step-1: Synthesis of 5-((4-methoxybenzyl)oxy)-2-(((3-nitrobenzyl)amino)methyl)phenol (E-42a): Compound E-42a (1.1 g) was synthesized by following procedure as described for the synthesis of E-01 (step-1) using 2-hydroxy-4-[(4-methoxyphenyl)methoxy]benzaldehyde (I-01) (1 g, 3.87 mmol) and 1-(3-nitrophenyl)methanamine (589 mg, 3.87 mmol) as the starting materials. LCMS (ES) m/z: 395 [M+H]⁺.

Step-2: Synthesis of 7-((4-methoxybenzyl)oxy)-3-(3-nitrobenzyl)-3,4-dihydro-2H-benzo[e][1,3]oxazin-2-one (E-42b): Compound E-42b (0.822 g) was synthesized by following procedure as described for the synthesis of E-01 (step-2) using 5-[(4-methoxyphenyl)methoxy]-2-({[(3-nitrophenyl)methyl]amino}methyl)phenol (E-42a) (1 g, 2.54 mmol) as the starting material. LCMS (ES) m/z: 421 [M+H]⁺.

Step-3: Synthesis of 7-hydroxy-3-(3-nitrobenzyl)-3,4-dihydro-2H-benzo[e][1,3]oxazin-2-one (E-42c): To a stirred solution of 7-[(4-methoxyphenyl)methoxy]-3-[(3-nitrophenyl)methyl]-3,4-dihydro-2H-1,3-benzoxazin-2-one (E-42b) (0.9 g, 2.14 mmol) in DCM (30 mL) was added trifluoromethanesulfonic acid (379 μL, 4.28 mmol) at 0° C. and the reaction mixture was stirred at room temperature, while monitoring reaction progress by TLC/LCMS. After complete consumption of starting material (16 h), the reaction mixture was quenched with saturated NaHCO₃ solution (30 mL) at 0° C. and extraction was carried out using DCM (2×30 mL). The combined organic extracts were washed with water (2×20 mL), brine solution (2×20 mL), dried over anhydrous Na₂SO₄, filtered and concentrated under reduced pressure. The residue was purified by Combi-Flash (using gradient elution of 0-60% of EtOAc in heptane) to afford the desired E-42c (520 mg) as a pale yellow solid. LCMS (ES) m/z: 301.1 (M+H)⁺.

Step-4: Synthesis of 3-(3-nitrobenzyl)-2-oxo-3,4-dihydro-2H-benzo[e][1,3]oxazin-7-yl dimethylcarbamate (E-42d): Compound E-42d (0.450 g) was synthesized by following procedure as described for the synthesis of E-01 (step-4) using tert-butyl N-[(4-aminophenyl)methyl]carbamate (E-42c) (0.5 g, 1.67 mmol) as the starting material. LCMS (ES) m/z: 372.2 [M+H]⁺.

Step-5: Synthesis of 3-(3-aminobenzyl)-2-oxo-3,4-dihydro-2H-benzo[e][1,3]oxazin-7-yl dimethylcarbamate (E-42e): To a stirred solution of 3-[(3-nitrophenyl)methyl]-2-oxo-3,4-dihydro-2H-1,3-benzoxazin-7-yl N,N-dimethylcarbamate (E-42d) (450 mg, 1.21 mmol) in MeOH (10 mL) was added Pd/C (10%) (150 mg, 1.41 mmol) and the reaction mixture was allowed to stir under hydrogen atmosphere at room temperature. The reaction progress was monitored by TLC/LCMS analysis. After completion of the reaction (2 h), the catalyst was filtered off through a celite pad and washed with MeOH (3×15 mL). The filtrate was concentrated under reduced pressure and the residue was purified by Combi-Flash (using gradient elution of 0-70% of EtOAc in heptane) to afforded desired compound E-42e (326 mg) as a pale yellow semi solid. LCMS (ES) m/z: 342.2 [M+H]⁺.

Step-6: Synthesis of 3-(3-((N-methylsulfamoyl)amino)benzyl)-2-oxo-3,4-dihydro-2H-benzo[e][1,3]oxazin-7-yl dimethylcarbamate (E-42): Example E-42 (34 mg) was synthesized by following procedure as described for the synthesis of E-17 (step-4) using 3-(3-aminophenyl)methyl]-2-oxo-3,4-dihydro-2H-1,3-benzoxazin-7-yl N,N-dimethylcarbamate (E-42e) (150 mg, 439 μmol) as the starting material. The final compound was purified by reverse phase preparative HPLC purification [using Eclipse C18 (150 mm×21 mm, 7 m particle size) column 0.1% ammonia in water and acetonitrile as mobile phase with 19 mL/min flow rate]. LCMS (ES) m/z: 435.3 [M+H]⁺. ¹H NMR (400 MHz, DMSO-d₆): δ 9.65 (s, 1H); 7.30-7.21 (m, 3H); 7.13 (t, J=1.6 Hz, 2H); 6.98 (d, J=7.6 Hz, 1H); 6.91 (d, J=2.4 Hz, 2H); 4.58 (s, 2H); 4.42 (s, 2H); 3.03 (s, 3H); 2.96 (s, 3H); 2.38 (d, J=5.2 Hz, 3H).

### Example 43: Preparation of 7-(cyclopropylmethoxy)-3-({2-fluoro-3-(methylsulfamoyl)amino]phenyl}methyl)-3,4-dihydro-2H-1,3-benzoxazin-2-one (E-43; Compound 106)

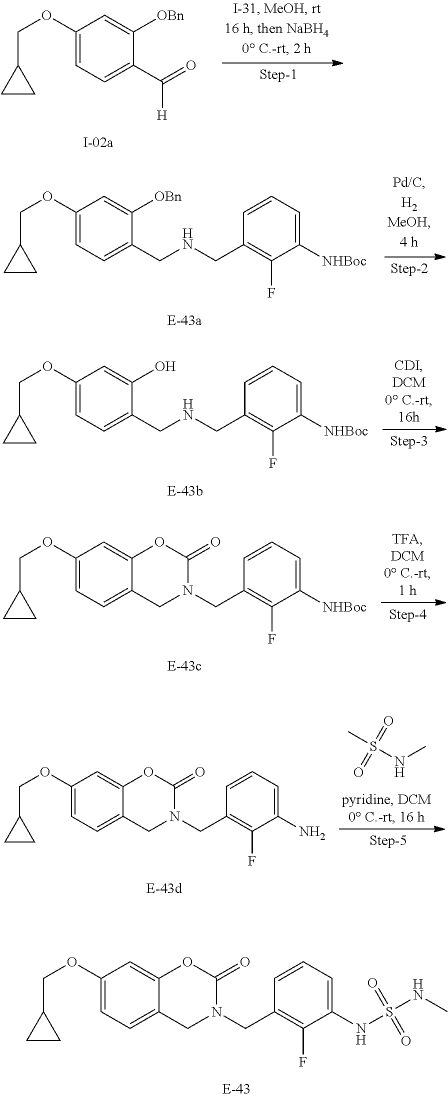

Step-1: Synthesis of tert-butyl (3-(((2-(benzyloxy)-4-(cyclopropylmethoxy)benzyl)amino)methyl)-2-fluorophenyl)carbamate (E-43a): Compound E-39a (0.680 g) was synthesized by following procedure as described for the synthesis of E-01 (step-1) using 2-(benzyloxy)-4-(cyclopropylmethoxy)benzaldehyde (I-02a) (580 mg, 2.05 mmol) and tert-butyl N-[3-(aminomethyl)-2-fluorophenyl]carbamate (I-31) (592 mg, 2.47 mmol) as the starting materials. LCMS (ES) m/z: 507.2 [M+H]⁺.

Step-2: Synthesis of tert-butyl (3-(((4-(cyclopropylmethoxy)-2-hydroxybenzyl)amino)methyl)-2-fluorophenyl)carbamate (E-43b): To a stirred solution of tert-butyl N-{3-[({[2-(benzyloxy)-4-(cyclopropylmethoxy)phenyl]methyl}amino)methyl]-2-fluorophenyl}carbamate (E-43a) (680 mg, 1.34 mmol) in MeOH (10 mL) was added Pd/C (10%) (72 mg, 671 Flmol) and the reaction mixture was allowed to stir at room temperature under hydrogen atmosphere. The reaction progress was monitored by TLC/LCMS. After complete consumption of starting material (4 h), the catalyst was filtered off through a celite pad and washed with MeOH (3×15 mL). The filtrate was concentrated under reduced pressure and the residue was purified by Combi-Flash (using gradient elution of 0-70% of EtOAc in heptane) to afforded desired compound E-43b (600 mg) as a pale yellow semi solid. LCMS (ES) m/z: 417.2 [M+H]⁺.

Step-3: Synthesis of tert-butyl (3-((7-(cyclopropylmethoxy)-2-oxo-2H-benzo[e][1,3]oxazin-3(4H)-yl)methyl)-2-fluorophenyl)carbamate (E-43c): Compound E-43c (0.600 g) was synthesized by following procedure as described for the synthesis of E-17 (step-3) using tert-butyl N-{3-[({[4-(cyclopropylmethoxy)-2-hydroxyphenyl]methyl}amino)methyl]-2-fluorophenyl}carbamate (E-43b) (680 mg, 1.63 mmol) as the starting material. LCMS (ES) m/z: 387.3 [(M-^{t}Bu)+H]⁺.

Step-4: Synthesis of 3-(3-amino-2-fluorobenzyl)-7-(cyclopropylmethoxy)-3,4-dihydro-2H-benzo[e][1,3]oxazin-2-one (E-43d): To a stirred solution of tert-butyl N-(3-{[7-(cyclopropylmethoxy)-2-oxo-3,4-dihydro-2H-1,3-benzoxazin-3-yl]methyl}-2-fluorophenyl) carbamate (E-43c) (0.6 g, 1.36 mmol) in DCM (5 mL) was added TFA (7 mL) at 0° C. and the reaction mixture was allowed to warm to room temperature over 1 h. The progress of the reaction was monitored by TLC/LCMS. After complete consumption of starting material (1 h), volatiles were removed under reduced pressure and saturated NaHCO₃ solution (15 mL) was added to the residue. Extraction was carried out using DCM (3×15 mL); the combined organic extracts were dried over anhydrous Na₂SO₄, filtered and concentrated under reduced pressure. The residue was purified by Combi-Flash (using gradient elution of 0-50% of EtOAc in heptane) afforded the desired compound E-43d (500 mg) as an off-white solid. LCMS (ES) m/z: 343.2 [M+H]⁺.

Step-5: Synthesis of 7-(cyclopropylmethoxy)-3-({2-fluoro-3-(methylsulfamoyl)amino]phenyl}methyl)-3,4-dihydro-2H-1,3-benzoxazin-2-one (E-43): Example E-43 (0.3 g) was synthesized by following procedure as described for the synthesis of E-17 (step-4) using 3-[(3-amino-2-fluorophenyl)methyl]-7-(cyclopropylmethoxy)-3,4-dihydro-2H-1,3-benzoxazin-2-one (E-43d) (0.5 g, 1.46 mmol) as the starting material. The final compound was purified by reverse phase preparative HPLC purification [using X-Bridge C-18 (250 mm×4.6 mm, 5 m particle size) column, 0.1% ammonia in water and acetonitrile as mobile phase with 1.0 mL/min flow rate]. LCMS (ES) m/z: 436.2 [M+H]⁺. ¹H NMR (400 MHz, DMSO d₆): δ 9.41 (s, 1H); 7.42-7.36 (m, 1H); 7.20 (q, J=4.8 Hz, 1H); 7.15-7.09 (m, 3H); 6.70 (dd, J, =2.4 Hz, J₂=8.4 Hz, 1H); 6.62 (d, J=2.4 Hz, 1H); 4.65 (s, 2H); 4.40 (s, 2H); 3.80 (d, J=7.2 Hz, 2H); 2.50 (d, J=5.2 Hz, 3H); 1.23-1.15 (m, 1H); 0.58-0.52 (m, 2H); 0.32-0.28 (m, 2H).

### Example 44: Preparation of 3-{[2-fluoro-3-(methylaminosulfonylamino)phenyl]methyl}-7-(3-oxetanyloxy)-3,4-dihydro-2H-1,3-benzoxazin-2-one (E-44; Compound 98)

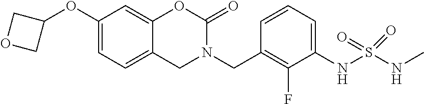 [SMILES: CNS(=O)(=O)Nc1cccc(CN2Cc3ccc(OC4COC4)cc3OC2=O)c1F]

E-44 was synthesized by following similar synthesis sequence and experimental procedures as described for the synthesis of E-43, using key intermediates I-02b and I-31. LCMS (ES) m/z: 438.3 [M+H]⁺. ¹H NMR (400 MHz, DMSO-d₆): δ 9.41 (s, 1H); 7.40-7.35 (m, 1H); 7.22 (d, J=5.2 Hz, 1H); 7.18-7.08 (m, 3H); 6.60 (dd, J₁=2.4 Hz, J₂=8.4 Hz, 1H); 6.50 (d, J=2.4 Hz, 1H); 5.29 (q, J=5.6 Hz, 1H); 4.92 (t, J=7.2 Hz, 2H); 4.65 (s, 2H); 4.53-4.48 (m, 2H); 4.40 (s, 2H); 2.50 (d, J=5.2 Hz, 3H).

### Example 45: Preparation of 3-{[2-fluoro-3-(methylaminosulfonylamino)phenyl]methyl}-7-(2-oxo-1-piperidyl)-3,4-dihydro-2H-1,3-benzoxazin-2-one (E-45; Compound 138)

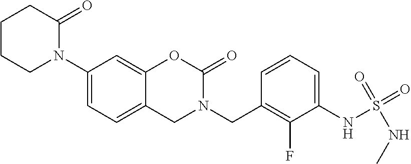 [SMILES: CNS(=O)(=O)Nc1cccc(CN2Cc3ccc(N4CCCCC4=O)cc3OC2=O)c1F]

E-45 was synthesized by following similar synthesis sequence and experimental procedures as described for the synthesis of E-43, using key intermediates I-12 and I-31. (note: step-2 led to over-saturation of pyridone ring). LCMS (ES) m/z: 463.2 |M+H|⁺. ¹H NMR (400 MHz, DMSO-d₆): δ 9.44 (s, 1H); 7.42-7.37 (m, 1H); 7.27-7.21 (m, 2H); 7.18-7.12 (m, 2H); 7.06 (dd, J₁=1.6 Hz, J₂=8.0 Hz, 1H); 7.02 (d, J=2.0 Hz, 1H); 4.68 (s, 2H); 4.48 (s, 2H); 3.59 (t, J=4.0 Hz, 2H); 2.50 (d, J=5.2 Hz, 3H); 2.39 (t, J=8.0 Hz, 2H); 1.90-1.82 (m, 4H).

### Example 46: Preparation of 3-{[2-fluoro-3-(methylaminosulfonylamino)phenyl]methyl}-7-(1-methyl-3-pyrazolyloxy)-3,4-dihydro-2H-1,3-benzoxazin-2-one (E-46; Compound 16)

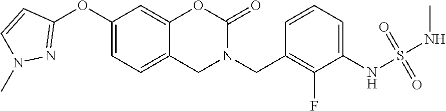 [SMILES: CNS(=O)(=O)Nc1cccc(CN2Cc3ccc(Oc4ccn(C)n4)cc3OC2=O)c1F]

E-46 was synthesized by following similar synthesis sequence and experimental procedures as described for the synthesis of E-43, using key intermediates I-44 and I-31. LCMS (ES) m/z: 462.3 [M+H]⁺. ¹H NMR (400 MHz, DMSO-d₆): δ 7.66 (d, J=2.0 Hz, 1H); 7.40-7.36 (m, 1H); 7.20 (d, J=8.8 Hz, 1H); 7.14-7.06 (m, 2H); 6.82 (dd, J₁=2.4 Hz, J₂=8.4 Hz, 1H); 6.70 (d, J=2.4 Hz, 1H); 5.86 (d, J=2.0 Hz, 1H); 4.65 (s, 2H), 4.44 (s, 2H); 3.74 (s, 3H); 2.53 (d, J=4.8 Hz, 3H).

### Example 47: Preparation of 6-chloro-3-(2-fluoro-3-((N-methylsulfamoyl)amino)benzyl)-2-oxo-3,4-dihydro-2H-benzo[e][1,3]oxazin-7-yl dimethylcarbamate (E-47; Compound 149)

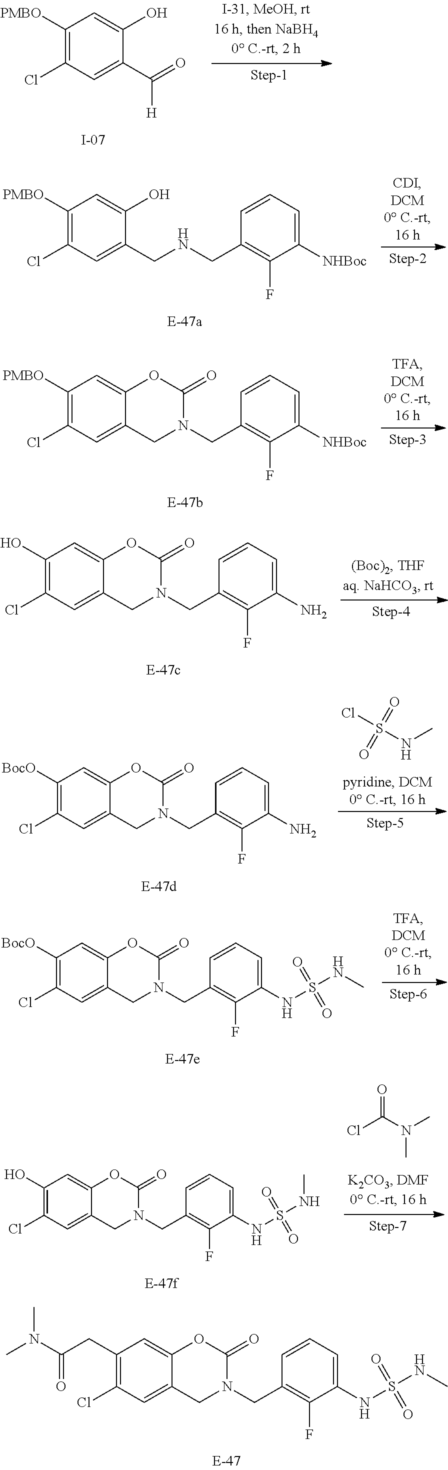

Step-1: Synthesis of tert-butyl (3-(((5-chloro-2-hydroxy-4-((4-methoxybenzyl)oxy)benzyl)amino)methyl)-2-fluorophenyl)carbamate (E-47a): Compound E-47a (2.6 g) was synthesized by following procedure as described for the synthesis of E-01 (step-1) using 5-chloro-2-hydroxy-4-[(4-methoxyphenyl)methoxy]benzaldehyde (I-07) (1.5 g, 5.12 mmol) and tert-butyl N-[3-(aminomethyl)-2-fluorophenyl]carbamate (1-31) (1.23 g, 5.12 mmol) as the starting materials. LCMS (ES) m/z: 517.2 [M+H]⁺.

Step-2: Synthesis of tert-butyl (3-((6-chloro-7-((4-methoxybenzyl)oxy)-2-oxo-2H-benzo[e][1,3]oxazin-3(4H)-yl)methyl)-2-fluorophenyl)carbamate (E-47b): Compound E-47b (2.2 g) was synthesized by following procedure as described for the synthesis of E-01 (step-2) using tert-butyl N-(3-{[({5-chloro-2-hydroxy-4-[(4-methoxyphenyl)methoxy]phenyl}methyl)amino]methyl}-2-fluorophenyl)carbamate (E-47a) (2.6 g, 5.03 mmol) as the starting material. ¹H NMR (400 MHz, DMSO d₆): δ 8.97 (s, 1H); 7.51 (d, J=7.2 Hz, 1H); 7.35 (t, J=8.4 Hz, 3H); 7.15-7.04 (m, 2H); 6.95 (t, J=13.6 Hz, 3H); 5.16 (s, 2H); 4.79 (s, 2H); 4.61 (s, 2H); 3.73 (s, 3H); 1.42 (s, 9H).

Step-3: Synthesis of 3-(3-amino-2-fluorobenzyl)-6-chloro-7-hydroxy-3,4-dihydro-2H-benzo[e][1,3]oxazin-2-one (E-47c): To a stirred solution of tert-butyl N-[3-({6-chloro-7-(4-methoxyphenyl)methoxy]-2-oxo-3,4-dihydro-2H-1,3-benzoxazin-3-yl}methyl)-2-fluorophenyl]carbamate (E-47b) (1.3 g, 2.39 mmol) in DCM (4 mL) was added TFA (1.84 mL, 23.9 mmol) at 0° C. and the reaction mixture was allowed to warm to room temperature. The reaction progress was monitored by TLC/LCMS. After complete consumption of starting material (1 h), volatiles were removed under reduced pressure and the residue was washed with diethyl ether (3×10 mL) to afford desired compound E-47c (0.9 g). LCMS (ES) m/z: 323.1 [M+H]⁺.

Step-4: Synthesis of 3-(3-amino-2-fluorobenzyl)-6-chloro-2-oxo-3,4-dihydro-2H-benzo[e][1,3]oxazin-7-yl tert-butyl carbonate (E-47d): To a stirred solution of 3-[(3-amino-2-fluorophenyl)methyl]-6-chloro-7-hydroxy-3,4-dihydro-2H-1,3-benzoxazin-2-one (E-47c) (0.5 g, 1.55 mmol) in THE (5 mL) and water (5 mL) was added NaHCO₃ (1.3 g, 15.5 mmol) at 0° C. and the reaction mixture was allowed to stir for 15 min. To this was then (Boc)₂O (0.35 mL, 1.55 mmol) and reaction mixture was allowed to stir at room temperature for 16 h. Water (30 mL) was then added to it and extraction was carried out using EtOAc (2×30 mL). The combined organic extracts were washed with saturated NaHCO₃ solution (2×20 mL), brine solution (2×20 mL), dried over anhydrous Na₂SO₄, filtered and concentrated under reduced pressure. The residue was purified by Combi-Flash (using gradient elution of 0-60% of EtOAc in heptane) to afford desired compound E-47d (0.35 g) as an off white solid. LCMS (ES) n/z: 423.1 [M+H]⁺.

Step-5: Synthesis of tert-butyl (6-chloro-3-(2-fluoro-3-((N-methylsulfamoyl)amino)benzyl)-2-oxo-3,4-dihydro-2H-benzo[e][1,3]oxazin-7-yl) carbonate (E-47e): Compound E-47e (0.3 g) was synthesized by following procedure as described for the synthesis of E-17 (step-4) using 3-[(3-amino-2-fluorophenyl)methyl]-6-chloro-2-oxo-3,4-dihydro-2H-1,3-benzoxazin-7-yl tert-butyl carbonate (E-47d) (0.3 g, 0.7 mmol) as the starting material. LCMS (ES) m/z: 513.9 [M−H]⁺.

Step-6: Synthesis of 6-chloro-3-({2-fluoro-3-(methylsulfamoyl)amino]phenyl}methyl)-7-hydroxy-3,4-dihydro-2H-1,3-benzoxazin-2-one (E-47f): To a stirred solution of tert-butyl 6-chloro-3-({2-fluoro-3-(methylsulfamoyl)amino]phenyl}methyl)-2-oxo-3,4-dihydro-2H-1,3-benzoxazin-7-yl carbonate (E-47e) (0.25 g, 0.48 mmol) in DCM (2 mL) was added TFA (0.5 mL) at 0° C. and the reaction mixture was allowed to stir at room temperature. The reaction progress was monitored by TLC/LCMS. After complete consumption of starting material (1 h), volatiles were removed under reduced pressure and the residue was washed with diethyl ether (3×10 mL) to afford the desired compound E-47f (0.180 g). LCMS (ES) m/z: 416.1 [M+H]⁺.

Step-7: Synthesis of 6-chloro-3-(2-fluoro-3-((N-methylsulfamoyl)amino)benzyl)-2-oxo-3,4-dihydro-2H-benzo[e][1,3]oxazin-7-yl dimethylcarbamate (E-47): Example E-47 (0.11 g) was synthesized by following procedure as described for the synthesis of E-01 (step-4) using 6-chloro-3-({2-fluoro-3-[(methylsulfamoyl)amino]phenyl}methyl)-7-hydroxy-3,4-dihydro-2H-1,3-benzoxazin-2-one (E-47f) (0.2 g, 0.48 mmol) as the starting material. The final compound was purified by reverse phase preparative HPLC purification [using ODS C18 (250 mm×20 mm, 5 m particle size) column, 0.1% ammonia in water and acetonitrile as mobile phase with 19 mL/min flow rate]. LCMS (ES) m/z: 487.1 [M+H]⁺. ¹H NMR (400 MHz, DMSO d₆): δ 9.45 (s, 1H); 7.49 (s, 1H); 7.42-7.37 (m, 1H); 7.22-7.13 (m, 4H); 4.66 (s, 2H); 4.48 (s, 2H); 3.07 (s, 3H); 2.92 (s, 3H); 2.50 (d, J=5.2 Hz, 3H).

### Example 48: Preparation of 6-chloro-3-(2-fluoro-3-((N-methylsulfamoyl)amino)benzyl)-2-oxo-3,4-dihydro-2H-benzo[e][1,3]oxazin-7-yl isobutyrate (E-48; Compound 129)

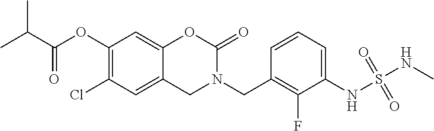 [SMILES: CNS(=O)(=O)Nc1cccc(CN2Cc3cc(Cl)c(OC(=O)C(C)C)cc3OC2=O)c1F]

E-48 was synthesized by following similar synthesis sequence and experimental procedures as described for the synthesis of E-47, using key intermediate E-47f and isobutyryl chloride as the starting materials. LCMS (ES) m/z: 486.3 [M+H]⁺. ¹H NMR (400 MHz, DMSO-d₆): δ 9.44 (s, 1H); 7.53 (s, 1H); 7.42-7.37 (m, 1H); 7.25-7.13 (m, 4H); 4.67 (s, 2H); 4.49 (s, 2H); 2.88 (q, J=7.2 Hz, 1H); 2.53 (d, J=5.2 Hz, 3H); 1.26 (d, J=7.2 Hz, 6H).

### Example 49: Preparation of 6-chloro-3-(2-fluoro-3-((N-methylsulfamoyl)amino)benzyl)-2-oxo-3,4-dihydro-2H-benzo[e][1,3]oxazin-7-yl cyclopropanecarboxylate (E-49; Compound 130)

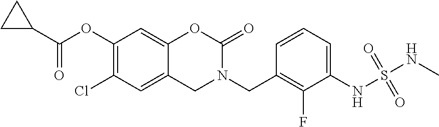 [SMILES: CNS(=O)(=O)Nc1cccc(CN2Cc3cc(Cl)c(OC(=O)C4CC4)cc3OC2=O)c1F]

E-49 was synthesized by following synthesis sequence and procedures as described for the synthesis of E-47, using key intermediate E-47f and cyclopropylcarbonyl chloride as the starting materials. LCMS (ES) m/z: 482.2 [M−H]⁺. ¹H NMR (400 MHz, DMSO-d₆): δ 9.44 (s, 1H); 7.53 (s, 1H); 7.42-7.37 (m, 1H); 7.22-7.14 (m, 4H); 4.66 (s, 2H); 4.48 (s, 2H); 2.51 (d, J=5.2 Hz, 3H); 1.97-1.93 (m, 1H); 1.14-1.06 (m, 4H).

### Example 50: Preparation of 6-chloro-3-({2-fluoro-3-[(methylsulfamoyl)amino]phenyl}methyl)-7-(pyrimidin-2-yloxy)-3,4-dihydro-2H-1,3-benzoxazin-2-one (E-50; Compound 148)

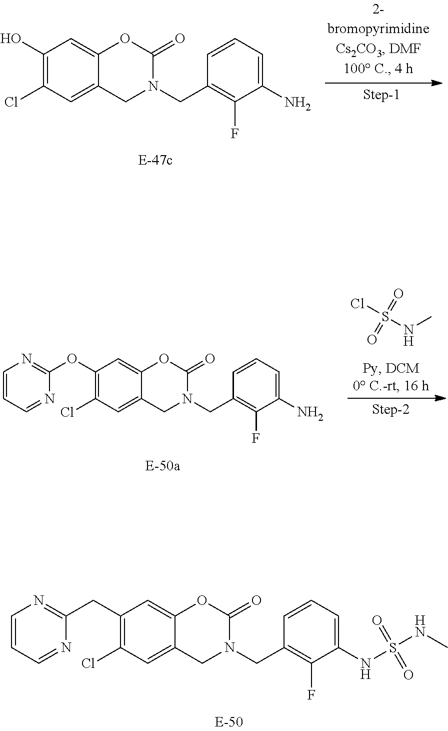

Step-1: Synthesis of 3-(3-amino-2-fluorobenzyl)-6-chloro-7-(pyrimidin-2-yloxy)-3,4-dihydro-2H-benzo[e][1,3]oxazin-2-one (E-50a): To a stirred solution of 3-[(3-amino-2-fluorophenyl)methyl]-6-chloro-7-hydroxy-3,4-dihydro-2H-1,3-benzoxazin-2-one (E-47c) (0.25 g, 0.77 mmol) in DMF (4 mL) was added 2-bromopyrimidine (0.24 g, 1.55 mmol) and Cs₂CO₃ (0.3 g, 0.93 mmol). The reaction mixture was allowed to stir at 100° C., while monitoring reaction progress by TLC/LCMS. After complete consumption of stating material (4 h), the reaction mixture was cooled temperature and quenched with water (30 mL). Extraction was carried out using EtOAc (2×20 mL); the combined organic extracts were washed with brine solution (2×20 mL), dried over anhydrous Na₂SO₄, filtered and concentrated under reduced pressure. The residue was purified by Combi-Flash (using gradient elution of 0-8% of MeOH in DCM) to afford the desired compound E-50a (0.2 g) as an off white solid. LCMS (ES) m/z: 401.0 [M+H]⁺.

Step-2: Synthesis of 6-chloro-3-({2-fluoro-3-(methylsulfamoyl)amino]phenyl}methyl)-7-(pyrimidin-2-yloxy)-3,4-dihydro-2H-1,3-benzoxazin-2-one (E-50): Example E-50 (0.074 g) was synthesized by following procedure as described for the synthesis of E-17 (step-4) using 3-[(3-amino-2-fluorophenyl)methyl]-6-chloro-7-(pyrimidin-2-yloxy)-3,4-dihydro-2H-1,3-benzoxazin-2-one (E-50a) (0.3 g, 0.74 mmol) as the starting material. The final compound was purified by reverse phase preparative HPLC purification [using X-Bridge C18 (19 mm×250 mm, 5 m particle size) column, 0.1% ammonia in water and acetonitrile as mobile phase with 18 mL/min flow rate]. LCMS (ES) m/z: 494.2 [M+H]⁺. ¹H NMR (400 MHz, DMSO d₆): δ 9.45 (s, 1H); 8.67 (d, J=4.8 Hz, 2H); 7.55 (s, 1H); 7.42-7.38 (m, 1H); 7.32 (t, J=4.8 Hz, 1H); 7.27 (s, 1H); 7.22-7.15 (m, 3H); 4.68 (s, 2H); 4.52 (s, 2H); 2.53 (d, J=4.8 Hz, 3H).

### Example 51: Preparation of 3-(2-fluoro-3-((N-methylsulfamoyl)amino)benzyl)-2-oxo-3,4-dihydro-2H-benzo[e][1,3]oxazin-7-yl 3,3-difluoroazetidine-1-carboxylate (E-51; Compound 146)

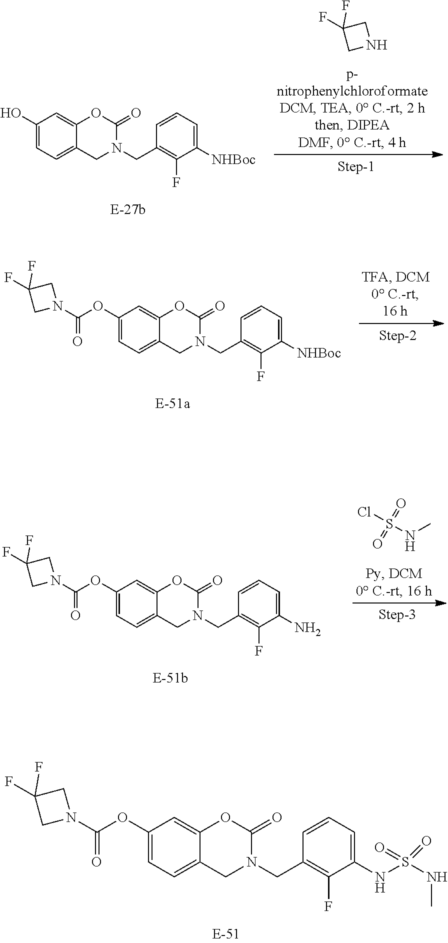

Step-1: Synthesis of 3-(3-((tert-butoxycarbonyl)amino)-2-fluorobenzyl)-2-oxo-3,4-dihydro-2H-benzo[e][1,3]oxazin-7-yl 3,3-difluoroazetidine-1-carboxylate (E-51a): To a stirred solution of tert-butyl N-{2-fluoro-3-[(7-hydroxy-2-oxo-3,4-dihydro-2H-1,3-benzoxazin-3-yl)methyl]phenyl}carbamate (E-27b) (0.3 g, 772 Pmol) in DCM (10 mL) was added TEA (434 μL, 3.09 mmol) and 4-nitrophenyl chloroformate (202 mg, 1 mmol) at 0° C. and the reaction mixture was stirred at room temperature for 2 h. Volatiles were then evaporated under reduced pressure and the residue was dissolved in DMF (3 mL). To this was then added DIPEA (210 mg, 1.63 mmol) and 3,3-difluoroazetidine hydrochloride (140 mg, 1.08 mmol) at 0° C. The reaction mixture was then stirred at room temperature for 4 h. After complete consumption of starting material, water (20 mL) was added to it and extraction was carried out using EtOAc (30 mL×2). The combined organic extracts were washed with brine (30 mL×3), dried over anhydrous Na₂SO₄, filtered and concentrated under reduced pressure. The residue was purified by Combi-Flash (using gradient elution of 0-60% of EtOAc in heptane) to afford desired compound E-51a (0.2 g) as a white solid. LCMS (ES) m/z: 452.1 [(M-^{t}Bu)+H]⁺.

Step-2: Synthesis of 3-(3-amino-2-fluorobenzyl)-2-oxo-3,4-dihydro-2H-benzo[e][1,3]oxazin-7-yl 3,3-difluoroazetidine-1-carboxylate (E-51b): Compound E-51b (130 mg) was synthesized by following procedure as described for the synthesis of E-43 (step-4) using 3-[(3-{[(tert-butoxy)carbonyl]amino}-2-fluorophenyl)methyl]-2-oxo-3,4-dihydro-2H-1,3-benzoxazin-7-yl 3,3-difluoroazetidine-1-carboxylate (E-51a) (0.2 g, 394 μmol) as the starting material. LCMS (ES) m/z: 408.1 [M+H]⁺.

Step-3: Synthesis of 3-(2-fluoro-3-((N-methylsulfamoyl)amino)benzyl)-2-oxo-3,4-dihydro-2H-benzo[e][1,3]oxazin-7-yl 3,3-difluoroazetidine-1-carboxylate (E-51): Example E-51 (0.074 g) was synthesized by following procedure as described for the synthesis of E-17 (step-4) using 3-[(3-amino-2-fluorophenyl)methyl]-2-oxo-3,4-dihydro-2H-1,3-benzoxazin-7-yl 3,3-difluoroazetidine-1-carboxylate (E-51b) (120 mg, 295 μmol) as the starting material. The final compound was purified by reverse phase preparative HPLC purification [using Kinetex EVO C18 (100 mm×2.1 mm, 1.7 μm particle size) column, 0.1% formic acid in water and acetonitrile as mobile phase with 0.5 mL/min flow rate]. LCMS (ES) m/z: 499.2 [M−H]⁺. ¹H NMR (400 MHz, DMSO d₆): δ 9.43 (s, 1H); 7.44-7.36 (m, 1H); 7.27 (d, J=8.8 Hz, 1H); 7.24-7.10 (m, 3H); 7.00-6.92 (m, 2H); 4.67 (s, 2H); 4.64-4.40 (m, 4H); 4.49 (s, 2H); 2.50 (d, J=4.8 Hz, 3H).

### Example 52: Preparation of 3-(2-fluoro-3-((N-methylsulfamoyl)amino)benzyl)-N-methyl-2-oxo-3,4-dihydro-2H-benzo[e][1,3]oxazine-7-carboxamide (E-52; Compound 114)

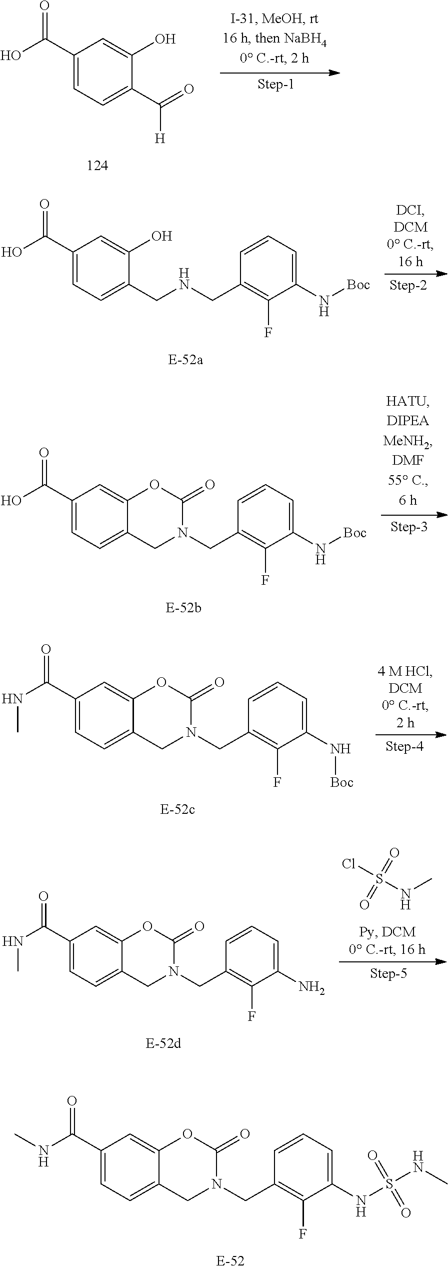

Step-1: Synthesis of 4-({[(3-{[(tert-butoxy)carbonyl]amino}-2-fluorophenyl)methyl]amino}methyl)-3-hydroxybenzoic acid (E-52a): Compound E-52a (1.3 g) was synthesized by following procedure as described for the synthesis of E-01 (step-1) using 4-formyl-3-hydroxybenzoic acid (1.0 g, 6.02 mmol) and tert-butyl N-13-(aminomethyl)-2-fluorophenyl]carbamate (I-31) (1.45 g, 6.02 mmol) as the starting materials. LCMS (ES) m/z: 388.94 [M−H]⁺.

Step-2: Synthesis of 3-[(3-{[(tert-butoxy)carbonyl]amino}-2-fluorophenyl)methyl]-2-oxo-3,4-dihydro-2H-1,3-benzoxazine-7-carboxylic acid (E-52b): Compound E-52b (1.1 g) was synthesized by following procedure as described for the synthesis of E-01 (step-2) using 4-(11(3-{I(tert-butoxy)carbonyl|amino}-2-fluorophenyl)methyl]amino}methyl)-3-hydroxybenzoic acid (E-52a) (1.5 g, 3.84 mmol) as the starting material. LCMS (ES) m/z: 415.19 [M+H]⁺.

Step-3: Synthesis of tert-butyl N-(2-fluoro-3-{[7-(methylcarbamoyl)-2-oxo-3,4-dihydro-2H-1,3-benzoxazin-3-yl]methyl}phenyl)carbamate (E-52c): To a stirred solution of 3-[(3-{[(tert-butoxy)carbonyl]amino}-2-fluorophenyl)methyl]-2-oxo-3,4-dihydro-2H-1,3-benzoxazine-7-carboxylic acid (E-52b) (1.1 g, 2.64 mmol) in DMF (15 mL) was added DIPEA (1.61 mL, 9.25 mmol), HATU (1.24 g, 5.28 mmol) and MeNH₂ (0.164 g, 5.28 mmol) at room temperature. The reaction mixture was then stirred at 55° C., while monitoring reaction progress by TLC. After completion of the reaction (6 h), it was quenched with water (15 mL) and extraction was carried out with EtOAc (3×30 mL). The combined organic extracts were washed with brine solution (2×30 mL), dried over anhydrous Na₂SO₄, filtered and concentrated under reduced pressure. The residue was purified by Combi-Flash (using gradient elution of 0-50% of EtOAc in heptane) to afford the desired compound E-52c (0.43 g) as an off white solid. LCMS (ES) m/z: 428.23 [M−H]⁺.

Step-4: Synthesis of 3-[(3-amino-2-fluorophenyl)methyl]-N-methyl-2-oxo-3,4-dihydro-2H-1,3-benzoxazine-7-carboxamide (E-52d): Compound E-52d (0.38 g) was synthesized by following procedure as described for the synthesis of E-17 (step-3) using tert-butyl N-(2-fluoro-3-{[7-(methylcarbamoyl)-2-oxo-3,4-dihydro-2H-1,3-benzoxazin-3-yl]methyl}phenyl)carbamate (E-52c) (0.43 g, 1 mmol) as the starting material. LCMS (ES) m/z: 330.22 [M+H]⁺.

Step-5: Synthesis of 3-({2-fluoro-3-[(methylsulfamoyl)amino]phenyl}methyl)-N-methyl-2-oxo-3,4-dihydro-2H-1,3-benzoxazine-7-carboxamide (E-52): Example E-52 (0.015 g) was synthesized by following procedure as described for the synthesis of E-17 (step-4) using 3-[(3-amino-2-fluorophenyl)methyl]-N-methyl-2-oxo-3,4-dihydro-2H-1,3-benzoxazine-7-carboxamide (E-52d) (0.38 g, 1.15 mmol) as the starting material. The final compound was purified by reverse phase preparative HPLC purification [using Sun Fire C18 (19 mm×250 mm, 5 m particle size) column, 0.1% trifluoro acetic acid and acetonitrile as mobile phase with 0.5 mL/min flow rate]. LCMS (ES) m/z: 423.40 [M+H]⁺.

¹H NMR (400 MHz, DMSO-d₆): δ 9.45 (s, 1H); 8.49 (q, J=4.4 Hz, 1H); 7.59 (dd, J₁=1.6 Hz, J₂=8.0 Hz, 1H); 7.47 (d, J=1.6 Hz, 1H); 7.41-7.36 (m, 1H); 7.32 (d, J=8.0 Hz, 1H); 7.27-7.20 (m, 1H); 7.17-7.12 (m, 2H); 4.67 (s, 2H); 4.53 (s, 2H); 2.78 (d, J=4.4 Hz, 3H); 2.51 (d, J=4.4 Hz, 3H).

### Example 53: Preparation of 7-(cyclopropylmethoxy)-3-({2-fluoro-3-(methylsulfamoyl)amino]phenyl}methyl)-3,4-dihydro-2H-1,3-benzoxazin-2-one (E-53; Compound 132)

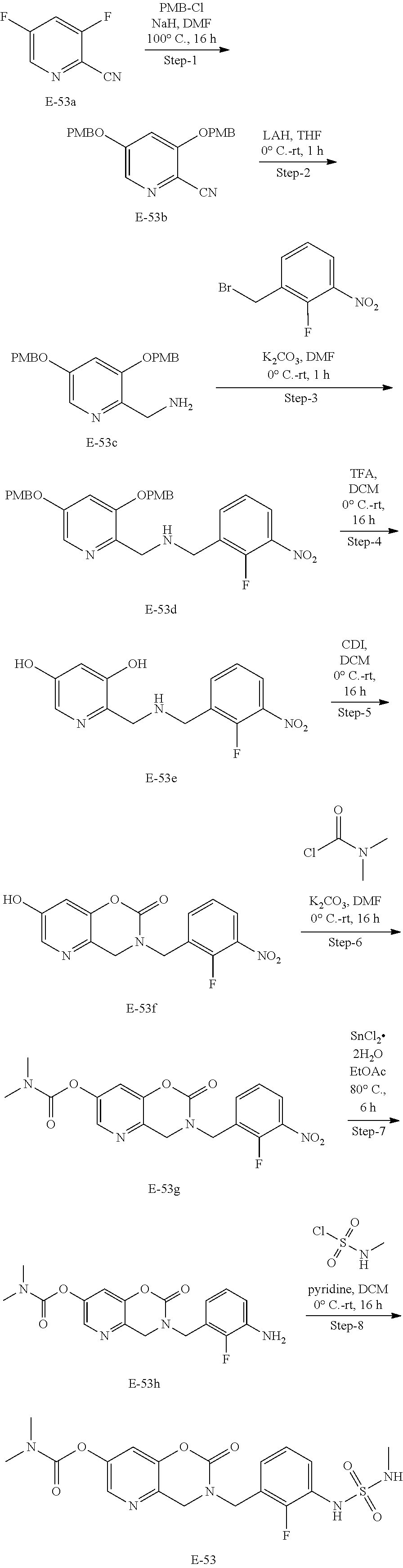

Step-1: Synthesis of 3,5-bis[(4-methoxyphenyl)methoxy]pyridine-2-carbonitrile (E-53b): To a stirred solution of (4-methoxyphenyl)methanol (9.86 g, 71.4 mmol) in DMF (50 mL) was added sodium hydride (60% dispersion in mineral oil) (1.71 g, 71.4 mmol) at 0° C. and the reaction mixture was warmed to room temperature over 30 min. To this was then added 3,5-difluoropyridine-2-carbonitrile (E-53a) (5.0 g, 35.7 mmol) and the reaction mixture was stirred at 100° C. for 16 h. After completion of the reaction, the reaction mixture was cooled to room temperature and quenched with ice cold water (15 mL). The precipitate obtained was collected by filtration and washed with water to afford the desired compound E-53b (10.0 g) as a white solid. ¹H NMR (400 MHz, DMSO-d₆): δ 8.01 (d, J=2.4 Hz, 1H); 7.54 (d, J=2.4 Hz, 1H); 7.43 (dd, J=8.4, 1.6 Hz, 4H); 7.00-6.98 (m, 4H); 5.28 (s, 2H); 5.23 (s, 2H); 3.78 (s, 6H).

Step-2: Synthesis of 1-{3,5-bis[(4-methoxyphenyl)methoxy]pyridine-2-yl}methanamine (E-53c): To a stirred solution of 3,5-bis[(4-methoxyphenyl)methoxy]pyridine-2-carbonitrile (E-53b) (10.0 g, 26.6 mmol) in anhydrous THE (100 mL) was added 2M lithium aluminium hydride (in THF) (2.02 g, 53.1 mmol) at 0° C. The reaction mixture was then warmed to room temperature and stirred for 1 h. After completion of the reaction (1 h), it was quenched with slow addition of ice cold water (15 mL). Extraction was carried out using EtOAc (3×40 mL); the combined organic extracts were washed with brine solution, dried over Na₂SO₄ and concentrated under reduced pressure. The residue was purified by Combi-Flash (using gradient elution of 0-10% of MeOH in DCM) to afford the desired compound E-53c (4.8 g) as a colorless liquid. LCMS (ES) m/z: 381.46 [M+H]⁺.

Step-3: Synthesis of ({3,5-bis[(4-methoxyphenyl)methoxy]pyridine-2-yl}methyl)[(2-fluoro-3-nitrophenyl)methyl]amine (E-53d): To a stirred solution of 1-{3,5-bis[(4-methoxyphenyl)methoxy]pyridine-2-yl}methanamine (E-53c) (4.8 g, 12.6 mmol) in DMF (48 mL) was added K₂CO₃ (5.23 g, 37.9 mmol) and 1-(bromomethyl)-2-fluoro-3-nitrobenzene (3.54 g, 15.1 mmol) at 0° C. The reaction mixture was stirred at room temperature. After completion of the reaction (1 h), ice cold water (25 mL) was added to it and extraction was carried out using EtOAc (3×30 mL). The combined organic extracts were washed with brine solution (30 mL×2), dried over Na₂SO₄ and concentrated under reduced pressure. The residue was purified by Combi-Flash (using gradient elution of 0-5% of MeOH in DCM) to afford the desired compound E-53d (3.5 g) as light brown solid. LCMS (ES) m/z: 534.43 [M+H]⁺.

Step-4: Synthesis of 2-({[(2-fluoro-3-nitrophenyl)methyl]amino}methyl)pyridine-3,5-diol (E-53e): To a stirred solution of ({3,5-bis[(4-methoxyphenyl)methoxy]pyridine-2-yl}methyl)[(2-fluoro-3-nitrophenyl)methyl]amine (E-53d) (3.5 g, 6.56 mmol) in trifluoroacetic acid (2 mL) was added pentamethylbenzene (5.83 g, 39.4 mmol) at 0° C. and the reaction mixture was stirred at room temperature for 24 h. Volatiles were then removed under reduced pressure and saturated NaHCO₃ solution (30 mL) was added to the residue. Extraction was carried out using EtOAc (3×30 mL); the combined organic extracts were washed with brine solution (30 mL), dried over Na₂SO₄ and concentrated under reduced pressure. The residue was purified by Combi-Flash (using gradient elution of 0-10% of MeOH in DCM) to afford the desired compound E-53e (0.85 g) as yellow solid. LCMS (ES) m/z: 294.27 [M+H]⁺.

Step-5: Synthesis of 3-[(2-fluoro-3-nitrophenyl)methyl]-7-hydroxy-2H,3H,4H-pyrido[2,3-e][1,3]oxazin-2-one (E-53f): Compound E-53f (0.65 g) was synthesized by following procedure as described for the synthesis of E-01 (step-2) using 2-({[(2-fluoro-3-nitrophenyl)methyl]amino}methyl)pyridine-3,5-diol (E-53e) (0.85 g, 2.9 mmol) as the starting material. LCMS (ES) m/z: 318.51 [M−H]⁺.

Step-6: Synthesis of 3-[(2-fluoro-3-nitrophenyl)methyl]-2-oxo-2H,3H,4H-pyrido[2,3-e][1,3]oxazin-7-yl N,N-dimethylcarbamate (E-53g): Compound E-53g (0.290 g) was synthesized by following procedure as described for the synthesis of E-01 (step-4) 3-[(2-fluoro-3-nitrophenyl)methyl]-7-hydroxy-2H,3H,4H-pyrido[2,3-e][1,3]oxazin-2-one (E-53f) (0.35 g, 1.1 mmol) as the starting material. LCMS (ES) m/z: 391.42 [M+H]⁺.

Step-7: Synthesis of 3-[(3-amino-2-fluorophenyl)methyl]-2-oxo-2H,3H,4H-pyrido[2,3-e][1,3]oxazin-7-yl N,N-dimethylcarbamate (E-53h): Compound E-53h (0.14 g) was synthesized by following procedure as described for the synthesis of E-41 (step-3) using 3-[(2-fluoro-3-nitrophenyl)methyl]-2-oxo-2H,3H,4H-pyrido[2,3-e][1,3]oxazin-7-yl N,N-dimethylcarbamate (E-53g) (0.2 g, 0.512 mmol) as the starting material. LCMS (ES) m/z: 361.37 [M+H]⁺.

Step-8: Synthesis of 3-({2-fluoro-3-[(methylsulfamoyl)amino]phenyl}methyl)-2-oxo-2H,3H,4H-pyrido[2,3-e][1,3]oxazin-7-yl N,N-dimethylcarbamate (E-53): Example E-53 (0.027 g) was synthesized by following procedure as described for the synthesis of E-41 (step-4) using 3-[(3-amino-2-fluorophenyl)methyl]-2-oxo-2H,3H,4H-pyrido[2,3-e][1,3]oxazin-7-yl N,N-dimethylcarbamate (E-53h) (0.140 g, 0.389 mmol) as the starting material. The final compound was purified by reverse phase preparative HPLC purification [using X Bridge C18 (19 mm×250 mm, 10 μm particle size) column, using acetonitrile and 5 mM ammonium bicarbonate in water as mobile phase with 19.0 mL/min flow rate]. LCMS (ES) m/z: 454.19 [M+H]⁺. ¹H NMR (400 MHz, DMSO-d₆): (8.19 (d, J=2.0 Hz, 1H); 7.48 (d, J=2.4 Hz, 1H); 7.39 (td, J₁=1.6 Hz, J₂=7.6 Hz, 1H); 7.20-7.10 (m, 2H); 4.68 (s, 2H); 4.55 (s, 2H); 3.05 (s, 3H); 2.92 (s, 3H); 2.50 (d, J=4.4 Hz, 3H).

### Example 54: Preparation of N-(3-(2-fluoro-3-((N-methylsulfamoyl)amino)benzyl)-2-oxo-3,4-dihydro-2H-benzo[e][1,3]oxazin-7-yl)-1H-pyrazole-4-carboxamide (E-54; Compound 122)

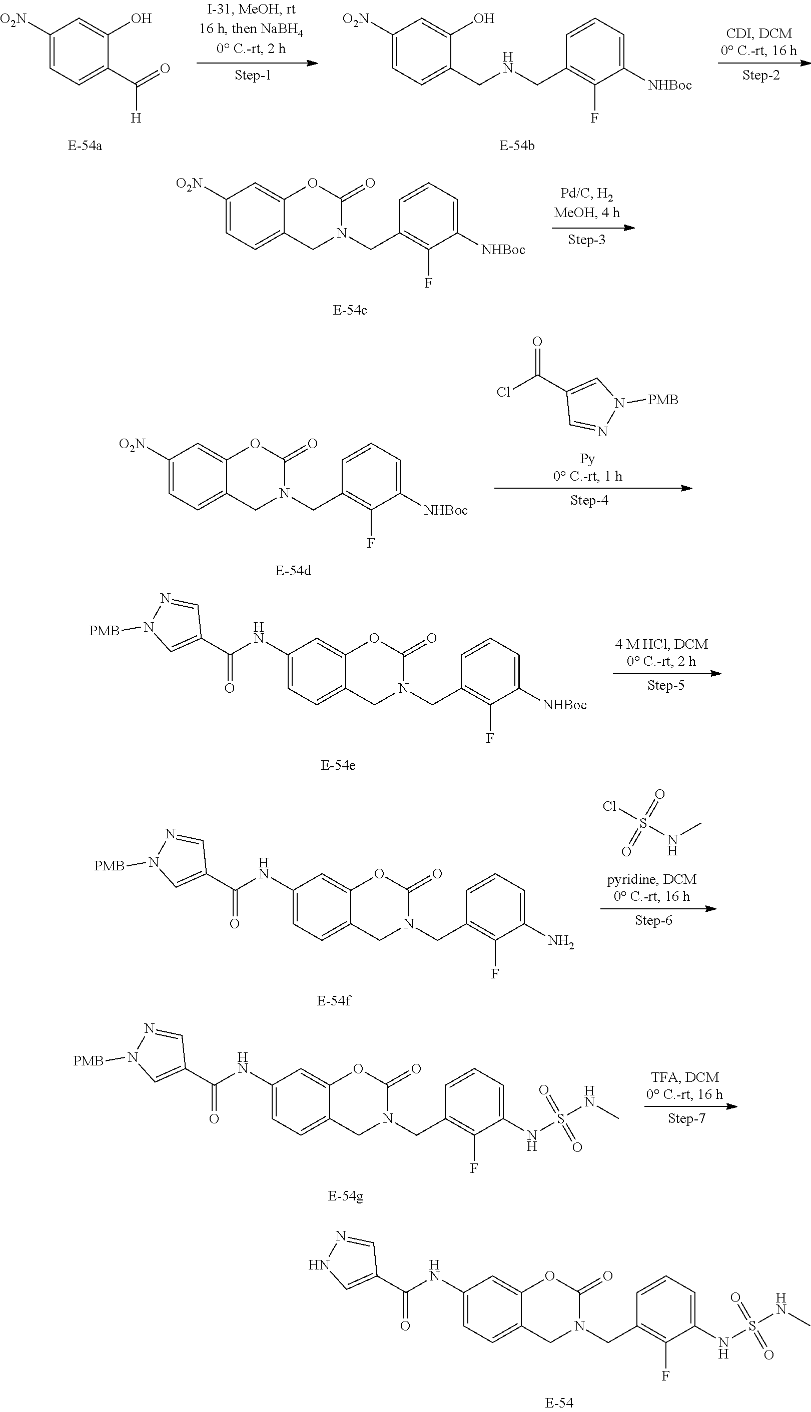

Step-1: Synthesis of tert-butyl N-[2-fluoro-3-({[(2-hydroxy-4-nitrophenyl)methyl]amino}methyl)phenyl]carbamate (E-54b): Compound E-54b (4.0 g) was synthesized by following procedure as described for the synthesis of E-01 (step-1) using 2-hydroxy-4-nitrobenzaldehyde (E-54a) (2.0 g, 12 mmol) and tert-butyl N-[3-(aminomethyl)-2-fluorophenyl]carbamate (I-31) (2.88 g, 12 mmol) as the starting materials. ¹H NMR (400 MHz, DMSO-d₆): δ 8.93 (s, 1H); 7.63 (dd, J=8.0, 2.0 Hz, 1H); 7.53-7.41 (m, 3H); 7.18-7.15 (m, 1H); 7.11-7.07 (m, 1H); 3.84 (s, 2H); 3.75 (s, 2H); 1.45 (s, 9H).

Step-2: Synthesis of tert-butyl N-{2-fluoro-3-[(7-nitro-2-oxo-3,4-dihydro-2H-1,3-benzoxazin-3-yl)methyl]phenyl}carbamate (E-54c): Compound E-49c (3.9 g) was synthesized by following procedure as described for the synthesis of E-01 (step-2) using tert-butyl N-[2-fluoro-3-({[(2-hydroxy-4-nitrophenyl)methyl]amino}methyl)phenyl]carbamate (E-54b) (4.0 g, 11 mmol) as the starting material. LCMS (ES) m/z: 416.32 [M−H]⁺.

Step-3: Synthesis of tert-butyl N-{3-[(7-amino-2-oxo-3,4-dihydro-2H-1,3-benzoxazin-3-yl)methyl]-2-fluorophenyl}carbamate (E-54d): To a stirred solution of tert-butyl N-{2-fluoro-3-[(7-nitro-2-oxo-3,4-dihydro-2H-1,3-benzoxazin-3-yl)methyl]phenyl}carbamate (E-54c) (1.5 g, 3.59 mmol) in MeOH (15 ml) was added 10% Pd/C (1.0 g) and the reaction mixture was stirred under H₂ atmosphere. After complete consumption of starting material (4 h), the catalyst was filtered off through a celite pad and washed with MeOH (5 mL×2). MeOH was then evaporated under reduced pressure and the residue was purified by Combi-Flash (using gradient elution of 0-70% of EtOAc in heptane) to afford desired compound E-54d (1.1 g) as a brown solid. LCMS (ES) m/z: 388.47 [M+H]⁺.

Step-4: Synthesis of tert-butyl N-{2-fluoro-3-[(7-{1-[(4-methoxyphenyl)methyl]-1H-pyrazole-4-amido}-2-oxo-3,4-dihydro-2H-1,3-benzoxazin-3-yl)methyl]phenyl}carbamate (E-54e): To a stirred solution of tert-butyl N-{3-[(7-amino-2-oxo-3,4-dihydro-2H-1,3-benzoxazin-3-yl)methyl]-2-fluorophenyl}carbamate (E-54d) (1.1 g, 2.84 mmol) in pyridine (3 mL) was added 1-[(4-methoxyphenyl)methyl]-1H-pyrazole-4-carbonyl chloride (1.42 g, 5.68 mmol) under nitrogen at 0° C. The reaction mixture was stirred at room temperature and the reaction progress was monitored by TLC/LCMS analysis. After completion of the reaction (1 h), the reaction mixture was quenched with water (10 mL) and extraction was carried out using EtOAc (3×30 mL). The combined organic extracts were washed with brine solution (30 mL), dried over Na₂SO₄ and concentrated under reduced pressure. The residue was purified by Combi-Flash (using gradient elution of 0-5% of MeOH in DCM) to afford desired compound E-54e (1.0 g) as an off white solid. LCMS (ES) m/z: 600.41 [M−H]⁺.

Step-5: Synthesis of N-{3-[(3-amino-2-fluorophenyl)methyl]-2-oxo-3,4-dihydro-2H-1,3-benzoxazin-7-yl}-1-[(4-methoxyphenyl)methyl]-1H-pyrazole-4-carboxamide (E-54f): Compound E-54f (0.4 g) was synthesized by following procedure as described for the synthesis of E-17 (step-3) using tert-butyl N-{2-fluoro-3-[(7-{1-[(4-methoxyphenyl)methyl]-1H-pyrazole-4-amido}-2-oxo-3,4-dihydro-2H-1,3-benzoxazin-3-yl)methyl]phenyl}carbamate (E-54e) (0.55 g, 0.914 mmol) as the starting material. LCMS (ES) m/z: 502.33 [M+H]⁺.

Step-6: Synthesis of N-[3-({2-fluoro-3-(methylsulfamoyl)amino]phenyl}methyl)-2-oxo-3,4-dihydro-2H-1,3-benzoxazin-7-yl]-1-[(4-methoxyphenyl)methyl]-1H-pyrazole-4-carboxamide (E-54g): Compound E-54g (0.4 g) was synthesized by following procedure as described for the synthesis of E-17 (step-4) using N-{3-[(3-amino-2-fluorophenyl)methyl]-2-oxo-3,4-dihydro-2H-1,3-benzoxazin-7-yl}-1-[(4-methoxyphenyl)methyl]-1H-pyrazole-4-carboxamide (E-54f) (0.46 g, 0.917 mmol) as the starting material. LCMS (ES) m/z: 595.04 [M+H]⁺.

Step-7: Synthesis of N-[3-({2-fluoro-3-(methylsulfamoyl)amino]phenyl}methyl)-2-oxo-3,4-dihydro-2H-1,3-benzoxazin-7-yl]-1H-pyrazole-4-carboxamide (E-54): To a stirred solution of N-[3-({2-fluoro-3-(methylsulfamoyl)amino]phenyl}methyl)-2-oxo-3,4-dihydro-2H-1,3-benzoxazin-7-yl]-1-[(4-methoxyphenyl)methyl]-1H-pyrazole-4-carboxamide (0.4 g, 0.673 mmol) in DCM (5 mL) was added TFA (4 mL) at 0° C. The reaction mixture was stirred at room temperature for 16 h. Water (15 mL) was then added to it and extraction was carried out with EtOAc (3×30 mL). The combined organic extracts were washed with brine solution (30 mL), dried over Na₂SO₄ and concentrated under reduced pressure. The residue was purified by Combi-Flash (using gradient elution of 0-5% of MeOH in DCM) followed by reverse phase preparative HPLC purification [using Sun Fire C18 (19 mm×250 mm, 5 μm particle size) column, 0.1% trifluoro acetic acid and acetonitrile as mobile phase] to afford desired E-54 (0.087 g) as a white solid. LCMS (ES) m/z: 475.15 [M+H]⁺. ¹H NMR (400 MHz, DMSO-d₆): δ 9.91 (s, 1H); 9.43 (s, 1H); 8.21 (br. s, 2H); 7.53 (d, J=2.0 Hz, 1H); 7.41 (dd, J=8.4, 2.0 Hz, 1H); 7.40-7.37 (m, 1H); 7.24-7.14 (m, 4H); 4.67 (s, 2H); 4.44 (s, 2H); 2.52 (d, J=4.4 Hz, 3H).

### Example 55: Preparation of 3-(2-fluoro-3-(3-methylureido)benzyl)-2-oxo-3,4-dihydro-2H-benzo[e][1,3]oxazin-7-yl dimethylcarbamate (E-55; Compound 128)

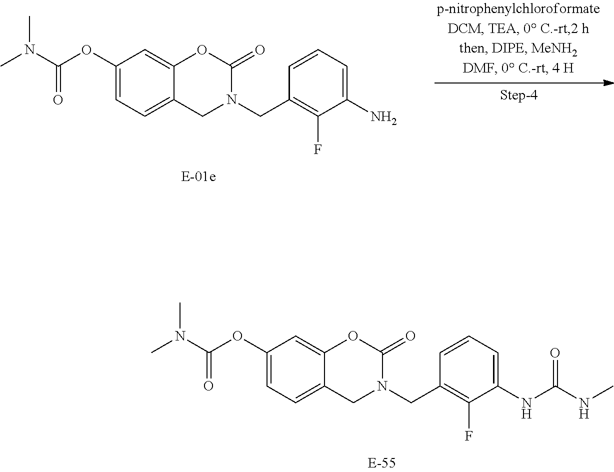

Step-1: Synthesis of 3-(2-fluoro-3-(3-methylureido)benzyl)-2-oxo-3,4-dihydro-2H-benzo[e][1,3]oxazin-7-yl dimethylcarbamate (E-55): Example 55 (0.940 g) was synthesized by following procedure as described for the synthesis of E-46 (step-1) using 3-(3-amino-2-fluorobenzyl)-2-oxo-3,4-dihydro-2H-benzo[e][1,3]oxazin-7-yl dimethylcarbamate (E-Ole) (0.2 g, 0.557 mmol) and 2M methyl amine in THF (1.4 mL, 2.78 mmol) as the starting material. The final compound was purified by Combi-Flash (using gradient elution of 0-7% of MeOH in DCM) followed by trituration using diethyl ether (3×10 mL). LCMS (ES) m/z: 417.3 [M+H]⁺. ¹H NMR (400 MHz, DMSO d₆): δ 8.34 (d, J=1.6 Hz, 1H); 8.06 (t, J=7.2 Hz, 1H); 7.23 (d, J=8.0 Hz, 1H); 7.06 (t, J=8.0 Hz, 1H); 6.93-6.87 (m, 3H); 6.44 (d, J=4.4 Hz, 1H); 4.66 (s, 2H); 4.45 (s, 2H); 3.02 (s, 3H); 2.90 (s, 3H); 2.65 (d, J=4.4 Hz, 3H).

### Example 56: Preparation of 3-(2-fluoro-3-(3-(2-methoxyethyl)ureido)benzyl)-2-oxo-3,4-dihydro-2H-benzo[e][1,3]oxazin-7-yl dimethylcarbamate (E-56; Compound 119)

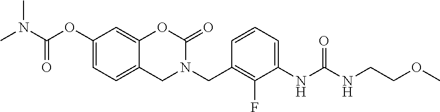

E-56 was synthesized by following similar synthesis sequence and experimental procedures as described for the synthesis of E-55, using key intermediates E-Ole and 2-methoxyethylamine as the starting materials. LCMS (ES) m/z: 461.4 [M+H]⁺. ¹H NMR (400 MHz, DMSO-d₆): δ 8.41 (d, J=2.4 Hz, 1H); 8.07 (t, J=7.2 Hz, 1H); 7.23 (d, J=8.0 Hz, 1H); 7.06 (t, J=8.0 Hz, 1H); 7.42-7.34 (m, 3H); 6.73 (t, J=5.6 Hz, 1H); 4.66 (s, 2H); 4.46 (s, 2H); 3.38 (t, J=5.2 Hz, 2H); 3.26 (s, 3H); 3.25 (t, J=5.2 Hz, 2H); 3.03 (s, 3H); 2.90 (s, 3H).

### Example 57: Preparation of 3-(3-(2,2-difluorocyclopropane-1-carboxamido)-2-fluorobenzyl)-2-oxo-3,4-dihydro-2H-benzo[e][1,3]oxazin-7-yl dimethylcarbamate (E-57; Compound 127)

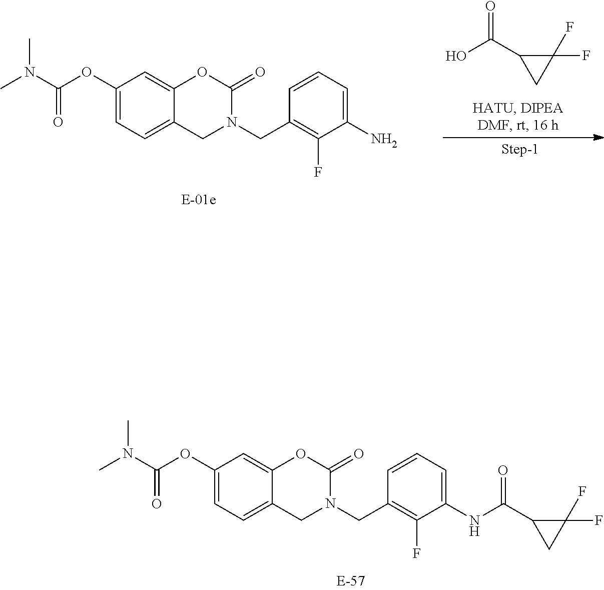 [SMILES: CN(C)C(=O)Oc1ccc2c(c1)OC(=O)N(Cc1cccc(N)c1F)C2 ; O=C(O)C1CC1(F)F ; CN(C)C(=O)Oc1ccc2c(c1)OC(=O)N(Cc1cccc(NC(=O)C3CC3(F)F)c1F)C2]

Step-1: Synthesis of 3-(3-(2,2-difluorocyclopropane-1-carboxamido)-2-fluorobenzyl)-2-oxo-3,4-dihydro-2H-benzo[e][1,3]oxazin-7-yl dimethylcarbamate (E-57): To a stirred solution of 3-[(3-amino-2-fluorophenyl)methyl]-2-oxo-3,4-dihydro-2H-1,3-benzoxazin-7-yl N,N-dimethylcarbamate (E-Ole) (0.1 g, 278 μmol) and 2,2-difluorocyclopropane-1-carboxylic acid (51 mg, 417 μmol) in DMF (3 mL) was added HATU (159 mg, 417 μmol) and DIPEA (145 μL, 835 μmol) at room temperature. The reaction mixture was stirred at the same temperature for 16 h. Water (10 mL) was then added to it and extraction was carried out using EtOAc (3×20 mL). The combined organic extracts were washed with brine solution (3×20 mL), dried over anhydrous Na₂SO₄, filtered and concentrated under reduced pressure. The residue was purified by Combi-Flash (using gradient elution of 0-6% of MeOH in DCM) followed by reverse phase preparative HPLC [using X-Select C18 (19 mm×250 mm, 5 μm particle size) column, 0.1% ammonia in water and acetonitrile as mobile phase with 19.0 mL/min flow rate] to afford desired compound E-57 (45 mg) as an off-white solid. LCMS (ES) m/z: 464.1 [M+H]⁺¹H NMR (400 MHz, DMSO d₆): δ 10.24 (s, 1H); 7.8 (t, J=5.2 Hz, 1H); 7.2 (d, J=8.0 Hz, 1H); 7.14-7.11 (m, 2H); 6.88-6.85 (m, 2H); 4.66 (s, 2H); 4.45 (s, 2H); 2.99 (s, 3H); 2.94 (t, J=3.2 Hz, 1H); 2.87 (s, 3H); 1.97-1.93 (m, 2H).

### Example 58: Preparation of 3-(2-fluoro-3-(2,2,2-trifluoroacetamido)benzyl)-2-oxo-3,4-dihydro-2H-benzo[e][1,3]oxazin-7-yl dimethyl carbamate (E-58; Compound 126)

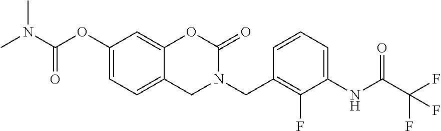 [SMILES: CN(C)C(=O)Oc1ccc2c(c1)OC(=O)N(Cc1cccc(NC(=O)C(F)(F)F)c1F)C2]

E-58 was synthesized by following synthesis sequence and procedures as described for the synthesis of E-57, using key intermediates E-Ole and trifluoroacetic acid as the starting materials. LCMS (ES) m/z: 456.3 [M+H]⁺. ¹H NMR (400 MHz, DMSO-d₆): δ 11.26 (s, 1H); 7.43 (t, J=6.4 Hz, 1H); 7.28-7.16 (m, 3H); 6.88-6.85 (m, 2H); 4.65 (s, 2H); 4.46 (s, 2H); 2.99 (s, 3H); 2.87 (s, 3H).

### Example 59: Preparation of 3-(2-fluoro-3-(2-oxo-1,2-dihydropyridin-3-yl)benzyl)-2-oxo-3,4-dihydro-2H-benzo[e][1,3]oxazin-7-yl dimethylcarbamate (E-59; Compound 116)

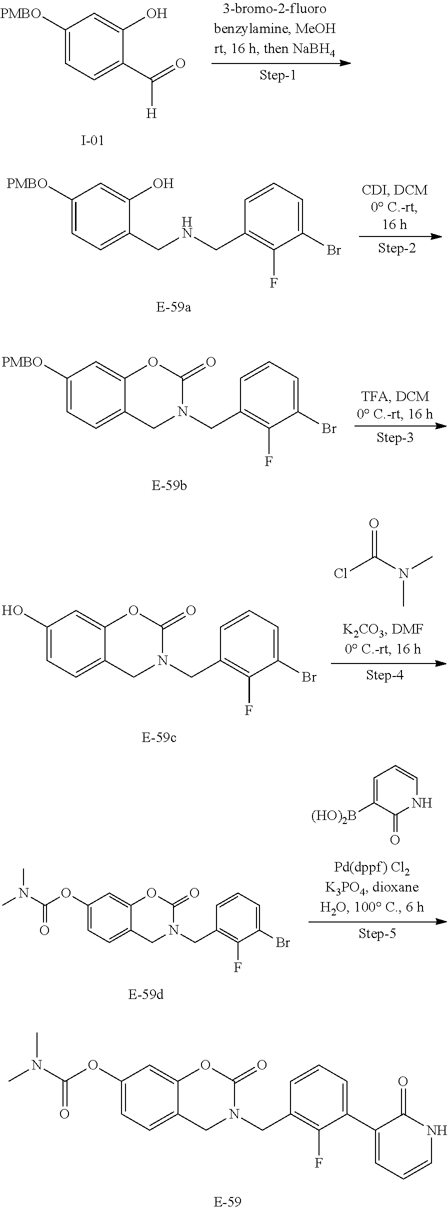

Step-1: Synthesis of 2-(((3-bromo-2-fluorobenzyl)amino)methyl)-5-((4-methoxybenzyl)oxy)phenol (E-59a): Compound E-59a (1.5 g) was synthesized by following procedure as described for the synthesis of E-01 (step-1) using 2-hydroxy-4-|(4-methoxyphenyl)methoxy]benzaldehyde (I-01) (1.3 g, 5.03 mmol), and 1-(3-bromo-2-fluorophenyl)methanamine (1.23 g, 6.04 mmol) as the starting materials. LCMS (ES) m/z: 446.1 [M+H]⁺.

Step-2: Synthesis of 3-(3-bromo-2-fluorobenzyl)-7-((4-methoxybenzyl)oxy)-3,4-dihydro-2H-benzo[e][1,3]oxazin-2-one (E-59b): Compound E-59b (1 g) was synthesized by following procedure as described for the synthesis of E-01 (step-2) using 2-({|(3-bromo-2-fluorophenyl)methyl]amino}methyl)-5-[(4-methoxy phenyl)methoxy]phenol (E-59a) (1.5 g, 3.36 mmol) as the starting material. LCMS (ES) m/z: 472.1 [M+H]⁺.

Step-3: Synthesis of 3-(3-bromo-2-fluorobenzyl)-7-hydroxy-3,4-dihydro-2H-benzo[e][1,3]oxazin-2-one (E-59c): To a stirred solution of 3-[(3-bromo-2-fluorophenyl) methyl]-7-[(4-methoxyphenyl)methoxy]-3,4-dihydro-2H-1,3-benzoxazin-2-one (E-59b) (1 g, 2.12 mmol) in DCM (10.7 mL) was added TFA (486 μL, 6.35 mmol) at 0° C. and the reaction mixture was slowly allowed to warm to room temperature. The reaction progress was monitored by TLC/LCMS. After completion of the reaction (16 h), volatiles were removed under reduced pressure and water (30 mL) was added to the residue. Extraction was carried out using EtOAc (3×30 mL); the combined organic extracts were washed with saturated NaHCO₃ solution (2×15 mL), brine solution (2×50 mL), dried over anhydrous Na₂SO₄, filtered and concentrated under reduced pressure. The residue was purified by Combi-Flash (using gradient elution of 0-5% of MeOH in DCM) to afford desired compound E-59c (0.59 g) as a pale yellow viscous oil. LCMS (ES) m/z: 352.1 [M+H]⁺.

Step-4: Synthesis of 3-(3-bromo-2-fluorobenzyl)-2-oxo-3,4-dihydro-2H-benzo[e][1,3]oxazin-7-yl dimethylcarbamate (E-59d): Compound E-59d (0.23 g) was synthesized by following procedure as described for the synthesis of E-27 (step-3) using 3-(3-bromo-2-fluorophenyl) methyl]-7-hydroxy-3,4-dihydro-2H-1,3-benzoxazin-2-one (E-59c) (210 mg, 596 μmol) as the starting material. LCMS (ES) m/z: 423.1 [M+H]⁺.

Step-5: Synthesis of 3-(2-fluoro-3-(2-oxo-1,2-dihydropyridin-3-yl)benzyl)-2-oxo-3,4-dihydro-2H-benzo[e][1,3]oxazin-7-yl dimethylcarbamate (E-59): To a stirred solution of 3-[(3-bromo-2-fluorophenyl)methyl]-2-oxo-3,4-dihydro-2H-1,3-benzoxazin-7-yl N,N-dimethylcarbamate (210 mg, 496 μmol) in 1,4-dioxane (4.38 mL) and water (876 μL) was added (2-oxo-1,2-dihydropyridin-3-yl)boronic acid (0.07 g, 546 μmol) and K₃PO₄ (0.31 g, 3.14 mmol). Argon gas was purged through it for 10 min before addition of Pd(dppf)Cl₂ (0.04 g, 4.96 mmol). The reaction mixture was then stirred at 100° C., while monitoring reaction progress by TLC. After complete consumption of starting material (6 h), the reaction mixture was cooled to room temperature and filtered through celite pad. The filtrate was concentrated under reduced pressure and water (10 mL) was added to the residue. Extraction was carried out using DCM (2×50 mL); the combined organic extracts were washed with brine solution (2×25 mL), dried over anhydrous Na₂SO₄, filtered and concentrated under reduced pressure. The residue was purified by Combi-Flash (using gradient elution of 0-9% of MeOH in DCM) followed by reverse phase preparative HPLC purification [using X-Select C18 (19 mm×250 mm, 5 μm particle size) column, 5 mM ammonium acetate in water and acetonitrile as mobile phase with 19 mL/min flow rate] to afford desired compound E-59 (40 mg) as an off-white solid. LCMS (ES) m/z: 438.3 [M+H]⁺. ¹H NMR (400 MHz, DMSO-d₆): δ 11.81 (s, 1H); 7.51 (d, J=1.2 Hz, 1H); 7.44-7.35 (m, 3H); 7.25-7.19 (m, 2H); 6.91-6.87 (m, 2H); 6.28 (t, J=6.8 Hz, 1H); 4.69 (s, 2H); 4.51 (s, 2H); 3.02 (s, 3H); 2.90 (s, 3H).

### Example 60: Preparation of 3-(2-fluoro-3-((N-methylsulfamoyl)methyl)benzyl)-2-oxo-3,4-dihydro-2H-benzo[e][1,3]oxazin-7-yl dimethylcarbamate (E-60; Compound 45)

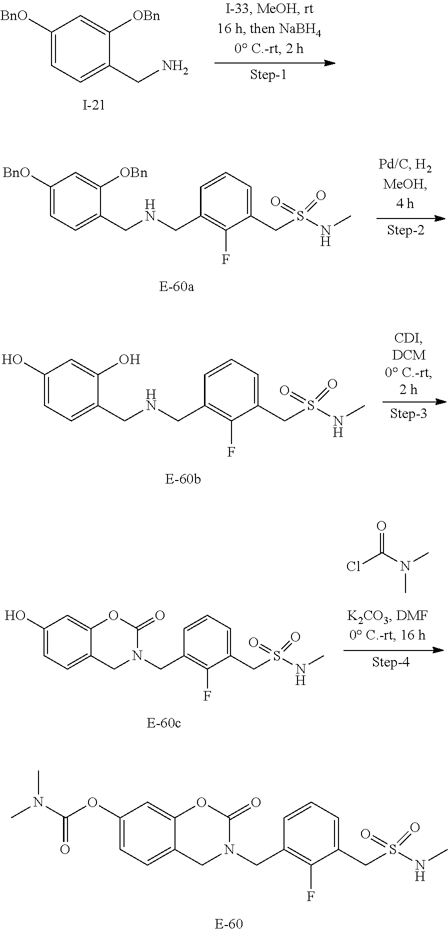

Step-1: Synthesis of 2-(((3-bromo-2-fluorobenzyl)amino)methyl)-5-((4-methoxybenzyl)oxy)phenol (E-60a): Compound E-60a (0.27 g) was synthesized by following procedure as described for the synthesis of E-01 (step-1) using 21-(2-fluoro-3-formylphenyl)-N-methylmethanesulfonamide (I-33) (0.14 g, 0.62 mmol) and (2,4-bis(benzyloxy)phenyl)methanamine (I-21) (0.2 g, 0.62 mmol) as the starting materials. LCMS (ES) m/z: 535.2 [M+H]⁺.

Step-2: Synthesis of 1-[3-({[(2,4-dihydroxyphenyl)methyl]amino}methyl)-2-fluorophenyl]-N-methylmethanesulfonamide (E-60b): To a stirred solution of 1-{3-[({[2,4-bis(benzyloxy)phenyl]methyl}amino)methyl]-2-fluorophenyl}-N-methylmethanesulfonamide (E-60a) (0.27 g, 0.50 mmol) in MeOH (10 mL) was added Pd/C (10%, 0.13 g, 1.26 mmol) and the reaction mixture was stirred under hydrogen atmosphere at room temperature. The reaction progress was monitored by TLC/LCMS analysis. After complete consumption of starting material (3 h), the catalyst was filtered off through a celite pad and washed with MeOH (3×5 mL). The filtrate was concentrated under reduced pressure and the residue was purified by Combi-Flash (using gradient elution of 0-5% of MeOH in DCM) to afford desired compound E-60b (0.15 g) as an off white solid. LCMS (ES) m/z: 355.1 [M+H]⁺.

Step-3: Synthesis of 1-{2-fluoro-3-[(7-hydroxy-2-oxo-3,4-dihydro-2H-1,3-benzoxazin-3-yl)methyl]phenyl}-N-methylmethanesulfonamide (E-60c): Compound E-60c (0.1 g) was synthesized by following procedure as described for the synthesis of E-01 (step-2) using 1-[3-({[(2,4-dihydroxyphenyl)methyl]amino}methyl)-2-fluorophenyl]-N-methylmethanesulfonamide (E-60b) (0.15 g, 0.42 mmol) as the starting material. LCMS (ES) m/z: 381.1 [M+H]⁺.

Step-4: Synthesis of 3-({2-fluoro-3-[(methylsulfamoyl)methyl]phenyl}methyl)-2-oxo-3,4-dihydro-2H-1,3-benzoxazin-7-yl N,N-dimethylcarbamate (E-60): Example E-60 (0.01 g) was synthesized by following procedure as described for the synthesis of E-01 (step-4) using 1-{2-fluoro-3-[(7-hydroxy-2-oxo-3,4-dihydro-2H-1,3-benzoxazin-3-yl)methyl]phenyl}-N-methylmethanesulfonamide (E-60c) (0.1 g, 0.26 mmol) as the starting material. The final compound was purified by preparative HPLC [using X-Select CSH C18 (19 mm×250 mm. 5 m particle size) column, 0.1% ammonia in water and acetonitrile as mobile phase with 18.0 mL/min flow rate]. LCMS (ES) m/z: 452.3 [M+H]⁺. ¹H NMR (400 MHz, DMSO-d₆): δ 7.44-7.38 (m, 2H); 7.24-7.20 (m, 2H); 7.09 (s, 1H); 6.91-6.87 (m, 2H); 4.68 (s, 2H); 4.49 (s, 2H); 4.38 (s, 2H); 3.02 (s, 3H); 2.90 (s, 3H); 2.58 (s, 3H).

### Examples 61 and 62: Preparations of (R)-3-(2-fluoro-3-((N-methylsulfamoyl)amino)benzyl)-4-methyl-2-oxo-3,4-dihydro-2H-benzo[e][1,3]oxazin-7-yl dimethylcarbamate and 3-(2-fluoro-3-((N-methylsulfamoyl)amino)benzyl)-4-methyl-2-oxo-3,4-dihydro-2H-benzo[e][1,3]oxazin-7-yl dimethylcarbamate and (S)-3-(2-fluoro-3-((N-methylsulfamoyl)amino)benzyl)-4-methyl-2-oxo-3,4-dihydro-2H-benzo[e][1,3]oxazin-7-yl dimethylcarbamate and 3-(2-fluoro-3-((N-methylsulfamoyl)amino)benzyl)-4-methyl-2-oxo-3,4-dihydro-2H-benzo[e][1,3]oxazin-7-yl dimethylcarbamate (E-61 and E-62; Compounds 152 and 153, respectively)

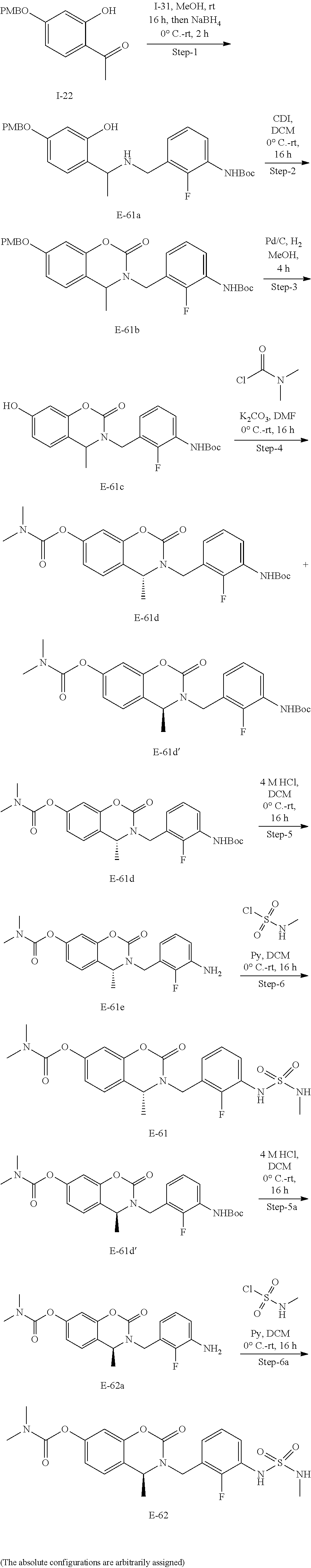

Step-1: Synthesis of tert-butyl (2-fluoro-3-(((1-(2-hydroxy-4-((4-methoxybenzyl)oxy)phenyl)ethyl)amino)methyl)phenyl)carbamate (E-61a): Compound E-61a (2 g) was synthesized by following procedure as described for the synthesis of E-01 (step-1) using 1-{2-hydroxy-4-[(4-methoxyphenyl)methoxy]phenyl}ethan-1-one (I-22) (2 g, 7.34 mmol) and tert-butyl N-[3-(aminomethyl)-2-fluorophenyl]carbamate (I-22) (1.76 g, 7.34 mmol) as the starting materials. LCMS (ES) m/z: 497.3 [M+H]⁺.

Step-2: Synthesis of tert-butyl (2-fluoro-3-((7-((4-methoxybenzyl)oxy)-4-methyl-2-oxo-2H-benzo[e][1,3]oxazin-3(4H)-yl)methyl)phenyl)carbamate (E-61b): Compound E-61b (1.9 g) was synthesized by following procedure as described for the synthesis of E-01 (step-2) using tert-butyl (2-fluoro-3-(((1-(2-hydroxy-4-((4-methoxybenzyl)oxy)phenyl)ethyl)amino)methyl)phenyl)carbamate (E-61a) (2 g, 4.03 mmol) as the starting material. LCMS (ES) m/z: 521.2 [M−H]⁺.

Step-3: Synthesis of tert-butyl (2-fluoro-3-((7-hydroxy-4-methyl-2-oxo-2H-benzo[e][1,3]oxazin-3(4H)-yl)methyl)phenyl)carbamate (E-61c): Compound E-61c (1.3 g) was synthesized by following procedure as described for the synthesis of E-01 (step-3) using tert-butyl N-[2-fluoro-3-({7-[(4-methoxyphenyl)methoxy]-4-methyl-2-oxo-3,4-dihydro-2H-1,3-benzoxazin-3-yl}methyl)phenyl]carbamate (E-61b) (1.9 g, 3.64 mmol) as the starting material. LCMS (ES) m/z: 401.2 [M−H]⁺.

Step-4: Synthesis of 3-(3-((tert-butoxycarbonyl)amino)-2-fluorobenzyl)-4-methyl-2-oxo-3,4-dihydro-2H-benzo[e][1,3]oxazin-7-yl dimethylcarbamate (E-61d): Compound 56d (0.25 g) and E-61d′ (0.25 g) were synthesized by following procedure as described for the synthesis of E-01 (step-4) using tert-butyl N-{2-fluoro-3-[(7-hydroxy-4-methyl-2-oxo-3,4-dihydro-2H-1,3-benzoxazin-3-yl)methyl]phenyl}carbamate (E-61c) (0.65 g, 1.62 mmol) as the starting material. The enantiomers were separated by chiral preparative HPLC [using Chiralpak IG (250 mm×20 mm, 5 m particle size) column, n-Hexane: IPA with 0.1% DEA (50:50) as mobile phase with 19.0 mL/min flow rate]. LCMS (ES) m/z: 474.2 [M+H]⁺.

Step-5: Synthesis of 3-(3-amino-2-fluorobenzyl)-4-methyl-2-oxo-3,4-dihydro-2H-benzo[e][1,3]oxazin-7-yl dimethylcarbamate (E-61e): Compound E-61e (0.18 g) was synthesized by following procedure as described for the synthesis of E-17 (step-3) using E-61d (250 mg, 0.52 mmol) as the starting material. LCMS (ES) m/z: 374.0 [M+H]⁺.

Step-5a: Synthesis of 3-(3-amino-2-fluorobenzyl)-4-methyl-2-oxo-3,4-dihydro-2H-benzo[e][1,3]oxazin-7-yl dimethylcarbamate (E-62a): Compound E-62a (0.18 g) was synthesized by following procedure as described for the synthesis of E-17 (step-3) using E-61d′ (250 mg, 0.52 mmol) as the starting material. LCMS (ES) m/z: 374.1 [M+H]⁺.

Step-6: Synthesis of 3-(2-fluoro-3-((N-methylsulfamoyl)amino)benzyl)-4-methyl-2-oxo-3,4-dihydro-2H-benzo[e][1,3]oxazin-7-yl dimethylcarbamate (E-61): Example E-61 (0.115 g) was synthesized by following procedure as described for the synthesis of E-17 (step-4) using E-61e (0.18 g, 0.48 mmol) as the starting material. The final compound was purified by reverse phase preparative HPLC purification [using Zorbax XDB C18 (150 mm×21.2 mm, 7 μm particle size) column, 0.1% ammonia in water and acetonitrile as mobile phase with 19.0 mL/min flow rate]. LCMS (ES) m/z: 467.2 [M+H]⁺. ¹H NMR (400 MHz, DMSO-d₆): δ 9.40 (s, 1H); 7.39-7.34 (m, 1H); 7.28 (d, J=8.0 Hz, 1H); 7.21 (d, J=4.8 Hz, 1H); 7.12 (t, J=8.0 Hz, 1H); 7.06 (t, J=6.4 Hz, 1H); 6.93-6.91 (m, 2H); 4.94 (d, J=15.6 Hz, 1H); 4.61-4.56 (m, 1H); 4.37 (d, J=16.0 Hz, 1H); 3.03 (s, 3H); 2.91 (s, 3H); 2.48 (d, J=4.8 Hz, 3H)); 1.40 (d, J=6.4 Hz, 3H). (note: absolute configuration was not determined).

Step-6a: Synthesis of 3-(2-fluoro-3-((N-methylsulfamoyl)amino)benzyl)-4-methyl-2-oxo-3,4-dihydro-2H-benzo[e][1,3]oxazin-7-yl dimethylcarbamate (E-62): Example E-62 (0.007 g) was synthesized by following procedure as described for the synthesis of E-17 (step-4) using E-62a (0.18 g, 0.48 mmol) as the starting material. The final compound was purified by reverse phase preparative HPLC purification [using X-Bridge C-18 (250 mm×4.6 mm×5 μm particle size) column, 0.1% ammonia in water and acetonitrile as mobile phase with 19.0 mL/min flow rate]. LCMS (ES) m/z: 467.2 [M+H]⁺. ¹H NMR (400 MHz, DMSO-d₆): δ 9.41 (s, 1H); 7.39-7.35 (m, 1H); 7.28 (d, J=8.0 Hz, 1H); 7.21 (d, J=4.4 Hz, 1H); 7.14-7.05 (m, 2H); 6.93-6.91 (m, 2H); 4.94 (d, J=15.6 Hz, 1H); 4.61-4.56 (m, 1H); 4.47 (d, J=15.6 Hz, 1H); 3.03 (s, 3H); 2.91 (s, 3H); 2.46 (d, J=4.8 Hz, 3H)); 1.40 (d, J=6.4 Hz, 3H). (note: absolute configuration was not determined).

### Example 63: Preparation of (R)-4-methyl-3-(3-((N-methylsulfamoyl)amino)benzyl)-2-oxo-3,4-dihydro-2H-benzo[e][1,3]oxazin-7-yl dimethyl carbamate or (S)-4-methyl-3-(3-((N-methylsulfamoyl)amino)benzyl)-2-oxo-3,4-dihydro-2H-benzo[e][1,3]oxazin-7-yl dimethyl carbamate (E-63; Compound 155)

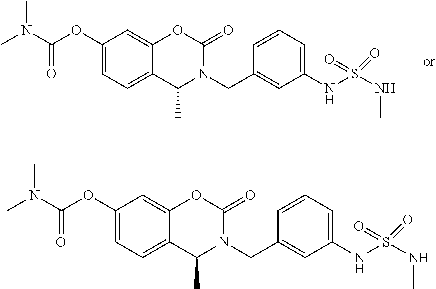

E-63 was synthesized by following similar synthesis sequence and experimental procedures as described for the synthesis of E-61, using key intermediates I-22 and I-31. LCMS (ES) m/z: 449.2 [M+H]⁺. ¹H NMR (400 MHz, DMSO-d₆): δ 9.64 (s, 1H); 7.26-7.22 (m, 3H); 7.09 (d, J=7.6 Hz, 2H); 6.92-6.89 (m, 3H); 4.85 (d, J=15.6 Hz, 1H); 4.56-4.51 (m, 1H); 4.38 (d, J=15.6 Hz, 1H); 3.02 (s, 3H); 2.90 (s, 3H); 2.40 (d, J=5.2 Hz, 3H); 1.35 (d, J=6.4 Hz, 3H).

### Example 64: Preparation of (S)-4-methyl-3-(3-((N-methylsulfamoyl)amino)benzyl)-2-oxo-3,4-dihydro-2H-benzo[e][1,3]oxazin-7-yl dimethylcarbamate or (R)-4-methyl-3-(3-((N-methylsulfamoyl)amino)benzyl)-2-oxo-3,4-dihydro-2H-benzo[e][1,3]oxazin-7-yl dimethylcarbamate (E-64; Compound 154)

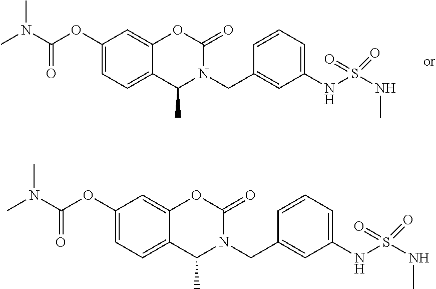

E-64 was synthesized by following similar synthesis sequence and experimental procedures as described for the synthesis of E-61, using key intermediates I-22 and I-31. LCMS (ES) m/z: 449.2 [M+H]⁺. ¹H NMR (400 MHz, DMSO-d₆): δ 9.66 (s, 1H); 7.27-7.23 (m, 3H); 7.10 (d, J=7.6 Hz, 2H); 6.93-6.90 (m, 3H); 4.86 (d, J=15.6 Hz, 1H); 4.55-4.54 (m, 1H); 4.38 (d, J=15.6 Hz, 1H); 3.03 (s, 3H); 2.91 (s, 3H); 2.40 (d, J=5.2 Hz, 3H); 1.35 (d, J=6.4 Hz, 3H).

### Examples 65 and 66: Preparations of (R)-3-{[2-fluoro-3-(methylaminosulfonylamino)phenyl]methyl}-4-methyl-7-(2-pyrimidinyloxy)-3,4-dihydro-2H-1,3-benzoxazin-2-one and (S)-3-{[2-fluoro-3-(methylaminosulfonylamino)phenyl]methyl}-4-methyl-7-(2-pyrimidinyloxy)-3,4-dihydro-2H-1,3-benzoxazin-2-one (E-65 and E-66; Compounds 150 and 151)

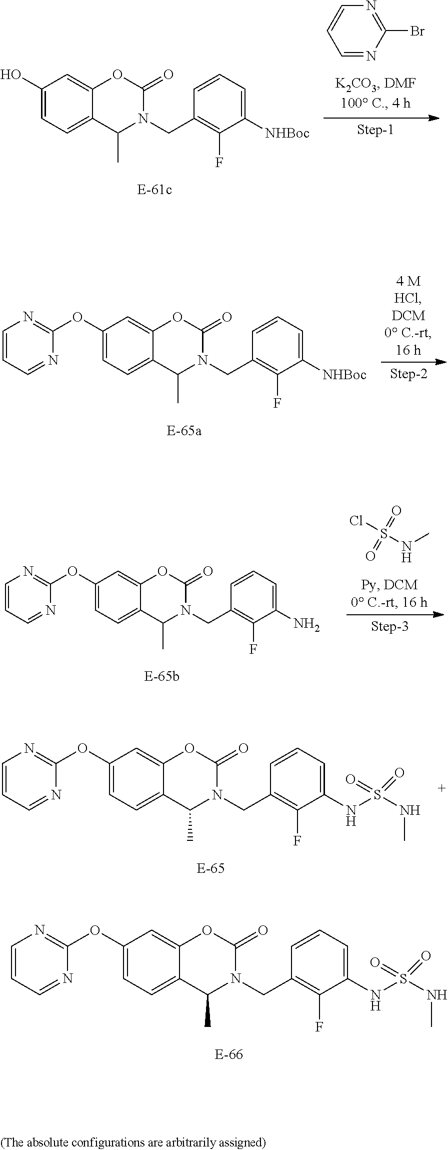

Step-1: Synthesis of tert-butyl (2-fluoro-3-((4-methyl-2-oxo-7-(pyrimidin-2-yloxy)-2H-benzo[e][1,3]oxazin-3(4H)-yl)methyl)phenyl)carbamate (E-65a): Compound E-65a (0.65 g) was synthesized by following procedure as described for the synthesis of E-50 (step-1) using tert-butyl (2-fluoro-3-((7-hydroxy-4-methyl-2-oxo-2H-benzo[e][1,3]oxazin-3(4H)-yl)methyl)phenyl)carbamate (E-61c) (0.65 g, 1.62 mmol) and 2-bromopyrimidine (0.28 g, 1.78 mmol) as the starting materials. LCMS (ES) m/z: 479.1 [M−H]⁺.

Step-2: Synthesis of 3-(3-amino-2-fluorobenzyl)-4-methyl-7-(pyrimidin-2-yloxy)-3,4-dihydro-2H-benzo[e][1,3]oxazin-2-one (E-65b): Compound E-65b (0.45 g) was synthesized by following procedure as described for the synthesis of E-17 (step-3) using tert-butyl (2-fluoro-3-((4-methyl-2-oxo-7-(pyrimidin-2-yloxy)-2H-benzo[e][1,3]oxazin-3(4H)-yl)methyl)phenyl)carbamate (E-65a) (0.6 g, 1.25 mmol) as the starting material. LCMS (ES) m/z: 381.2 [M+H]⁺.

Step-3: Synthesis of 3-{[2-fluoro-3-(methylaminosulfonylamino)phenyl]methyl}-4-methyl-7-(2-pyrimidinyloxy)-3,4-dihydro-2H-1,3-benzoxazin-2-one (E-65): Example E-65 (0.075 g) and E-66 (0.065 g) were synthesized by following procedure as described for the synthesis of E-17 (step-4) using 3-(3-amino-2-fluorobenzyl)-4-methyl-7-(pyrimidin-2-yloxy)-3,4-dihydro-2H-benzo[e][1,3]oxazin-2-one (E-65b) (0.45 g, 1.18 mmol) as the starting material. Both enantiomers were separated by chiral Prep-HPLC [using Chiralpak IG (250 mm×20 mm, 5 m particle size) column, 0.1% DEA and methanol as mobile phase with 12.0 mL/min flow rate].

Example 65: LCMS (ES) m/z: 474.2 [M+H]⁺. ¹H NMR (400 MHz, DMSO d₆): 6 9.42 (s, 1H); 8.63 (d, J=4.8 Hz, 2H); 7.37-7.33 (m, 2H); 7.27 (t, J=4.8 Hz, 1H); 7.20-7.19 (m, 1H); 7.13-7.05 (m, 2H); 7.02-6.97 (m, 2H); 4.93 (d, J=15.6 Hz, 1H); 4.60-4.57 (m, 1H); 4.45 (d, J=15.6 Hz, 1H); 2.43 (s, 3H); 1.41 (d, J=6.4 Hz, 3H).

Example 66: LCMS (ES) m/z: 474.2 [M+H]⁺. ¹H NMR (400 MHz, DMSO d₆): δ 9.41 (s, 1H); 8.63 (d, J=4.8 Hz, 2H); 7.37-7.26 (m, 3H); 7.15-6.97 (m, 5H); 4.93 (d, J=15.6 Hz, 1H); 4.61-4.57 (m, 1H); 4.45 (d, J=15.6 Hz, 1H); 2.43 (s, 3H); 1.41 (d, J=6.4 Hz, 3H). Note: Absolute configuration of both the enantiomers was not determined.

### Example 67: Preparation of (R)-8-fluoro-3-(2-fluoro-3-((N-methylsulfamoyl)amino)benzyl)-4-methyl-2-oxo-3,4-dihydro-2H-benzo[e][1,3]oxazin-7-yl dimethylcarbamate or (S)-8-fluoro-3-(2-fluoro-3-((N-methylsulfamoyl)amino)benzyl)-4-methyl-2-oxo-3,4-dihydro-2H-benzo[e][1,3]oxazin-7-yl dimethylcarbamate (E-67; Compound 104)

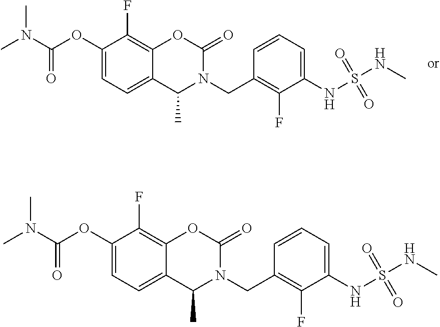

E-67 was synthesized by following similar synthesis sequence and experimental procedures as described for the synthesis of E-65, using key intermediates I-23 and I-31. LCMS (ES) m/z: 485.2 [M+H]⁺. ¹H NMR (400 MHz, DMSO-d₆): δ 9.39 (s, 1H); 7.39-7.35 (m, 1H); 7.22-7.18 (m, 1H); 7.14-7.04 (m, 4H); 4.94 (d, J=15.6 Hz, 1H); 4.65 (q, J=6.4 Hz, 1H); 4.50 (d, J=15.6 Hz, 1H); 3.06 (s, 3H); 2.92 (s, 3H); 2.47 (d, J=4.8 Hz, 3H); 1.43 (d, J=6.4 Hz, 3H). Note: Absolute configuration not determined.

### Example 68: Preparation of (S)-8-fluoro-3-(2-fluoro-3-((N-methylsulfamoyl)amino)benzyl)-4-methyl-2-oxo-3,4-dihydro-2H-benzo[e][1,3]oxazin-7-yl dimethylcarbamate or (R)-8-fluoro-3-(2-fluoro-3-((N-methylsulfamoyl)amino)benzyl)-4-methyl-2-oxo-3,4-dihydro-2H-benzo[e][1,3]oxazin-7-yl dimethylcarbamate (E-68; Compound 103)

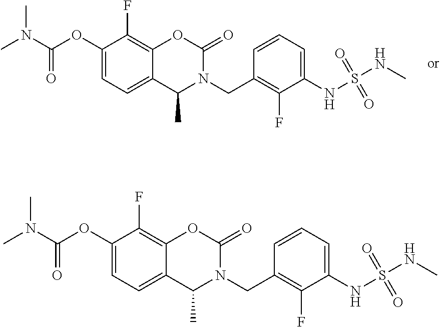

E-68 was synthesized by following similar synthesis sequence and experimental procedures as described for the synthesis of E-65, using key intermediates I-23 and I-31. LCMS (ES) m/z: 485.2 [M+H]⁺. ¹H NMR (400 MHz, DMSO-d₆): δ 9.39 (s, 1H); 7.39-7.35 (m, 1H); 7.22-7.18 (m, 1H); 7.14-7.04 (m, 4H); 4.94 (d, J=15.6 Hz, 1H); 4.65 (q, J=6.4 Hz, 1H); 4.50 (d, J=15.6 Hz, 1H); 3.06 (s, 3H); 2.92 (s, 3H); 2.47 (d, J=4.8 Hz, 3H); 1.43 (d, J=6.4 Hz, 3H). Note: Absolute configuration not determined.

### Example 69: Preparation of (R)-8-fluoro-3-({2-fluoro-3-(methylsulfamoyl)amino]phenyl}methyl)-4-methyl-7-(pyrimidin-2-yloxy)-3,4-dihydro-2H-1,3-benzoxazin-2-one or (S)-8-fluoro-3-({2-fluoro-3-(methylsulfamoyl)amino]phenyl}methyl)-4-methyl-7-(pyrimidin-2-yloxy)-3,4-dihydro-2H-1,3-benzoxazin-2-one (E-69; Compound 93)

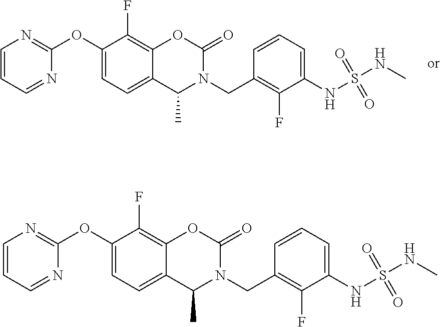 [SMILES: CNS(=O)(=O)Nc1cccc(CN2C(=O)Oc3c(ccc(Oc4ncccn4)c3F)[C@H]2C)c1F ; CNS(=O)(=O)Nc1cccc(CN2C(=O)Oc3c(ccc(Oc4ncccn4)c3F)[C@@H]2C)c1F]

E-69 was synthesized by following similar synthesis sequence and experimental procedures as described for the synthesis of E-65, using key intermediates I-23 and I-31. LCMS (ES) m/z: 492.3 [M+H]⁺. ¹H NMR (400 MHz, DMSO-d₆): δ 9.57 (s, 1H); 8.69 (d, J=4.8 Hz, 2H); 7.40-7.32 (m, 2H); 7.20-7.16 (m, 2H); 7.10-7.04 (m, 2H); 6.92 (s, 1H); 4.94 (d, J=15.6 Hz, 1H); 4.68 (q, J=6.4 Hz, 1H); 4.50 (d, J=15.6 Hz, 1H); 2.47 (d, J=4.8 Hz, 3H); 1.46 (d, J=6.4 Hz, 3H). Note: Absolute configuration not determined.

### Example 70: Preparation of (S)-8-fluoro-3-({2-fluoro-3-(methylsulfamoyl)amino]phenyl}methyl)-4-methyl-7-(pyrimidin-2-yloxy)-3,4-dihydro-2H-1,3-benzoxazin-2-one or (R)-8-fluoro-3-({2-fluoro-3-(methylsulfamoyl)amino]phenyl}methyl)-4-methyl-7-(pyrimidin-2-yloxy)-3,4-dihydro-2H-1,3-benzoxazin-2-one (E-70; Compound 92)

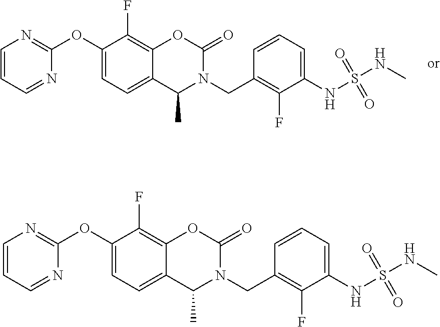 [SMILES: CNS(=O)(=O)Nc1cccc(CN2C(=O)Oc3c(ccc(Oc4ncccn4)c3F)[C@@H]2C)c1F ; CNS(=O)(=O)Nc1cccc(CN2C(=O)Oc3c(ccc(Oc4ncccn4)c3F)[C@H]2C)c1F]

E-70 was synthesized by following similar synthesis sequence and experimental procedures as described for the synthesis of E-65, using key intermediates I-23 and I-31. LCMS (ES) m/z: 492.3 [M+H]⁺. ¹H NMR (400 MHz, DMSO-d₆): δ 9.35 (s, 1H); 8.69 (d, J=4.8 Hz, 2H); 7.42-7.32 (m, 2H); 7.22-7.10 (m, 5H); 4.94 (d, J=15.6 Hz, 1H); 4.69 (q, J=6.8 Hz, 1H); 4.52 (d, J=15.6 Hz, 1H); 2.47 (d, J=4.8 Hz, 3H); 1.47 (d, J=6.8 Hz, 3H). Note: Absolute configuration not determined.

### Example 71: Preparation of (R)-6-fluoro-3-({2-fluoro-3-(methylsulfamoyl)amino]phenyl}methyl)-4-methyl-7-(1,3-oxazol-2-yloxy)-3,4-dihydro-2H-1,3-benzoxazin-2-one or (S)-6-fluoro-3-({2-fluoro-3-(methylsulfamoyl)amino]phenyl}methyl)-4-methyl-7-(1,3-oxazol-2-yloxy)-3,4-dihydro-2H-1,3-benzoxazin-2-one (E-71; Compound 85)

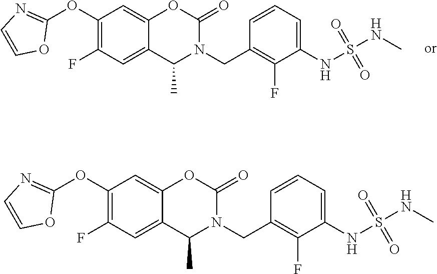 [SMILES: CNS(=O)(=O)Nc1cccc(CN2C(=O)Oc3cc(Oc4ncco4)c(F)cc3[C@H]2C)c1F ; CNS(=O)(=O)Nc1cccc(CN2C(=O)Oc3cc(Oc4ncco4)c(F)cc3[C@@H]2C)c1F]

E-71 was synthesized by following similar synthesis sequence and experimental procedures as described for the synthesis of E-65, using key intermediates I-24 and I-31. LCMS (ES) m/z: 481.3 [M+H]⁺. ¹H NMR (400 MHz, DMSO-d₆): δ 9.38 (s, 1H); 7.85 (d, J=0.8 Hz 1H); 7.52-7.48 (m, 2H); 7.40-7.32 (m, 1H); 7.22-7.06 (m, 3H); 7.02 (d, J=1.2 Hz, 1H); 4.95 (d, J=15.6 Hz, 1H); 4.60 (q, J=6.4 Hz, 1H); 4.48 (d, J=15.6 Hz, 1H); 2.49 (s, 3H); 1.43 (d, J=6.8 Hz, 3H). Note: Absolute configuration not determined.

### Example 72: Preparation of (S)-6-fluoro-3-({2-fluoro-3-(methylsulfamoyl)amino]phenyl}methyl)-4-methyl-7-(1,3-oxazol-2-yloxy)-3,4-dihydro-2H-1,3-benzoxazin-2-one or (R)-6-fluoro-3-({2-fluoro-3-(methylsulfamoyl)amino]phenyl}methyl)-4-methyl-7-(1,3-oxazol-2-yloxy)-3,4-dihydro-2H-1,3-benzoxazin-2-one (E-72; Compound 84)

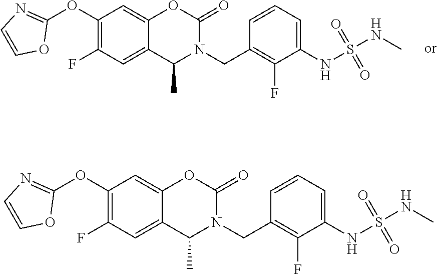 [SMILES: CNS(=O)(=O)Nc1cccc(CN2C(=O)Oc3cc(Oc4ncco4)c(F)cc3[C@@H]2C)c1F ; CNS(=O)(=O)Nc1cccc(CN2C(=O)Oc3cc(Oc4ncco4)c(F)cc3[C@H]2C)c1F]

E-72 was synthesized by following similar synthesis sequence and experimental procedures as described for the synthesis of E-65, using key intermediates I-24 and I-31. LCMS (ES) m/z: 481.3 [M+H]⁺. ¹H NMR (400 MHz, DMSO-d₆): δ 9.36 (br s, 1H); 7.84 (d, J=0.8 Hz 1H); 7.52-7.48 (m, 2H); 7.40-7.36 (m, 1H); 7.22-7.06 (m, 3H); 7.02 (d, J=0.8 Hz, 1H); 4.94 (d, J=15.6 Hz, 1H); 4.60 (q, J=6.4 Hz, 1H); 4.48 (d, J=16 Hz, 1H); 2.49 (d, J=4.8 Hz, 3H); 1.43 (d, J=6.8 Hz, 3H). Note: Absolute configuration not determined.

### Example 73: Preparation of (R)-5-fluoro-3-({2-fluoro-3-(methylsulfamoyl)amino]phenyl}methyl)-4-methyl-7-(1,3-oxazol-2-yloxy)-3,4-dihydro-2H-1,3-benzoxazin-2-one or (S)-5-fluoro-3-({2-fluoro-3-(methylsulfamoyl)amino]phenyl}methyl)-4-methyl-7-(1,3-oxazol-2-yloxy)-3,4-dihydro-2H-1,3-benzoxazin-2-one (E-73; Compound 87)

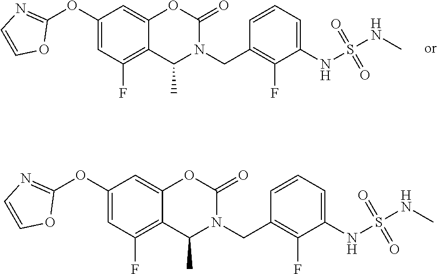 [SMILES: CNS(=O)(=O)Nc1cccc(CN2C(=O)Oc3cc(Oc4ncco4)cc(F)c3[C@H]2C)c1F ; CNS(=O)(=O)Nc1cccc(CN2C(=O)Oc3cc(Oc4ncco4)cc(F)c3[C@@H]2C)c1F]

E-73 was synthesized by following similar synthesis sequence and experimental procedures as described for the synthesis of E-65, using key intermediates I-25 and I-31. LCMS (ES) m/z: 481.3 [M+H]⁺. ¹H NMR (400 MHz, DMSO-d₆): δ 9.39 (s, 1H); 7.85 (d, J=0.8 Hz, 1H); 7.40-7.35 (m, 1H); 7.32-7.28 (m, 1H); 7.24-7.16 (m, 2H); 7.13-7.09 (m, 2H); 7.06 (d, J=0.8 Hz, 1H); 4.92 (d, J=15.6 Hz, 1H); 4.77 (q, J=6.4 Hz, 1H); 4.54 (d, J=15.6 Hz, 1H); 2.48 (d, J=4.8 Hz, 3H); 1.44 (d, J=6.4 Hz, 3H). Note: Absolute configuration not determined.

### Example 74: Preparation of (S)-5-fluoro-3-({2-fluoro-3-(methylsulfamoyl)amino]phenyl}methyl)-4-methyl-7-(1,3-oxazol-2-yloxy)-3,4-dihydro-2H-1,3-benzoxazin-2-one or (R)-5-fluoro-3-({2-fluoro-3-(methylsulfamoyl)amino]phenyl}methyl)-4-methyl-7-(1,3-oxazol-2-yloxy)-3,4-dihydro-2H-1,3-benzoxazin-2-one (E-74; Compound 86)

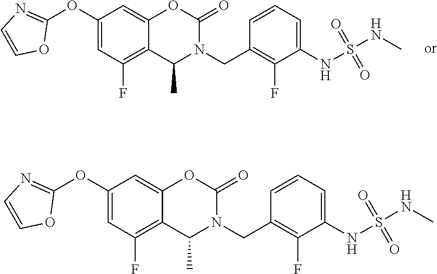 [SMILES: CNS(=O)(=O)Nc1cccc(CN2C(=O)Oc3cc(Oc4ncco4)cc(F)c3[C@@H]2C)c1F ; CNS(=O)(=O)Nc1cccc(CN2C(=O)Oc3cc(Oc4ncco4)cc(F)c3[C@H]2C)c1F]

E-74 was synthesized by following similar synthesis sequence and experimental procedures as described for the synthesis of E-65, using key intermediates I-25 and I-31. LCMS (ES) m/z: 481.3 [M+H]⁺. ¹H NMR (400 MHz, DMSO-d₆): (9.39 (s, 1H); 7.85 (d, J=0.8 Hz, 1H); 7.40-7.35 (m, 1H); 7.32-7.28 (m, 1H); 7.24-7.16 (m, 2H); 7.13-7.09 (m, 2H); 7.06 (d, J=0.8 Hz, 1H); 4.92 (d, J=15.6 Hz, 1H); 4.77 (q, J=6.4 Hz, 1H); 4.54 (d, J=15.6 Hz, 1H); 2.48 (d, J=4.8 Hz, 3H); 1.44 (d, J=6.4 Hz, 3H). Note: Absolute configuration not determined.

### Example 75: Preparation of (R)-8-fluoro-3-({2-fluoro-3-(methylsulfamoyl)amino]phenyl}methyl)-4-methyl-7-(1,3-oxazol-2-yloxy)-3,4-dihydro-2H-1,3-benzoxazin-2-one or (S)-8-fluoro-3-({2-fluoro-3-(methylsulfamoyl)amino]phenyl}methyl)-4-methyl-7-(1,3-oxazol-2-yloxy)-3,4-dihydro-2H-1,3-benzoxazin-2-one (E-75; Compound 90)

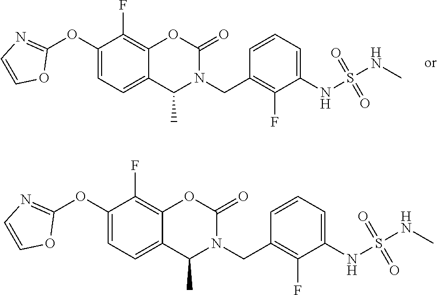 [SMILES: CNS(=O)(=O)Nc1cccc(CN2C(=O)Oc3c(ccc(Oc4ncco4)c3F)[C@H]2C)c1F ; CNS(=O)(=O)Nc1cccc(CN2C(=O)Oc3c(ccc(Oc4ncco4)c3F)[C@@H]2C)c1F]

E-75 was synthesized by following similar synthesis sequence and experimental procedures as described for the synthesis of E-65, using key intermediates I-23 and I-31. LCMS (ES) m/z: 481.3 [M+H]⁺. ¹H NMR (400 MHz, DMSO-d₆): δ 9.38 (s, 1H); 7.85 (d, J=0.8 Hz, 1H); 7.40-7.34 (m, 2H); 7.24-7.10 (m, 4H); 7.02 (d, J=1.2 Hz, 1H); 4.93 (d, J=15.6 Hz, 1H); 4.69 (q, J=6.4 Hz, 1H); 4.51 (d, J=15.6 Hz, 1H); 2.48 (d, J=4.8 Hz, 3H); 1.45 (d, J=6.4 Hz, 3H). Note: Absolute configuration not determined.

### Example 76: Preparation of (S)-8-fluoro-3-({2-fluoro-3-(methylsulfamoyl)amino]phenyl}methyl)-4-methyl-7-(1,3-oxazol-2-yloxy)-3,4-dihydro-2H-1,3-benzoxazin-2-one or (R)-8-fluoro-3-({2-fluoro-3-(methylsulfamoyl)amino]phenyl}methyl)-4-methyl-7-(1,3-oxazol-2-yloxy)-3,4-dihydro-2H-1,3-benzoxazin-2-one (E-76; Compound 89)

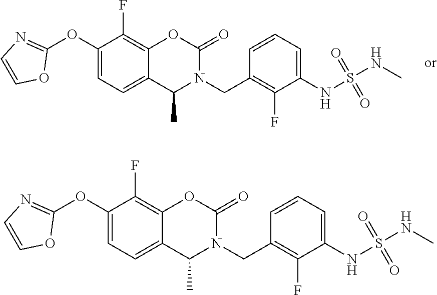 [SMILES: CNS(=O)(=O)Nc1cccc(CN2C(=O)Oc3c(ccc(Oc4ncco4)c3F)[C@@H]2C)c1F ; CNS(=O)(=O)Nc1cccc(CN2C(=O)Oc3c(ccc(Oc4ncco4)c3F)[C@H]2C)c1F]

E-76 was synthesized by following similar synthesis sequence and experimental procedures as described for the synthesis of E-65, using key intermediates I-23 and I-31. LCMS (ES) m/z: 481.3 [M+H]⁺. ¹H NMR (400 MHz, DMSO-d₆): δ 9.38 (s, 1H); 7.85 (d, J=0.8 Hz, 1H); 7.40-7.34 (m, 2H); 7.24-7.10 (m, 4H); 7.02 (d, J=1.2 Hz, 1H); 4.93 (d, J=15.6 Hz, 1H); 4.69 (q, J=6.4 Hz, 1H); 4.51 (d, J=15.6 Hz, 1H); 2.48 (d, J=4.8 Hz, 3H); 1.45 (d, J=6.4 Hz, 3H). Note: Absolute configuration not determined.

### Example 77: Preparation of (R)-3-({2-fluoro-3-(methylsulfamoyl)amino]phenyl}methyl)-4-methyl-7-(1,3-oxazol-2-yloxy)-3,4-dihydro-2H-1,3-benzoxazin-2-one or (S)-3-({2-fluoro-3-(methylsulfamoyl)amino]phenyl}methyl)-4-methyl-7-(1,3-oxazol-2-yloxy)-3,4-dihydro-2H-1,3-benzoxazin-2-one (E-77; Compound 79)

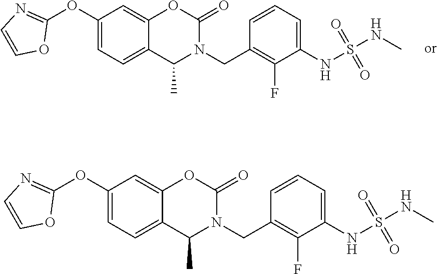 [SMILES: CNS(=O)(=O)Nc1cccc(CN2C(=O)Oc3cc(Oc4ncco4)ccc3[C@H]2C)c1F ; CNS(=O)(=O)Nc1cccc(CN2C(=O)Oc3cc(Oc4ncco4)ccc3[C@@H]2C)c1F]

E-77 was synthesized by following similar synthesis sequence and experimental procedures as described for the synthesis of E-65, using key intermediates I-22 and I-31. LCMS (ES) m/z: 463.3 [M+H]⁺. ¹H NMR (400 MHz, DMSO-d₆): δ 9.37 (s, 1H); 7.83 (d, J=0.8 Hz, 1H); 7.39-7.34 (m, 2H); 7.23 (d, J=2.4 Hz, 1H); 7.20-7.04 (m, 4H); 7.03 (d, J=1.2 Hz, 1H); 4.94 (d, J=16.0 Hz, 1H); 4.62 (q, J=6.4 Hz, 1H); 4.47 (d, J=16.0 Hz, 1H); 2.48 (d, J=4.8 Hz, 3H); 1.42 (d, J=6.4 Hz, 3H). Note: Absolute configuration not determined.

### Example 78: Preparation of (S)-3-({2-fluoro-3-(methylsulfamoyl)amino]phenyl}methyl)-4-methyl-7-(1,3-oxazol-2-yloxy)-3,4-dihydro-2H-1,3-benzoxazin-2-one or (R)-3-({2-fluoro-3-(methylsulfamoyl)amino]phenyl}methyl)-4-methyl-7-(1,3-oxazol-2-yloxy)-3,4-dihydro-2H-1,3-benzoxazin-2-one (E-78; Compound 78)

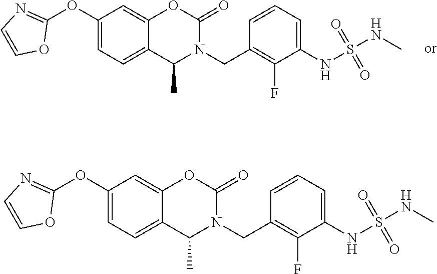 [SMILES: CNS(=O)(=O)Nc1cccc(CN2C(=O)Oc3cc(Oc4ncco4)ccc3[C@@H]2C)c1F ; CNS(=O)(=O)Nc1cccc(CN2C(=O)Oc3cc(Oc4ncco4)ccc3[C@H]2C)c1F]

E-78 was synthesized by following similar synthesis sequence and experimental procedures as described for the synthesis of E-65, using key intermediates I-22 and I-31. LCMS (ES) m/z: 463.3 [M+H]⁺. ¹H NMR (400 MHz, DMSO-d₆): δ 9.35 (s, 1H); 7.83 (d, J=0.8 Hz, 1H); 7.39-7.34 (m, 2H); 7.23 (d, J=2.4 Hz, 1H); 7.19-7.04 (m, 4H); 7.03 (d, J=1.2 Hz, 1H); 4.94 (d, J=15.6 Hz, 1H); 4.62 (q, J=6.4 Hz, 1H); 4.47 (d, J=15.6 Hz, 1H); 2.48 (d, J=4.8 Hz, 3H); 1.41 (d, J=6.4 Hz, 3H). Note: Absolute configuration not determined.

### Example 79: Preparation of (R)-3-({2-fluoro-3-(methylsulfamoyl)amino]phenyl}methyl)-4-methyl-7-(pyridazin-3-yloxy)-3,4-dihydro-2H-1,3-benzoxazin-2-one or (S)-3-({2-fluoro-3-(methylsulfamoyl)amino]phenyl}methyl)-4-methyl-7-(pyridazin-3-yloxy)-3,4-dihydro-2H-1,3-benzoxazin-2-one (E-79; Compound 72)

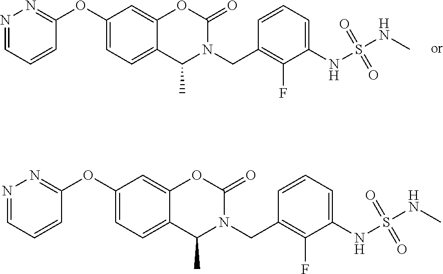 [SMILES: CNS(=O)(=O)Nc1cccc(CN2C(=O)Oc3cc(Oc4cccnn4)ccc3[C@H]2C)c1F ; CNS(=O)(=O)Nc1cccc(CN2C(=O)Oc3cc(Oc4cccnn4)ccc3[C@@H]2C)c1F]

E-79 was synthesized by following similar synthesis sequence and experimental procedures as described for the synthesis of E-65, using key intermediates I-22 and I-31. LCMS (ES) m/z: 474.3 [M+H]⁺. ¹H NMR (400 MHz, DMSO-d₆): δ 9.39 (s, 1H); 9.03 (dd, J₁=1.2 Hz, J₂=4.4 Hz, 1H); 7.80-7.86 (m, 1H); 7.49 (dd, J₁=1.2 Hz, 12=8.8 Hz, 1H); 7.40-7.35 (m, 2H); 7.22-6.98 (m, 5H); 4.96 (d, J=15.6, 1H); 4.62 (q, J=6.4 Hz, 1H); 4.48 (d, J=15.6 Hz, 1H); 2.49 (d, J=4.8 Hz, 3H); 1.44 (d, J=6.4 Hz, 3H). Note: Absolute configuration not determined.

### Example 80: Preparation of (S)-3-({2-fluoro-3-(methylsulfamoyl)amino]phenyl}methyl)-4-methyl-7-(pyridazin-3-yloxy)-3,4-dihydro-2H-1,3-benzoxazin-2-one or (R)-3-({2-fluoro-3-(methylsulfamoyl)amino]phenyl}methyl)-4-methyl-7-(pyridazin-3-yloxy)-3,4-dihydro-2H-1,3-benzoxazin-2-one (E-80; Compound 71)

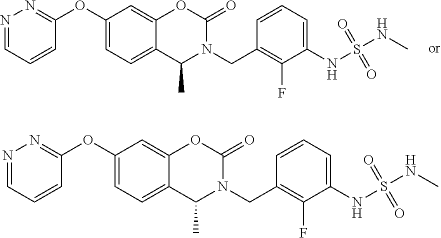 [SMILES: CNS(=O)(=O)Nc1cccc(CN2C(=O)Oc3cc(Oc4cccnn4)ccc3[C@@H]2C)c1F ; CNS(=O)(=O)Nc1cccc(CN2C(=O)Oc3cc(Oc4cccnn4)ccc3[C@H]2C)c1F]

E-80 was synthesized by following similar synthesis sequence and experimental procedures as described for the synthesis of E-65, using key intermediates I-22 and I-31. LCMS (ES) m/z: 474.3 [M+H]⁺. ¹H NMR (400 MHz, DMSO-d₆): (9.38 (s, 1H); 9.03 (dd, J₁=1.2 Hz, J₂=4.4 Hz, 1H); 7.80-7.86 (m, 1H); 7.49 (dd, J₁=1.2 Hz, J₂=8.8 Hz, 1H); 7.40-7.32 (m, 2H); 7.22-7.00 (m, 5H); 4.96 (d, J=15.6, 1H); 4.62 (q, J=6.4 Hz, 1H); 4.48 (d, J=15.6 Hz, 1H); 2.49 (d, J=4.8 Hz, 3H); 1.44 (d, J=6.8 Hz, 3H). Note: Absolute configuration not determined.

### Example 81: Preparation of (R)-6-fluoro-3-({2-fluoro-3-(methylsulfamoyl)amino]phenyl}methyl)-4-methyl-7-(pyridazin-3-yloxy)-3,4-dihydro-2H-1,3-benzoxazin-2-one or (S)-6-fluoro-3-({2-fluoro-3-(methylsulfamoyl)amino]phenyl}methyl)-4-methyl-7-(pyridazine-3-yloxy)-3,4-dihydro-2H-1,3-benzoxazin-2-one (E-81; Compound 67)

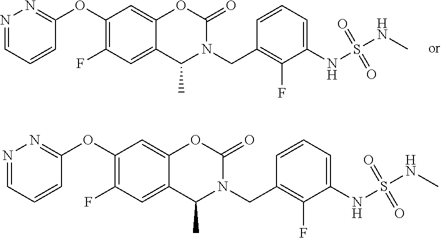 [SMILES: CNS(=O)(=O)Nc1cccc(CN2C(=O)Oc3cc(Oc4cccnn4)c(F)cc3[C@H]2C)c1F ; CNS(=O)(=O)Nc1cccc(CN2C(=O)Oc3cc(Oc4cccnn4)c(F)cc3[C@@H]2C)c1F]

E-81 was synthesized by following similar synthesis sequence and experimental procedures as described for the synthesis of E-65, using key intermediates I-24 and I-31. LCMS (ES) m/z: 490.3 [M−H]⁺. ¹H NMR (400 MHz, DMSO-d₆): δ 9.41 (s, 1H); 9.04 (d, J=4.0 Hz, 1H), 7.84-7.80 (m, 1H); 7.61 (dd, J₁=1.2 Hz, J₂=9.2 Hz, 1H); 7.47 (d, J=10.4 Hz, 1H); 7.40-7.34 (m, 1H); 7.31 (d, J=6.4 Hz, 1H); 7.18-7.02 (m, 3H); 4.96 (d, J=15.6, 1H); 4.61 (q, J=6.4 Hz, 1H); 4.48 (d, J=15.6 Hz, 1H); 2.50 (d, J=4.8 Hz, 3H); 1.46 (d, J=6.4 Hz, 3H). Note: Absolute configuration not determined.

### Example 82: Preparation of (S)-6-fluoro-3-({2-fluoro-3-(methylsulfamoyl)amino]phenyl}methyl)-4-methyl-7-(pyridazin-3-yloxy)-3,4-dihydro-2H-1,3-benzoxazin-2-one or (R)-6-fluoro-3-({2-fluoro-3-(methylsulfamoyl)amino]phenyl}methyl)-4-methyl-7-(pyridazin-3-yloxy)-3,4-dihydro-2H-1,3-benzoxazin-2-one (E-82; Compound 66)

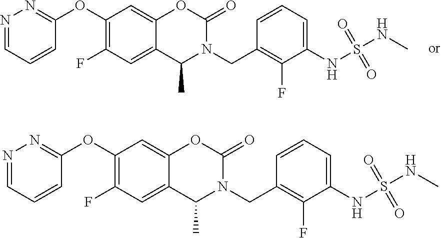 [SMILES: CNS(=O)(=O)Nc1cccc(CN2C(=O)Oc3cc(Oc4cccnn4)c(F)cc3[C@@H]2C)c1F ; CNS(=O)(=O)Nc1cccc(CN2C(=O)Oc3cc(Oc4cccnn4)c(F)cc3[C@H]2C)c1F]

E-82 was synthesized by following similar synthesis sequence and experimental procedures as described for the synthesis of E-65, using key intermediates I-24 and I-31. LCMS (ES) m/z: 490.3 [M−H]⁺. ¹H NMR (400 MHz, DMSO-d₆): δ 9.41 (s, 1H); 9.04 (d, J=3.6 Hz, 1H), 7.84-7.80 (m, 1H); 7.61 (dd, J₁=1.2 Hz, J₂=8.8 Hz, 1H); 7.47 (d, J=10.4 Hz, 1H); 7.42-7.34 (m, 1H); 7.31 (d, J=6.4 Hz, 1H); 7.22-7.04 (m, 3H); 4.96 (d, J=15.6, 1H); 4.61 (q, J=6.4 Hz, 1H); 4.48 (d, J=15.6 Hz, 1H); 2.52 (d, J=4.8 Hz, 3H); 1.46 (d, J=6.4 Hz, 3H). Note: Absolute configuration not determined.

### Example 83: Preparation of (R)-5-fluoro-3-({2-fluoro-3-(methylsulfamoyl)amino]phenyl}methyl)-4-methyl-7-(pyridazin-3-yloxy)-3,4-dihydro-2H-1,3-benzoxazin-2-one or (S)-5-fluoro-3-({2-fluoro-3-(methylsulfamoyl)amino]phenyl}methyl)-4-methyl-7-(pyridazin-3-yloxy)-3,4-dihydro-2H-1,3-benzoxazin-2-one (E-83; Compound 57)

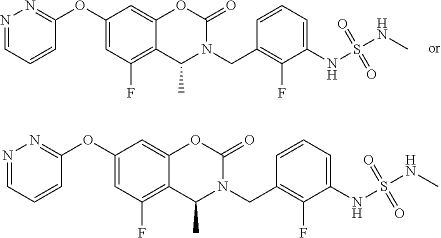 [SMILES: CNS(=O)(=O)Nc1cccc(CN2C(=O)Oc3cc(Oc4cccnn4)cc(F)c3[C@H]2C)c1F ; CNS(=O)(=O)Nc1cccc(CN2C(=O)Oc3cc(Oc4cccnn4)cc(F)c3[C@@H]2C)c1F]

E-83 was synthesized by following similar synthesis sequence and experimental procedures as described for the synthesis of E-65, using key intermediates I-25 and I-31. LCMS (ES) m/z: 492.3 [M+H]⁺. ¹H NMR (400 MHz, DMSO-d₆): δ 9.38 (s, 1H); 9.06 (dd, J₁=1.2 Hz, J₂=4.8 Hz, 1H); 7.83-7.79 (m, 1H); 7.54 (dd, Jᵢ=1.2 Hz, J₂=8.8 Hz, 1H); 7.41-7.34 (m, 1H); 7.20-7.05 (m, 4H); 6.99 (s, 1H); 4.93 (d, J=15.6 Hz, 1H); 4.77 (q, J=6.4 Hz, 1H); 4.53 (d, J=15.6 Hz, 1H); 2.50 (d, J=4.8 Hz, 3H); 1.46 (d, J=6.4 Hz, 3H). Note: Absolute configuration not determined.

### Example 84: Preparation of (S)-5-fluoro-3-({2-fluoro-3-(methylsulfamoyl)amino]phenyl}methyl)-4-methyl-7-(pyridazin-3-yloxy)-3,4-dihydro-2H-1,3-benzoxazin-2-one or (R)-5-fluoro-3-({2-fluoro-3-(methylsulfamoyl)amino]phenyl}methyl)-4-methyl-7-(pyridazin-3-yloxy)-3,4-dihydro-2H-1,3-benzoxazin-2-one (E-84; Compound 56)

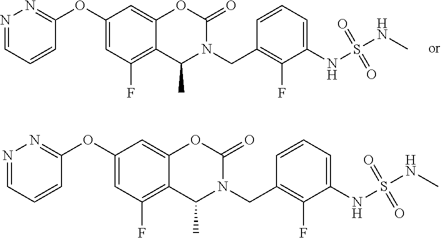 [SMILES: CNS(=O)(=O)Nc1cccc(CN2C(=O)Oc3cc(Oc4cccnn4)cc(F)c3[C@@H]2C)c1F ; CNS(=O)(=O)Nc1cccc(CN2C(=O)Oc3cc(Oc4cccnn4)cc(F)c3[C@H]2C)c1F]

E-84 was synthesized by following similar synthesis sequence and experimental procedures as described for the synthesis of E-65, using key intermediates I-25 and I-31. LCMS (ES) m/z: 492.3 [M+H]⁺. ¹H NMR (400 MHz, DMSO-d₆): (9.41 (s, 1H); 9.06 (dd, J₁=1.2 Hz, J₂=4.8 Hz, 1H); 7.83-7.79 (m, 1H); 7.54 (dd, J₁=1.2 Hz, J₂=8.8 Hz, 1H); 7.41-7.34 (m, 1H); 7.24-7.06 (m, 4H); 6.98 (s, 1H); 4.93 (d, J=15.6 Hz, 1H); 4.77 (q, J=6.4 Hz, 1H); 4.53 (d, J=15.6 Hz, 1H); 2.50 (d, J=4.8 Hz, 3H); 1.46 (d, J=6.4 Hz, 3H). Note: Absolute configuration not determined.

Examples 85 and 86: Preparations of (R)-6-chloro-7-(dimethylaminocarbonyloxy)-3-{[2-fluoro-3-(methylaminosulfonylamino)phenyl]methyl}-4-methyl-3,4-dihydro-2H-1,3-benzoxazin-2-one and (S)-6-chloro-7-(dimethylaminocarbonyloxy)-3-{[2-fluoro-3-(methylaminosulfonylamino)phenyl]methyl}-4-methyl-3,4-dihydro-2H-1,3-benzoxazin-2-one (E-85 and E-86; Compounds 107 and 108, respectively)

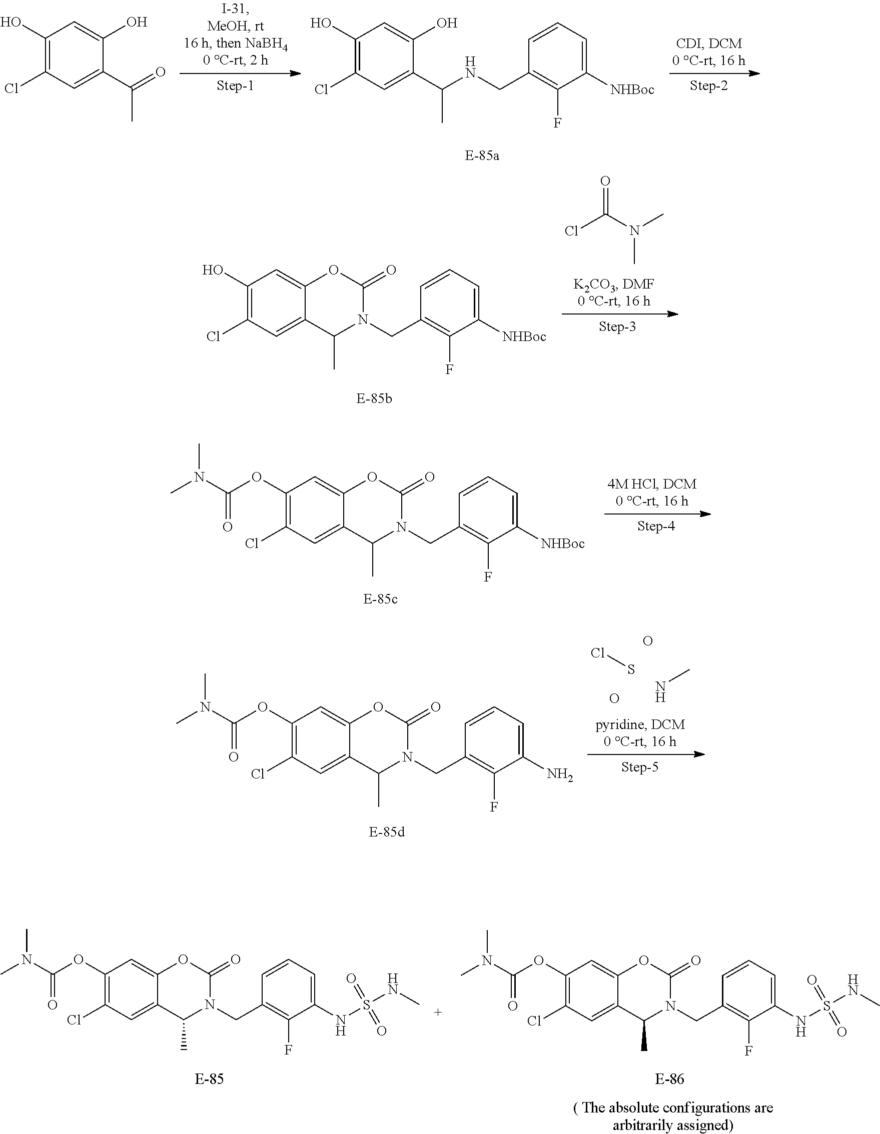

Step-1: Synthesis of tert-butyl N-[3-({[1-(5-chloro-2,4-dihydroxyphenyl)ethyl]amino}methyl)-2-fluorophenyl]carbamate (E-85a): Compound E-85a (0.8 g) was synthesized by following procedure as described for the synthesis of E-01 (step-1) using 1-(5-chloro-2,4-dihydroxyphenyl)ethan-1-one (1.0 g, 5.36 mmol) and tert-butyl N-[3-(aminomethyl)-2-fluorophenyl]carbamate (I-31) (1.29 g, 5.36 mmol) as the starting materials. LCMS (ES) m/z: 411.3 [M+H]⁺.

Step-2: Synthesis of tert-butyl (2-fluoro-3-((8-fluoro-7-hydroxy-4-methyl-2-oxo-2H-benzo[e][1,3]oxazin-3(4H)-yl)methyl)phenyl)carbamate (E-85b): Compound E-85b (0.75 g) was synthesized by following procedure as described for the synthesis of E-01 (step-2) using tert-butyl N-[3-({[1-(5-chloro-2,4-dihydroxyphenyl)ethyl]amino}methyl)-2-fluorophenyl]carbamate (E-85a) (0.80 g, 1.95 mmol) as the starting material. LCMS (ES) m/z: 435.1 [M−H]⁺.

Step-3: Synthesis of 3-[(3-{[(tert-butoxy)carbonyl]amino}-2-fluorophenyl)methyl]-6-chloro-4-methyl-2-oxo-3,4-dihydro-2H-1,3-benzoxazin-7-yl N,N-dimethylcarbamate (E-85c): Compound E-85c (0.48 g) was synthesized by following procedure as described for the synthesis of E-01 (step-4) using tert-butyl (2-fluoro-3-((8-fluoro-7-hydroxy-4-methyl-2-oxo-2H-benzo[e][1,3]oxazin-3(4H)-yl)methyl)phenyl)carbamate (E-85b) (0.750 g, 1.72 mmol) as the starting material. LCMS (ES) m/z: 408.2 [(M-Boc)+H]⁺.

Step-4: Synthesis of 3-[(3-amino-2-fluorophenyl)methyl]-6-chloro-4-methyl-2-oxo-3,4-dihydro-2H-1,3-benzoxazin-7-yl N,N-dimethylcarbamate (E-85d): Compound E-85d (0.38 g) was synthesized by following procedure as described for the synthesis of E-17 (step-3) using 3-[(3-{[(tert-butoxy)carbonyl]amino}-2-fluorophenyl)methyl]-6-chloro-4-methyl-2-oxo-3,4-dihydro-2H-1,3-benzoxazin-7-yl N,N-dimethylcarbamate (E-85c) (0.480 g, 0.945 mmol) as the starting material. LCMS (ES) m/z: 408.2 [M+H]⁺. Example E-85 (0.019 g) and E-86 (0.019 g) were synthesized by following procedure as described for the synthesis of E-17 (step-4) using 3-[(3-amino-2-fluorophenyl)methyl]-6-chloro-4-methyl-2-oxo-3,4-dihydro-2H-1,3-benzoxazin-7-yl N,N-dimethylcarbamate (E-85d) (0.2 g, 0.49 mmol) as the starting material. Enantiomers were separated by chiral preparative HPLC [using CHIRALPAK IG (100 mm×4.6 mm, 3 μm particle size) column, 0.1% DEA in EtOH and n-hexane as mobile phase with 1.0 mL/min flow rate]. Absolute configuration of both enantiomers was not determined.

Step-5: Synthesis of 3-(2-fluoro-3-((N-methylsulfamoyl)amino)benzyl)-2-oxo-6-(trifluoromethyl)-3,4-dihydro-2H-benzo[e][1,3]oxazin-7-yl dimethylcarbamate (E-85): Example E-85 (0.019 g) and E-86 (0.019 g) were synthesized by following procedure as described for the synthesis of E-17 (step-4) using 3-[(3-amino-2-fluorophenyl)methyl]-6-chloro-4-methyl-2-oxo-3,4-dihydro-2H-1,3-benzoxazin-7-yl N,N-dimethylcarbamate (E-85d) (0.2 g, 0.49 mmol) as the starting material. Enantiomers were separated by chiral preparative HPLC [using CHIRALPAK IG (100 mm×4.6 mm, 3 μm particle size) column, 0.1% DEA in EtOH and n-hexane as mobile phase with 1.0 mL/min flow rate]. Absolute configuration of both enantiomers was not determined.

E-85: LCMS (ES) m/z: 501.3 [M+H]⁺. ¹H NMR (400 MHz, DMSO d₆): δ 9.40 (s, 1H); 7.56 (s, 1H); 7.37 (t, J=7.2 Hz, 1H); 7.15-7.04 (m, 4H); 4.92 (d, J=15.6 Hz, 1H); 4.60 (q, J=6.4 Hz, 1H); 4.46 (d, J=15.6 Hz, 1H); 3.06 (s, 3H); 2.92 (s, 3H); 2.52 (d, J=4.4 Hz, 3H); 1.41 (d, J=6.4 Hz, 3H).

E-86: LCMS (ES) m/z: 501.3 [M+H]⁺. ¹H NMR (400 MHz, DMSO d₆): δ 9.40 (s, 1H); 7.56 (s, 1H); 7.37 (t, J=7.2 Hz, 1H); 7.15-7.04 (m, 4H); 4.92 (d, J=15.6 Hz, 1H); 4.60 (q, J=6.4 Hz, 1H); 4.46 (d, J=15.6 Hz, 1H); 3.06 (s, 3H); 2.92 (s, 3H); 2.52 (d, J=4.4 Hz, 3H); 1.41 (d, J=6.4 Hz, 3H).

### Examples 87 and 88: Preparations of (R)-3-{[3-(dimethylaminosulfonylamino)-2-fluorophenyl]methyl}-8-fluoro-4-methyl-7-(3-pyridazinyloxy)-3,4-dihydro-2H-1,3-benzoxazin-2-one and (S)-3-{[3-(dimethylaminosulfonylamino)-2-fluorophenyl]methyl}-8-fluoro-4-methyl-7-(3-pyridazinyloxy)-3,4-dihydro-2H-1,3-benzoxazin-2-one (E-87 and E-88; Compounds 36 and 35, respectively)

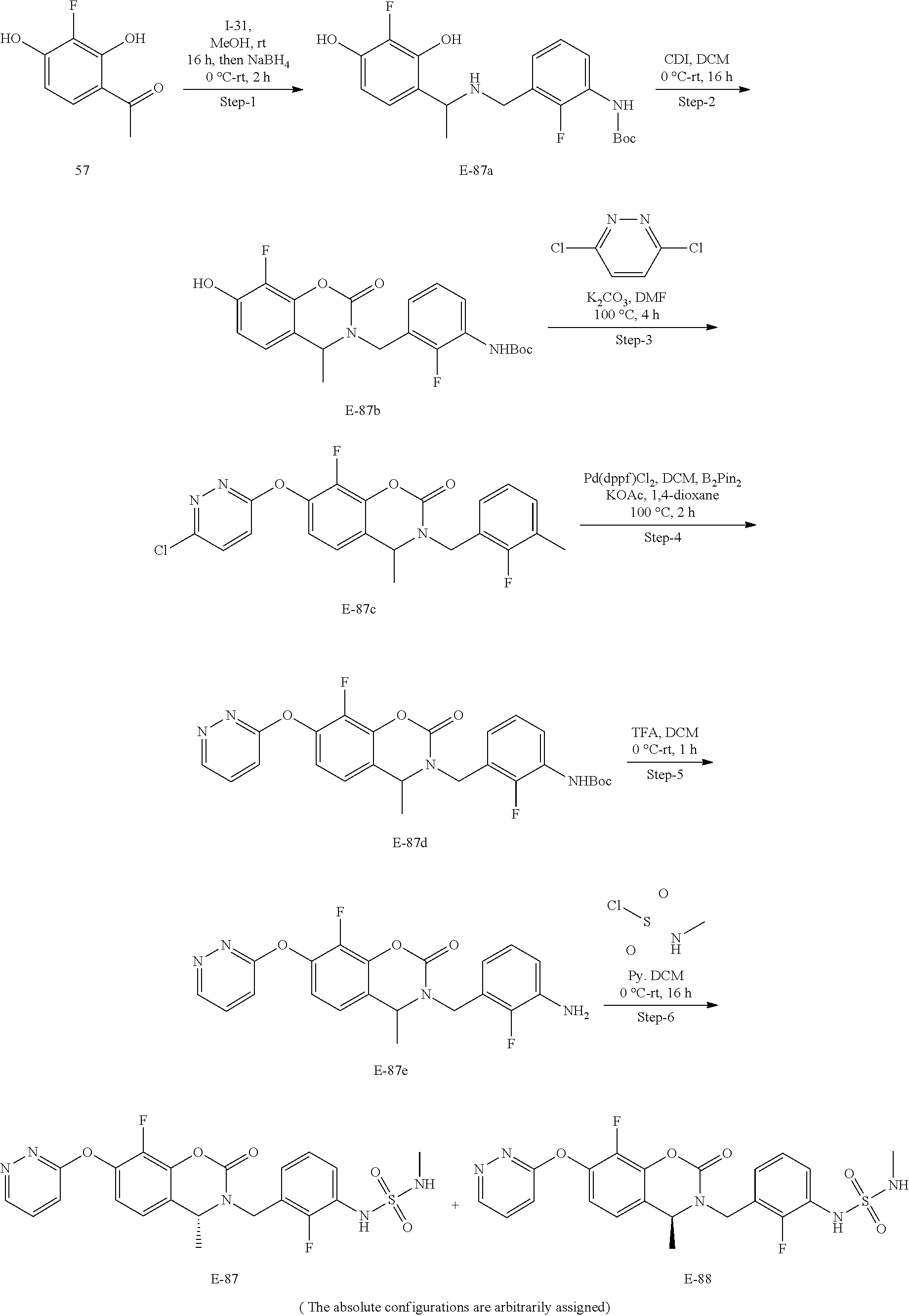

Step-1: Synthesis of tert-butyl (2-fluoro-3-(((1-(3-fluoro-2,4-dihydroxyphenyl)ethyl)amino)methyl)phenyl)carbamate (E-87a): Compound E-87a (4.5 g) was synthesized by following procedure as described for the synthesis of E-01 (step-1) using 1-(3-fluoro-2,4-dihydroxyphenyl)ethan-1-one (2.5 g, 14.7 mmol) and tert-butyl N-[3-(aminomethyl)-2-fluorophenyl]carbamate (I-31) (3.53 g, 14.7 mmol) as the starting materials. LCMS (ES) m/z: 395.3 [M+H]⁺.

Step-2: Synthesis of tert-butyl N-{2-fluoro-3-[(8-fluoro-7-hydroxy-4-methyl-2-oxo-3,4-dihydro-2H-1,3-benzoxazin-3-yl)methyl]phenyl}carbamate (E-87b): Compound E-87b (2.1 g) was synthesized by following procedure as described for the synthesis of E-01 (step-2) using tert-butyl N-[2-fluoro-3-({[1-(3-fluoro-2,4-dihydroxyphenyl)ethyl]amino}methyl)phenyl]carbamate (E-87a) (4.5 g, 7.42 mmol) as the starting material. LCMS (ES) m/z: 419.1 [M−H]⁺.

Step-3: Synthesis of tert-butyl N-[3-({7-[(6-chloropyridazin-3-yl)oxy]-8-fluoro-4-methyl-2-oxo-3,4-dihydro-2H-1,3-benzoxazin-3-yl}methyl)-2-fluorophenyl]carbamate (E-87c): Compound E-87c (0.28 g) was synthesized by following procedure as described for the synthesis of E-32 (step-1) using tert-butyl N-{2-fluoro-3-[(8-fluoro-7-hydroxy-4-methyl-2-oxo-3,4-dihydro-2H-1,3-benzoxazin-3-yl)methyl]phenyl}carbamate (E-87b) (0.55 g, 1.31 mmol) as the starting material. LCMS (ES) m/z: 531.1 [M−H]⁺.

Step-4: Synthesis of tert-butyl N-(2-fluoro-3-{[8-fluoro-4-methyl-2-oxo-7-(pyridazin-3-yloxy)-3,4-dihydro-2H-1,3-benzoxazin-3-yl]methyl}phenyl)carbamate (E-87d): Compound E-87d (1.2 g) was synthesized by following procedure as described for the synthesis of E-32 (step-2) using tert-butyl N-[3-({7-[(6-chloropyridazin-3-yl)oxy]-8-fluoro-4-methyl-2-oxo-3,4-dihydro-2H-1,3-benzoxazin-3-yl}methyl)-2-fluorophenyl]carbamate (E-87c) (1.3 g, 2.44 mol). LCMS (ES) m/z: 499.1 [M+H]⁺.

Step-5: Synthesis of 3-[(3-amino-2-fluorophenyl)methyl]-8-fluoro-4-methyl-7-(pyridazin-3-yloxy)-3,4-dihydro-2H-1,3-benzoxazin-2-one (E-87e): Compound E-87e (0.55 g) was synthesized by following procedure as described for the synthesis of E-43 (step-4) using tert-butyl N-(2-fluoro-3-{[8-fluoro-4-methyl-2-oxo-7-(pyridazin-3-yloxy)-3,4-dihydro-2H-1,3-benzoxazin-3-yl]methyl}phenyl)carbamate (E-87d) (0.7 g, 1.4 mmol) as the starting material. LCMS (ES) m/z: 399.1 [M+H]⁺.

Step-6: Synthesis of 8-fluoro-3-(12-fluoro-3-(methylsulfamoyl)amino]phenyl}methyl)-4-methyl-7-(pyridazin-3-yloxy)-3,4-dihydro-2H-1,3-benzoxazin-2-one (E-87): Example E-87 (0.020 g) and E-88 (0.028 g) were synthesized by following procedure as described for the synthesis of E-01 (step-6) using 3-[(3-amino-2-fluorophenyl)methyl]-8-fluoro-4-methyl-7-(pyridazin-3-yloxy)-3,4-dihydro-2H-1,3-benzoxazin-2-one (E-87e) (0.5 g, 1.26 mmol) as the starting material. Both enantiomers were separated by chiral preparative HPLC [using CHIRALPAK IG (100 mm×4.6 mm, 3 μm particle size) column, 0.1% DEA in methanol as a mobile phase with 0.5 mL/min flow rate]. Note: Absolute configuration of both enantiomers was not determined.

Example 87: LCMS (ES) m/z: 492.3 [M+H]⁺. ¹H NMR (400 MHz, DMSO-d₆): δ 9.41 (s, 1H); 9.05 (d, J=4.4 Hz, 1H); 7.85-7.81 (m, 1H); 7.63 (d, J=8.8 Hz, 1H); 7.40-7.36 (m, 1H); 7.26-7.12 (m, 5H); 4.96 (d, J=15.6 Hz, 1H); 4.70 (d, J=6.4 Hz, 1H); 4.52 (d, J=15.6 Hz, 1H); 2.50 (d, J=4.8 Hz, 3H); 1.46 (d, J=6.4 Hz, 3H).

Example 88: LCMS (ES) m/z: 492.3 [M+H]⁺. ¹H NMR (400 MHz, DMSO-d₆): δ 9.41 (s, 1H); 9.05 (dd, J₁=1.2 Hz, J₂=4.4 Hz, 1H); 7.85-7.81 (m, 1H); 7.63 (dd, J₁=1.2 Hz, J₂=8.8 Hz, 1H); 7.42-7.36 (m, 1H); 7.26-7.10 (m, 5H); 4.96 (d, J=15.6 Hz, 1H); 4.70 (d, J=6.4 Hz, 1H); 4.52 (d, J=15.6 Hz, 1H); 2.50 (d, J=4.8 Hz, 3H); 1.46 (d, J=6.4 Hz, 3H).

### Examples 89 and 90: Preparation of (R)-3-{[3-(dimethylaminosulfonylamino)-2-fluorophenyl]methyl}-8-fluoro-4-methyl-7-(3-pyridazinyloxy)-3,4-dihydro-2H-1,3-benzoxazin-2-one and (S)-3-{[3-(dimethylaminosulfonylamino)-2-fluorophenyl]methyl}-8-fluoro-4-methyl-7-(3-pyridazinyloxy)-3,4-dihydro-2H-1,3-benzoxazin-2-one (E-89 and E-90; Compounds 43 and 44, respectively)

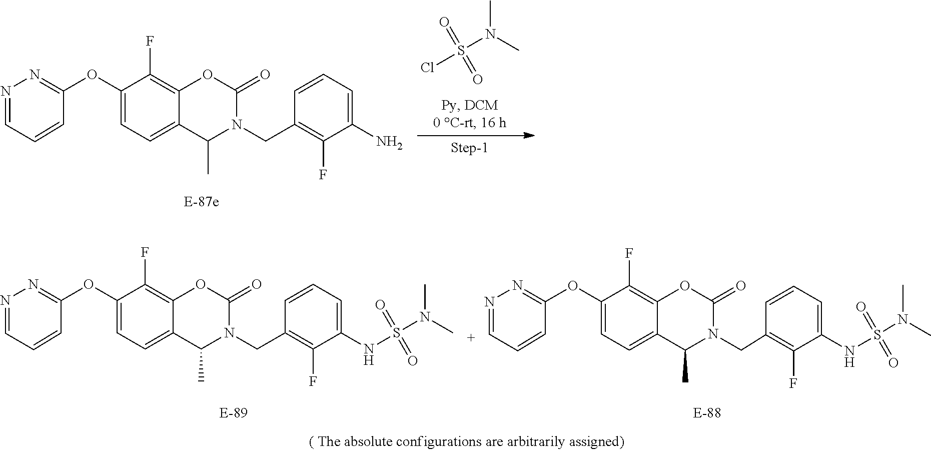

Step-1: Synthesis of 3-{[3-(dimethylaminosulfonylamino)-2-fluorophenyl]methyl}-8-fluoro-4-methyl-7-(3-pyridazinyloxy)-3,4-dihydro-2H-1,3-benzoxazin-2-one (E-89): Example E-89 (0.030 g) and E-90 (0.030 g) were synthesized by following procedure as described for the synthesis of E-01 (step-6) using 3-(3-amino-2-fluorobenzyl)-8-fluoro-4-methyl-7-(pyridazin-3-yloxy)-3,4-dihydro-2H-benzo[e][1,3]oxazin-2-one (E-87e) (200 mg, 502 μmol) as the starting material. Both the enantiomers were separated by chiral HPLC [using CHIRALPAK IC (100 mm×4.6 mm, 5 μm particle size) column, n-Hexane:Ethanol (40:60) as the mobile phase with 1 mL/min flow rate]. Absolute configuration of enantiomers was not determined.

Example 89: LCMS (ES) m/z: 506.3 [M+H]⁺. ¹H NMR (400 MHz, DMSO-d₆) δ 9.61 (s, 1H); 9.05 (dd, J₁=1.2 Hz, J₂=4.4 Hz, 1H); 7.85-7.81 (m, 1H); 7.63 (dd, J₁=1.2 Hz, J₂=8.8 Hz, 1H); 7.41-7.37 (m, 1H); 7.24-7.14 (m, 4H); 4.99 (d, J=15.6 Hz, 1H); 4.68 (q, J=6.4 Hz, 1H); 4.52 (d, J=15.6 Hz, 1H); 2.63 (s, 6H); 1.48 (d, J=6.4 Hz, 3H).

Example 90: LCMS (ES) m/z: 506.3 [M+H]⁺. ¹H NMR (400 MHz, DMSO-d₆) δ 9.61 (s, 1H); 9.05 (dd, J₁=1.2 Hz, J₂=4.4 Hz, 1H); 7.85-7.81 (m, 1H); 7.63 (dd, J₁=1.2 Hz, J₂=8.8 Hz, 1H); 7.41-7.37 (m, 1H); 7.24-7.12 (m, 4H); 4.99 (d, J=15.6 Hz, 1H); 4.68 (q, J=6.4 Hz, 1H); 4.52 (d, J=15.6 Hz, 1H); 2.63 (s, 6H); 1.48 (d, J=6.4 Hz, 3H).

### Example 91: Preparation of (S)-N-(2-fluoro-3-((5-fluoro-4-methyl-2-oxo-7-(pyridazin-3-yloxy)-2H-benzo[e][1,3]oxazin-3(4H)-yl)methyl)phenyl)cyclopropanesulfonamide or (R)-N-(2-fluoro-3-((5-fluoro-4-methyl-2-oxo-7-(pyridazin-3-yloxy)-2H-benzo[e][1,3]oxazin-3(4H)-yl)methyl)phenyl)cyclopropanesulfonamide (E-91; Compound 55)

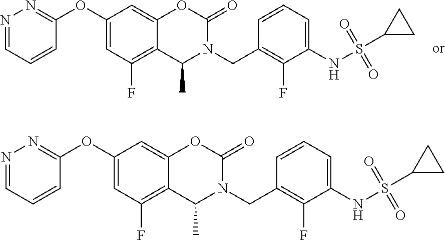 [SMILES: C[C@H]1c2c(F)cc(Oc3cccnn3)cc2OC(=O)N1Cc1cccc(NS(=O)(=O)C2CC2)c1F ; C[C@@H]1c2c(F)cc(Oc3cccnn3)cc2OC(=O)N1Cc1cccc(NS(=O)(=O)C2CC2)c1F]

E-91 was synthesized by following similar synthesis sequence and experimental procedures as described for the synthesis of E-89, using key intermediates I-23 and I-31. (note: cyclopropylsulfonyl chloride was used in final step). LCMS (ES) m/z: 503.4 [M+H]⁺.

¹H NMR (400 MHz, DMSO-d₆): δ 9.63 (s, 1H); 9.06-9.04 (m, 1H); 7.83-7.79 (m, 1H); 7.54 (dd, J₁=1.2 Hz, J₂=8.8 Hz, 1H); 7.40-7.32 (m, 1H); 7.24-7.10 (m, 3H); 6.99 (s, 1H); 4.96 (d, J=15.6 Hz, 1H); 4.76 (q, J=6.4 Hz, 1H); 4.56 (d, J=15.6 Hz, 1H); 2.62-2.56 (m, 1H); 1.46 (d, J=6.4 Hz, 3H); 0.92-0.80 (m, 4H). Note: Absolute configuration not determined.

### Example 92: Preparation of (R)-N-(2-fluoro-3-((5-fluoro-4-methyl-2-oxo-7-(pyridazin-3-yloxy)-2H-benzo[e][1,3]oxazin-3(4H)-yl)methyl)phenyl)cyclopropanesulfonamide or (S)-N-(2-fluoro-3-((5-fluoro-4-methyl-2-oxo-7-(pyridazin-3-yloxy)-2H-benzo[e][1,3]oxazin-3(4H)-yl)methyl)phenyl)cyclopropanesulfonamide (E-92; Compound 54)

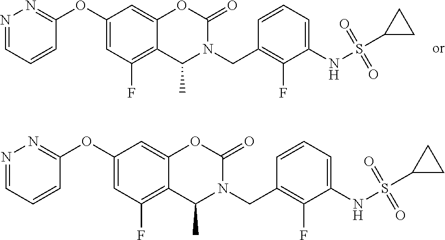 [SMILES: C[C@@H]1c2c(F)cc(Oc3cccnn3)cc2OC(=O)N1Cc1cccc(NS(=O)(=O)C2CC2)c1F ; C[C@H]1c2c(F)cc(Oc3cccnn3)cc2OC(=O)N1Cc1cccc(NS(=O)(=O)C2CC2)c1F]

E-92 was synthesized by following similar synthesis sequence and experimental procedures as described for the synthesis of E-89, using key intermediates I-23 and I-31. (note: cyclopropylsulfonyl chloride was used in final step). LCMS (ES) m/z: 503.3 [M+H]⁺.

¹H NMR (400 MHz, DMSO-d₆): δ 9.63 (s, 1H); 9.06-9.04 (m, 1H); 7.83-7.79 (m, 1H); 7.54 (dd, J₁=1.2 Hz, J, =8.8 Hz, 1H); 7.40-7.32 (m, 1H); 7.24-7.10 (m, 3H); 6.99 (s, 1H); 4.96 (d, J=15.6 Hz, 1H); 4.76 (q, J=6.4 Hz, 1H); 4.56 (d, J=15.6 Hz, 1H); 2.62-2.56 (m, 1H); 1.46 (d, J=6.4 Hz, 3H); 0.92-0.80 (m, 4H). Note: Absolute configuration not determined.

### Example 93: Preparation of 3-({2-fluoro-3-[(methylsulfamoyl)amino]phenyl}methyl)-4,4-dimethyl-7-(1,3-oxazol-2-yloxy)-3,4-dihydro-2H-1,3-benzoxazin-2-one (E-93; Compound 74)

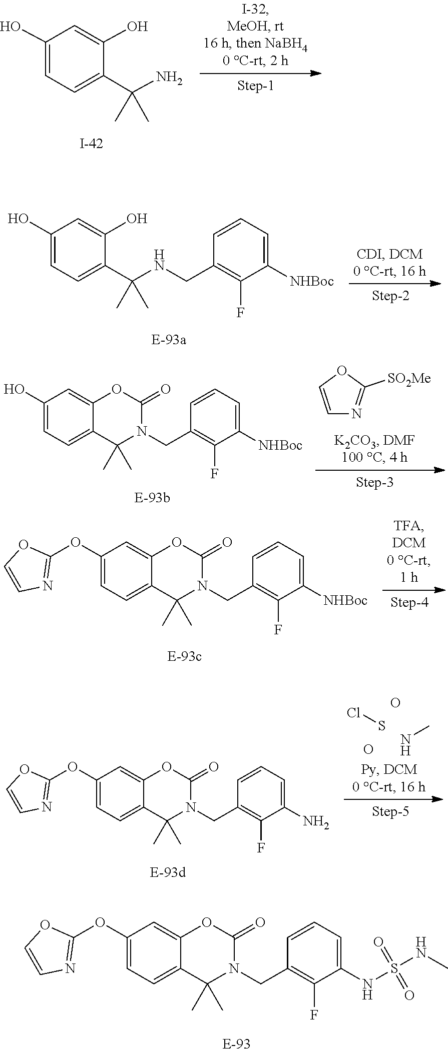

Step-1: Synthesis of tert-butyl N-[3-({[2-(2,4-dihydroxyphenyl)propan-2-yl]amino}methyl)-2-fluorophenyl]carbamate (E-93a): Compound E-93a (0.24 g) was synthesized by following procedure as described for the synthesis of E-01 (step-1) using of 4-(2-aminopropan-2-yl)benzene-1,3-diol (I-42) (0.37 g, 1.55 mmol) and tert-butyl N-(2-fluoro-3-formylphenyl)carbamate (1-32) (0.371 g, 1.55 mmol) as the starting materials. LCMS (ES) m/z: 391.14 [M+H]⁺.

Step-2: Synthesis of tert-butyl N-{2-fluoro-3-[(7-hydroxy-4,4-dimethyl-2-oxo-3,4-dihydro-2H-1,3-benzoxazin-3-yl)methyl]phenyl}carbamate (E-93b): Compound E-93b (0.15 g) was synthesized by following procedure as described for the synthesis of E-01 (step-2) using tert-butyl N-[3-({[2-(2,4-dihydroxyphenyl)propan-2-yl]amino}methyl)-2-fluorophenyl]carbamate (E-93a) (0.24 g, 0.615 mmol) as the starting material. LCMS (ES) m/z: 415.28 [M−H]⁺.

Step-3: Synthesis of tert-butyl N-(3-{[4,4-dimethyl-7-(1,3-oxazol-2-yloxy)-2-oxo-3,4-dihydro-2H-1,3-benzoxazin-3-yl]methyl}-2-fluorophenyl)carbamate (E-93c): To a stirred solution of tert-butyl N-{2-fluoro-3-[(7-hydroxy-4,4-dimethyl-2-oxo-3,4-dihydro-2H-1,3-benzoxazin-3-yl)methyl]phenyl}carbamate (E-93b) (0.14 g, 0.336 mmol) in DMF (15 mL) was added K₂CO₃ (0.929 g, 0.672 mmol) and 2-methanesulfonyl-1,3-oxazole (0.594 g, 0.403 mmol) at room temperature. The reaction mixture was allowed to stir at 100° C., while monitoring reaction progress by TLC. After completion of the reaction (4 h), the reaction mixture was filtered and washed with EtOAc. The filtrate was concentrated under reduced pressure and the residue was purified by Combi-Flash (using gradient elution of 0-50% of EtOAc in heptane) to afford the desired E-93c (0.045 g). LCMS (ES) m/z: 482.08 [M−H]⁺.

Step-4: Synthesis of 3-[(3-amino-2-fluorophenyl)methyl]-4,4-dimethyl-7-(1,3-oxazol-2-yloxy)-3,4-dihydro-2H-1,3-benzoxazin-2-one (E-93d): Compound E-93d (0.025 g) was synthesized by following procedure as described for the synthesis of E-43 (step-4) using tert-butyl N-(3-{[4,4-dimethyl-7-(1,3-oxazol-2-yloxy)-2-oxo-3,4-dihydro-2H-1,3-benzoxazin-3-yl]methyl}-2-fluorophenyl)carbamate (E-93c) (0.045 g, 0.093 mmol) as the starting material. LCMS (ES) m/z: 384.16 [M+H]⁺.

Step-5: Synthesis of 3-({2-fluoro-3-[(methylsulfamoyl)amino]phenyl}methyl)-4,4-dimethyl-7-(1,3-oxazol-2-yloxy)-3,4-dihydro-2H-1,3-benzoxazin-2-one (E-93): Example 93 (0.019 g) was synthesized by following procedure as described for the synthesis of E-17 (step-4) using 3-[(3-amino-2-fluorophenyl)methyl]-4,4-dimethyl-7-(1,3-oxazol-2-yloxy)-3,4-dihydro-2H-1,3-benzoxazin-2-one (E-93d) (0.075 g, 0.196 mmol) as the starting material. The final compound was purified by reverse phase preparative HPLC purification [using X Select Phenyl Hexyl (19 mm×250 mm, 5 μm particle size) column, 5% ammonia in water and acetonitrile as mobile phase with 17 mL/min flow rate]. LCMS (ES) m/z: 477.20 [M+H]⁺. ¹H NMR (400 MHz, DMSO-d₆): δ 8.22 (br s, 1H); 7.85 (d, J=0.8 Hz, 1H); 7.57 (d, J=9.6 Hz, 1H); 7.35-7.28 (m, 1H); 7.26-7.20 (m, 2H); 7.08-7.02 (m, 2H); 6.95 (t, J=6.8 Hz, 1H); 4.77 (s, 2H); 2.52 (d, J=4.4 Hz, 3H); 1.58 (s, 6H).

### Example 94: Preparation of 3-({2-fluoro-3-[(methylsulfamoyl)amino]phenyl}methyl)-4,4-dimethyl-7-(pyridazine-3-yloxy)-3,4-dihydro-2H-1,3-benzoxazin-2-one (E-94; Compound 51)

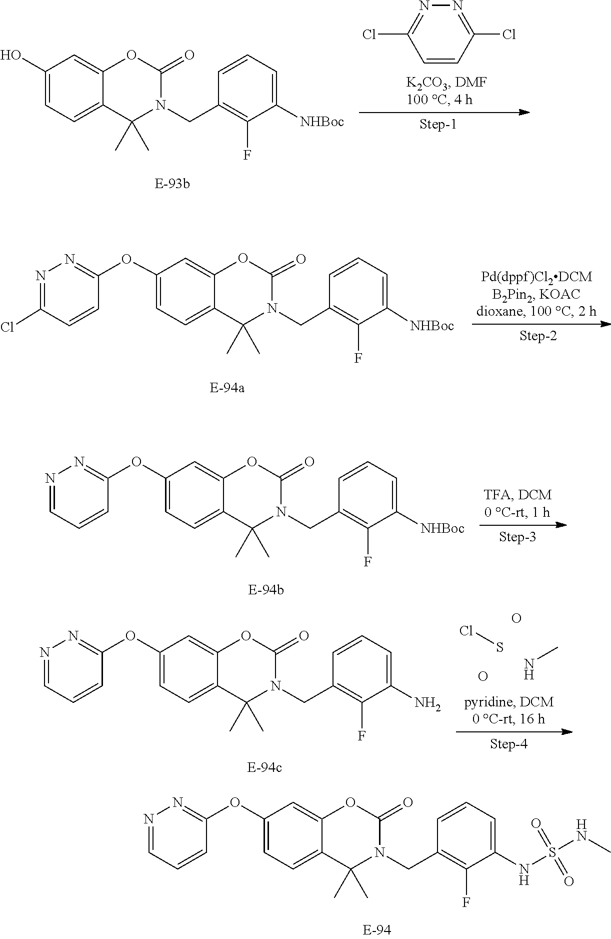

Step-1: Synthesis of tert-butyl N-[3-({7-[(6-chloropyridazin-3-yl)oxy]-4,4-dimethyl-2-oxo-3,4-dihydro-2H-1,3-benzoxazin-3-yl}methyl)-2-fluorophenyl]carbamate (E-94a): Compound E-94a (0.38 g) was synthesized by following procedure as described for the synthesis of E-32 (step-1) using tert-butyl N-{2-fluoro-3-[(7-hydroxy-4,4-dimethyl-2-oxo-3,4-dihydro-2H-1,3-benzoxazin-3-yl)methyl]phenyl}carbamate (E-93b) (0.360 g, 0.864 mmol) as the starting material. ¹H NMR (400 MHz, DMSO-d₆): δ 8.98 (s, 1H); 7.99 (d, J=9.2 Hz, 1H); 7.65 (d, J=9.2 Hz, 1H); 7.57 (d, J=8.4 Hz, 1H); 7.47-7.44 (m, 1H); 7.12-7.09 (m, 2H); 7.05 (t, J=8.0 Hz, 1H); 6.95-6.92 (m, 1H); 4.77 (s, 2H); 1.59 (s, 6H); 1.46 (s, 9H).

Step-2: Synthesis of tert-butyl N-(3-{[4,4-dimethyl-2-oxo-7-(pyridazine-3-yloxy)-3,4-dihydro-2H-1,3-benzoxazin-3-yl]methyl}-2-fluorophenyl)carbamate (E-94b): Compound E-94b (0.14 g) was synthesized by following procedure as described for the synthesis of E-32 (step-2) using tert-butyl N-[3-({7-[(6-chloropyridazin-3-yl)oxy]-4,4-dimethyl-2-oxo-3,4-dihydro-2H-1,3-benzoxazin-3-yl}methyl)-2-fluorophenyl]carbamate (E-94a) (0.38 g, 0.718 mmol) as the starting material. LCMS (ES) m/z: 495.38 [M+H]⁺.

Step-3: Synthesis of 3-[(3-amino-2-fluorophenyl)methyl]-4,4-dimethyl-7-(pyridazine-3-yloxy)-3,4-dihydro-2H-1,3-benzoxazin-2-one (E-94c): Compound E-94c (0.11 g) was synthesized by following procedure as described for the synthesis of E-43 (step-4) using tert-butyl N-(3-{[4,4-dimethyl-2-oxo-7-(pyridazine-3-yloxy)-3,4-dihydro-2H-1,3-benzoxazin-3-yl]methyl}-2-fluorophenyl)carbamate (E-94b) (0.142 g, 0.287 mmol) as the starting material. LCMS (ES) m/z: 395.17 [M+H]⁺.

Step-4: Synthesis of 3-({2-fluoro-3-[(methylsulfamoyl)amino]phenyl}methyl)-4,4-dimethyl-7-(pyridazine-3-yloxy)-3,4-dihydro-2H-1,3-benzoxazin-2-one (E-94): Example E-94 (0.022 g) was synthesized by following procedure as described for the synthesis of E-01 (step-6) using 3-[(3-amino-2-fluorophenyl)methyl]-4,4-dimethyl-7-(pyridazine-3-yloxy)-3,4-dihydro-2H-1,3-benzoxazin-2-one (E-94c) (0.11 g, 0.279 mmol) as the starting material. The final compound was purified by reverse phase preparative HPLC purification [using X Select Phenyl Hexyl (19 mm×250 mm, 5 m particle size) column, 5% ammonia in water and acetonitrile as mobile phase with 3 mL/min flow rate]. LCMS (ES) m/z: 488.25 [M+H]⁺. ¹H NMR (400 MHz, DMSO-d₆): b 9.42 (br s, 1H); 9.04 (dd, J₁=1.2 Hz, J₂=4.4 Hz, 1H); 7.82-7.78 (m, 1H); 7.58-7.50 (m, 2H); 7.34-7.20 (m, 2H); 7.10-7.05 (m, 3H); 6.98 (t, J=6.8 Hz, 1H); 4.78 (s, 2H); 2.53 (d, J=4.8 Hz, 3H); 1.61 (s, 6H).

### Example 95: Preparation of 3-{[3-(dimethylaminosulfonylamino)-2-fluorophenyl]methyl}-4,4-dimethyl-7-(3-pyridazinyloxy)-3,4-dihydro-2H-1,3-benzoxazin-2-one (E-95; Compound 34)

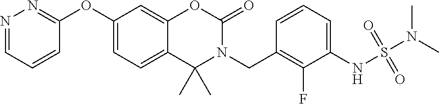 [SMILES: CN(C)S(=O)(=O)Nc1cccc(CN2C(=O)Oc3cc(Oc4cccnn4)ccc3C2(C)C)c1F]

E-95 was synthesized by following similar synthesis sequence and experimental procedures as described for the synthesis of E-94, using key intermediate E-94c and dimethylsulfamoyl chloride as the starting materials. LCMS (ES) m/z: 501.9 [M+H]⁺. ¹H NMR (400 MHz, DMSO-d₆): δ 9.65 (s, 1H); 9.04 (d, J=4.4 Hz, 1H); 7.83-7.79 (m, 1H); 7.58-7.50 (m, 2H); 7.33-7.29 (m, 1H); 7.10-6.98 (m, 4H); 4.79 (s, 2H); 2.69 (s, 6H); 1.61 (s, 6H).

### Example 96: Preparation of 3-({3-fluoro-2-[(methylsulfamoyl)amino]pyridin-4-yl}methyl)-4,4-dimethyl-7-(pyridazin-3-yloxy)-3,4-dihydro-2H-1,3-benzoxazin-2-one (E-96; Compound 32)

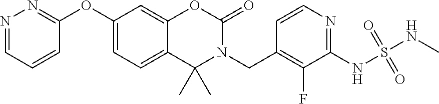 [SMILES: CNS(=O)(=O)Nc1nccc(CN2C(=O)Oc3cc(Oc4cccnn4)ccc3C2(C)C)c1F]

E-96 was synthesized by following similar synthesis sequence and experimental procedures as described for the synthesis of E-93, using key intermediates I-42 and I-35. LCMS (ES) m/z: 489.2 [M+H]⁺. ¹H NMR (400 MHz, DMSO-d₆): δ 10.42 (s, 1H); 9.04 (dd, J/=1.2 Hz, J₂=4.8 Hz, 1H); 7.98 (d, J=4.8 Hz, 1H); 7.82-7.79 (m, 1H); 7.59 (d, J=8.8 Hz, 1H); 7.53 (dd, J₁=1.2 Hz, J₂=8.0 Hz, 1H); 7.09 (dd, J₁=2.4 Hz, J₂=8.4 Hz, 1H); 7.06-7.00 (m, 2H); 6.96-6.92 (m, 1H); 4.81 (s, 2H); 2.52 (d, J=4.4 Hz, 3H); 1.64 (s, 6H).

### Example 97: Preparation of 3-(2-fluoro-3-((N-methylsulfamoyl)amino)benzyl)-2-oxo-2,3-dihydrospiro[benzo[e][1,3]oxazine-4,3′-oxetan]-7-yldimethylcarbamate (E-97; Compound 115)

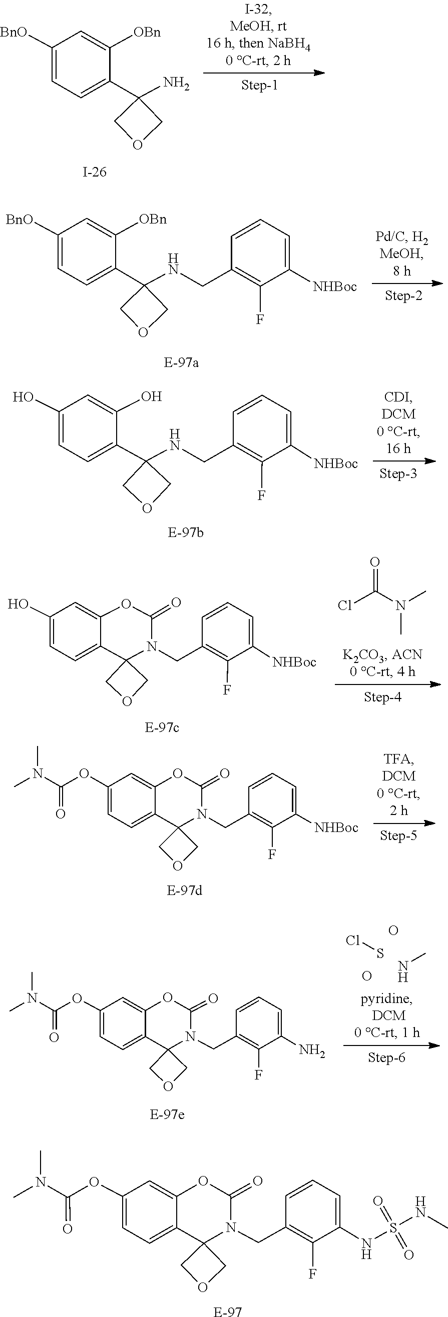

Step-1: Synthesis of tert-butyl N-{3-[({3-[2,4-bis(benzyloxy)phenyl]oxetan-3-yl}amino)methyl]-2-fluorophenyl}carbamate (E-97a): Compound E-97a (0.9 g) was synthesized by following procedure as described for the synthesis of E-01 (step-1) using 3-[2,4-bis(benzyloxy)phenyl]oxetan-3-amine (I-26) (0.7 g, 1.94 mmol) and tert-butyl N-(2-fluoro-3-formylphenyl)carbamate (I-32) (371 mg, 1.55 mmol) as the starting materials. LCMS (ES) m/z: 583.2 [M−H]⁺.

Step-2: Synthesis of tert-butyl N-[3-({[3-(2,4-dihydroxyphenyl)oxetan-3-yl]amino}methyl)-2-fluorophenyl]carbamate (E-97b): Compound E-97b (0.5 g) was synthesized by following procedure as described for the synthesis of E-60 (step-2) using tert-butyl N-{3-[({3-[2,4-bis(benzyloxy)phenyl]oxetan-3-yl}amino)methyl]-2-fluorophenyl}carbamate (E-97a) (0.9 g, 1.54 mmol) as the starting materials. LCMS (ES) m/z: 405.2 [M+H]⁺.

Step-3: Synthesis of tert-butyl N-[2-fluoro-3-({7-hydroxy-2-oxo-2,3-dihydrospiro[1,3-benzoxazine-4,3′-oxetan]-3-yl}methyl)phenyl]carbamate (E-97c): Compound E-97c (0.45 g) was synthesized by following procedure as described for the synthesis of E-01 (step-2) using tert-butyl N-[3-({[3-(2,4-dihydroxyphenyl)oxetan-3-yl]amino}methyl)-2-fluorophenyl]carbamate (E-97b) (0.5 g, 1.24 mmol) as the starting material. LCMS (ES) m/z: 375.1 [(M-^{t}Bu)+H]⁺.

Step-4: Synthesis of 3-[(3-{[(tert-butoxy)carbonyl]amino}-2-fluorophenyl)methyl]-2-oxo-2,3-dihydrospiro[1,3-benzoxazine-4,3′-oxetan]-7-yl N,N-dimethylcarbamate (E-97d): Compound E-97d (0.3 g) was synthesized by following procedure as described for the synthesis of E-01 (step-4) using tert-butyl N-[2-fluoro-3-({7-hydroxy-2-oxo-2,3-dihydrospiro[1,3-benzoxazine-4,3′-oxetan]-3-yl}methyl)phenyl]carbamate (E-97c) (450 mg, 1.05 mmol) as the starting material. LCMS (ES) m/z: 446.3 [(M-^{t}Bu)+H]⁺.

Step-5: Synthesis of 3-[(3-amino-2-fluorophenyl)methyl]-2-oxo-2,3-dihydrospiro[1,3-benzoxazine-4,3′-oxetan]-7-yl N,N-dimethylcarbamate (E-97e): Compound E-97e (0.250 g) was synthesized by following procedure as described for the synthesis of E-43 (step-4) using 3-[(3-{[(tert-butoxy)carbonyl]amino}-2-fluorophenyl)methyl]-2-oxo-2,3-dihydrospiro[1,3-benzoxazine-4,3′-oxetan]-7-yl N,N-dimethylcarbamate (E-97d) (0.3 g, 598 μmol) as the starting material. LCMS (ES) m/z: 402.2 [M+H]⁺.

Step-6: Synthesis of 3-({2-fluoro-3-[(methylsulfamoyl)amino]phenyl}methyl)-2-oxo-2,3-dihydrospiro[1,3-benzoxazine-4,3′-oxetan]-7-yl N,N-dimethylcarbamate (E-97): Example E-97 (0.058 g) was synthesized by following procedure as described for the synthesis of E-01 (step-6) using 3-[(3-amino-2-fluorophenyl)methyl]-2-oxo-2,3-dihydrospiro[1,3-benzoxazine-4,3′-oxetan]-7-yl N,N-dimethylcarbamate (E-97e) (250 mg, 623 μmol) as the starting material. The final compound was purified by preparative HPLC [using CHIRALPAK IC (100 mm×4.6 mm, 3 μm particle size) column, methanol as mobile phase with 0.5 mL/min flow rate]. LCMS m/z: 495.3 [M+H]⁺. ¹H NMR (400 MHz, DMSO-d₆): δ 9.44 (s, 1H); 7.91 (d, J=8.8 Hz, 1H); 7.34 (t, J=7.6 Hz, 1H); 7.24 (q, J=4.8 Hz, 1H); 7.13 (dd, Jᵢ=2.4 Hz, J₂=8.4 Hz, 1H); 7.07 (t, J=8.0 Hz, 1H); 6.95-6.90 (m, 2H); 5.09 (s, 2H); 5.05 (d, J=8.0 Hz, 2H); 4.77 (d, J=7.6 Hz, 2H); 3.05 (s, 3H); 2.93 (s, 3H); 2.55 (d, J=4.8 Hz, 3H).

### Example 98: Preparation of 6-fluoro-3-({2-fluoro-3-(methylsulfamoyl)amino]phenyl}methyl)-2-oxo-2,3-dihydrospiro[1,3-benzoxazine-4,3′-oxetan]-7-yl N,N-dimethylcarbamate (E-98; Compound 76)

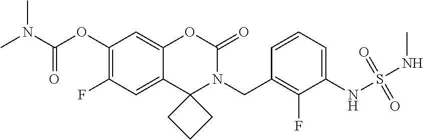

E-98 was synthesized by following similar synthesis sequence and experimental procedures as described for the synthesis of E-97, using key intermediates I-28 and I-32. LCMS (ES) m/z: 511.1 [M−H]⁺. ¹H NMR (400 MHz, DMSO-d₆): δ 9.46 (s, 1H); 7.89 (d, J=6.8 Hz, 1H); 7.36-7.31 (m, 1H); 7.25 (q, J=4.4 Hz, 1H); 7.15 (d, J=6.8 Hz, 1H); 7.07 (t, J=8.0 Hz, 1H); 6.98-6.92 (m, 1H); 5.08 (s, 2H); 5.02 (d, J=8.0 Hz, 2H); 4.80 (d, J=8.0 Hz, 2H); 3.07 (s, 3H); 2.94 (s, 3H); 2.54 (d, J=4.8 Hz, 3H).

### Example 99: Preparation of 8-fluoro-3-({2-fluoro-3-(methylsulfamoyl)amino]phenyl}methyl)-2-oxo-2,3-dihydrospiro[1,3-benzoxazine-4,3′-oxetan]-7-yl N,N-dimethyl carbamate (E-99; Compound 77)

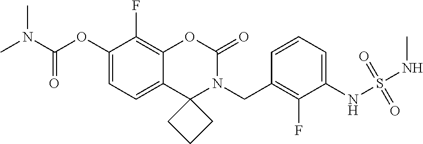

E-99 was synthesized by following similar synthesis sequence and experimental procedures as described for the synthesis of E-97, using key intermediates I-27 and I-32. LCMS (ES) m/z: 513.3 [M+H]⁺. ¹H NMR (400 MHz, DMSO-d₆): δ 9.43 (s, 1H); 7.72 (dd, J₁=1.6 Hz, J₂=8.8 Hz, 1H); 7.33 (t, J=8.0 Hz, 1H); 7.25 (t, J=7.2 Hz, 1H); 7.16-6.90 (m, 3H); 5.08 (s, 4H); 5.06 (d, J=8.0 Hz, 2H); 4.76 (d, J=8.0 Hz, 2H); 3.04 (s, 3H); 2.94 (s, 3H); 2.54 (d, J=4.8 Hz, 3H).

### Example 100: Preparation of 3-({2-fluoro-3-[(methylsulfamoyl)amino]phenyl}methyl)-7-(1,3-oxazol-2-yloxy)-2,3-dihydrospiro[1,3-benzoxazine-4,3′-oxetan]-2-one (E-100; Compound 95)

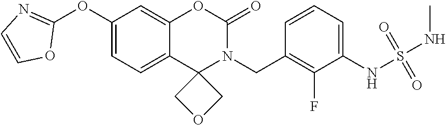 [SMILES: CNS(=O)(=O)Nc1cccc(CN2C(=O)Oc3cc(Oc4ncco4)ccc3C23COC3)c1F]

E-100 was synthesized by following synthesis sequence and procedures as described for the synthesis of E-97, using key intermediate E-97c and 2-(methylsulfonyl)oxazole as the starting materials. LCMS (ES) m/z: 491.3 [M+H]⁺. ¹H NMR (400 MHz, DMSO-d₆): δ 9.44 (s, 1H); 8.00 (d, J=8.8 Hz, 1H); 7.86 (d, J=1.2 Hz, 1H); 7.38 (dd, J₁=2.4 Hz, J₂=8.8 Hz, 1H); 7.36-7.32 (m, 1H); 7.24 (d, J=2.4, Hz, 2H); 7.08-7.04 (m, 2H); 6.98-6.90 (m, 1H); 5.09 (s, 2H); 5.07 (d, J=8.0 Hz, 2H); 4.79 (d, J=8.0 Hz, 2H); 2.55 (d, J=5.2 Hz, 3H).

### Example 101: Preparation of 3-({3-fluoro-2-[(methylsulfamoyl)amino]pyridin-4-yl}methyl)-7-(1,3-oxazol-2-yloxy)-2,3-dihydrospiro[1,3-benzoxazine-4,3′-oxetan]-2-one (E-101; Compound 68)

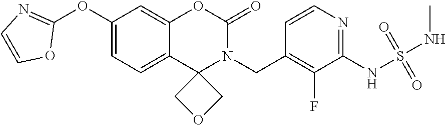 [SMILES: CNS(=O)(=O)Nc1nccc(CN2C(=O)Oc3cc(Oc4ncco4)ccc3C23COC3)c1F]

E-101 was synthesized by following similar synthesis sequence and experimental procedures as described for the synthesis of E-97, using key intermediates I-26 and I-35. LCMS (ES) m/z: 492.1 [M+H_{1+.} ¹H NMR (400 MHz, DMSO-d₆): δ 10.41 (s, 1H); 8.02-7.96 (m, 2H); 7.86 (d, J=1.2 Hz, 1H); 7.38 (dd, J₁=2.4 Hz, J₂=8.4 Hz; 1H); 7.23 (d, J=2.4, Hz, 1H); 7.07 (d, J=0.8 Hz, 1H); 7.02-6.98 (m, 1H); 5.10 (s, 2H); 5.08 (d, J=8.0 Hz, 2H); 4.80 (d, J=7.6 Hz, 2H); (d, J=5.2 Hz, 3H).

### Example 102: Preparation of 6-fluoro-3-({2-fluoro-3-(methylsulfamoyl)amino]phenyl}methyl)-7-(1,3-oxazol-2-yloxy)-2,3-dihydrospiro[1,3-benzoxazine-4,3′-oxetan]-2-one (E-102; Compound 73)

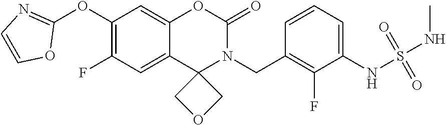 [SMILES: CNS(=O)(=O)Nc1cccc(CN2C(=O)Oc3cc(Oc4ncco4)c(F)cc3C23COC3)c1F]

E-102 was synthesized by following similar synthesis sequence and experimental procedures as described for the synthesis of E-97, using key intermediates I-28 and I-32. LCMS (ES) m/z: 509.3 [M+H]⁺. ¹H NMR (400 MHz, DMSO-d₆): b 9.45 (s, 1H); 8.00 (d, J=10.8 Hz, 1H); 7.87 (s, 1H); 7.51 (d, J=6.8 Hz, 1H); 7.32 (t, J=7.2 Hz, 1H); 7.22 (br s, 1H); 7.10-6.90 (m, 3H); 5.08 (s, 2H); 5.02 (d, J=8.0 Hz, 2H); 4.82 (d, J=8.0 Hz, 2H); 2.52 (d, J=4.8 Hz, 3H).

### Example 103: Preparation of 8-fluoro-3-({2-fluoro-3-(methylsulfamoyl)amino]phenyl}methyl)-7-(1,3-oxazol-2-yloxy)-2,3-dihydrospiro[1,3-benzoxazine-4,3′-oxetan]-2-one (E-103; Compound 82)

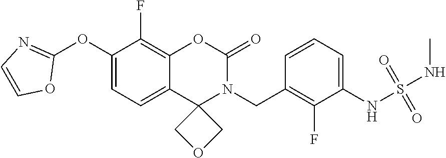 [SMILES: CNS(=O)(=O)Nc1cccc(CN2C(=O)Oc3c(ccc(Oc4ncco4)c3F)C23COC3)c1F]

E-103 was synthesized by following similar synthesis sequence and experimental procedures as described for the synthesis of E-97, using key intermediates I-27 and I-32. LCMS (ES) m/z: 509.3 [M+H]⁺. ¹H NMR (400 MHz, DMSO-d₆): δ 9.45 (s, 1H); 7.88 (d, J=0.8 Hz, 1H); 7.82 (dd, J₁=2.0 Hz, J₂=8.8 Hz, 1H); 7.58-7.54 (m, 1H); 7.37-7.32 (m, 1H); 7.27-7.22 (m, 1H); 7.10-7.02 (m, 3H); 5.10 (s, 2H); 5.07 (d, J=8.0 Hz, 2H); 4.78 (d, J=8.0 Hz, 2H); 2.55 (d, J=4.8 Hz, 3H).

### Example 104: Preparation of 8-fluoro-3-({2-fluoro-3-(methylsulfamoyl)amino]phenyl}methyl)-7-(pyrimidin-2-yloxy)-2,3-dihydrospiro[1,3-benzoxazine-4,3′-oxetan]-2-one (E-104; Compound 88)

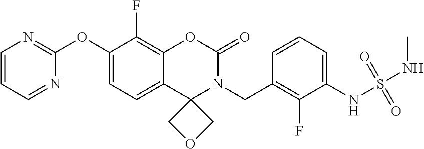 [SMILES: CNS(=O)(=O)Nc1cccc(CN2C(=O)Oc3c(ccc(Oc4ncccn4)c3F)C23COC3)c1F]

E-104 was synthesized by following similar synthesis sequence and experimental procedures as described for the synthesis of E-97, using key intermediates I-27 and I-32. LCMS (ES) m/z: 520.3 [M+H]⁺. ¹H NMR (400 MHz, DMSO-d₆): δ 9.28 (s, 1H); 8.66 (d, J=4.8 Hz, 2H); 7.32 (t, J=4.8 Hz, 1H); 7.30-7.26 (m, 1H); 7.20-7.12 (m, 2H); 7.02-6.90 (m, 2H); 6.82-6.78 (m, 1H); 4.83 (d, J=11.2 Hz, 1H); 4.71 (d, J=11.2 Hz, 1H); 4.67 (s, 2H); 4.27 (d, J=4.0 Hz, 2H); 2.46 (d, J=5.2 Hz, 3H).

### Example 105: Preparation of 6-fluoro-3-({2-fluoro-3-(methylsulfamoyl)amino]phenyl}methyl)-7-(pyrimidin-2-yloxy)-2,3-dihydrospiro[1,3-benzoxazine-4,3′-oxetan]-2-one (E-105; Compound 75)

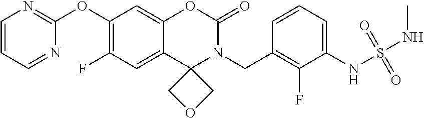 [SMILES: CNS(=O)(=O)Nc1cccc(CN2C(=O)Oc3cc(Oc4ncccn4)c(F)cc3C23COC3)c1F]

E-105 was synthesized by following similar synthesis sequence and experimental procedures as described for the synthesis of E-97, using key intermediates I-28 and I-32. LCMS (ES) m/z: 520.18 [M+H]⁺. ¹H NMR (400 MHz, DMSO-d₆): δ 9.41 (s, 1H); 8.71 (d, J=4.8 Hz, 2H); 7.94 (d, J=10.8 Hz, 1H); 7.39-7.30 (m, 3H); 7.26-7.22 (m, 1H); 7.08 (t, J=8.0 Hz, 1H); 7.00-6.95 (m, 1H); 5.10 (s, 2H); 5.04 (d, J=8.0 Hz, 2H); 5.85 (d, J=8.0 Hz, 2H); 2.55 (d, J=4.8 Hz, 3H).

### Example 106: Preparation of 3-({2-fluoro-3-[(methylsulfamoyl)amino]phenyl}methyl)-7-(pyrazin-2-yloxy)-2,3-dihydrospiro[1,3-benzoxazine-4,3′-oxetan]-2-one (E-106; Compound 70)

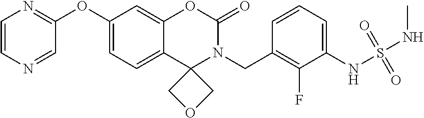 [SMILES: CNS(=O)(=O)Nc1cccc(CN2C(=O)Oc3cc(Oc4cnccn4)ccc3C23COC3)c1F]

E-106 was synthesized by following similar synthesis sequence and experimental procedures as described for the synthesis of E-97, using key intermediates E-97c and chloropyrazine as the starting materials. LCMS (ES) m/z: 502.3 [M+H]⁺. ¹H NMR (400 MHz, DMSO-d₆): δ 9.45 (s, 1H); 8.61 (d, J=1.2 Hz, 1H); 8.43 (d, J=2.8 Hz, 1H); 8.26-8.24 (m, 1H); 7.98 (d, J=8.4 Hz, 1H); 7.35 (t, J=7.6 Hz, 1H); 7.26-7.20 (m, 2H); 7.11-7.04 (m, 2H); 6.59 (t, J=6.8 Hz, 1H); 5.11 (s, 2H); 5.08 (d, J=8.0 Hz, 2H); 4.82 (d, J=8.0 Hz, 2H); 2.56 (d, J=4.8 Hz, 3H).

### Example 107: Preparation of N-(2-fluoro-3-{[2-oxo-7-(pyrazin-2-yloxy)-2,3-dihydrospiro[1,3-benzoxazine-4,3′-oxetan]-3-yl]methyl}phenyl)cyclopropanesulfonamide (E-107; Compound 69)

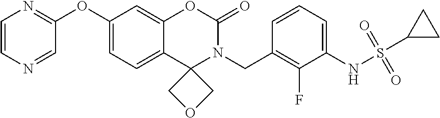 [SMILES: O=C1Oc2cc(Oc3cnccn3)ccc2C2(COC2)N1Cc1cccc(NS(=O)(=O)C2CC2)c1F]

E-107 was synthesized by following similar synthesis sequence and experimental procedures as described for the synthesis of E-97, using key intermediates E-97c and chloropyrazine as the starting materials. (note: cyclopropylsulfonyl chloride was used in the final step). LCMS (ES) m/z: 513.3 [M+H]⁺. ¹H NMR (400 MHz, DMSO-d₆): δ 9.64 (s, 1H); 8.61 (d, J=1.2 Hz, 1H); 8.43 (d, J=2.4 Hz, 1H); 8.24-8.22 (m, 1H); 7.98 (d, J=8.4 Hz, 1H); 7.33 (t, J=7.2 Hz, 1H); 7.22 (dd, J₁=2.4 Hz, J₂=8.8 Hz, 1H); 7.12-7.00 (m, 3H); 5.11 (s, 2H); 5.08 (d, J=8.0 Hz, 2H); 4.81 (d, J=8.0 Hz, 2H); 2.70-2.62 (m, 1H); 0.97-0.87 (m, 4H).

### Example 108: Preparation of 3-({2-fluoro-3-[(methylsulfamoyl)amino]phenyl}methyl)-7-(pyridin-2-yloxy)-2,3-dihydrospiro[1,3-benzoxazine-4,3′-oxetan]-2-one (E-108; Compound 50)

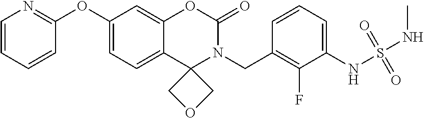 [SMILES: CNS(=O)(=O)Nc1cccc(CN2C(=O)Oc3cc(Oc4ccccn4)ccc3C23COC3)c1F]

E-108 was synthesized by following similar synthesis sequence and experimental procedures as described for the synthesis of E-97, using key intermediates E-97c and 2-bromopyridine as the starting materials. LCMS (ES) m/z: 501.3 [M+H]⁺. ¹H NMR (400 MHz, DMSO-d₆): δ 9.43 (s, 1H); 8.20-8.16 (m, 1H); 7.95-7.88 (m, 2H); 7.35 (t, J=7.2, 1H); 7.27-7.17 (m, 2H); 7.15-7.05 (m, 3H); 6.98-6.92 (m, 2H); 5.10 (s, 2H); 5.07 (d, J=7.6 Hz, 2H); 4.81 (d, J=7.6, 2H); 2.55 (d, J=4.8 Hz, 3H).

### Example 109: Preparation of 3-{[3-fluoro-2-(methylaminosulfonylamino)-4-pyridyl]methyl}-7-(1,2,4-triazin-3-yloxy)-2H,3H-spiro[1,3-benzoxazine-4,3′-oxetan]-2-one (E-109; Compound 10)

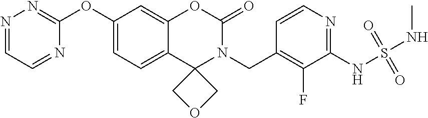 [SMILES: CNS(=O)(=O)Nc1nccc(CN2C(=O)Oc3cc(Oc4nccnn4)ccc3C23COC3)c1F]

E-109 was synthesized by following similar synthesis sequence and experimental procedures as described for the synthesis of E-97, using key intermediates I-26 and I-35. (note: 3-(methylsulfonyl)-1,2,4-triazine was used while O-arylation). LCMS (ES) m/z: 504.3 [M+H]⁺. ¹H NMR (400 MHz, DMSO-d₆): δ 10.41 (s, 1H); 9.31 (d, J=2.4 Hz, 1H); 8.77 (d, J=2.4 Hz, 1H); 8.03 (d, J=8.4 Hz, 1H); 7.99 (d, J=5.2 Hz, 1H); 7.32 (dd, J₁=2.4 Hz, J₂=8.8 Hz, 1H); 7.17 (d, J=2.4 Hz, 1H); 7.02-6.96 (m, 2H); 5.12 (s, 2H); 5.09 (d, J=8.0 Hz, 2H); 4.84 (d, J=8.0 Hz, 2H); 2.53 (d, J=4.8 Hz, 3H).

### Example 110: Preparation of 6-fluoro-3-({2-fluoro-3-[(methylsulfamoyl)amino]phenyl}methyl)-7-(pyridazin-3-yloxy)-2,3-dihydrospiro[1,3-benzoxazine-4,3′-oxetan]-2-one (E-110; Compound 58)

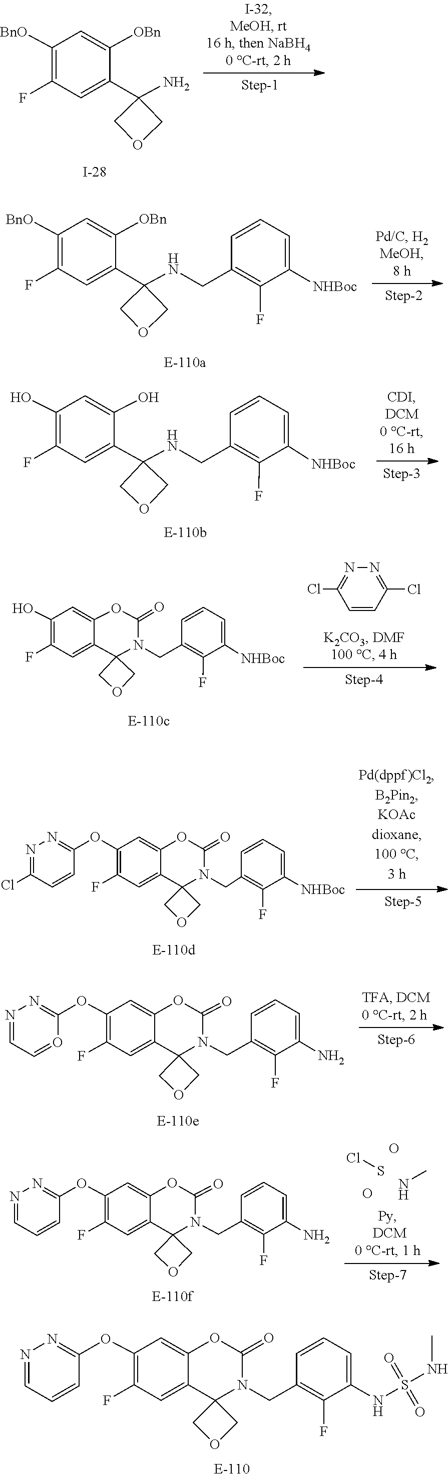

Step-1: Synthesis of tert-butyl N-{3-[({3-[2,4-bis(benzyloxy)-5-fluorophenyl]oxetan-3-yl}amino)methyl]-2-fluorophenyl}carbamate (E-110a): Compound E-110a (3.2 g) was synthesized by following procedure as described for the synthesis of E-01 (step-1) using 3-[2,4-bis(benzyloxy)-5-fluorophenyl]oxetan-3-amine (I-28) (2.45 g, 5.68 mmol) and tert-butyl N-(2-fluoro-3-formylphenyl)carbamate (I-32) (1.36 g, 5.68 mmol) as the starting materials. LCMS (ES) m/z: 601 [M−H]⁺.

Step-2: Synthesis of tert-butyl N-[2-fluoro-3-({[3-(5-fluoro-2,4-dihydroxyphenyl)oxetan-3-yl]amino}methyl)phenyl]carbamate (E-110b): Compound E-110b (1.45 g) was synthesized by following procedure as described for the synthesis of E-60 (step-2) using tert-butyl N-{3-[({3-[2,4-bis(benzyloxy)-5-fluorophenyl]oxetan-3-yl}amino)methyl]-2-fluorophenyl}carbamate (E-110a) (3.2 g, 5.31 mmol) as the starting material. LCMS (ES) m/z: 423.15 [M+H]⁺.

Step-3: Synthesis of tert-butyl N-[2-fluoro-3-({6-fluoro-7-hydroxy-2-oxo-2,3-dihydrospiro[1,3-benzoxazine-4,3′-oxetan]-3-yl}methyl)phenyl]carbamate (E-110c): Compound E-110c (0.81 g) was synthesized by following procedure as described for the synthesis of E-01 (step-2) using tert-butyl N-[2-fluoro-3-({[3-(5-fluoro-2,4-dihydroxyphenyl)oxetan-3-yl]amino}methyl)phenyl]carbamate (E-110b) (1.45 g, 3.43 mmol) as the starting material. LCMS (ES) m/z: 447.1 [M−H]⁺.

Step-4: Synthesis of tert-butyl N-[3-({7-[(6-chloropyridazin-3-yl)oxy]-6-fluoro-2-oxo-2,3-dihydrospiro[1,3-benzoxazine-4,3′-oxetan]-3-yl}methyl)-2-fluorophenyl]carbamate (E-110d): Compound E-110d (0.68 g) was synthesized by following procedure as described for the synthesis of E-32 (step-1) using tert-butyl N-[2-fluoro-3-({6-fluoro-7-hydroxy-2-oxo-2,3-dihydrospiro[1,3-benzoxazine-4,3′-oxetan]-3-yl}methyl)phenyl]carbamate (E-110c) (0.7 g, 1.56 mmol) and 3,6-dichloropyridazine (465 mg, 3.12 mmol) as the starting materials. LCMS (ES) m/z: 559.2 [M−H]⁺.

Step-5: Synthesis of tert-butyl N-(2-fluoro-3-{[6-fluoro-2-oxo-7-(pyridazin-3-yloxy)-2,3-dihydrospiro[1,3-benzoxazine-4,3′-oxetan]-3-yl]methyl}phenyl)carbamate (E-110e): Compound E-110e (0.5 g) was synthesized by following procedure as described for the synthesis of E-32 (step-2) using tert-butyl N-[3-({7-[(6-chloropyridazin-3-yl)oxy]-6-fluoro-2-oxo-2,3-dihydrospiro[1,3-benzoxazine-4,3′-oxetan]-3-yl}methyl)-2-fluorophenyl]carbamate (E-110d) (680 mg, 1.21 mmol) as the starting material. LCMS (ES) m/z: 471.1 [(M-^{t}Bu)+H]⁺.

Step-6: Synthesis of 3-[(3-amino-2-fluorophenyl)methyl]-6-fluoro-7-(pyridazin-3-yloxy)-2,3-dihydrospiro[1,3-benzoxazine-4,3′-oxetan]-2-one (E-110f): Compound E-110f (0.38 g) was synthesized by following procedure as described for the synthesis of E-43 (step-4) using tert-butyl N-(2-fluoro-3-{[6-fluoro-2-oxo-7-(pyridazin-3-yloxy)-2,3-dihydrospiro[1,3-benzoxazine-4,3′-oxetan]-3-yl]methyl}phenyl)carbamate (E-110e) (0.5 g, 950 μmol) as the starting material. LCMS (ES) m/z: 427.1 [M+H]⁺.

Step-7: Synthesis of 6-fluoro-3-({2-fluoro-3-(methylsulfamoyl)amino]phenyl}methyl)-7-(pyridazin-3-yloxy)-2,3-dihydrospiro[1,3-benzoxazine-4,3′-oxetan]-2-one (E-110): Example E-110 (0.063 g) was synthesized by following procedure as described for the synthesis of E-01 (step-6) using 3-[(3-amino-2-fluorophenyl)methyl]-6-fluoro-7-(pyridazin-3-yloxy) 2,3-dihydrospiro[1,3-benzoxazine-4,3′-oxetan]-2-one (E-110f) (180 mg, 422 μmol) as the starting material. The final compound was purified by reverse phase preparative HPLC purification [X-Bridge C-18 (250 mm×4.6 mm, 5 μm particle size) column, water and acetonitrile as mobile phase with 19.0 mL/min flow rate]. LCMS (ES) m/z: 520.3 [M+H]⁺. ¹H NMR (400 MHz, DMSO-d₆): δ 9.45 (s, 1H); 9.07 (d, J=4.4 Hz, 1H); 7.97 (d, J=11.2 Hz, 1H); 7.87-7.83 (m, 1H); 7.66 (d, J=8.8 Hz, 1H); 7.38-7.30 (m, 2H); 7.23 (br s, 1H); 7.08 (t, J=8.0 Hz, 1H); 6.98 (t, J=6.8 Hz, 1H); 5.11 (s, 2H); 5.05 (d, J=8.0 Hz, 2H); 4.86 (d, J=8 Hz, 2H); 2.55 (d, J=4.8 Hz, 3H).

### Example 111: Preparation of 3-({2-fluoro-3-[(methylsulfamoyl)amino]phenyl}methyl)-7-(pyridazin-3-yloxy)-2,3-dihydrospiro[1,3-benzoxazine-4,3′-oxetan]-2-one (E-111; Compound 81)

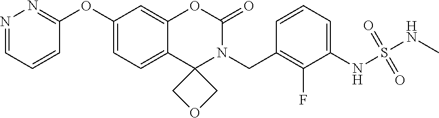 [SMILES: CNS(=O)(=O)Nc1cccc(CN2C(=O)Oc3cc(Oc4cccnn4)ccc3C23COC3)c1F]

E-111 was synthesized by following similar synthesis sequence and experimental procedures as described for the synthesis of E-110, using key intermediates I-26 and I-32. LCMS (ES) m/z: 500.3 [M−H]⁺. ¹H NMR (400 MHz, DMSO-d₆): δ 9.45 (s, 1H); 9.06-9.04 (m, 1H); 7.99 (d, J=8.8 Hz, 1H); 7.83-7.80 (m, 1H); 7.54 (dd, J₁=1.2 Hz, J₂=9.2 Hz, 1H); 7.35 (t, J=6.8 Hz, 1H); 7.26-7.20 (m, 2H); 7.12-7.06 (m, 2H); 6.96 (t, J=6.8 Hz, 1H); 5.17 (s, 2H); 5.09 (d, J=8.0 Hz, 2H); 4.82 (d, J=8.0 Hz, 2H); 2.55 (d, J=4.8 Hz, 3H).

### Example 112: Preparation of 3-({3-[(dimethylsulfamoyl)amino]-2-fluorophenyl}methyl)-7-(pyridazin-3-yloxy)-2,3-dihydrospiro[1,3-benzoxazine-4,3′-oxetan]-2-one (E-112; Compound 64)

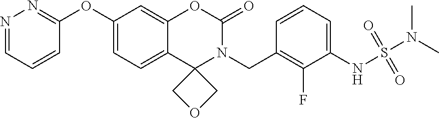 [SMILES: CN(C)S(=O)(=O)Nc1cccc(CN2C(=O)Oc3cc(Oc4cccnn4)ccc3C23COC3)c1F]

E-112 was synthesized by following similar synthesis sequence and experimental procedures as described for the synthesis of E-110, using key intermediates I-26 and I-32. (note: dimethylsulfamoyl chloride was used in the final step). LCMS (ES) m/z: 516.3 [M+H]⁺. ¹H NMR (400 MHz, DMSO-d₆): δ 9.68 (s, 1H); 9.06-9.04 (m, 1H); 7.99 (d, J=8.4 Hz, 1H); 7.83-7.79 (m, 1H); 7.55 (dd, J₁=1.2 Hz, J₂=8.8 Hz, 1H); 7.35 (t, J=6.8 Hz, 1H); 7.23 (dd, J₁=2.4 Hz, J₂=8.4 Hz, 1H); 7.12-6.98 (m, 3H); 5.11 (s, 2H); 5.09 (d, J=8.0 Hz, 2H); 4.82 (d, J=8.0 Hz, 2H); 2.72 (s, 6H).

### Example 113: Preparation of N-(2-fluoro-3-{[2-oxo-7-(pyridazin-3-yloxy)-2,3-dihydrospiro[1,3-benzoxazine-4,3′-oxetan]-3-yl]methyl}phenyl)cyclopropanesulfonamide (E-113; Compound 59)

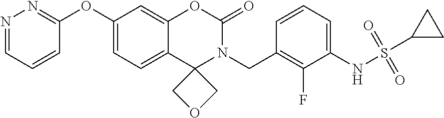 [SMILES: O=C1Oc2cc(Oc3cccnn3)ccc2C2(COC2)N1Cc1cccc(NS(=O)(=O)C2CC2)c1F]

E-113 was synthesized by following similar synthesis sequence and experimental procedures as described for the synthesis of E-110, using key intermediates I-26 and I-32. (note: cyclopropylsulfonyl chloride was used in the final step). LCMS (ES) m/z: 513.0 [M+H]⁺. ¹H NMR (400 MHz, DMSO-d₆): δ 9.66 (s, 1H); 9.06-9.04 (m, 1H); 7.99 (d, J=8.4 Hz, 1H); 7.84-7.80 (m, 1H); 7.55 (dd, J₁=1.2 Hz, J₂=8.8 Hz, 1H); 7.33 (t, J=7.2 Hz, 1H); 7.24 (dd, J=2.4 Hz, J₂=8.8 Hz, 1H); 7.12-7.00 (m, 3H); 5.12 (s, 2H); 5.09 (d, J=8.0 Hz, 2H); 4.82 (d, J=7.6 Hz, 2H); 2.70-2.63 (m, 1H); 1.00-0.88 (m, 4H).

### Example 114: Preparation of 5-fluoro-3-({2-fluoro-3-(methylsulfamoyl)amino]phenyl}methyl)-7-(pyridazin-3-yloxy)-2,3-dihydrospiro[1,3-benzoxazine-4,3′-oxetan]-2-one (E-114; Compound 63)

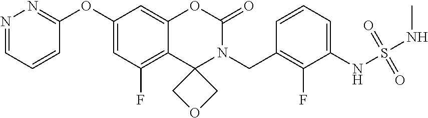 [SMILES: CNS(=O)(=O)Nc1cccc(CN2C(=O)Oc3cc(Oc4cccnn4)cc(F)c3C23COC3)c1F]

E-114 was synthesized by following similar synthesis sequence and experimental procedures as described for the synthesis of E-110, using key intermediates I-29 and I-32. LCMS (ES) m/z: 520.3 [M+H]⁺. ¹H NMR (400 MHz, DMSO-d₆): δ 9.46 (s, 1H); 9.08-9.07 (m, 1H); 7.86-7.82 (m, 1H); 7.57 (dd, J₁=1.2 Hz, J₂=8.8 Hz, 1.2 Hz, 1H); 7.35 (t, J=6.8 Hz, 1H); 7.27-7.23 (m, 2H); 7.14-7.06 (m, 2H); 6.96 (s, 1H); 5.13-5.08 (m, 4H); 4.89 (d, J=8.0 Hz, 2H); 2.55 (d, J=5.2 Hz, 3H).

### Example 115: Preparation of N-(2-fluoro-3-{[6-fluoro-2-oxo-7-(pyridazin-3-yloxy)-2,3-dihydro spiro[1,3-benzoxazine-4,3′-oxetan]-3-yl]methyl}phenyl)cyclopropanesulfonamide (E-115; Compound 53)

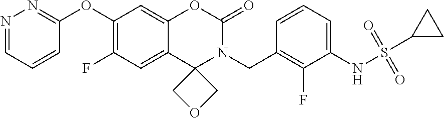 [SMILES: O=C1Oc2cc(Oc3cccnn3)c(F)cc2C2(COC2)N1Cc1cccc(NS(=O)(=O)C2CC2)c1F]

E-115 was synthesized by following similar synthesis sequence and experimental procedures as described for the synthesis of E-110, using key intermediates I-28 and I-32. (note: cyclopropylsulfonyl chloride was used in the final step). LCMS (ES) m/z: 531.3 [M+H]⁺. ¹H NMR (400 MHz, DMSO-d₆): δ 9.65 (s, 1H); 9.07-9.02 (m, 1H); 7.99 (d, J=10.8 Hz, 1H); 7.87-7.54 (m, 2H); 7.35-7.28 (m, 2H); 7.14-7.09 (m, 2H); 5.10 (s, 2H); 5.10 (d, J=8 Hz, 2H); 4.86 (d, J=7.6 Hz, 2H); 2.69 (m, 1H); 0.96-0.85 (m, 4H).

### Example 116: Preparation of 8-fluoro-3-({2-fluoro-3-(methylsulfamoyl)amino]phenyl}methyl)-7-(pyridazin-3-yloxy)-2,3-dihydrospiro[1,3-benzoxazine-4,3′-oxetan]-2-one (E-116; Compound 42)

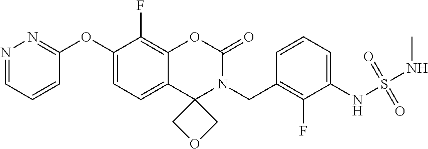 [SMILES: CNS(=O)(=O)Nc1cccc(CN2C(=O)Oc3c(ccc(Oc4cccnn4)c3F)C23COC3)c1F]

E-116 was synthesized by following similar synthesis sequence and experimental procedures as described for the synthesis of E-110, using key intermediates I-27 and I-32. LCMS (ES) m/z: 518.3 [M−H]⁺. ¹H NMR (400 MHz, DMSO-d₆): δ 9.47 (s, 1H); 9.08-9.06 (m, 1H); 7.89-7.80 (m, 2H); 7.68 (dd, J₁=0.8 Hz, J₂=8.8 Hz, 1H); 7.42-7.32 (m, 2H); 7.18 (br s, 1H); 7.10-7.00 (m, 2H); 5.11 (s, 2H); 5.09 (d, J=8.0 Hz, 2H); 4.82 (d, J=7.6 Hz, 2H); 2.55 (d, J=4.4 Hz, 3H).

### Example 117: Preparation of 3-({3-[(dimethylsulfamoyl)amino]-2-fluorophenyl}methyl)-8-fluoro-7-(pyridazin-3-yloxy)-2,3-dihydrospiro[1,3-benzoxazine-4,3′-oxetan]-2-one (E-117; Compound 40)

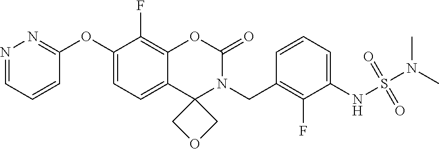 [SMILES: CN(C)S(=O)(=O)Nc1cccc(CN2C(=O)Oc3c(ccc(Oc4cccnn4)c3F)C23COC3)c1F]

E-117 was synthesized by following similar synthesis sequence and experimental procedures as described for the synthesis of E-110, using key intermediates I-27 and I-32. (note: dimethylsulfamoyl chloride was used in the final step). LCMS (ES) m/z: 534.3 [M+H]⁺. ¹H NMR (400 MHz, DMSO-d₆): (9.68 (s, 1H); 9.08-9.06 (m, 1H); 7.88-7.80 (m, 2H); 7.68 (dd, J₁=1.2 Hz, J₂=8.8 Hz, 1.2 Hz, 1H); 7.42-7.32 (m, 2H); 7.12-7.04 (m, 2H); 5.12 (s, 2H); 5.10 (d, J=7.6 Hz, 2H); 4.82 (d, J=8.0 Hz, 2H); 2.71 (s, 6H).

### Example 118: E-118: Preparation of 3-({3-fluoro-2-[(methylsulfamoyl)amino]pyridin-4-yl}methyl)-7-(pyridazin-3-yloxy)-2,3-dihydrospiro[1,3-benzoxazine-4,3′-oxetan]-2-one (E-118; Compound 24)

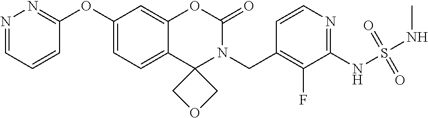 [SMILES: CNS(=O)(=O)Nc1nccc(CN2C(=O)Oc3cc(Oc4cccnn4)ccc3C23COC3)c1F]

E-118 was synthesized by following similar synthesis sequence and experimental procedures as described for the synthesis of E-110, using key intermediates I-26 and I-35. LCMS (ES) m/z: 503.1 [M+H]⁺. ¹H NMR (400 MHz, DMSO-d₆): δ 10.39 (s, 1H); 9.05 (d, J=3.6 Hz, 1H); 8.04-7.96 (m, 2H); 7.86-7.78 (m, 1H); 7.54 (d, J=8.8 Hz, 1H); 7.23 (d, J=7.6 Hz, 1H); 7.06 (s, 1H); 7.00-6.90 (m, 2H); 5.10 (s, 2H); 5.09 (d, J=8.0 Hz, 2H); 4.83 (d, J=7.6 Hz, 2H); 2.34 (d, J=5.2 Hz, 3H).

### Example 119: Preparation of 6-fluoro-3-{[3-fluoro-2-(methylaminosulfonylamino)-4-pyridyl]methyl}-7-(3-pyridazinyloxy)-2H,3H-spiro[1,3-benzoxazine-4,3′-oxetan]-2-one (E-119; Compound 18)

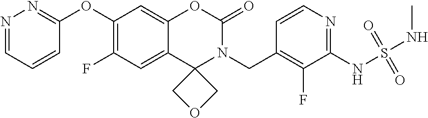 [SMILES: CNS(=O)(=O)Nc1nccc(CN2C(=O)Oc3cc(Oc4cccnn4)c(F)cc3C23COC3)c1F]

E-119 was synthesized by following similar synthesis sequence and experimental procedures as described for the synthesis of E-110, using key intermediates I-28 and I-35. LCMS (ES) m/z: 521.3 [M+H]⁺. ¹H NMR (400 MHz, DMSO-d₆): δ 10.39 (s, 1H); 9.07 (d, J=3.6 Hz, 1H); 8.02-7.98 (m, 2H); 7.87-7.83 (m, 1H); 7.65 (d, J=8.0 Hz, 1H); 7.32 (d, J=6.8 Hz, 1H); 7.04-6.96 (m, 2H); 5.12 (s, 2H); 5.07 (d, J=8.0 Hz, 2H); 4.88 (d, J=8.0 Hz, 2H); 2.53 (d, J=4.4 Hz, 3H).

### Examples 120 and 121: Preparations of 3′-({2-fluoro-3-(methylsulfamoyl)amino]phenyl}methyl)-2′-oxo-2′,3′-dihydrospiro[azetidine-3,4′-[1,3]benzoxazin]-7′-yl N,N-dimethylcarbamate and 3′-(2-fluoro-3-((N-methylsulfamoyl)amino)benzyl)-1-methyl-2′-oxo-2′,3′-dihydrospiro[azetidine-3,4′-benzo[e][1,3]oxazin]-7′-yl dimethylcarbamate (E-120 and E-121; Compounds 49 and 48, respectively)

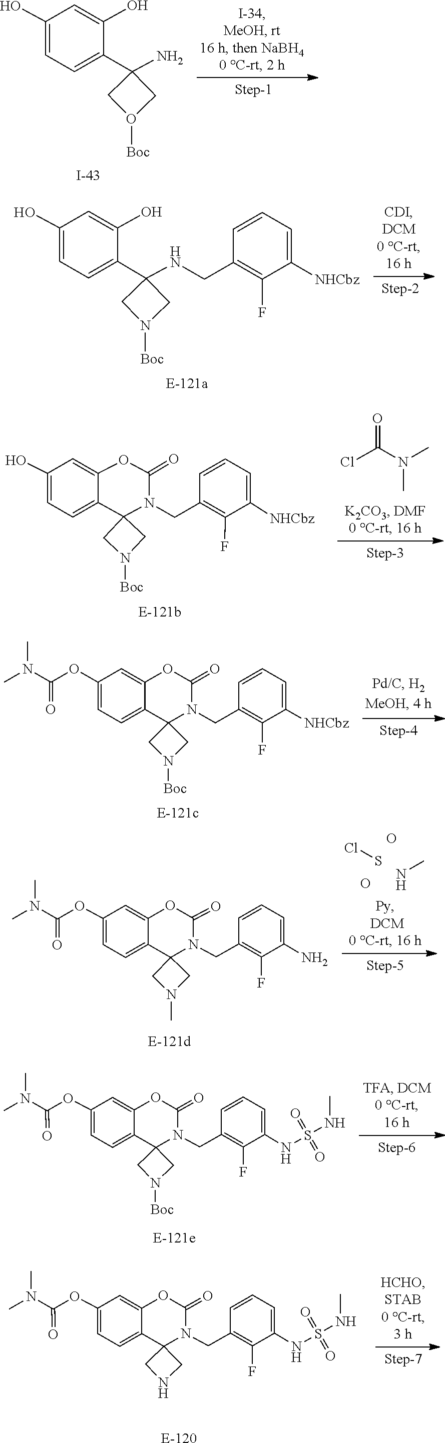

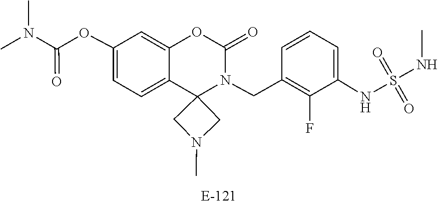

Step-1: Synthesis of tert-butyl 3-{[(3-{[(benzyloxy)carbonyl]amino}-2-fluorophenyl)methyl]amino}-3-(2,4-dihydroxyphenyl)azetidine-1-carboxylate (E112a): Compound E-121a (1.2 g) was synthesized by following procedure as described for the synthesis of E-01 (step-1) using tert-butyl 3-amino-3-(2,4-dihydroxyphenyl)azetidine-1-carboxylate (I-43) (1.8 g, 6.42 mmol) and benzyl N-(2-fluoro-3-formylphenyl)carbamate (I-34) (1.75 g, 6.42 mmol) as the starting materials. LCMS (ES) m/z: 538.6 [M+H]⁺.

Step-2: Synthesis of tert-butyl 3′-[(3-{[(benzyloxy)carbonyl]amino}-2-fluorophenyl)methyl]-7′-hydroxy-2′-oxo-2′,3′-dihydrospiro[azetidine-3,4′-[1,3]benzoxazine]-1-carboxylate (E-121b): Compound E-121b (1.05 g) was synthesized by following procedure as described for the synthesis of E-01 (step-2) using tert-butyl 3-{[(3-{[(benzyloxy)carbonyl]amino}-2-fluorophenyl)methyl]amino}-3-(2,4-dihydroxyphenyl)azetidine-1-carboxylate (E-121a) (1.2 g, 2.23 mmol) as the starting material. LCMS (ES) m/z: 564.33 [M+H]⁺.

Step-3: Synthesis of tert-butyl 3′-[(3-{[(benzyloxy)carbonyl]amino}-2-fluorophenyl)methyl]-7′-[(dimethylcarbamoyl)oxy]-2′-oxo-2′,3′-dihydrospiro[azetidine-3,4′-[1,3]benzoxazine]-1-carboxylate (E-121c): Compound E-121c (0.6 g) was synthesized by following procedure as described for the synthesis of E-01 (step-4) using tert-butyl 3′-[(3-{[(benzyloxy)carbonyl]amino}-2-fluorophenyl)methyl]-7′-hydroxy-2′-oxo-2′,3′-dihydrospiro[azetidine-3,4′-[1,3]benzoxazine]-1-carboxylate (E-121b) (1.05 g, 1.86 mmol) as the starting material. LCMS (ES) m/z: 535.28 [(M-Boc)+H]⁺.

Step-4: Synthesis of tert-butyl 3′-[(3-amino-2-fluorophenyl)methyl]-7′-(dimethylcarbamoyl)oxy]-2′-oxo-2′,3′-dihydrospiro[azetidine-3,4′-[1,3]benzoxazine]-1-carboxylate (E-121d): To a stirred solution of tert-butyl 3′-[(3-{[(benzyloxy)carbonyl]amino}-2-fluorophenyl)methyl]-7′-[(dimethylcarbamoyl)oxy]-2′-oxo-2′,3′-dihydrospiro[azetidine-3,4′-[1,3]benzoxazine]-1-carboxylate (E-121c) (0.6 g, 0.945 mmol) in MeOH (20 mL) was added 10% Pd/C (0.565 g, 5.31 mmol) and the reaction mixture was stirred at room temperature under H₂ atmosphere. After complete consumption of starting material (4 h), the catalyst was filtered off through a celite pad and washed with MeOH (5 mL×2). MeOH was then evaporated under reduced pressure and the residue was purified by Combi-Flash (using gradient elution of 0-70% of EtOAc in heptane) to afford the desired compound E-121d (0.5 g) as an off white solid. LCMS (ES) m/z: 501.3 [M+H]⁺.

Step-5: Synthesis of tert-butyl 7′-[(dimethylcarbamoyl)oxy]-3′-({2-fluoro-3-(methylsulfamoyl)amino]phenyl}methyl)-2′-oxo-2′,3′-dihydrospiro[azetidine-3,4′-[1,3]benzoxazine]-1-carboxylate (E-121e): Compound E-121e (0.25 g) was synthesized by following procedure as described for the synthesis of E-01 (step-6) using tert-butyl 3′-[(3-amino-2-fluorophenyl)methyl]-7′-[(dimethylcarbamoyl)oxy]-2′-oxo-2′,3′-dihydrospiro[azetidine-3,4′-[1,3]benzoxazine]-1-carboxylate (E-121d) (0.3 g, 0.59 mmol) as the starting material. LCMS (ES) m/z: 592.37 [M−H]⁺.

Step-6: Synthesis of 3′-(12-fluoro-3-[(methylsulfamoyl)amino]phenyl}methyl)-2′-oxo-2′,3′-dihydrospiro[azetidine-3,4′-[1,3]benzoxazin]-7′-yl N,N-dimethylcarbamate (E-120): To a stirred solution of tert-butyl 7′-[(dimethylcarbamoyl)oxy]-3′-({2-fluoro-3-(methylsulfamoyl)amino]phenyl}methyl)-2′-oxo-2′,3′-dihydrospiro[azetidine-3,4′-[1,3]benzoxazine]-1-carboxylate (E-121e) (0.18 g, 0.303 mmol) in DCM (3 mL) was added trifluoroacetic acid (0.138 g, 1.21 mmol) at 0° C. and the reaction mixture was then allowed to warm to room temperature over 16 h. After completion of the reaction, volatiles were removed under reduced pressure and the residue was washed with diethyl ether (3×5 mL) to afford desired compound E-120 (0.011 g) as a white solid. LCMS (ES) m/z: 494.04 [M+H]⁺.

Step-7: Synthesis of 3′-(12-fluoro-3-[(methylsulfamoyl)amino]phenyl}methyl)-1-methyl-2′-oxo-2′,3′-dihydrospiro[azetidine-3,4′-[1,3]benzoxazin]-7′-yl N,N-dimethylcarbamate (E-121): To a stirred solution of 3′-({2-fluoro-3-(methylsulfamoyl)amino]phenyl}methyl)-2′-oxo-2′,3′-dihydrospiro[azetidine-3,4′-[1,3]benzoxazin]-7′-yl N,N-dimethylcarbamate (E-120) (0.15 g, 0.30 mmol) in DCM (3 mL) was added formaldehyde (0.027 g, 0.91 mmol) and sodium triacetoxyborohydride (0.258 g, 1.22 mmol) at 0° C. The reaction mixture was stirred at room temperature and the reaction progress was monitored by TLC/LCMS analysis. After completion of the reaction (3 h), water (20 mL) was added to it and extraction was carried out using DCM (20 mL×2). The combined organic extracts were dried over anhydrous Na₂SO₄, filtered and concentrated under reduced pressure. The residue was purified by reverse phase preparative HPLC [using Xselect Phenyl Hexyl 19 mm×250 mm column, 5 m particle size) column, ammonium bicarbonate and acetonitrile as mobile phase] to afford desired compound E-121 (0.015 g) as a white solid. LCMS (ES) m/z: 508.14 [M+H]⁺. ¹H NMR (400 MHz, DMSO-d₆): (9.47 (s, 1H); 7.90 (d, J=8.8 Hz, 1H); 7.34-7.30 (m, 1H); 7.27 (q, J=5.2 Hz, 1H); 7.10-7.04 (m, 2H); 6.90 (d, J=2.4 Hz, 1H); 6.85-6.82 (m, 1H); 5.12 (s, 2H); 3.71 (d, J=8.4 Hz, 2H); 3.53 (d, J=8.4 Hz, 2H); 3.05 (s, 3H); 2.92 (s, 3H); 2.53 (d, J=4.4 Hz, 3H); 2.36 (s, 3H).

### Example 122: Preparation of 3-({2-fluoro-3-[(methylsulfamoyl)amino]phenyl}methyl)-2-oxo-2,3-dihydrospiro[1,3-benzoxazine-4,1′-cyclobutan]-7-yl N,N-dimethylcarbamate (E-122; Compound 38)

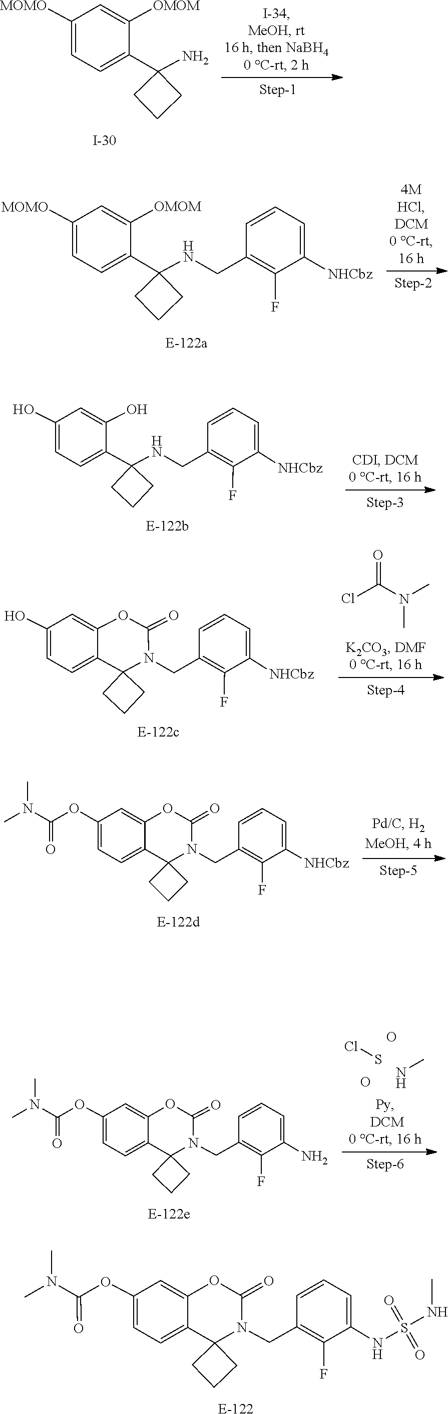

Step-1: Synthesis of benzyl (3-(((1-(2,4-bis(methoxymethoxy)phenyl)cyclobutyl)amino) methyl)-2-fluorophenyl)carbamate (E-122a): Compound E-122a (2 g) was synthesized by following procedure as described for the synthesis of E-01 (step-1) using 1-[2,4-bis(methoxymethoxy)phenyl]cyclobutan-1-amine (I-30) (1.86 g, 6.95 mmol) and benzyl (2-fluoro-3-formylphenyl)carbamate (1-34) (2 g, 3.81 mmol) as the starting materials. ¹H NMR (400 MHz, DMSO-d₆): δ 9.32 (s, 1H); 7.46 (s, 1H); 7.38-7.31 (m, 5H); 7.10-7.00 (m, 3H); 6.67 (s, 1H); 6.61 (d, J=8 Hz, 1H); 5.15-5.11 (m, 6H); 3.36-3.30 (m, 6H); 3.25-3.15 (m, 3H); 2.30-2.27 (m, 2H); 2.20-2.11 (m, 3H); 1.70-1.69 (m, 1H).

Step-2: Synthesis of benzyl N-[3-(1[1-(2,4-dihydroxyphenyl)cyclobutyl]amino}methyl)-2-fluorophenyl]carbamate (E-122b): To a stirred solution of benzyl N-{3-[({1-[2,4-bis(methoxymethoxy)phenyl]cyclobutyl}amino)methyl]-2-fluorophenyl}carbamate (E-122a) (1.9 g, 3.62 mmol) in MeOH (20 mL) was added a solution of 4 M HCl in 1,4-dioxane (18 mL) at 0° C. The reaction mixture was stirred at room temperature and the reaction progress was monitored by TLC/LCMS analysis. After completion (16 h), volatiles were removed under reduced pressure and saturated sodium bicarbonate solution (50 mL) was added to the residue. Extraction was carried out using EtOAc (50 mL×2); the organic extracts were washed with brine (50 mL), dried over anhydrous sodium sulfate, filtered and concentrated. The residue was triturated with diethyl ether (15 mL) to afford desired compound E-122b (1.5 g) as an off-white solid. LCMS (ES) m/z: 437.1 [M+H]⁺.

Step-3: Synthesis of benzyl N-[2-fluoro-3-({7-hydroxy-2-oxo-2,3-dihydrospiro[1,3-benzoxazine-4,1′-cyclobutan]-3-yl}methyl)phenyl]carbamate (E-122c): Compound E-122c (1.4 g) was synthesized by following procedure as described for the synthesis of E-01 (step-2) using benzyl N-[3-({[1-(2,4-dihydroxyphenyl)cyclobutyl]amino}methyl)-2-fluorophenyl]carbamate (E-122b) (1.5 g, 3.44 mmol) as the starting material. LCMS (ES) m/z: 463.2 [M+H]⁺.

Step-4: Synthesis of benzyl N-[3-({7-[(dimethylcarbamoyl)oxy]-2-oxo-2,3-dihydrospiro[1,3-benzoxazine-4,1′-cyclobutan]-3-yl}methyl)-2-fluorophenyl]carbamate (E-122d): Compound E-122d (0.4 g) was synthesized by following procedure as described for the synthesis of E-01 (step-4) using benzyl N-[2-fluoro-3-({7-hydroxy-2-oxo-2,3-dihydrospiro[1,3-benzoxazine-4,1′-cyclobutan]-3-yl}methyl)phenyl]carbamate (E-122c) (0.359 g, 0.865 mmol) as the staring material. LCMS (ES) m/z: 534.2 [M+H]⁺.

Step-5: Synthesis of benzyl N-[2-fluoro-3-({7-hydroxy-2-oxo-2,3-dihydrospiro[1,3-benzoxazine-4,1′-cyclobutan]-3-yl}methyl)phenyl]carbamate (E-122e): To a stirred solution of N-[3-({7-[(dimethylcarbamoyl)oxy]-2-oxo-2,3-dihydrospiro[1,3-benzoxazine-4,1′-cyclobutan]-3-yl}methyl)-2-fluorophenyl]carbamate (E-122d) (0.4 g, 0.750 mmol) in MeOH (10 mL) was added 10% Pd/C (40 mg) and the reaction mixture was allowed to stir at room temperature under hydrogen atmosphere. The reaction progress was monitored by TLC/LCMS analysis. After completion of the reaction (4 h), the catalyst was filtered off through celite and washed with MeOH (5 mL×2). The filtrate was concentrated under reduced pressure and the obtained solid was triturated with diethyl ether (10 mL) to afford desired compound E-122e (0.250 g) as an off-white solid. LCMS (ES) m/z: 400.3 [M+H]⁺.

Step-6: Synthesis of 3-({2-fluoro-3-[(methylsulfamoyl)amino]phenyl}methyl)-2-oxo-2,3-dihydrospiro[1,3-benzoxazine-4,1′-cyclobutan]-7-yl N,N-dimethylcarbamate (E-122): Example E-122 (0.085 g) was synthesized by following procedure as described for the synthesis of E-01 (step-6) using 3-[(3-amino-2-fluorophenyl)methyl]-2-oxo-2,3-dihydrospiro[1,3-benzoxazine-4,1′-cyclobutan]-7-yl N,N-dimethylcarbamate (E-122e) (0.150 g, 0.376 mmol) as the starting material. The final compound was purified by reverse phase preparative HPLC purification [using Xbridge-C18 (19 mm×250 mm, 5 μm particle size) column, water and acetonitrile as mobile phase with 19.0 mL/min flow rate]. LCMS (ES) m/z: 491.3 [M−H]⁺. ¹H NMR, (400 MHz, DMSO-d₆) δ 9.42 (s, 1H); 8.39 (d, J=8.7 Hz, 1H); 7.31 (t, J=7.6 Hz, 1H); 7.22-7.21 (m, 1H); 7.07-7.03 (m, 2H); 6.94 (d, J=2.0 Hz, 1H); 6.75 (t, J=7.6 Hz, 1H); 4.96 (s, 2H); 3.05 (s, 3H); 2.92 (s, 3H); 2.87-2.77 (m, 2H); 2.53 (s, 3H); 2.33-2.30 (m, 2H); 2.02-1.93 (m, 2H).

### Example 123: Preparation of 3-({3-[(dimethylsulfamoyl)amino]-2-fluorophenyl}methyl)-2-oxo-2,3-dihydrospiro[1,3-benzoxazine-4,1′-cyclobutan]-7-yl N,N-dimethyl carbamate (E-123; Compound 33)

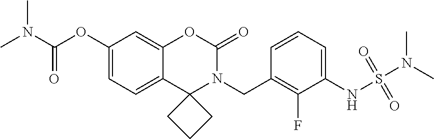

E-123 was synthesized by following synthesis sequence and procedures as described for the synthesis of E-122, using key intermediate E-122e and dimethylsulfamoylchloride as the starting materials. LCMS (ES) m/z: 507.4 [M+H]. ¹H NMR (400 MHz, DMSO-d₆): δ 9.64 (s, 1H); 7.70 (d, J=8.8 Hz, 1H); 7.32 (t, J=7.2 Hz, 1H); 7.10-7.02 (m, 2H); 6.94 (d, J=2.4 Hz, 1H); 6.85 (t, J=6.8 Hz, 1H); 4.97 (s, 2H); 3.09 (s, 3H); 2.92 (s, 3H); 2.82-2.76 (m, 2H); 2.67 (s, 6H); 2.34-2.28 (m, 2H); 1.98-1.92 (m, 2H).

### Example 124: Preparation of 3-({2-fluoro-3-[(methylsulfamoyl)amino]phenyl}methyl)-7-(pyridazin-3-yloxy)-2,3-dihydrospiro[1,3-benzoxazine-4,1′-cyclobutan]-2-one (E-124; Compound 41)

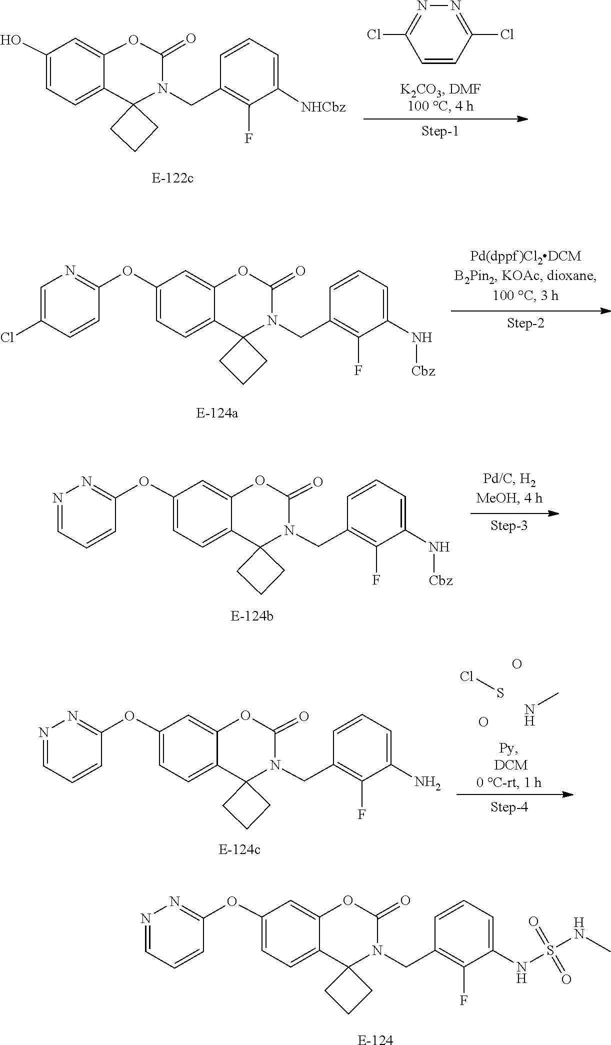

Step-1: Synthesis of benzyl N-[3-({7-[(6-chloropyridazin-3-yl)oxy]-2-oxo-2,3-dihydrospiro [1,3-benzoxazine-4,1′-cyclobutan]-3-yl}methyl)-2-fluorophenyl]carbamate (E-124a): Compound E-124a (1 g) was synthesized by following procedure as described for the synthesis of E-32 (step-1) using benzyl N-[2-fluoro-3-({7-hydroxy-2-oxo-2,3-dihydrospiro[1,3-benzoxazine-4,1′-cyclobutan]-3-yl}methyl)phenyl]carbamate (E-122c) (1 g, 2.16 mmol) and 3,6-dichloropyridazine (387 mg, 2.59 mmol) as the starting materials. LCMS (ES) m/z: 575.1 [M+H]⁺.

Step-2: Synthesis of benzyl N-(2-fluoro-3-{[2-oxo-7-(pyridazin-3-yloxy)-2,3-dihydrospiro[1,3-benzoxazine-4,1′-cyclobutan]-3-yl]methyl}phenyl)carbamate (E-124b): Compound E-124b (0.4 g) was synthesized by following procedure as described for the synthesis of E-32 (step-2) using benzyl N-[3-({7-[(6-chloropyridazin-3-yl)oxy]-2-oxo-2,3-dihydrospiro[1,3-benzoxazine-4,1′-cyclobutan]-3-yl}methyl)-2-fluorophenyl]carbamate (E-124a) (1 g, 1.74 mmol) as the starting material. LCMS (ES) m/z: 541.2 [M+H]⁺.

Step-3: Synthesis of 3-[(3-amino-2-fluorophenyl)methyl]-7-(pyridazin-3-yloxy)-2,3-dihydrospiro[1,3-benzoxazine-4,1′-cyclobutan]-2-one (E-124c): Compound E-124c (0.250 g) was synthesized by following procedure as described for the synthesis of E-122 (step-5) using benzyl N-(2-fluoro-3-{[2-oxo-7-(pyridazin-3-yloxy)-2,3-dihydrospiro[1,3-benzoxazine-4,1′-cyclobutan]-3-yl]methyl}phenyl)carbamate (E-124b) (0.4 g, 0.740 mmol) as the starting material. LCMS (ES) m/z: 407.2 [M+H]⁺.

Step-4: Synthesis of 3-({2-fluoro-3-[(methylsulfamoyl)amino]phenyl}methyl)-7-(pyridazin-3-yloxy)-2,3-dihydrospiro[1,3-benzoxazine-4,1′-cyclobutan]-2-one (E-124): Example E-124 (0.140 g) was synthesized by following procedure as described for the synthesis of E-01 (step-6) using 3-[(3-amino-2-fluorophenyl)methyl]-7-(pyridazin-3-yloxy)-2,3-dihydrospiro[1,3-benzoxazine-4,1′-cyclobutan]-2-one (E-124c) (250 mg, 0.615 mmol) as the starting material. The final compound was purified by reverse phase preparative HPLC purification [using Sunfire C18 (19 mm×250 mm, 5 m particle size) column, water and acetonitrile as mobile phase with 19.0 mL/min flow rate]. LCMS (ES) m/z: 498.3 [M−H]⁺. ¹H NMR (400 MHz, DMSO-d₆): δ 9.44 (s, 1H); 9.05 (d, J=4.4 Hz, 1H); 7.82-7.78 (m, 2H); 7.53 (d, J=8.8 Hz, 1H); 7.33 (t, J=7.6 Hz, 1H); 7.24-7.15 (m, 2H); 7.10-7.04 (m, 2H); 6.79 (t, J=6.8 Hz, 1H); 4.98 (s, 2H); 2.86-2.78 (m, 2H); 2.53 (d, J=4.8 Hz, 3H); 2.40-2.32 (m, 2H); 2.02-1.94 (m, 2H).

### Example 125: Preparation of 3-{[3-(dimethylaminosulfonylamino)-2-fluorophenyl]methyl}-7-(3-pyridazinyloxy)-2H,3H-spiro[1,3-benzoxazine-4,1′-cyclobutan]-2-one (E-125; Compound 25)

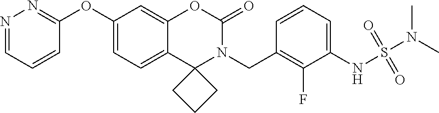 [SMILES: CN(C)S(=O)(=O)Nc1cccc(CN2C(=O)Oc3cc(Oc4cccnn4)ccc3C23CCC3)c1F]

E-125 was synthesized by following similar synthesis sequence and experimental procedures as described for the synthesis of E-124, using key intermediates E-124c and dimethylsulfamoyl chloride as the starting materials. LCMS (ES) m/z: 514.4 [M+H]⁺. 1H NMR (400 MHz, DMSO-d₆): δ 9.67 (s, 1H); 9.05 (dd, J₁=0.8 Hz, J₂=4.4 Hz, 1H); 7.83-7.77 (m, 2H); 7.55 (dd, J₁=0.8 Hz, J₂=8.8 Hz, 1H); 7.33 (t, J=7.6 Hz, 1H); 7.16 (dd, J₁=2.4 Hz, J₂=8.8 Hz, 1H); 7.10-7.04 (m, 2H); 6.90 (t, J=6.8 Hz, 1H); 5.00 (s, 2H); 2.88-2.78 (m, 2H); 2.69 (s, 6H); 2.40-2.30 (m, 2H); 2.02-1.95 (m, 2H).

### Example 126: Preparation of 6-fluoro-3-{1[2-fluoro-3-(methylaminosulfonylamino)phenyl]methyl}-7-(3-pyridazinyloxy)-2H,3H-spiro[1,3-benzoxazine-4,1′-cyclobutan]-2-one (E-126; Compound 19)

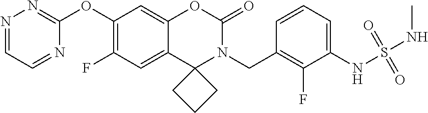 [SMILES: CNS(=O)(=O)Nc1cccc(CN2C(=O)Oc3cc(Oc4nccnn4)c(F)cc3C23CCC3)c1F]

E-126 was synthesized by following similar synthesis sequence and experimental procedures as described for the synthesis of E-124, using key intermediates I-40 and I-34. LCMS (ES) m/z: 518.4 [M+H]⁺. ¹H NMR (400 MHz, DMSO-d₆): δ 9.42 (s, 1H); 9.06 (dd, J₁=1.2 Hz, J₂=4.4 Hz, 1H); 7.86-7.78 (m, 2H); 7.65 (dd, J₁=1.2 Hz, J₂=8.8 Hz, 1H); 7.38-7.30 (m, 2H); 7.24-7.18 (m, 1H); 7.09 (t, J=8.0 Hz, 1H); 6.83 (t, J=6.8 Hz, 1H); 4.98 (s, 2H); 2.85-2.76 (m, 2H); 2.53 (d, J=4.8 Hz, 3H); 2.42-2.34 (m, 2H); 2.02-1.90 (m, 2H).

### Example 127: Preparation of 3-({2-fluoro-3-[(methylsulfamoyl)amino]phenyl}methyl)-7-(pyrimidin-2-yloxy)-3,4-dihydro-2H-1,3-benzoxazin-2-one (E-127; Compound 17)

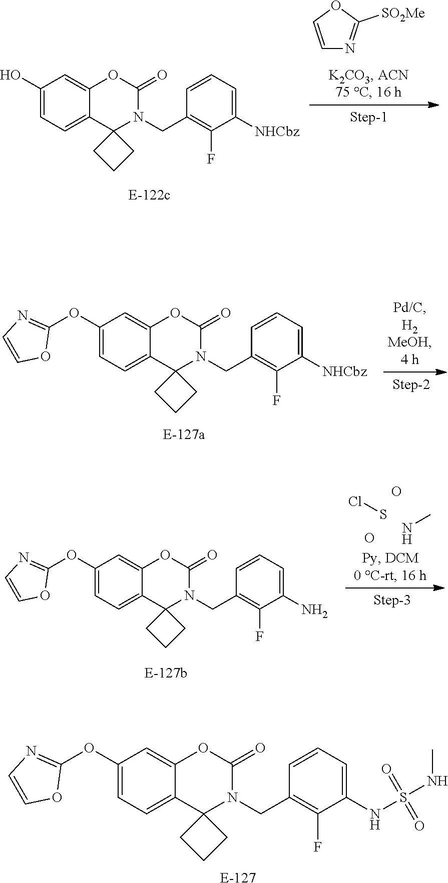

Step-1: Synthesis of tert-butyl (2-fluoro-3-(((2-hydroxy-4-((4-methoxybenzyl)oxy) benzyl)amino)methyl)phenyl)carbamate (E-127a): To a stirred solution of benzyl-2-fluoro-3-{(7-hydroxy-2-oxo-2H,3H-spiro[1,3-benzoxazine-4,1′-cyclobutan]-3-yl)methyl}phenylaminoformylate (122c) (0.5 g, 1.08 mmol) in acetonitrile (4 mL) were added K₂CO₃ (0.29 g, 2.16 mmol), 2-mesyl-1,3-oxazole (0.31 g, 2.16 mmol) and the reaction mixture was allowed to stir at 75° C. The reaction progress was monitored by TLC/LCMS analysis. After completion of the reaction (16 h), the reaction mixture was cooled to room temperature and then quenched with water (30 mL). Extraction was carried out using EtOAc (30 mL×3); the combined organic extracts were washed with water (50 mL), brine (50 mL), dried over anhydrous Na₂SO₄, filtered and concentrated under reduced pressure. The residue was purified by Combi-Flash (using gradient elution of 0-10% of MeOH in DCM) to afford desired compound 127a (0.4 g) as a pale yellow solid. MS (ES) m/z: 530.2 [M+H]⁺.

Step-2: Synthesis of tert-butyl (2-fluoro-3-(((2-hydroxy-4-((4-methoxybenzyl)oxy) benzyl)amino)methyl)phenyl)carbamate (E-127b): Compound E-127b (0.09 g) was synthesized by following procedure as described for the synthesis of E-122 (step-5) using benzyl-2-fluoro-3-({7-(1,3-oxazol-2-yloxy)-2-oxo-2H,3H-spiro[1,3-benzoxazine-4,1′-cyclobutan]-3-yl}methyl)phenylaminoformylate (127a) (0.4 g, 0.75 mmol) as the starting material. LCMS (ES) m/z: 395.9 [M+H]⁺.

Step-3: Synthesis of tert-butyl (2-fluoro-3-(((2-hydroxy-4-((4-methoxybenzyl)oxy) benzyl)amino)methyl)phenyl)carbamate (E-127): Example 127 (0.027 g) was synthesized by following procedure as described for the synthesis of E-01 (step-6) using 2-fluoro-3-({7-(1,3-oxazol-2-yloxy)-2-oxo-2H,3H-spiro[1,3-benzoxazine-4,1′-cyclobutan]-3-yl}methyl)aniline (E-127b) (0.08 g, 0.20 mmol) as the starting material. The final compound was purified by reverse phase preparative HPLC purification [using Sunfire C18 (19 mm×250 mm, 5 m particle size) column, water and acetonitrile as mobile phase with 19.0 mL/min flow rate]. LCMS (ES) m/z: 489.3 [M+H]⁺. ¹H NMR (400 MHz, DMSO d₆): δ 9.41 (s, 1H); 7.85 (s, 1H); 7.80 (d, J=8.8 Hz, 1H); 7.36-7.16 (m, 4H); 7.08-7.02 (m, 2H); 6.77 (d, J=6.8 Hz, 1H); 4.97 (s, 2H); 2.84-2.76 (m, 2H); 2.53 (d, J=4.4 Hz, 3H); 2.38-2.30 (m, 2H); 2.00-1.90 (m, 2H).

### Example 128: Preparation of 3-{[3-fluoro-2-(methylaminosulfonylamino)-4-pyridyl]methyl}-7-(3-pyridazinyloxy)-2H,3H-spiro[1,3-benzoxazine-4,1′-cyclobutan]-2-one (E-128; Compound 20)

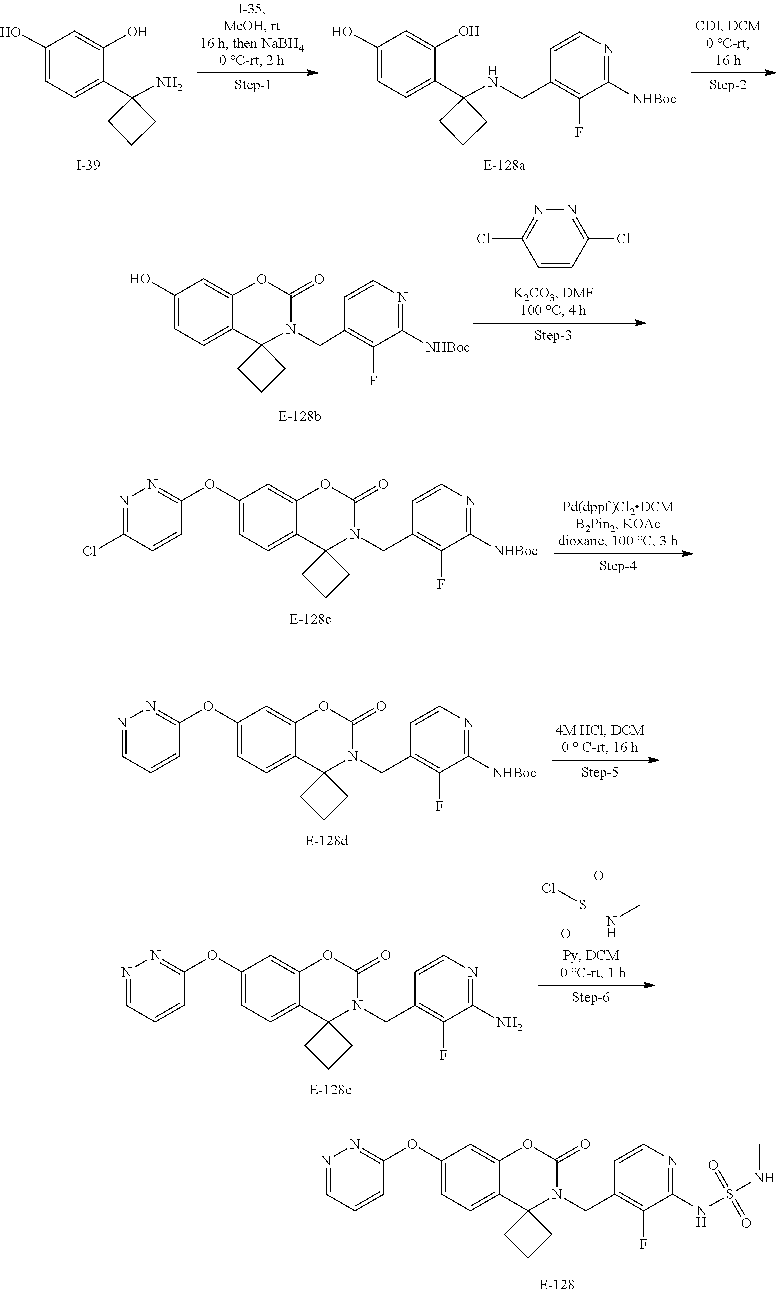

Step-1: Synthesis of tert-butyl (4-(((1-(2,4-dihydroxyphenyl)cyclobutyl)amino)methyl)-3-fluoropyridin-2-yl)carbamate (E-128a): Compound E-128a (1.8 g) was synthesized by following procedure as described for the synthesis of E-01 (step-1) using 4-(1-aminocyclobutyl)resorcinol (I-39) (1.8 g, 10 mmol) and tert-butyl-3-fluoro-4-formyl-2-pyridylaminoformylate (I-35) (2.41 g, 10 mmol) as the starting materials. LCMS (ES) m/z: 404.1 [M+H]⁺.

Step-2: Synthesis of tert-butyl (3-fluoro-4-((7-hydroxy-2-oxospiro[benzo[e][1,3]oxazine-4,1′-cyclobutan]-3(2H)-yl)methyl)pyridin-2-yl)carbamate (E-128b): Compound E-128b (0.9 g) was synthesized by following procedure as described for the synthesis of E-01 (step-2) using tert-butyl-4-{[1-(2,4-dihydroxyphenyl)cyclobutylamino]methyl}-3-fluoro-2-pyridylaminoformylate (E-128a) (1.8 g, 4.46 mmol) as the starting material. LCMS (ES) m/z: 430.1 [M+H]⁺.

Step-3: Synthesis of tert-butyl (4-((7-((6-chloropyridazin-3-yl)oxy)-2-oxospiro[benzo[e][1,3]oxazine-4,1′-cyclobutan]-3(2H)-yl)methyl)-3-fluoropyridin-2-yl)carbamate (E-128c): Compound E-128c (0.8 g) was synthesized by following procedure as described for the synthesis of E-34 (step-1) using tert-butyl-3-fluoro-4-{(7-hydroxy-2-oxo-2H,3H-spiro[1,3-benzoxazine-4,1′-cyclobutan]-3-yl)methyl}-2-pyridylaminoformylate (E-128b) (0.9 g, 2.1 mmol) and 3,6-dichloropyridazine (468 mg, 3.14 mmol) as the starting materials. LCMS (ES) m/z: 442.1 [(M-Boc)+H]⁺.

Step-4: Synthesis of tert-butyl (3-fluoro-4-((2-oxo-7-(pyridazin-3-yloxy)spiro[benzo[e][1,3]oxazine-4,1′-cyclobutan]-3(2H)-yl)methyl)pyridin-2-yl)carbamate (E-128d): Compound E-128d (0.4 g) was synthesized by following procedure as described for the synthesis of E-34 (step-2) using tert-butyl (4-((7-((6-chloropyridazin-3-yl)oxy)-2-oxospiro[benzo[e][1,3]oxazine-4,1′-cyclobutan]-3(2H)-yl)methyl)-3-fluoropyridin-2-yl)carbamate (E-128c) (0.8 g, 1.48 mmol) as the starting material. LCMS (ES) m/z: 408.1 (M-Boc)+H]⁺.

Step-5: Synthesis of 3-((2-amino-3-fluoropyridin-4-yl)methyl)-7-(pyridazin-3-yloxy)spiro[benzo[e][1,3]oxazine-4,1′-cyclobutan]-2(3H)-one (E-128e): Compound E-128e (0.250 g) was synthesized by following procedure as described for the synthesis of E-34 (step-3) using tert-butyl-3-fluoro-4-({2-oxo-7-(3-pyridazinyloxy)-2H,3H-spiro[1,3-benzoxazine-4,1′-cyclobutan]-3-yl}methyl)-2-pyridylaminoformylate (E-128d) (0.4 g, 788 μmol) as the starting material. LCMS (ES) m/z: 408.1 [M+H]⁺.

Step-6: Synthesis of 3-{[3-fluoro-2-(methylaminosulfonylamino)-4-pyridyl]methyl}-7-(3-pyridazinyloxy)-2H,3H-spiro[1,3-benzoxazine-4,1′-cyclobutan]-2-one (E-128): Example 128 (0.022 g) was synthesized by following procedure as described for the synthesis of E-17 (step-4) using 3-fluoro-4-({2-oxo-7-(3-pyridazinyloxy)-2H,3H-spiro[1,3-benzoxazine-4,1′-cyclobutan]-3-yl}methyl)-2-pyridylamine (E-128e) (0.2 g, 491 μmol) as the starting material. The final compound was purified by reverse phase preparative HPLC purification [using Sunfire-C18 (19 mm×250 mm, 5 μm particle size) column, water and acetonitrile as mobile phase with 19.0 mL/min flow rate]. LCMS (ES) m/z: 501.1 [M+H]⁺.

¹H NMR (400 MHz, DMSO-d₆): δ 10.39 (s, 1H); 9.05 (dd, J₁=1.2 Hz, J₂=4.4 Hz, 1H); 8.00 (d, J=5.2 Hz, 1H); 7.82-7.78 (m, 2H); 7.54 (dd, J₁=1.2 Hz, J₂=9.2 Hz, 1H); 7.18 (dd, J₁=2.4 Hz, J₂=8.4 Hz, 1H); 7.07 (d, J=2.4 Hz, 1H); 7.00-6.94 (m, 1H); 6.77 (t, J=4.8 Hz, 1H); 5.02 (s, 2H); 2.88-2.78 (m, 2H); 2.51 (d, J=4.8 Hz, 3H); 2.44-2.38 (m, 2H); 2.02-1.98 (m, 2H).

### Example 129: Preparation of 6-fluoro-3-{[3-fluoro-2-(methylaminosulfonylamino)-4-pyridyl]methyl}-7-(3-pyridazinyloxy)-2H,3H-spiro[1,3-benzoxazine-4,1′-cyclobutan]-2-one (E-129; Compound 2)

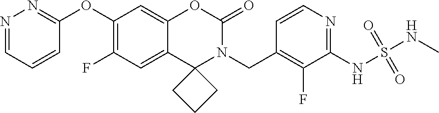 [SMILES: CNS(=O)(=O)Nc1nccc(CN2C(=O)Oc3cc(Oc4cccnn4)c(F)cc3C23CCC3)c1F]

E-129 was synthesized by following similar synthesis sequence and experimental procedures as described for the synthesis of E-128, using key intermediates I-41 and I-35. LCMS (ES) m/z: 519.3 [M+H]⁺. ¹H NMR (400 MHz, DMSO d₆): (10.43 (s, 1H); 9.07 (dd, J₁=1.2 Hz, J₂=4.8 Hz, 1H); 7.96 (s, 1H); 7.86-7.80 (m, 2H); 7.66 (dd, J₁=1.2 Hz, J₂=8.8 Hz, 1H); 7.35 (d, J=6.4 Hz, 1H); 7.00-6.62 (m, 2H); 5.00 (s, 2H); 2.84-2.78 (m, 2H); 2.55-2.49 (m, 2H), 2.45 (d, J=4.4 Hz, 3H); 2.10-1.96 (m, 2H).

### Example 130: Preparation of 3-{[3-fluoro-2-(methylaminosulfonylamino)-4-pyridyl]methyl}-7-(1,2,4-triazin-3-yloxy)-2H,3H-spiro[1,3-benzoxazine-4,1′-cyclobutan]-2-one (E-130; Compound 9)

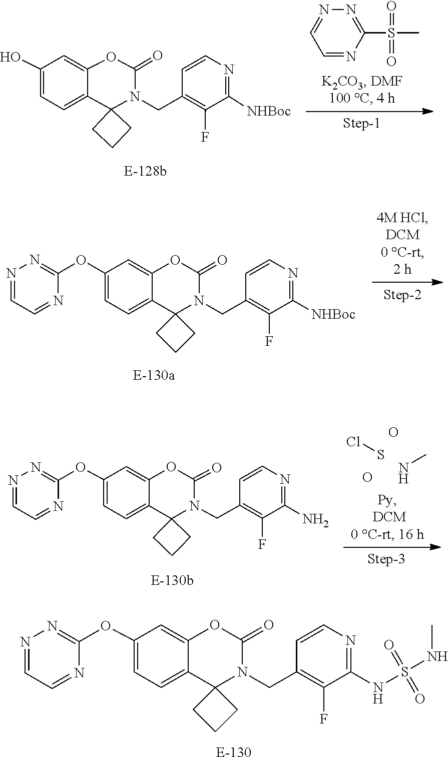

Step-1: Synthesis of tert-butyl (2-fluoro-3-(((2-hydroxy-4-((4-methoxybenzyl)oxy) benzyl)amino)methyl)phenyl)carbamate (E-130a): Compound E-130a (0.430 g) was synthesized by following procedure as described for the synthesis of E-127 (step-1) using tert-butyl-3-fluoro-4-{(7-hydroxy-2-oxo-2H,3H-spiro[1,3-benzoxazine-4,1′-cyclobutan]-3-yl)methyl}-2-pyridylaminoformylate (E-128b) (0.7 g, 1.63 mmol) and 3-(methylsulfonyl)-1,2,4-triazine as the starting materials. LCMS (ES) m/z: 509.1 [M+H]⁺.

Step-2: Synthesis of tert-butyl (2-fluoro-3-(((2-hydroxy-4-((4-methoxybenzyl)oxy) benzyl)amino)methyl)phenyl)carbamate (E-130b): Compound E-130b (0.330 g) was synthesized by following procedure as described for the synthesis of E-34 (step-3) using 3-(3-{[2-(tert-butoxycarbonylamino)-3-fluoro-4-pyridyl]methyl}-2-oxo-2H,3H-spiro[1,3-benzoxazine-4,1′-cyclobutan]-7-yloxy)-1,2,4-triazine (E-130a) (0.43 g, 0.84 mmol) as the starting material. LCMS (ES) m/z: 409.1 [M+H]⁺.

Step-3: Synthesis of tert-butyl (2-fluoro-3-(((2-hydroxy-4-((4-methoxybenzyl)oxy) benzyl)amino)methyl)phenyl)carbamate (E-130): Example 130 (0.02 g) was synthesized by following procedure as described for the synthesis of E-17 (step-4) using 3-fluoro-4-({2-oxo-7-(1,2,4-triazin-3-yloxy)-2H,3H-spiro[1,3-benzoxazine-4,1′-cyclobutan]-3-yl}methyl)-2-pyridylamine (E-130b) (0.33 g, 0.80 mmol) as the starting material. The final compound was purified by reverse phase preparative HPLC purification [using Sunfire-C18 (19 mm×250 mm, 5 m particle size) column, water and acetonitrile as mobile phase with 19.0 mL/min flow rate]. LCMS (ES) m/z: 502.3 [M+H]⁺. ¹H NMR (400 MHz, DMSO-d₆): δ 10.39 (s, 1H); 9.30 (d, J=2.4 Hz, 1H); 8.77 (d, J=2.4 Hz, 1H); 7.96 (d, J=4.8 Hz, 1H); 7.83 (d, J=8.8 Hz, 1H); 7.27 (dd, J₁=2.0 Hz, J₂=8.8 Hz, 1H); 7.18 (d, J=2.4 Hz, 1H); 6.91 (br s, 1H); 6.72 (s, 1H); 5.01 (s, 2H); 2.88-2.78 (m, 2H); 2.54 (d, J=4.4 Hz, 3H); 2.44-2.32 (m, 2H); 2.02-1.96 (m, 2H).

### Example 131: Preparation of 3-{[3-fluoro-2-(methylaminosulfonylamino)-4-pyridyl]methyl}-7-(1,3-oxazol-2-yloxy)-2H,3H-spiro[1,3-benzoxazine-4,1′-cyclobutan]-2-one (E-131; Compound 8)

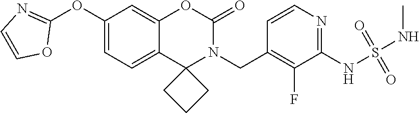 [SMILES: CNS(=O)(=O)Nc1nccc(CN2C(=O)Oc3cc(Oc4ncco4)ccc3C23CCC3)c1F]

E-131 was synthesized by following similar synthesis sequence and experimental procedures as described for the synthesis of E-130, using key intermediates I-39 and I-35. LCMS (ES) m/z: 490.3 [M+H]⁺. ¹H NMR (400 MHz, DMSO-d₆): δ 10.39 (s, 1H); 7.97 (d, J=4.4 Hz, 1H); 7.86-7.81 (m, 2H); 7.33 (dd, J₁=2.4 Hz, J₂=8.8 Hz, 1H); 7.26 (d, J=2.4 Hz, 1H); 7.06 (d, J=1.2 Hz, 1H); 6.98 (s, 1H); 6.73 (s, 1H); 5.01 (s, 2H); 2.85-2.76 (m, 2H); 2.54 (d, J=4.4 Hz, 3H); 2.40-2.30 (m, 2H); 2.02-1.94 (m, 2H).

### Example 132: Preparation of 3-({2-fluoro-3-[(methylsulfamoyl)methyl]phenyl}methyl)-2-oxo-2,3-dihydrospiro[1,3-benzoxazine-4,3′-oxetan]-7-yl N,N-dimethylcarbamate (E-132; Compound 47)

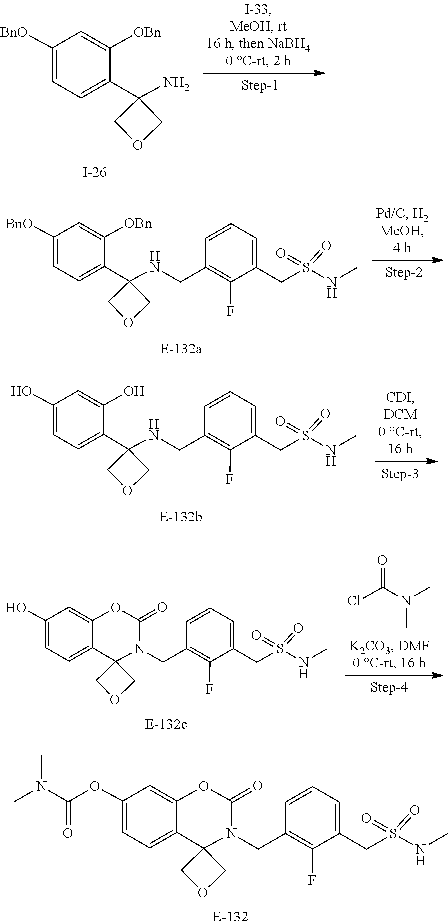

Step-1: Synthesis of 1-{3-[({3-[2,4-bis(benzyloxy)phenyl]oxetan-3-yl}amino)methyl]-2-fluorophenyl}-N-methylmethanesulfonamide (E-132a): Compound E-132a (3.0 g) was synthesized by following procedure as described for the synthesis of E-01 (step-1) using (1-(2-fluoro-3-formylphenyl)-N-methylmethanesulfonamide (I-33) (1.43 g, 6.23 mmol) and (3-[2,4-bis(benzyloxy)phenyl]oxetan-3-amine (1-26) (2.8 g, 7.75 mmol) as the starting materials. LCMS (ES) m/z: 577.0 [M+H]⁺.

Step-2: Synthesis of 1-[3-({[3-(2,4-dihydroxyphenyl)oxetan-3-yl]amino}methyl)-2-fluorophenyl]-N-methylmethanesulfonamide (E-132b): Compound E-132b (1.8 g) was synthesized by following procedure as described for the synthesis of E-55 (step-2) using 1-{3-[({3-[2,4-bis(benzyloxy)phenyl]oxetan-3-yl}amino)methyl]-2-fluorophenyl}-N-methylmethanesulfonamide (E-132a) (3.0 g, 5.2 mmol) as the starting material. LCMS (ES) m/z: 397.1 [M+H]⁺.

Step-3: Synthesis of 1-[2-fluoro-3-({7-hydroxy-2-oxo-2,3-dihydrospiro[1,3-benzoxazine-4,3′-oxetan]-3-yl}methyl)phenyl]-N-methylmethanesulfonamide (E-132c): Compound E-132c (0.5 g) was synthesized by following procedure as described for the synthesis of E-01 (step-2) using 1-[3-({[3-(2,4-dihydroxyphenyl)oxetan-3-yl]amino}methyl)-2-fluorophenyl]-N-methylmethanesulfonamide (E-132b) (0.9 g, 2.27 mmol) as the starting material. LCMS (ES) m/z: 423.0 [M+H]⁺.

Step-4: Synthesis of 3-({2-fluoro-3-[(methylsulfamoyl)methyl]phenyl}methyl)-2-oxo-2,3-dihydrospiro[1,3-benzoxazine-4,3′-oxetan]-7-yl N,N-dimethylcarbamate (E-132): Example 132 (0.088 g) was synthesized by following procedure as described for the synthesis of E-01 (step-4) 1-[2-fluoro-3-({7-hydroxy-2-oxo-2,3-dihydrospiro[1,3-benzoxazine-4,3′-oxetan]-3-yl}methyl)phenyl]-N-methylmethanesulfonamide (E-132c) (0.25 g, 0.59 mmol) as the starting material. The final compound was purified by preparative HPLC purification [using X-Select CSH C18 (19 mm×250 mm, 5 μm particle size) column, 0.1% ammonia in water and acetonitrile as mobile phase with 18.0 mL/min flow rate]. LCMS (ES) m/z: 494.3 [M+H]⁺. ¹H NMR (400 MHz, DMSO-d₆): 37.90 (d, J=8.4 Hz, 1H); 7.35 (t, J=6.8 Hz, 1H); 7.22-7.10 (m, 4H); 6.92 (d, J=2.4 Hz, 1H); 5.16 (s, 2H); 5.06 (d, J=8.0 Hz, 2H); 4.78 (d, J=8.0 Hz, 2H), 4.41 (s, 2H); 3.00 (s, 3H); 2.92 (s, 3H); 2.55 (d, J=5.2 Hz, 3H).

### Example 133: Preparation of 1-(2-fluoro-3-{[2-oxo-7-(pyridazin-3-yloxy)-2,3-dihydrospiro[1,3-benzoxazine-4,3′-oxetan]-3-yl]methyl}phenyl)-N-methylmethanesulfonamide (E-133; Compound 46)

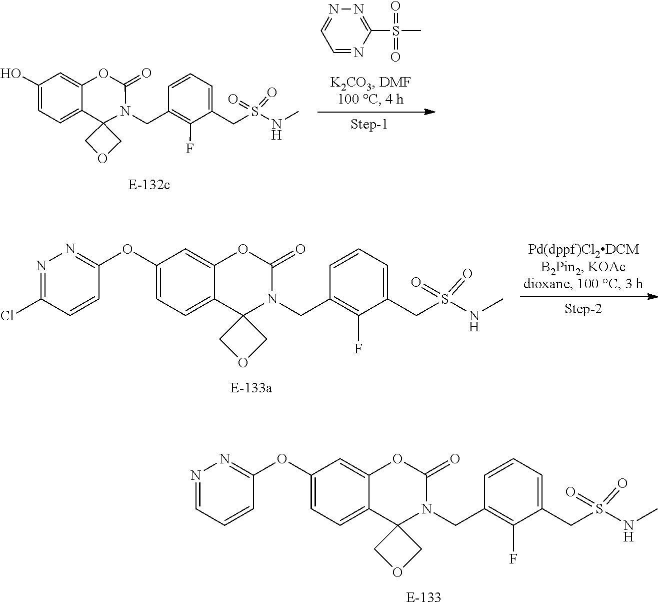

Step-1: Synthesis of 1-[3-({7-[(6-chloropyridazin-3-yl)oxy]-2-oxo-2,3-dihydrospiro[1,3-benzoxazine-4,3′-oxetan]-3-yl}methyl)-2-fluorophenyl]-N-methylmethanesulfonamide (E-133a): Compound E-133a (0.42 g) was synthesized by following procedure as described for the synthesis of E-34 (step-1) using 1-[2-fluoro-3-({7-hydroxy-2-oxo-2,3-dihydrospiro[1,3-benzoxazine-4,3′-oxetan]-3-yl}methyl)phenyl]-N-methylmethanesulfonamide (E-132c) (0.7 g, 1.66 mmol) as the starting material. LCMS (ES) m/z: 535.1 [M+H]⁺.

Step-2: Synthesis of 1-(2-fluoro-3-{[2-oxo-7-(pyridazin-3-yloxy)-2,3-dihydrospiro[1,3-benzoxazine-4,3′-oxetan]-3-yl]methyl}phenyl)-N-methylmethanesulfonamide (E-133): Example 133 (0.087 g) was synthesized by following procedure as described for the synthesis of E-34 (step-2) using 1-[3-({7-[(6-chloropyridazin-3-yl)oxy]-2-oxo-2,3-dihydrospiro[1,3-benzoxazine-4,3′-oxetan]-3-yl}methyl)-2-fluorophenyl]-N-methylmethanesulfonamide (E-133a) (370 mg, 692 μmol) as the starting material. The final compound was purified by preparative HPLC purification [using Sunfire C18 (250 mm×19 mm, 5 μm particle size) column, 10 mM ammonium acetate in water and acetonitrile:methanol (1:1) as mobile phase with 19.0 mL/min flow rate]. LCMS (ES) m/z: 501.3 [M+H]⁺. ¹H NMR (400 MHz, DMSO-d₆): δ 9.05 (dd, J₁=1.2 Hz, J₂=4.8 Hz, 1H); 7.99 (d, J=8.4 Hz, 1H); 7.84-7.79 (m, 1H); 7.54 (dd, J₁=1.2 Hz, J₂=9.2 Hz, 1H); 7.35 (t, J=6.8 Hz, 1H); 7.24-7.10 (m, 4H); 7.06 (d, J=2.4 Hz, 1H); 5.13 (s, 2H); 5.09 (d, J=8.0 Hz, 2H); 4.82 (d, J=7.6 Hz, 2H); 4.41 (s, 2H); 2.62 (d, J=4.4 Hz, 3H).

### Example 134: Preparation of (cyclopropylsulfonyl)[3-fluoro-4-({6-fluoro-2-oxo-7-(3-pyridazinyloxy)-2H,3H-spiro[1,3-benzoxazine-4,3′-oxetan]-3-yl}methyl)-2-pyridyl]amine (E-134; Compound 5)

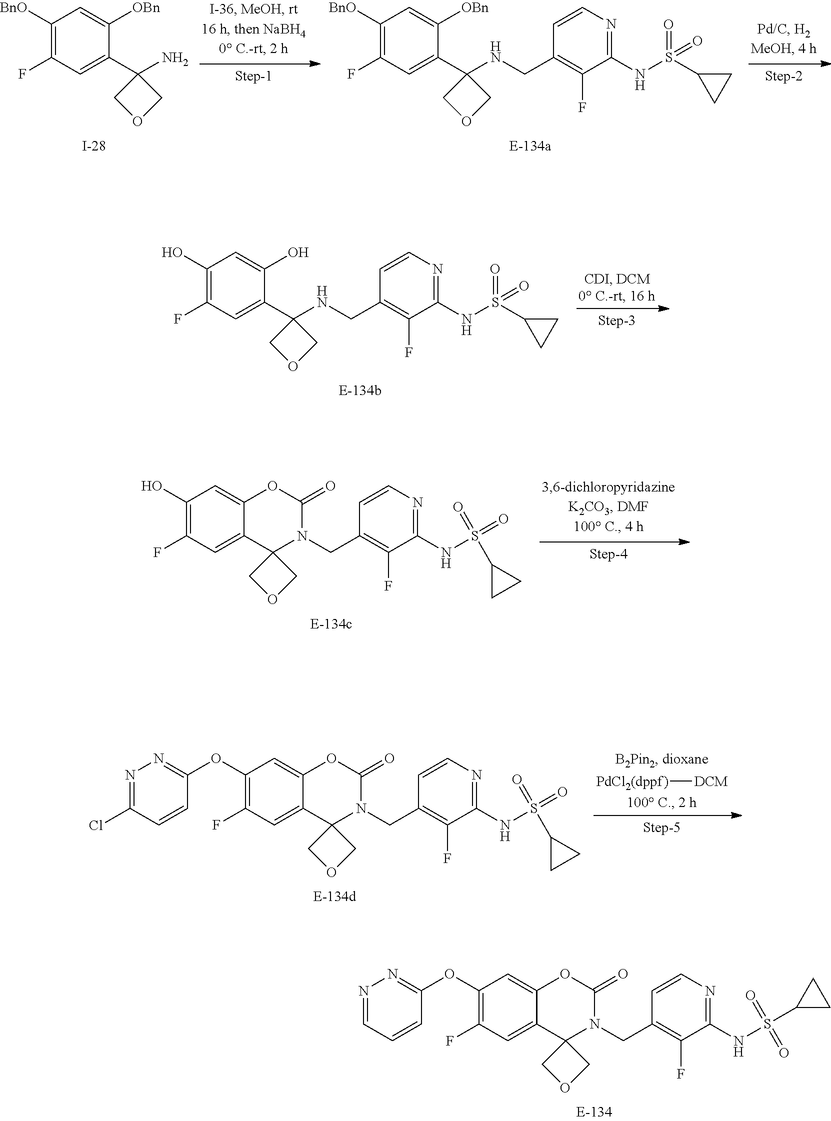

Step-1: Synthesis of 3-[2,4-bis(benzyloxy)-5-fluorophenyl]-3-({[2-(cyclopropylsulfonylamino)-3-fluoro-4-pyridyl]methyl}amino)oxetane (134a): Compound E-134a (1.2 g) was synthesized by following procedure as described for the synthesis of E-01 (step-1) using 3-[2,4-bis(benzyloxy)-5-fluorophenyl]-3-oxetanylamine (I-28) (0.9 g, 2.37 mmol) and 2-(cyclopropylsulfonylamino)-3-fluoroisonicotinaldehyde (579 mg, 1.0 eq., 2.37 mmol) as the starting materials. LCMS (ES) m/z: 608.1 [M+H]⁺.

Step-2: Synthesis of 4-[3-({[2-(cyclopropylsulfonylamino)-3-fluoro-4-pyridyl]methyl}amino)-3-oxetanyl]-6-fluororesorcinol (134b): Compound E-134b (0.6 g) was synthesized by following procedure as described for the synthesis of E-97 (step-2) using 3-[2,4-bis(benzyloxy)-5-fluorophenyl]-3-({[2-(cyclopropylsulfonylamino)-3-fluoro-4-pyridyl]methyl}amino)oxetane (E-134a) (1.2 g, 1.97 mmol) as the starting material. LCMS (ES) m/z: 428.1 [M+H]⁺.

Step-3: Synthesis of 3-{[2-(cyclopropylsulfonylamino)-3-fluoro-4-pyridyl]methyl}-6-fluoro-7-hydroxy-2H,3H-spiro[1,3-benzoxazine-4,3′-oxetan]-2-one (134c): Compound E-134c (0.3 g) was synthesized by following procedure as described for the synthesis of E-01 (step-2) using 4-[3-({[2-(cyclopropylsulfonylamino)-3-fluoro-4-pyridyl]methyl}amino)-3-oxetanyl]-6-fluororesorcinol (E-134b) (0.6 g, 1.4 mmol) as the starting material. LCMS (ES) m/z: 454.1 [M+H]⁺.

Step-4: Synthesis of (cyclopropylsulfonyl)[4-({7-(6-chloro-3-pyridazinyloxy)-6-fluoro-2-oxo-2H,3H-spiro[1,3-benzoxazine-4,3′-oxetan]-3-yl}methyl)-3-fluoro-2-pyridyl]amine (134d): Compound E-134d (0.280 g) was synthesized by following procedure as described for the synthesis of E-34 (step-1) using 3-{[2-(cyclopropylsulfonylamino)-3-fluoro-4-pyridyl]methyl}-6-fluoro-7-hydroxy-2H,3H-spiro[1,3-benzoxazine-4,3′-oxetan]-2-one (134c) (0.3 g, 662 μmol) and 3,6-dichloropyridazine (197 mg, 1.32 mmol) as the starting materials. LCMS (ES) m/z: 566.2 [M+H]⁺.

Step-5: Synthesis of (cyclopropylsulfonyl)[3-fluoro-4-({6-fluoro-2-oxo-7-(3-pyridazinyloxy)-2H,3H-spiro[1,3-benzoxazine-4,3′-oxetan]-3-yl}methyl)-2-pyridyl]amine (E-134): Example 134 (0.018 g) was synthesized by following procedure as described for the synthesis of E-34 (step-2) using (cyclopropylsulfonyl)[4-({7-(6-chloro-3-pyridazinyloxy)-6-fluoro-2-oxo-2H,3H-spiro[1,3-benzoxazine-4,3′-oxetan]-3-yl}methyl)-3-fluoro-2-pyridyl]amine (E-134d) (280 mg, 495 μmol) as the starting material. The final compound was purified by reverse phase preparative HPLC purification, using Atlantis C18 (100 mm×190 mm, 5 μm particle size) column, 5 mM ammonium acetate in water and acetonitrile as mobile phase with 19 mL/min flow. LCMS (ES) m/z: 532.2 [M+H]⁺. ¹H NMR (400 MHz, DMSO-d₆): δ 10.62 (s, 1H); 9.07 (dd, J₁=1.2 Hz, J₂=4.8 Hz, 1H); 8.04-7.96 (m, 2H); 7.88-7.82 (m, 1H); 7.66 (dd, J₁=1.2 Hz, J₂=8.8 Hz 1H); 7.32 (d, J=6.4 Hz, 1H); 7.09 (br s, 1H); 5.12 (s, 2H); 5.08 (d, J=8.0 Hz, 2H); 4.88 (d, J=8.0 Hz, 2H); 3.22-3.16 (m, 1H); 1.12-0.98 (m, 4H).

### Examples 135 and 136: Preparations of (S)-3-{[3-fluoro-2-(methylaminosulfonylamino)-4-pyridyl]methyl}-4-methyl-7-(3-pyridazinyloxy)-3,4-dihydro-2H-1,3-benzoxazin-2-one and (R)-3-{[3-fluoro-2-(methylaminosulfonylamino)-4-pyridyl]methyl}-4-methyl-7-(3-pyridazinyloxy)-3,4-dihydro-2H-1,3-benzoxazin-2-one (E-135 and E-136; Compounds 22 and 23, respectively)

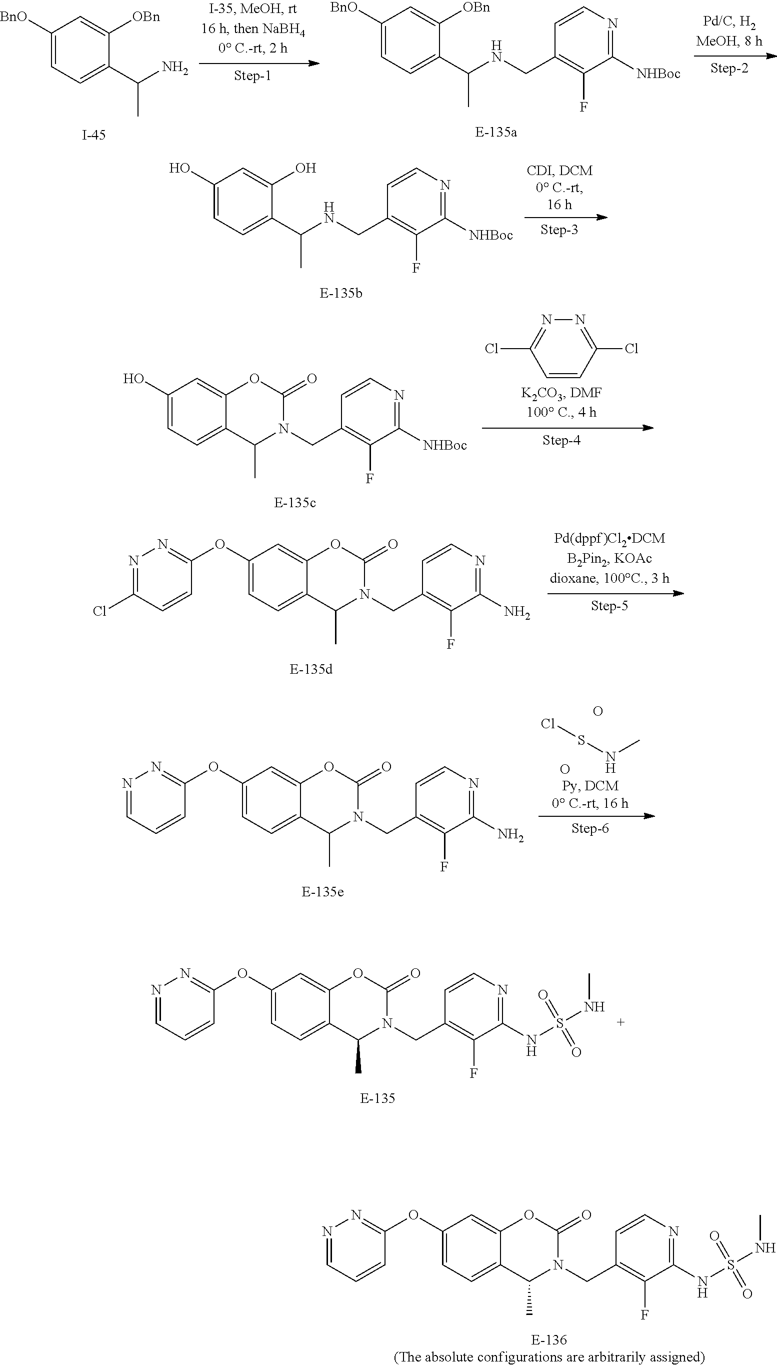

Step-1: Synthesis of tert-butyl N-{4-[(1-[2,4-bis(benzyloxy)phenyl]ethyl}amino)methyl]-3-fluoropyridin-2-yl}carbamate (E-135a): Compound E-135a (2.7 g) was synthesized by following procedure as described for the synthesis of E-01 (step-1) using 1-12,4-bis(benzyloxy)phenyllethan-1-amine) (1.75 g, 5.25 mmol) and tert-butyl N-(3-fluoro-4-formylpyridin-2-yl)carbamate (I-35) (1.26 g, 5.25 mmol) as the starting materials. LCMS (ES) m/z: 558.2 [M+H]⁺.

Step-2: Synthesis of tert-butyl N-[4-({[1-(2,4-dihydroxyphenyl)ethyl]amino}methyl)-3-fluoropyridin-2-yl]carbamate (E-135b): Compound E-135b (1.8 g) was synthesized by following procedure as described for the synthesis of E-97 (step-2) using tert-butyl N-{4-1({1-12,4-bis(benzyloxy)phenyl]ethyl Iamino)methyl1-3-fluoropyridin-2-yl}carbamate (E-135a) (2.7 g, 4.84 mmol) as the starting material. LCMS (ES) m/z: 378.2 [M+H]⁺.

Step-3: Synthesis of tert-butyl N-{3-fluoro-4-[(7-hydroxy-4-methyl-2-oxo-3,4-dihydro-2H-1,3-benzoxazin-3-yl)methyl]pyridin-2-yl}carbamate (E-135c): Compound E-135c (0.92 g) was synthesized by following procedure as described for the synthesis of E-01 (step-2) using tert-butyl N-14-({1-(2,4-dihydroxyphenyl)ethyl|amino}methyl)-3-fluoropyridin-2-yl]carbamate (E-135b) (1.85 g, 4.90 mmol) as the starting material. LCMS (ES) m/z: 404.1 [M+H]⁺.

Step-4: Synthesis of 4-f[7-(6-chloro-3-pyridazinyloxy)-4-methyl-2-oxo-3,4-dihydro-2H-1,3-benzoxazin-3-yl]methyl}-3-fluoro-2-pyridylamine (E-135d): Compound E-135d (0.70 g) was synthesized by following procedure as described for the synthesis of E-34 (step-1) using tert-butyl N-{3-fluoro-4-[(7-hydroxy-4-methyl-2-oxo-3,4-dihydro-2H-1,3-benzoxazin-3-yl)methyl]pyridin-2-yl}carbamate (E-135c) (0.92 g, 2.28 mmol) and 3,6-dichloropyridazine (11) (0.679 g, 4.56 mmol) as the starting materials. LCMS (ES) m/z: 416.1 [M+H]⁺.

Step-5: Synthesis of 3-fluoro-4-{[4-methyl-2-oxo-7-(3-pyridazinyloxy)-3,4-dihydro-2H-1,3-benzoxazin-3-yl]methyl}-2-pyridylamine (E-135e): To a stirred solution of 4-{[7-(6-chloro-3-pyridazinyloxy)-4-methyl-2-oxo-3,4-dihydro-2H-1,3-benzoxazin-3-yl]methyl}-3-fluoro-2-pyridylamine (E-135d) (0.7 g, 1.68 mmol) in 1,4-dioxane (9 mL) was added KOAc (0.496 g, 5.05 mmol) and bis-pinacolato diboron (0.47 g, 1.85 mmol). Argon gas was purged through it for 5 min before addition of PdCl₂(dppf). DCM (0.137 g, 0.168 mmol). The reaction mixture was then stirred at 100° C., while monitoring reaction progress by TLC/LCMS analysis. After complete consumption of starting material (3 h), the reaction mixture was cooled to room temperature and water (15 mL) was added to it. Extraction was carried out using EtOAc (30 mL×3); the combined organic extracts were washed with water (50 mL), brine (50 mL), dried over anhydrous Na₂SO₄, filtered and concentrated under reduced pressure. The residue was purified by Combi-Flash (using gradient elution of 0-10% of MeOH in DCM) to afford desired compound E-135e (0.72 g) as a brown semi-solid. LCMS (ES) m/z: 382.1 [M+H]⁺.

Step-6: Synthesis of 3-{[3-fluoro-2-(methylaminosulfonylamino)-4-pyridyl]methyl}-4-methyl-7-(3-pyridazinyloxy)-3,4-dihydro-2H-1,3-benzoxazin-2-one (E-135): Example E-135 (34 mg) and E-136 (33 mg) were synthesized by following procedure as described for the synthesis of E-17 (step-4) using 3-fluoro-4-{[4-methyl-2-oxo-7-(3-pyridazinyloxy)-3,4-dihydro-2H-1,3-benzoxazin-3-yl]methyl}-2-pyridylamine (E-135e) (0.72 g, 1.89 mmol) as the starting material. The final compound was purified by preparative HPLC purification [using Eclipse C18 (150 mm×21 mm, 7 μm particle size) column, 0.1% TFA in water and acetonitrile as mobile phase with 19 mL/min flow rate] followed by resolution using SFC purification. Note: Absolute configuration of both the enantiomers was not determined.

Example-135: LCMS (ES) m/z: 475.20 [M+H]⁺. ¹H NMR (400 MHz, DMSO-dh): δ 10.42 (s, 1H); 9.03 (d, J=4.4 Hz, 1H); 8.04 (d, J=5.2 Hz, 1H); 7.82-7.77 (m, 1H); 7.51 (d, J=8.8 Hz, 1H); 7.39 (d, J=8.0 Hz, 1H); 7.09-7.02 (m, 2H); 6.96-6.93 (m, 1H); 4.91 (d, J=16.4 Hz, 1H); 4.74 (q, J=6.0 Hz, 1H); 4.57 (d, J=16.4 Hz, 1H); 2.50 (d, J=4.8 Hz, 3H); 1.46 (d, J=6.4 Hz, 3H).

Example-136: LCMS (ES) m/z: 475.20 [M+H]⁺. ¹H NMR (400 MHz, DMSO-d₆): δ 10.43 (br s, 1H); 9.03 (d, J=4.4 Hz, 1H); 8.01 (br s.1H); 7.82-7.77 (m, 1H); 7.51 (d, J=8.4 Hz, 1H); 7.39 (d, J=8.0 Hz, 1H); 7.09-6.87 (m, 3H); 4.90 (d, J=16.4 Hz, 1H); 4.73 (q, J=7.6 Hz, 1H); 4.56 (d, J=16.4 Hz, 1H); 2.50 (d, J=4.4 Hz, 3H); 1.46 (d, J=6.0 Hz, 3H).

### Example 137: Preparation of (R)-6-fluoro-3-{[3-fluoro-2-(methylaminosulfonylamino)-4-pyridyl]methyl}-4-methyl-7-(3-pyridazinyloxy)-3,4-dihydro-2H-1,3-benzoxazin-2-one or (S)-6-fluoro-3-{[3-fluoro-2-(methylaminosulfonylamino)-4-pyridyl]methyl}-4-methyl-7-(3-pyridazinyloxy)-3,4-dihydro-2H-1,3-benzoxazin-2-one (E-137; Compound 13)

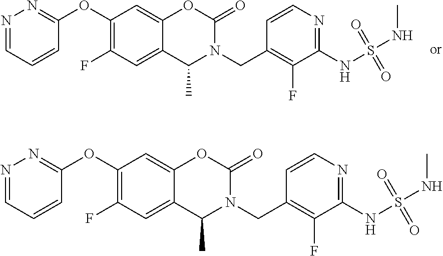 [SMILES: CNS(=O)(=O)Nc1nccc(CN2C(=O)Oc3cc(Oc4cccnn4)c(F)cc3[C@H]2C)c1F ; CNS(=O)(=O)Nc1nccc(CN2C(=O)Oc3cc(Oc4cccnn4)c(F)cc3[C@@H]2C)c1F]

E-137 was synthesized by following similar synthesis sequence and experimental procedures as described for the synthesis of E-135, using key intermediates I-53 and I-35. LCMS (ES) m/z: 493.27 [M+H]⁺. ¹H NMR, (400 MHz, DMSO-d₆): δ 10.38 (s, 1H); 9.05 (dd, J₁=1.2 Hz, J₂=4.4 Hz, 1H); 8.05 (s, 1H); 7.85-7.81 (m, 1H); 7.63 (dd, J₁=1.2 Hz, J₂=8.8 Hz, 1H); 7.49 (d, J=10.4 Hz, 1H); 7.32 (d, J=6.8 Hz, 1H); 6.96 (br s, 2H); 4.91 (d, J=16.4 Hz, 1H); 4.73 (q, J=6.4 Hz, 1H); 4.57 (d, J=16.4 Hz, 1H); 2.49 (d, J=4.4 Hz, 3H); 1.49 (d, J=6.4 Hz, 3H). Note: Absolute configuration was not determined.

### Example 138: Preparation of (S)-6-fluoro-3-{[3-fluoro-2-(methylaminosulfonylamino)-4-pyridyl]methyl}-4-methyl-7-(3-pyridazinyloxy)-3,4-dihydro-2H-1,3-benzoxazin-2-one or (R)-6-fluoro-3-{[3-fluoro-2-(methylaminosulfonylamino)-4-pyridyl]methyl}-4-methyl-7-(3-pyridazinyloxy)-3,4-dihydro-2H-1,3-benzoxazin-2-one (E-138; Compound 12)

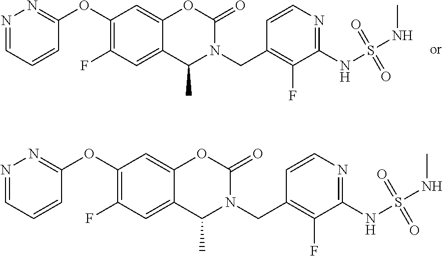 [SMILES: CNS(=O)(=O)Nc1nccc(CN2C(=O)Oc3cc(Oc4cccnn4)c(F)cc3[C@@H]2C)c1F ; CNS(=O)(=O)Nc1nccc(CN2C(=O)Oc3cc(Oc4cccnn4)c(F)cc3[C@H]2C)c1F]

E-138 was synthesized by following similar synthesis sequence and experimental procedures as described for the synthesis of E-135, using key intermediates I-53 and I-35. LCMS (ES) m/z: 493.27[M+H]⁺. ¹H NMR (400 MHz, DMSO-d₆): δ 10.38 (s, 1H); 9.05 (dd, J₁=1.2 Hz, J₂=4.4 Hz, 1H); 8.04 (br s, 1H); 7.85-7.81 (m, 1H); 7.63 (dd, J₁=1.2 Hz, J₂=9.2 Hz, 1H); 7.49 (d, J=10.4 Hz, 1H); 7.32 (d, J=6.8 Hz, 1H); 6.98 (br s, 2H); 4.90 (d, J=16.4 Hz, 1H); 4.72 (q, J=6.8 Hz, 1H); 4.57 (d, J=16.4 Hz, 1H); 2.49 (d, J=4.4 Hz, 3H); 1.49 (d, J=6.4 Hz, 3H). Note: Absolute configuration was not determined.

### Examples 139 and 140: Preparations of (S)-3-({3-(methylsulfamoyl)amino]phenyl}methyl)-7-(pyrimidin-2-yloxy)-3,4-dihydro-2H-1,3-benzoxazin-2-one and I-3-({3-[(methylsulfamoyl)amino]phenyl}methyl)-7-(pyrimidin-2-yloxy)-3,4-dihydro-2H-1,3-benzoxazin-2-one and (R)-3-({3-(methylsulfamoyl)amino]phenyl}methyl)-7-(pyrimidin-2-yloxy)-3,4-dihydro-2H-1,3-benzoxazin-2-one (E-139 and E-140; Compounds 30 and 29, respectively)

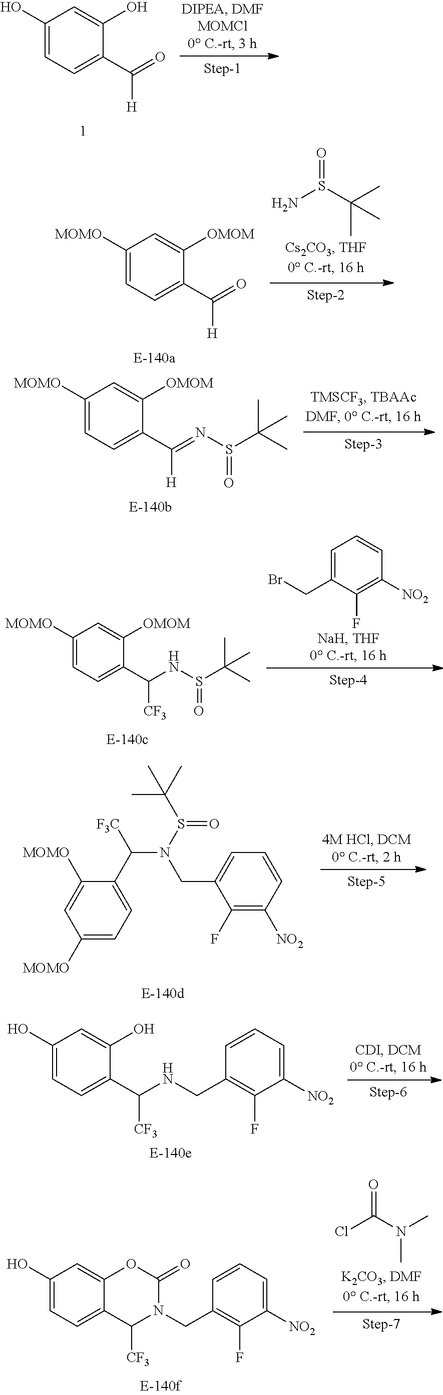

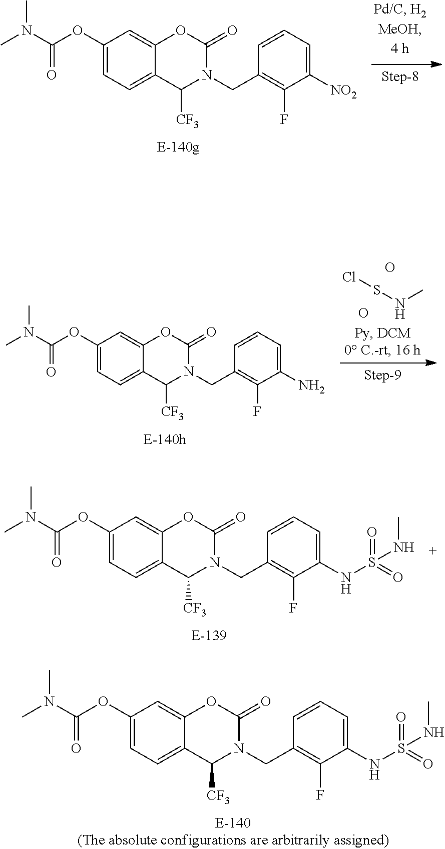

Step-1: Synthesis of 2,4-bis(methoxymethoxy)benzaldehyde (E-140a): To a stirred solution of 2,4-dihydroxybenzaldehyde (1) (3 g, 21.7 mmol) in DMF (30 mL) was added DIPEA (18.5 mL, 106 mmol) at 0° C. and the reaction mixture was stirred for 10 min. To this was then added MOM-C₁ (6.03 mL, 79.04 mmol) and reaction mixture was allowed to stir at room temperature. The reaction progress was monitored by TLC/LCMS analysis. After completion of the reaction (3 h), water (60 mL) was added to it and extraction was carried out using EtOAc (30 mL×3). The combined organic extracts were washed with water (50 mL), brine (50 mL), dried over anhydrous Na₂SO₄, filtered and concentrated under reduced pressure. The residue was purified by Combi-Flash (using gradient elution of 0-10% of EtOAc in heptane) to afford desired compound E-140a (4.2 g) as an off white solid. LCMS (ES) n/z: 227.1 [M+H]⁺.

Step-2: Synthesis I(E)-N-(2,4-bis(methoxymethoxy)benzylidene)-2-methylpropane-2-sulfinamide (E-140b): To a stirred solution of 2,4-bis(methoxymethoxy)benzaldehyde (E-140a) (3.8 g, 16.8 mmol) in THF (40 mL) was added Cs₂CO₃ (10.9 g, 33.6 mmol) and 2-methylpropane-2-sulfinamide (4.07 g, 33.6 mmol). The reaction mixture was allowed to stir at room temperature for 16 h. Cs₂CO₃ was then filtered off through celite and washed with THE (10 mL×2). THE was evaporated under reduced pressure and the residue was purified by Combi-Flash (using gradient elution of 0-25% of EtOAc in heptane) to afford desired compound E-140b as a colorless liquid (5.2 g). LCMS (ES) m/z: 330.2 [M+H]⁺.

Step-3: Synthesis of N-(1-(2,4-bis(methoxymethoxy)phenyl)-2,2,2-trifluoroethyl)-2-methylpropane-2-sulfinamide (E-140c): To a stirred solution IN-[(E)-[2,4-bis(methoxymethoxy)phenyl]methylidene]-2-methylpropane-2-sulfinamide (E-140b) (3 g, 9.11 mmol) in DMF (60 mL) was added tetrabutylammonium acetate (2.15 g, 7.12 mmol) and TMSCF₃ (2.64 mL, 17.8 mmol) at 0° C. The reaction mixture was then allowed to stir at room temperature for 16 h. After completion of the reaction (16 h), the reaction mixture was quenched with addition of ice water (50 mL) and extraction was carried out using EtOAc (2×40 mL). The combined organic extracts were washed with saturated bicarbonate solution (30 mL), brine solution (30 mL), dried over anhydrous Na₂SO₄, filtered and evaporated under reduced pressure. The residue was purified by Combi-Flash (using gradient elution of 0-30% of EtOAc in heptane) to afford desired compound E-140c as a yellow solid (1 g). LCMS (ES) m/z: 400.0 [M+H]⁺.

Step-4: Synthesis of N-(1-(2,4-bis(methoxymethoxy)phenyl)-2,2,2-trifluoroethyl)-N-(2-fluoro-3-nitrobenzyl)-2-methylpropane-2-sulfinamide (E-140d): To a stirred solution N-{1-[2,4-bis(methoxymethoxy)phenyl]-2,2,2-trifluoroethyl}-2-methylpropane-2-sulfinamide (E-140c) (1.5 g, 3.76 mmol) in THE (40 mL) was added NaH (60% dispersion in mineral oil) (0.18 g, 7.51 mmol) at 0° C. and stirred for 30 min. To this was then added a solution of 1-(bromomethyl)-2-fluoro-3-nitrobenzene (0.96 g, 4.13 mmol, in 5 mL THF) at 0° C. and reaction mixture was allowed to stir at room temperature for 16 h. After completion of the reaction, it was quenched with addition of ice water (60 mL) and extracted with EtOAc (2×40 mL). The combined organic extracts were washed with saturated bicarbonate solution (30 mL), brine solution (30 mL), dried over anhydrous Na₂SO₄, filtered and evaporated under reduced pressure The residue was purified by Combi-Flash (using gradient elution of 0-25% of EtOAc in heptane) to afford the desired product 140d as a yellow solid (0.6 g). LCMS (ES) m/z: 553.2 [M+H]⁺.

Step-5: Synthesis of 4-(2,2,2-trifluoro-1-((2-fluoro-3-nitrobenzyl)amino)ethyl)benzene-1,3-diol (E-140e): Compound E-140e (0.6 g) was synthesized by following procedure as described for the synthesis of I-26 (step-4) using N-{1-[2,4-bis(methoxymethoxy)phenyl]-2,2,2-trifluoroethyl}-N-[(2-fluoro-3-nitrophenyl)methyl]-2-methylpropane-2-sulfinamide (E-140d) (0.9 g, 1.63 mmol) as the starting material. LCMS (ES) m/z: 361.1 [M+H]⁺.

Step-6: Synthesis of 3-(2-fluoro-3-nitrobenzyl)-7-hydroxy-4-(trifluoromethyl)-3,4-dihydro-2H-benzo[e][1,3]oxazin-2-one (E-140f): Compound E-140f (0.50 g) was synthesized by following procedure as described for the synthesis of E-01 (step-2) using 4-(2,2,2-trifluoro-1-{[(2-fluoro-3-nitrophenyl)methyl]amino}ethyl)benzene-1,3-diol (E-140e) (0.6 g, 1.67 mmol) as the starting material. LCMS (ES) m/z: 387.0 [M+H]⁺.

Step-7: Synthesis of 3-(2-fluoro-3-nitrobenzyl)-2-oxo-4-(trifluoromethyl)-3,4-dihydro-2H-benzo[e][1,3]oxazin-7-yl dimethylcarbamate (E-140g): Compound E-140g (0.50 g) was synthesized by following procedure as described for the synthesis of E-01 (step-4) using 3-(2-fluoro-3-nitrobenzyl)-7-hydroxy-4-(trifluoromethyl)-3,4-dihydro-2H-benzo[e][1,3]oxazin-2-one (E-140f) (0.5 g, 1.29 mmol) as the starting material. LCMS (ES) m/z: 458.1 [M+H]⁺.

Step-8: Synthesis of 3-(3-amino-2-fluorobenzyl)-2-oxo-4-(trifluoromethyl)-3,4-dihydro-2H-benzo[e][1,3]oxazin-7-yl dimethylcarbamate (E-140h): Compound E-140h (0.38 g) was synthesized by following procedure as described for the synthesis of E-42 (step-5) using 3-[(2-fluoro-3-nitrophenyl)methyl]-2-oxo-4-(trifluoromethyl)-3,4-dihydro-2H-1,3-benzoxazin-7-yl N,N-dimethylcarbamate (E-140f) (0.5 g, 1.09 mmol) as the starting material. LCMS (ES) m/z: 428.1 [M+H]⁺.

Step-9: SynthesiIf rel-(R)-3-(2-fluoro-3-((N-methylsulfamoyl)amino)benzyl)-2-oxo-4-(trifluoromethyl)-3,4-dihydro-2H-benzo[e][1,3]oxazin-7-yl dimethylcarbamate (E-140): Example E-139 (42 mg) and E-140 (53 mg) were synthesized by following procedure as described for the synthesis of E-17 (step-4) using 3-[(3-amino-2-fluorophenyl)methyl]-2-oxo-4-(trifluoromethyl)-3,4-dihydro-2H-1,3-benzoxazin-7-yl N,N-dimethylcarbamate (E-140h) (0.38 g, 0.88 mmol) as the starting material. Both the enantiomers were separated by chiral preparative HPLC [using Chiralpak IG (250 mm×20 mm, 5 ptm particle size) column, 0.1% DEA and methanol as mobile phase with 40.0 mL/min flow rate]. Note: Absolute configuration of both the enantiomers was not determined.

Example 139: LCMS (ES) m/z: 521.3 [M+H]⁺. ¹H NMR (400 MHz, DMSO-d₆): h 9.42 (s, 1H); 7.50 (d, J=9.2 Hz, 1H); 7.37 (t, J=7.2 Hz, 1H); 7.22-7.16 (m, 1H); 7.13-7.07 (m, 3H); 6.98 (t, J=6.8 Hz, 1H); 5.83 (q, J=6.4 Hz, 1H); 5.09 (d, J=16.4 Hz, 1H); 4.54 (d, J=15.6 Hz, 1H); 3.04 (s, 3H); 2.92 (s, 3H); 2.44 (d, J=5.2 Hz, 3H).

Example 140: LCMS (ES) m/z: 521.3 [M+H]⁺. ¹H NMR (400 MHz, DMSO-d₆): δ 9.33 (s, 1H); 7.50 (d, J=9.2 Hz, 1H); 7.37 (t, J=7.2 Hz, 1H); 7.20-7.06 (m, 4H); 6.98 (t, J=6.8 Hz, 1H); 5.83 (q, J=6.8 Hz, 1H); 5.08 (d, J=15.6 Hz, 1H); 4.54 (d, J=16.0 Hz, 1H); 3.04 (s, 3H); 2.92 (s, 3H); 2.44 (d. J=4.8 Hz, 3H).

### Example 141: Preparation of 3-{[2-fluoro-3-(methylaminosulfonylamino)phenyl]methyl}-7-(3-pyridazinyloxy)-3,4-dihydro-2H-1-oxa-3,5-diazanaphthalen-2-one (E-141; Compound 15)

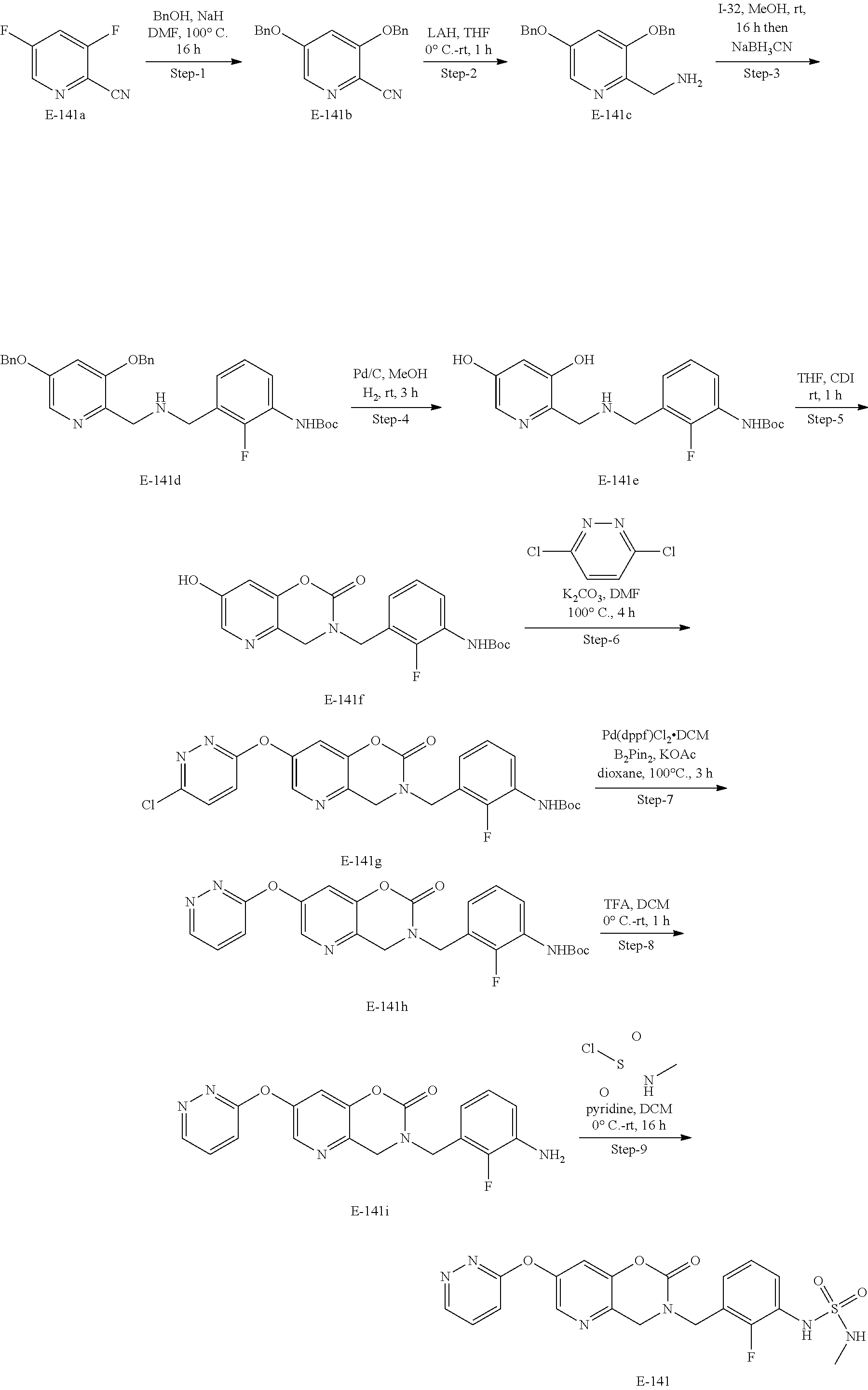

Step-1: Synthesis of 3,5-bis(benzyloxy)picolinonitrile (E-141b): E-141b (15 g) was synthesized by following procedure as described for the synthesis of E-53 (step-1) using E-141a (10 g) and benzyl alcohol as the starting materials. It was carried forward for the next step without any characterization.

Step-2: Synthesis of (3,5-bis(benzyloxy)pyridin-2-yl)methanamine (E-141c): E-141c (6 g) was synthesized by following procedure as described for the synthesis of E-53 (step-2) using E-141b (15 g) as the starting material. LCMS (ES) m/z: 321.2 [M+H]⁺.

Step-3: Synthesis of tert-butyl (3-((((3,5-bis(benzyloxy)pyridin-2-yl)methyl)amino)methyl)-2-fluorophenyl)carbamate (E-141d): E-141d (0.57 g) was synthesized by following procedure as described for the synthesis of E-01 (step-1) using E-141c (2.6 g, 8.12 mmol) and I-31 (1.55 g, 8.12 mmol) as the starting materials (sodium cyanoborohydride was used as reducing agent). LCMS (ES) m/z: 544.3 [M+H]⁺.

Step-4: Synthesis of tert-butyl (3-((((3,5-dihydroxypyridin-2-yl)methyl)amino)methyl)-2-fluorophenyl)carbamate (E-141e): E-141e (0.33 g) was synthesized by following procedure as described for the synthesis of E-42 (step-5) using E-141d (0.57 g) as the stating material. LCMS (ES) m/z: 364.2 [M+H]⁺.

Step-5: Synthesis of tert-butyl (2-fluoro-3-((7-hydroxy-2-oxo-2H-pyrido[2,3-e][1,3]oxazin-3(4H)-yl)methyl)phenyl)carbamate (E-141f): E-141f (0.13 g) was synthesized by following procedure as described for the synthesis of E-01 (step-2) using E-141e (0.33 g, 0.91 mmol) as the stating material. LCMS (ES) m/z: 390.2 [M+H]⁺.

Step-6: Synthesis of 3-{[3-(tert-butoxycarbonylamino)-2-fluorophenyl]methyl}-7-(6-chloro-3-pyridazinyloxy)-3,4-dihydro-2H-1-oxa-3,5-diazanaphthalen-2-one (E-141g): Compound E-141g (0.41 g) was synthesized by following procedure as described for the synthesis of E-34 (step-1) using tert-butyl (2-fluoro-3-((7-hydroxy-2-oxo-2H-pyrido[2,3-e][1,3]oxazin-3(4H)-yl)methyl)phenyl)carbamate (E-141f) (0.50 g, 1.28 mmol) and 3,6-dichloropyridazine (0.191 g, 1.28 mmol) as the starting materials. LCMS (ES) m/z: 500.2 [M−H]⁺.

Step-7: Synthesis of 3-{[3-(tert-butoxycarbonylamino)-2-fluorophenyl]methyl}-7-(3-pyridazinyloxy)-3,4-dihydro-2H-1-oxa-3,5-diazanaphthalen-2-one (E-141h): Compound 141h (0.24 g) was synthesized by following procedure as described for the synthesis of E-34 (step-2) using 3-{[3-(tert-butoxycarbonylamino)-2-fluorophenyl]methyl}-7-(6-chloro-3-pyridazinyloxy)-3,4-dihydro-2H-1-oxa-3,5-diazanaphthalen-2-one (E-141g) (0.40 g, 0.797 mmol) as the starting material. LCMS (ES) m/z: 466.2 [M−H]⁺.

Step-8: Synthesis of 2-fluoro-3-{[2-oxo-7-(3-pyridazinyloxy)-3,4-dihydro-2H-1-oxa-3,5-diazanaphth-3-yl]methyl}aniline (E-141i): Compound E-141i (0.170 g) was synthesized by following procedure as described for the synthesis of E-43 (step-4) using 3-{[3-(tert-butoxycarbonylamino)-2-fluorophenyl]methyl}-7-(3-pyridazinyloxy)-3,4-dihydro-2H-1-oxa-3,5-diazanaphthalen-2-one (E-141h) (0.24 g, 0.513 mmol) as the starting material. LCMS (ES) m/z: 368 [M+H]⁺.

Step-9: Synthesis of 3-{[2-fluoro-3-(methylaminosulfonylamino)phenyl]methyl}-7-(3-pyridazinyloxy)-3,4-dihydro-2H-1-oxa-3,5-diazanaphthalen-2-one (E-141): Example E-141 (0.022 g) was synthesized by following procedure as described for the synthesis of E-01 (step-6) using 2-fluoro-3-{[2-oxo-7-(3-pyridazinyloxy)-3,4-dihydro-2H-1-oxa-3,5-diazanaphth-3-yl]methyl}aniline (E-141i) (0.17 g, 0.463 mmol) as the starting material. The final compound was purified by reverse phase preparative HPLC purification [using X Select Phenyl Hexyl (19 mm×250 mm, 5 m particle size) column, 5% ammonia in water and acetonitrile as mobile phase with 3 mL/min flow rate]. LCMS (ES) m/z: 461.3 [M+H]⁺. ¹H NMR (400 MHz, DMSO-d₆): δ 9.47 (s, 1H); 9.06 (d, J=4.4 Hz, 1H); 8.33 (d, J=2.0 Hz, 1H); 7.85-7.81 (m, 1H); 7.67 (d, J=2.0 Hz, 1H); 7.60 (d, J=8.8 Hz, 1H); 7.40 (t, J=7.2 Hz, 1H); 7.28-7.21 (m, 2H); 7.18-7.14 (m, 1H); 4.70 (s, 2H); 4.60 (s, 2H); 2.52 (d, J=4.4 Hz, 3H).

### Example 142: Preparation of 3-({3-[(methylsulfamoyl)amino]phenyl}methyl)-2-oxo-3,4-dihydro-2H-1,3-benzoxazin-7-yl N,N-dimethylcarbamate (E-142; Compound 28)

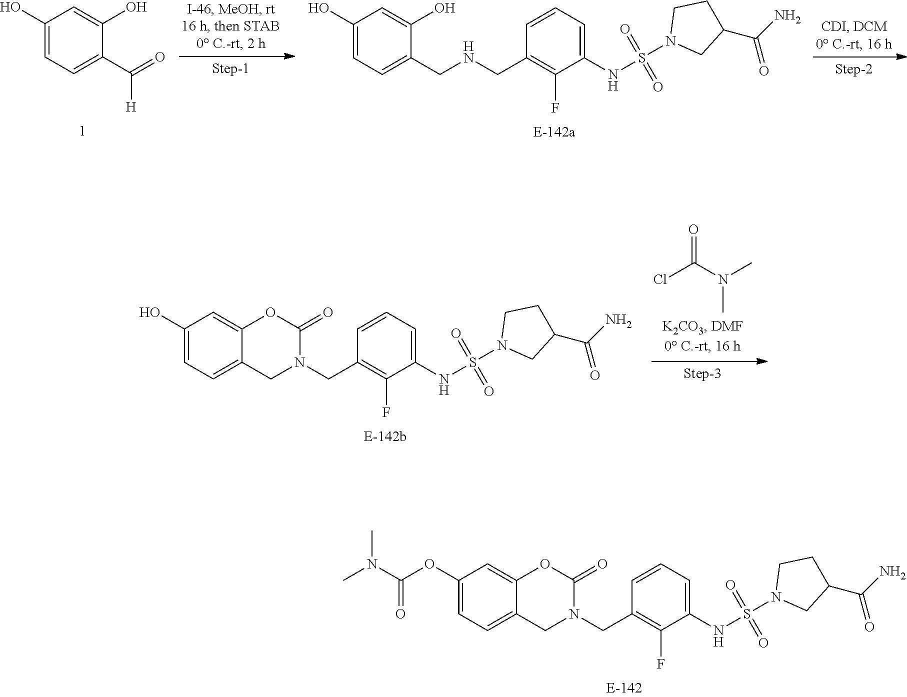

Step-1: Synthesis of 1-{[3-({[(2,4-dihydroxyphenyl)methyl]amino}methyl)-2-fluorophenyl]sulfamoyl}pyrrolidine-3-carboxamide (E-142a): Compound E-142a (0.4 g) was synthesized by following procedure as described for the synthesis of E-01 (step-1) using of 1-{[3-(aminomethyl)-2-fluorophenyl]sulfamoyl}pyrrolidine-3-carboxamide (I-46) (1.23 g, 3.89 mmol), 2,4-dihydroxybenzaldehyde (1) (0.322 g, 2.33 mmol) and sodium triacetoxyborohydride (1.24 g, 5.83 mmol) as the starting materials. LCMS (ES) m/z: 439.1 [M+H]⁺.

Step-2: Synthesis of 1-({2-fluoro-3-[(7-hydroxy-2-oxo-3,4-dihydro-2H-1,3-benzoxazin-3-yl)methyl]phenyl}sulfamoyl)pyrrolidine-3-carboxamide (E-142b): Compound E-142b (0.095 g) was synthesized by following procedure as described for the synthesis of E-01 (step-2) using 1-{[3-({[(2,4-dihydroxyphenyl)methyl]amino}methyl)-2-fluorophenyl]sulfamoyl}pyrrolidine-3-carboxamide (E-142a) (0.2 g, 0.456 mmol) as the starting material. LCMS (ES) m/z: 465.1 [M+H]⁺.

Step-3: Synthesis of 3-[(3-{[(3-carbamoylpyrrolidin-1-yl)sulfonyl]amino}-2-fluorophenyl)methyl]-2-oxo-3,4-dihydro-2H-1,3-benzoxazin-7-yl N,N-dimethylcarbamate (E-142): Example E-142 (0.028 g) was synthesized by following procedure as described for the synthesis of E-01 (step-4) using 1-({2-fluoro-3-[(7-hydroxy-2-oxo-3,4-dihydro-2H-1,3-benzoxazin-3-yl)methyl]phenyl}sulfamoyl)pyrrolidine-3-carboxamide (E-142b) (0.07 g, 0.151 mmol) as the starting material. The final compound was purified by reverse phase preparative HPLC purification [using Eclipse C18 (150 mm×21 mm, 7 μm particle size) column, 0.1% ammonia in water and acetonitrile as mobile phase with 19 mL/min flow rate]. LCMS (ES) m/z: 536.4 [M+H]⁺. ¹H NMR (400 MHz, DMSO-d₆): δ 9.69 (s, 1H); 7.44-7.38 (m, 2H); 7.23 (d, J=8.0 Hz, I H); 7.18-7.12 (m, 2H); 6.97 (br s, 1H); 6.91-6.86 (m, 2H); 4.67 (s, 2H); 4.47 (s, 2H); 3.41-3.14 (m, 4H); 3.03 (s, 3H); 2.92-2.84 (m, 4H); 2.00-1.85 (m, 2H).

### Example 143: Preparation of dimethylamino-3-{[2-fluoro-3-(2-oxo-1,3-oxazolidin-3-yl)phenyl]methyl}-2-oxo-3,4-dihydro-2H-1,3-benzoxazin-7-ylformylate (E-143; Compound 27)

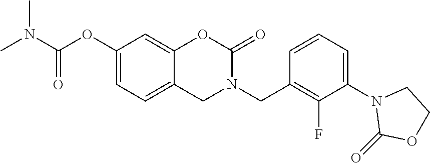 [SMILES: CN(C)C(=O)Oc1ccc2c(c1)OC(=O)N(Cc1cccc(N3CCOC3=O)c1F)C2]

E-143 was synthesized by following similar synthesis sequence and experimental procedures as described for the synthesis of E-142, using 2,4-dihyroxybenzaldehyde and I-47 as the starting materials. LCMS (ES) m/z: 430.25 [M+H]⁺. ¹H NMR (400 MHz, DMSO-d₆): δ 7.52-7.48 (m, 1H); 7.37-7.31 (m, 1H); 7.28-7.22 (m, 2H); 6.93-6.87 (m, 2H); 4.69 (s, 2H); 4.51 (s, 2H); 4.47 (t, J=8.0 Hz, 2H); 4.01 (t, J=8.0, 2H); 3.03 (s, 3H); 2.90 (s, 3H).

### Example 144: Preparation of 3-(2-fluoro-3-((2-oxooxazolidin-3-yl)methyl)benzyl)-2-oxo-3,4-dihydro-2H-benzo[e][1,3]oxazin-7-yl dimethylcarbamate (E-144; Compound 21)

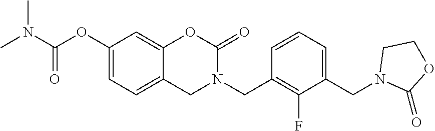 [SMILES: CN(C)C(=O)Oc1ccc2c(c1)OC(=O)N(Cc1cccc(CN3CCOC3=O)c1F)C2]

E-144 was synthesized by following similar synthesis sequence and experimental procedures as described for the synthesis of E-142, using 2,4-dihyroxybenzaldehyde and I-48 as the starting materials. LCMS (ES) m/z: 444.3 [M+H]⁺. ¹H NMR (400 MHz, DMSO-d₆): δ 7.40-7.29 (m, 2H); 7.25-7.19 (m, 2H); 6.92-6.88 (m, 2H); 4.68 (s, 2H); 4.49 (s, 2H); 4.42 (s, 2H); 4.26 (t, J=7.6 Hz, 2H); 3.45 (t, J=8.0 Hz, 2H); 3.02 (s, 3H); 2.90 (s, 3H).

### Example 145: Preparation of dimethylamino-3-({2-fluoro-3-[(2-oxo-1-imidazolidinyl)methyl]phenyl}methyl)-2-oxo-3,4-dihydro-2H-1,3-benzoxazin-7-ylformylate (E-145; Compound 4)

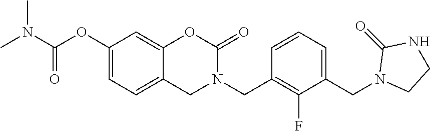 [SMILES: CN(C)C(=O)Oc1ccc2c(c1)OC(=O)N(Cc1cccc(CN3CCNC3=O)c1F)C2]

E-145 was synthesized by following similar synthesis sequence and experimental procedures as described for the synthesis of E-142, using 2,4-dihyroxybenzaldehyde and I-49 as the starting materials. LCMS (ES) m/z: 443.35 [M+H]⁺. ¹H NMR (400 MHz, DMSO-d₆): δ 7.36-7.16 (m, 4H); 6.92-6.86 (m, 2H); 6.46 (s, 1H); 4.67 (s, 2H); 4.48 (s, 2H); 4.30 (s, 2H); 3.23 (s, 4H); 3.02 (s, 3H); 2.90 (s, 3H).

### Example 146: Preparation of 3-(2-fluoro-3-((2-oxooxazolidin-3-yl)methyl)benzyl)-7-(pyridazin-3-yloxy)-3,4-dihydro-2H-benzo[e][1,3]oxazin-2-one (E-146; Compound 7)

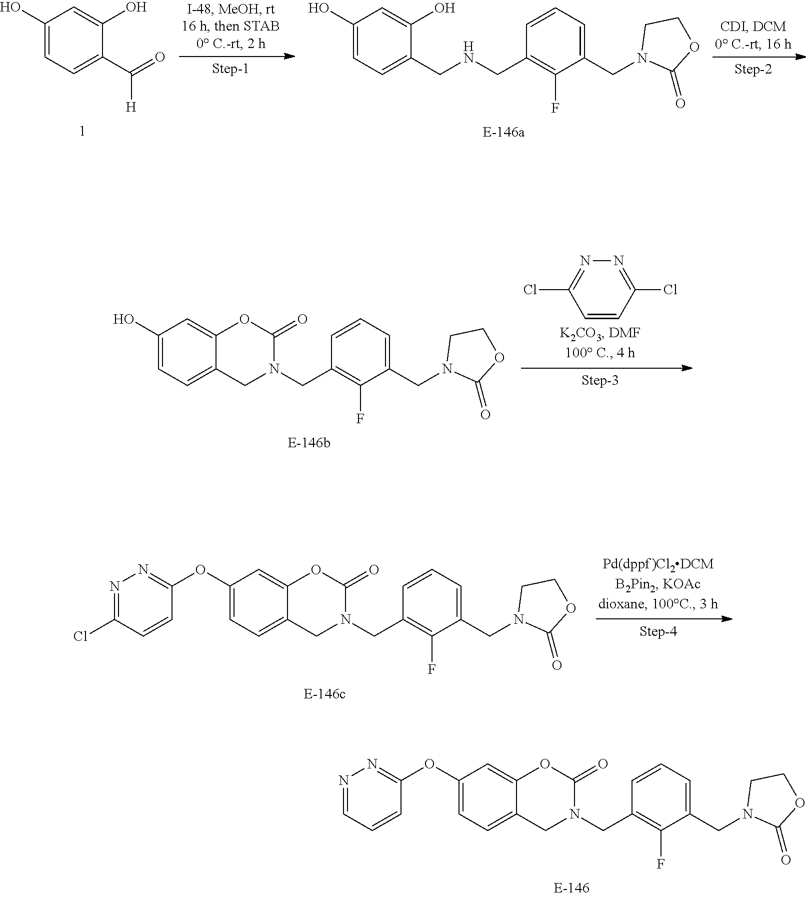 [SMILES: O=Cc1ccc(O)cc1O ; O=C1OCCN1Cc1cccc(CNCc2ccc(O)cc2O)c1F ; Clc1ccc(Cl)nn1 ; O=C1OCCN1Cc1cccc(CN2Cc3ccc(O)cc3OC2=O)c1F ; O=C1OCCN1Cc1cccc(CN2Cc3ccc(Oc4ccc(Cl)nn4)cc3OC2=O)c1F ; O=C1OCCN1Cc1cccc(CN2Cc3ccc(Oc4cccnn4)cc3OC2=O)c1F]

Step-1: Synthesis of 3-{[3-({[(2,4-dihydroxyphenyl)methyl]amino}methyl)-2-fluorophenyl]methyl}-1,3-oxazolidin-2-one (E-146a): Compound E-146a (1.4 g) was synthesized by following procedure as described for the synthesis of E-01 (step-1) using of ({2-fluoro-3-[(2-oxo-1,3-oxazolidin-3-yl)methyl]phenyl}methyl)amine (1-48) (4.0 g, 17.8 mmol) and 2,4-dihydroxybenzaldehyde (1) (1.72 g, 12.5 mmol) as the starting materials. LCMS (ES) m/z: 347.3 [M+H]⁺.

Step-2: Synthesis of 3-({2-fluoro-3-[(2-oxo-1,3-oxazolidin-3-yl)methyl]phenyl}methyl)-7-hydroxy-3,4-dihydro-2H-1,3-benzoxazin-2-one (E-146b): Compound E-146b (0.65 g) was synthesized by following procedure as described for the synthesis of E-01 (step-2) using 3-{[3-({1[(2,4-dihydroxyphenyl)methyl]amino}methyl)-2-fluorophenyl]methyl}-1,3-oxazolidin-2-one (E-146a) (1.80 g) as the starting material. LCMS (ES) m/z: 371.2 [M−H]⁺.

Step-3: Synthesis of 7-(6-chloro-3-pyridazinyloxy)-3-({2-fluoro-3-[(2-oxo-1,3-oxazolidin-3-yl)methyl]phenyl}methyl)-3,4-dihydro-2H-1,3-benzoxazin-2-one (E-146c): Compound E-146c (0.50 g) was synthesized by following procedure as described for the synthesis of E-34 (step-1) using 3-({2-fluoro-3-[(2-oxo-1,3-oxazolidin-3-yl)methyl]phenyl}methyl)-7-hydroxy-3,4-dihydro-2H-1,3-benzoxazin-2-one (E-146b) (0.65 g, 1.34 mmol) and 3,6-dichloropyridazine (0.26 g, 1.75 mmol) as the starting material. LCMS (ES) m/z: 485.2 [M+H]⁺.

Step-4: Synthesis of 3-(2-fluoro-3-((2-oxooxazolidin-3-yl)methyl)benzyl)-7-(pyridazin-3-yloxy)-3,4-dihydro-2H-benzo[e][1,3]oxazin-2-one (E-146): Example E-146 (0.078 g) was synthesized by following procedure as described for the synthesis of E-34 (step-2) using 37-(6-chloro-3-pyridazinyloxy)-3-({2-fluoro-3-[(2-oxo-1,3-oxazolidin-3-yl)methyl]phenyl}methyl)-3,4-dihydro-2H-1,3-benzoxazin-2-one (E-146c) (0.5 g, 1.03 mmol) as the starting material. The final compound was purified by reverse phase preparative HPLC purification [using Eclipse C18 (150 mm×21 mm, 7 μm particle size) column, 0.1% ammonia in water and acetonitrile as mobile phase with 19 mL/min flow rate]. LCMS (ES) m/z: 451.3 [M+H]⁺. ¹H NMR (400 MHz, DMSO-d₆): δ 9.02 (dd, J₁=1.2 Hz, J₂=4.4 Hz, 1H); 7.81-7.76 (m, 1H); 7.49 (dd, J₁=1.2 Hz, J₂=8.8 Hz, 1H); 7.42-7.30 (m, 3H); 7.23 (t, J=7.6, 1H); 7.04-6.99 (m, 2H); 4.70 (s, 2H); 4.53 (s, 2H); 4.43 (s, 2H); 4.27 (t, J=8.0, 2H); 3.46 (t, J=8.0, 2H).

### Example 147: Preparation of 3-{[2-fluoro-3-(2-oxo-1,3-oxazolidin-3-yl)phenyl]methyl}-7-(3-pyridazinyloxy)-3,4-dihydro-2H-1,3-benzoxazin-2-one (E-147; Compound 26)

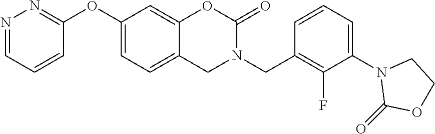 [SMILES: O=C1Oc2cc(Oc3cccnn3)ccc2CN1Cc1cccc(N2CCOC2=O)c1F]

E-147 was synthesized by following similar synthesis sequence and experimental procedures as described for the synthesis of E-146, using 2,4-dihydroxybenzaldehyde and I-47 as the starting materials. LCMS (ES) m/z: 437.25 [M+H]⁺. ¹H NMR (400 MHz, DMSO-d₆): δ 9.03 (d, J=4.4 Hz, 1H); 7.80-7.76 (m, 1H); 7.53-7.48 (m, 2H); 7.40-7.32 (m, 2H); 7.28 (t, J=7.6 Hz, 1H); 7.03-6.99 (m, 2H); 4.72 (s, 2H); 4.55 (s, 2H); 4.48 (t, J=7.6 Hz, 2H); 4.03 (t, J=8.0 Hz, 2H).

### Example 148: Preparation of 3-(2-fluoro-3-((3-oxomorpholino)methyl)benzyl)-7-(pyridazin-3-yloxy)-3,4-dihydro-2H-benzo[e][1,3]oxazin-2-one (E-148; Compound 3)

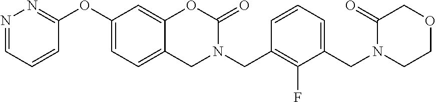 [SMILES: O=C1COCCN1Cc1cccc(CN2Cc3ccc(Oc4cccnn4)cc3OC2=O)c1F]

E-148 was synthesized by following synthesis sequence and procedures as described for the synthesis of E-146, using 2,4-dihydroxybenzaldehyde and I-50 as the starting materials. LCMS (ES) m/z: 465.3 [M+H]⁺. ¹H NMR (400 MHz, DMSO-d₆): h 9.02 (dd, J₁=1.2 Hz, J₂=4.4 Hz, 1H); 7.80-7.76 (m, 1H); 7.48 (dd, J₁=1.2 Hz, J₂=8.8 Hz, 1H); 7.40-7.18 (m, 4H); 7.02 (s, 1H); 7.04-6.98 (m, 2H); 4.70 (s, 2H); 4.62 (s, 2H); 4.53 (s, 2H); 4.11 (s, 2H); 3.84 (t, J=5.2 Hz, 2H); 3.30 (t, J=5.2 Hz, 2H).

### Example 149: Preparation of 3-({2-fluoro-3-[(2-oxo-1-imidazolidinyl)methyl]phenyl}methyl)-7-(3-pyridazinyloxy)-3,4-dihydro-2H-1,3-benzoxazin-2-one (E-149; Compound 6)

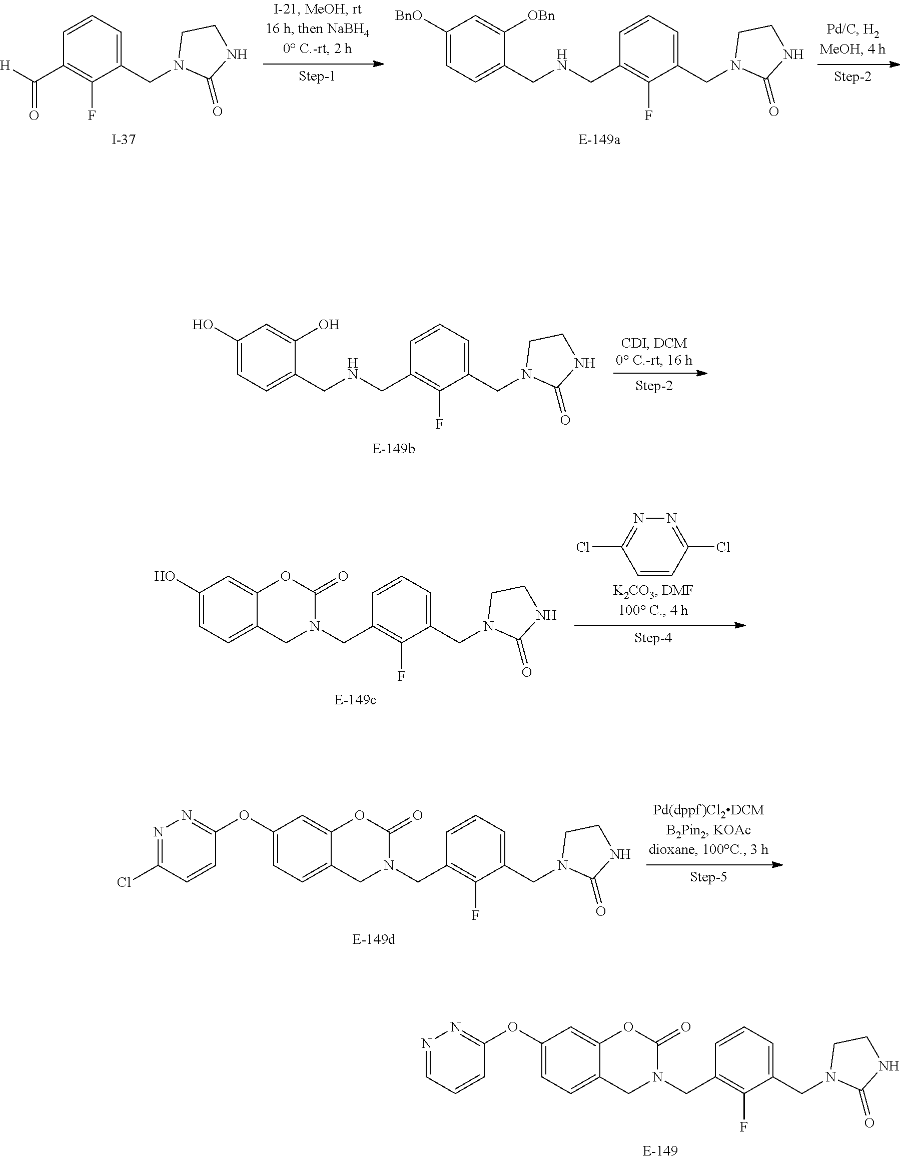

Step-1: Synthesis of {[2,4-bis(benzyloxy)phenyl]methyl}({2-fluoro-3-[(2-oxo-1-imidazolidinyl)methyl]phenyl}methyl)amine (E-149a): Compound E-149a (1.5 g) was synthesized by following procedure as described for the synthesis of E-01 (step-1) using {[2,4-bis(benzyloxy)phenyl]methyl}amine (I-21) (1.5 g, 4.7 mmol) and 2-fluoro-3-[(2-oxo-1-imidazolidinyl)methyl]benzaldehyde (I-37) (835 mg, 3.76 mmol) as the starting materials. LCMS (ES) m/z: 526.1 [M+H]⁺.

Step-2: Synthesis of 1-{[3-({[(2,4-dihydroxyphenyl)methyl]amino}methyl)-2-fluorophenyl]methyl}-2-imidazolidinone (E-149b): Compound E-149b (0.55 g) was synthesized by following procedure as described for the synthesis of E-97 (step-2) using 1 {[2,4-bis(benzyloxy)phenyl]methyl}({2-fluoro-3-[(2-oxo-1-imidazolidinyl)methyl]phenyl}methyl)amine (E-149a) (1.5 g, 2.85 mmol) as the starting material. LCMS (ES) m/z: 346.3 [M+H]⁺.

Step-3: Synthesis of 3-({2-fluoro-3-[(2-oxo-1-imidazolidinyl)methyl]phenyl}methyl)-7-hydroxy-3,4-dihydro-2H-1,3-benzoxazin-2-one (E-149c): Compound E-149c (0.32 g) was synthesized by following procedure as described for the synthesis of E-01 (step-2) using 11-{[3-({[(2,4-dihydroxyphenyl)methyl]amino}methyl)-2-fluorophenyl]methyl}-2-imidazolidinone (E-149b) (550 mg, 1.59 mmol) as the starting material. LCMS (ES) m/z: 372.3 [M+H]⁺.

Step-4: Synthesis of 7-(6-chloro-3-pyridazinyloxy)-3-({2-fluoro-3-[(2-oxo-1-imidazolidinyl)methyl]phenyl}methyl)-3,4-dihydro-2H-1,3-benzoxazin-2-one (E-149d): Compound E-149d (0.25 g) was synthesized by following procedure as described for the synthesis of E-34 (step-1) using 3-({2-fluoro-3-[(2-oxo-1-imidazolidinyl)methyl]phenyl}methyl)-7-hydroxy-3,4-dihydro-2H-1,3-benzoxazin-2-one (E-149c) (320 mg, 862 Pmol) as the starting material. LCMS (ES) m/z: 483.9 [M+H]⁺.

Step-5: Synthesis of 3-({2-fluoro-3-[(2-oxo-1-imidazolidinyl)methyl]phenyl}methyl)-7-(3-pyridazinyloxy)-3,4-dihydro-2H-1,3-benzoxazin-2-one (E-149): Example E-149 (0.1 g) was synthesized by following procedure as described for the synthesis of E-34 (step-2) using 7-(6-chloro-3-pyridazinyloxy)-3-({2-fluoro-3-[(2-oxo-1-imidazolidinyl)methyl]phenyl}methyl)-3,4-dihydro-2H-1,3-benzoxazin-2-one (E-149d) (250 mg, 517 μmol) as the starting material. The final compound was purified by reverse phase preparative HPLC purification [using Eclipse C18 (150 mm×21 mm, 7 μm particle size) column, 0.1% ammonia in water and acetonitrile as mobile phase with 19 mL/min flow rate]. LCMS (ES) m/z: 450.3 [M+H]⁺. ¹H NMR (400 MHz, DMSO-d₆): δ 9.03 (dd, J₁=1.2 Hz, J₂=4.4 Hz, 1H); 7.80-7.76 (m, 1H); 7.49 (dd, J₁=1.2 Hz, J₂=8.8 Hz, 1H); 7.39-7.26 (m, 3H); 7.21 (t, J=7.6 Hz, 1H); 7.03-6.97 (m, 2H); 6.43 (s, 1H); 4.70 (s, 2H); 4.53 (s, 2H); 4.32 (s, 2H); 3.25 (s, 4H).

### Example 150: Preparation of 2-[3-({2-fluoro-3-[(methylsulfamoyl)amino]phenyl}methyl)-2-oxo-3,4-dihydro-2H-1,3-benzoxazin-7-yl]-N,N-dimethylacetamide (E-150; Compound 134)

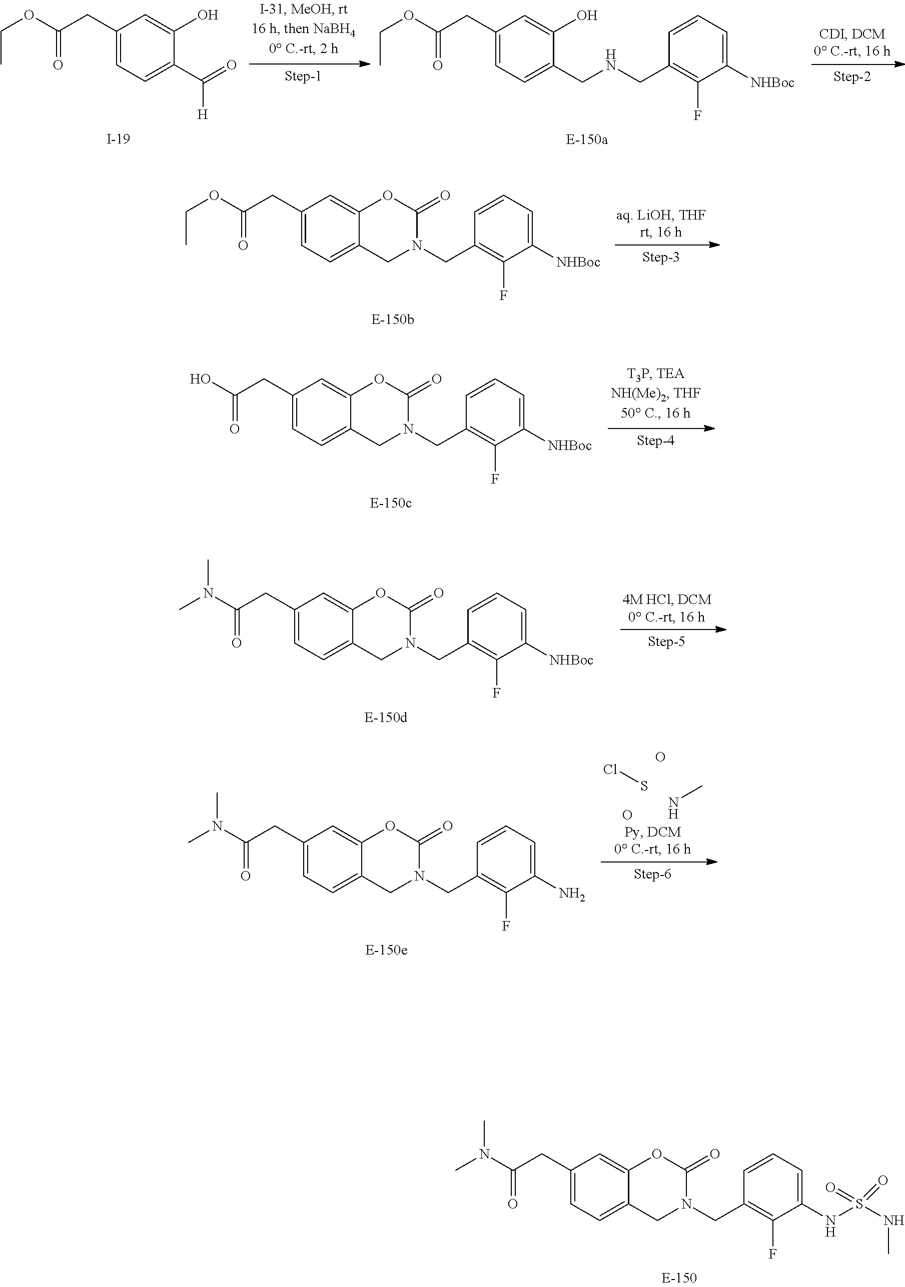

Step-1: Synthesis of ethyl 2-[4-({[(3-{[(tert-butoxy)carbonyl]amino}-2-fluorophenyl)methyl]amino}methyl)-3-hydroxyphenyl]acetate (E-150a): Compound E-150a (2.2 g) was synthesized by following procedure as described for the synthesis of E-01 (step-1) using ethyl 2-(4-formyl-3-hydroxyphenyl)acetate (I-19) (1.8 g, 8.64 mmol) and tert-butyl N-|3-(aminomethyl)-2-fluorophenylicarbamate (I-31) (2.08 g, 8.64 mmol) as the starting materials. LCMS (ES) m/z: 433.39 [M+H]⁺.

Step-2: Synthesis of ethyl 2-{3-[(3-1[(tert-butoxy)carbonyl]amino}-2-fluorophenyl)methyl]-2-oxo-3,4-dihydro-2H-1,3-benzoxazin-7-yl}acetate (E-150b): Compound E-150b (1.6 g) was synthesized by following procedure as described for the synthesis of E-01 (step-2) using ethyl 2-[4-({[(3-{[(tert-butoxy)carbonyl]amino}-2-fluorophenyl)methylIamino}methyl)-3-hydroxyphenylIacetate (E-150a) (2.2 g, 5.09 mmol) as the starting material. LCMS (ES) m/z: 457.43 [M−H]⁺.

Step-3: Synthesis of 2-{3-[(3-{[(tert-butoxy)carbonyl]amino}-2-fluorophenyl)methyl]-2-oxo-3,4-dihydro-2H-1,3-benzoxazin-7-yl}acetic acid (E-150c): To a stirred solution of ethyl 2-{3-[(3-{[(tert-butoxy)carbonyl]amino}-2-fluorophenyl)methyl]-2-oxo-3,4-dihydro-2H-1,3-benzoxazin-7-yl}acetate (E-150b) (1.6 g, 3.49 mmol) in THE (16 mL) was added an aqueous solution of LiOH (0.29 g, 6.98 mmol, in 3 mL water) and the reaction mixture was stirred at room temperature. The progress of the reaction was monitored by TLC/LCMS analysis. After completion of the reaction (16 h), the reaction mixture was acidified with 2N citric acid (10 mL) and extraction was carried out using EtOAc (30 mL×3). The combined organic extracts were washed with water (50 mL), brine (50 mL), dried over anhydrous Na₂SO₄, filtered and concentrated under reduced pressure to afford desired compound E-150c (0.65 g) as a yellow solid. ¹H NMR (400 MHz, DMSO-d₆): δ 12.25 (br s, 1H); 8.98 (s, 1H); 7.55-7.53 (m, 1H); 7.15 (d, J=7.6 Hz, 1H); 7.13-7.07 (m, 2H); 7.01 (d, J=8.0 Hz, 1H); 6.96 (s, 1H); 4.66 (s, 2H); 4.45 (s, 2H); 3.58 (s, 2H); 1.44 (s, 9H).

Step-4: Synthesis of tert-butyl N-[3-({7-[(dimethylcarbamoyl)methyl]-2-oxo-3,4-dihydro-2H-1,3-benzoxazin-3-yl}methyl)-2-fluorophenyl]carbamate (E-150d): To a stirred solution of 2-{3-[(3-1[(tert-butoxy)carbonyl]amino}-2-fluorophenyl)methyl]-2-oxo-3,4-dihydro-2H-1,3-benzoxazin-7-yl}acetic acid (E-150c) (0.35 g, 813 μmol) in THE (4.04 mL, 49.6 mmol) was added TEA (0.340 mL, 2.44 mmol), dimethylamine (0.267 g, 3.25 mmol) and T₃P (50% in EtOAc) (1.5 mL, 2.44 mmol) at 0° C. The reaction mixture was then stirred at 50° C., while monitoring reaction progress by TLC/LCMS analysis. After completion the reaction (16 h), the reaction mixture was cooled to room temperature and then quenched with addition of water (15 mL). Extraction was carried out using EtOAc (20 mL×3); the combined organic extracts were washed with water (20 mL), brine (30 mL), dried over anhydrous Na₂SO₄, filtered and concentrated under reduced pressure. The residue was purified by Combi-Flash (using gradient elution of 0-50% of EtOAc in heptane) to afford desired compound E-150d (0.25 g) as a white solid. LCMS (ES) m/z: 456.72 [M−H]⁺.

Step-5: Synthesis of 2-{3-[(3-amino-2-fluorophenyl)methyl]-2-oxo-3,4-dihydro-2H-1,3-benzoxazin-7-yl}-N,N-dimethylacetamide (E-150e): Compound E-150e (0.180 g) was synthesized by following procedure as described for the synthesis of E-17 (step-3) using tert-butyl N-[3-({7-[(dimethylcarbamoyl)methyl]-2-oxo-3,4-dihydro-2H-1,3-benzoxazin-3-yl}methyl)-2-fluorophenyl]carbamate (E-150d) (0.25 g, 0.546 mmol) as the starting material. LCMS (ES) m/z: 358.27 [M+H]⁺.

Step-6: Synthesis of 2-{3-[(3-amino-2-fluorophenyl)methyl]-2-oxo-3,4-dihydro-2H-1,3-benzoxazin-7-yl}-N,N-dimethylacetamide (E-150): Example E-150 (0.008 g) was synthesized by following procedure as described for the synthesis of E-01 (step-6) using 2-{3-[(3-amino-2-fluorophenyl)methyl]-2-oxo-3,4-dihydro-2H-1,3-benzoxazin-7-yl}-N,N-dimethylacetamide (E-150e) (0.17 g, 0.476 mmol) as the starting material. The final compound was purified by reverse phase preparative HPLC purification [using XSELECT CSH C18 (19 mm×250 mm, 5 μm particle size) column, 5% ammonium bicarbonate in water and acetonitrile as mobile phase]. LCMS (ES) m/z: 451.19 [M+H]⁺. ¹H NMR (400 MHz, DMSO-d₆): δ 9.42 (s, 1H); 7.40-7.36 (m, 1H); 7.22 (q, J=4.8 Hz, 1H); 7.17-7.11 (m, 3H); 6.97 (dd, J₁=1.6 Hz, J₂=8.0 Hz, 1H); 6.89 (d, J=1.2 Hz, 1H); 4.66 (s, 2H); 4.46 (s, 2H); 3.68 (s, 2H); 3.00 (s, 3H); 2.82 (s, 3H); 2.51 (d, J=4.4 Hz, 3H).

### Example 151: Preparation of 2,2-difluoro-2-[3-({2-fluoro-3-(methylsulfamoyl)amino]phenyl}methyl)-2-oxo-3,4-dihydro-2H-1,3-benzoxazin-7-yl]-N,N-dimethylacetamide (E-151; Compound 133)

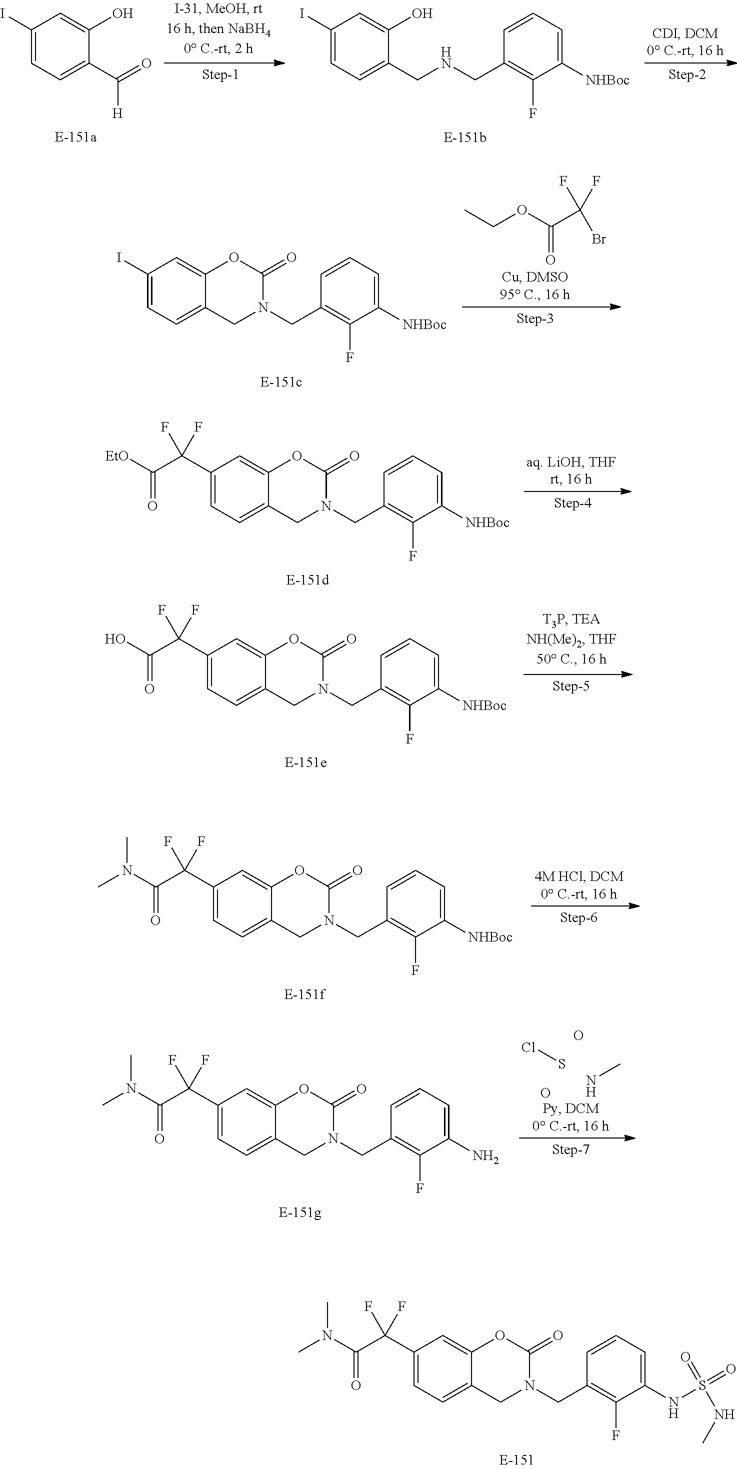

Step-1: Synthesis of tert-butyl N-[2-fluoro-3-({[(2-hydroxy-4-iodophenyl)methyl]amino}methyl)phenyl]carbamate (E-151b): Compound E-151b (4.1 g) was synthesized by following procedure as described for the synthesis of E-01 (step-1) using 2-hydroxy-4-iodobenzaldehyde (E-151a) (4 g, 16.1 mmol) and tert-butyl N-[3-(aminomethyl)-2-fluorophenyl]carbamate (I-31) (3.88 g, 16.1 mmol) as the starting materials. LCMS (ES) m/z: 473.2 [M+H]⁺.

Step-2: Synthesis of tert-butyl N-{2-fluoro-3-[(7-iodo-2-oxo-3,4-dihydro-2H-1,3-benzoxazin-3-yl)methyl]phenyl}carbamate (E-151c): Compound E-151c (1.6 g) was synthesized by following procedure as described for the synthesis of E-01 (step-2) using tert-butyl N-[2-fluoro-3-({[(2-hydroxy-4-iodophenyl)methyl]amino}methyl)phenyl]carbamate (E-151b) (4.1 g, 8.68 mmol) as the starting material. LCMS (ES) m/z: 497.3 [M−H]⁺.

Step-3: Synthesis of ethyl 2-{3-[(3-{[(tert-butoxy)carbonyl]amino}-2-fluorophenyl)methyl]-2-oxo-3,4-dihydro-2H-1,3-benzoxazin-7-yl}-2,2-difluoroacetate (E-151d): To a stirred solution of ethyl 2-bromo-2,2-difluoroacetate (1.55 mL, 12 mmol) in DMSO (20 mL) was added copper (0.765 g, 12 mmol) and tert-butyl N-{2-fluoro-3-[(7-iodo-2-oxo-3,4-dihydro-2H-1,3-benzoxazin-3-yl)methyl]phenyl}carbamate (E-151c) (3 g, 6.02 mmol) at room temperature. The reaction mixture was then stirred at 95° C. for 16 h. The reaction mixture was then cooled to room temperature and water (40 mL) was added to it. Extraction was carried out using EtOAc (30 mL×3); the combined organic extracts were washed with water (50 mL×3), brine (50 mL), dried over anhydrous Na₂SO₄, filtered and concentrated under reduced pressure. The residue was purified by Combi-Flash (using gradient elution of 0-10% of EtOAc in heptane) to afford desired compound E-151d (2 g) as a white solid. LCMS (ES) m/z: 493.43 [M−H]⁺.

Step-4: Synthesis of 2-{3-[(3-{[(tert-butoxy)carbonyl]amino}-2-fluorophenyl)methyl]-2-oxo-3,4-dihydro-2H-1,3-benzoxazin-7-yl}-2,2-difluoroacetic acid (E-151e): Compound E-151e (1.2 g) was synthesized by following procedure as described for the synthesis of E-150 (step-3) using ethyl 2-{3-[(3-{[(tert-butoxy)carbonyl]amino}-2-fluorophenyl)methyl]-2-oxo-3,4-dihydro-2H-1,3-benzoxazin-7-yl}-2,2-difluoroacetate (E-151d) (1.5 g, 3.03 mmol) as the starting material. LCMS (ES) m/z: 465.3 [M−H]⁺.

Step-5: Synthesis of tert-butyl N-[3-({7-[(dimethylcarbamoyl)difluoromethyl]-2-oxo-3,4-dihydro-2H-1,3-benzoxazin-3-yl}methyl)-2-fluorophenyl]carbamate (E-151f): Compound E-151f (0.570 g) was synthesized by following procedure as described for the synthesis of E-150 (step-4) using 2-{3-[(3-{[(tert-butoxy)carbonyl]amino}-2-fluorophenyl)methyl]-2-oxo-3,4-dihydro-2H-1,3-benzoxazin-7-yl}-2,2-difluoroacetic acid (E-151e) (1.2 g, 2.57 mmol) as the starting material. LCMS (ES) m/z: 492.29 [M−H]⁺.

Step-6: Synthesis of 2-{3-[(3-amino-2-fluorophenyl)methyl]-2-oxo-3,4-dihydro-2H-1,3-benzoxazin-7-yl}-2,2-difluoro-N,N-dimethylacetamide (E-151g): Compound E-151g (0.450 g) was synthesized by following procedure as described for the synthesis of E-17 (step-3) using tert-butyl N-[3-({7-[(dimethylcarbamoyl)difluoromethyl]-2-oxo-3,4-dihydro-2H-1,3-benzoxazin-3-yl}methyl)-2-fluorophenyl]carbamate (E-151f) (0.57 g, 1.16 mmol) as the starting material. LCMS (ES) m/z: 394.31 [M+H]⁺.

Step-7: Synthesis of 2,2-difluoro-2-[3-({2-fluoro-3-(methylsulfamoyl)amino]phenyl}methyl)-2-oxo-3,4-dihydro-2H-1,3-benzoxazin-7-yl]-N,N-dimethylacetamide (E-151): Example E-151 (0.075 g) was synthesized by following procedure as described for the synthesis of E-01 (step-6) using 2-{3-[(3-amino-2-fluorophenyl)methyl]-2-oxo-3,4-dihydro-2H-1,3-benzoxazin-7-yl}-2,2-difluoro-N,N-dimethylacetamide (E-151g) (0.45 g, 1.14 mmol) as the starting material. The final compound was purified by reverse phase preparative HPLC purification [using XSELECT CSH C18 (19 mm×250 mm, 5 μm particle size) column, 5% ammonium bicarbonate and acetonitrile as mobile phase]. LCMS (ES) m/z: 485.40 [M−H]⁺. ¹H NMR (400 MHz, DMSO-d₆): δ 7.43-7.36 (m, 2H); 7.27 (d, J=8.0 Hz, 1H); 7.20 (s, 1H); 7.14-7.09 (m, 2H); 6.86 (br s, 2H); 4.66 (s, 2H); 4.55 (s, 2H); 2.94 (s, 3H); 2.89 (s, 3H); 2.51 (d, J=4.4 Hz, 3H).

### Example 152: Preparation of 2,2-difluoro-2-[3-({2-fluoro-3-[(methylsulfamoyl)amino]phenyl}methyl)-2-oxo-3,4-dihydro-2H-1,3-benzoxazin-7-yl]-N-methylacetamide. (E-152; Compound 101)

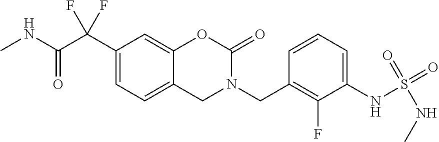 [SMILES: CNC(=O)C(F)(F)c1ccc2c(c1)OC(=O)N(Cc1cccc(NS(=O)(=O)NC)c1F)C2]

E-152 was synthesized by following similar synthesis sequence and experimental procedures as described for the synthesis of E-151, using key intermediates E-151a and I-31. (note: MeNH₂ was in step-5). LCMS (ES) m/z: 473.1 [M+H]⁺. ¹H NMR (400 MHz, DMSO-d₆): δ 9.43 (s, 1H); 8.93 (q, J=4.4 Hz, 1H); 7.41-7.36 (m, 2H); 7.34-7.30 (m, 1H); 7.25-7.20 (m, 2H); 7.16-7.11 (m, 2H); 4.67 (s, 2H); 4.54 (s, 2H); 2.67 (d, J=4.8 Hz, 3H); 2.51 (d, J=4.4 Hz, 3H).

### Example 153: Preparation of 2-(6-chloro-3-(2-fluoro-3-((N-methylsulfamoyl)amino)benzyl)-2-oxo-3,4-dihydro-2H-benzo[e][1,3]oxazin-7-yl)-2,2-difluoro-N,N-dimethylacetamide (E-153; Compound 94)

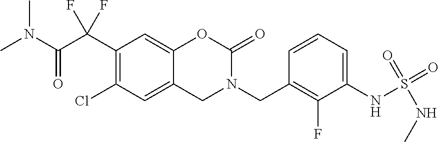 [SMILES: CNS(=O)(=O)Nc1cccc(CN2Cc3cc(Cl)c(C(F)(F)C(=O)N(C)C)cc3OC2=O)c1F]

E-153 was synthesized by following similar synthesis sequence and experimental procedures as described for the synthesis of E-151, using key intermediates I-20 and I-31. LCMS (ES) m/z: 519.03 [M−H]⁺. ¹H NMR (400 MHz, DMSO-d₆): δ 9.43 (s, 1H); 7.56 (s, 1H); 7.41-7.36 (m, 2H); 7.25-7.12 (m, 3H); 4.67 (s, 2H); 4.54 (s, 2H); 3.04 (s, 3H); 2.95 (s, 3H); 2.50 (d, J=4.4 Hz, 3H).

### Example 154: Preparation of 3-[1-{3-[(methylsulfamoyl)amino]phenyl}ethyl]-2-oxo-3,4-dihydro-2H-1,3-benzoxazin-7-yl N,N-dimethylcarbamate (E-154; Compound 145)

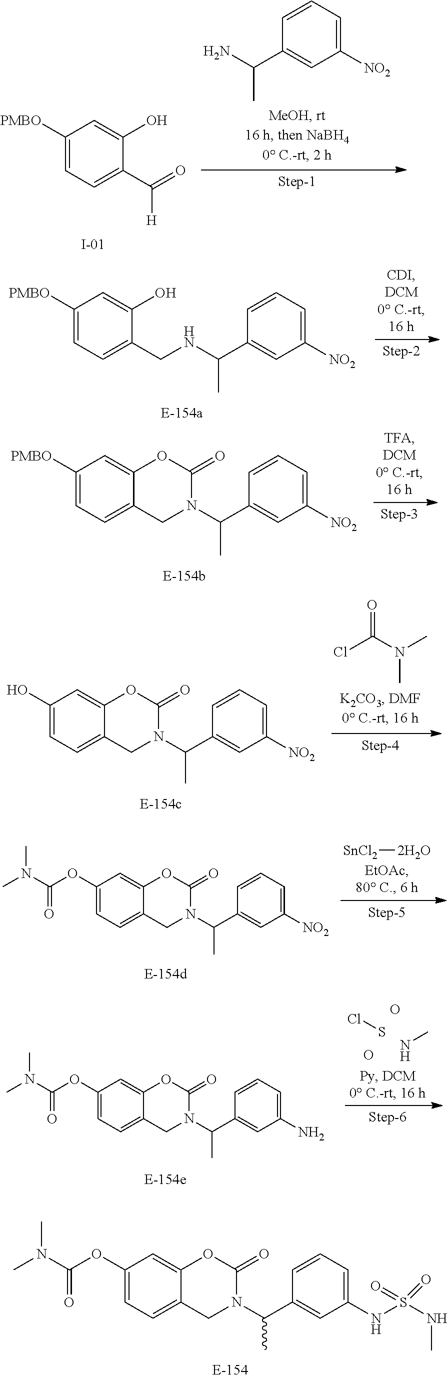

Step-1: Synthesis of 5-((4-methoxybenzyl)oxy)-2-(((1-(3-nitrophenyl)ethyl)amino)methyl)phenol (E-154a): Compound E-154a (1.0 g) was synthesized by following procedure as described for the synthesis of E-01 (step-1) using 2-hydroxy-4-((4-methoxybenzyl)oxy)benzaldehyde (I-01) (0.8 g, 3.1 mmol) and 1-(3-nitrophenyl)ethan-1-amine (0.618 g, 3.72 mmol) as the starting materials. LCMS (ES) m/z: 409.2 [M+H]⁺.

Step-2: Synthesis of 7-((4-methoxybenzyl)oxy)-3-(1-(3-nitrophenyl)ethyl)-3,4-dihydro-2H-benzo[e][1,3]oxazin-2-one (E-154b): Compound E-154b (0.8 g) was synthesized by following procedure as described for the synthesis of E-01 (step-2) using 5-((4-methoxybenzyl)oxy)-2-(((1-(3-nitrophenyl)ethyl)amino)methyl)phenol (E-154a) (1.0 g, 2.45 mmol) as the starting material. LCMS (ES) m/z: 435.2 [M+H]⁺.

Step-3: Synthesis of 7-hydroxy-3-(1-(3-nitrophenyl)ethyl)-3,4-dihydro-2H-benzo[e][1,3]oxazin-2-one (E-154c): Compound E-154c (0.450 g) was synthesized by following procedure as described for the synthesis of E-59 (step-3) using 7-((4-methoxybenzyl)oxy)-3-(1-(3-nitrophenyl)ethyl)-3,4-dihydro-2H-benzo[e][1,3]oxazin-2-one (E-154b) (0.8 g, 1.84 mmol) as the starting material. LCMS (ES) m/z: 315.2 [M+H]⁺.

Step-4: Synthesis of 3-(1-(3-nitrophenyl)ethyl)-2-oxo-3,4-dihydro-2H-benzo[e][1,3]oxazin-7-yl dimethylcarbamate (E-154d): Compound E-154d (0.40 g) was synthesized by following procedure as described for the synthesis of E-01 (step-4) using 7-hydroxy-3-(1-(3-nitrophenyl)ethyl)-3,4-dihydro-2H-benzo[e][1,3]oxazin-2-one (E-154c) (0.4 g, 1.27 μmol) as the starting material. LCMS (ES) m/z: 386.1 [M+H]⁺.

Step-5: Synthesis of 3-(1-(3-aminophenyl)ethyl)-2-oxo-3,4-dihydro-2H-benzo[e][1,3]oxazin-7-yl dimethylcarbamate (E-154e): Compound E-154e (0.2 g) was synthesized by following procedure as described for the synthesis of E-41 (step-3) using 3-(1-(3-nitrophenyl)ethyl)-2-oxo-3,4-dihydro-2H-benzo[e][1,3]oxazin-7-yl dimethylcarbamate (E-154d) (0.280 g, 0.0726 mmol) as the starting material. LCMS (ES) m/z: 356.1 [M+H]⁺.

Step-6: Synthesis of 3-(1-(3-((N-methylsulfamoyl)amino)phenyl)ethyl)-2-oxo-3,4-dihydro-2H-benzo[e][1,3]oxazin-7-yl dimethylcarbamate (E-154): Example E-154 (0.156 g) was synthesized by following procedure as described for the synthesis of E-01 (step-6) using 3-(1-(3-aminophenyl)ethyl)-2-oxo-3,4-dihydro-2H-benzo[e][1,3]oxazin-7-yl dimethylcarbamate (E-154e) (0.25 g, 703 mol) as the starting material. LCMS (ES) m/z: 449.2 [M+H]⁺. ¹H NMR (400 MHz, DMSO d₆): δ 9.67 (s, 1H); 7.27-7.10 (m, 4H); 7.08-6.86 (m, 4H); 5.52 (q, J=7.2 Hz, 1H); 4.46 (d, J=14.8 Hz, 1H); 3.95 (d, J=14.8 Hz, 1H); 3.02 (s, 3H); 2.90 (s, 3H); 2.40 (d, J=4.4 Hz, 3H); 1.56 (d, J=6.8 Hz, 3H).

### Examples 155, 156, and 157: Preparations of 3-(1-(2-fluoro-3-((N-methylsulfamoyl)amino)phenyl)ethyl)-2-oxo-3,4-dihydro-2H-benzo[e][1,3]oxazin-7-yl dimethylcarbamate (E-155; Compound 144), (S)-3-(1-(2-fluoro-3-((N-methylsulfamoyl)amino)phenyl)ethyl)-2-oxo-3,4-dihydro-2H-benzo[e][1,3]oxazin-7-yl dimethylcarbamate, and (R)-3-(1-(2-fluoro-3-((N-methylsulfamoyl)amino)phenyl)ethyl)-2-oxo-3,4-dihydro-2H-benzo[e][1,3]oxazin-7-yl dimethylcarbamate (E-156 and E-157; Compounds 143 and 142, respectively)

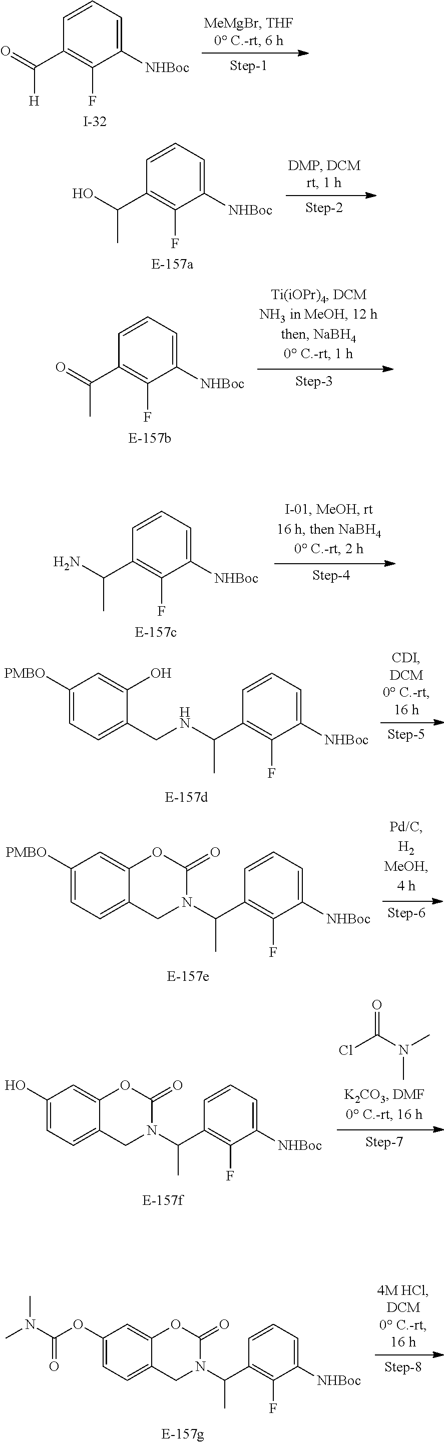

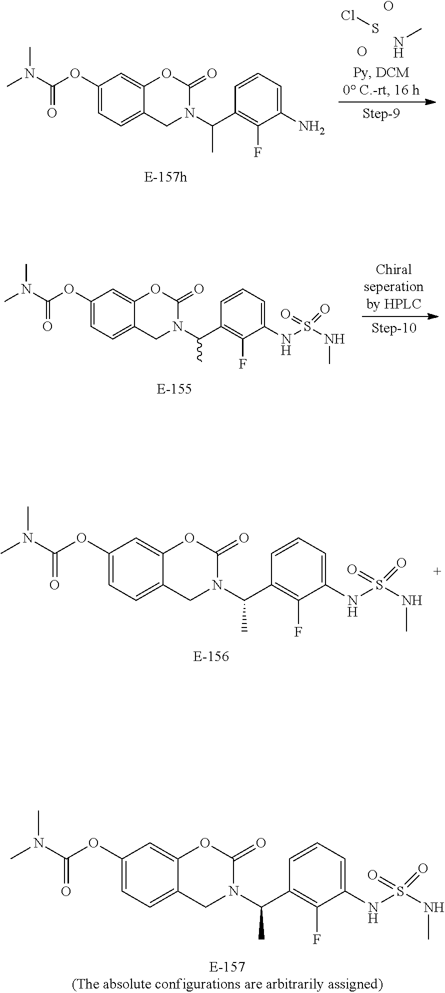

Step-1: Synthesis of tert-butyl N-[2-fluoro-3-(1-hydroxyethyl)phenyllcarbamate (E157a): To a stirred solution of tert-butyl N-(2-fluoro-3-formylphenyl)carbamate (I-32) (1.8 g, 7.52 mmol) in THF (20 mL) was added a 2 M solution of MeMgBr in THE (0.8 mL, 18.8 mmol) at 0° C. The reaction mixture was stirred at room temperature and the reaction progress was monitored by TLC/LCMS analysis. After completion of the reaction (6 h), the reaction mixture was quenched with addition of ice water (20 mL) and extraction was carried out using EtOAc (30 mL×3). The combined organic extracts were washed with water (50 mL), brine (50 mL), dried over anhydrous Na₂SO₄, filtered and concentrated under reduced pressure. The residue was purified by Combi-Flash (using gradient elution of 0-30% of EtOAc in heptane) to afford desired compound E-157a (1.2 g) as an off-white solid. LCMS (ES) m/z: 256.2 [M+H]⁺.

Step-2: Synthesis of tert-butyl N-(3-acetyl-2-fluorophenyl)carbamate (E-157b): Compound E-157b (0.75 g) was synthesized by following procedure as described for the synthesis I-35 (step-5) using tert-butyl N-[2-fluoro-3-(1-hydroxyethyl)phenyl]carbamate (157a) (1.0 g, 3.92 mmol) as the starting material. LCMS (ES) m/z: 154.2 [(M-Boc)+H]⁺.

Step-3: Synthesis of tert-butyl N-[3-(1-aminoethyl)-2-fluorophenyl]carbamate (E-157c): Compound 157c (0.4 g) was synthesized by following procedure as described for the synthesis of I-53 (step-2) using 1 tert-butyl N-(3-acetyl-2-fluorophenyl)carbamate (157b) (0.75 g, 2.96 mmol) as the starting material. LCMS (ES) m/z: 255.1 [M+H]⁺.

Step-4: Synthesis of tert-butyl N-(2-fluoro-3-{1-[({2-hydroxy-4-[(4-methoxyphenyl) methoxy]phenyl}methyl)amino]ethyl}phenyl)carbamate (E-157d): Compound E-157d (1.0 g) was synthesized by following procedure as described for the synthesis of E-01 (step-1) using 2-hydroxy-4-[(4-methoxyphenyl)methoxy]benzaldehyde (I-01) (0.75 g, 2.9 mmol) and tert-butyl N-[3-(1-aminoethyl)-2-fluorophenyl]carbamate (E-157c) (0.738 g 2.9 mmol) as the starting material. LCMS (ES) m/z: 497.3 [M+H]⁺.

Step-5: Synthesis of tert-butyl N-[2-fluoro-3-(1-{7-[(4-methoxyphenyl)methoxy]-2-oxo-3,4-dihydro-2H-1,3-benzoxazin-3-yl}ethyl)phenyl]carbamate (E-157e): Compound E-157e (0.85 g) was synthesized by following procedure as described for the synthesis of E-01 (step-2) using tert-butyl N-(2-fluoro-3-{1-[({2-hydroxy-4-[(4-methoxyphenyl)methoxy]phenyl}methyl)amino]ethyl}phenyl)carbamate (E-157d) (0.9 g, 1.81 mmol) as the starting material. LCMS (ES) m/z: 467.1 [(M-^{t}Bu)+H]⁺.

Step-6: Synthesis of tert-butyl N-{2-fluoro-3-[1-(7-hydroxy-2-oxo-3,4-dihydro-2H-1,3-benzoxazin-3-yl)ethyl]phenyl}carbamate (E-157f): Compound E-157f (0.3 g) was synthesized by following procedure as described for the synthesis of E-01 (step-3) using tert-butyl N-[2-fluoro-3-(1-{7-[(4-methoxyphenyl)methoxy]-2-oxo-3,4-dihydro-2H-1,3-benzoxazin-3-yl}ethyl)phenyl]carbamate (E-157e) (0.45 g, 0.861 mmol) as the starting material. LCMS (ES) m/z: 347.1 [(M-Bu)+H]⁺.

Step-7: Synthesis of 3-[1-(3-{[(tert-butoxy)carbonyl]amino}-2-fluorophenyl)ethyl]-2-oxo-3,4-dihydro-2H-1,3-benzoxazin-7-yl N,N-dimethylcarbamate (E-157g): Compound E-157g (0.4 g) was synthesized by following procedure as described for the synthesis of E-01 (step-4) using 1 tert-butyl N-{2-fluoro-3-[1-(7-hydroxy-2-oxo-3,4-dihydro-2H-1,3-benzoxazin-3-yl)ethyl]phenyl}carbamate (E-157f) (0.45 g, 1.12 mmol) as the starting material. LCMS (ES) m/z: 418.0 [(M-^{t}Bu)+H]⁺.

Step-8: Synthesis of 3-[1-(3-amino-2-fluorophenyl)ethyl]-2-oxo-3,4-dihydro-2H-1,3-benzoxazin-7-yl N,N-dimethylcarbamate (E-157h): Compound E-157h (0.3 g) was synthesized by following procedure as described for the synthesis of E-01 (step-5) using 3-[1-(3-{[(tert-butoxy)carbonyl]amino}-2-fluorophenyl)ethyl]-2-oxo-3,4-dihydro-2H-1,3-benzoxazin-7-yl N,N-dimethylcarbamate (E-157g) (0.4 g, 0.845 mmol) as the starting material. LCMS (ES) m/z: 374.2 [M+H]⁺.

Step-9: Synthesis of 3-(1-{2-fluoro-3-[(methylsulfamoyl)amino]phenyl}ethyl])-2-oxo-3,4-dihydro-2H-1,3-benzoxazin-7-yl N,N-dimethylcarbamate (E-155): Example E-155 (0.09 g) was synthesized by following procedure as described for the synthesis of E-01 (step-6) using 3-(1-{2-fluoro-3-[(methylsulfamoyl)amino]phenyl}ethyl)-2-oxo-3,4-dihydro-2H-1,3-benzoxazin-7-yl N,N-dimethylcarbamate (E-157h) (0.3 g, 0.803 mmol) as the starting material. LCMS (ES) m/z: 467.2 [M−H]⁺. ¹H NMR (400 MHz, DMSO-d₆): 39.37 (s, 1H); 7.43 (t, J=6.4 Hz, 1H); 7.30-7.10 (m, 4H); 6.90-6.85 (m, 2H); 5.72 (q, J=7.2 Hz, 1H); 4.50 (d, J=14.8 Hz, 1H); 4.00 (d, J=14.8 Hz, 1H); 3.02 (s, 3H); 2.90 (s, 3H); 2.45 (d, J=5.2 Hz, 3H); 1.60 (d, J=6.8 Hz, 3H).

Step-10: Synthesis of E-156 and E-157: Example E-156 (0.016 g) and E-157 (0.017 g) were obtained by enantiomer separation of E-155 by chiral HPLC purification [using CHIRALPAK IA (100 mm×4.6 mm, 3 μm particle size) column, n-Hexane: IPA with 0.1% DEA (70:30) as the mobile phase with 1.0 mL/min flow rate]. Note: Absolute configuration of both the enantiomers was not determined.

Example 156: LCMS (ES) m/z: 467.3[M+H]⁺. ¹H NMR (400 MHz, DMSO-d₆): δ 9.37 (s, 1H); 7.43 (t, J=6.0 Hz, 1H); 7.32-7.18 (m, 4H); 6.88-6.82 (m, 2H); 5.72 (q, J=6.8 Hz, 1H); 4.50 (d, J=14.8 Hz, 1H); 4.00 (d, J=14.8 Hz, 1H); 3.02 (s, 3H); 2.90 (s, 3H); 2.46 (d, J=4.4 Hz, 3H); 1.64 (d, J=7.2 Hz, 3H).

Example 157: LCMS (ES) m/z: 467.2 [M+H]⁺. ¹H NMR (400 MHz, DMSO-d₆): δ 9.35 (s, 1H); 7.43 (t, J=7.6 Hz, 1H); 7.30-7.14 (m, 4H); 6.88-6.82 (m, 2H); 5.72 (q, J=7.2 Hz, 1H); 4.50 (d, J=14.8 Hz, 1H); 4.00 (d, J=14.8 Hz, 1H); 3.02 (s, 3H); 2.90 (s, 3H); 2.46 (d, J=4.4 Hz, 3H); 1.64 (d, J=6.8 Hz, 3H).

### Examples 158 and 159: Preparations of (R)-(N-3-fluoro-4-(1-(2-oxo-7-(pyrimidin-2-yloxy)-2H-benzo[e][1,3]oxazin-3(4H)-yl)ethyl)-N′-methylsulfamide and (S)-(N-3-fluoro-4-(1-(2-oxo-7-(pyrimidin-2-yloxy)-2H-benzo[e][1,3]oxazin-3(4H)-yl)ethyl)-N′-methylsulfamide (E-158 and E-159; Compounds 141 and 140, respectively)

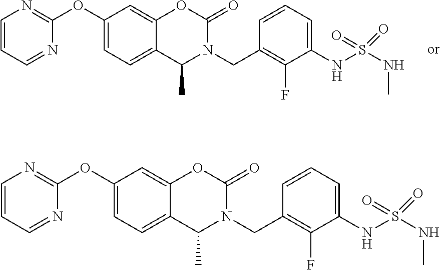 [SMILES: CNS(=O)(=O)Nc1cccc(CN2C(=O)Oc3cc(Oc4ncccn4)ccc3[C@@H]2C)c1F ; CNS(=O)(=O)Nc1cccc(CN2C(=O)Oc3cc(Oc4ncccn4)ccc3[C@H]2C)c1F]

E-158 and E-159 were synthesized by following similar synthesis sequence and experimental procedures as described for the synthesis of E-155, using key intermediates I-01 and E-157c. (note: 2-bromopyrimidine was used in step-7; enantiomers were resolved after step-7 and individual enantiomers were carried forward). Note: Absolute configuration of both the enantiomers was not determined.

E-158: LCMS (ES) m/z: 474.3 [M+H]⁺. ¹H NMR (400 MHz, DMSO-d₆) δ 9.38 (s, 1H); 8.65 (d, J=4.8 Hz, 2H); 7.48-7.40 (m, 1H); 7.32-7.14 (m, 5H); 7.00-6.94 (m, 2H); 5.73 (q, J=7.2 Hz, 1H); 4.54 (d, J=14.8 Hz, 1H); 4.04 (d, J=13.6 Hz, 1H); 2.47 (d, J=4.0 Hz, 3H); 1.62 (d, J=6.8 Hz, 3H).

E-159: LCMS (ES) m/z: 474.3 [M+H]⁺. ¹H NMR (400 MHz, DMSO-d₆) δ 9.38 (s, 1H); 8.65 (d, J=4.8 Hz, 2H); 7.48-7.40 (m, 1H); 7.32-7.14 (m, 5H); 7.00-6.94 (m, 2H); 5.73 (q, J=7.2 Hz, 1H); 4.54 (d, J=14.8 Hz, 1H); 4.04 (d, J=13.6 Hz, 1H); 2.47 (d, J=4.0 Hz, 3H); 1.62 (d, J=6.8 Hz, 3H).

### Example 160: Preparation of 3-{[3-fluoro-2-(methylaminosulfonylamino)-4-pyridyl]methyl}-7-(2-pyrimidinyloxy)-2H,3H-spiro[1,3-benzoxazine-4,1′-cyclobutan]-2-one. (E-160; Compound 160)

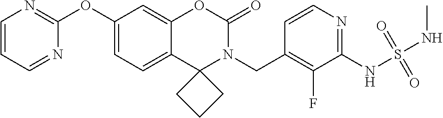 [SMILES: CNS(=O)(=O)Nc1nccc(CN2C(=O)Oc3cc(Oc4ncccn4)ccc3C23CCC3)c1F]

E-160 was synthesized by following similar synthesis sequence and experimental procedures as described for the synthesis of E-130, using key intermediates I-39 and I-35. LCMS (ES) m/z: 501.3 [M+H]⁺. ¹H NMR (400 MHz, DMSO-d₆): δ 10.43 (s, 1H); 8.68 (d, J=4.8 Hz, 2H); 8.00 (d, J=5.2 Hz, 1H); 7.79 (d, J=8.4 Hz, 1H); 7.32 (t, J=4.8 Hz, 1H); 7.18-7.15 (m, 1H); 7.07 (d, J=2.4 Hz, 1H); 7.02-7.00 (m, 1H); 6.80-6.76 (m, 1H); 5.01 (s, 2H); 2.86-2.78 (m, 2H); 2.52 (s, 3H); 2.43-2.38 (m, 2H); 2.02-1.98 (m, 2H).

### Example 161: Synthesis of (cyclopropylsulfonyl)[3-fluoro-4-({2-oxo-7-(1,2,4-triazin-3-yloxy)-2H,3H-spiro[1,3-benzoxazine-4,1′-cyclobutan]-3-yl}methyl)-2-pyridyl]amine. (E-161; Compound 161)

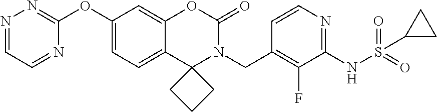 [SMILES: O=C1Oc2cc(Oc3nccnn3)ccc2C2(CCC2)N1Cc1ccnc(NS(=O)(=O)C2CC2)c1F]

E-161 was synthesized by following similar synthesis sequence and experimental procedures as described for the synthesis of E-141, using key intermediates I-39 and I-36. LCMS (ES) m/z: 513.3 [M+H]⁺. ¹H NMR (400 MHz, DMSO-d₆): δ 10.68 (s, 1H); 9.30 (d, J=2.4 Hz, I H); 8.77 (d, J=2.4 Hz, I H); 8.00 (s, 1H); 7.84 (d, J=8.8 Hz, 1H); 7.28-7.26 (m, 1H); 7.19 (d, J=2.0 Hz, 1H); 6.80 (s, 1H); 5.02 (s, 2H); 3.20-3.12 (m, 1H); 2.90-2.80 (m, 2H); 2.44-2.38 (m, 2H); 2.06-1.96 (m, 2H); 1.02-0.98 (m, 4H).

### Example 162: Preparation of 3-{[3-fluoro-2-(methylaminosulfonylamino)-4-pyridyl]methyl}-7-(1,3-oxazol-2-yloxy)-3,4-dihydro-2H-1,3-benzoxazin-2-one. (E-162; Compound 162)

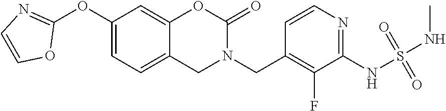 [SMILES: CNS(=O)(=O)Nc1nccc(CN2Cc3ccc(Oc4ncco4)cc3OC2=O)c1F]

E-162 was synthesized by following similar synthesis sequence and experimental procedures as described for the synthesis of E-34, using key intermediates 2,4-dihydroxybenzaldehyde and I-38. LCMS (ES) m/z: 450.3 [M+H]⁺; ¹H NMR (400 MHz, DMSO-d₆): δ 10.42 (s, 1H); 8.05 (d, J=5.2 Hz, 1H); 7.83 (d, J=0.8 Hz, 1H); 7.33 (d, J=8.4 Hz, 1H); 7.22-7.16 (m, 2H); 7.09-7.01 (m, 3H); 4.70 (s, 2H); 4.57 (s, 2H); 2.53 (d, J=4.8 Hz, 3H).

### Example 163: Preparation of (cyclopropylsulfonyl)[3-fluoro-4-({2-oxo-7-(3-pyridazinyloxy)-2H,3H-spiro[1,3-benzoxazine-4,1′-cyclobutan]-3-yl}methyl)-2-pyridyl]amine. (E-163; Compound 163)

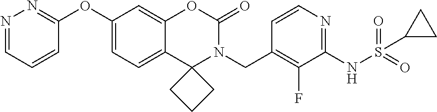 [SMILES: O=C1Oc2cc(Oc3cccnn3)ccc2C2(CCC2)N1Cc1ccnc(NS(=O)(=O)C2CC2)c1F]

E-163 was synthesized by following similar synthesis sequence and experimental procedures as described for the synthesis of E-146, using key intermediates I-36 and I-39. LCMS (ES) m/z: 512.3 [M+H]⁺. ¹H NMR (400 MHz, DMSO-d₆): δ 10.66 (s, 1H); 9.06-9.04 (m, 1H); 8.05 (d, J=4.0 Hz, 1H); 7.84-7.78 (m, 2H); 7.55-7.52 (m, 1H); 7.19-7.16 (m, 1H); 7.08 (d, J=2.4 Hz, 1H); 6.86 (s, 1H); 5.03 (s, 2H); 3.21 (bs, 1H); 2.89-2.79 (m, 2H); 2.44-2.39 (m, 2H); 2.06-1.94 (m, 2H); 1.12-1.00 (m, 4H).

### Example 164: Preparation of 3-{[3-fluoro-2-(methylaminosulfonylamino)-4-pyridyl]methyl}-7-(1,2,4-triazin-3-yloxy)-3,4-dihydro-2H-1,3-benzoxazin-2-one. (E-164; Compound 164)

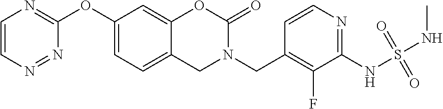 [SMILES: CNS(=O)(=O)Nc1nccc(CN2Cc3ccc(Oc4nccnn4)cc3OC2=O)c1F]

E-164 was synthesized by following similar synthesis sequence and experimental procedures as described for the synthesis of E-27, using key intermediates 2,4-dihydroxybenzaldehyde and I-38. LCMS (ES) m/z: 462.3 [M+H]⁺. ¹H NMR (400 MHz, DMSO-d₆): (10.42 (s, 1H); 9.28 (d, J=2.0 Hz, 1H); 8.75 (d, J=2.4 Hz, 1H); 8.05 (bs, 1H); 7.35 (d, J=8.0 Hz, 1H); 7.15-6.92 (m, 4H); 4.71 (s, 2H); 4.59 (s, 2H); 2.50 (d, J=4.8 Hz, 3H).

### Example 165: Preparation of 6-fluoro-3-{[3-fluoro-2-(methylaminosulfonylamino)-4-pyridyl]methyl}-7-(1,3-oxazol-2-yloxy)-2H,3H-spiro[1,3-benzoxazine-4,1′-cyclobutan]-2-one. (E-165; Compound 165)

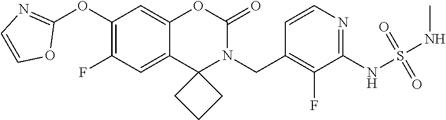 [SMILES: CNS(=O)(=O)Nc1nccc(CN2C(=O)Oc3cc(Oc4ncco4)c(F)cc3C23CCC3)c1F]

E-165 was synthesized by following similar synthesis sequence and experimental procedures as described for the synthesis of E-130, using key intermediates I-41 and I-35. LCMS (ES) n/z: 508.3 [M+H]⁺. ¹H NMR (400 MHz, DMSO-d₆): δ 10.42 (s, 1H); 7.99 (d, J=5.2 Hz, 1H); 7.89-7.86 (m, 2H); 7.54 (d, J=6.8 Hz, 1H); 7.05 (s, 1H); 7.02-6.98 (m, 1H); 6.77 (t, J=5.2 Hz, 1H); 5.00 (s, 2H); 2.83-2.75 (m, 2H); 2.52 (d, J=4.8 Hz, 3H); 2.42-2.32 (m, 2H); 2.04-1.92 (m, 2H).

### Example 166: Preparation of 6-fluoro-3-{[3-fluoro-2-(methylaminosulfonylamino)-4-pyridyl]methyl}-7-(1,2,4-triazin-3-yloxy)-2H,3H-spiro[1,3-benzoxazine-4,1′-cyclobutan]-2-one. (E-166; Compound 166)

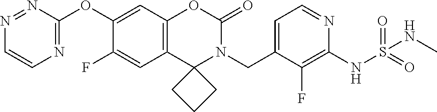 [SMILES: CNS(=O)(=O)Nc1nccc(CN2C(=O)Oc3cc(Oc4nccnn4)c(F)cc3C23CCC3)c1F]

E-166 was synthesized by following similar synthesis sequence and experimental procedures as described for the synthesis of E-130, using key intermediates I-41 and I-35. LCMS (ES) m/z: 520.3 [M+H]⁺. ¹H NMR (400 MHz, DMSO-d₆): δ 10.44 (bs, 1H); 9.37 (d, J=2.0 Hz, 1H); 8.84 (d, J=2.4 Hz, 1H); 7.92-7.80 (m, 2H); 7.44 (d, J=6.8 Hz, 1H); 6.78-6.63 (m, 2H); 4.99 (s, 2H); 2.86-2.78 (m, 2H); 2.47 (d, J=4.8 Hz, 3H); 2.41-2.39 (m, 2H); 2.10-1.90 (m, 2H).

### Example 167: Preparation of (cyclopropylsulfonyl)[3-fluoro-4-({7-(1,3-oxazol-2-yloxy)-2-oxo-2H,3H-spiro[1,3-benzoxazine-4,1′-cyclobutan]-3-yl}methyl)-2-pyridyl]amine. (E-167; Compound 167)

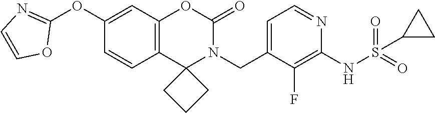 [SMILES: O=C1Oc2cc(Oc3ncco3)ccc2C2(CCC2)N1Cc1ccnc(NS(=O)(=O)C2CC2)c1F]

E-167 was synthesized by following similar synthesis sequence and experimental procedures as described for the synthesis of E-142, using key intermediates I-36 and I-39. LCMS (ES) m/z: 501.3 (M+H)+. ¹H NMR (400 MHz, DMSO-d₆): δ 10.69 (s, 1H); 7.86-7.80 (m, 3H); 7.32 (d, J=8.4 Hz, 1H); 7.27 (s, 1H); 7.07 (s, 1H); 6.50 (bs, 1H); 4.96 (s, 2H); 3.12-3.08 (m, 1H); 2.85-2.77 (m, 2H); 2.38-2.30 (m, 2H); 2.02-1.90 (m, 2H); 1.02-0.80 (m, 4H).

### Example 168: Preparation of 5-fluoro-3-{[3-fluoro-2-(methylaminosulfonylamino)-4-pyridyl]methyl}-7-(1,3-oxazol-2-yloxy)-2H,3H-spiro[1,3-benzoxazine-4,3′-oxetan]-2-one. (E-168; Compound 168)

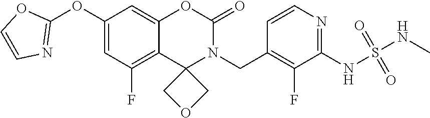 [SMILES: CNS(=O)(=O)Nc1nccc(CN2C(=O)Oc3cc(Oc4ncco4)cc(F)c3C23COC3)c1F]

E-168 was synthesized by following similar synthesis sequence and experimental procedures as described for the synthesis of E-97, using key intermediates I-29 and I-35. LCMS (ES) m/z: 510.3 (M+H)+. ¹H NMR (400 MHz, DMSO-d₆): δ 10.44 (s, 1H); 7.99 (s, 1H); 7.89 (s, 1H); 7.45 (d, J=12.0, Hz, 1H); 7.21-7.14 (m, 2H); 7.10 (s, 1H); 7.03-6.99 (m, 1H); 5.11-5.09 (m, 4H); 4.90-4.86 (m, 2H); 2.51 (d, J=4.8 Hz, 3H).

### Example 169: Preparation of 5-fluoro-3-{[3-fluoro-2-(methylaminosulfonylamino)-4-pyridyl]methyl}-7-(1,3-oxazol-2-yloxy)-2H,3H-spiro[1,3-benzoxazine-4,1′-cyclobutan]-2-one. (E-169; Compound 169)

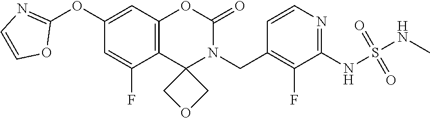 [SMILES: CNS(=O)(=O)Nc1nccc(CN2C(=O)Oc3cc(Oc4ncco4)cc(F)c3C23COC3)c1F]

E-169 was synthesized by following similar synthesis sequence and experimental procedures as described for the synthesis of E-130, using key intermediates I-56 and I-35. LCMS (ES) m/z: 508.4 [M+H]⁺. ¹H NMR (400 MHz, DMSO-d₆): δ 10.45 (s, 1H); 8.01 (s, 1H); 7.89 (s, 1H); 7.40 (d, J=12.4 Hz, 1H); 7.11 (d, J=13.6 Hz, 2H); 7.02-6.96 (m, 2H); 4.94 (s, 2H); 2.80-2.70 (m, 4H); 2.53 (d, J=4.8 Hz, 3H); 2.10-2.00 (m, 2H).

### Example 170: Preparation of (cyclopropylsulfonyl)[3-fluoro-4-({6-fluoro-7-(1,3-oxazol-2-yloxy)-2-oxo-2H,3H-spiro[1,3-benzoxazine-4,1′-cyclobutan]-3-yl}methyl)-2-pyridyl]amine. (E-170; Compound 170)

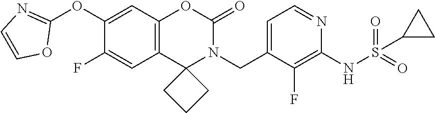 [SMILES: O=C1Oc2cc(Oc3ncco3)c(F)cc2C2(CCC2)N1Cc1ccnc(NS(=O)(=O)C2CC2)c1F]

E-170 was synthesized by following similar synthesis sequence and experimental procedures as described for the synthesis of E-142, using key intermediates I-41 and I-36. LCMS (ES) m/z: 519.4 [M+H]⁺. ¹H NMR (400 MHz, DMSO-d₆): δ 10.70 (s, 1H); 8.00 (s, 1H); 7.90-7.87 (m, 2H); 7.54 (d, J=6.8 Hz, 1H); 7.05 (d, J=0.8 Hz, 1H); 6.81 (s, 1H); 5.00 (s, 2H); 3.20-3.14 (s, 1H); 2.84-2.76 (m, 2H); 2.42-2.38 (m, 2H); 2.10-1.90 (m, 2H); 1.11-1.00 (m, 4H).

### Example 171: Synthesis of 5-fluoro-3-{[3-fluoro-2-(methylaminosulfonylamino)-4-pyridyl]methyl}-7-(3-pyridazinyloxy)-2H,3H-spiro[1,3-benzoxazine-4,1′-cyclobutan]-2-one. (E-171; Compound 171)

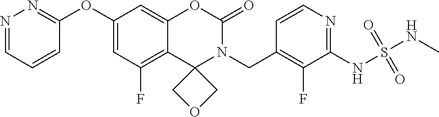 [SMILES: CNS(=O)(=O)Nc1nccc(CN2C(=O)Oc3cc(Oc4cccnn4)cc(F)c3C23COC3)c1F]

E-171 was synthesized by following similar synthesis sequence and experimental procedures as described for the synthesis of E-128, using key intermediates I-55 and I-35. LCMS (ES) m/z: 519.4 [M+H]⁺. ¹H NMR (400 MHz, DMSO-d₆): δ 10.45 (s, 1H); 9.08 (d, J=4.0 Hz, 1H); 8.01 (s, 1H); 7.86-7.82 (m, 1H); 7.58 (d, J=8.8 Hz, 1H); 7.22 (d, J=12.4 Hz, 1H); 7.06-6.98 (m, 2H); 6.94 (s, 1H); 4.95 (s, 2H); 2.81-2.75 (m, 4H); 2.54 (d, J=4.8 Hz, 3H); 2.10-2.00 (m, 2H).

### Example 172: Synthesis of N-(3-fluoro-4-((6-fluoro-7-(oxazol-2-yloxy)-2-oxospiro[benzo[e][1,3]oxazine-4,3′-oxetan]-3(2H)-yl)methyl)pyridin-2-yl)cyclopropanesulfonamide. (E-172; Compound 172)

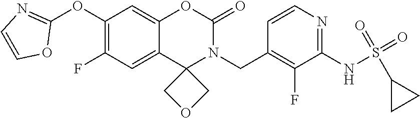 [SMILES: O=C1Oc2cc(Oc3ncco3)c(F)cc2C2(COC2)N1Cc1ccnc(NS(=O)(=O)C2CC2)c1F]

E-172 was synthesized by following similar synthesis sequence and experimental procedures as described for the synthesis of E-132, using key intermediates I-28 and I-36. LCMS (ES) m/z: 521.4 [M+H]⁺. ¹H NMR (400 MHz, DMSO-d₆): δ 10.67 (s, 1H); 8.04-7.94 (m, 2H); 7.88 (d, J=0.8 Hz, 1H); 7.52 (d, J=6.8 Hz, 1H); 7.05 (s, 1H); 7.10-7.00 (m, 1H); 5.10-5.04 (m, 4H); 4.83 (d, J=8.0 Hz, 2H); 3.20-3.16 (m, 1H); 1.10-1.00 (m, 4H).

### Example 173: Synthesis of 8-fluoro-3-{[3-fluoro-2-(methylaminosulfonylamino)-4-pyridyl]methyl}-7-(1,3-oxazol-2-yloxy)-2H,3H-spiro[1,3-benzoxazine-4,1′-cyclobutan]-2-one (E-173; Compound 173)

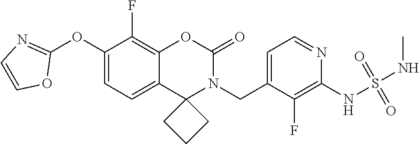 [SMILES: CNS(=O)(=O)Nc1nccc(CN2C(=O)Oc3c(ccc(Oc4ncco4)c3F)C23CCC3)c1F]

E-173 was synthesized by following similar synthesis sequence and experimental procedures as described for the synthesis of E-130, using key intermediates I-35 and I-56. LCMS (ES) m/z: 508.4 [M+H]⁺. ¹H NMR (400 MHz, DMSO-d₆): δ 10.43 (s, 1H); 7.97 (d, J=3.6 Hz, 1H); 7.89 (s, 1H); 7.66 (d, J=8.8 Hz, 1H); 7.54-7.50 (m, 1H); 7.06 (s, 1H); 6.97 (bs, 1H); 6.81 (bs, 1H); 5.02 (s, 2H); 2.88-2.80 (m, 2H); 2.50 (d, J=4.8 Hz, 3H); 2.42-2.32 (m, 2H); 2.02-1.98 (m, 2H).

### Example 174: Synthesis of 8-fluoro-3-{[3-fluoro-2-(methylaminosulfonylamino)-4-pyridyl]methyl}-7-(3-pyridazinyloxy)-2H,3H-spiro[1,3-benzoxazine-4,1′-cyclobutan]-2-one (E-174; Compound 174)

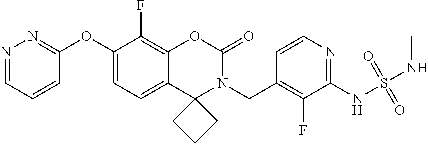 [SMILES: CNS(=O)(=O)Nc1nccc(CN2C(=O)Oc3c(ccc(Oc4cccnn4)c3F)C23CCC3)c1F]

E-174 was synthesized by following similar synthesis sequence and experimental procedures as described for the synthesis of E-128, using key intermediates I-35 and I-56. LCMS (ES) m/z: 519.4 [M+H]⁺. ¹H NMR (400 MHz, DMSO d₆): δ 10.44 (s, 1H); 9.08 (d, J=4.4 Hz 1H); 8.01 (d, J=4.4 Hz, 1H); 7.88-7.84 (m, 1H); 7.70-7.64 (m, 2H); 7.36 (t, J=7.6 Hz, 1H); 7.01 (bs, 1H); 6.85 (bs, 1H); 5.04 (s, 2H); 2.90-2.80 (m, 2H); 2.52 (d, J=4.8 Hz, 3H); 2.50-2.40 (m, 2H); 2.04-1.96 (m, 2H).

Example 175 and Example 176: Synthesis of 3-{[2-fluoro-3-(methylaminosulfonylamino)phenyl]methyl}-4-methyl-7-(3-pyridazinyloxy)-3,4-dihydro-2H-1-oxa-3,5-diazanaphthalen-2-one (E-175 and E-176)

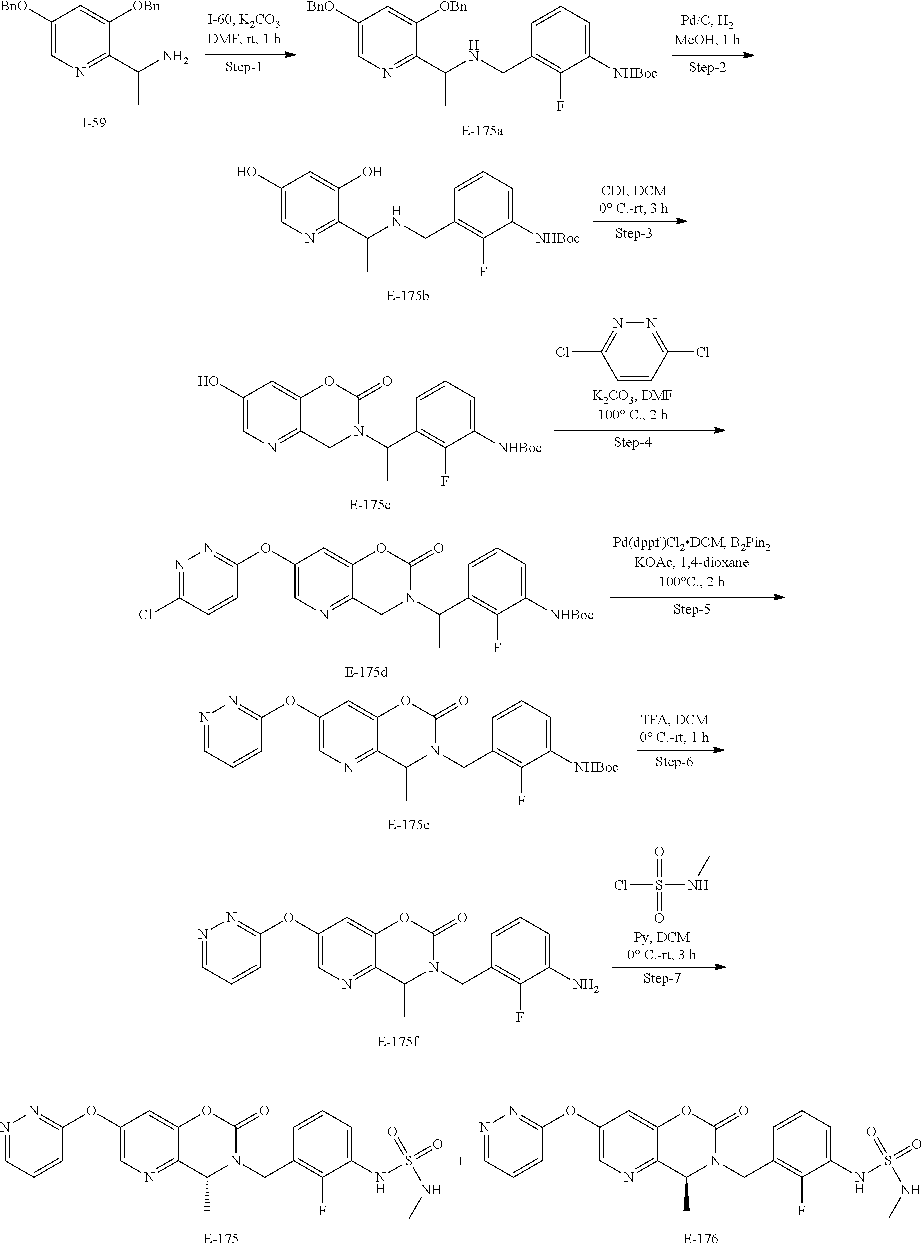

Step-1: Synthesis of tert-butyl (3-(((1-(3,5-bis(benzyloxy)pyridin-2-yl)ethyl)amino)methyl)-2-fluorophenyl)carbamate (E-175a): To a stirred solution of 1-[3,5-bis(benzyloxy)-2-pyridyl]ethylamine (I-59) (3 g, 8.97 mmol) in DMF (60 mL, 775 mmol) was added K₂CO₃ (2.48 g, 17.9 mmol) and 3-(bromomethyl)-2-fluorophenylamino-tert-butylformylate (I-60) (3.27 g, 10.8 mmol) at room temperature. The reaction mixture was stirred at room temperature for 1 h, while monitoring reaction progress by TLC. After complete consumption of starting material (1 h), 100 mL water was added to the reaction mixture and extraction was carried out using EtOAc (50 mL×3). The combined organic extracts were washed with brine solution (10 mL), dried over anhydrous Na₂SO₄, filtered and concentrated under reduced pressure. The residue was purified by silica gel column chromatography (using gradient elution of 0-30% EtOAc in heptane) to afford desired compound (E-175a) (4.4 g) as a yellow semi-solid. LCMS (ES) m/z: 558.25 [M+H]⁺.

Step-2: Synthesis of tert-butyl (3-(((1-(3,5-dihydroxypyridin-2-yl)ethyl)amino)methyl)-2-fluorophenyl)carbamate (E-175b): To a stirred solution of tert-butyl (3-(((1-(3,5-bis(benzyloxy)pyridin-2-yl)ethyl)amino)methyl)-2-fluorophenyl)carbamate (E-175a) (4.4 g, 7.89 mmol) in MeOH (50 mL) was added 10% Pd/C (0.84 g, 7.89 mmol) under nitrogen atmosphere. The reaction mixture was then stirred at room temperature under H₂ atmosphere; while monitoring reaction progress by TLC. After completion of the reaction (1 h), the catalyst was filtered off through celite bed and washed with MeOH (100 mL). The filtrate was concentrated under reduced pressure to afford desired compound E-175b (2.25 g) as an off white solid. LCMS (ES) m/z: 378.20 [M+H]⁺.

Step-3: Synthesis of tert-butyl (2-fluoro-3-((7-hydroxy-4-methyl-2-oxo-2H-pyrido[2,3-e][1,3]oxazin-3(4H)-yl)methyl)phenyl)carbamate (E-175c): To a stirred solution of tert-butyl (3-(((1-(3,5-dihydroxypyridin-2-yl)ethyl)amino)methyl)-2-fluorophenyl)carbamate (E-175b) (2.2 g, 5.83 mmol) in THF (20 mL) was added CDi (1.42 g, 8.74 mmol) at 0° C. and the reaction mixture was stirred at room temperature for 3 h. Volatiles were then removed under reduced pressure and the residue was purified by silica gel column chromatography (using gradient elution of 0-10% MeOH in DCM) to afford desired compound (E-175c) (1.3 g) as off white solid. LCMS (ES) m/z: 404.20 [M+H]⁺.

Step-4: Synthesis of tert-butyl (3-((7-((6-chloropyridazin-3-yl)oxy)-4-methyl-2-oxo-2H-pyrido[2,3-e][1,3]oxazin-3(4H)-yl)methyl)-2-fluorophenyl)carbamate (E-175d): To a stirred solution of tert-butyl (2-fluoro-3-((7-hydroxy-4-methyl-2-oxo-2H-pyrido[2,3-e][1,3]oxazin-3(4H) yl)methyl)phenyl)carbamate (E-175c) (1.3 g, 3.22 mmol) in DMF (10 mL) was added potassium carbonate (0.891 g, 6.45 mmol) and 3,6-dichloropyridazine (0.48 g, 3.22 mmol) at room temperature. Then the reaction mixture was stirred at 100° C. for 2 h. After complete consumption of starting material (2 h), the reaction mixture was cooled to room temperature and ice cold water (15 mL) was added to it. The solid obtained was collected by vacuum filtration and washed with water. The crude was further purified by Combi-Flash (using gradient elution of 0-7% of MeOH in DCM) to afford the desired compound (E-175d) (1.1 g) as brown solid. LCMS (ESI) m/z: 514.10 [M−H]⁺.

Step-5: Synthesis of tert-butyl (2-fluoro-3-((4-methyl-2-oxo-7-(pyridazin-3-yloxy)-2H-pyrido[2,3-e][1,3]oxazin-3(4H)-yl)methyl)phenyl)carbamate (E-175e): Argon gas was purged through a stirred suspension of tert-butyl (3-((7-((6-chloropyridazin-3-yl)oxy)-4-methyl-2-oxo-2H-pyrido[2,3-e][1,3]oxazin-3(4H)-yl)methyl)-2-fluorophenyl)carbamate (E-175d) (1.1 g, 2.13 mmol), potassium acetate (0.628 g, 6.4 mmol) and bis-pinacolatodiborane (0.596 g, 2.35 mmol) in 1,4-dioxane (8.6 mL) for 5 min before addition of PdCl₂(dppf). DCM (0.174 g, 0.213 mmol). The reaction mixture was then stirred at 100° C. for 2 h. After complete consumption of starting material (2 h), the reaction mixture was cooled to room temperature and water (30 mL) was added to it. Extraction was carried out using EtOAc (2×30 mL); the combined organic extracts were washed with brine (30 mL), dried over anhydrous sodium sulfate, filtered and concentrated under reduced pressure. The residue was further purified by Combi-Flash (using gradient elution of 0-20% of MeOH in DCM) to afford desired compound (E-175e) (0.71 g) as off white solid. LCMS (ES) m/z: 482.2 [M+H]⁺.

Step-6: Synthesis of 3-(3-amino-2-fluorobenzyl)-4-methyl-7-(pyridazin-3-yloxy)-3,4-dihydro-2H-pyrido[2,3-e][1,3]oxazin-2-one (E-175f): To a stirred solution of tert-butyl (2-fluoro-3-((4-methyl-2-oxo-7-(pyridazin-3-yloxy)-2H-pyrido[2,3-e][1,3]oxazin-3(4H)-yl)methyl)phenyl)carbamate (E-175e)) (0.70 g, 1.45 mmol) in DCM (10 mL) was added trifluoroacetic acid (0.663 mL, 5.82 mmol) at 0° C. The reaction mixture was stirred at room temperature for 1 h. After completion of the reaction, volatiles were removed under reduced pressure and saturated solution of NaHCO₃ (30 mL) was added to the residue. Extraction was carried out using EtOAc (3×30 mL); the combined organic extracts were dried over Na₂SO₄, filtered and concentrated under reduced pressure to afford desired compound (E-175f) (0.38 g) as an off white solid. LCMS (ES) m/z: 382.20 [M+H]⁺.

Step-7: Synthesis of (R)-3-{[2-fluoro-3-(methylaminosulfonylamino)phenyl]methyl}-4-methyl-7-(3-pyridazinyloxy)-3,4-dihydro-2H-1-oxa-3,5-diazanaphthalen-2-one (E-175): To a solution of 3-(3-amino-2-fluorobenzyl)-4-methyl-7-(pyridazin-3-yloxy)-3,4-dihydro-2H-pyrido[2,3-e][1,3]oxazin-2-one (E-175f) (0.38 g, 0.996 mmol) in DCM (5 mL) was added pyridine (2 mL, 3.03 mmol) and methylsulfamoylchlrodie (0.166 g, 1.49 mmol) at 0° C. The reaction mixture was stirred at room temperature for 3 h, while monitoring reaction progress by TLC/LCMS. After completion of the reaction (3 h), ice water (20 mL) was added to it and extraction was carried out using EtOAc (2×30 mL). The combined organic extract was washed with brine solution (30 mL), dried over anhydrous Na₂SO₄, filtered and concentrated under reduced pressure. The residue was purified by Combi-Flash (using gradient elution of 0-7% of MeOH in DCM) to afford desired compound as a racemic mixture. Enantiomers were then separated by chiral SFC [CHIRALPAK AD-H (20.0 mm I.D*250m mL) 5u using CO₂: 100% MeOH (70:30) using SFC purification method]. The resolved enantiomers were further purified by reverse phase preparative HPLC [using Triat YMC (19×250 mm), 10 μm and acetonitrile: water as mobile Phase with 18 mL/min flow rate] to afford desired compounds.

E-175 (0.05 g) as an off-white solid: LCMS (ESI) m/z: 475.20 [M+H]⁺. ¹H NMR (400 MHz, DMSO-d₆): δ 9.44 (s, 1H); 9.06 (d, J=4.4 Hz, 1H); 8.36 (d, J=2.4 Hz, 1H); 7.85-7.81 (m, 1H); 7.71 (d, J=2.41H); 7.62 (dd, J₁=0.8 Hz, J₂=8.8 Hz, 1H); 7.41-7.36 (m, 1H); 7.27-7.22 (m, 1H); 7.18-7.12 (m, 2H); 4.94 (d, J=15.6 Hz, 1H); 4.63 (q, J=6.4 Hz, 1H); 4.57 (d, J=15.6 Hz, 1H); 2.48 (d, J=4.4 Hz, 3H); 1.51 (d, J=6.4 Hz, 3H).

E-176 (0.03 g) as an off-white solid: LCMS (ES) m/z: 475.20 [M+H]⁺. ¹H NMR (400 MHz, DMSO-d₆): δ 9.44 (s, 1H); 9.06 (d, J=4.4 Hz, 1H); 8.36 (d, J=2.4 Hz, 1H); 7.85-7.81 (m, 1H); 7.71 (d, J=2.41H); 7.62 (dd, J₁=0.8 Hz, J₂=8.8 Hz, 1H); 7.41-7.36 (m, 1H); 7.27-7.22 (m, 1H); 7.18-7.12 (m, 2H); 4.94 (d, J=15.6 Hz, 1H); 4.63 (q, J=6.4 Hz, 1H); 4.57 (d, J=15.6 Hz, 1H); 2.48 (d, J=4.4 Hz, 3H); 1.51 (d, J=6.4 Hz, 3H). Note: Absolute configuration of both the enantiomers was not determined.

### Example 177: Synthesis of 8-fluoro-3-{[3-fluoro-2-(methylaminosulfonylamino)-4-pyridyl]methyl}-7-(1,2,4-triazin-3-yloxy)-2H,3H-spiro[1,3-benzoxazine-4,1′-cyclobutan]-2-one (E-177; Compound 177)

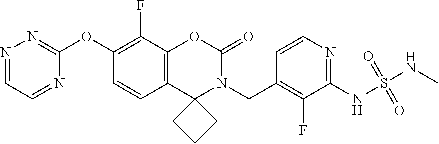 [SMILES: CNS(=O)(=O)Nc1nccc(CN2C(=O)Oc3c(ccc(Oc4nccnn4)c3F)C23CCC3)c1F]

E-177 was synthesized by following similar synthesis sequence and experimental procedures as described for the synthesis of E-130, using key intermediates I-56 and I-35. LCMS (ES) m/z: 520.2 [M+H]⁺. ¹H NMR (400 MHz, DMSO-d₆): δ 10.45 (bs, 1H); 9.37 (d, J=2.4 Hz, 1H); 8.84 (d, J=2.4 Hz, 1H); 7.96 (d, J=4.4 Hz, 1H); 7.68 (dd, J₁=1.2 Hz, J₂=8.8 Hz, 1H); 7.46-7.42 (m, 1H); 6.95 (brs, 1H); 6.81 (bs, 1H); 5.02 (s, 2H); 2.90-2.80 (m, 2H); 2.46 (d, J=4.4 Hz, 3H); 2.48-2.42 (m, 2H); 2.02-1.94 (m, 2H).

### Example 178: Synthesis of 5-fluoro-3-{[3-fluoro-2-(methylaminosulfonylamino)-4-pyridyl]methyl}-7-(3-pyridazinyloxy)-2H, 3H-spiro[1,3-benzoxazine-4,3′-oxetan]-2-one (E-178; Compound 178)

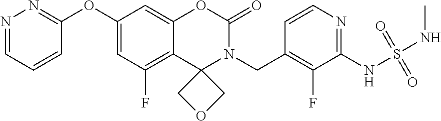 [SMILES: CNS(=O)(=O)Nc1nccc(CN2C(=O)Oc3cc(Oc4cccnn4)cc(F)c3C23COC3)c1F]

E-178 was synthesized by following similar synthesis sequence and experimental procedures as described for the synthesis of E-135, using key intermediates I-29 and I-35. LCMS (ES) m/z: 521.4 [M+H]⁺. ¹H NMR (400 MHz, DMSO-d₆): δ 10.44 (s, 1H); 9.07 (d, J=4.4 Hz, 1H); 7.99 (s, 1H); 7.86-7.82 (m, 1H); 7.58 (d, J=8.8 Hz, 1H); 7.26 (d, J=12.4 Hz, 1H); 7.17 (bs, 1H); 7.02-6.96 (m, 2H); 5.16-5.10 (m, 4H); 4.89 (d, J=8.0 Hz, 2H); 2.50 (d, J=4.4 Hz, 3H).

### Example 179: Synthesis of 3-{[2-(dimethylaminosulfonylamino)-3-fluoro-4-pyridyl]methyl}-7-(1,3-oxazol-2-yloxy)-2H,3H-spiro[1,3-benzoxazine-4,1′-cyclobutan]-2-one (E-179; Compound 179)

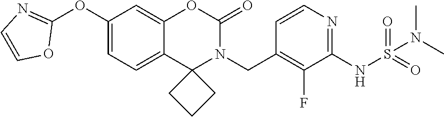 [SMILES: CN(C)S(=O)(=O)Nc1nccc(CN2C(=O)Oc3cc(Oc4ncco4)ccc3C23CCC3)c1F]

E-179 was synthesized by following similar synthesis sequence and experimental procedures as described for the synthesis of E-142, using key intermediates I-39 and I-57. LCMS (ES) m/z: 504.4 [M+H]⁺. ¹H NMR (400 MHz, DMSO-d₆): δ 10.44 (s, 1H); 7.97 (bs, 1H); 7.86 (s, 1H); 7.83 (d, J=8.8 Hz, 1H); 7.33 (d, J=8.8 Hz, 1H); 7.26 (s, 1H); 7.07 (s, 1H), 6.75 (bs, 1H); 5.00 (s, 2H); 2.81 (s, 6H); 2.78-2.68 (m, 2H); 2.40-2.32 (m, 2H); 2.04-1.92 (m, 2H).

### Example 180: Synthesis of 3-{[3-fluoro-2-(methylaminosulfonylamino)-4-pyridyl]methyl}-7-(5-methyl-1,3-oxazol-2-yloxy)-2H,3H-spiro[1,3-benzoxazine-4,1′-cyclobutan]-2-one (E-180; Compound 180)

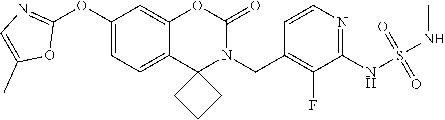 [SMILES: CNS(=O)(=O)Nc1nccc(CN2C(=O)Oc3cc(Oc4ncc(C)o4)ccc3C23CCC3)c1F]

E-180 was synthesized by following similar synthesis sequence and experimental procedures as described for the synthesis of E-130, using key intermediates I-35 and I-39. LCMS (ES) m/z: 504.4 [M+H]⁺. ¹H NMR (400 MHz, DMSO-d₆): δ 10.43 (s, 1H); 7.97 (s, 1H); 7.81 (d, J=8.4 Hz, 1H); 7.29 (d, J=8.8 Hz, 1H); 7.22 (s, 1H); 6.97 (bs, 1H); 6.74-6.66 (m, 2H); 5.00 (s, 2H); 2.85-2.76 (m, 2H); 2.52 (d, J=4.4 Hz, 3H); 2.40-2.32 (m, 2H); 2.26 (s, 3H); 2.02-1.92 (m, 2H).

### Example 181: Synthesis of 3-{[2-(dimethylaminosulfonylamino)-3-fluoro-4-pyridyl]methyl}-7-(3-pyridazinyloxy)-2H,3H-spiro[1,3-benzoxazine-4,1′-cyclobutan]-2-one (E-181; Compound 181)

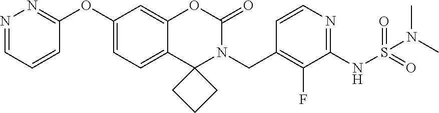 [SMILES: CN(C)S(=O)(=O)Nc1nccc(CN2C(=O)Oc3cc(Oc4cccnn4)ccc3C23CCC3)c1F]

E-181 was synthesized by following similar synthesis sequence and experimental procedures as described for the synthesis of E-146, using key intermediates I-39 and I-57. LCMS (ES) m/z: 515.4 [M+H]⁺. ¹H NMR (400 MHz, DMSO-d₆): δ 10.12 (bs, 1H); 9.04 (d, J=3.6 Hz, 1H); 7.90-7.78 (m, 3H); 7.54 (d, J=8.8 Hz, 1H); 7.17 (d, J=8.4 Hz, 1H); 7.08 (s, 1H); 6.55 (bs, 1H); 4.97 (s, 2H); 2.88-2.78 (m, 2H); 2.75 (s, 6H); 2.42-2.38 (m, 2H); 2.06-1.96 (m, 2H).

### Example 182: Synthesis 3-{[3-fluoro-2-(methylaminosulfonylamino)-4-pyridyl]methyl}-7-(4-methyl-1,3-oxazol-2-yloxy)-2H,3H-spiro[1,3-benzoxazine-4,1′-cyclobutan]-2-one (E-182; Compound 182)

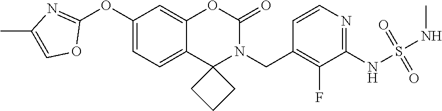

E-182 was synthesized by following similar synthesis sequence and experimental procedures as described for the synthesis of E-130, using key intermediates I-35 and I-39. LCMS (ES) m/z: 504.4 [M+H]⁺; ¹H NMR (400 MHz, DMSO-d₆): δ 10.42 (s, 1H); 7.96 (s, 1H); 7.82 (d, J=8.4 Hz, 1H); 7.56 (s, 1H); 7.32 (d, J=8.4 Hz, 1H); 7.26 (s, 1H); 6.94 (bs, 1H); 6.70 (bs, 1H); 5.00 (s, 2H); 2.85-2.77 (m, 2H); 2.52 (d, J=4.4 Hz, 3H); 2.40-2.32 (m, 2H); 2.02 (s, 3H); 2.01-1.92 (m, 2H).

### Example 183: Synthesis of (cyclopropylsulfonyl)[3-fluoro-4-({8-fluoro-7-(1,3-oxazol-2-yloxy)-2-oxo-2H,3H-spiro[1,3-benzoxazine-4,1′-cyclobutan]-3-yl}methyl)-2-pyridyl]amine (E-183; Compound 183)

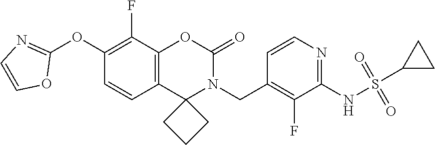 [SMILES: O=C1Oc2c(ccc(Oc3ncco3)c2F)C2(CCC2)N1Cc1ccnc(NS(=O)(=O)C2CC2)c1F]

E-183 was synthesized by following similar synthesis sequence and experimental procedures as described for the synthesis of E-142, using key intermediates I-36 and I-56. LCMS (ESI) m/z: 519.3 [M+H]⁺. ¹H NMR (400 MHz, DMSO-cd₆): δ 10.69 (s, 1H); 8.01 (bs, 1H); 7.89 (d, J=4.8 Hz, 1H); 7.65 (dd, J₁=2.0 Hz, J₂=8.8 Hz, 1H); 7.52 (dd, J₁=7.2 Hz, J₂=8.8 Hz, 1H); 7.06 (d, J=1.2 Hz, 1H); 6.91 (s, 1H); 5.02 (s, 2H); 3.22-3.14 (m, 1H); 2.89-2.79 (m, 2H); 2.41-2.32 (m, 2H); 2.03-1.92 (m, 2H); 1.10-1.00 (m, 4H).

### Example 184: Synthesis of (cyclopropylsulfonyl)[3-fluoro-4-({8-fluoro-2-oxo-7-(3-pyridazinyloxy)-2H,3H-spiro[1,3-benzoxazine-4,1′-cyclobutan]-3-yl}methyl)-2-pyridyl]amine (E-184; Compound 184)

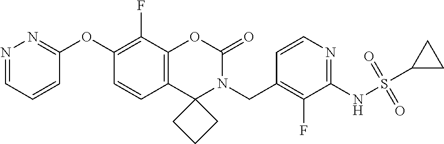 [SMILES: O=C1Oc2c(ccc(Oc3cccnn3)c2F)C2(CCC2)N1Cc1ccnc(NS(=O)(=O)C2CC2)c1F]

E-184 was synthesized by following similar synthesis sequence and experimental procedures as described for the synthesis of E-146, using key intermediates I-36 and I-56. LCMS (ES) m/z: 530.3 [M+H]⁺. ¹H NMR (400 MHz, DMSO-d₆): b 10.71 (s, 1H); 9.07 (dd, J₁=1.2 Hz, J₂=4.4 Hz, 1H); 7.99 (bs, 1H); 7.87.84 (m, 1H); 7.68-7.64 (m, 2H); 7.35 (dd, J₁=7.2 Hz, J₂=8.4 Hz, 1H); 6.87 (bs, 1H); 5.03 (s, 2H); 3.20-3.12 (m, 1H); 2.90-2.80 (m, 2H); 2.46-2.40 (m, 2H); 2.05-1.95 (m, 2H); 1.10-0.96 (m, 4H).

### Example 185: Synthesis of 6-fluoro-3-{[3-fluoro-2-(methylaminosulfonylamino)-4-pyridyl]methyl}-4-methyl-7-(1,3-oxazol-2-yloxy)-3,4-dihydro-2H-1,3-benzoxazin-2-one (E-185; Compound 185)

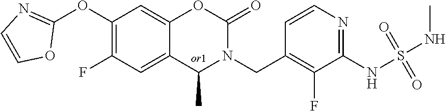

E-185 was synthesized by following similar synthesis sequence and experimental procedures as described for the synthesis of E-135, using key intermediates I-35 and I-58. LCMS (ES) m/z: 482.3 [M+H]⁺. ¹H NMR (400 MHz, DMSO-d₆): δ 10.43 (s, 1H); 8.03 (d, J=4.8 Hz, 1H); 7.86 (d, J=0.8 Hz, 1H); 7.55 (d, J=4.4 Hz, 1H); 7.52 (s, 1H); 7.03 (s, 1H); 7.06-7.00 (m, 1H); 6.97-6.93 (m, 1H); 4.89 (d, J=16.4 Hz, 1H); 4.71 (q, J=6.4 Hz, 1H); 4.57 (d, J=16.4 Hz, 1H); 2.52 (d, J=4.4 Hz, 3H); 1.46 (d, J=6.4 Hz, 3H). Note: absolute configuration was not determined.

### Example 186: Synthesis of 6-fluoro-3-{[3-fluoro-2-(methylaminosulfonylamino)-4-pyridyl]methyl}-4-methyl-7-(1,3-oxazol-2-yloxy)-3,4-dihydro-2H-1,3-benzoxazin-2-one (E-186; Compound 186)

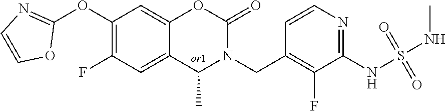

E-186 was synthesized by following similar synthesis sequence and experimental procedures as described for the synthesis of E-97, using key intermediates I-35 and I-58. LCMS (ES) m/z: 482.3 [M+H]⁺. ¹H NMR (400 MHz, DMSO-d₆): δ 10.33 (s, 1H); 8.00 (d, J=4.8 Hz, 1H); 7.86 (d, J=0.8 Hz, 1H); 7.55 (d, J=4.4 Hz, 1H); 7.52 (s, 1H); 7.03 (d, J=0.8 Hz, 1H); 7.00-6.88 (m, 2H); 4.89 (d, J=16.4 Hz, 1H); 4.71 (q, J=6.4 Hz, 1H); 4.57 (d, J=16.4 Hz, 1H); 2.52 (d, J=4.4 Hz, 3H); 1.46 (d, J=6.4 Hz, 3H). Note: absolute configuration was not determined.

### Example 187: Synthesis of (cyclopropylsulfonyl)[3-fluoro-4-({8-fluoro-2-oxo-7-(1,2,4-triazin-3-yloxy)-2H,3H-spiro[1,3-benzoxazine-4,1′-cyclobutan]-3-yl}methyl)-2-pyridyl]amine (E-187; Compound 187)

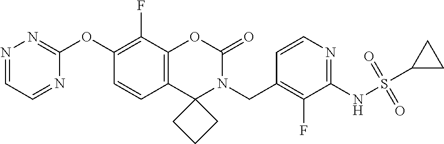 [SMILES: O=C1Oc2c(ccc(Oc3nccnn3)c2F)C2(CCC2)N1Cc1ccnc(NS(=O)(=O)C2CC2)c1F]

E-187 was synthesized by following similar synthesis sequence and experimental procedures as described for the synthesis of E-142, using key intermediates I-36 and I-56. LCMS (ES) n/z: 531.10 [M+H]⁺. ¹H NMR (400 MHz, DMSO-d₆): δ 10.66 (s, 1H); 9.37 (d, J=2.4 Hz, 1H); 8.83 (d, J=2.4 Hz, 1H); 8.03 (bs, 1H); 7.68 (d, J=7.6 Hz, 1H); 7.44 (dd, J₁=7.6 Hz, J_{z}=8.4 Hz, 1H); 6.94 (s, 1H); 5.04 (s, 2H); 3.22-3.14 (m, 1H); 2.90-2.80 (m, 2H); 2.46-2.40 (m, 2H); 2.05-1.95 (m, 2H); 1.10-1.00 (m, 4H).

Example 188 and Example 189: Synthesis of 3-{[2-fluoro-3-(methylaminosulfonylamino)phenyl]methyl}-4-methyl-7-(1,3-oxazol-2-yloxy)-3,4-dihydro-2H-1-oxa-3,5-diazanaphthalen-2-one (E-188 and E-189; Compound 188 and Compound 189, respectively)

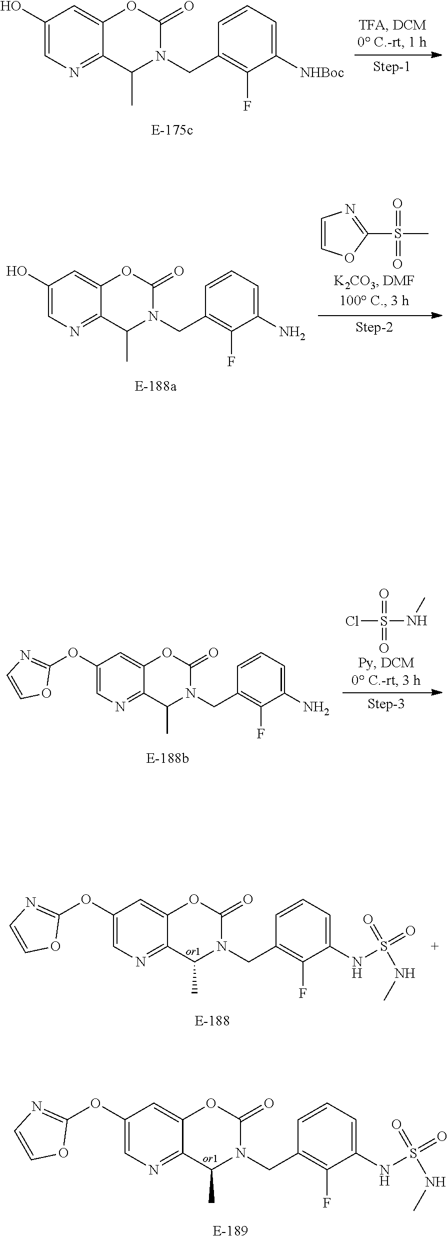

Step-1: Synthesis of 3-(3-amino-2-fluorobenzyl)-7-hydroxy-4-methyl-3,4-dihydro-2H-pyrido[2,3-e][1,3]oxazin-2-one (E-188a): Compound E-188a (1.9 g) was synthesized by following procedure as described for the synthesis of E-175 (step-6) using tert-butyl (2-fluoro-3-((7-hydroxy-4-methyl-2-oxo-2H-pyrido[2,3-e][1,3]oxazin-3(4H) yl)methyl)phenyl) carbamate (E-175c) (2.5 g, 6.2 mmol) as starting material. LCMS (ES) m/z: 304.15 [M+H]⁺.

Step-2: Synthesis of 3-(3-amino-2-fluorobenzyl)-4-methyl-7-(oxazol-2-yloxy)-3,4-dihydro-2H-pyrido[2,3-e][1,3]oxazin-2-one (E-188b): Compound E-188b (0.85 g) was synthesized by following procedure as described for the synthesis of E-175 (step-4) using 3-(3-amino-2-fluorobenzyl)-7-hydroxy-4-methyl-3,4-dihydro-2H-pyrido[2,3-e][1,3]oxazin-2-one (E-188a) (1.9 g, 6.26 mmol) and 2-mesyl-1,3-oxazole (1.29 g, 8.77 mmol) as starting materials. LCMS (ES) m/z: 371.15 [M+H]⁺.

Step-3: Synthesis of 3-{[2-fluoro-3-(methylaminosulfonylamino)phenyl]methyl}-4-methyl-7-(1,3-oxazol-2-yloxy)-3,4-dihydro-2H-1-oxa-3,5-diazanaphthalen-2-one (E-188 & E-189): Compound E-188 was synthesized by following procedure as described for the synthesis of

E-175 (Step-7) using 3-(3-amino-2-fluorobenzyl)-4-methyl-7-(oxazol-2-yloxy)-3,4-dihydro-2H-pyrido[2,3-e][1,3]oxazin-2-one (E-188b) as starting materials. Enantiomers were separated by preparative SFC [using CHIRALPAK IK (21.0 mm×250 mm) 5u; spc-CO₂: 100% MeOH (75:25) as mobile phase with 70 mL/min flow rate]. Note: Absolute configuration of both enantiomers were not determined.

E-188: LCMS (ES) m/z: 464.3 [M+H]⁺. ¹H NMR (400 MHz, DMSO-d₆): δ 9.42 (s, 1H); 8.51 (d, J=2.4 Hz, 1H); 7.89 (d, J=2.4 Hz, 1H); 7.87 (d, J=0.8 Hz, 1H); 7.40-7.35 (m, 1H); 7.24-7.20 (m, 1H); 7.16-7.10 (m, 2H); 7.06 (s, 1H); 4.92 (d, J=15.6 Hz, 1H); 4.65 (q, J=6.4 Hz, 1H); 4.56 (d, J=15.6 Hz, 1H); 2.48 (d, J=6.0 Hz, 3H); 1.48 (d, J=6.4 Hz, 3H).

E-189: LCMS (ES) n/z: 464.3 [M+H]⁺. ¹H NMR (400 MHz, DMSO-d₆): δ 9.41 (s, 1H); 8.51 (d, J=2.4 Hz, 1H); 7.89 (d, J=2.4 Hz, 1H); 7.87 (d, J=0.8 Hz, 1H); 7.40-7.35 (m, 1H); 7.24-7.20 (m, 1H); 7.16-7.10 (m, 2H); 7.10 (s, 1H); 4.92 (d, J=15.6 Hz, 1H); 4.65 (q, J=6.4 Hz, 1H); 4.56 (d, J=15.6 Hz, 1H); 2.48 (d, J=6.0 Hz, 3H); 1.48 (d, J=6.4 Hz, 3H).

### Example 190: Synthesis of 3-{[3-fluoro-2-(methylaminosulfonylamino)-4-pyridyl]methyl}-4-methyl-7-(1,3-oxazol-2-yloxy)-3,4-dihydro-2H-1-oxa-3,5-diazanaphthalen-2-one (E-190; Compound 190)

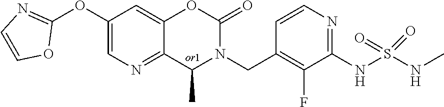

E-190 was synthesized by following similar synthesis sequence and experimental procedures as described for the synthesis of E-188, using key intermediates 102 and I-59 LCMS (ES) n/z: 465.3 [M+H]⁺. ¹H NMR (400 MHz, DMSO-d₆): δ 10.40 (s, 1H); 8.54 (d, J=2.4 Hz, 1H); 8.02 (d, J=4.8 Hz, 1H); 7.91 (d, J=2.4 Hz, 1H); 7.88 (s, 1H); 7.07 (s, 1H); 7.06-7.02 (m, 2H); 4.85 (d, J=16.4 Hz, 1H); 4.78 (q, J=6.4 Hz, 1H); 4.67 (d, J=16.4 Hz, 1H); 2.47 (d, J=6.0 Hz, 3H); 1.50 (d, J=6.8 Hz, 3H).

### Example 191: Synthesis of 3-{[3-fluoro-2-(methylaminosulfonylamino)-4-pyridyl]methyl}-4-methyl-7-(1,3-oxazol-2-yloxy)-3,4-dihydro-2H-1-oxa-3,5-diazanaphthalen-2-one (E-191; Compound 191)

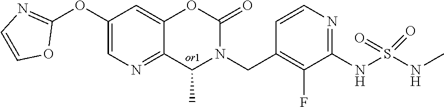

E-191 was synthesized by following similar synthesis sequence and experimental procedures as described for the synthesis of E-188, using key intermediates I-59 and 102. LCMS (ES) m/z: 465.3 [M+H]⁺. ¹H NMR (400 MHz, DMSO-d₆): δ 10.40 (s, 1H); 8.54 (d, J=2.4 Hz, I H); 8.02 (d, J=4.8 Hz, I H); 7.91 (d, J=2.4 Hz, I H); 7.88 (s, 1H); 7.07 (s, 1H); 7.05-7.01 (m, 2H); 4.85 (d, J=16.4 Hz, 1H); 4.78 (q, J=6.4 Hz, 1H); 4.66 (d, J=16.4 Hz, 1H); 2.47 (d, J=6.0 Hz, 3H); 1.50 (d, J=6.8 Hz, 3H).

Example 192: Synthesis of 3-{[3-fluoro-2-(methylaminosulfonylamino)-4 pyridyl]methyl}-4-methyl-7-(3-pyridazinyloxy)-3,4-dihydro-2H-1-oxa-3,5 diazanaphthalen-2-one (E-192; Compound 192)

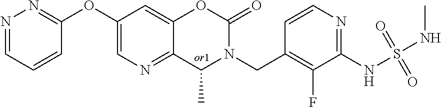

E-192 was synthesized by following similar synthesis sequence and experimental procedures as described for the synthesis of E-175, using key intermediates I-59 and 102. LCMS (ES) m/z: 476.3 [M+H]⁺. ¹H NMR (400 MHz, DMSO-d₆): δ 10.40 (s, 1H); 9.06 (d, J=4.4 Hz, 1H); 8.39 (d, J=2.0 Hz, 1H); 8.03 (s, 1H); 7.86-7.82 (m, 1H); 7.72 (d, J=2.0 Hz, 1H); 7.61 (d, J=8.8 Hz, 1H); 7.08-6.94 (m, 2H); 4.87 (d, J=16.0 Hz, 1H); 4.78 (q, J=6.4 Hz, 1H); 4.66 (d, J=16.0 Hz, 1H); 2.46 (d, J=3.4 Hz, 3H); 1.53 (d, J=6.4 Hz, 3H).

### Example 193: Synthesis of 3-{[3-fluoro-2-(methylaminosulfonylamino)-4 pyridyl]methyl}-4-methyl-7-(3-pyridazinyloxy)-3,4-dihydro-2H-1-oxa-3,5 diazanaphthalen-2-one (E-193; Compound 193)

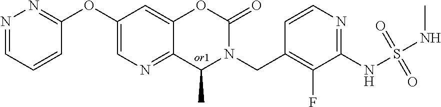

E-193 was synthesized by following similar synthesis sequence and experimental procedures as described for the synthesis of E-175, using key intermediates I-59 and 102. LCMS (ES) n/z: 476.3 [M+H]⁺. ¹H NMR (400 MHz, DMSO-d₆): b 10.40 (s, 1H); 9.07 (d, J=4.4 Hz, 1H); 8.39 (d, J=2.0 Hz, 1H); 8.03 (s, 1H); 7.86-7.82 (m, 1H); 7.72 (d, J=2.0 Hz, 1H); 7.61 (d, J=8.8 Hz, 1H); 7.06-6.96 (m, 2H); 4.87 (d, J=16.0 Hz, 1H); 4.78 (q, J=6.4 Hz, 1H); 4.66 (d, J=16.0 Hz, 1H); 2.46 (d, J=3.4 Hz, 3H); 1.54 (d, J=6.4 Hz, 3H).

### Example 194: Synthesis of 3-{[2,5-difluoro-3-(methylaminosulfonylamino)phenyl]methyl}-7-(3-pyridazinyloxy)-2H,3H-spiro[1,3-benzoxazine-4,1′-cyclobutan]-2-one (E-194; Compound 194)

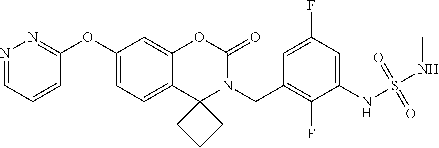 [SMILES: CNS(=O)(=O)Nc1cc(F)cc(CN2C(=O)Oc3cc(Oc4cccnn4)ccc3C23CCC3)c1F]

E-194 was synthesized by following similar synthesis sequence and experimental procedures as described for the synthesis of E-128, using key intermediates I-39 and I-61. LCMS (ESI) m/z: 518.4 [M+H]⁺. ¹H NMR (400 MHz, DMSO-d₆): δ 9.85 (s, 1H); 9.05 (d, J=4.4 Hz, 1H); 7.83-7.78 (m, 2H); 7.54-7.48 (m, 2H); 7.18-7.12 (m, 2H); 7.07 (d, J=2.0 Hz, 1H); 6.64-6.58 (m, 1H); 4.97 (s, 2H); 2.86-2.78 (m, 2H); 2.52 (d, J=3.4 Hz, 3H); 2.42-2.36 (m, 2H); 2.02-1.95 (m, 2H).

### Example 195: Synthesis of 3-{[2,5-difluoro-3-(methylaminosulfonylamino)phenyl]methyl}-7-(1,3-oxazol-2-yloxy)-2H,3H-spiro[1,3-benzoxazine-4,1′-cyclobutan]-2-one (E-195; Compound 195)

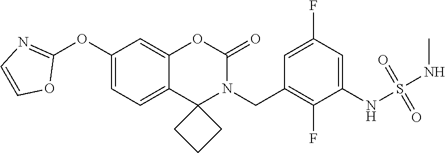 [SMILES: CNS(=O)(=O)Nc1cc(F)cc(CN2C(=O)Oc3cc(Oc4ncco4)ccc3C23CCC3)c1F]

E-195 was synthesized by following similar synthesis sequence and experimental procedures as described for the synthesis of E-130, using key intermediates I-39 and I-61. LCMS (ESI) m/z: 507.4 [M+H]1. ¹H NMR (400 MHz, DMSO-d₆): δ 9.84 (s, 1H); 7.86 (s, 1H); 7.80 (d, J=8.8 Hz, 1H); 7.48 (bs, 1H); 7.33-7.30 (m, 1H); 7.25 (d, J=2.4 Hz, 1H); 7.17-7.12 (m, 1H); 7.06 (s, 1H); 7.00 (d, J=4.8 Hz, 1H); 6.57 (bs, 1H); 4.95 (s, 2H); 2.83-2.75 (m, 2H); 2.37-2.32 (m, 2H); 2.07-1.93 (m, 2H).

### Example 196: Synthesis of 3-(3-amino-2-fluorobenzyl)-7-(pyridazin-3-yloxy)spiro[benzo[e][1,3]oxazine-4,1′-cyclopropan]-2(3H)-one (E-196; Compound 196)

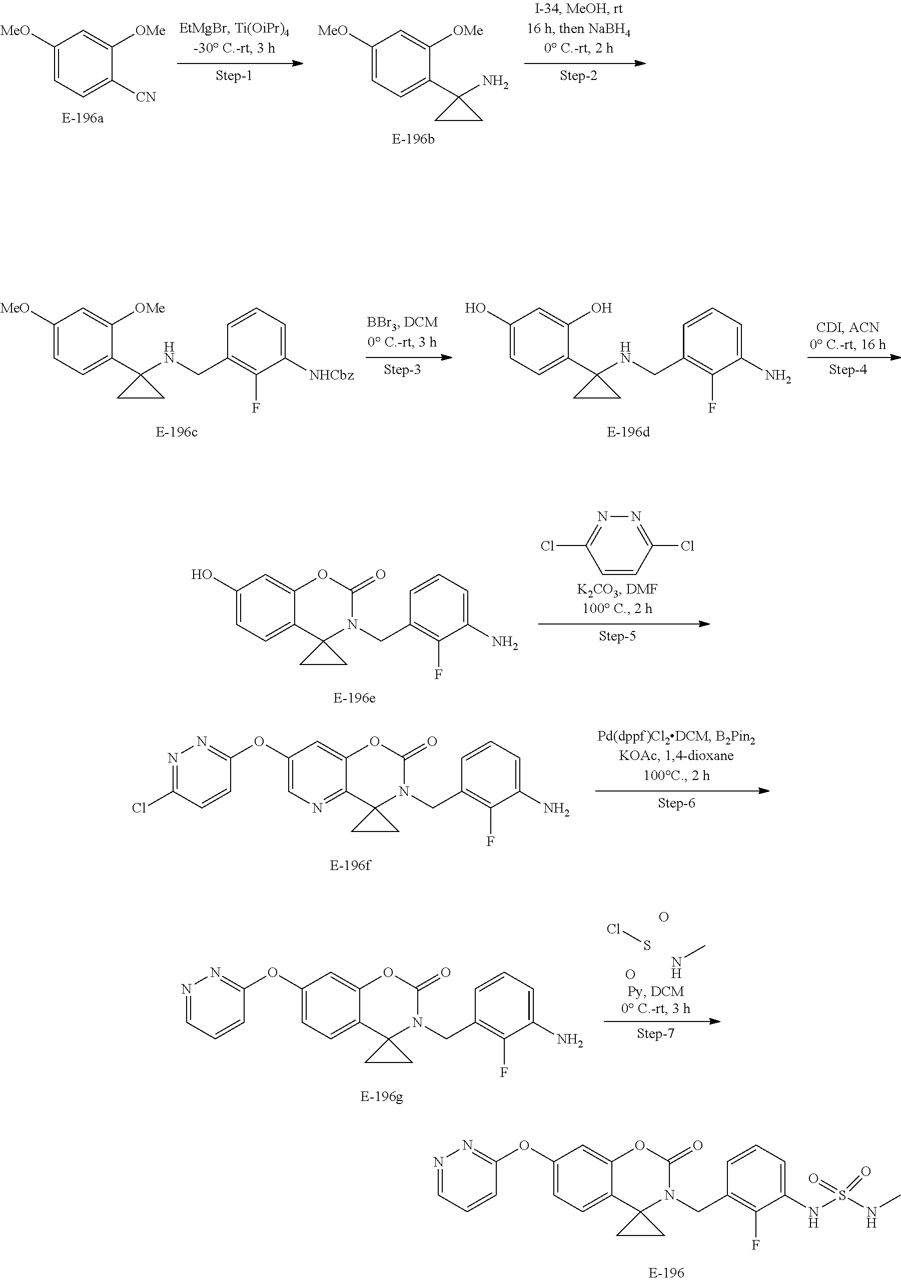

Step-1: Synthesis of 1-(2,4-dimethoxyphenyl)cyclopropan-1-amine (E-196b): To a stirred solution of 2,4-dimethoxybenzonitrile (E-196a) (6.0 g, 36.8 mmol) and titanium isopropoxide (7.94 g, 73.5 mmol) in anhydrous THE (90 mL) was added EtMgBr (10.8 g, 80.9 mmol) drop wise at −70° C. The reaction mixture was then allowed to stir at room temperature for 45 min. After 45 min, BF₃.Et₂O (10.4 g, 73.5 mmol) was added in one portion to the reaction mixture at room temperature and the reaction mixture was stirred for additional 3 h. The reaction mixture was then quenched by addition of a 2N HCl solution and stirred for 15 min. It was then neutralized by addition of a 3 N aqueous NaOH solution (50 mL). Extraction was carried out using EtOAc (5×30 mL); the combined organic extracts were washed with brine solution (50 mL), dried over anhydrous Na₂SO₄, filtered and evaporated under reduced pressure. The obtained crude compound (196b) was used in next reaction without further purification.

Step-2: Synthesis of benzyl (3-(((1-(2,4-dimethoxyphenyl)cyclopropyl)amino)methyl)-2-fluorophenyl)carbamate (E-196c): To a stirred solution of 1-(2,4-dimethoxyphenyl)cyclopropylamine (E-196b) (3.0 g, 15.5 mmol) in MeOH (60 mL) was added benzyl (2-fluoro-3-formylphenyl)carbamate (I-34) (2.93 g, 10.7 mmol) and stirred for 16 h at room temperature. The reaction mixture was then cooled to 0° C. and sodium borohydride (0.608 g, 16.1 mmol) was added to it. The reaction mixture was stirred at room temperature for additional 1 h, while monitoring reaction progress by TLC/LCMS. After completion of the reaction (1 h), ice water (30 mL) was added to it and extraction was carried out using EtOAc (3×30 mL). The combined organic extracts were washed with brine solution (30 mL), dried over anhydrous Na₂SO₄, filtered and evaporated under reduced pressure. The residue was purified by Combi-Flash (using gradient elution of 0-30% of EtOAc in heptane) to afford desired compound 196c (1.5 g) as a brown oil. LCMS (ES) m/z: 451.20 [M+H]⁺.

Step-3: Synthesis of 4-(1-((3-amino-2-fluorobenzyl)amino)cyclopropyl)benzene-1,3-diol (E-196d): To a stirred solution of benzyl (3-(((1-(2,4-dimethoxyphenyl)cyclopropyl)amino)methyl)-2-fluorophenyl)carbamate (E-196c) (1.5 g, 3.33 mmol) in anhydrous DCM (30 mL) was added BBr₃ (1.9 mL, 20 mmol) drop wise at −70° C. After complete addition, the reaction mixture was warmed to room temperature, while monitoring reaction progress by TLC/LCMS. After completion of the reaction (3 h), the reaction mixture was quenched by addition of ice cold water (20 mL). It was then neutralized by addition of saturated NaHCO₃ solution and extraction was carried out using DCM (3×30 mL). The combined organic extracts were washed with brine solution (30 mL), dried over anhydrous Na₂SO₄, filtered and evaporated the solvent under reduced pressure to afford the desired compound E-196d (0.8 g) as brown solid. The crude was used directly for the next step without further purification. LCMS (ES) m/z: 289.1 [M+H]⁺.

Step-4: Synthesis of 3-(3-amino-2-fluorobenzyl)-7-hydroxyspiro[benzo[e][1,3]oxazine-4,1′-cyclopropan]-2(3H)-one (E-196e): To a stirred solution of 4-(1-((3-amino-2-fluorobenzyl)amino)cyclopropyl)benzene-1,3-diol (E-196d) (0.8 g, 2.77 mmol) in acetonitrile (20 mL) was added CDT (0.675 g, 4.16 mmol) at 0° C. and the reaction mixture was stirred at room temperature for 16 h. The reaction progress was monitored by TLC/LCMS. After completion of the reaction, the reaction mixture was diluted with ice water (30 mL) and extracted with EtOAc (2×30 mL). The combined organic extracts were washed with brine solution (2×20 mL), dried over anhydrous Na₂SO₄, filtered and evaporated under reduced pressure. The residue was purified by Combi-Flash (using gradient elution of 0-70% of EtOAc in heptane) to afford desired compound E-196e (0.350 g) as a brown sticky solid. LCMS (ES) m/z: 315.20 [M+H]⁺.

Step-5: Synthesis of 3-(3-amino-2-fluorobenzyl)-7-((6-chloropyridazin-3-yl)oxy)spiro[benzo[e][1,3]oxazine-4,1′-cyclopropan]-2(3H)-one (E-196f): To a stirred solution of 3-[(3-amino-2-fluorophenyl)methyl]-7-hydroxy-2H,3H-spiro[1,3-benzoxazine-4,1′-cyclopropan]-2-one (E-196e) (0.35 g) in DMF (10 mL) was added potassium carbonate (0.308 g, 2.23 mmol) and 3,6-dichloropyridazine (0.332 g, 2.23 mmol). The reaction mixture was stirred at 100° C. for 3 h, while monitoring reaction progress by TLC/LCMS. After completion of the reaction (3 h), the reaction mixture was cooled to room temperature and ice water (30 mL) was added to it. Extraction was carried out using EtOAc (3×30 mL); the combined organic extracts were washed with brine solution (2×20 mL), dried over anhydrous Na₂SO₄, filtered and evaporated under reduced pressure to afford crude product E-196f (0.25 g) as a brown sticky solid, which was used in the next reaction without further purification. LCMS (ES) m/z: 427.20 [M+H]⁺.

Step-6: Synthesis of 3-(3-amino-2-fluorobenzyl)-7-(pyridazin-3-yloxy)spiro[benzo[e][1,3]oxazine-4,1′-cyclopropan]-2(3H)-one (E-196g): Argon gas was purged through a suspension of 3-(3-amino-2-fluorobenzyl)-7-((6-chloropyridazin-3-yl)oxy)spiro[benzo[e][1,3]oxazine-4,1′-cyclopropan]-2(3H)-one (E-196f) (0.25 g, 0.586 mmol), potassium acetate (0.172 g, 1.76 mmol) and bis(pinacolato)diboron (0.164 g, 644 μmol) in anhydrous 1,4-dioxane (15 mL) for 15 min before addition of PdCl₂(dppf). DCM (0.048 g, 0.059 mmol). The reaction mixture was stirred at 100° C. for 2 h, while monitoring reaction progress by TLC/LCMS. After complete consumption of starting material (2 h), the reaction mixture was cooled to room temperature and water (20 mL) was added to it. Extraction was carried out using EtOAc (3×30 mL), the combined organic extracts were washed with brine (30 mL), dried over anhydrous sodium sulphate, filtered and concentrated under reduced pressure. The residue was purified by Combi-flash (using gradient elution of 0-70% EtOAc in heptane) to obtain pure compound E-196g (0.140 g) as an off-white solid. LCMS (ESI) m/z: 393.20 [M+H]⁺.

Step-7: Synthesis of 3-{[2-fluoro-3-(methylaminosulfonylamino)phenyl]methyl}-7-(3-pyridazinyloxy)-2H,3H-spiro[1,3-benzoxazine-4,1′-cyclopropan]-2-one (E-196): To a stirred solution of 3-(3-amino-2-fluorobenzyl)-7-(pyridazin-3-yloxy)spiro[benzo[e][1,3]oxazine-4,1′-cyclopropan]-2(3H)-one (E-196g) (0.130 g, 0.331 mmol) in DCM (10 mL) at 0° C. was added pyridine (0.133 mL, 1.66 mmol) and N-methyl(chlorosulfonyl)amine (0.064 g, 0.497 mmol). The reaction mixture was stirred at room temperature for 3 h, while monitoring reaction progress by TLC and LCMS. After completion of the reaction, ice water (20 mL) was added to it and extraction was carried out using 5% MeOH in DCM (3×15 mL). The combined organic extract was washed with brine solution (20 mL), dried over anhydrous Na₂SO₄, filtered and evaporated under reduced pressure. The residue was purified by Combi-Flash (using gradient elution of 0-70% of EtOAc in heptane) followed by further purification by reverse phase preparative HPLC [using XBridge C18 (250×19 mm), 5 μm; 5 mM ammonium bicarbonate in water and acetonitrile as mobile phase with 18 mL/min flow rate] to afford E-196 (0.071 g) as an off-white solid. LCMS (ESI) m/z: 486.3 [M+H]⁺. ¹H NMR (400 MHz, DMSO-d₆): δ 9.04 (dd, J₁=0.8, J₂=4.0 Hz, 1H); 8.44 (bs, 1H); 7.81-7.77 (m, 1H); 7.51 (dd, Jᵢ=0.8, J₂=8.8 Hz, 1H); 7.33 (t, J=7.2 Hz, 1H); 7.22-7.18 (m, 3H); 7.01 (dd, J₁=2.0, J₂=8.4 Hz, 1H); 6.90 (t, J=6.8 Hz, 1H); 4.52 (s, 2H); 2.50 (d, J=3.4 Hz, 3H); 1.42 (t, J=6.4 Hz, 2H); 1.22 (t, J=7.2 Hz, 2H).

### Example 197: Synthesis of Synthesis of 3-{[2,6-difluoro-3-(methylaminosulfonylamino)phenyl]methyl}-7-(3-pyridazinyloxy)-2H,3H-spiro[1,3-benzoxazine-4,1′-cyclobutan]-2-one (E-197; Compound 197)

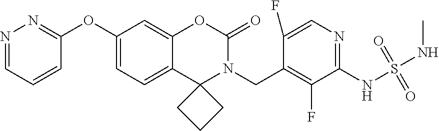 [SMILES: CNS(=O)(=O)Nc1ncc(F)c(CN2C(=O)Oc3cc(Oc4cccnn4)ccc3C23CCC3)c1F]

E-197 was synthesized by following similar synthesis sequence and experimental procedures as described for the synthesis of E-128, using key intermediates I-39 and I-62. LCMS (ES) m/z: 518.3 [M+H]⁺. ¹H NMR (400 MHz, DMSO-d₆): δ 9.28 (s, 1H); 9.03 (dd, J₁=0.8 Hz, J₂=4.4 Hz, 1H); 7.82-7.78 (m, 1H); 7.73 (d, J=8.8 Hz, 1H); 7.52 (d, J=8.8 Hz, 1H); 7.35-7.29 (m, 1H); 7.11 (dd, J₁=2.0 Hz, J₂=8.8 Hz, 1H); 7.05-6.91 (m, 3H); 5.00 (s, 2H); 3.02-2.90 (m, 2H); 2.44 (d, J=4.0 Hz, 3H); 2.38-2.30 (m, 2H); 2.00-1.90 (m, 2H).

### Example 198: Synthesis of 3-{[3-fluoro-2-(methylaminosulfonylamino)-4-pyridyl]methyl}-4-methyl-7-(1,2,4-triazin-3-yloxy)-3,4-dihydro-2H-1-oxa-3,5-diazanaphthalen-2-one (E-198; Compound 198)

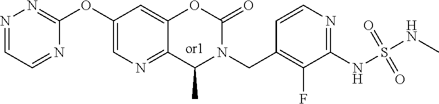 [SMILES: CNS(=O)(=O)Nc1nccc(CN2C(=O)Oc3cc(Oc4nccnn4)cnc3[C@@H]2C)c1F]

E-198 was synthesized by following similar synthesis sequence and experimental procedures as described for the synthesis of E-128, using key intermediates I-59 and 102. LCMS (ES) m/z: 477.3 [M+H]⁺. ¹H NMR (400 MHz, DMSO-d₆): δ 10.41 (s,1H); 9.35 (d, J=2.4 Hz, 1H); 8.81 (d, J=2.4 Hz, 1H); 8.47 (d, J=2.4 Hz, 1H); 8.04 (d, J=4.8 Hz, 1H); 7.82 (d, J=2.4 Hz, 1H); 7.10-7.00 (m, 2H); 4.86 (d, J=16.4 Hz, 1H); 4.79 (q, J=6.8 Hz, 1H); 4.67 (d, J=16.4 Hz, 1H); 2.47 (d, J=4.0 Hz, 3H); 1.53 (d, J=6.8 Hz, 3H).

### Example 199: Synthesis of 3-{[3-fluoro-2-(methylaminosulfonylamino)-4-pyridyl]methyl}-4-methyl-7-(1,2,4-triazin-3-yloxy)-3,4-dihydro-2H-1-oxa-3,5-diazanaphthalen-2-one (E-199; Compound 199)

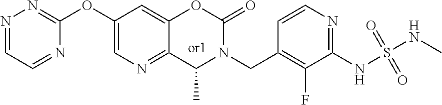 [SMILES: CNS(=O)(=O)Nc1nccc(CN2C(=O)Oc3cc(Oc4nccnn4)cnc3[C@H]2C)c1F]

E-199 was synthesized by following similar synthesis sequence and experimental procedures as described for the synthesis of E-128, using key intermediates I-59 and 102. LCMS (ES) m/z: 477.3 [M+H]⁺. ¹H NMR (400 MHz, DMSO-d₆): δ 10.41 (s, 1H); 9.34 (d, J=2.0 Hz, I H); 8.80 (d, J=2.4 Hz, I H); 8.47 (d, J=2.4 Hz, I H); 8.03 (d, J=4.8 Hz, I H); 7.82 (d, J=2.4 Hz, 1H); 7.08-7.00 (m, 2H); 4.86 (d, J=16.4 Hz, 1H); 4.81 (q, J=6.4 Hz, 1H); 4.67 (d, J=16.4 Hz, 1H); 2.47 (d, J=4.0 Hz, 3H); 1.53 (d, J=6.8 Hz, 3H).

### Example 200: Synthesis of 3-{[2,5-difluoro-3-(methylaminosulfonylamino)phenyl]methyl}-7-(3-pyridazinyloxy)-3,4-dihydro-2H-1,3-benzoxazin-2-one (E-200; Compound 200)

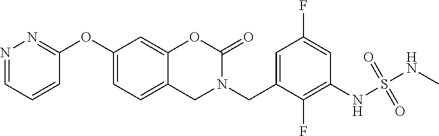 [SMILES: CNS(=O)(=O)Nc1cc(F)cc(CN2Cc3ccc(Oc4cccnn4)cc3OC2=O)c1F]

E-200 was synthesized by following similar synthesis sequence and experimental procedures as described for the synthesis of E-110, using key intermediates I-21 and I-61. LCMS (ES) m/z: 478.3 [M+H]⁺. ¹H NMR (400 MHz, DMSO-d₆): δ 9.84 (s, 1H); 9.02 (dd, J₁=0.8 Hz, J₂=4.8 Hz, 1H); 7.81-7.76 (m, 1H); 7.51-7.40 (m, 2H); 7.32 (d, J=8.4 Hz, 1H); 7.24-7.19 (m, 1H); 7.03 (s, 1H); 7.01-7.00 (m, 2H); 4.67 (s, 2H); 4.54 (s, 2H); 2.49 (d, J=4.0 Hz, 3H).

### Example 201: Synthesis of 2′-{[2-fluoro-3-(methylaminosulfonylamino)phenyl]methyl}-6′-(3-pyridazinyloxy)-2′H,3′H-4′-oxa-2′,8′-diazaspiro[cyclopropane-1,1′-naphthalen]-3′-one (E-201; Compound 201)

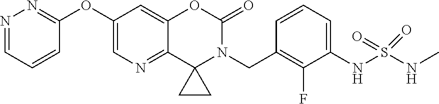 [SMILES: CNS(=O)(=O)Nc1cccc(CN2C(=O)Oc3cc(Oc4cccnn4)cnc3C23CC3)c1F]

E-201 was synthesized by following similar synthesis sequence and experimental procedures as described for the synthesis of E-110, using key intermediates I-32 and I-64. LCMS (ES) m/z: 487.4 [[M+H]⁺. ¹H NMR (400 MHz, DMSO-d₆): b 9.43 (s, 1H); 9.06 (dd, J₁=1.2 Hz; J₂=4.4 Hz, 1H); 8.32 (d, J=2.4 Hz; 1H); 7.85-7.81 (m, 1H); 7.71 (d, J=2.4 Hz, 1H); 7.61 (dd, J₁=1.2 Hz; J₂=9.2 Hz, 1H); 7.34 (t, J=7.6 Hz 1H); 7.24 (q, J=4.4 Hz, 1H); 7.12 (t, J=7.6 Hz, 1H); 7.02 (t, J=6.8 Hz, 1H); 4.51 (s, 2H); 2.52 (d, J=4.8 Hz, 3H); 1.50-1.46 (m, 2H); 1.40-1.36 (m, 2H).

### Example 202: Synthesis of 2′-{[3-fluoro-2-(methylaminosulfonylamino)-4-pyridyl]methyl}-6′-(3-pyridazinyloxy)-2′H,3′H-4′-oxa-2′,8′-diazaspiro[cyclopropane-1,1′-naphthalen]-3′-one (E-202; Compound 202)

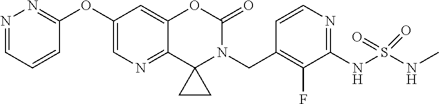 [SMILES: CNS(=O)(=O)Nc1nccc(CN2C(=O)Oc3cc(Oc4cccnn4)cnc3C23CC3)c1F]

E-202 was synthesized by following similar synthesis sequence and experimental procedures as described for the synthesis of E-110, using key intermediates I-35 and I-64. LCMS (ES) mn/z: 488.3 [M+H]⁺. ¹H NMR (400 MHz, DMSO-d₆) δ 10.40 (s, 1H); 9.07 (d, J=4.0 Hz, 1H); 8.33 (d, J=1.6 Hz, 1H); 8.02 (d, J=4.4 Hz, 1H); 7.85-7.82 (m, 1H); 7.71 (d, J=2 Hz, 1H); 7.61 (d, J=8.8 Hz, 1H); 7.04-6.90 (m, 2H); 4.52 (s, 2H); 2.50 (d, J=4.8 Hz, 3H); 1.52-1.48 (m, 2H); 1.42-1.36 (m, 2H).

### Example 203: Biological Assays

Protein expression and purification: BRAF (445-723) with N-term His-tag and C-term StrepII tag was expressed using the Expi293 system (ThermoFisher) and purified using Strep-Tactin® XT-Superflow (IBA) chromatography. ERK1 (full-length) with N-term His-tag was expressed in Rosetta DE3 cells (Millipore) and purified using HisTrap HP (Cytiva) and Superdex 200 (Cytiva) column chromatography. Full-length MEK1 for TR-FRET assays with N-term His-tag was expressed in BL21DE3 cells (ThermoFisher) and purified using HisTrap HP (Cytiva) and Superdex 200 (Cytiva) column chromatography.

Efficacy of exemplary compounds in inhibition of MEK1 kinase activity: The effect of compound MEK1 inhibition was evaluated using an ADP detection system for kinases, such as the ADP-Glo assay (Promega). The MEK1 kinase cascade assay consisted of 9 μL reaction volumes in 384-well white, opaque Optiplates (PerkinElmer). Compounds were prepared from DMSO stocks in assay buffer (10 mM HEPES pH 7.5, 5 mM MgCl₂, 2 mM CHAPS, and 1 mM DTT) and preincubated with 25 nM BRAF for 20 mins at 25° C. prior to addition of 6.25 nM MEK1/0.5 μM ERK/1 mM ATP premix to initiate the kinase reaction. Both BRAF and ERK1 were produced in-house, and MEK1 was purchased from SignalChem (M02-14H). DMSO was kept constant at a final concentration of 1%. After a 1 hr incubation at 25° C., conversion of ATP to ADP was detected using ADP-Glo™ reagents according to the manufacturer's protocol. Luminescence was measured using a CLARIOstar microplate reader (BMG). Percent (%) Inhibition was calculated by normalizing to DMSO control (max) and 1 μM Trametinib (min). % inhibition as a function of compound concentration from 2 replicate wells was fit with a 4-parameter logistic curve equation using GraphPad Prism or CDD Vault to derive IC₅₀ values.

Efficacy of exemplary compounds in stabilization of MEK1 and BRAF interaction: Compound stabilization of BRAF-MEK interaction was evaluated using TR-FRET (Time Resolved Fluorescence Energy Transfer). The FRET assay consisted of His-tagged MEK1 (produced in-house), GST-tagged BRAF (SignalChem, B08-11G), Anti His-Tb Cryptate donor, and Anti GST-XL665 acceptor fluorophores (PerkinElmer) to generate FRET signal upon BRAF-MEK1 binding. Compounds were prepared from DMSO stocks in assay buffer (50 mM Tris pH 7.5, 150 mM NaCl, 1 mM DTT, 0.05% Tween-20, 5 mM MgCl₂, and 100 μM ATP) and incubated with 4 nM BRAF and 10 nM MEK for 1 hr at 25° C. 1 ng Anti His-Tb Cryptate/20 ng Anti GST-XL665 premix was then added and incubated for 1 hr at 25° C. TR-FRET was measured using Cytation5 microplate reader (Agilent) using Ex/Em=340/620 nm and Ex/Em=340/665 nm to determine the FRET ratio. Fold stabilization was calculated as FRET signal of treated well/DMSO control. Fold stabilization as a function of compound concentration from 2 replicate wells was fit with a 4-parameter logistic curve equation using GraphPad Prism or CDD Vault to derive EC₅₀ values.

Cell culture: HCTI 16, HT-29, and NCI-H292 cells were acquired from American Type Culture Collection (ATCC). HCT116 and HT-29 cells were maintained in ATCC-formulated McCoy's 5a Medium Modified supplemented with 10% fetal bovine serum, NCI-H292 cells were cultured in ATCC formulated RPMI-1640 supplemented with 10% fetal bovine serum. All cells were maintained at 37° C., 5% CO₂.

Efficacy of exemplary compounds in cellular inhibition of ERK phosphorylation: Phosphorylation status of ERK1/2 (p-ERK) was used as a readout for inhibition of BRAF-MEK pathway. p-ERK was detected using an immunoassay for quantitative detection of phospho-ERK 1/2 (phosphorylated on Thr202/Tyr204 in ERK1, or Thr185/Tyr187 in ERK2), such as the AlphaLISA® SureFire® Ultra™ p-ERK1/2 (Thr202/Tyr204) kit (Perkin Elmer). HCT116 cells were plated at 10,000 cells per well in 384-well TC-treated plates (Coming). Cells were plated for 24 hours, the medium was then aspirated and cells were treated with vehicle (DMSO) or test compounds (0.1% DMSO in final volume) prepared in serum free, phenol red free medium for 2 hours. Following treatment, cells were stimulated with PMA (1.25 μM/well) for 30 mins after which the medium was aspirated, and the cells were lysed for 10 mins with 1× lysis buffer supplemented with Halt Protease and Phosphatase Inhibitor Cocktail (Thermo Fisher). The lysate was transferred to a 384 well Alpha-plate (PerkinElmer) and incubated for 1 hour with acceptor bead mix followed by 1 hour incubation with donor bead mix. After incubation, the plate was read on a Cytation5 microplate reader (Agilent). % inhibition as a function of compound concentration from 3 replicate wells was fit with a 4-parameter logistic curve using GraphPad Prism or CDD Vault to derive IC₅₀ values.

Efficacy of exemplary compounds in inhibition of cell viability: HCT116 and HT-29 cells were plated at 4,000 cells per well and NCI-H292 cells were plated at 2,000 cells per well in their respective media in 384-well white, TC-treated plates (Corning). After 24 hours of incubation, the cells were treated with vehicle (DMSO) or test compounds (0.1% DMSO in final volume) for 72 hrs. The plate was equilibrated at room temperature for 10 mins followed by addition of a 1:1 volume of CellTiter-Glo® reagent (Promega). The contents were mixed for 2 mins on an orbital shaker to induce lysis. The plate was incubated at room temperature for 10 mins to stabilize luminescent signal before measuring luminescence on a Cytation5 microplate reader (Agilent). % inhibition as a function of compound concentration from 3 replicate wells was fit with a 4-parameter logistic curve using GraphPad Prism or CDD Vault to derive IC₅₀ values.

Results from this assay are summarized in Table 3 below. In this table, for the BRAF ADP Glo kinase assay, “A” indicates IC₅₀ of less than 5 nM; “B” indicates IC₅₀ of between 5 nM to 50 nM; and “C” indicates IC₅₀ of between 50 nM to 500 nM. “D” indicates IC₅₀ of greater than 500 nM. For the BRAF-MEK TR-FRET assay, “A” indicates EC₅₀ of less than 50 nM; “B” indicates EC₅₀ of between 50 nM to 200 nM; and “C” indicates EC₅₀ of between 200 nM to 1 μM. “D′” indicates EC₅₀ of greater than 1 μM. For the AlphaLisa P-ERK assay, “A” indicates IC₅₀ of less than 1 nM; “B” indicates IC₅₀ of between 1 nM to 10 nM; and “C” indicates IC₅₀ of between 10 nM to 100 nM. “D” indicates IC₅₀ of greater than 100 nM. For the cell viability assay, “A” indicates IC₅₀ of less than 1 nM; “B” indicates IC₅₀ of between 1 nM to 10 nM; and “C” indicates IC₅₀ of between 10 nM to 100 nM. “D” indicates IC₅₀ of greater than 100 nM.

TABLE 3
Assay Results

| | | | | Cell Viability Assay: |
|---|---|---|---|---|
| Compound | BRAF ADP Glo | BRAF-MEK TR- | AlphaLisa P-ERK | IC50 Determination |
| No. | kinase assay V3 | FRET Assay | Assay | NCI-H292 |
| 17 | A | A | A | N/A |
| 18 | B | A | C | N/A |
| 19 | B | A | A | N/A |
| 20 | A | A | B | N/A |
| 21 | B | C | B | N/A |
| 22 | B | B | C | N/A |
| 23 | B | A | N/A | N/A |
| 24 | B | B | N/A | N/A |
| 25 | A | A | N/A | N/A |
| 26 | C | D | N/A | N/A |
| 27 | D | D | N/A | N/A |
| 28 | B | C | N/A | N/A |
| 29 | C | D | N/A | N/A |
| 30 | D | D | N/A | N/A |
| 31 | B | A | N/A | N/A |
| 32 | B | C | N/A | N/A |
| 33 | B | B | N/A | N/A |
| 34 | A | C | N/A | N/A |
| 35 | A | B | C | C |
| 36 | B | B | N/A | N/A |
| 37 | B | B | N/A | N/A |
| 38 | A | A | N/A | A |
| 39 | B | A | N/A | B |
| 40 | B | B | C | B |
| 41 | A | A | B | B |
| 42 | A | B | B | B |
| 43 | A | A | N/A | B |
| 44 | A | C | N/A | N/A |
| 45 | D | D | N/A | N/A |
| 46 | C | D | N/A | N/A |
| 47 | C | D | N/A | N/A |
| 48 | B | C | N/A | N/A |
| 49 | B | C | N/A | N/A |
| 50 | B | D | N/A | N/A |
| 51 | B | C | N/A | C |
| 52 | B | B | B | B |
| 53 | B | B | C | B |
| 54 | C | D | N/A | N/A |
| 55 | B | C | N/A | N/A |
| 56 | B | C | N/A | C |
| 57 | B | B | B | B |
| 58 | A | A | A | A |
| 59 | B | D | B | B |
| 60 | C | D | N/A | N/A |
| 61 | B | D | N/A | N/A |
| 62 | B | D | D | D |
| 63 | B | B | B | B |
| 64 | A | B | B | B |
| 65 | A | D | C | C |
| 66 | A | C | B | B |
| 67 | B | C | B | B |
| 68 | B | B | C | B |
| 69 | C | D | N/A | N/A |
| 70 | C | D | N/A | N/A |
| 71 | B | C | C | B |
| 72 | B | B | B | B |
| 73 | B | D | N/A | B |
| 74 | B | D | N/A | B |
| 77 | B | B | N/A | A |
| 78 | B | C | N/A | C |
| 79 | B | D | N/A | C |
| 80 | B | C | C | B |
| 81 | A | B | B | B |
| 82 | A | C | B | B |
| 83 | B | C | N/A | N/A |
| 84 | B | D | N/A | N/A |
| 85 | B | C | N/A | N/A |
| 86 | B | C | N/A | N/A |
| 87 | C | D | N/A | N/A |
| 88 | D | D | N/A | N/A |
| 89 | A | C | N/A | N/A |
| 90 | B | D | N/A | N/A |
| 91 | A | C | N/A | N/A |
| 92 | B | D | N/A | N/A |
| 93 | B | D | N/A | N/A |
| 95 | A | B | B | B |
| 96 | A | B | N/A | N/A |
| 98 | D | D | N/A | N/A |
| 100 | B | C | N/A | N/A |
| 102 | B | B | N/A | B |
| 103 | B | C | N/A | B |
| 104 | B | C | N/A | N/A |
| 105 | A | A | B | B |
| 107 | B | C | N/A | N/A |
| 108 | B | B | N/A | A |
| 109 | B | C | N/A | N/A |
| 110 | B | C | N/A | N/A |
| 111 | B | D | N/A | N/A |
| 112 | A | C | N/A | B |
| 113 | B | C | N/A | B |
| 115 | A | B | B | A |
| 117 | B | C | B | C |
| 118 | A | B | B | A |
| 120 | A | B | N/A | N/A |
| 123 | B | C | N/A | C |
| 125 | B | C | N/A | C |
| 129 | N/A | C | N/A | N/A |
| 130 | N/A | B | N/A | N/A |
| 131 | A | B | B | B |
| 132 | B | C | N/A | N/A |
| 133 | B | D | C | C |
| 144 | N/A | D | N/A | N/A |
| 145 | N/A | D | N/A | N/A |
| 146 | N/A | D | N/A | N/A |
| 147 | N/A | B | N/A | N/A |
| 148 | B | D | C | C |
| 149 | A | B | B | B |
| 151 | N/A | D | N/A | N/A |
| 152 | B | C | N/A | N/A |
| 153 | B | C | N/A | N/A |
| 156 | B | C | N/A | N/A |
| 157 | B | D | N/A | N/A |
| 158 | N/A | D | N/A | N/A |
| 159 | N/A | D | N/A | N/A |
| 160 | N/A | C | N/A | N/A |
| 161 | N/A | B | C | B |
| 162 | N/A | B | C | B |
| 163 | N/A | C | N/A | N/A |
| 164 | B | A | C | A |
| 165 | A | A | N/A | B |
| 166 | B | A | N/A | B |
| 167 | B | B | N/A | C |
| 168 | B | C | N/A | N/A |
| 169 | B | A | N/A | B |
| 170 | B | C | N/A | C |
| 171 | A | A | N/A | N/A |
| 172 | C | D | N/A | N/A |
| 173 | B | A | N/A | N/A |
| 174 | B | A | N/A | N/A |
| 175 | B | B | N/A | N/A |
| 177 | A | A | N/A | N/A |
| 178 | B | C | N/A | N/A |
| 179 | B | C | N/A | N/A |
| 180 | B | D | N/A | N/A |
| 181 | B | C | N/A | N/A |
| 182 | B | D | N/A | N/A |
| 183 | C | C | N/A | N/A |
| 184 | C | B | N/A | N/A |
| 185 | B | B | N/A | N/A |
| 186 | C | C | N/A | N/A |
| 187 | B | B | N/A | N/A |
| 189 | C | D | N/A | N/A |
| 190 | B | D | N/A | N/A |
| 191 | C | B | N/A | N/A |
| 192 | B | C | N/A | N/A |
| 193 | B | A | N/A | N/A |
| 194 | B | B | N/A | N/A |
| 195 | C | D | N/A | N/A |
| 196 | A | A | N/A | N/A |
| 197 | C | D | N/A | N/A |
| 198 | B | A | N/A | N/A |
| 199 | B | B | N/A | N/A |
| 200 | B | C | N/A | N/A |
| 201 | A | A | N/A | N/A |
| 202 | A | A | N/A | N/A |

### INCORPORATION BY REFERENCE

The entire disclosure of each of the patent and scientific documents referred to herein is incorporated by reference for all purposes. This application refers to various issued patents, published patent applications, journal articles, and other publications, all of which are incorporated herein by reference.

If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

1-63. (canceled)

64. A compound of Formula (I): 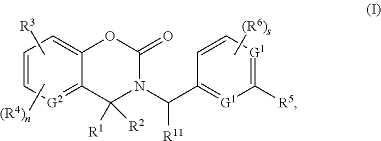
or a pharmaceutically acceptable salt thereof, wherein:
each G¹ is independently N or CH, wherein the H of CH may be substituted with R⁶; G² is N or CH, wherein the H of CH may be substituted with R³ or R⁴;
R¹ and R² are each independently selected from the group consisting of hydrogen, deuterium, halogen, and C₁₋₆ alkyl, wherein said C₁₋₆ alkyl is optionally substituted, or R¹ and R² may be taken together with the carbon atom to which they are attached to form C₃₋₆ cycloalkyl or 3-6 membered heterocyclyl ring, wherein the C₃₋₆ cycloalkyl or 3-6 membered heterocyclyl ring may be optionally substituted;
R³ is selected from the group consisting of —OR^{a}, —NR^{b}R^{c}, —C(O)N(R^{e})₂, C₁₋₆ alkyl optionally substituted with one or more R⁷, 3-10 membered heterocyclyl, 5-10 membered heteroaryl, C₃₋₁₀ cycloalkyl, and phenyl, wherein said 3-10 membered heterocyclyl, 5-10 membered heteroaryl, C₃₋₁₀ cycloalkyl, and phenyl are optionally substituted;
each R⁴ is independently selected from the group consisting of hydrogen, halogen, C₁₋₆ alkyl, C₁₋₆ haloalkyl, C₁₋₆ haloalkoxy, —CN, —N(R^{e})₂, NO₂, and C₁₋₆ alkoxy;
R^{a}, R^{b}, R^{c} and R^{d} are each independently selected from the group consisting of hydrogen, C₁₋₆ alkyl optionally substituted with one or more R⁷, —C(O)-(3-10 membered heterocyclyl or 5-10 membered heteroaryl), —C(O)N(R^{e})₂, —C(O)—(C₁₋₆ alkyl), —C(O)—(C₃₋₁₀ cycloalkyl), 5-10 membered heteroaryl, phenyl, 3-10 membered heterocyclyl, and C₃₋₁₀ cycloalkyl, wherein said 5-10 membered heteroaryl, phenyl, 3-10 membered heterocyclyl, and C₃₋₁₀ cycloalkyl are optionally substituted;
R⁵ is selected from the group consisting of —(C₁₋₆ alkylene)-S(O)ₚR^{h}, —NR^{e}S(O)ₚR^{h}, —NRC(O)R^{h}, —N(R^{e})₂, NO₂, —OR^{k}, —S(O)_{q}R^{h}, 3-10 membered heterocyclyl, 5-10 membered
heteroaryl, (C₁₋₆ alkylene)-(3-10 membered heterocyclyl), and (C₁₋₆ alkylene)-5-10 membered heteroaryl, wherein said 3-10 membered heterocyclyl and 5-10 membered heteroaryl are optionally substituted;
each R⁶ is independently selected from the group consisting of C₁₋₆ alkyl, halogen, C₁₋₆ haloalkyl, C₁₋₆ haloalkoxy, —CN, —N(R^{e})₂, NO₂, and C₁₋₆ alkoxy;
each R⁷ is selected from the group consisting of halogen, —C(O)N(R^{e})₂, —C(O)OR^{p}, 3-10 membered heterocyclyl, and C₃₋₁₀ cycloalkyl, wherein said 3-10 membered heterocyclyl and C₃₋₁₀ cycloalkyl are optionally substituted;
each R^{e} is independently hydrogen or C₁₋₆ alkyl;
each R^{h} is independently selected from the group consisting of —NRⁿR^{o}, C₁₋₆ alkyl optionally substituted with one or more R⁸, C₁₋₆ alkoxy, C₃₋₁₀ cycloalkyl, 3-10 membered heterocyclyl, phenyl, and 5-6 membered heteroaryl, wherein said C₃₋₁₀ cycloalkyl, 3-10 membered heterocyclyl, phenyl, and 5-6 membered heteroaryl are optionally substituted;
R^{k} is selected from the group consisting of C₁₋₆ alkyl optionally substituted with one or more R⁸, C₃₋₁₀ cycloalkyl, 3-10 membered heterocyclyl, phenyl, and 5-6 membered heteroaryl, wherein the C₃₋₁₀ cycloalkyl, 3-10 membered heterocyclyl, phenyl, and 5-6 membered heteroaryl are optionally substituted;
R⁸ is selected from the group consisting of —N(R^{e})₂, halogen, and C₁₋₆ alkoxy;
R¹¹ is hydrogen or C₁₋₆ alkyl;
Rⁿ and R^{o} are each independently selected from hydrogen, and C₁₋₆ alkyl optionally substituted with one or more halogen or C₁₋₆ alkoxy;
each R^{p} is independently hydrogen or C₁₋₆ alkyl;
n is selected from 0, 1, 2, and 3;
s is selected from 0, 1, 2, and 3; and
m, p, and q are each independently selected from 0, 1, and 2; wherein:
when R¹ and R² are each independently hydrogen or optionally substituted C₁₋₆ alkyl, and R⁵ is —NR^{e}S(O)ₚR^{h}, then R^{a} is selected from the group consisting of hydrogen, C₁₋₆ alkyl optionally substituted with one or more R⁷, —C(O)-(3-10 membered heterocyclyl or 5-10 membered heteroaryl), —C(O)—(C₁₋₆ alkyl), —C(O)—(C₃₋₁₀ cycloalkyl), 5-6 membered heteroaryl selected from the group consisting of pyridazinyl, oxazolyl, triazinyl, and pyrazinyl, phenyl, 3-10 membered heterocyclyl, and C₃₋₁₀ cycloalkyl, wherein said 5-6 membered heteroaryl, phenyl, 3-10 membered heterocyclyl, and C₃₋₁₀ cycloalkyl are optionally substituted.

65. The compound of claim 64, wherein said compound or a pharmaceutically acceptable salt thereof is selected from the group consisting of: 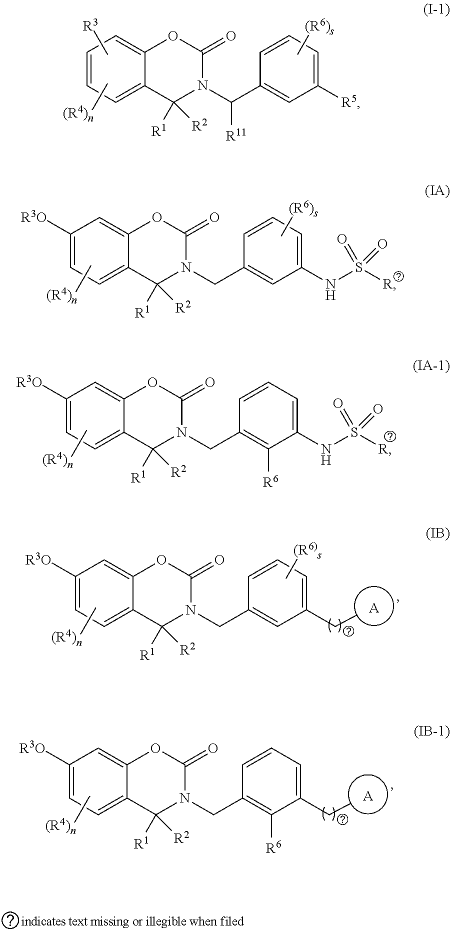
wherein in (IB) or (IB-1):
t is 0 or 1; 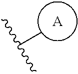
is a 3-10 membered heterocyclyl or a 5-10 membered heteroaryl, wherein said 3-10 membered heterocyclyl and said 5-10 membered heteroaryl are optionally substituted, 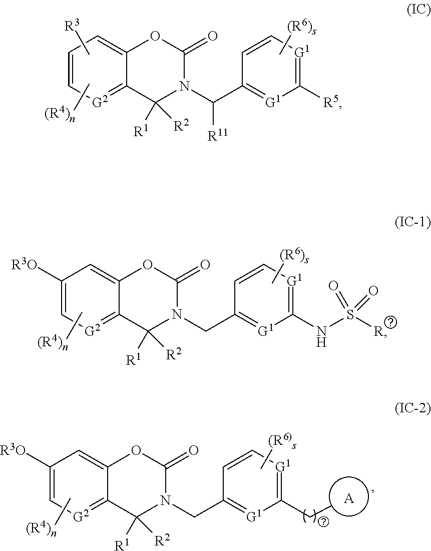
wherein in (IC), (IC-1) and (IC-2):
R¹ and R² are taken together with the carbon atom to which they are attached to form C₃₋₆ cycloalkyl or 3-6 membered heterocyclyl ring, wherein said C₃₋₆ cycloalkyl or 3-6 membered heterocyclyl ring may be optionally substituted; 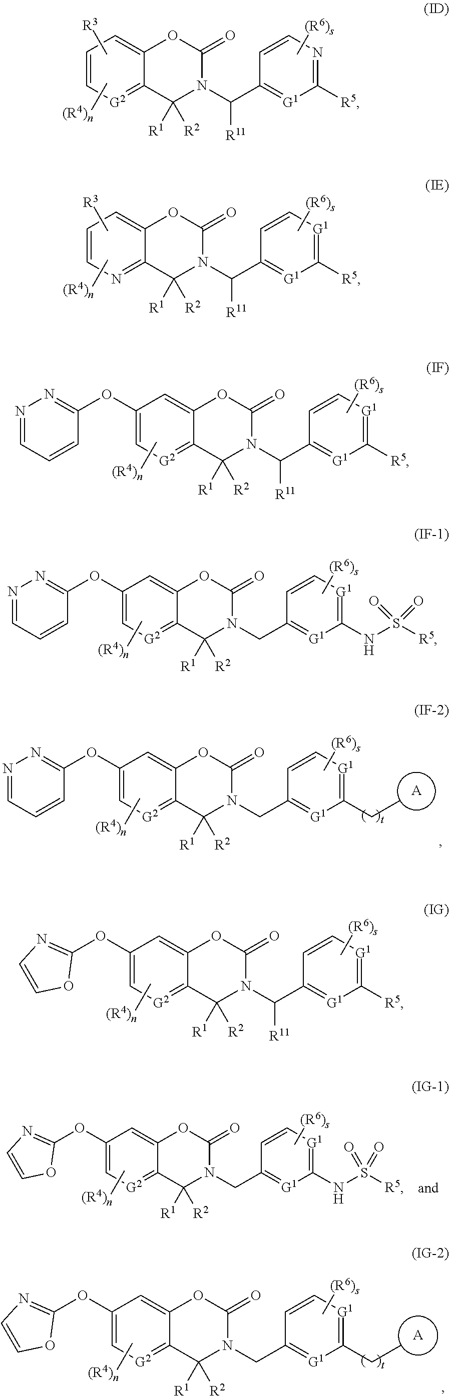
wherein said variables are as defined in claim 1.

66. The compound of claim 64, wherein R¹ and R² are taken together with a carbon atom to which they are attached to form cyclopropyl ring, cyclobutyl ring, oxetanyl ring, or azetidinyl ring, wherein said cyclopropyl ring, cyclobutyl ring, oxetanyl ring, or azetidinyl ring is optionally substituted with C₁₋₆ alkyl.

67. The compound of claim 64, wherein R³ is —OR^{a} or C₁₋₆ alkylene optionally substituted with one or more R⁷.

68. The compound of claim 67, wherein R³ is —OR^{a}.

69. The compound of claim 68, wherein R^{a} is selected from the group consisting of C(O)N(R^{e})₂, 5-10 membered heteroaryl, C₁₋₆ alkyl optionally substituted with one or more R⁷, C₃₋₁₀ cycloalkyl, —C(O)-(3-10 membered heterocyclyl), —C(O)—(C₁₋₆ alkyl), and —C(O)—(C₃₋₁₀ cycloalkyl).

70. The compound of claim 69, wherein R^{a} is 5-6 membered heteroaryl.

71. The compound of claim 70, wherein R^{a} is selected from the group consisting of.

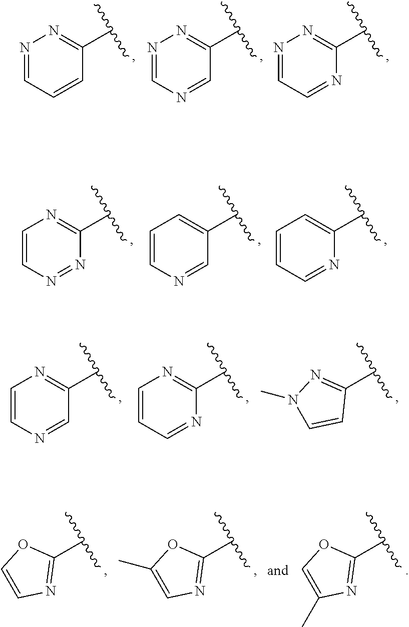

72. The compound of claim 4, wherein R^{a} is selected from the group f

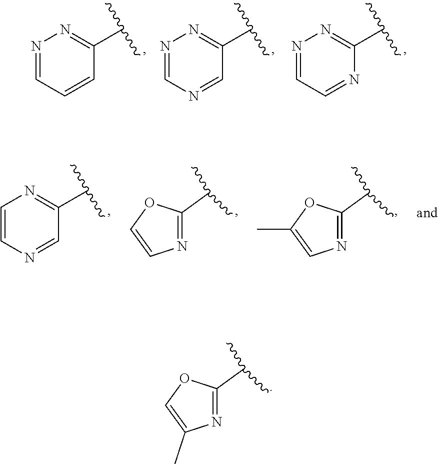

73. The compound of claim 64, wherein each R⁷ is independently selected from the group consisting of halogen, —C(O)N(R^{e})₂, 3-10 membered heterocyclyl, and C₃₋₁₀ cycloalkyl.

74. The compound of claim 73, wherein each R^{e} is hydrogen or C₁₋₆ alkyl.

75. The compound of claim 64, wherein each R⁴ is selected from the group consisting of halogen, C₁₋₆ alkyl, and C₁₋₆ haloalkyl.

76. The compound of claim 64, wherein R⁵ is selected from the group consisting of NR^{e}S(O)ₚR^{h}, NR^{e}C(O)R^{h}, and —(C₁₋₆ alkylene)-S(O)ₚR^{h}.

77. The compound of claim 64, wherein R^{h} is selected from the group consisting of —NRⁿR^{o}, C₁₋₆ alkoxy, C₃₋₁₀ cycloalkyl, and 3-10 membered heterocyclyl, wherein the C₃₋₁₀ cycloalkyl, 3-10 membered heterocyclyl, and 5-6 membered heteroaryl are optionally substituted with one or more substituents independently selected from the group consisting of halogen, C₁₋₆ alkyl, C₁₋₆ haloalkyl, C₁₋₆ haloalkoxy, C₁₋₆ alkoxy, and C(O)NR^{p}R^{q}, wherein R^{p} and R^{q} are independently hydrogen or C₁₋₆ alkyl.

78. The compound of claim 64 wherein each Rⁿ and R^{o} are independently selected from hydrogen, methyl, and —C₁₋₆ alkylene-C₁₋₆ alkoxy.

79. The compound of claim 64, wherein R^{h} is a 3-10 membered heterocyclyl optionally substituted with one or more substituents independently selected from the group consisting of a halogen, C₁₋₆ alkyl, C₁₋₆ haloalkyl, C₁₋₆ haloalkoxy, C₁₋₆ alkoxy, and C(O)NR^{p}R^{q}.

80. The compound of claim 64, wherein R⁵ is selected from the group consisting of a 3-10 membered heterocyclyl, a 5-10 membered heteroaryl, (C₁₋₆ alkylene)-3-10 membered heterocyclyl, and (C₁₋₆ alkylene)-5-10 membered heteroaryl wherein said 3-10 membered heterocyclyl, and said 5-10 membered heteroaryl are optionally substituted.

81. The compound of claim 65, wherein 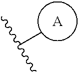
is selected from the group consisting of 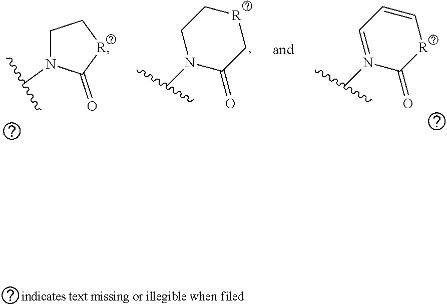
and wherein R⁹ is selected from the group consisting of O, NH, N—C₁₋₆ alkyl, N—C₁₋₆ alkoxy, and N—(C₁₋₆ alkylene)-C(O)N(R^{e}).

82. The compound of claim 64, wherein said compound is selected from Table 2.

83. A pharmaceutical composition comprising a compound of claim 64 or a pharmaceutically acceptable salt thereof and a pharmaceutically acceptable excipient.
